(19) United States
(12) Patent Application Publication
(10) Pub. No.: US 2026/0327580 A1
(43) Pub. Date: Oct. 22, 2026

(54) ENGINEERED DNA POLYMERASE VARIANTS

(71) Applicant: Codexis, Inc., Redwood City, CA (US)

(72) Inventors: Oscar Alvizo, Fremont, CA (US); Ericka Bermudez, Aptos, CA (US); Le Cai, Santa Clara, CA (US); Jonathan Dewayne Dorigatti, Newark, CA (US); David Elgart, San Mateo, CA (US); Jason Fell, Martinez, CA (US); Sandy M. Gomes, Redwood City, CA (US); Supriya Vijaykumar Kadam, San Jose, CA (US); Ambuj Kumar, Redwood City, CA (US); Michelle Li, Millbrae, CA (US); Marissa Greene MacAvoy, San Francisco, CA (US); Mathew G. Miller, San Carlos, CA (US); Vesna Mitchell, Santa Clara, CA (US); Jovana Nazor, Milpitas, CA (US); Nazanin Nahrjou, Mountain View, CA (US)

(21) Appl. No.: 19/112,171

(22) PCT Filed: Sep. 12, 2023
(86) PCT No.: PCT/US2023/073948

§ 371 Date: Mar. 14, 2025

Related U.S. Application Data
(60) Provisional application No. 63/375,661, filed on Sep. 14, 2022.
(60) Provisional application No. 63/500,119, filed on May 4, 2023.

(51) Int. Cl. C12N 9/12; C12Q 1/6806; C12Q 1/686; C12Q 1/6869

(52) U.S. Cl. CPC C12N 9/1252; C12Q 1/6806; C12Q 1/686; C12Q 1/6869; C07K 2319/00; C12N 2800/22

(57) ABSTRACT

The present invention relates to engineered DNA polymerase polypeptides and compositions thereof, as well as polynucleotides encoding the engineered DNA polymerase polypeptides. The present invention also provides methods of using the engineered DNA polymerase polypeptides or compositions thereof for diagnostic and other purposes.

### CROSS-REFERENCE TO RELATED APPLICATIONS

This application claims the benefit of U.S. Provisional Patent Application Ser. No. 63/375,661, filed Sep. 14, 2022, and U.S. Provisional Patent Application Ser. No. 63/500,119, filed May 4, 2023, both of which are incorporated by reference herein in their entireties.

### REFERENCE TO SEQUENCE LISTING, TABLE OR COMPUTER PROGRAM

The Sequence Listing concurrently submitted herewith as file name CX9-236WO1_ST26.xml with a file size of 19,716,660 bytes, created Sep. 11, 2023, is part of the specification and is incorporated by reference herein.

### FIELD OF THE INVENTION

The present invention provides engineered DNA polymerase polypeptides and compositions thereof, as well as polynucleotides encoding the engineered DNA polymerase polypeptides. The disclosure also provides methods for use of the recombinant DNA polymerase or compositions thereof for diagnostic, molecular biological tools, and other purposes.

### BACKGROUND

DNA polymerases are enzymes that synthesize DNA from deoxyribonucleotides. These enzymes are essential for DNA replication. There are various types of DNA polymerases displaying different properties and found in different types of organisms. Polymerases obtained from thermophilic organisms have found wide ranging vital uses in various in vitro methods, including but not limited to the polymerase chain reaction (PCR), nucleic sequencing, and other diagnostic, molecular biological, and forensic applications. While there are numerous commercially available thermostable DNA polymerases, such as Taq and Pfu DNA polymerases, a need remains in the art for thermostable enzymes with improved properties, such as enhanced processivity and/or fidelity.

### SUMMARY OF THE INVENTION

The present invention provides engineered DNA polymerase polypeptides and compositions thereof, as well as polynucleotides encoding the engineered DNA polymerase polypeptides. The present invention also provides methods of using the engineered DNA polymerase polypeptides and compositions thereof for diagnostic and other purposes.

The present invention provides engineered DNA polymerases, or a functional fragment thereof, comprising polypeptide sequences having at least 75%, 80%, 85%, 90%, 91%, 92%, 93%, 94%, 95%, 96%, 97%, 98%, 99%, or more sequence identity to a reference sequence comprising residues 12 to 841 of SEQ ID NO: 2, wherein the polypeptide sequence comprises one or more substitutions relative to the reference sequence comprising residues 12 to 841 of SEQ ID NO: 2.

In some embodiments, the present invention provides engineered DNA polymerases, or a functional fragment thereof, comprising polypeptide sequences having at least about 75%, about 80%, about 85%, about 90%, about 91%, about 92%, about 93%, about 94%, about 95%, about 96%, about 97%, about 98%, about 99%, or more sequence identity to a reference sequence comprising SEQ ID NO: 2, wherein the polypeptide sequence comprises one or more substitutions relative to the reference sequence of SEQ ID NO: 2.

In some additional embodiments, the present invention provides engineered DNA polymerases, or a functional fragment thereof, comprising a polypeptide sequence having at least about 75% or more sequence identity to a reference sequence comprising residues 12 to 841 of SEQ ID NO: 2, or to a reference sequence of SEQ ID NO: 2, wherein the polypeptide sequence comprises one or more substitutions relative to the reference sequence comprising residues 12 to 841 of SEQ ID NO: 2, or to the reference sequence of SEQ ID NO: 2.

In some additional embodiments, the present invention provides an engineered DNA polymerase, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2 or to the reference sequence of SEQ ID NO: 2.

In some embodiments, the engineered DNA polymerase comprises at least one substitutions or substitution sets at amino acid positions selected from 11/287, 15/633, 18/145/255, 20/602, 29, 50/255/557/685, 110, 113/170/194/235/249/319/645/659/660/674/699, 130, 145, 145/255/287/391/472/476/557, 145/255/287/391/476/513, 145/255/287/391/557/685/743/758, 145/255/391/398/472/476/513/557/602/660, 145/255/391/398/472/557/685, 145/255/391/398/476/557/758, 145/255/391/398/513/658/685/743, 145/255/391/472/476/685/743, 145/255/391/472/557/685, 145/255/391/476/557/602/660/743, 145/255/391/476/557/658/660/743, 145/255/391/476/758, 145/255/391/513/557/743/758, 145/255/391/557/758, 145/255/398/472/476/513/557/658/660, 145/255/472/476/557/685/743/758, 145/255/472/513/557/602/660/743, 145/255/513, 145/255/513/602, 145/255/557, 145/255/557/658/660/685, 145/255/743/758, 145/267/391/398/513/557/658/685/743, 145/287/391/472/557/602/658/660/685, 145/287/391/472/602/658, 145/287/391/476/513/557/602, 145/287/391/476/658/660/685, 145/287/658/758, 145/391, 145/391/398/476/758, 145/391/472/476/513/557/658/685/758, 145/391/472/476/557/602/660/685/743, 145/391/472/476/557/743/758, 145/391/472/476/658/660/758, 145/391/472/476/658/685/758, 145/391/472/476/758, 145/391/513/557/602, 145/391/513/557/658/685, 145/391/513/658/743/758, 145/391/513/758, 145/391/557, 145/391/557/658/758, 145/391/557/660/685, 145/391/557/743, 145/398/472/557/660, 145/476/557/660/685/743/758, 145/513/685, 145/557/658, 145/557/685/758, 145/685, 161/162, 161/162/170/194/235/423/699, 161/162/170/235/249/252, 161/162/194/269/605/660, 161/162/269/476/660, 161/162/352/434/605, 161/162/659/660/674, 161/170/235/249/252/434, 161/170/235/269/319, 161/170/235/319/412, 161/170/252/352/412/434/699, 161/170/476, 161/194/198/235/252/423/434/605/645/699, 161/194/235/249/352/412/434/674/699, 161/194/252, 161/194/252/323/352/412/605/659/660/674/699, 161/194/252/412/605, 161/194/434, 161/235/249/319/412/605/659, 161/235/252/319/352/645/674/699, 161/235/352/423/645/674, 161/235/645/699, 161/249/412/476/605/645/659/660/674, 161/252/319/352/412/423/476/605/645, 161/252/319/674, 161/269/434/699, 161/319/660, 161/352, 161/423/645, 161/605, 162, 162/170/194/235/249/269/674/699, 162/170/412/434/476/605, 162/194/249/645/659, 162/235/269/423/476/605/645/659, 162/252/352/412/434/645/660/699, 162/269/352/412/476/605, 162/352, 162/423/645, 162/605/645, 164, 168, 170, 170/194/198/235/674/699, 170/194/235/252/269/323/412/423/476/605/645/660/674, 170/194/235/319/412/476/699, 170/194/269/319/412/423/476/605/645, 170/194/319/352/476/699, 170/235/249/352/645, 170/235/269/352, 170/235/269/434/674, 170/235/319/352/412/434/476/645/660/674, 170/235/352/674/699, 170/235/352/699, 170/235/412, 170/235/645, 170/235/660, 170/249/252/269/352/412/423/434/476/659, 170/249/252/269/423/659, 170/249/412, 170/252/352/423/674/699, 170/269/352/423/476/605/659/660/699, 170/269/476/645, 170/412/423/476/605/645/660/674, 170/476/674, 175, 177, 194, 194/198/235/252/323/412/605/645, 194/198/252/319/423/645/674, 194/235/252, 194/235/252/319/423, 194/235/605, 194/235/645, 194/235/699, 194/249/252/645/699, 194/249/412/476/605/645/699, 194/252/269/319/434/605/645/659/674/699, 194/252/319/412/605, 194/269/319/412/605/674/699, 194/319/352/605/660, 194/323/699, 194/352/476/659, 194/352/605/645/699, 194/434, 194/645/659, 197, 198/674/699, 202/268, 217, 227, 235, 235/249/252/323/352/476, 235/249/252/412/605/645/699, 235/249/252/423/699, 235/249/252/434/605/645/699, 235/252, 235/252/269/352/434/605/674/699, 235/252/269/352/476/645/674, 235/252/269/423/605/645/660/674, 235/269/323/412/434/659/674/699, 235/269/674/699, 235/319/605/645/674, 235/352, 235/352/412/659/660/674/699, 235/352/605, 235/412/645, 235/476/674, 235/605/645, 249/252/319/352/645, 249/252/319/605/660, 249/252/412/423/645/674/699, 249/412/476/605, 252/352/645/660, 252/645, 253, 255/260/398/557, 255/287, 255/287/391/472/557/658/660, 255/287/391/472/658/660/685, 255/287/391/476/557, 255/287/391/658, 255/391, 255/391/398/476/557, 255/391/398/476/602/658/660/685, 255/391/398/513/557/658/743/758, 255/391/472/513/758, 255/391/472/557/658/660/685/743/758, 255/391/472/557/658/660/758, 255/391/472/658/660/758, 255/391/472/685, 255/391/476/513/557/602/685/758, 255/391/476/557/602/660/758, 255/391/476/557/658, 255/391/476/758, 255/391/513/602/658/660, 255/391/557, 255/391/602, 255/391/685/758, 255/391/743, 255/398/472/476/557/658, 255/398/476, 255/398/476/557/658/660/685, 255/398/513/758, 255/472/758, 255/513/557, 255/513/557/685/758, 255/557/658, 255/602, 255/660, 267, 268, 269/352, 269/352/412, 269/605, 270, 271/472, 279, 281, 287/391/472, 287/391/472/476/557/602/658/660/685/743, 287/391/476/513, 287/391/513/557/660/685/743/758, 287/391/557, 287/391/557/602/658/743, 287/391/557/685, 287/391/602, 287/685, 297, 319, 319/352/412/434/699, 319/434/476, 322, 329, 331, 339, 347, 350, 352, 352/412/605/645/699, 352/605/645/699, 352/605/659, 352/645, 354, 356, 358, 360, 361, 365, 372, 377, 379, 391, 391/398/476/685, 391/398/513/602/658/660/758, 391/451/472/513/557, 391/472/476/557/602, 391/472/513/557/602/658, 391/472/513/602/658, 391/476/513/743, 391/476/557, 391/513/658/660, 391/557, 391/557/758, 391/602, 391/602/758, 391/658, 391/660/743/758, 391/685, 396, 398, 398/472, 398/557/602/658, 412, 412/645, 412/674, 423, 423/476/605, 423/476/645/674, 423/645, 423/699, 425, 426, 435, 440, 455, 456, 467, 472, 472/476/513/743, 472/476/557/658/758, 472/513/557/658/743/758, 472/513/557/660/685, 472/513/658/660/743, 472/557/660/685/743/758, 472/758, 476, 476/557/758, 476/685, 476/758, 478/557, 479, 485, 506, 508, 513, 513/557/743, 513/557/758, 532, 533, 540, 557, 557/658/660, 557/658/685, 557/743, 564, 602, 602/685, 602/703, 605/699, 635, 643, 645, 645/659, 645/674/699, 658, 658/660, 658/685/758, 659, 660, 674, 704, 713, 718, 758, and 810, or combinations thereof, wherein the amino acid positions are relative to the reference sequence of SEQ ID NO: 2.

In some embodiments, the engineered DNA polymerase comprises one or more substitutions or substitution sets selected from 11D/287L, 15S/633N, 18H/145H/255V, 20C/602H, 29N, 50H/255V/557R/685A, 110M, 113F/170L/194V/235E/249R/319V/645K/659P/660P/674V/699V, 130M, 145H, 145H/255V/287L/391S/472A/476L/557R, 145H/255V/287L/391S/476L/513T, 145H/255V/287L/391S/557R/685A/743T/758W, 145H/255V/391S/398R/472A/476L/513T/557R/602H/660S, 145H/255V/391S/398R/472A/557R/685A, 145H/255V/391S/398R/476L/557R/758W, 145H/255V/391S/398R/513T/658V/685A/743T, 145H/255V/391S/472A/476L/685A/743T, 145H/255V/391S/472A/557R/685A, 145H/255V/391S/476L/557R/602H/660S/743T, 145H/255V/391S/476L/557R/658V/660S/743T, 145H/255V/391S/476L/758W, 145H/255V/391S/513T/557R/743T/758W, 145H/255V/391S/557R/758W, 145H/255V/398R/472A/476L/513T/557R/658V/660S, 145H/255V/472A/476L/557R/685A/743T/758W, 145H/255V/472A/513T/557R/602H/660S/743T, 145H/255V/513T, 145H/255V/513T/602H, 145H/255V/557R, 145H/255V/557R/658V/660S/685A, 145H/255V/743T/758W, 145H/267S/391S/398R/513T/557R/658V/685A/743T, 145H/287L/391S/472A/557R/602H/658V/660S/685A, 145H/287L/391S/472A/602H/658V, 145H/287L/391S/476L/513T/557R/602H, 145H/287L/391S/476L/658V/660S/685A, 145H/287L/658V/758W, 145H/391S, 145H/391S/398R/476L/758W, 145H/391S/472A/476L/513T/557R/658V/685A/758W, 145H/391S/472A/476L/557R/602H/660S/685A/743T, 145H/391S/472A/476L/557R/743T/758W, 145H/391S/472A/476L/658V/660S/758W, 145H/391S/472A/476L/658V/685A/758W, 145H/391S/472A/476L/758W, 145H/391S/513T/557R/602H, 145H/391S/513T/557R/658V/685A, 145H/391S/513T/658V/743T/758W, 145H/391S/513T/758W, 145H/391S/557R, 145H/391S/557R/658V/758W, 145H/391S/557R/660S/685A, 145H/391S/557R/743T, 145H/398R/472A/557R/660S, 145H/476L/557R/660S/685A/743T/758W, 145H/513T/685A, 145H/557R/658V, 145H/557R/685A/758W, 145H/685A, 161V/162A, 161V/162A/170L/194V/235E/423Q/699V, 161V/162A/170L/235E/249R/252L, 161V/162A/194V/269R/605R/660P, 161V/162A/269R/476I/660P, 161V/162A/352K/434K/605R, 161V/162A/659P/660P/674V, 161V/170L/235E/249R/252L/434K, 161V/170L/235E/269R/319V, 161V/170L/235E/319V/412A, 161V/170L/252L/352K/412A/434K/699V, 161V/170L/476I, 161V/194V/198G/235E/252L/423Q/434K/605R/645K/699V, 161V/194V/235E/249R/352K/412A/434K/674V/699V, 161V/194V/252L, 161V/194V/252L/323S/352K/412A/605R/659P/660P/674V/699V, 161V/194V/252L/412A/605R, 161V/194V/434K, 161V/235E/249R/319V/412A/605R/659P, 161V/235E/252L/319V/352K/645K/674V/699V, 161V/235E/352K/423Q/645K/674V, 161V/235E/645K/699V, 161V/249R/412A/476I/605R/645K/659P/660P/674V, 161V/252L/319V/352K/412A/423Q/4761/605R/645K, 161V/252L/319V/674V, 161V/269R/434K/699V, 161V/319V/660P, 161V/352K, 161V/423Q/645K, 161V/605R, 162A, 162A/170L/194V/235E/249R/269R/674V/699V, 162A/170L/412A/434K/476I/605R, 162A/194V/249R/645K/659P, 162A/235E/269R/423Q/476I/605R/645K/659P, 162A/252L/352K/412A/434K/645K/660P/699V, 162A/269R/352K/412A/476I/605R, 162A/352K, 162A/423Q/645K, 162A/605R/645K, 162L, 164C, 168V, 170L, 170L/194V/198G/235E/674V/699V, 170L/194V/235E/252L/269R/323S/412A/423Q/476I/605R/645K/660P/674V, 170L/194V/235E/319V/412A/476I/699V, 170L/194V/269R/319V/412A/423Q/4761/605R/645K, 170L/194V/319V/352K/476I/699V, 170L/235E/249R/352K/645K, 170L/235E/269R/352K, 170L/235E/269R/434K/674V, 170L/235E/319V/352K/412A/434K/476I/645K/660P/674V, 170L/235E/352K/674V/699V, 170L/235E/352K/699V, 170L/235E/412A, 170L/235E/645K, 170L/235E/660P, 170L/249R/252L/269R/352K/412A/423Q/434K/476I/659P, 170L/249R/252L/269R/423Q/659P, 170L/249R/412A, 170L/252L/352K/423Q/674V/699V, 170L/269R/352K/423Q/4761/605R/659P/660P/699V, 170L/269R/4761/645K, 170L/412A/423Q/476I/605R/645K/660P/674V, 170L/4761/674V, 175A, 177F, 194V, 194V/198G/235E/252L/323S/412A/605R/645K, 194V/198G/252L/319V/423Q/645K/674V, 194V/235E/252L, 194V/235E/252L/319V/423Q, 194V/235E/605R, 194V/235E/645K, 194V/235E/699V, 194V/249R/252L/645K/699V, 194V/249R/412A/476I/605R/645K/699V, 194V/252L/269R/319V/434K/605R/645K/659P/674V/699V, 194V/252L/319V/412A/605R, 194V/269R/319V/412A/605R/674V/699V, 194V/319V/352K/605R/660P, 194V/323S/699V, 194V/352K/476I/659P, 194V/352K/605R/645K/699V, 194V/434K, 194V/645K/659P, 197Q, 198G/674V/699V, 202S/268K, 217T, 227E, 235E, 235E/249R/252L/323S/352K/476I, 235E/249R/252L/412A/605R/645K/699V, 235E/249R/252L/423Q/699V, 235E/249R/252L/434K/605R/645K/699V, 235E/252L, 235E/252L/269R/352K/434K/605R/674V/699V, 235E/252L/269R/352K/476I/645K/674V, 235E/252L/269R/423Q/605R/645K/660P/674V, 235E/269R/323S/412A/434K/659P/674V/699V, 235E/269R/674V/699V, 235E/319V/605R/645K/674V, 235E/352K, 235E/352K/412A/659P/660P/674V/699V, 235E/352K/605R, 235E/412A/645K, 235E/476I/674V, 235E/605R/645K, 249R/252L/319V/352K/645K, 249R/252L/319V/605R/660P, 249R/252L/412A/423Q/645K/674V/699V, 249R/412A/476I/605R, 252L/352K/645K/660P, 252L/645K, 253D, 255V/260A/398R/557R, 255V/287L, 255V/287L/391S/472A/557R/658V/660S, 255V/287L/391S/472A/658V/660S/685A, 255V/287L/391S/476L/557R, 255V/287L/391S/658V, 255V/391S, 255V/391S/398R/476L/557R, 255V/391S/398R/476L/602H/658V/660S/685A, 255V/391S/398R/513T/557R/658V/743T/758W, 255V/391S/472A/513T/758W, 255V/391S/472A/557R/658V/660S/685A/743T/758W, 255V/391S/472A/557R/658V/660S/758W, 255V/391S/472A/658V/660S/758W, 255V/391S/472A/685A, 255V/391S/476L/513T/557R/602H/685A/758W, 255V/391S/476L/557R/602H/660S/758W, 255V/391S/476L/557R/658V, 255V/391S/476L/758W, 255V/391S/513T/602H/658V/660S, 255V/391S/557R, 255V/391S/602H, 255V/391S/685A/758W, 255V/391S/743T, 255V/398R/472A/476L/557R/658V, 255V/398R/476L, 255V/398R/476L/557R/658V/660S/685A, 255V/398R/513T/758W, 255V/472A/758W, 255V/513T/557R, 255V/513T/557R/685A/758W, 255V/557R/658V, 255V/602H, 255V/660S, 267Y, 268K, 269R/352K, 269R/352K/412A, 269R/605R, 270Y, 271H/472A, 2791, 281M, 287L/391S/472A, 287L/391S/472A/476L/557R/602H/658V/660S/685A/743T, 287L/391S/476L/513T, 287L/391S/513T/557R/660S/685A/743T/758W, 287L/391S/557R, 287L/391S/557R/602H/658V/743T, 287L/391S/557R/685A, 287L/391S/602H, 287L/685A, 297T, 319V, 319V/352K/412A/434K/699V, 319V/434K/476I, 322V, 329Q, 331S, 339E, 3471, 350F, 352G, 352K, 352K/412A/605R/645K/699V, 352K/605R/645K/699V, 352K/605R/659P, 352K/645K, 354I, 356N, 358N, 360F, 3611, 3651, 372N, 377Q, 3791, 391S, 391S/398R/476L/685A, 391S/398R/513T/602H/658V/660S/758W, 391S/451V/472A/513T/557R, 391S/472A/476L/557R/602H, 391S/472A/513T/557R/602H/658V, 391S/472A/513T/602H/658V, 391S/476L/513T/743T, 391S/476L/557R, 391S/513T/658V/660S, 391S/557R, 391S/557R/758W, 391S/602H, 391S/602H/758W, 391S/658V, 391S/660S/743T/758W, 391S/685A, 396K, 398R, 398R/472A, 398R/557R/602H/658V, 398T, 412A, 412A/645K, 412A/674V, 423Q, 423Q/4761/605R, 423Q/4761/645K/674V, 423Q/645K, 423Q/699V, 423V, 425F, 426V, 435Y, 440T, 455V, 456K, 467N, 472A, 472A/476L/513T/743T, 472A/476L/557R/658V/758W, 472A/513T/557R/658V/743T/758W, 472A/513T/557R/660S/685A, 472A/513T/658V/660S/743T, 472A/557R/660S/685A/743T/758W, 472A/758W, 476I, 476L, 476L/557R/758W, 476L/685A, 476L/758W, 478H/557R, 479M, 485K, 506N, 508N, 513T, 513T/557R/743T, 513T/557R/758W, 532M, 533D, 540N, 557R, 557R/658V/660S, 557R/658V/685A, 557R/743T, 564E, 602H, 602H/685A, 602H/703R, 605R/699V, 635K, 643H, 645K, 645K/659P, 645K/674V/699V, 658V, 658V/660S, 658V/685A/758W, 659E, 660S, 674V, 704K, 713Q, 718D, 758W, and 810K, or combinations thereof, wherein the amino acid positions are relative to SEQ ID NO: 2.

In some embodiments, the engineered DNA polymerase comprises one or more substitutions or substitution sets selected from G11D/F287L, L15S/G633N, P18H/Q145H/S255V, G20C/K602H, H29N, Q50H/S255V/P557R/H685A, L110M, L113F/R170L/A194V/A235E/K249R/R319V/R645K/S659P/A660P/1674V/A699V, L130M, Q145H, Q145H/S255V/F287L/P391S/V472A/M476L/P557R, Q145H/S255V/F287L/P391S/M476L/G513T, Q145H/S255V/F287L/P391S/P557R/H685A/N743T/F758W, Q145H/S255V/P391S/G398R/V472A/M476L/G513T/P557R/K602H/A660S, Q145H/S255V/P391S/G398R/V472A/P557R/H685A, Q145H/S255V/P391S/G398R/M476L/P557R/F758W, Q145H/S255V/P391S/G398R/G513T/L658V/H685A/N743T, Q145H/S255V/P391S/V472A/M476L/H685A/N743T, Q145H/S255V/P391S/V472A/P557R/H685A, Q145H/S255V/P391S/M476L/P557R/K602H/A660S/N743T, Q145H/S255V/P391S/M476L/P557R/L658V/A660S/N743T, Q145H/S255V/P391S/M476L/F758W, Q145H/S255V/P391S/G513T/P557R/N743T/F758W, Q145H/S255V/P391S/P557R/F758W, Q145H/S255V/G398R/V472A/M476L/G513T/P557R/L658V/A660S, Q145H/S255V/V472A/M476L/P557R/H685A/N743T/F758W, Q145H/S255V/V472A/G513T/P557R/K602H/A660S/N743T, Q145H/S255V/G513T, Q145H/S255V/G513T/K602H, Q145H/S255V/P557R, Q145H/S255V/P557R/L658V/A660S/H685A, Q145H/S255V/N743T/F758W, Q145H/F267S/P391S/G398R/G513T/P557R/L658V/H685A/N743T, Q145H/F287L/P391S/V472A/P557R/K602H/L658V/A660S/H685A, Q145H/F287L/P391S/V472A/K602H/L658V, Q145H/F287L/P391S/M476L/G513T/P557R/K602H, Q145H/F287L/P391S/M476L/L658V/A660S/H685A, Q145H/F287L/L658V/F758W, Q145H/P391S, Q145H/P391S/G398R/M476L/F758W, Q145H/P391S/V472A/M476L/G513T/P557R/L658V/H685A/F758W, Q145H/P391S/V472A/M476L/P557R/K602H/A660S/H685A/N743T, Q145H/P391S/V472A/M476L/P557R/N743T/F758W, Q145H/P391S/V472A/M476L/L658V/A660S/F758W, Q145H/P391S/V472A/M476L/L658V/H685A/F758W, Q145H/P391S/V472A/M476L/F758W, Q145H/P391S/G513T/P557R/K602H, Q145H/P391S/G513T/P557R/L658V/H685A, Q145H/P391S/G513T/L658V/N743T/F758W, Q145H/P391S/G513T/F758W, Q145H/P391S/P557R, Q145H/P391S/P557R/L658V/F758W, Q145H/P391S/P557R/A660S/H685A, Q145H/P391S/P557R/N743T, Q145H/G398R/V472A/P557R/A660S, Q145H/M476L/P557R/A660S/H685A/N743T/F758W, Q145H/G513T/H685A, Q145H/P557R/L658V, Q145H/P557R/H685A/F758W, Q145H/H685A, I161V/S162A, I161V/S162A/R170L/A194V/A235E/A423Q/A699V, I161V/S162A/R170L/A235E/K249R/R252L, I161V/S162A/A194V/G269R/K605R/A660P, I161V/S162A/G269R/M476I/A660P, I161V/S162A/R352K/R434K/K605R, I161V/S162A/S659P/A660P/1674V, I161V/R170L/A235E/K249R/R252L/R434K, I161V/R170L/A235E/G269R/R319V, I161V/R170L/A235E/R319V/G412A, I161V/R170L/R252L/R352K/G412A/R434K/A699V, I161V/R170L/M476I, I161V/A194V/S198G/A235E/R252L/A423Q/R434K/K605R/R645K/A699V, I161V/A194V/A235E/K249R/R352K/G412A/R434K/1674V/A699V, I161V/A194V/R252L, I161V/A194V/R252L/P323S/R352K/G412A/K605R/S659P/A660P/1674V/A699V, I161V/A194V/R252L/G412A/K605R, I161V/A194V/R434K, I161V/A235E/K249R/R319V/G412A/K605R/S659P, I161V/A235E/R252L/R319V/R352K/R645K/1674V/A699V, I161V/A235E/R352K/A423Q/R645K/1674V, I161V/A235E/R645K/A699V, I161V/K249R/G412A/M476I/K605R/R645K/S659P/A660P/1674V, I161V/R252L/R319V/R352K/G412A/A423Q/M476I/K605R/R645K, I161V/R252L/R319V/1674V, I161V/G269R/R434K/A699V, I161V/R319V/A660P, 1161V/R352K, I161V/A423Q/R645K, I161V/K605R, S162A, S162A/R170L/A194V/A235E/K249R/G269R/1674V/A699V, S162A/R170L/G412A/R434K/M476I/K605R, S162A/A194V/K249R/R645K/S659P, S162A/A235E/G269R/A423Q/M476I/K605R/R645K/S659P, S162A/R252L/R352K/G412A/R434K/R645K/A660P/A699V, S162A/G269R/R352K/G412A/M476I/K605R, S162A/R352K, S162A/A423Q/R645K, S162A/K605R/R645K, S162L, L164C, G168V, R170L, R170L/A194V/S198G/A235E/1674V/A699V, R170L/A194V/A235E/R252L/G269R/P323S/G412A/A423Q/M476I/K605R/R645K/A660P/1674V, R170L/A194V/A235E/R319V/G412A/M476I/A699V, R170L/A194V/G269R/R319V/G412A/A423Q/M476I/K605R/R645K, R170L/A194V/R319V/R352K/M476I/A699V, R170L/A235E/K249R/R352K/R645K, R170L/A235E/G269R/R352K, R170L/A235E/G269R/R434K/1674V, R170L/A235E/R319V/R352K/G412A/R434K/M476I/R645K/A660P/1674V, R170L/A235E/R352K/1674V/A699V, R170L/A235E/R352K/A699V, R170L/A235E/G412A, R170L/A235E/R645K, R170L/A235E/A660P, R170L/K249R/R252L/G269R/R352K/G412A/A423Q/R434K/M476I/S659P, R170L/K249R/R252L/G269R/A423Q/S659P, R170L/K249R/G412A, R170L/R252L/R352K/A423Q/1674V/A699V, R170L/G269R/R352K/A423Q/M476I/K605R/S659P/A660P/A699V, R170L/G269R/M476I/R645K, R170L/G412A/A423Q/M476I/K605R/R645K/A660P/1674V, R170L/M476I/1674V, W175A, W177F, A194V, A194V/S198G/A235E/R252L/P323S/G412A/K605R/R645K, A194V/S198G/R252L/R319V/A423Q/R645K/1674V, A194V/A235E/R252L, A194V/A235E/R252L/R319V/A423Q, A194V/A235E/K605R, A194V/A235E/R645K, A194V/A235E/A699V, A194V/K249R/R252L/R645K/A699V, A194V/K249R/G412A/M476I/K605R/R645K/A699V, A194V/R252L/G269R/R319V/R434K/K605R/R645K/S659P/1674V/A699V, A194V/R252L/R319V/G412A/K605R, A194V/G269R/R319V/G412A/K605R/1674V/A699V, A194V/R319V/R352K/K605R/A660P, A194V/P323S/A699V, A194V/R352K/M476I/S659P, A194V/R352K/K605R/R645K/A699V, A194V/R434K, A194V/R645K/S659P, P197Q, S198G/1674V/A699V, P202S/R268K, K217T, K227E, A235E, A235E/K249R/R252L/P323S/R352K/M476I, A235E/K249R/R252L/G412A/K605R/R645K/A699V, A235E/K249R/R252L/A423Q/A699V, A235E/K249R/R252L/R434K/K605R/R645K/A699V, A235E/R252L, A235E/R252L/G269R/R352K/R434K/K605R/1674V/A699V, A235E/R252L/G269R/R352K/M476I/R645K/1674V, A235E/R252L/G269R/A423Q/K605R/R645K/A660P/1674V, A235E/G269R/P323S/G412A/R434K/S659P/1674V/A699V, A235E/G269R/1674V/A699V, A235E/R319V/K605R/R645K/1674V, A235E/R352K, A235E/R352K/G412A/S659P/A660P/1674V/A699V, A235E/R352K/K605R, A235E/G412A/R645K, A235E/M476I/1674V, A235E/K605R/R645K, K249R/R252L/R319V/R352K/R645K, K249R/R252L/R319V/K605R/A660P, K249R/R252L/G412A/A423Q/R645K/1674V/A699V, K249R/G412A/M476I/K605R, R252L/R352K/R645K/A660P, R252L/R645K, E253D, S255V/D260A/G398R/P557R, S255V/F287L, S255V/F287L/P391S/V472A/P557R/L658V/A660S, S255V/F287L/P391S/V472A/L658V/A660S/H685A, S255V/F287L/P391S/M476L/P557R, S255V/F287L/P391S/L658V, S255V/P391S, S255V/P391S/G398R/M476L/P557R, S255V/P391S/G398R/M476L/K602H/L658V/A660S/H685A, S255V/P391S/G398R/G513T/P557R/L658V/N743T/F758W, S255V/P391S/V472A/G513T/F758W, S255V/P391S/V472A/P557R/L658V/A660S/H685A/N743T/F758W, S255V/P391S/V472A/P557R/L658V/A660S/F758W, S255V/P391S/V472A/L658V/A660S/F758W, S255V/P391S/V472A/H685A, S255V/P391S/M476L/G513T/P557R/K602H/H685A/F758W, S255V/P391S/M476L/P557R/K602H/A660S/F758W, S255V/P391S/M476L/P557R/L658V, S255V/P391S/M476L/F758W, S255V/P391S/G513T/K602H/L658V/A660S, S255V/P391S/P557R, S255V/P391S/K602H, S255V/P391S/H685A/F758W, S255V/P391S/N743T, S255V/G398R/V472A/M476L/P557R/L658V, S255V/G398R/M476L, S255V/G398R/M476L/P557R/L658V/A660S/H685A, S255V/G398R/G513T/F758W, S255V/V472A/F758W, S255V/G513T/P557R, S255V/G513T/P557R/H685A/F758W, S255V/P557R/L658V, S255V/K602H, S255V/A660S, F267Y, R268K, G269R/R352K, G269R/R352K/G412A, G269R/K605R, R270Y, R271H/V472A, K279I, F281M, F287L/P391S/V472A, F287L/P391S/V472A/M476L/P557R/K602H/L658V/A660S/H685A/N743T, F287L/P391S/M476L/G513T, F287L/P391S/G513T/P557R/A660S/H685A/N743T/F758W, F287L/P391S/P557R, F287L/P391S/P557R/K602H/L658V/N743T, F287L/P391S/P557R/H685A, F287L/P391S/K602H, F287L/H685A, L297T, R319V, R319V/R352K/G412A/R434K/A699V, R319V/R434K/M476I, R322V, E329Q, L331S, G339E, P347I, A350F, R352G, R352K, R352K/G412A/K605R/R645K/A699V, R352K/K605R/R645K/A699V, R352K/K605R/S659P, R352K/R645K, L354I, E356N, R358N, L360F, L361I, L365I, E372N, P377Q, T379I, P391S, P391S/G398R/M476L/H685A, P391S/G398R/G513T/K602H/L658V/A660S/F758W, P391S/A451V/V472A/G513T/P557R, P391S/V472A/M476L/P557R/K602H, P391S/V472A/G513T/P557R/K602H/L658V, P391S/V472A/G513T/K602H/L658V, P391S/M476L/G513T/N743T, P391S/M476L/P557R, P391S/G513T/L658V/A660S, P391S/P557R, P391S/P557R/F758W, P391S/K602H, P391S/K602H/F758W, P391S/L658V, P391S/A660S/N743T/F758W, P391S/H685A, P396K, G398R, G398R/V472A, G398R/P557R/K602H/L658V, G398T, G412A, G412A/R645K, G412A/1674V, A423Q, A423Q/M476I/K605R, A423Q/M476I/R645K/1674V, A423Q/R645K, A423Q/A699V, A423V, L425F, L426V, L435Y, E440T, A455V, T456K, A467N, V472A, V472A/M476L/G513T/N743T, V472A/M476L/P557R/L658V/F758W, V472A/G513T/P557R/L658V/N743T/F758W, V472A/G513T/P557R/A660S/H685A, V472A/G513T/L658V/A660S/N743T, V472A/P557R/A660S/H685A/N743T/F758W, V472A/F758W, M476I, M476L, M476L/P557R/F758W, M476L/H685A, M476L/F758W, R478H/P557R, L479M, R485K, E506N, G508N, G513T, G513T/P557R/N743T, G513T/P557R/F758W, R532M, E533D, K540N, P557R, P557R/L658V/A660S, P557R/L658V/H685A, P557R/N743T, P564E, K602H, K602H/H685A, K602H/Q703R, K605R/A699V, E635K, E643H, R645K, R645K/S659P, R645K/1674V/A699V, L658V, L658V/A660S, L658V/H685A/F758W, S659E, A660S, 1674V, R704K, K713Q, K718D, F758W, and A810K, or combinations thereof, wherein the amino acid positions are relative to the reference sequence of SEQ ID NO: 2.

In some embodiments, the engineered DNA polymerase comprises at least one or more substitutions or substitution sets at amino acid positions selected from 11/287, 29, 50/255/557/685, 145/255/287/391/472/476/557, 145/255/391/472/557/685, 145/255/391/685, 145/255/472/476/557/685/743/758, 145/255/557/658/660/685, 145/391/472/476/513/557/658/685/758, 145/391/513/557/602, 145/391/557/743, 145/398/472/557/660, 145/557/658, 145/743, 161/162/269/476/660, 161/162/659/660/674, 161/170/235/269/319, 161/170/235/319/412, 161/194/235/249/352/412/434/674/699, 161/194/252/412/605, 161/235/252/319/352/645/674/699, 161/235/645/699, 161/249/412/476/605/645/659/660/674, 161/252/319/352/412/423/476/605/645, 161/319/660, 161/352, 162, 162/352, 162/423/645, 170, 170/194/235/252/269/323/412/423/476/605/645/660/674, 170/235/249/352/645, 170/235/319/352/412/434/476/645/660/674, 170/235/352/674/699, 170/235/352/699, 170/235/412, 170/235/645, 170/235/660, 170/252/352/423/674/699, 170/269/352/423/476/605/659/660/699, 194, 194/198/252/319/423/645/674, 194/235/252, 194/252/269/319/434/605/645/659/674/699, 194/269/319/412/605/674/699, 194/319/352/605/660, 194/352/605/645/699, 194/645/659, 197, 217, 227, 235/249/252/323/352/476, 235/249/252/423/699, 235/252, 235/252/269/423/605/645/660/674, 235/269/323/412/434/659/674/699, 235/352, 235/352/605, 235/605/645, 249/252/319/605/660, 252/645, 255, 255/391/472/557/660/743, 255/391/476/513/557/602/685/758, 255/391/476/557/658, 255/391/557, 255/398/472/476/557/658, 255/398/476/557/658/660/685, 255/476, 255/660, 268, 269/352, 269/352/412, 269/605, 319, 319/352/412/434/699, 319/434/476, 329, 331, 339, 352, 352/412/605/645/699, 354, 358, 360, 391, 391/398/476/685, 391/451/472/513/557, 391/472/476/557/602, 391/472/476/557/658/660/743/758, 391/557, 391/581, 412, 412/645, 412/674, 423/476/645/674, 423/645, 467, 478/557, 506, 540, 557, 557/658/660, 557/658/685, 602/701, 602/703, 605/699, 658, 658/660, 660, 699, 713, and 743, wherein the amino acid positions are relative to the reference sequence of SEQ ID NO: 2.

In some embodiments, the engineered DNA polymerase comprises one or more substitutions or substitution sets selected from 11D/287L, 29N, 50H/255V/557R/685A, 145H/255V/287L/391S/472A/476L/557R, 145H/255V/391S/472A/557R/685A, 145H/255V/391S/685A, 145H/255V/472A/476L/557R/685A/743T/758W, 145H/255V/557R/658V/660S/685A, 145H/391S/472A/476L/513T/557R/658V/685A/758W, 145H/391S/513T/557R/602H, 145H/391S/557R/743T, 145H/398R/472A/557R/660S, 145H/557R/658V, 145H/743T, 161V/162A/269R/4761/660P, 161V/162A/659P/660P/674V, 161V/170L/235E/269R/319V, 161V/170L/235E/319V/412A, 161V/194V/235E/249R/352K/412A/434K/674V/699V, 161V/194V/252L/412A/605R, 161V/235E/252L/319V/352K/645K/674V/699V, 161V/235E/645K/699V, 161V/249R/412A/476I/605R/645K/659P/660P/674V, 161V/252L/319V/352K/412A/423Q/4761/605R/645K, 161V/319V/660P, 161V/352K, 162A, 162A/352K, 162A/423Q/645K, 170L, 170L/194V/235E/252L/269R/323S/412A/423Q/476I/605R/645K/660P/674V, 170L/235E/249R/352K/645K, 170L/235E/319V/352K/412A/434K/476I/645K/660P/674V, 170L/235E/352K/674V/699V, 170L/235E/352K/699V, 170L/235E/412A, 170L/235E/645K, 170L/235E/660P, 170L/252L/352K/423Q/674V/699V, 170L/269R/352K/423Q/4761/605R/659P/660P/699V, 194V, 194V/198G/252L/319V/423Q/645K/674V, 194V/235E/252L, 194V/252L/269R/319V/434K/605R/645K/659P/674V/699V, 194V/269R/319V/412A/605R/674V/699V, 194V/319V/352K/605R/660P, 194V/352K/605R/645K/699V, 194V/645K/659P, 197Q, 217T, 227E, 235E/249R/252L/323S/352K/476I, 235E/249R/252L/423Q/699V, 235E/252L, 235E/252L/269R/423Q/605R/645K/660P/674V, 235E/269R/323S/412A/434K/659P/674V/699V, 235E/352K, 235E/352K/605R, 235E/605R/645K, 249R/252L/319V/605R/660P, 252L/645K, 255V, 255V/391S/472A/557R/660S/743T, 255V/391S/476L/513T/557R/602H/685A/758W, 255V/391S/476L/557R/658V, 255V/391S/557R, 255V/398R/472A/476L/557R/658V, 255V/398R/476L/557R/658V/660S/685A, 255V/476L, 255V/660S, 268K, 269R/352K, 269R/352K/412A, 269R/605R, 319V, 319V/352K/412A/434K/699V, 319V/434K/476I, 329Q, 331S, 339E, 352G, 352K, 352K/412A/605R/645K/699V, 3541, 358N, 360F, 391S, 391S/398R/476L/685A, 391S/451V/472A/513T/557R, 391S/472A/476L/557R/602H, 391S/472A/476L/557R/658V/660S/743T/758W, 391S/557R, 391S/581D, 412A, 412A/645K, 412A/674V, 423Q/4761/645K/674V, 423Q/645K, 467N, 478H/557R, 506N, 540N, 557R, 557R/658V/660S, 557R/658V/685A, 602H/701L, 602H/703R, 605R/699V, 658V, 658V/660S, 660S, 699V, 713Q, and 743T, wherein the amino acid positions are relative to the reference sequence of SEQ ID NO: 2.

In some embodiments, the engineered DNA polymerase comprises one or more substitutions or substitution sets selected from G11D/F287L, H29N, Q50H/S255V/P557R/H685A, Q145H/S255V/F287L/P391S/V472A/M476L/P557R, Q145H/S255V/P391S/V472A/P557R/H685A, Q145H/S255V/P391S/H685A, Q145H/S255V/V472A/M476L/P557R/H685A/N743T/F758W, Q145H/S255V/P557R/L658V/A660S/H685A, Q145H/P391S/V472A/M476L/G513T/P557R/L658V/H685A/F758W, Q145H/P391S/G513T/P557R/K602H, Q145H/P391S/P557R/N743T, Q145H/G398R/V472A/P557R/A660S, Q145H/P557R/L658V, Q145H/N743T, I161V/S162A/G269R/M476I/A660P, I161V/S162A/S659P/A660P/1674V, I161V/R170L/A235E/G269R/R319V, I161V/R170L/A235E/R319V/G412A, I161V/A194V/A235E/K249R/R352K/G412A/R434K/1674V/A699V, I161V/A194V/R252L/G412A/K605R, I161V/A235E/R252L/R319V/R352K/R645K/1674V/A699V, I161V/A235E/R645K/A699V, I161V/K249R/G412A/M476I/K605R/R645K/S659P/A660P/1674V, I161V/R252L/R319V/R352K/G412A/A423Q/M476I/K605R/R645K, I161V/R319V/A660P, I161V/R352K, S162A, S162A/R352K, S162A/A423Q/R645K, R170L, R170L/A194V/A235E/R252L/G269R/P323S/G412A/A423Q/M476I/K605R/R645K/A660P/1674V, R170L/A235E/K249R/R352K/R645K, R170L/A235E/R319V/R352K/G412A/R434K/M476I/R645K/A660P/1674V, R170L/A235E/R352K/I674V/A699V, R170L/A235E/R352K/A699V, R170L/A235E/G412A, R170L/A235E/R645K, R170L/A235E/A660P, R170L/R252L/R352K/A423Q/1674V/A699V, R170L/G269R/R352K/A423Q/M476I/K605R/S659P/A660P/A699V, A194V, A194V/S198G/R252L/R319V/A423Q/R645K/1674V, A194V/A235E/R252L, A194V/R252L/G269R/R319V/R434K/K605R/R645K/S659P/1674V/A699V, A194V/G269R/R319V/G412A/K605R/1674V/A699V, A194V/R319V/R352K/K605R/A660P, A194V/R352K/K605R/R645K/A699V, A194V/R645K/S659P, P197Q, K217T, K227E, A235E/K249R/R252L/P323S/R352K/M476I, A235E/K249R/R252L/A423Q/A699V, A235E/R252L, A235E/R252L/G269R/A423Q/K605R/R645K/A660P/1674V, A235E/G269R/P323S/G412A/R434K/S659P/1674V/A699V, A235E/R352K, A235E/R352K/K605R, A235E/K605R/R645K, K249R/R252L/R319V/K605R/A660P, R252L/R645K, S255V, S255V/P391S/V472A/P557R/A660S/N743T, S255V/P391S/M476L/G513T/P557R/K602H/H685A/F758W, S255V/P391S/M476L/P557R/L658V, S255V/P391S/P557R, S255V/G398R/V472A/M476L/P557R/L658V, S255V/G398R/M476L/P557R/L658V/A660S/H685A, S255V/M476L, S255V/A660S, R268K, G269R/R352K, G269R/R352K/G412A, G269R/K605R, R319V, R319V/R352K/G412A/R434K/A699V, R319V/R434K/M476I, E329Q, L331S, G339E, R352G, R352K, R352K/G412A/K605R/R645K/A699V, L354I, R358N, L360F, P391S, P391S/G398R/M476L/H685A, P391S/A451V/V472A/G513T/P557R, P391S/V472A/M476L/P557R/K602H, P391S/V472A/M476L/P557R/L658V/A660S/N743T/F758W, P391S/P557R, P391S/G581D, G412A, G412A/R645K, G412A/1674V, A423Q/M476I/R645K/1674V, A423Q/R645K, A467N, R478H/P557R, E506N, K540N, P557R, P557R/L658V/A660S, P557R/L658V/H685A, K602H/F701L, K602H/Q703R, K605R/A699V, L658V, L658V/A660S, A660S, A699V, K713Q, and N743T, wherein the amino acid positions are relative to the reference sequence of SEQ ID NO: 2.

In some additional embodiments, the present invention provides an engineered DNA polymerase or a functional fragment thereof, comprising a polypeptide sequence having at least 75% or more sequence identity to a reference sequence comprising residues 12 to 841 of SEQ ID NO: 178, or to a reference sequence of SEQ ID NO: 178, wherein the polypeptide sequence comprises one or more substitutions relative to the reference sequence comprising residues 12 to 841 of SEQ ID NO: 178, or to the reference sequence of SEQ ID NO: 178.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 178 or to the reference sequence of SEQ ID NO: 178.

In some embodiments, the engineered DNA polymerase comprises one or more substitutions or substitution sets at amino acid positions selected from 12, 14, 15, 17, 19, 36, 39, 44, 45, 82, 86, 87, 95, 122, 139, 139/395, 159, 167, 170/194, 170/194/423/451/557/658/660/743, 170/194/423/476/743, 170/194/423/557/658/659/743, 170/194/423/557/658/660/685, 170/194/423/557/658/743, 170/194/451/476/557/658, 170/194/451/476/557/658/659/660/743, 170/194/451/476/557/658/685, 170/194/451/557/659/660, 170/194/476/557/659, 170/194/557, 170/194/658, 170/194/658/659/660/685/743, 170/194/658/743, 170/423, 170/423/451/476/557/658/659/743, 170/423/451/557, 170/423/451/557/658/659/685/743, 170/423/451/557/659, 170/423/476, 170/423/476/658/659, 170/423/476/658/659/660/685/743, 170/423/476/658/660/685/743, 170/423/476/658/685, 170/423/476/658/685/743, 170/423/476/685, 170/423/476/685/743, 170/423/557/658/659, 170/423/557/658/660/743, 170/423/557/658/743, 170/423/557/659/660/685/743, 170/423/557/660/743, 170/423/557/685, 170/423/557/685/743, 170/423/658, 170/423/658/659/743, 170/423/658/660, 170/423/743, 170/451/557, 170/451/557/658/660, 170/476/557/658/659, 170/476/557/658/685, 170/476/557/743, 170/476/659, 170/557, 170/557/658/660/685/687, 170/658, 170/658/659, 170/658/659/743, 170/659, 170/660, 179, 194/423/451/557/658/659/743, 194/423/476/658/660/685, 194/423/557/658/659/743, 194/423/685, 194/451/476/557/658/659, 194/451/476/557/658/659/660/743, 194/557, 194/557/658/659/685, 194/557/659/743, 194/658/659/685, 194/658/660, 197, 198, 198/423/476/557/743, 205, 209, 214, 222, 228, 249, 250/269/331, 252, 252/258/331, 252/258/331/352/467/506/674, 252/269, 252/269/331/339/412/413, 252/269/331/352/412/674/704, 252/269/331/506/674, 252/269/339/352/356/660/704, 252/269/339/352/412/699/704, 252/269/352/660, 252/269/412/674, 252/269/413/467/660/699/704, 252/269/467/506, 252/269/674, 252/331, 252/331/339/352/467/506, 252/331/339/412/413/467/699, 252/331/352/412/413/467/660/704, 252/331/352/412/506/674, 252/331/358/506, 252/331/412/413, 252/331/412/413/674, 252/331/412/467/506/699/704, 252/331/412/506/674, 252/331/412/506/704, 252/331/413, 252/331/467/660/674, 252/331/660, 252/331/699, 252/339/352/467/674/699, 252/339/352/674/704, 252/339/412/413/467/674, 252/339/412/413/674/699/704, 252/339/412/467/674, 252/339/412/674/704, 252/339/467/506/699/704, 252/341/412/413, 252/341/412/674, 252/352/412, 252/352/506, 252/352/660, 252/358/674, 252/412, 252/412/413, 252/412/467, 252/412/467/660/699/704, 252/413, 252/413/506/660/704, 252/413/699, 252/467, 252/467/506/674/704, 252/506/704, 252/674, 252/699, 252/704, 264, 268, 268/510, 268/621, 269, 269/331/352/412/467/506/704, 269/331/352/467/506/699, 269/331/352/467/699, 269/331/412/413/467/506/660/674, 269/331/413/506, 269/331/467/506/660, 269/331/660/699, 269/331/674/699, 269/331/699, 269/331/699/704, 269/339/506/704, 269/352/467/506/674, 269/358, 269/358/412/660/704, 269/358/467/506/660/699, 269/412/413/506/704, 269/412/413/674, 269/412/467/704, 269/467/506, 269/506, 269/599/660/674/699, 269/674, 269/699/704, 269/704, 272/352/506, 275, 301, 323, 331, 331/339/412/413/467/506/674/704, 331/339/412/467/506/704, 331/352/358/699/704, 331/352/412/413/506, 331/358, 331/358/467/674/699/704, 331/412, 331/412/413/467/699/704, 331/412/467/674/704, 331/412/467/704, 331/412/660/699, 331/413/467/660/674, 331/467, 331/467/506, 331/467/674/699, 331/506, 331/506/674/699, 331/660/674/699, 331/674, 331/674/699, 331/699, 331/704, 339, 339/352, 339/352/412/419/467/506/660/674/699, 339/352/412/467/660, 339/352/412/660/674, 339/352/674/699, 339/412/413, 339/412/413/467/674/699, 339/412/467/674, 339/467/674, 339/506, 339/506/699, 339/506/704, 339/660, 339/660/674, 341/352/704, 341/467, 341/467/506/660/674, 341/467/674, 345, 352, 352/358, 352/412/413/467/506/699, 352/413/467/506, 352/413/674, 352/660, 352/674, 355, 358/412, 358/413, 358/674, 412, 412/413/467/660, 412/413/467/674, 412/467/506/674/704, 412/467/660/674/699, 412/660, 412/674, 412/674/699, 413/467, 413/506, 413/506/704, 413/674/699/704/740, 423, 423/451/476/557/658/685, 423/451/476/658/685, 423/451/557, 423/451/557/658, 423/451/557/658/685, 423/476, 423/476/557/658/659/660, 423/476/557/658/659/685/743, 423/476/557/659/660/685/743, 423/476/557/659/660/743, 423/476/658/660/743, 423/476/658/685/743, 423/476/658/743, 423/557/658, 423/557/658/659/660, 423/557/658/659/660/685, 423/557/658/660/743, 423/557/685, 423/557/685/743, 423/658, 423/658/659/660, 423/658/660, 423/660, 423/685, 423/685/743, 423/743, 428, 433, 433/497, 467, 467/506/699/704, 467/658/660/674/699, 470, 476, 476/480/557/659, 476/557/658/659, 476/557/658/660/685, 476/557/659, 476/557/659/660, 476/658/659/660/685/743, 476/658/660/743, 477, 478, 485, 489/557/743, 492, 501, 506/660, 506/674/699/704, 506/674/704, 511, 514, 517, 520, 525/557/659/660/743, 533, 534, 534/670, 549, 557, 557/658, 557/658/659, 557/658/659/660, 557/659/660, 557/660, 557/743, 565, 596, 598, 642, 658, 658/659, 658/659/660/685/743, 658/659/685, 658/743, 659/660, 659/660/685/743, 674, 674/699, 685, 689, 695, 696, 699, 699/704, 704, 707, 725, 743, 745, 747, 783, and 821, or combinations thereof, wherein the amino acid positions are relative to the reference sequence of SEQ ID NO: 178.

In some embodiments, the engineered DNA polymerase comprises one or more substitutions or substitution sets selected from 12E, 12L, 12S, 12V, 14E, 14V, 15V, 17S, 19A, 19S, 36H, 39E, 39P, 39V, 44A, 44L, 45L, 45S, 82K, 86G, 87A, 87P, 87R, 95D, 95G, 95R, 122A, 122Q, 139D/395A, 139S, 159Q, 167D, 170L/194V, 170L/194V/423Q/451H/557R/658V/660S/743T, 170L/194V/423Q/476L/743T, 170L/194V/423Q/557R/658V/659P/743T, 170L/194V/423Q/557R/658V/660S/685A, 170L/194V/423Q/557R/658V/743T, 170L/194V/451H/476L/557R/658V, 170L/194V/451H/476L/557R/658V/659P/660S/743T, 170L/194V/451H/476L/557R/658V/685A, 170L/194V/451H/557R/659P/660S, 170L/194V/476L/557R/659P, 170L/194V/557R, 170L/194V/658V, 170L/194V/658V/659P/660S/685A/743T, 170L/194V/658V/743T, 170L/423Q, 170L/423Q/451H/476L/557R/658V/659P/743T, 170L/423Q/451H/557R, 170L/423Q/451H/557R/658V/659P/685A/743T, 170L/423Q/451H/557R/659P, 170L/423Q/476L, 170L/423Q/476L/658V/659P, 170L/423Q/476L/658V/659P/660S/685A/743T, 170L/423Q/476L/658V/660S/685A/743T, 170L/423Q/476L/658V/685A, 170L/423Q/476L/658V/685A/743T, 170L/423Q/476L/685A, 170L/423Q/476L/685A/743T, 170L/423Q/557R/658V/659P, 170L/423Q/557R/658V/660S/743T, 170L/423Q/557R/658V/743T, 170L/423Q/557R/659P/660S/685A/743T, 170L/423Q/557R/660S/743T, 170L/423Q/557R/685A, 170L/423Q/557R/685A/743T, 170L/423Q/658V, 170L/423Q/658V/659P/743T, 170L/423Q/658V/660S, 170L/423Q/743T, 170L/451H/557R, 170L/451H/557R/658V/660S, 170L/476L/557R/658V/659P, 170L/476L/557R/658V/685A, 170L/476L/557R/743T, 170L/476L/659P, 170L/557R, 170L/557R/658V/660S/685A/687F, 170L/658V, 170L/658V/659P, 170L/658V/659P/743T, 170L/659P, 170L/660S, 179M, 194V/423Q/451H/557R/658V/659P/743T, 194V/423Q/476L/658V/660S/685A, 194V/423Q/557R/658V/659P/743T, 194V/423Q/685A, 194V/451H/476L/557R/658V/659P, 194V/451H/476L/557R/658V/659P/660S/743T, 194V/557R, 194V/557R/658V/659P/685A, 194V/557R/659P/743T, 194V/658V/659P/685A, 194V/658V/660S, 197K, 198G, 198G/423Q/476L/557R/743T, 198H, 198P, 198V, 205R, 205S, 209P, 209R, 209S, 209V, 209W, 214D, 214R, 2221, 228S, 249E, 2501/269R/331S, 252L, 252L/258H/331S, 252L/258H/331S/352K/467N/506N/674V, 252L/269R, 252L/269R/331S/339E/412A/413K, 252L/269R/331S/352K/412A/674V/704K, 252L/269R/331S/506N/674V, 252L/269R/339E/352K/356G/660P/704K, 252L/269R/339E/352K/412A/699V/704K, 252L/269R/352K/660P, 252L/269R/412A/674V, 252L/269R/413K/467N/660P/699V/704K, 252L/269R/467N/506N, 252L/269R/674V, 252L/331S, 252L/331S/339E/352K/467N/506N, 252L/331S/339E/412A/413K/467N/699V, 252L/331S/352K/412A/413K/467N/660P/704K, 252L/331S/352K/412A/506N/674V, 252L/331S/358N/506N, 252L/331S/412A/413K, 252L/331S/412A/413K/674V, 252L/331S/412A/467N/506N/699V/704K, 252L/331S/412A/506N/674V, 252L/331S/412A/506N/704K, 252L/331S/413K, 252L/331S/467N/660P/674V, 252L/331S/660P, 252L/331S/699V, 252L/339E/352K/467N/674V/699V, 252L/339E/352K/674V/704K, 252L/339E/412A/413K/467N/674V, 252L/339E/412A/413K/674V/699V/704K, 252L/339E/412A/467N/674V, 252L/339E/412A/674V/704K, 252L/339E/467N/506N/699V/704K, 252L/341A/412A/413K, 252L/341A/412A/674V, 252L/352K/412A, 252L/352K/506N, 252L/352K/660P, 252L/358N/674V, 252L/412A, 252L/412A/413K, 252L/412A/467N, 252L/412A/467N/660P/699V/704K, 252L/413K, 252L/413K/506N/660P/704K, 252L/413K/699V, 252L/467N, 252L/467N/506N/674V/704K, 252L/506N/704K, 252L/674V, 252L/699V, 252L/704K, 264A, 264S, 268A/621N, 268G, 268H/510A, 269R, 269R/331S/352K/412A/467N/506N/704K, 269R/331S/352K/467N/506N/699V, 269R/331S/352K/467N/699V, 269R/331S/412A/413K/467N/506N/660P/674V, 269R/331S/413K/506N, 269R/331S/467N/506N/660P, 269R/331S/660P/699V, 269R/331S/674V/699V, 269R/331S/699V, 269R/331S/699V/704K, 269R/339E/506N/704K, 269R/352K/467N/506N/674V, 269R/358N, 269R/358N/412A/660P/704K, 269R/358N/467N/506N/660P/699V, 269R/412A/413K/506N/704K, 269R/412A/413K/674V, 269R/412A/467N/704K, 269R/467N/506N, 269R/506N, 269R/599F/660P/674V/699V, 269R/674V, 269R/699V/704K, 269R/704K, 272S/352K/506N, 275E, 275F, 275L, 275M, 301G, 301M, 301T, 323L, 323Q, 331S, 331S/339E/412A/413K/467N/506N/674V/704K, 331S/339E/412A/467N/506N/704K, 331S/352K/358N/699V/704K, 331S/352K/412A/413K/506N, 331S/358N, 331S/358N/467N/674V/699V/704K, 331S/412A, 331S/412A/413K/467N/699V/704K, 331S/412A/467N/674V/704K, 331S/412A/467N/704K, 331S/412A/660P/699V, 331S/413K/467N/660P/674V, 331S/467N, 331S/467N/506N, 331S/467N/674V/699V, 331S/506N, 331S/506N/674V/699V, 331S/660P/674V/699V, 331S/674V, 331S/674V/699V, 331S/699V, 331S/704K, 339E, 339E/352K, 339E/352K/412A/419K/467N/506N/660P/674V/699V, 339E/352K/412A/467N/660P, 339E/352K/412A/660P/674V, 339E/352K/674V/699V, 339E/412A/413K, 339E/412A/413K/467N/674V/699V, 339E/412A/467N/674V, 339E/467N/674V, 339E/506N, 339E/506N/699V, 339E/506N/704K, 339E/660P, 339E/660P/674V, 341A/352K/704K, 341A/467N, 341A/467N/506N/660P/674V, 341A/467N/674V, 345P, 352G, 352K/358N, 352K/412A/413K/467N/506N/699V, 352K/413K/467N/506N, 352K/413K/674V, 352K/660P, 352K/674V, 355G, 358N/412A, 358N/413K, 358N/674V, 412A, 412A/413K/467N/660P, 412A/413K/467N/674V, 412A/467N/506N/674V/704K, 412A/467N/660P/674V/699V, 412A/660P, 412A/674V, 412A/674V/699V, 413K/467N, 413K/506N, 413K/506N/704K, 413K/674V/699V/704K/740H, 423Q, 423Q/451H/476L/557R/658V/685A, 423Q/451H/476L/658V/685A, 423Q/451H/557R, 423Q/451H/557R/658V, 423Q/451H/557R/658V/685A, 423Q/476L, 423Q/476L/557R/658V/659P/660S, 423Q/476L/557R/658V/659P/685A/743T, 423Q/476L/557R/659P/660S/685A/743T, 423Q/476L/557R/659P/660S/743T, 423Q/476L/658V/660S/743T, 423Q/476L/658V/685A/743T, 423Q/476L/658V/743T, 423Q/557R/658V, 423Q/557R/658V/659P/660S, 423Q/557R/658V/659P/660S/685A, 423Q/557R/658V/660S/743T, 423Q/557R/685A, 423Q/557R/685A/743T, 423Q/658V, 423Q/658V/659P/660S, 423Q/658V/660S, 423Q/660S, 423Q/685A, 423Q/685A/743T, 423Q/743T, 428C, 428G, 428Q, 433G, 433K, 433P/497N, 433R, 433S, 433V, 467N, 467N/506N/699V/704K, 467N/658V/660S/674V/699V, 470E, 470G, 470H, 470V, 476L, 476L/480K/557R/659P, 476L/557R/658V/659P, 476L/557R/658V/660S/685A, 476L/557R/659P, 476L/557R/659P/660S, 476L/658V/659P/660S/685A/743T, 476L/658V/660S/743T, 477E, 477T, 478G, 478I, 478L, 478S, 485A, 485E, 485G, 485I, 485M, 485T, 485V, 489Q/557R/743T, 492M, 492R, 492Y, 501K, 501L, 506N/660P, 506N/674V/699V/704K, 506N/674V/704K, 511L, 514G, 514V, 517A, 517H, 517L, 517M, 517R, 517T, 520V, 525V/557R/659P/660S/743T, 533L, 533P, 534A, 534A/670V, 549V, 557R, 557R/658V, 557R/658V/659P, 557R/658V/659P/660S, 557R/659P/660S, 557R/660S, 557R/743T, 565R, 565S, 565Y, 596S, 598G, 598H, 598K, 598L, 598R, 642E, 658V, 658V/659P, 658V/659P/660S/685A/743T, 658V/659P/685A, 658V/743T, 659P/660S, 659P/660S/685A/743T, 674V, 674V/699V, 685A, 689D, 689N, 695K, 695Q, 695T, 696A, 696G, 696K, 696Q, 699V, 699V/704K, 704K, 707A, 707R, 707S, 725H, 743T, 745P, 747V, 783H, and 821S, or combinations thereof, wherein the amino acid positions are relative to the reference sequence of SEQ ID NO: 178.

In some embodiments, the engineered DNA polymerase comprises one or more substitutions or substitution sets selected from M12E, M12L, M12S, M12V, P14E, P14V, L15V, E17S, K19A, K19S, F36H, K39E, K39P, K39V, S44A, S44L, R45L, R45S, R82K, Y86G, E87A, E87P, E87R, P95D, P95G, P95R, P122A, P122Q, R139D/T395A, R139S, P159Q, E167D, R170L/A194V, R170L/A194V/A423Q/A451H/P557R/L658V/A660S/N743T, R170L/A194V/A423Q/M476L/N743T, R170L/A194V/A423Q/P557R/L658V/S659P/N743T, R170L/A194V/A423Q/P557R/L658V/A660S/H685A, R170L/A194V/A423Q/P557R/L658V/N743T, R170L/A194V/A451H/M476L/P557R/L658V, R170L/A194V/A451H/M476L/P557R/L658V/S659P/A660S/N743T, R170L/A194V/A451H/M476L/P557R/L658V/H685A, R170L/A194V/A451H/P557R/S659P/A660S, R170L/A194V/M476L/P557R/S659P, R170L/A194V/P557R, R170L/A194V/L658V, R170L/A194V/L658V/S659P/A660S/H685A/N743T, R170L/A194V/L658V/N743T, R170L/A423Q, R170L/A423Q/A451H/M476L/P557R/L658V/S659P/N743T, R170L/A423Q/A451H/P557R, R170L/A423Q/A451H/P557R/L658V/S659P/H685A/N743T, R170L/A423Q/A451H/P557R/S659P, R170L/A423Q/M476L, R170L/A423Q/M476L/L658V/S659P, R170L/A423Q/M476L/L658V/S659P/A660S/H685A/N743T, R170L/A423Q/M476L/L658V/A660S/H685A/N743T, R170L/A423Q/M476L/L658V/H685A, R170L/A423Q/M476L/L658V/H685A/N743T, R170L/A423Q/M476L/H685A, R170L/A423Q/M476L/H685A/N743T, R170L/A423Q/P557R/L658V/S659P, R170L/A423Q/P557R/L658V/A660S/N743T, R170L/A423Q/P557R/L658V/N743T, R170L/A423Q/P557R/S659P/A660S/H685A/N743T, R170L/A423Q/P557R/A660S/N743T, R170L/A423Q/P557R/H685A, R170L/A423Q/P557R/H685A/N743T, R170L/A423Q/L658V, R170L/A423Q/L658V/S659P/N743T, R170L/A423Q/L658V/A660S, R170L/A423Q/N743T, R170L/A451H/P557R, R170L/A451H/P557R/L658V/A660S, R170L/M476L/P557R/L658V/S659P, R170L/M476L/P557R/L658V/H685A, R170L/M476L/P557R/N743T, R170L/M476L/S659P, R170L/P557R, R170L/P557R/L658V/A660S/H685A/L687F, R170L/L658V, R170L/L658V/S659P, R170L/L658V/S659P/N743T, R170L/S659P, R170L/A660S, K179M, A194V/A423Q/A451H/P557R/L658V/S659P/N743T, A194V/A423Q/M476L/L658V/A660S/H685A, A194V/A423Q/P557R/L658V/S659P/N743T, A194V/A423Q/H685A, A194V/A451H/M476L/P557R/L658V/S659P, A194V/A451H/M476L/P557R/L658V/S659P/A660S/N743T, A194V/P557R, A194V/P557R/L658V/S659P/H685A, A194V/P557R/S659P/N743T, A194V/L658V/S659P/H685A, A194V/L658V/A660S, P197K, S198G, S198G/A423Q/M476L/P557R/N743T, S198H, S198P, S198V, K205R, K205S, E209P, E209R, E209S, E209V, E209W, K214D, K214R, L222I, N228S, K249E, L250I/G269R/L331S, R252L, R252L/R258H/L331S, R252L/R258H/L331S/R352K/A467N/E506N/1674V, R252L/G269R, R252L/G269R/L331S/G339E/G412A/E413K, R252L/G269R/L331S/R352K/G412A/1674V/R704K, R252L/G269R/L331S/E506N/1674V, R252L/G269R/G339E/R352K/E356G/A660P/R704K, R252L/G269R/G339E/R352K/G412A/A699V/R704K, R252L/G269R/R352K/A660P, R252L/G269R/G412A/1674V, R252L/G269R/E413K/A467N/A660P/A699V/R704K, R252L/G269R/A467N/E506N, R252L/G269R/1674V, R252L/L331S, R252L/L331S/G339E/R352K/A467N/E506N, R252L/L331S/G339E/G412A/E413K/A467N/A699V, R252L/L331S/R352K/G412A/E413K/A467N/A660P/R704K, R252L/L331S/R352K/G412A/E506N/1674V, R252L/L331S/R358N/E506N, R252L/L331S/G412A/E413K, R252L/L331S/G412A/E413K/1674V, R252L/L331S/G412A/A467N/E506N/A699V/R704K, R252L/L331S/G412A/E506N/1674V, R252L/L331S/G412A/E506N/R704K, R252L/L331S/E413K, R252L/L331S/A467N/A660P/1674V, R252L/L331S/A660P, R252L/L331S/A699V, R252L/G339E/R352K/A467N/1674V/A699V, R252L/G339E/R352K/I674V/R704K, R252L/G339E/G412A/E413K/A467N/1674V, R252L/G339E/G412A/E413K/1674V/A699V/R704K, R252L/G339E/G412A/A467N/1674V, R252L/G339E/G412A/1674V/R704K, R252L/G339E/A467N/E506N/A699V/R704K, R252L/V341A/G412A/E413K, R252L/V341A/G412A/1674V, R252L/R352K/G412A, R252L/R352K/E506N, R252L/R352K/A660P, R252L/R358N/1674V, R252L/G412A, R252L/G412A/E413K, R252L/G412A/A467N, R252L/G412A/A467N/A660P/A699V/R704K, R252L/E413K, R252L/E413K/E506N/A660P/R704K, R252L/E413K/A699V, R252L/A467N, R252L/A467N/E506N/1674V/R704K, R252L/E506N/R704K, R252L/1674V, R252L/A699V, R252L/R704K, E264A, E264S, R268A/S621N, R268G, R268H/P510A, G269R, G269R/L331S/R352K/G412A/A467N/E506N/R704K, G269R/L331S/R352K/A467N/E506N/A699V, G269R/L331S/R352K/A467N/A699V, G269R/L331S/G412A/E413K/A467N/E506N/A660P/1674V, G269R/L331S/E413K/E506N, G269R/L331S/A467N/E506N/A660P, G269R/L331S/A660P/A699V, G269R/L331S/1674V/A699V, G269R/L331S/A699V, G269R/L331S/A699V/R704K, G269R/G339E/E506N/R704K, G269R/R352K/A467N/E506N/1674V, G269R/R358N, G269R/R358N/G412A/A660P/R704K, G269R/R358N/A467N/E506N/A660P/A699V, G269R/G412A/E413K/E506N/R704K, G269R/G412A/E413K/I674V, G269R/G412A/A467N/R704K, G269R/A467N/E506N, G269R/E506N, G269R/L599F/A660P/1674V/A699V, G269R/1674V, G269R/A699V/R704K, G269R/R704K, E272S/R352K/E506N, W275E, W275F, W275L, W275M, L301G, L301M, L301T, P323L, P323Q, L331S, L331S/G339E/G412A/E413K/A467N/E506N/1674V/R704K, L331S/G339E/G412A/A467N/E506N/R704K, L331S/R352K/R358N/A699V/R704K, L331S/R352K/G412A/E413K/E506N, L331S/R358N, L331S/R358N/A467N/I674V/A699V/R704K, L331S/G412A, L331S/G412A/E413K/A467N/A699V/R704K, L331S/G412A/A467N/1674V/R704K, L331S/G412A/A467N/R704K, L331S/G412A/A660P/A699V, L331S/E413K/A467N/A660P/1674V, L331S/A467N, L331S/A467N/E506N, L331S/A467N/1674V/A699V, L331S/E506N, L331S/E506N/1674V/A699V, L331S/A660P/1674V/A699V, L331S/1674V, L331S/1674V/A699V, L331S/A699V, L331S/R704K, G339E, G339E/R352K, G339E/R352K/G412A/E419K/A467N/E506N/A660P/1674V/A699V, G339E/R352K/G412A/A467N/A660P, G339E/R352K/G412A/A660P/1674V, G339E/R352K/1674V/A699V, G339E/G412A/E413K, G339E/G412A/E413K/A467N/1674V/A699V, G339E/G412A/A467N/1674V, G339E/A467N/1674V, G339E/E506N, G339E/E506N/A699V, G339E/E506N/R704K, G339E/A660P, G339E/A660P/1674V, V341A/R352K/R704K, V341A/A467N, V341A/A467N/E506N/A660P/1674V, V341A/A467N/1674V, E345P, R352G, R352K/R358N, R352K/G412A/E413K/A467N/E506N/A699V, R352K/E413K/A467N/E506N, R352K/E413K/1674V, R352K/A660P, R352K/1674V, K355G, R358N/G412A, R358N/E413K, R358N/1674V, G412A, G412A/E413K/A467N/A660P, G412A/E413K/A467N/1674V, G412A/A467N/E506N/1674V/R704K, G412A/A467N/A660P/1674V/A699V, G412A/A660P, G412A/1674V, G412A/1674V/A699V, E413K/A467N, E413K/E506N, E413K/E506N/R704K, E413K/1674V/A699V/R704K/P740H, A423Q, A423Q/A451H/M476L/P557R/L658V/H685A, A423Q/A451H/M476L/L658V/H685A, A423Q/A451H/P557R, A423Q/A451H/P557R/L658V, A423Q/A451H/P557R/L658V/H685A, A423Q/M476L, A423Q/M476L/P557R/L658V/S659P/A660S, A423Q/M476L/P557R/L658V/S659P/H685A/N743T, A423Q/M476L/P557R/S659P/A660S/H685A/N743T, A423Q/M476L/P557R/S659P/A660S/N743T, A423Q/M476L/L658V/A660S/N743T, A423Q/M476L/L658V/H685A/N743T, A423Q/M476L/L658V/N743T, A423Q/P557R/L658V, A423Q/P557R/L658V/S659P/A660S, A423Q/P557R/L658V/S659P/A660S/H685A, A423Q/P557R/L658V/A660S/N743T, A423Q/P557R/H685A, A423Q/P557R/H685A/N743T, A423Q/L658V, A423Q/L658V/S659P/A660S, A423Q/L658V/A660S, A423Q/A660S, A423Q/H685A, A423Q/H685A/N743T, A423Q/N743T, R428C, R428G, R428Q, E433G, E433K, E433P/D497N, E433R, E433S, E433V, A467N, A467N/E506N/A699V/R704K, A467N/L658V/A660S/1674V/A699V, L470E, L470G, L470H, L470V, M476L, M476L/E480K/P557R/S659P, M476L/P557R/L658V/S659P, M476L/P557R/L658V/A660S/H685A, M476L/P557R/S659P, M476L/P557R/S659P/A660S, M476L/L658V/S659P/A660S/H685A/N743T, M476L/L658V/A660S/N743T, R477E, R477T, R478G, R478I, R478L, R478S, R485A, R485E, R485G, R485I, R485M, R485T, R485V, H489Q/P557R/N743T, N492M, N492R, N492Y, R501K, R501L, E506N/A660P, E506N/1674V/A699V/R704K, E506N/1674V/R704K, P511L, K514G, K514V, K517A, K517H, K517L, K517M, K517R, K517T, K520V, A525V/P557R/S659P/A660S/N743T, E533L, E533P, V534A, V534A/A670V, K549V, P557R, P557R/L658V, P557R/L658V/S659P, P557R/L658V/S659P/A660S, P557R/S659P/A660S, P557R/A660S, P557R/N743T, K565R, K565S, K565Y, R596S, P598G, P598H, P598K, P598L, P598R, Q642E, L658V, L658V/S659P, L658V/S659P/A660S/H685A/N743T, L658V/S659P/H685A, L658V/N743T, S659P/A660S, S659P/A660S/H685A/N743T, 1674V, 1674V/A699V, H685A, Q689D, Q689N, Y695K, Y695Q, Y695T, E696A, E696G, E696K, E696Q, A699V, A699V/R704K, R704K, Q707A, Q707R, Q707S, T725H, N743T, R745P, K747V, E783H, and P821S, or combinations thereof, wherein the amino acid positions are relative to the reference sequence of SEQ ID NO: 178.

In some embodiments, the engineered DNA polymerase comprises one or more substitutions or substitution sets at amino acid positions selected from 12, 13, 14, 15, 17, 19/214, 45, 84, 87, 95, 122, 159, 170/194/476/557/659, 170/423/476/658/685, 170/423/557/658/743, 170/476/557/658/659, 170/476/557/743, 178, 194/658/660, 198, 209, 214, 222, 228, 249, 252, 252/258/331, 252/269, 252/269/412/674, 252/269/674, 252/331, 252/331/352/412/506/674, 252/331/467/660/674, 252/331/660, 252/331/699, 252/352/412, 252/412, 252/412/467/660/699/704, 252/413, 252/467/506/674/704, 252/704, 264, 268, 268/621, 269/331/699, 269/412/467/704, 269/467/506, 269/674, 269/695, 269/699/704, 275, 276, 301, 323, 331/358, 331/412/660/699, 331/674, 331/704, 345, 352/674, 412/467/660/674/699, 412/660, 412/674, 430, 430/837, 433, 467, 470, 476/557/658/659, 476/658/660/743, 477, 478, 485, 492, 511, 514, 517, 520, 533, 534, 534/670, 549, 557/658/659, 565, 596, 598, 642, 658/659, 674, 689, 695, 696, 699/704, 707, 725, and 783, or combinations thereof, wherein the amino acid positions are relative to the reference sequence of SEQ ID NO: 178.

In some embodiments, the engineered DNA polymerase comprises one or more substitutions or substitution sets selected from 12E, 12V, 13G, 14E, 14V, 15V, 17G, 17K, 17S, 19L/214R, 45S, 84N, 84T, 87A, 87R, 87S, 95D, 95G, 122Q, 159Q, 170L/194V/476L/557R/659P, 170L/423Q/476L/658V/685A, 170L/423Q/557R/658V/743T, 170L/476L/557R/658V/659P, 170L/476L/557R/743T, 178G, 178L, 178N, 178S, 194V/658V/660S, 198V, 209S, 209W, 214R, 2221, 228D, 228S, 249R, 252L, 252L/258H/331S, 252L/269R, 252L/269R/412A/674V, 252L/269R/674V, 252L/331S, 252L/331S/352K/412A/506N/674V, 252L/331S/467N/660P/674V, 252L/331S/660P, 252L/331S/699V, 252L/352K/412A, 252L/412A, 252L/412A/467N/660P/699V/704K, 252L/413K, 252L/467N/506N/674V/704K, 252L/704K, 264T, 268A/621N, 268G, 269C/695R, 269R/331S/699V, 269R/412A/467N/704K, 269R/467N/506N, 269R/674V, 269R/699V/704K, 275E, 275G, 275M, 275V, 276A, 276D, 276G, 276M, 301G, 323L, 323Q, 323S, 331S/358N, 331S/412A/660P/699V, 331S/674V, 331S/704K, 345G, 345P, 352K/674V, 412A/467N/660P/674V/699V, 412A/660P, 412A/674V, 430A/837S, 430F, 433K, 433R, 433S, 433V, 467N, 470M, 470R, 476L/557R/658V/659P, 476L/658V/660S/743T, 477E, 478G, 478L, 478M, 478S, 485A, 485G, 485I, 485T, 485W, 492R, 492Y, 511C, 511L, 514G, 514V, 517A, 517H, 517L, 517R, 517T, 520V, 533L, 533P, 534A, 534A/670V, 549V, 557R/658V/659P, 565R, 596S, 598G, 598K, 598L, 598R, 642E, 642R, 658V/659P, 674V, 689D, 695K, 695S, 695T, 696G, 696K, 696T, 699V/704K, 707A, 725H, and 783H, or combinations thereof, wherein the amino acid positions are relative to the reference sequence of SEQ ID NO: 178.

In some embodiments, the engineered DNA polymerase comprises one or more substitutions or substitution sets selected from M12E, M12V, L13G, P14E, P14V, L15V, E17G, E17K, E17S, K19L/K214R, R45S, E84N, E84T, E87A, E87R, E87S, P95D, P95G, P122Q, P159Q, R170L/A194V/M476L/P557R/S659P, R170L/A423Q/M476L/L658V/H685A, R170L/A423Q/P557R/L658V/N743T, R170L/M476L/P557R/L658V/S659P, R170L/M476L/P557R/N743T, E178G, E178L, E178N, E178S, A194V/L658V/A660S, S198V, E209S, E209W, K214R, L222I, N228D, N228S, K249R, R252L, R252L/R258H/L331S, R252L/G269R, R252L/G269R/G412A/1674V, R252L/G269R/1674V, R252L/L331S, R252L/L331S/R352K/G412A/E506N/1674V, R252L/L331S/A467N/A660P/1674V, R252L/L331S/A660P, R252L/L331S/A699V, R252L/R352K/G412A, R252L/G412A, R252L/G412A/A467N/A660P/A699V/R704K, R252L/E413K, R252L/A467N/E506N/1674V/R704K, R252L/R704K, E264T, R268A/S621N, R268G, G269C/Y695R, G269R/L331S/A699V, G269R/G412A/A467N/R704K, G269R/A467N/E506N, G269R/1674V, G269R/A699V/R704K, W275E, W275G, W275M, W275V, E276A, E276D, E276G, E276M, L301G, P323L, P323Q, P323S, L331S/R358N, L331S/G412A/A660P/A699V, L331S/1674V, L331S/R704K, E345G, E345P, R352K/1674V, G412A/A467N/A660P/1674V/A699V, G412A/A660P, G412A/1674V, Q430A/L837S, Q430F, E433K, E433R, E433S, E433V, A467N, L470M, L470R, M476L/P557R/L658V/S659P, M476L/L658V/A660S/N743T, R477E, R478G, R478L, R478M, R478S, R485A, R485G, R485I, R485T, R485W, N492R, N492Y, P511C, P511L, K514G, K514V, K517A, K517H, K517L, K517R, K517T, K520V, E533L, E533P, V534A, V534A/A670V, K549V, P557R/L658V/S659P, K565R, R596S, P598G, P598K, P598L, P598R, Q642E, Q642R, L658V/S659P, 1674V, Q689D, Y695K, Y695S, Y695T, E696G, E696K, E696T, A699V/R704K, Q707A, T725H, and E783H, or combinations thereof, wherein the amino acid positions are relative to the reference sequence of SEQ ID NO: 178.

In some embodiments, the engineered DNA polymerase comprises one or more substitutions or substitution sets at amino acid positions selected from 12, 13, 14, 15, 17, 84, 87, 122, 159, 170/423/476/658/685, 170/476/557/743, 178, 198, 205, 209, 222, 228, 249, 252, 252/269, 252/269/412/674, 252/269/674, 252/331, 252/331/352/412/506/674, 252/331/467/660/674, 252/331/699, 252/412, 252/412/467/660/699/704, 252/413, 252/467/506/674/704, 252/704, 264, 269/331/699, 269/412/467/704, 269/467/506, 269/674, 275, 276, 301, 322, 323, 331/358, 331/412/660/699, 331/674, 331/704, 345, 352/674, 355, 412/674, 430, 430/837, 433, 467, 470, 476/658/660/743, 477, 478, 485, 492, 511, 514, 517, 520, 533, 534, 534/670, 549, 557/658/659, 565, 596, 598, 642, 674, 689, 695, 696, 699/704, 703, 725, and 821, or combinations thereof, wherein the amino acid positions are relative to the reference sequence of SEQ ID NO: 178.

In some embodiments, the engineered DNA polymerase comprises one or more substitutions or substitution sets selected from 12S, 12V, 13G, 14V, 15V, 17G, 17K, 17S, 84N, 84T, 87A, 87Q, 87R, 87S, 122Q, 159Q, 170L/423Q/476L/658V/685A, 170L/476L/557R/743T, 178G, 178L, 178N, 178S, 198V, 205R, 209S, 2221, 228D, 228S, 249G, 249R, 252L, 252L/269R, 252L/269R/412A/674V, 252L/269R/674V, 252L/331S, 252L/331S/352K/412A/506N/674V, 252L/331S/467N/660P/674V, 252L/331S/699V, 252L/412A, 252L/412A/467N/660P/699V/704K, 252L/413K, 252L/467N/506N/674V/704K, 252L/704K, 264S, 264T, 269R/331S/699V, 269R/412A/467N/704K, 269R/467N/506N, 269R/674V, 275E, 275G, 275V, 276A, 276D, 276G, 276M, 301G, 322L, 323L, 331S/358N, 331S/412A/660P/699V, 331S/674V, 331S/704K, 345G, 352K/674V, 355F, 412A/674V, 430A/837S, 430F, 433R, 433S, 433V, 467N, 470E, 470M, 470R, 476L/658V/660S/743T, 477E, 478G, 478M, 485A, 485E, 485G, 485I, 485W, 492R, 492Y, 511C, 511E, 511L, 514G, 514V, 517A, 517H, 517T, 520V, 533L, 533P, 534A, 534A/670V, 549V, 557R/658V/659P, 565R, 565Y, 596S, 598G, 598L, 642R, 674V, 689D, 695K, 695S, 695T, 696G, 696K, 696T, 696W, 699V/704K, 703K, 725L, and 821R, or combinations thereof, wherein the amino acid positions are relative to the reference sequence of SEQ ID NO: 178.

In some embodiments, the engineered DNA polymerase comprises one or more substitutions or substitution sets selected from M12S, M12V, L13G, P14V, L15V, E17G, E17K, E17S, E84N, E84T, E87A, E87Q, E87R, E87S, P122Q, P159Q, R170L/A423Q/M476L/L658V/H685A, R170L/M476L/P557R/N743T, E178G, E178L, E178N, E178S, S198V, K205R, E209S, L222I, N228D, N228S, K249G, K249R, R252L, R252L/G269R, R252L/G269R/G412A/1674V, R252L/G269R/1674V, R252L/L331S, R252L/L331S/R352K/G412A/E506N/1674V, R252L/L331S/A467N/A660P/1674V, R252L/L331S/A699V, R252L/G412A, R252L/G412A/A467N/A660P/A699V/R704K, R252L/E413K, R252L/A467N/E506N/1674V/R704K, R252L/R704K, E264S, E264T, G269R/L331S/A699V, G269R/G412A/A467N/R704K, G269R/A467N/E506N, G269R/1674V, W275E, W275G, W275V, E276A, E276D, E276G, E276M, L301G, R322L, P323L, L331S/R358N, L331S/G412A/A660P/A699V, L331S/1674V, L331S/R704K, E345G, R352K/1674V, K355F, G412A/1674V, Q430A/L837S, Q430F, E433R, E433S, E433V, A467N, L470E, L470M, L470R, M476L/L658V/A660S/N743T, R477E, R478G, R478M, R485A, R485E, R485G, R485I, R485W, N492R, N492Y, P511C, P511E, P511L, K514G, K514V, K517A, K517H, K517T, K520V, E533L, E533P, V534A, V534A/A670V, K549V, P557R/L658V/S659P, K565R, K565Y, R596S, P598G, P598L, Q642R, 1674V, Q689D, Y695K, Y695S, Y695T, E696G, E696K, E696T, E696W, A699V/R704K, Q703K, T725L, and P821R, or combinations thereof, wherein the amino acid positions are relative to the reference sequence of SEQ ID NO: 178.

In some embodiments, the engineered DNA polymerase comprises one or more substitutions or substitution sets at amino acid positions selected from 12, 15, 17, 252, 252/331, 252/331/352/412/506/674, 252/413, 252/704, 269/412/467/704, 269/467/506, 269/674, 301, 331/704, 345, 352/674, 355, 430/837, 492, 511, 514, 520, 598, 642, 674, 695, 696, 699/704, and 703, or combinations thereof, wherein the amino acid positions are relative to the reference sequence of SEQ ID NO: 178.

In some embodiments, the engineered DNA polymerase comprises one or more substitutions or substitution sets selected from 12V, 15V, 17G, 17K, 252L, 252L/331S, 252L/331S/352K/412A/506N/674V, 252L/413K, 252L/704K, 269R/412A/467N/704K, 269R/467N/506N, 269R/674V, 301G, 331S/704K, 345G, 352K/674V, 355F, 430A/837S, 492R, 492Y, 511C, 511L, 514G, 514V, 520V, 598G, 642R, 674V, 695S, 695T, 696T, 699V/704K, and 703K, or combinations thereof, wherein the amino acid positions are relative to the reference sequence of SEQ ID NO: 178.

In some embodiments, the engineered DNA polymerase comprises one or more substitutions or substitution sets selected from M12V, L15V, E17G, E17K, R252L, R252L/L331S, R252L/L331S/R352K/G412A/E506N/1674V, R252L/E413K, R252L/R704K, G269R/G412A/A467N/R704K, G269R/A467N/E506N, G269R/1674V, L301G, L331S/R704K, E345G, R352K/1674V, K355F, Q430A/L837S, N492R, N492Y, P511C, P511L, K514G, K514V, K520V, P598G, Q642R, 1674V, Y695S, Y695T, E696T, A699V/R704K, and Q703K, or combinations thereof, wherein the amino acid positions are relative to the reference sequence of SEQ ID NO: 178.

The present invention also provides engineered DNA polymerases or a functional fragments thereof, comprising a polypeptide sequence having at least 75% or more sequence identity to a reference sequence comprising residues 12 to 841 of SEQ ID NO: 994, or to a reference sequence of SEQ ID NO: 994, wherein the polypeptide sequence comprises one or more substitutions relative to the reference sequence comprising residues 12 to 841 of SEQ ID NO: 994, or to the reference sequence of SEQ ID NO: 994.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 994 or to the reference sequence of SEQ ID NO: 994.

In some embodiments, the engineered DNA polymerase comprises one or more substitutions or substitution sets at amino acid positions selected from 2, 5, 7, 8, 12/15/269/355/412/413/492/704, 12/252/413/492, 32, 33, 35/304, 38, 40, 65, 80, 81, 140, 151, 169, 194, 194/252, 194/252/358, 194/252/358/412/467, 194/252/358/412/467/658/696/743, 194/252/412/696, 194/252/467/696/743, 194/252/743, 194/358, 194/358/412, 194/358/658, 194/358/704, 194/412, 194/412/467, 194/412/467/743, 194/467/511/658, 194/467/658, 194/467/704, 194/467/743, 194/743, 218, 226, 242, 252, 252/412/658, 252/467/696, 258/347, 279, 284, 289, 298, 300, 304, 311, 329, 331, 340, 358, 375, 375/376, 406, 420, 434, 440, 481, 489, 490, 518, 529, 543, 560, 592, 635, 639, 645, 658, 658/704, 666, 685, 694, 704, 713, 717, 718, 721, 740, 746, 782, 804, 807, 814, and 829, or combinations thereof, wherein the amino acid positions are relative to the reference sequence of SEQ ID NO: 994.

In some embodiments, the engineered DNA polymerase comprises one or more substitutions or substitution sets selected from 2G, 2R, 5A, 5G, 5V, 5W, 7Q, 8R, 12V/15V/269R/355F/412A/413K/492R/704K, 12V/252L/413K/492R, 32L, 33K, 35L/304G, 38E, 38G, 38R, 38S, 38V, 40N, 40R, 40S, 65S, 80G, 81A, 140M, 151K, 169S, 194V, 194V/252L, 194V/252L/358N, 194V/252L/358N/412A/467N, 194V/252L/358N/412A/467N/658V/696T/743T, 194V/252L/412A/696T, 194V/252L/467N/696T/743T, 194V/252L/743T, 194V/358N, 194V/358N/412A, 194V/358N/658V, 194V/358N/704K, 194V/412A, 194V/412A/467N, 194V/412A/467N/743T, 194V/467N/511C/658V, 194V/467N/658V, 194V/467N/704K, 194V/467N/743T, 194V/743T, 218T, 218Y, 226E, 242G, 242N, 252L, 252L/412A/658V, 252L/467N/696T, 258L/347H, 279R, 284A, 289D, 289G, 298A, 298S, 300A, 300F, 300L, 300N, 300R, 300T, 300W, 304C, 304G, 304T, 304V, 311C, 329L, 329S, 331D, 331Q, 331Y, 340T, 358N, 375A/376I, 375I, 406M, 420S, 420T, 434K, 440N, 440T, 481D, 481G, 489R, 490R, 490V, 518L, 518R, 529R, 529T, 529V, 543A, 560G, 560K, 592A, 592S, 635I, 639Q, 645L, 645M, 645R, 658A, 658C, 658E, 658R, 658V/704K, 666D, 666G, 666K, 666M, 666N, 666R, 666S, 666T, 666V, 685T, 694S, 694T, 704Q, 704V, 713V, 717T, 718R, 718S, 721V, 721W, 740G, 746G, 746Q, 746R, 782S, 804D, 804Q, 807H, 814A, 814I, 814R, 829L, and 829R, or combinations thereof, wherein the amino acid positions are relative to the reference sequence of SEQ ID NO: 994.

In some embodiments, the engineered DNA polymerase comprises one or more substitutions or substitution sets selected from H2G, H2R, H5A, H5G, H5V, H5W, H7Q, G8R, M12V/L15V/G269R/K355F/G412A/E413K/Y492R/R704K, M12V/R252L/E413K/Y492R, Y32L, R33K, F35L/E304G, L38E, L38G, L38R, L38S, L38V, G40N, G40R, G40S, E65S, S80G, F81A, E140M, R151K, H169S, A194V, A194V/R252L, A194V/R252L/R358N, A194V/R252L/R358N/G412A/A467N, A194V/R252L/R358N/G412A/A467N/L658V/E696T/N743T, A194V/R252L/G412A/E696T, A194V/R252L/A467N/E696T/N743T, A194V/R252L/N743T, A194V/R358N, A194V/R358N/G412A, A194V/R358N/L658V, A194V/R358N/R704K, A194V/G412A, A194V/G412A/A467N, A194V/G412A/A467N/N743T, A194V/A467N/P511C/L658V, A194V/A467N/L658V, A194V/A467N/R704K, A194V/A467N/N743T, A194V/N743T, E218T, E218Y, L226E, L242G, L242N, R252L, R252L/G412A/L658V, R252L/A467N/E696T, R258L/P347H, K279R, R284A, S289D, S289G, E298A, E298S, P300A, P300F, P300L, P300N, P300R, P300T, P300W, E304C, E304G, E304T, E304V, P311C, E329L, E329S, L331D, L331Q, L331Y, R340T, R358N, D375A/L376I, D375I, E406M, R420S, R420T, R434K, E440N, E440T, E481D, E481G, H489R, S490R, S490V, T518L, T518R, E529R, E529T, E529V, Q543A, A560G, A560K, N592A, N592S, E635I, R639Q, K645L, K645M, K645R, L658A, L658C, L658E, L658R, L658V/R704K, L666D, L666G, L666K, L666M, L666N, L666R, L666S, L666T, L666V, H685T, P694S, P694T, R704Q, R704V, K713V, E717T, K718R, K718S, E721V, E721W, P740G, V746G, V746Q, V746R, R782S, R804D, R804Q, E807H, E814A, E814I, E814R, E829L, and E829, or combinations thereof, wherein the amino acid positions are relative to the reference sequence of SEQ ID NO: 994.

In some embodiments, the engineered DNA polymerase comprises one or more substitutions or substitution sets at amino acid positions selected from 2, 5, 8, 12/15/269/355/412/413/492/704, 12/252/413/492, 38, 40, 65, 80, 140, 151, 218, 226, 230, 242, 258/347, 279, 284, 289, 298, 300, 304, 309, 311, 329, 331, 340, 375, 375/376, 406, 420, 434, 441, 489, 490, 518, 529, 543, 560, 592, 635, 639, 645, 658, 666, 685, 704, 713, 717, 718, 721, 740, 743, 746, 782, 804, 807, 814, and 829, or combinations thereof, wherein the amino acid positions are relative to the reference sequence of SEQ ID NO: 994.

In some embodiments, the engineered DNA polymerase comprises one or more substitutions or substitution sets selected from 2G, 2V, 5A, 5G, 5L, 5V, 5W, 8R, 12V/15V/269R/355F/412A/413K/492R/704K, 12V/252L/413K/492R, 38G, 38R, 38S, 38V, 40R, 40S, 65G, 65H, 65S, 80G, 80M, 140M, 151K, 218Y, 226E, 230G, 242A, 242G, 242R, 258L/347H, 279R, 284A, 284G, 289D, 298A, 298S, 300F, 300N, 300R, 304C, 304G, 304R, 304V, 309G, 311C, 311G, 329L, 331D, 331G, 331Y, 340T, 375A/376I, 375E, 375L, 406G, 420S, 420T, 434K, 441R, 489R, 490R, 490V, 518M, 518R, 529V, 543A, 543L, 543R, 560G, 592A, 592S, 635I, 639Q, 645R, 658Q, 658R, 666G, 666Q, 666R, 666V, 685F, 704V, 713V, 717T, 718R, 721D, 721M, 721R, 721W, 740L, 740S, 743S, 746Q, 782A, 804G, 807G, 807H, 8141, 814M, 814R, 829L, and 829R, or combinations thereof, wherein the amino acid positions are relative to the reference sequence of SEQ ID NO: 994.

In some embodiments, the engineered DNA polymerase comprises one or more substitutions or substitution sets selected from H2G, H2V, H5A, H5G, H5L, H5V, H5W, G8R, M12V/L15V/G269R/K355F/G412A/E413K/Y492R/R704K, M12V/R252L/E413K/Y492R, L38G, L38R, L38S, L38V, G40R, G40S, E65G, E65H, E65S, S80G, S80M, E140M, R151K, E218Y, L226E, D230G, L242A, L242G, L242R, R258L/P347H, K279R, R284A, R284G, S289D, E298A, E298S, P300F, P300N, P300R, E304C, E304G, E304R, E304V, P309G, P311C, P311G, E329L, L331D, L331G, L331Y, R340T, D375A/L376I, D375E, D375L, E406G, R420S, R420T, R434K, E441R, H489R, S490R, S490V, T518M, T518R, E529V, Q543A, Q543L, Q543R, A560G, N592A, N592S, E635I, R639Q, K645R, L658Q, L658R, L666G, L666Q, L666R, L666V, H685F, R704V, K713V, E717T, K718R, E721D, E721M, E721R, E721W, P740L, P740S, N743S, V746Q, R782A, R804G, E807G, E807H, E814I, E814M, E814R, E829L, and E829R, or combinations thereof, wherein the amino acid positions are relative to the reference sequence of SEQ ID NO: 994.

In some embodiments, the engineered DNA polymerase comprises one or more substitutions or substitution sets at amino acid positions selected from 2, 5, 8, 12/15/269/355/412/413/492/704, 12/252/413/492, 32, 38, 40, 65, 98, 140, 151, 169, 202, 218, 226, 227, 230, 242, 258/347, 279, 284, 298, 300, 304, 309, 311, 329, 331, 340, 375, 375/376, 406, 420, 434, 440, 441, 481, 489, 490, 518, 529, 543, 560, 592, 635, 639, 645, 658, 666, 685, 692, 694, 704, 713, 717, 718, 721, 740, 746, 782, 804, 807, 814, and 829, or combinations thereof, wherein the amino acid positions are relative to the reference sequence of SEQ ID NO: 994.

In some embodiments, the engineered DNA polymerase comprises one or more substitutions or substitution sets selected from 2G, 2P, 2V, 5L, 5V, 8R, 12V/15V/269R/355F/412A/413K/492R/704K, 12V/252L/413K/492R, 32L, 38V, 40N, 40S, 65G, 65H, 65S, 98G, 98N, 140M, 151K, 169S, 202Q, 218G, 218Y, 226E, 227Y, 230G, 242A, 242G, 242R, 258L/347H, 279R, 284G, 298A, 298S, 300A, 300F, 300T, 304C, 304G, 304V, 309G, 311G, 329L, 329S, 331G, 331Q, 331V, 331Y, 340S, 340T, 375A, 375A/376I, 375E, 375L, 375M, 375R, 406M, 406Q, 420S, 420T, 434K, 440S, 440T, 441G, 481D, 481G, 489R, 490R, 490V, 518L, 518M, 529L, 529T, 529V, 543A, 543L, 543R, 560G, 560K, 592A, 592S, 635G, 635I, 635T, 639Q, 645F, 645L, 645M, 645N, 645R, 658E, 658Q, 658R, 658S, 666D, 666E, 666G, 666K, 666N, 666Q, 666R, 666S, 666V, 685F, 692S, 694S, 704Q, 704V, 713S, 713V, 717T, 717Y, 718S, 721D, 721L, 721M, 721R, 721V, 721W, 740L, 740S, 746G, 746Q, 746R, 782A, 782S, 804A, 804D, 807G, 814A, 814M, 814R, 829L, and 829R, or combinations thereof, wherein the amino acid positions are relative to the reference sequence of SEQ ID NO: 994.

In some embodiments, the engineered DNA polymerase comprises one or more substitutions or substitution sets selected from H2G, H2P, H2V, H5L, H5V, G8R, M12V/L15V/G269R/K355F/G412A/E413K/Y492R/R704K, M12V/R252L/E413K/Y492R, Y32L, L38V, G40N, G40S, E65G, E65H, E65S, E98G, E98N, E140M, R151K, H169S, P202Q, E218G, E218Y, L226E, K227Y, D230G, L242A, L242G, L242R, R258L/P347H, K279R, R284G, E298A, E298S, P300A, P300F, P300T, E304C, E304G, E304V, P309G, P311G, E329L, E329S, L331G, L331Q, L331V, L331Y, R340S, R340T, D375A, D375A/L376I, D375E, D375L, D375M, D375R, E406M, E406Q, R420S, R420T, R434K, E440S, E440T, E441G, E481D, E481G, H489R, S490R, S490V, T518L, T518M, E529L, E529T, E529V, Q543A, Q543L, Q543R, A560G, A560K, N592A, N592S, E635G, E635I, E635T, R639Q, K645F, K645L, K645M, K645N, K645R, L658E, L658Q, L658R, L658S, L666D, L666E, L666G, L666K, L666N, L666Q, L666R, L666S, L666V, H685F, G692S, P694S, R704Q, R704V, K713S, K713V, E717T, E717Y, K718S, E721D, E721L, E721M, E721R, E721V, E721W, P740L, P740S, V746G, V746Q, V746R, R782A, R782S, R804A, R804D, E807G, E814A, E814M, E814R, E829L, and E829R, or combinations thereof, wherein the amino acid positions are relative to the reference sequence of SEQ ID NO: 994.

In some embodiments, the engineered DNA polymerase comprises one or more substitutions or substitution sets at amino acid positions selected from 12/15/269/355/412/413/492/704, 12/252/413/492, 194, 194/252, 194/252/358/412/467, 194/252/358/412/467/658/696/743, 194/252/412/696, 194/252/467/658, 194/252/467/696/743, 194/252/467/704, 194/252/696/704, 194/252/704/743, 194/252/743, 194/358, 194/358/412, 194/358/467/658/696/704, 194/358/658, 194/412, 194/412/467, 194/412/658, 194/467, 194/467/658, 194/467/658/743, 194/467/704, 194/467/743, 194/658, 194/743, 242, 252/358/696/704, 252/412/658, 252/467/696, 258/347, 284, 304, 331, 434, 489, 543, 560, 592, 658, 666, 685, 717, 721, and 814, or combinations thereof, wherein the amino acid positions are relative to the reference sequence of SEQ ID NO: 994.

In some embodiments, the engineered DNA polymerase comprises one or more substitutions or substitution sets selected from 12V/15V/269R/355F/412A/413K/492R/704K, 12V/252L/413K/492R, 194V, 194V/252L, 194V/252L/358N/412A/467N, 194V/252L/358N/412A/467N/658V/696T/743T, 194V/252L/412A/696T, 194V/252L/467N/658V, 194V/252L/467N/696T/743T, 194V/252L/467N/704K, 194V/252L/696T/704K, 194V/252L/704K/743T, 194V/252L/743T, 194V/358N, 194V/358N/412A, 194V/358N/467N/658V/696T/704K, 194V/358N/658V, 194V/412A, 194V/412A/467N, 194V/412A/658V, 194V/467N, 194V/467N/658V, 194V/467N/658V/743T, 194V/467N/704K, 194V/467N/743T, 194V/658V, 194V/743T, 242G, 252L/358N/696T/704K, 252L/412A/658V, 252L/467N/696T, 258L/347H, 284A, 304G, 304V, 331G, 434K, 489R, 543A, 560G, 592A, 658E, 666R, 685F, 717T, 721M, 721R, and 814M, or combinations thereof, wherein the amino acid positions are relative to the reference sequence of SEQ ID NO: 994.

In some embodiments, the engineered DNA polymerase comprises one or more substitutions or substitution sets selected from M12V/L15V/G269R/K355F/G412A/E413K/Y492R/R704K, M12V/R252L/E413K/Y492R, A194V, A194V/R252L, A194V/R252L/R358N/G412A/A467N, A194V/R252L/R358N/G412A/A467N/L658V/E696T/N743T, A194V/R252L/G412A/E696T, A194V/R252L/A467N/L658V, A194V/R252L/A467N/E696T/N743T, A194V/R252L/A467N/R704K, A194V/R252L/E696T/R704K, A194V/R252L/R704K/N743T, A194V/R252L/N743T, A194V/R358N, A194V/R358N/G412A, A194V/R358N/A467N/L658V/E696T/R704K, A194V/R358N/L658V, A194V/G412A, A194V/G412A/A467N, A194V/G412A/L658V, A194V/A467N, A194V/A467N/L658V, A194V/A467N/L658V/N743T, A194V/A467N/R704K, A194V/A467N/N743T, A194V/L658V, A194V/N743T, L242G, R252L/R358N/E696T/R704K, R252L/G412A/L658V, R252L/A467N/E696T, R258L/P347H, R284A, E304G, E304V, L331G, R434K, H489R, Q543A, A560G, N592A, L658E, L666R, H685F, E717T, E721M, E721R, and 814M, or combinations thereof, wherein the amino acid positions are relative to the reference sequence of SEQ ID NO: 994.

The present invention further provides engineered DNA polymerases or functional fragments thereof, comprising a polypeptide sequence having at least 75% or more sequence identity to a reference sequence comprising residues 12 to 841 of SEQ ID NO: 1858, or to a reference sequence of SEQ ID NO: 1858, wherein the polypeptide sequence comprises one or more substitutions relative to the reference sequence comprising residues 12 to 841 of SEQ ID NO: 1858, or to the reference sequence of SEQ ID NO: 1858.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 1858 or to the reference sequence of SEQ ID NO: 1858.

In some embodiments, the engineered DNA polymerase comprises one or more substitutions or substitution sets at amino acid positions selected from 194/252/269/304/467, 194/252/304, 194/252/304/358/467/658, 194/252/304/467, 194/252/358/489, 194/252/467, 194/269, 194/269/304, 194/269/304/658/717, 194/269/717, 194/304/358/467/489, 194/467/721, 194/536/658, 194/721, 252, 252/269/304/743, 252/304/489/696/717, 252/304/658, 252/358/467/489/685, 252/685, 269, 269/304, 269/304/467, 269/304/658, 269/658, 269/721, 304, 304/358/467/592, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

In some embodiments, the engineered DNA polymerase comprises one or more substitutions or substitution sets selected from 194V/252L/269G/304G/467N, 194V/252L/304G, 194V/252L/304G/358N/467N/658E, 194V/252L/304G/467N, 194V/252L/358N/489R, 194V/252L/467N, 194V/269G, 194V/269G/304G, 194V/269G/304V/658V/717T, 194V/269G/717T, 194V/304V/358N/467N/489R, 194V/467N/721R, 194V/536G/658E, 194V/721R, 252L, 252L/269G/304V/743T, 252L/304G/489R/696T/717T, 252L/304G/658V, 252L/358N/467N/489R/685F, 252L/685F, 269G, 269G/304G, 269G/304G/658E, 269G/304V/467N, 269G/658V, 269G/721R, 304G, 304G/358N/467N/592A, 304G/467N/489R/717T, 304G/489R/658V/721R, 304G/685F, 304V, 304V/489R/658E, 358N/658V, 489R, 489R/696T/743T, 685F, 696T, 717T, and 721R, or combinations thereof, wherein the amino acid positions are relative to the reference sequence of SEQ ID NO: 1858.

In some embodiments, the engineered DNA polymerase comprises one or more substitutions or substitution sets selected from A194V/R252L/R269G/E304G/A467N, A194V/R252L/E304G, A194V/R252L/E304G/R358N/A467N/L658E, A194V/R252L/E304G/A467N, A194V/R252L/R358N/H489R, A194V/R252L/A467N, A194V/R269G, A194V/R269G/E304G, A194V/R269G/E304V/L658V/E717T, A194V/R269G/E717T, A194V/E304V/R358N/A467N/H489R, A194V/A467N/E721R, A194V/P536G/L658E, A194V/E721R, R252L, R252L/R269G/E304V/N743T, R252L/E304G/H489R/E696T/E717T, R252L/E304G/L658V, R252L/R358N/A467N/H489R/H685F, R252L/H685F, R269G, R269G/E304G, R269G/E304G/L658E, R269G/E304V/A467N, R269G/L658V, R269G/E721R, E304G, E304G/R358N/A467N/N592A, E304G/A467N/H489R/E717T, E304G/H489R/L658V/E721R, E304G/H685F, E304V, E304V/H489R/L658E, R358N/L658V, H489R, H489R/E696T/N743T, H685F, E696T, E717T, and E721R, or combinations thereof, wherein the amino acid positions are relative to the reference sequence of SEQ ID NO: 1858.

In some embodiments, the engineered DNA polymerase comprises one or more substitutions or substitution sets selected from 252, 252/304, 252/304/489/696/717, 252/304/658, 252/358/467/489/685, 252/685, 269, 269/304/467, 269/304/489/685, 269/467/489/658/685, 269/658, 269/721, 304, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

In some embodiments, the engineered DNA polymerase comprises one or more substitutions or substitution sets selected from 252L, 252L/304G/489R/696T/717T, 252L/304G/658V, 252L/304V, 252L/358N/467N/489R/685F, 252L/685F, 269G, 269G/304G/489R/685F, 269G/304V/467N, 269G/467N/489R/658V/685F, 269G/658V, 269G/721R, 304G/467N/489R/717T, 304G/489R/658V/721R, 304G/685F, 304V, 304V/489R/658E, 358N/658V, 489R, 489R/696T/743T, 685F, 696T, 717T, and 721R, or combinations thereof, wherein the amino acid positions are relative to the reference sequence of SEQ ID NO: 1858.

In some embodiments, the engineered DNA polymerase comprises one or more substitutions or substitution sets selected from R252L, R252L/E304G/H489R/E696T/E717T, R252L/E304G/L658V, R252L/E304V, R252L/R358N/A467N/H489R/H685F, R252L/H685F, R269G, R269G/E304G/H489R/H685F, R269G/E304V/A467N, R269G/A467N/H489R/L658V/H685F, R269G/L658V, R269G/E721R, E304G/A467N/H489R/E717T, E304G/H489R/L658V/E721R, E304G/H685F, E304V, E304V/H489R/L658E, R358N/L658V, H489R, H489R/E696T/N743T, H685F, E696T, E717T, and E721R, or combinations thereof, wherein the amino acid positions are relative to the reference sequence of SEQ ID NO: 1858.

In some embodiments, the engineered DNA polymerase comprises one or more substitutions or substitution sets at amino acid positions selected from 252/685, 269, 269/658, 304/467/489/717, 304/489/658/721, 304/685, 358/658, 489, 685, 696, 717, and 721, or combinations thereof, wherein the amino acid positions are relative to the reference sequence of SEQ ID NO: 1858.

In some embodiments, the engineered DNA polymerase comprises one or more substitutions or substitution sets selected from 252L/685F, 269G, 269G/658V, 304G/467N/489R/717T, 304G/489R/658V/721R, 304G/685F, 358N/658V, 489R, 685F, 696T, 717T, and 721R, or combinations thereof, wherein the amino acid positions are relative to the reference sequence of SEQ ID NO: 1858.

In some embodiments, the engineered DNA polymerase comprises one or more substitutions or substitution sets selected from R252L/H685F, R269G, R269G/L658V, E304G/A467N/H489R/E717T, E304G/H489R/L658V/E721R, E304G/H685F, R358N/L658V, H489R, H685F, E696T, E717T, and E721R, or combinations thereof, wherein the amino acid positions are relative to the reference sequence of SEQ ID NO: 1858.

The present invention also provides engineered DNA polymerases or functional fragments thereof, comprising a polypeptide sequence having at least 75% or more sequence identity to a reference sequence comprising residues 12 to 841 of SEQ ID NO: 2176, or to a reference sequence of SEQ ID NO: 2176, wherein the polypeptide sequence comprises one or more substitutions relative to the reference sequence comprising residues 12 to 841 of SEQ ID NO: 2176, or to the reference sequence of SEQ ID NO: 2176.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2176 or to the reference sequence of SEQ ID NO: 2176.

In some embodiments, the engineered DNA polymerase comprises one or more substitutions or substitution sets at amino acid positions selected from 40, 42, 45, 45/65/243/268, 45/109/340/605/814, 45/221/243/340/423, 45/243/312/331/451, 45/243/312/331/557, 45/243/312/340/423/543/814, 45/243/814, 45/268/312/605/748/749/814, 45/268/489/557/748/749, 45/312/340/605, 45/340/423/814, 45/340/557, 45/423, 45/431, 45/451, 45/475/557/748/749, 45/489/557/749, 45/621, 45/699/748/749, 57, 61, 64/293, 65, 65/233/543/621, 65/489/543, 81, 94, 102, 109, 109/340/489/510/659, 118, 124, 145, 151, 177, 186, 194, 194/269/489, 194/358, 210, 211, 221, 223, 227, 227/443/592/617, 227/443/756, 233, 242, 243, 243/268, 252, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 289, 292, 292/430, 298, 300, 301, 301/538/546/621/814, 302, 302/340/489/494/510/748/814, 302/340/494, 302/494, 302/494/525, 302/494/543/659/814, 304/489, 312, 312/331/605/814, 312/340/494, 316, 323, 329, 331, 331/605, 340, 340/489, 340/489/494/543/659/814, 340/489/699, 350, 352, 352/451/489/543/557/721, 355, 355/430, 358/467, 371, 397, 406, 407, 420, 423, 431, 441, 451, 463, 466, 475, 481, 488, 489, 489/494, 489/510, 489/621/814, 489/658, 492, 494, 501, 510, 510/748, 519, 521, 530, 533, 534, 536, 538, 543, 543/699/814, 553, 557, 557/605/699/721, 560, 597, 605, 621, 639, 644, 659, 660, 666, 670, 685, 686, 692, 694, 696, 699, 704, 717, 721, 722, 726, 732, 738, 743, 748, 748/749, 749, 756, 802, 804, 814, 827, 829, and 834, or combinations thereof, wherein the amino acid positions are relative to the reference sequence of SEQ ID NO: 2176.

In some embodiments, the engineered DNA polymerase comprises one or more substitutions or substitution sets selected from 40C, 40S, 42V, 45G, 45L, 45L/621R, 45M, 45S, 45S/65N/243N/268Q, 45S/109H/340T/605K/814T, 45S/221D/243N/340T/423E, 45S/243N/312Q/331A/451G, 45S/243N/312Q/331A/557R, 45S/243N/312Q/340T/423E/543A/814T, 45S/243N/814T, 45S/268G/312Q/605K/748G/749K/814T, 45S/268Q/489R/557R/748G/749K, 45S/312Q/340T/605K, 45S/340T/423E/814T, 45S/340T/557R, 45S/423E, 45S/431T, 45S/451G, 45S/475H/557R/748G/749K, 45S/489R/557R/749K, 45S/699Q/748G/749K, 45V, 571, 57R, 61G, 64E/293H, 65C, 65K/489R/543A, 65N, 65N/233S/543V/621R, 65S, 81W, 94K, 94L, 94M, 94R, 102D, 102E, 102G, 102T, 109C, 109H, 109H/340T/489R/510R/659G, 109T, 109Y, 118L, 118T, 118V, 124A, 145F, 151S, 177F, 186M, 194G, 194V/269G/489R, 194V/358N, 210G, 210R, 210S, 211L, 211R, 221C, 221D, 221T, 223D, 227E, 227E/443D/592S/617V, 227E/443D/756R, 233E, 233L, 233R, 233S, 242Q, 242V, 243N, 243N/268G, 243S, 252L, 268E, 268G, 268G/423E/814T, 268G/431T/605K/749K, 268K, 268Q, 268Q/312Q/331A/340T/699Q, 268Q/312Q/331A/423E/451G/557R/605K, 268Q/340T/352Q/423E/451G, 268Q/423E, 268Q/423E/431T/543A/557R/605K/814T, 268Q/423E/814T, 268V, 269G, 269G/658E, 2691, 269L, 269M, 269S, 269V, 272L, 272V, 280E, 280G, 280K, 283S, 284E, 284G, 289E, 292E, 292E/430H, 292G, 292M, 292R, 292W, 298G, 298I, 300E, 300G, 300L, 300M, 300S, 300T, 301C, 301E, 301G, 301G/538M/546A/621R/814T, 302L/340T/489R/494S/510R/748G/814T, 302V, 302V/340T/494V, 302V/494V, 302V/494V/525R, 302V/494V/543V/659G/814T, 304G/489R, 312D, 312Q, 312Q/331A/605K/814T, 312Q/340T/494V, 316T, 323C, 329M, 329S, 329T, 329V, 331A, 331K, 331M, 331S, 331S/605K, 331V, 340A, 340K, 340L, 340T, 340T/489R, 340T/489R/494V/543A/659G/814T, 340T/489R/699Q, 340V, 350C, 350G, 350S, 352Q, 352Q/451G/489R/543V/557R/721R, 355C, 355L/430R, 355T, 355V, 358N/467N, 371C, 371S, 397V, 406G, 406L, 406W, 407L, 407V, 420H, 423E, 423R, 431T, 431V, 441L, 441M, 451G, 463L, 463W, 466H, 466K, 466M, 475C, 475H, 481A, 481S, 488A, 488R, 488S, 489R, 489R/494V, 489R/510R, 489R/621R/814T, 489R/658E, 492A, 494H, 494R, 494V, 494Y, 501V, 510D, 510L, 510M, 510R, 510R/748G, 519F, 519L, 519V, 521W, 530D, 530E, 530G, 530T, 530V, 530W, 533P, 533T, 534A, 534G, 534R, 534W, 536D, 536G, 536T, 538M, 543A, 543L, 543V/699S/814T, 553Q, 553V, 557L, 557M, 557R, 557R/605K/699Q/721R, 557S, 560M, 560S, 560V, 597G, 597N, 597S, 597V, 605K, 621D, 621E, 621G, 621K, 621Q, 621R, 621T, 639K, 644R, 659A, 659C, 659T, 660G, 660V, 666S, 666W, 670M, 685G, 685V, 686G, 692R, 694T, 696Q, 699G, 699Q, 699S, 704R, 717D, 721A, 721R, 722V, 722W, 726M, 732M, 738P, 743G, 748G, 748G/749K, 749G, 749K, 749S, 749T, 7561, 756L, 802C, 802R, 804A, 804G, 814G, 814K, 814M, 814Q, 814R, 827R, 829R, and 834L, or combinations thereof, wherein the amino acid positions are relative to the reference sequence of SEQ ID NO: 2176.

In some embodiments, the engineered DNA polymerase comprises one or more substitutions or substitution sets selected from G40C, G40S, T42V, R45G, R45L, R45L/S621R, R45M, R45S, R45S/E65N/A243N/R268Q, R45S/E109H/R340T/R605K/E814T, R45S/S221D/A243N/R340T/A423E, R45S/A243N/E312Q/L331A/A451G, R45S/A243N/E312Q/L331A/P557R, R45S/A243N/E312Q/R340T/A423E/Q543A/E814T, R45S/A243N/E814T, R45S/R268G/E312Q/R605K/S748G/V749K/E814T, R45S/R268Q/H489R/P557R/S748G/V749K, R45S/E312Q/R340T/R605K, R45S/R340T/A423E/E814T, R45S/R340T/P557R, R45S/A423E, R45S/G431T, R45S/A451G, R45S/E475H/P557R/S748G/V749K, R45S/H489R/P557R/V749K, R45S/A699Q/S748G/V749K, R45V, K57I, K57R, K61G, K64E/E293H, E65C, E65K/H489R/Q543A, E65N, E65N/K233S/Q543V/S621R, E65S, F81W, A94K, A94L, A94M, A94R, R102D, R102E, R102G, R102T, E109C, E109H, E109H/R340T/H489R/P510R/S659G, E109T, E109Y, R118L, R118T, R118V, F124A, Q145F, R151S, W177F, Q186M, A194G, A194V/R269G/H489R, A194V/R358N, K210G, K210R, K210S, T211L, T211R, S221C, S221D, S221T, E223D, K227E, K227E/E443D/N592S/A617V, K227E/E443D/M756R, K233E, K233L, K233R, K233S, L242Q, L242V, A243N, A243N/R268G, A243S, R252L, R268E, R268G, R268G/A423E/E814T, R268G/G431T/R605K/V749K, R268K, R268Q, R268Q/E312Q/L331A/R340T/A699Q, R268Q/E312Q/L331A/A423E/A451G/P557R/R605K, R268Q/R340T/R352Q/A423E/A451G, R268Q/A423E, R268Q/A423E/G431T/Q543A/P557R/R605K/E814T, R268Q/A423E/E814T, R268V, R269G, R269G/L658E, R269I, R269L, R269M, R269S, R269V, E272L, E272V, A280E, A280G, A280K, E283S, R284E, R284G, S289E, H292E, H292E/Q430H, H292G, H292M, H292R, H292W, E298G, E298I, P300E, P300G, P300L, P300M, P300S, P300T, L301C, L301E, L301G, L301G/V538M/E546A/S621R/E814T, P302L/R340T/H489R/N494S/P510R/S748G/E814T, P302V, P302V/R340T/N494V, P302V/N494V, P302V/N494V/A525R, P302V/N494V/Q543V/S659G/E814T, E304G/H489R, E312D, E312Q, E312Q/L331A/R605K/E814T, E312Q/R340T/N494V, L316T, P323C, E329M, E329S, E329T, E329V, L331A, L331K, L331M, L331S, L331S/R605K, L331V, R340A, R340K, R340L, R340T, R340T/H489R, R340T/H489R/N494V/Q543A/S659G/E814T, R340T/H489R/A699Q, R340V, A350C, A350G, A350S, R352Q, R352Q/A451G/H489R/Q543V/P557R/E721R, F355C, F355L/Q430R, F355T, F355V, R358N/A467N, R371C, R371S, E397V, E406G, E406L, E406W, W407L, W407V, R420H, A423E, A423R, G431T, G431V, E441L, E441M, A451G, A463L, A463W, E466H, E466K, E466M, E475C, E475H, E481A, E481S, G488A, G488R, G488S, H489R, H489R/N494V, H489R/P510R, H489R/S621R/E814T, H489R/L658E, R492A, N494H, N494R, N494V, N494Y, R501V, P510D, P510L, P510M, P510R, P510R/S748G, G519F, G519L, G519V, R521W, A530D, A530E, A530G, A530T, A530V, A530W, E533P, E533T, V534A, V534G, V534R, V534W, P536D, P536G, P536T, V538M, Q543A, Q543L, Q543V/A699S/E814T, T553Q, T553V, P557L, P557M, P557R, P557R/R605K/A699Q/E721R, P557S, A560M, A560S, A560V, T597G, T597N, T597S, T597V, R605K, S621D, S621E, S621G, S621K, S621Q, S621R, S621T, R639K, G644R, S659A, S659C, S659T, A660G, A660V, L666S, L666W, A670M, F685G, F685V, R686G, G692R, P694T, E696Q, A699G, A699Q, A699S, K704R, E717D, E721A, E721R, E722V, E722W, R726M, L732M, Y738P, N743G, S748G, S748G/V749K, V749G, V749K, V749S, V749T, M756I, M756L, K802C, K802R, R804A, R804G, E814G, E814K, E814M, E814Q, E814R, E827R, E829R, and E834L, or combinations thereof, wherein the amino acid positions are relative to the reference sequence of SEQ ID NO: 2176.

In some embodiments, the engineered DNA polymerase comprises one or more substitutions or substitution sets at amino acid positions selected from 45, 45/65/243/268, 45/109/243/423/543/605/699/748/749/814, 45/109/340/605/814, 45/221/243/340/423, 45/243/312/331/451, 45/243/312/331/557, 45/243/312/340/423/543/814, 45/243/312/605, 45/243/814, 45/268/312/605/748/749/814, 45/268/489/543/557/659/721, 45/268/489/557/748/749, 45/312, 45/312/340/605, 45/340/423/814, 45/340/475/557/814, 45/340/557, 45/423, 45/431, 45/451, 45/475/557/748/749, 45/489/557/749, 45/621, 45/699/748/749, 64/293, 65, 65/489/543, 81, 94, 102, 109, 109/340/489/510/659, 109/423, 118, 120, 151, 162, 194, 211, 221, 233, 243, 243/268, 245, 252, 258, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 292, 298, 300, 301, 301/538/546/621/814, 302/340/489/494/510/748/814, 302/340/489/494/525, 302/340/494, 302/494, 302/494/525, 304/489, 312, 312/331/605/814, 312/423/431/451/489, 312/494/525/543/659/748/814, 323, 329, 331, 331/605, 340, 340/489, 340/489/494/543/659/814, 340/489/699, 350, 352, 352/451/489/543/557/721, 355, 355/430, 358/467, 406, 407, 423, 423/463/605, 431, 451, 463, 466, 467, 475, 476, 481, 489, 489/494, 489/494/659, 489/510, 489/658, 494, 501, 510, 510/748, 519, 530, 533, 534, 538, 543, 543/699/814, 557, 557/605/699/721, 560, 597, 605, 621, 659, 666, 694, 696, 699, 721, 722, 748, 748/749, 749, 756, 802, and 814, or combinations thereof, wherein the amino acid positions are relative to the reference sequence of SEQ ID NO: 2176.

In some embodiments, the engineered DNA polymerase comprises one or more substitutions or substitution sets selected from 45G, 45L, 45L/621R, 45S, 45S/65N/243N/268Q, 45S/109H/243N/423E/543V/605K/699Q/748G/749K/814T, 45S/109H/340T/605K/814T, 45S/221D/243N/340T/423E, 45S/243N/312Q/331A/451G, 45S/243N/312Q/331A/557R, 45S/243N/312Q/340T/423E/543A/814T, 45S/243N/312Q/605K, 45S/243N/814T, 45S/268G/312Q/605K/748G/749K/814T, 45S/268Q/489R/543V/557R/659T/721R, 45S/268Q/489R/557R/748G/749K, 45S/312Q, 45S/312Q/340T/605K, 45S/340T/423E/814T, 45S/340T/475H/557R/814T, 45S/340T/557R, 45S/423E, 45S/431T, 45S/451G, 45S/475H/557R/748G/749K, 45S/489R/557R/749K, 45S/699Q/748G/749K, 45V, 64H/293D, 65C, 65K/489R/543A, 65N, 65S, 81W, 94K, 102P, 109H, 109H/340T/489R/510R/659G, 109H/423E, 118L, 118V, 120D, 151G, 162T, 194G, 211R, 221D, 233R, 243N, 243N/268G, 243S, 245T, 252L, 258L, 268E, 268G, 268G/423E/814T, 268G/431T/605K/749K, 268Q, 268Q/312Q/331A/340T/699Q, 268Q/312Q/331A/423E/451G/557R/605K, 268Q/340T/352Q/423E/451G, 268Q/423E, 268Q/423E/431T/543A/557R/605K/814T, 268Q/423E/814T, 269G, 269G/658E, 2691, 272V, 280G, 283A, 283L, 283S, 284G, 292R, 298D, 298I, 300E, 300G, 300L, 300M, 300S, 300T, 301C, 301E, 301G, 301G/538M/546A/621R/814T, 302L/340T/489R/494S/510R/748G/814T, 302L/340T/489R/494V/525R, 302V/340T/494V, 302V/494V, 302V/494V/525R, 304G/489R, 312Q, 312Q/331A/605K/814T, 312Q/423E/431T/451G/489R, 312Q/494V/525R/543V/659G/748G/814T, 323W, 329M, 329S, 331A, 331S/605K, 331V, 340L, 340T, 340T/489R, 340T/489R/494V/543A/659G/814T, 340T/489R/699Q, 350C, 350G, 350Y, 352Q, 352Q/451G/489R/543V/557R/721R, 355C, 355L/430R, 355T, 358N/467N, 406G, 406L, 407V, 423E, 423E/463W/605K, 431T, 431V, 451G, 463W, 466H, 466M, 467N, 475C, 475H, 475L, 476V, 481S, 489R, 489R/494V, 489R/494V/659G, 489R/510R, 489R/658E, 494Y, 501V, 510M, 510R, 510R/748G, 519F, 519L, 530D, 530E, 530T, 530W, 533T, 534A, 534G, 538M, 543L, 543V, 543V/699S/814T, 557R, 557R/605K/699Q/721R, 560S, 597G, 597N, 597S, 605K, 621E, 621K, 621R, 659T, 666E, 666S, 666W, 694T, 696Q, 699G, 699Q, 699S, 721A, 721R, 722V, 748G, 748G/749K, 749G, 749K, 749S, 756L, 802C, 802R, 814K, and 814T, or combinations thereof, wherein the amino acid positions are relative to the reference sequence of SEQ ID NO: 2176.

In some embodiments, the engineered DNA polymerase comprises one or more substitutions or substitution sets selected from R45G, R45L, R45L/S621R, R45S, R45S/E65N/A243N/R268Q, R45S/E109H/A243N/A423E/Q543V/R605K/A699Q/S748G/V749K/E814T, R45S/E109H/R340T/R605K/E814T, R45S/S221D/A243N/R340T/A423E, R45S/A243N/E312Q/L331A/A451G, R45S/A243N/E312Q/L331A/P557R, R45S/A243N/E312Q/R340T/A423E/Q543A/E814T, R45S/A243N/E312Q/R605K, R45S/A243N/E814T, R45S/R268G/E312Q/R605K/S748G/V749K/E814T, R45S/R268Q/H489R/Q543V/P557R/S659T/E721R, R45S/R268Q/H489R/P557R/S748G/V749K, R45S/E312Q, R45S/E312Q/R340T/R605K, R45S/R340T/A423E/E814T, R45S/R340T/E475H/P557R/E814T, R45S/R340T/P557R, R45S/A423E, R45S/G431T, R45S/A451G, R45S/E475H/P557R/S748G/V749K, R45S/H489R/P557R/V749K, R45S/A699Q/S748G/V749K, R45V, K64H/E293D, E65C, E65K/H489R/Q543A, E65N, E65S, F81W, A94K, R102P, E109H, E109H/R340T/H489R/P510R/S659G, E109H/A423E, R118L, R118V, E120D, R151G, S162T, A194G, T211R, S221D, K233R, A243N, A243N/R268G, A243S, L245T, R252L, R258L, R268E, R268G, R268G/A423E/E814T, R268G/G431T/R605K/V749K, R268Q, R268Q/E312Q/L331A/R340T/A699Q, R268Q/E312Q/L331A/A423E/A451G/P557R/R605K, R268Q/R340T/R352Q/A423E/A451G, R268Q/A423E, R268Q/A423E/G431T/Q543A/P557R/R605K/E814T, R268Q/A423E/E814T, R269G, R269G/L658E, R269I, E272V, A280G, E283A, E283L, E283S, R284G, H292R, E298D, E298I, P300E, P300G, P300L, P300M, P300S, P300T, L301C, L301E, L301G, L301G/V538M/E546A/S621R/E814T, P302L/R340T/H489R/N494S/P510R/S748G/E814T, P302L/R340T/H489R/N494V/A525R, P302V/R340T/N494V, P302V/N494V, P302V/N494V/A525R, E304G/H489R, E312Q, E312Q/L331A/R605K/E814T, E312Q/A423E/G431T/A451G/H489R, E312Q/N494V/A525R/Q543V/S659G/S748G/E814T, P323W, E329M, E329S, L331A, L331S/R605K, L331V, R340L, R340T, R340T/H489R, R340T/H489R/N494V/Q543A/S659G/E814T, R340T/H489R/A699Q, A350C, A350G, A350Y, R352Q, R352Q/A451G/H489R/Q543V/P557R/E721R, F355C, F355L/Q430R, F355T, R358N/A467N, E406G, E406L, W407V, A423E, A423E/A463W/R605K, G431T, G431V, A451G, A463W, E466H, E466M, A467N, E475C, E475H, E475L, M476V, E481S, H489R, H489R/N494V, H489R/N494V/S659G, H489R/P510R, H489R/L658E, N494Y, R501V, P510M, P510R, P510R/S748G, G519F, G519L, A530D, A530E, A530T, A530W, E533T, V534A, V534G, V538M, Q543L, Q543V, Q543V/A699S/E814T, P557R, P557R/R605K/A699Q/E721R, A560S, T597G, T597N, T597S, R605K, S621E, S621K, S621R, S659T, L666E, L666S, L666W, P694T, E696Q, A699G, A699Q, A699S, E721A, E721R, E722V, S748G, S748G/V749K, V749G, V749K, V749S, M756L, K802C, K802R, E814K, and E814T, or combinations thereof, wherein the amino acid positions are relative to the reference sequence of SEQ ID NO: 2176.

In some embodiments, the engineered DNA polymerase comprises one or more substitutions or substitution sets at amino acid positions selected from 45, 45/65/243/268, 45/221/243/340/423, 45/268/312/605/748/749/814, 45/268/489/557/748/749, 45/451, 45/475/557/748/749, 45/489/557/749, 45/621, 45/699/748/749, 65, 65/489/543, 81, 94, 102, 109, 109/340/489/510/659, 221, 227/443/756, 252, 268, 268/312/331/340/699, 268/312/331/423/451/557/605, 268/423/431/543/557/605/814, 269, 283, 300, 301, 301/538/546/621/814, 302/340/489/494/510/748/814, 302/340/489/494/525, 302/494/525, 304/489, 312, 312/494/525/543/659/748/814, 329, 331, 340, 340/489/699, 352, 358/467, 406, 431, 451, 463, 466, 475, 481, 489, 489/510, 489/658, 510, 510/748, 530, 543, 543/699/814, 557, 557/605/699/721, 560, 605, 659, 666, 696, 699, 721, 722, 748, 748/749, 749, 802, and 814, or combinations thereof, wherein the amino acid positions are relative to the reference sequence of SEQ ID NO: 2176.

In some embodiments, the engineered DNA polymerase comprises one or more substitutions or substitution sets selected from 45L/621R, 45S, 45S/65N/243N/268Q, 45S/221D/243N/340T/423E, 45S/268G/312Q/605K/748G/749K/814T, 45S/268Q/489R/557R/748G/749K, 45S/451G, 45S/475H/557R/748G/749K, 45S/489R/557R/749K, 45S/699Q/748G/749K, 65K/489R/543A, 65N, 81W, 94K, 102P, 109H, 109H/340T/489R/510R/659G, 221D, 227E/443D/756R, 252L, 268G, 268Q, 268Q/312Q/331A/340T/699Q, 268Q/312Q/331A/423E/451G/557R/605K, 268Q/423E/431T/543A/557R/605K/814T, 269G, 283L, 300M, 300S, 300T, 301G, 301G/538M/546A/621R/814T, 302L/340T/489R/494S/510R/748G/814T, 302L/340T/489R/494V/525R, 302V/494V/525R, 304G/489R, 312Q, 312Q/494V/525R/543V/659G/748G/814T, 329S, 331A, 340T, 340T/489R/699Q, 352Q, 358N/467N, 406L, 431T, 451G, 463W, 466M, 475C, 475H, 481S, 489R, 489R/510R, 489R/658E, 510R, 510R/748G, 530D, 530E, 530T, 543V, 543V/699S/814T, 557R, 557R/605K/699Q/721R, 560S, 605K, 659T, 666E, 666S, 666W, 696Q, 699Q, 721R, 722V, 748G, 748G/749K, 749K, 802C, and 814T, or combinations thereof, wherein the amino acid positions are relative to the reference sequence of SEQ ID NO: 2176.

In some embodiments, the engineered DNA polymerase comprises one or more substitutions or substitution sets selected from R45L/S621R, R45S, R45S/E65N/A243N/R268Q, R45S/S221D/A243N/R340T/A423E, R45S/R268G/E312Q/R605K/S748G/V749K/E814T, R45S/R268Q/H489R/P557R/S748G/V749K, R45S/A451G, R45S/E475H/P557R/S748G/V749K, R45S/H489R/P557R/V749K, R45S/A699Q/S748G/V749K, E65K/H489R/Q543A, E65N, F81W, A94K, R102P, E109H, E109H/R340T/H489R/P510R/S659G, S221D, K227E/E443D/M756R, R252L, R268G, R268Q, R268Q/E312Q/L331A/R340T/A699Q, R268Q/E312Q/L331A/A423E/A451G/P557R/R605K, R268Q/A423E/G431T/Q543A/P557R/R605K/E814T, R269G, E283L, P300M, P300S, P300T, L301G, L301G/V538M/E546A/S621R/E814T, P302L/R340T/H489R/N494S/P510R/S748G/E814T, P302L/R340T/H489R/N494V/A525R, P302V/N494V/A525R, E304G/H489R, E312Q, E312Q/N494V/A525R/Q543V/S659G/S748G/E814T, E329S, L331A, R340T, R340T/H489R/A699Q, R352Q, R358N/A467N, E406L, G431T, A451G, A463W, E466M, E475C, E475H, E481S, H489R, H489R/P510R, H489R/L658E, P510R, P510R/S748G, A530D, A530E, A530T, Q543V, Q543V/A699S/E814T, P557R, P557R/R605K/A699Q/E721R, A560S, R605K, S659T, L666E, L666S, L666W, E696Q, A699Q, E721R, E722V, S748G, S748G/V749K, V749K, K802C, and E814T, or combinations thereof, wherein the amino acid positions are relative to the reference sequence of SEQ ID NO: 2176.

In some embodiments, the engineered DNA polymerase comprises one or more substitutions or substitution sets at amino acid positions selected from 45/65/243/268, 45/268/312/605/748/749/814, 45/268/489/557/748/749, 45/451, 45/475/557/748/749, 45/489/557/749, 45/621, 45/699/748/749, 57, 65, 65/489/543, 81, 94, 109, 221, 243, 268, 268/312/331/340/699, 268/312/331/423/451/557/605, 268/423/431/543/557/605/814, 269, 300, 301, 302/340/489/494/510/748/814, 302/494/525, 304/489, 312, 331, 331/605, 340, 340/489/699, 352, 358/467, 406, 423, 431, 451, 463, 466, 475, 481, 489, 489/510, 489/658, 494, 510, 510/748, 530, 543, 543/699/814, 557, 557/605/699/721, 560, 597, 605, 659, 666, 696, 699, 721, 748, 748/749, 749, 802, and 814, or combinations thereof, wherein the amino acid positions are relative to the reference sequence of SEQ ID NO: 2176.

In some embodiments, the engineered DNA polymerase comprises one or more substitutions or substitution sets selected from 45L/621R, 45S/65N/243N/268Q, 45S/268G/312Q/605K/748G/749K/814T, 45S/268Q/489R/557R/748G/749K, 45S/451G, 45S/475H/557R/748G/749K, 45S/489R/557R/749K, 45S/699Q/748G/749K, 57R, 65K/489R/543A, 65N, 81W, 94K, 109H, 221D, 243N, 268G, 268Q, 268Q/312Q/331A/340T/699Q, 268Q/312Q/331A/423E/451G/557R/605K, 268Q/423E/431T/543A/557R/605K/814T, 269G, 300S, 300T, 301G, 302L/340T/489R/494S/510R/748G/814T, 302V/494V/525R, 304G/489R, 312Q, 331A, 331S/605K, 340L, 340T, 340T/489R/699Q, 352Q, 358N/467N, 406L, 423E, 431T, 451G, 463W, 466M, 475C, 475H, 481S, 489R, 489R/510R, 489R/658E, 494S, 510R, 510R/748G, 530D, 530E, 530T, 543V, 543V/699S/814T, 557R, 557R/605K/699Q/721R, 560S, 597G, 597N, 605K, 659T, 666E, 666S, 666W, 696Q, 699Q, 721A, 721R, 748G, 748G/749K, 749K, 802C, 802R, and 814, or combinations thereof, wherein the amino acid positions are relative to the reference sequence of SEQ ID NO: 2176.

In some embodiments, the engineered DNA polymerase comprises one or more substitutions or substitution sets selected from R45L/S621R, R45S/E65N/A243N/R268Q, R45S/R268G/E312Q/R605K/S748G/V749K/E814T, R45S/R268Q/H489R/P557R/S748G/V749K, R45S/A451G, R45S/E475H/P557R/S748G/V749K, R45S/H489R/P557R/V749K, R45S/A699Q/S748G/V749K, K57R, E65K/H489R/Q543A, E65N, F81W, A94K, E109H, S221D, A243N, R268G, R268Q, R268Q/E312Q/L331A/R340T/A699Q, R268Q/E312Q/L331A/A423E/A451G/P557R/R605K, R268Q/A423E/G431T/Q543A/P557R/R605K/E814T, R269G, P300S, P300T, L301G, P302L/R340T/H489R/N494S/P510R/S748G/E814T, P302V/N494V/A525R, E304G/H489R, E312Q, L331A, L331S/R605K, R340L, R340T, R340T/H489R/A699Q, R352Q, R358N/A467N, E406L, A423E, G431T, A451G, A463W, E466M, E475C, E475H, E481S, H489R, H489R/P510R, H489R/L658E, N494S, P510R, P510R/S748G, A530D, A530E, A530T, Q543V, Q543V/A699S/E814T, P557R, P557R/R605K/A699Q/E721R, A560S, T597G, T597N, R605K, S659T, L666E, L666S, L666W, E696Q, A699Q, E721A, E721R, S748G, S748G/V749K, V749K, K802C, K802R, and 814T, or combinations thereof, wherein the amino acid positions are relative to the reference sequence of SEQ ID NO: 2176.

The present invention also provides engineered DNA polymerases or functional fragments thereof, comprising a polypeptide sequence having at least 75% or more sequence identity to a reference sequence comprising residues 12 to 841 of SEQ ID NO: 2310, or to a reference sequence of SEQ ID NO: 2310, wherein the polypeptide sequence comprises one or more substitutions relative to the reference sequence comprising residues 12 to 841 of SEQ ID NO: 2310, or to the reference sequence of SEQ ID NO: 2310.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2310 or to the reference sequence of SEQ ID NO: 2310.

In some embodiments, the engineered DNA polymerase comprises one or more substitutions or substitution sets at amino acid positions selected from 18/268/302/312/605/659/699, 45/227/268/423/489/510/605, 45/227/302/423/510/605/621/699, 45/227/312/423/525/605/699, 45/268/302/423/494/621/699, 45/268/443/525/696, 45/331/489, 45/331/621, 45/340/423, 45/494/525/621/696/699, 45/605, 45/696/699, 65/685, 227, 227/268/340/451, 227/268/423/621, 227/302, 227/312/489/494/621, 227/331, 227/331/423/605, 227/331/605/659, 227/340/423/605, 227/340/621, 227/423, 227/423/451, 227/451/525/621/659/699/721, 227/525, 227/543/659/699, 227/605/721, 227/699, 268/302/340/358/605/699, 268/302/423/605, 268/340, 268/340/423/443/525/621/659, 268/340/423/621, 268/340/525/605, 268/423, 268/423/478/489/525/605/699, 268/423/489/494/605/659, 268/423/510/605, 268/423/605/699, 268/423/699, 268/467, 268/467/489, 268/467/489/510/699, 268/467/489/699, 268/467/489/699/814, 268/467/510/699/814, 268/489/494/605/699, 268/489/699, 268/510, 268/510/699/814, 268/525, 268/525/605, 268/543, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/451, 331/525/605/699, 331/605/699, 340/451/494/525, 358/467/489/699, 358/467/699, 358/467/699/814, 423/443/451/494/510, 423/451/605/659/699, 423/489/494/529/605/621/696/699, 423/494, 423/525, 423/605/621, 451/510, 467, 467/489, 467/489/510/543/699/814, 467/489/699, 467/489/699/814, 467/489/814, 467/510/699, 467/510/699/814, 467/510/814, 467/699, 484, 489, 489/494/605/721, 489/510/525/605/699, 489/699, 489/699/814, 494/605/699, 494/659, 494/699, 498, 503, 510, 510/605/659, 510/699, 510/699/814, 524, 526, 527, 531, 540, 542, 543/699, 543/699/814, 547, 569, 577/614, 605, 605/621/659/696/699, 605/621/696/699, 621, 659/699, 673, 684, 685, 685/714, 699, 699/814, 702, and 716, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/227E/312Q/423E/525R/605K/699Q, 45L/268Q/302L/423E/494S/621R/699S, 45L/268Q/443D/525R/696Q, 45L/331A/489R, 45L/331A/621R, 45L/340T/423E, 45L/494V/525R/621R/696Q/699Q, 45L/605K, 45L/696Q/699Q, 65K/685R, 227E, 227E/268G/423E/621R, 227E/268Q/340T/451G, 227E/302L, 227E/312Q/489R/494S/621R, 227E/331A/423E/605K, 227E/331S, 227E/331S/605K/659G, 227E/340T/423E/605K, 227E/340T/621R, 227E/423E, 227E/423E/451G, 227E/451G/525R/621R/659G/699Q/721R, 227E/525R, 227E/543V/659G/699S, 227E/605K/721R, 227E/699S, 268G/302L/423E/605K, 268G/340T, 268G/423E/478H/489R/525R/605K/699Q, 268G/423E/510R/605K, 268G/423E/699Q, 268G/621R, 268Q/302L/340T/358N/605K/699Q, 268Q/340T/423E/443D/525R/621R/659G, 268Q/340T/423E/621R, 268Q/340T/525R/605K, 268Q/423E, 268Q/423E/489R/494S/605K/659G, 268Q/423E/605K/699S, 268Q/467N, 268Q/467N/489R, 268Q/467N/489R/510R/699Q, 268Q/467N/489R/699Q, 268Q/467N/489R/699S/814T, 268Q/467N/510R/699S/814T, 268Q/489R/494S/605K/699Q, 268Q/489R/699Q, 268Q/510R, 268Q/510R/699Q/814T, 268Q/525R, 268Q/525R/605K, 268Q/543V, 268Q/543V/699Q, 268Q/605K/621R/721R, 268Q/699Q, 268Q/699S, 302L/423E, 302L/423E/605K/621R, 302L/543V/621R/696Q/699Q, 302L/605K/621R/721R, 312Q/331A/451G/621R/696Q/699Q, 312Q/423E/621R, 312Q/510R/605K, 331A/423E/527F, 331A/525R/605K/699Q, 331A/605K/699Q, 331S/423E, 331S/451G, 340T/451G/494V/525R, 358N/467N/489R/699Q, 358N/467N/699Q, 358N/467N/699Q/814T, 423E/443D/451G/494S/510R, 423E/451G/605K/659G/699Q, 423E/489R/494S/529D/605K/621R/696Q/699Q, 423E/494S, 423E/525R, 423E/605K/621R, 451G/510R, 467N, 467N/489R, 467N/489R/510R/543V/699S/814T, 467N/489R/699Q, 467N/489R/699S/814T, 467N/489R/814T, 467N/510R/699Q, 467N/510R/699Q/814T, 467N/510R/699S/814T, 467N/510R/814T, 467N/699Q, 467N/699S, 484V, 489R, 489R/494S/605K/721R, 489R/510R/525R/605K/699Q, 489R/699Q, 489R/699Q/814T, 489R/699S, 494S/659G, 494S/699S, 494V/605K/699Q, 498M, 498T, 503I, 510R, 510R/605K/659G, 510R/699Q, 510R/699Q/814T, 510R/699S/814T, 524D, 524Q, 526Q, 527R, 531V, 540S, 542R, 543V/699Q, 543V/699Q/814T, 543V/699S/814T, 547M, 569V, 577S/614C, 605K, 605K/621R/659G/696Q/699S, 605K/621R/696Q/699S, 621R, 659G/699Q, 673K, 684P, 685K, 685K/714T, 685R, 699Q, 699Q/814T, 702K, and 716R, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/K227E/E312Q/A423E/A525R/R605K/A699Q, S45L/R268Q/P302L/A423E/N494S/S621R/A699S, S45L/R268Q/E443D/A525R/E696Q, S45L/L331A/H489R, S45L/L331A/S621R, S45L/R340T/A423E, S45L/N494V/A525R/S621R/E696Q/A699Q, S45L/R605K, S45L/E696Q/A699Q, E65K/F685R, K227E, K227E/R268G/A423E/S621R, K227E/R268Q/R340T/A451G, K227E/P302L, K227E/E312Q/H489R/N494S/S621R, K227E/L331A/A423E/R605K, K227E/L331S, K227E/L331S/R605K/S659G, K227E/R340T/A423E/R605K, K227E/R340T/S621R, K227E/A423E, K227E/A423E/A451G, K227E/A451G/A525R/S621R/S659G/A699Q/E721R, K227E/A525R, K227E/Q543V/S659G/A699S, K227E/R605K/E721R, K227E/A699S, R268G/P302L/A423E/R605K, R268G/R340T, R268G/A423E/R478H/H489R/A525R/R605K/A699Q, R268G/A423E/P510R/R605K, R268G/A423E/A699Q, R268G/S621R, R268Q/P302L/R340T/R358N/R605K/A699Q, R268Q/R340T/A423E/E443D/A525R/S621R/S659G, R268Q/R340T/A423E/S621R, R268Q/R340T/A525R/R605K, R268Q/A423E, R268Q/A423E/H489R/N494S/R605K/S659G, R268Q/A423E/R605K/A699S, R268Q/A467N, R268Q/A467N/H489R, R268Q/A467N/H489R/P510R/A699Q, R268Q/A467N/H489R/A699Q, R268Q/A467N/H489R/A699S/E814T, R268Q/A467N/P510R/A699S/E814T, R268Q/H489R/N494S/R605K/A699Q, R268Q/H489R/A699Q, R268Q/P510R, R268Q/P510R/A699Q/E814T, R268Q/A525R, R268Q/A525R/R605K, R268Q/Q543V, R268Q/Q543V/A699Q, R268Q/R605K/S621R/E721R, R268Q/A699Q, R268Q/A699S, P302L/A423E, P302L/A423E/R605K/S621R, P302L/Q543V/S621R/E696Q/A699Q, P302L/R605K/S621R/E721R, E312Q/L331A/A451G/S621R/E696Q/A699Q, E312Q/A423E/S621R, E312Q/P510R/R605K, L331A/A423E/V527F, L331A/A525R/R605K/A699Q, L331A/R605K/A699Q, L331S/A423E, L331S/A451G, R340T/A451G/N494V/A525R, R358N/A467N/H489R/A699Q, R358N/A467N/A699Q, R358N/A467N/A699Q/E814T, A423E/E443D/A451G/N494S/P510R, A423E/A451G/R605K/S659G/A699Q, A423E/H489R/N494S/E529D/R605K/S621R/E696Q/A699Q, A423E/N494S, A423E/A525R, A423E/R605K/S621R, A451G/P510R, A467N, A467N/H489R, A467N/H489R/P510R/Q543V/A699S/E814T, A467N/H489R/A699Q, A467N/H489R/A699S/E814T, A467N/H489R/E814T, A467N/P510R/A699Q, A467N/P510R/A699Q/E814T, A467N/P510R/A699S/E814T, A467N/P510R/E814T, A467N/A699Q, A467N/A699S, F484V, H489R, H489R/N494S/R605K/E721R, H489R/P510R/A525R/R605K/A699Q, H489R/A699Q, H489R/A699Q/E814T, H489R/A699S, N494S/S659G, N494S/A699S, N494V/R605K/A699Q, Q498M, Q498T, L503I, P510R, P510R/R605K/S659G, P510R/A699Q, P510R/A699Q/E814T, P510R/A699S/E814T, S524D, S524Q, A526Q, V527R, L531V, K540S, L542R, Q543V/A699Q, Q543V/A699Q/E814T, Q543V/A699S/E814T, L547M, L569V, A577S/R614C, R605K, R605K/S621R/S659G/E696Q/A699S, R605K/S621R/E696Q/A699S, S621R, S659G/A699Q, T673K, A684P, F685K, F685K/A714T, F685R, A699Q, A699Q/E814T, I702K, and I716R, or combinations thereof, wherein the amino acid positions are relative to the reference sequence of SEQ ID NO: 2310.

In some embodiments, the engineered DNA polymerase comprises one or more substitutions or substitution sets at amino acid positions selected from 18/268/302/312/605/659/699, 45/227/268/423/489/510/605, 45/227/302/423/510/605/621/699, 45/227/312/423/525/605/699, 45/268/302/423/494/621/699, 45/494/525/621/696/699, 227, 227/268/340/451, 227/268/423/621, 227/302, 227/312/489/494/621, 227/331, 227/331/423/605, 227/331/605/659, 227/340/423/605, 227/340/621, 227/423, 227/543/659/699, 227/605/721, 268, 268/302, 268/302/423/605, 268/340/423/443/525/621/659, 268/423/510/605, 268/423/605/699, 268/467, 268/467/489, 268/467/489/510/699, 268/467/489/699, 268/489/699, 268/510, 268/510/699/814, 268/525/605, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/605, 331/605/699, 340/451/494/525, 358/467/489/699, 358/467/699/814, 423, 423/443/451/494/510, 423/489/494/529/605/621/696/699, 423/605/621, 451/510, 467/489, 467/489/510/543/699/814, 467/489/699, 467/489/814, 467/510/699, 467/510/699/814, 467/699, 467/699/814, 484, 489, 489/510/525/605/699, 489/699, 489/699/814, 494/605/699, 498, 503, 510, 510/605/659, 510/699, 510/699/814, 524, 526, 543/699, 543/699/814, 605/621/659/696/699, 605/621/696/699, 621, and 699/814, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/227E/312Q/423E/525R/605K/699Q, 45L/268Q/302L/423E/494S/621R/699S, 45L/494V/525R/621R/696Q/699Q, 227E, 227E/268G/423E/621R, 227E/268Q/340T/451G, 227E/302L, 227E/312Q/489R/494S/621R, 227E/331A/423E/605K, 227E/331S, 227E/331S/605K/659G, 227E/340T/423E/605K, 227E/340T/621R, 227E/423E, 227E/543V/659G/699S, 227E/605K/721R, 268G/302L/423E/605K, 268G/423E/510R/605K, 268G/621R, 268Q, 268Q/302L, 268Q/340T/423E/443D/525R/621R/659G, 268Q/423E/605K/699S, 268Q/467N, 268Q/467N/489R, 268Q/467N/489R/510R/699Q, 268Q/467N/489R/699Q, 268Q/489R/699Q, 268Q/510R, 268Q/510R/699Q/814T, 268Q/525R/605K, 268Q/543V/699Q, 268Q/605K/621R/721R, 268Q/699Q, 302L/423E, 302L/423E/605K/621R, 302L/543V/621R/696Q/699Q, 302L/605K/621R/721R, 312Q/331A/451G/621R/696Q/699Q, 312Q/423E/621R, 312Q/510R/605K, 331A/423E/527F, 331A/605K, 331A/605K/699Q, 331S/423E, 331S/605K, 340T/451G/494V/525R, 358N/467N/489R/699Q, 358N/467N/699Q/814T, 423E, 423E/443D/451G/494S/510R, 423E/489R/494S/529D/605K/621R/696Q/699Q, 423E/605K/621R, 451G/510R, 467N/489R, 467N/489R/510R/543V/699S/814T, 467N/489R/699Q, 467N/489R/814T, 467N/510R/699Q, 467N/510R/699Q/814T, 467N/510R/699S/814T, 467N/699Q, 467N/699Q/814T, 467N/699S, 484V, 489R, 489R/510R/525R/605K/699Q, 489R/699Q, 489R/699Q/814T, 494V/605K/699Q, 498M, 498T, 5031, 510R, 510R/605K/659G, 510R/699Q, 510R/699Q/814T, 510R/699S/814T, 524D, 524Q, 526Q, 543V/699Q, 543V/699Q/814T, 605K/621R/659G/696Q/699S, 605K/621R/696Q/699S, 621R, and 699Q/814T, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/K227E/E312Q/A423E/A525R/R605K/A699Q, S45L/R268Q/P302L/A423E/N494S/S621R/A699S, S45L/N494V/A525R/S621R/E696Q/A699Q, K227E, K227E/R268G/A423E/S621R, K227E/R268Q/R340T/A451G, K227E/P302L, K227E/E312Q/H489R/N494S/S621R, K227E/L331A/A423E/R605K, K227E/L331S, K227E/L331S/R605K/S659G, K227E/R340T/A423E/R605K, K227E/R340T/S621R, K227E/A423E, K227E/Q543V/S659G/A699S, K227E/R605K/E721R, R268G/P302L/A423E/R605K, R268G/A423E/P510R/R605K, R268G/S621R, R268Q, R268Q/P302L, R268Q/R340T/A423E/E443D/A525R/S621R/S659G, R268Q/A423E/R605K/A699S, R268Q/A467N, R268Q/A467N/H489R, R268Q/A467N/H489R/P510R/A699Q, R268Q/A467N/H489R/A699Q, R268Q/H489R/A699Q, R268Q/P510R, R268Q/P510R/A699Q/E814T, R268Q/A525R/R605K, R268Q/Q543V/A699Q, R268Q/R605K/S621R/E721R, R268Q/A699Q, P302L/A423E, P302L/A423E/R605K/S621R, P302L/Q543V/S621R/E696Q/A699Q, P302L/R605K/S621R/E721R, E312Q/L331A/A451G/S621R/E696Q/A699Q, E312Q/A423E/S621R, E312Q/P510R/R605K, L331A/A423E/V527F, L331A/R605K, L331A/R605K/A699Q, L331S/A423E, L331S/R605K, R340T/A451G/N494V/A525R, R358N/A467N/H489R/A699Q, R358N/A467N/A699Q/E814T, A423E, A423E/E443D/A451G/N494S/P510R, A423E/H489R/N494S/E529D/R605K/S621R/E696Q/A699Q, A423E/R605K/S621R, A451G/P510R, A467N/H489R, A467N/H489R/P510R/Q543V/A699S/E814T, A467N/H489R/A699Q, A467N/H489R/E814T, A467N/P510R/A699Q, A467N/P510R/A699Q/E814T, A467N/P510R/A699S/E814T, A467N/A699Q, A467N/A699Q/E814T, A467N/A699S, F484V, H489R, H489R/P510R/A525R/R605K/A699Q, H489R/A699Q, H489R/A699Q/E814T, N494V/R605K/A699Q, Q498M, Q498T, L503I, P510R, P510R/R605K/S659G, P510R/A699Q, P510R/A699Q/E814T, P510R/A699S/E814T, S524D, S524Q, A526Q, Q543V/A699Q, Q543V/A699Q/E814T, R605K/S621R/S659G/E696Q/A699S, R605K/S621R/E696Q/A699S, S621R, and A699Q/E814T, or combinations thereof, wherein the amino acid positions are relative to the reference sequence of SEQ ID NO: 2310.

In some embodiments, the engineered DNA polymerase comprises one or more substitutions or substitution sets at amino acid positions selected from 18/268/302/312/605/659/699, 45/227/268/423/489/510/605, 45/227/302/423/510/605/621/699, 45/494/525/621/696/699, 227/331/605/659, 227/605/721, 268, 268/302/423/605, 268/423/605/699, 268/467, 268/467/489, 268/467/489/510/699, 268/467/489/699, 268/489/699, 268/510, 268/605/621/721, 302/543/621/696/699, 312/331/451/621/696/699, 312/510/605, 331/423/527, 340/451/494/525, 423/489/494/529/605/621/696/699, 423/605/621, 467/489/510/543/699/814, 467/489/699, 467/489/814, 467/510/699/814, 489/510/525/605/699, 489/699, 489/699/814, 494/605/699, 510, 510/699/814, 543/699, and 543/699/814, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/494V/525R/621R/696Q/699Q, 227E/331S/605K/659G, 227E/605K/721R, 268G/302L/423E/605K, 268Q, 268Q/423E/605K/699S, 268Q/467N, 268Q/467N/489R, 268Q/467N/489R/510R/699Q, 268Q/467N/489R/699Q, 268Q/489R/699Q, 268Q/510R, 268Q/605K/621R/721R, 302L/543V/621R/696Q/699Q, 312Q/331A/451G/621R/696Q/699Q, 312Q/510R/605K, 331A/423E/527F, 340T/451G/494V/525R, 423E/489R/494S/529D/605K/621R/696Q/699Q, 423E/605K/621R, 467N/489R/510R/543V/699S/814T, 467N/489R/699Q, 467N/489R/814T, 467N/510R/699Q/814T, 467N/510R/699S/814T, 489R/510R/525R/605K/699Q, 489R/699Q, 489R/699Q/814T, 494V/605K/699Q, 510R, 510R/699Q/814T, 510R/699S/814T, 543V/699Q, and 543V/699Q/814T, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/N494V/A525R/S621R/E696Q/A699Q, K227E/L331S/R605K/S659G, K227E/R605K/E721R, R268G/P302L/A423E/R605K, R268Q, R268Q/A423E/R605K/A699S, R268Q/A467N, R268Q/A467N/H489R, R268Q/A467N/H489R/P510R/A699Q, R268Q/A467N/H489R/A699Q, R268Q/H489R/A699Q, R268Q/P510R, R268Q/R605K/S621R/E721R, P302L/Q543V/S621R/E696Q/A699Q, E312Q/L331A/A451G/S621R/E696Q/A699Q, E312Q/P510R/R605K, L331A/A423E/V527F, R340T/A451G/N494V/A525R, A423E/H489R/N494S/E529D/R605K/S621R/E696Q/A699Q, A423E/R605K/S621R, A467N/H489R/P510R/Q543V/A699S/E814T, A467N/H489R/A699Q, A467N/H489R/E814T, A467N/P510R/A699Q/E814T, A467N/P510R/A699S/E814T, H489R/P510R/A525R/R605K/A699Q, H489R/A699Q, H489R/A699Q/E814T, N494V/R605K/A699Q, P510R, P510R/A699Q/E814T, P510R/A699S/E814T, Q543V/A699Q, and Q543V/A699Q/E814T, or combinations thereof, wherein the amino acid positions are relative to the reference sequence of SEQ ID NO: 2310.

In some embodiments, the engineered DNA polymerase comprises one or more substitutions or substitution sets at amino acid positions selected from 18/268/302/312/605/659/699, 45/227/268/423/489/510/605, 45/227/302/423/510/605/621/699, 65/685, 227/331/605/659, 227/605/721, 268, 268/302/423/605, 268/423/605/699, 268/467, 268/467/489, 268/467/489/699, 268/489/699, 268/510, 268/605/621/721, 302/543/621/696/699, 312/331/451/621/696/699, 312/510/605, 423/605/621, 467/489/510/543/699/814, 467/489/699, 467/489/814, 467/510/699/814, 489/699, 489/699/814, 510, 510/699/814, 542, 543/699, 543/699/814, 685, and 685/714, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 65K/685R, 227E/331S/605K/659G, 227E/605K/721R, 268G/302L/423E/605K, 268Q, 268Q/423E/605K/699S, 268Q/467N, 268Q/467N/489R, 268Q/467N/489R/699Q, 268Q/489R/699Q, 268Q/510R, 268Q/605K/621R/721R, 302L/543V/621R/696Q/699Q, 312Q/331A/451G/621R/696Q/699Q, 312Q/510R/605K, 423E/605K/621R, 467N/489R/510R/543V/699S/814T, 467N/489R/699Q, 467N/489R/814T, 467N/510R/699Q/814T, 467N/510R/699S/814T, 489R/699Q, 489R/699Q/814T, 510R, 510R/699S/814T, 542R, 543V/699Q, 543V/699Q/814T, 685K, 685K/714T, and 685, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, E65K/F685R, K227E/L331S/R605K/S659G, K227E/R605K/E721R, R268G/P302L/A423E/R605K, R268Q, R268Q/A423E/R605K/A699S, R268Q/A467N, R268Q/A467N/H489R, R268Q/A467N/H489R/A699Q, R268Q/H489R/A699Q, R268Q/P510R, R268Q/R605K/S621R/E721R, P302L/Q543V/S621R/E696Q/A699Q, E312Q/L331A/A451G/S621R/E696Q/A699Q, E312Q/P510R/R605K, A423E/R605K/S621R, A467N/H489R/P510R/Q543V/A699S/E814T, A467N/H489R/A699Q, A467N/H489R/E814T, A467N/P510R/A699Q/E814T, A467N/P510R/A699S/E814T, H489R/A699Q, H489R/A699Q/E814T, P510R, P510R/A699S/E814T, L542R, Q543V/A699Q, Q543V/A699Q/E814T, F685K, F685K/A714T, and F685R, or combinations thereof, wherein the amino acid positions are relative to the reference sequence of SEQ ID NO: 2310.

The present invention also provides engineered DNA polymerases or a functional fragment thereof, comprising a polypeptide sequence having at least 75% or more sequence identity to a reference sequence comprising residues 12 to 841 of SEQ ID NO: 2994, or to a reference sequence of SEQ ID NO: 2994, wherein the polypeptide sequence comprises one or more substitutions relative to the reference sequence comprising residues 12 to 841 of SEQ ID NO: 2994, or to the reference sequence of SEQ ID NO: 2994.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2994 or to the reference sequence of SEQ ID NO: 2994.

In some embodiments, the engineered DNA polymerase comprises one or more substitutions or substitution sets at amino acid positions selected from 4, 6, 10, 11, 12, 14, 14/50, 14/50/56/598/725, 14/50/598/695, 14/95, 14/95/501/642, 14/139/249/433, 14/139/725, 14/167, 14/167/197/433, 14/167/197/501/725, 14/167/197/642/695, 14/167/249/433/598/642, 14/167/249/501/598/642, 14/167/249/598/695/725, 14/167/249/642, 14/167/642, 14/167/695, 14/167/725, 14/197/249/642, 14/197/642, 14/249/433/725, 14/249/598, 14/249/642, 14/249/725, 14/433/598, 14/501, 14/598, 14/598/695, 14/598/695/725, 14/642/725, 18, 18/302, 18/302/531/673/684, 18/302/547, 18/484/673, 18/484/673/684, 18/524/547, 18/547, 18/684, 29, 45/268/299/302/321/621/659, 45/268/302/621/721, 45/376/385/408/494/525/621/696, 45/423, 48, 50, 50/433/598, 50/501, 50/598, 50/598/695, 50/642, 53, 76, 77, 91, 95/167/197/598/642/695, 95/167/197/598/695/725, 95/249/433/642, 95/249/598, 95/598, 99, 105, 122, 139/167, 139/167/197/433/501/642, 139/433/598, 141, 167, 167/197, 167/197/642/695/725, 167/642, 167/725, 177, 197/642/725, 197/695, 202, 204, 209, 217, 227, 239, 247, 249/695, 249/725, 266, 268, 268/302/451/494/621, 270, 291, 291/684, 299, 299/474, 302, 302/423/451/494/621, 302/423/621, 302/451/494, 313, 346, 349, 356, 361, 370, 374, 377, 394, 404, 423, 423/451/621/696, 423/494/525, 426, 426/798, 427, 433/501/725, 436, 439/509/789, 440, 444, 445, 445/446, 446/811, 448, 448/793, 459, 461, 482, 484, 484/684, 485, 489, 494, 498/684, 509, 514, 517, 525/529/621, 533, 543/621/721, 547, 565, 574, 596, 598, 598/642, 598/642/695, 598/695, 598/725, 601, 610, 621, 621/659, 636, 642, 642/692, 659, 662, 673, 683, 684, 688, 689, 690, 695, 725, 741, 744, 744/793, 751, 801, 810, 818, 830, and 832, or combinations thereof, wherein the amino acid positions are relative to the reference sequence of SEQ ID NO: 2994.

In some embodiments, the engineered DNA polymerase comprises one or more substitutions or substitution sets selected from 4A, 4F, 4G, 4R, 4T, 4V, 6F, 6G, 6N, 6R, 6V, 10I, 10Q, 10R, 11E, 11L, 11R, 12D, 12W, 14E, 14E/50H, 14E/50H/56V/598K/725H, 14E/50H/598K/695K, 14E/95D, 14E/95D/501K/642E, 14E/139D/249E/433K, 14E/139D/725H, 14E/167D, 14E/167D/197K/433K, 14E/167D/197K/501K/725H, 14E/167D/197K/642E/695K, 14E/167D/249E/433K/598K/642E, 14E/167D/249E/501K/598K/642E, 14E/167D/249E/598K/695K/725H, 14E/167D/249E/642E, 14E/167D/642E, 14E/167D/695K, 14E/167D/725H, 14E/197K/249E/642E, 14E/197K/642E, 14E/249E/433K/725H, 14E/249E/598K, 14E/249E/642E, 14E/249E/725H, 14E/433K/598K, 14E/501K, 14E/598K, 14E/598K/695K, 14E/598K/695K/725H, 14E/642E/725H, 18H, 18H/302V, 18H/302V/531V/673K/684P, 18H/302V/547M, 18H/484V/673K, 18H/484V/673K/684P, 18H/524D/547M, 18H/547M, 18H/684P, 29M, 29N, 29V, 29W, 45L/268G/299N/302L/321T/621R/659G, 45L/268G/302L/621R/721R, 45L/376F/385F/408A/494V/525R/621R/696Q, 45L/423E, 48M, 48V, 50H, 50H/433K/598K, 50H/501K, 50H/598K, 50H/598K/695K, 50H/642E, 53A, 53Q, 53W, 76L, 76S, 76W, 76Y, 77H, 77M, 77R, 77S, 91C, 91R, 91S, 91W, 95D/167D/197K/598K/642E/695K, 95D/167D/197K/598K/695K/725H, 95D/249E/433K/642E, 95D/249E/598K, 95D/598K, 99G, 99L, 99P, 99S, 99T, 105E, 105F, 105G, 105L, 105S, 105T, 122G, 122I, 122L, 122N, 122R, 122S, 139D/167D, 139D/167D/197K/433K/501K/642E, 139D/433K/598K, 141C, 167D, 167D/197K, 167D/197K/642E/695K/725H, 167D/642E, 167D/725H, 177C, 177L, 177Q, 177R, 197K/642E/725H, 197K/695K, 202G, 204L, 209G, 209M, 209N, 209V, 217G, 217R, 217T, 227M, 227S, 239I, 239L, 239W, 239Y, 247A, 247G, 249E/695K, 249E/725H, 266E, 268G, 268G/302V/451G/494S/621R, 268H, 268S, 270P, 291Q, 291Q/684P, 299E, 299F, 299G, 299K, 299M, 299P, 299R/474V, 299V, 302V, 302V/423E/451G/494S/621R, 302V/423E/621R, 302V/451G/494S, 313F, 313S, 313T, 313V, 346H, 346M, 346P, 346R, 346T, 349N, 349P, 349R, 349T, 349V, 349W, 356A, 356G, 356I, 356Q, 356R, 356V, 361G, 370D, 370T, 370Y, 374G, 377G, 377L, 394C, 394D, 394G, 394M, 394R, 404C, 404K, 404R, 404S, 404V, 423E, 423E/451G/621R/696Q, 423E/494V/525R, 423F, 423G, 423R, 423S, 423V, 426A, 426G, 426H, 426Q, 426R/798F, 426V, 426W, 427A, 427C, 427E, 427K, 427L, 427M, 427R, 427S, 427T, 427W, 433K/501K/725H, 436E, 436G, 436H, 436K, 436S, 436W, 436Y, 439F/509V/7891, 440C, 440G, 440W, 444T, 444V, 445G/446V, 445H, 446M/811I, 448G, 448K, 448Q, 448S, 448T, 448V/793P, 459F, 459L, 459R, 461A, 482A, 484V, 484V/684P, 485C, 485D, 485F, 485M, 485N, 485Q, 485S, 485T, 489S, 489W, 494V, 498M/684P, 509A, 514R, 514S, 517I, 517N, 517R, 517S, 517T, 525R/529D/621R, 533C, 533R, 543V/621R/721R, 547M, 565C, 565R, 565S, 565T, 574L, 574Q, 596A, 596V, 598K, 598K/642E, 598K/642E/695K, 598K/695K, 598K/725H, 601S, 610A, 621R, 621R/659G, 636G, 642E, 642E/692D, 659A, 659G, 659N, 659R, 659T, 662D, 662F, 662H, 662Q, 662T, 662V, 662W, 673K, 683K, 684P, 688A, 688C, 688G, 689G, 690G, 690R, 690S, 695K, 725H, 741G, 741S, 744C, 744G/793P, 744L, 744Q, 744R, 744W, 751G, 801S, 801T, 810C, 810D, 810F, 810G, 810N, 818A, 818E, 818F, 818L, 818R, 818S, 830M, 832F, 832L, and 832Y, or combinations thereof, wherein the amino acid positions are relative to the reference sequence of SEQ ID NO: 2994.

In some embodiments, the engineered DNA polymerase comprises one or more substitutions or substitution sets selected from H4A, H4F, H4G, H4R, H4T, H4V, H6F, H6G, H6N, H6R, H6V, S10I, S10Q, S10R, G11E, G11L, G11R, V12D, V12W, P14E, P14E/Q50H, P14E/Q50H/A56V/P598K/T725H, P14E/Q50H/P598K/Y695K, P14E/P95D, P14E/P95D/R501K/Q642E, P14E/R139D/K249E/E433K, P14E/R139D/T725H, P14E/E167D, P14E/E167D/P197K/E433K, P14E/E167D/P197K/R501K/T725H, P14E/E167D/P197K/Q642E/Y695K, P14E/E167D/K249E/E433K/P598K/Q642E, P14E/E167D/K249E/R501K/P598K/Q642E, P14E/E167D/K249E/P598K/Y695K/T725H, P14E/E167D/K249E/Q642E, P14E/E167D/Q642E, P14E/E167D/Y695K, P14E/E167D/T725H, P14E/P197K/K249E/Q642E, P14E/P197K/Q642E, P14E/K249E/E433K/T725H, P14E/K249E/P598K, P14E/K249E/Q642E, P14E/K249E/T725H, P14E/E433K/P598K, P14E/R501K, P14E/P598K, P14E/P598K/Y695K, P14E/P598K/Y695K/T725H, P14E/Q642E/T725H, P18H, P18H/P302V, P18H/P302V/L531V/T673K/A684P, P18H/P302V/L547M, P18H/F484V/T673K, P18H/F484V/T673K/A684P, P18H/S524D/L547M, P18H/L547M, P18H/A684P, H29M, H29N, H29V, H29W, S45L/Q268G/S299N/P302L/S321T/S621R/S659G, S45L/Q268G/P302L/S621R/E721R, S45L/L376F/L385F/T408A/N494V/A525R/S621R/E696Q, S45L/A423E, P48M, P48V, Q50H, Q50H/E433K/P598K, Q50H/R501K, Q50H/P598K, Q50H/P598K/Y695K, Q50H/Q642E, Y53A, Y53Q, Y53W, A76L, A76S, A76W, A76Y, K77H, K77M, K77R, K77S, A91C, A91R, A91S, A91W, P95D/E167D/P197K/P598K/Q642E/Y695K, P95D/E167D/P197K/P598K/Y695K/T725H, P95D/K249E/E433K/Q642E, P95D/K249E/P598K, P95D/P598K, D99G, D99L, D99P, D99S, D99T, A105E, A105F, A105G, A105L, A105S, A105T, P122G, P122I, P122L, P122N, P122R, P122S, R139D/E167D, R139D/E167D/P197K/E433K/R501K/Q642E, R139D/E433K/P598K, G141C, E167D, E167D/P197K, E167D/P197K/Q642E/Y695K/T725H, E167D/Q642E, E167D/T725H, W177C, W177L, W177Q, W177R, P197K/Q642E/T725H, P197K/Y695K, P202G, V204L, E209G, E209M, E209N, E209V, K217G, K217R, K217T, K227M, K227S, E239I, E239L, E239W, E239Y, D247A, D247G, K249E/Y695K, K249E/T725H, D266E, Q268G, Q268G/P302V/A451G/N494S/S621R, Q268H, Q268S, R270P, L291Q, L291Q/A684P, S299E, S299F, S299G, S299K, S299M, S299P, S299R/A474V, S299V, P302V, P302V/A423E/A451G/N494S/S621R, P302V/A423E/S621R, P302V/A451G/N494S, G313F, G313S, G313T, G313V, D346H, D346M, D346P, D346R, D346T, G349N, G349P, G349R, G349T, G349V, G349W, E356A, E356G, E356I, E356Q, E356R, E356V, L361G, L370D, L370T, L370Y, L374G, P377G, P377L, T394C, T394D, T394G, T394M, T394R, G404C, G404K, G404R, G404S, G404V, A423E, A423E/A451G/S621R/E696Q, A423E/N494V/A525R, A423F, A423G, A423R, A423S, A423V, L426A, L426G, L426H, L426Q, L426R/L798F, L426V, L426W, G427A, G427C, G427E, G427K, G427L, G427M, G427R, G427S, G427T, G427W, E433K/R501K/T725H, L436E, L436G, L436H, L436K, L436S, L436W, L436Y, Y439F/L509V/L7891, E440C, E440G, E440W, K444T, K444V, P445G/L446V, P445H, L446M/L811I, R448G, R448K, R448Q, R448S, R448T, R448V/H793P, K459F, K459L, K459R, D461A, E482A, F484V, F484V/A684P, R485C, R485D, R485F, R485M, R485N, R485Q, R485S, R485T, R489S, R489W, N494V, Q498M/A684P, L509A, K514R, K514S, K517I, K517N, K517R, K517S, K517T, A525R/E529D/S621R, E533C, E533R, Q543V/S621R/E721R, L547M, K565C, K565R, K565S, K565T, N574L, N574Q, R596A, R596V, P598K, P598K/Q642E, P598K/Q642E/Y695K, P598K/Y695K, P598K/T725H, Q601S, E610A, S621R, S621R/S659G, N636G, Q642E, Q642E/G692D, S659A, S659G, S659N, S659R, S659T, A662D, A662F, A662H, A662Q, A662T, A662V, A662W, T673K, S683K, A684P, S688A, S688C, S688G, Q689G, E690G, E690R, E690S, Y695K, T725H, D741G, D741S, A744C, A744G/H793P, A744L, A744Q, A744R, A744W, E751G, P801S, P801T, A810C, A810D, A810F, A810G, A810N, G818A, G818E, G818F, G818L, G818R, G818S, V830M, 1832F, 1832L, and I832Y, or combinations thereof, wherein the amino acid positions are relative to the reference sequence of SEQ ID NO: 2994.

In some embodiments, the engineered DNA polymerase comprises one or more substitutions or substitution sets at positions selected from 4, 6, 10, 11, 14, 14/50, 14/139/725, 14/167, 14/167/642, 14/167/695, 14/598, 18/302/531/673/684, 18/524/547, 18/684, 45/268/299/302/321/621/659, 45/268/302/621/721, 48, 50/598, 77, 95/167/197/598/695/725, 99, 105, 122, 167, 167/197, 177, 209, 217, 239, 249/695, 268, 346, 349, 356, 423, 423/451/621/696, 426, 426/798, 427, 433/501/725, 436, 439/509/789, 445, 448, 448/793, 482, 484, 484/684, 514, 517, 533, 543/621/721, 565, 574, 598/695, 621, 621/659, 659, 662, 684, 688, 690, 725, 744/793, 801, 810, and 832, or combinations thereof, wherein the amino acid positions are relative to the reference sequence of SEQ ID NO: 2994.

In some embodiments, the engineered DNA polymerase comprises one or more substitutions or substitution sets selected from 4G, 6G, 6R, 101, 11E, 11R, 14E, 14E/50H, 14E/139D/725H, 14E/167D, 14E/167D/642E, 14E/167D/695K, 14E/598K, 18H/302V/531V/673K/684P, 18H/524D/547M, 18H/684P, 45L/268G/299N/302L/321T/621R/659G, 45L/268G/302L/621R/721R, 48V, 50H/598K, 77M, 95D/167D/197K/598K/695K/725H, 99S, 99T, 105F, 105T, 122G, 122R, 167D, 167D/197K, 177L, 209V, 217G, 2391, 249E/695K, 268S, 346P, 346R, 349P, 349R, 349V, 356V, 423E/451G/621R/696Q, 423G, 423S, 426G, 426H, 426R/798F, 426W, 427A, 427C, 427L, 427R, 433K/501K/725H, 436E, 436K, 436S, 439F/509V/7891, 445H, 448S, 448V/793P, 482A, 484V, 484V/684P, 514R, 514S, 5171, 517T, 533C, 543V/621R/721R, 565R, 574L, 598K/695K, 621R, 621R/659G, 659G, 659R, 662H, 662T, 662W, 684P, 688A, 688C, 688G, 690G, 725H, 744G/793P, 801T, 810D, 810F, 810N, and 832L, or combinations thereof, wherein the amino acid positions are relative to the reference sequence of SEQ ID NO: 2994.

In some embodiments, the engineered DNA polymerase comprises one or more substitutions or substitution sets selected from H4G, H6G, H6R, S10I, G11E, G11R, P14E, P14E/Q50H, P14E/R139D/T725H, P14E/E167D, P14E/E167D/Q642E, P14E/E167D/Y695K, P14E/P598K, P18H/P302V/L531V/T673K/A684P, P18H/S524D/L547M, P18H/A684P, S45L/Q268G/S299N/P302L/S321T/S621R/S659G, S45L/Q268G/P302L/S621R/E721R, P48V, Q50H/P598K, K77M, P95D/E167D/P197K/P598K/Y695K/T725H, D99S, D99T, A105F, A105T, P122G, P122R, E167D, E167D/P197K, W177L, E209V, K217G, E239I, K249E/Y695K, Q268S, D346P, D346R, G349P, G349R, G349V, E356V, A423E/A451G/S621R/E696Q, A423G, A423S, L426G, L426H, L426R/L798F, L426W, G427A, G427C, G427L, G427R, E433K/R501K/T725H, L436E, L436K, L436S, Y439F/L509V/L789I, P445H, R448S, R448V/H793P, E482A, F484V, F484V/A684P, K514R, K514S, K517I, K517T, E533C, Q543V/S621R/E721R, K565R, N574L, P598K/Y695K, S621R, S621R/S659G, S659G, S659R, A662H, A662T, A662W, A684P, S688A, S688C, S688G, E690G, T725H, A744G/H793P, P801T, A810D, A810F, A810N, and I832L, or combinations thereof, wherein the amino acid positions are relative to the reference sequence of SEQ ID NO: 2994.

In some embodiments, the engineered DNA polymerase comprises one or more substitutions or substitution sets at amino acid positions selected from 11, 12, 18/547, 45/268/299/302/321/621/659, 45/268/302/621/721, 45/376/385/408/494/525/621/696, 48, 105, 122, 302, 346, 426/798, 439/509/789, 448/793, 484, 514, 525/529/621, 533, 543/621/721, 574, 690, and 744/793, or combinations thereof, wherein the amino acid positions are relative to the reference sequence of SEQ ID NO: 2994.

In some embodiments, the engineered DNA polymerase comprises one or more substitutions or substitution sets selected from 11E, 12W, 18H/547M, 45L/268G/299N/302L/321T/621R/659G, 45L/268G/302L/621R/721R, 45L/376F/385F/408A/494V/525R/621R/696Q, 48M, 105F, 122G, 302V, 346R, 426R/798F, 439F/509V/7891, 448V/793P, 484V, 514S, 525R/529D/621R, 533C, 543V/621R/721R, 574Q, 690G, and 744G/793P, or combinations thereof, wherein the amino acid positions are relative to the reference sequence of SEQ ID NO: 2994.

In some embodiments, the engineered DNA polymerase comprises one or more substitutions or substitution sets selected from G11E, V12W, P18H/L547M, S45L/Q268G/S299N/P302L/S321T/S621R/S659G, S45L/Q268G/P302L/S621R/E721R, S45L/L376F/L385F/T408A/N494V/A525R/S621R/E696Q, P48M, A105F, P122G, P302V, D346R, L426R/L798F, Y439F/L509V/L789I, R448V/H793P, F484V, K514S, A525R/E529D/S621R, E533C, Q543V/S621R/E721R, N574Q, E690G, and A744G/H793P, or combinations thereof, wherein the amino acid positions are relative to the reference sequence of SEQ ID NO: 2994.

In some embodiments, the engineered DNA polymerase comprises one or more substitutions or substitution sets at positions selected from 11, 12, 18, 18/302, 18/302/547, 18/547, 45/268/299/302/321/621/659, 45/268/302/621/721, 45/376/385/408/494/525/621/696, 48, 105, 122, 291/684, 302, 346, 426/798, 439/509/789, 448/793, 484, 514, 525/529/621, 533, 547, 574, 684, 690, and 744/793, or combinations thereof, wherein the amino acid positions are relative to the reference sequence of SEQ ID NO: 2994.

In some embodiments, the engineered DNA polymerase comprises one or more substitutions or substitution sets selected from 11E, 12W, 18H, 18H/302V, 18H/302V/547M, 18H/547M, 45L/268G/299N/302L/321T/621R/659G, 45L/268G/302L/621R/721R, 45L/376F/385F/408A/494V/525R/621R/696Q, 48M, 105F, 122G, 291Q/684P, 302V, 346R, 426R/798F, 439F/509V/789I, 448V/793P, 484V, 514S, 525R/529D/621R, 533C, 547M, 574Q, 684P, 690G, and 744G/793P, or combinations thereof, wherein the amino acid positions are relative to the reference sequence of SEQ ID NO: 2994.

In some embodiments, the engineered DNA polymerase comprises one or more substitutions or substitution sets selected from G11E, V12W, P18H, P18H/P302V, P18H/P302V/L547M, P18H/L547M, S45L/Q268G/S299N/P302L/S321T/S621R/S659G, S45L/Q268G/P302L/S621R/E721R, S45L/L376F/L385F/T408A/N494V/A525R/S621R/E696Q, P48M, A105F, P122G, L291Q/A684P, P302V, D346R, L426R/L798F, Y439F/L509V/L789I, R448V/H793P, F484V, K514S, A525R/E529D/S621R, E533C, L547M, N574Q, A684P, E690G, and A744G/H793P, or combinations thereof, wherein the amino acid positions are relative to the reference sequence of SEQ ID NO: 2994.

The present invention also provides engineered DNA polymerases or a functional fragment thereof, comprising a polypeptide sequence having at least 75% or more sequence identity to a reference sequence comprising residues 12 to 841 of SEQ ID NO: 3796, or to a reference sequence of SEQ ID NO: 3796, wherein the polypeptide sequence comprises one or more substitutions relative to the reference sequence comprising residues 12 to 841 of SEQ ID NO: 3796, or to the reference sequence of SEQ ID NO: 3796.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 3796 or to the reference sequence of SEQ ID NO: 3796.

In some embodiments, the engineered DNA polymerase comprises one or more substitutions or substitution sets at positions selected from 1/2/149, 15/167, 18, 18/105/139/197/249/426/598, 18/105/139/197/291/299/423/426/448/529/547/725, 18/105/249/284/302/423/426/501/673/684/798, 18/105/321/547, 18/139/197/249/299/302/433/547/642/659/695/744, 18/139/197/426/598, 18/139/197/642, 18/139/249/423/433/501/547/725, 18/197/249/291/484, 18/197/321/426/448/451/659/673/744, 18/197/321/433/484, 18/249, 18/249/299/302/423/529/533/642/659/673, 18/249/302/423/484/547/695, 18/249/448/484/673, 18/249/673, 18/291/302/423/684, 18/291/302/433, 18/291/302/525/547/659/684/695, 18/291/302/684, 18/291/423/525/529, 18/291/659, 18/291/695, 18/299/302/423/426/525/529, 18/299/451/525/529/695, 18/302/423/529, 18/302/433/547/684, 18/302/525/529/547, 18/302/529/659/695, 18/321/423, 18/321/484/547/725, 18/423, 18/423/529/695, 18/433, 18/433/673/725, 18/484, 18/598/725, 32, 38/463, 64, 105/139/291/302/423/484/547/725, 105/139/448/501/525/529/642/659, 105/197/249/423/426/547/642/673, 105/321/433/744, 105/321/501, 137, 139/249/291/321/484/695/725, 139/249/291/451/744, 139/249/426/451/501/673/744, 149, 160, 164/172, 167, 172, 197/249/327/423/673/690, 197/302/391/547/598, 197/525/529/659/744, 197/547, 197/684/725, 200/577, 245/423, 247/423/448, 249/321/673/798, 249/423/426/673, 249/448/451/547/659/725, 249/725/744/798, 253, 256, 256/455, 271, 281, 291/302/423, 291/302/529, 291/321/423/725, 291/423/525, 291/423/529/533/598/642/725/798, 291/484/598/642/673/744/798, 291/525, 300, 302, 302/364/376/383/386/408/451/484/598, 302/423/433/529/695, 302/423/525/529, 302/423/547/684/695, 302/684/695, 311, 314, 316, 321/448/529/673, 323, 330, 334/431, 347, 348, 352/363, 353, 354, 358, 361, 363, 368, 372, 376, 376/699, 383, 389, 392, 394, 411, 414, 422, 423/424/547, 423/433/484/529/533/642, 423/433/487/525/547, 423/484/673, 423/529/684, 423/547/598/642, 430, 431, 433/525/529/659/684/695, 436/743, 448/451/529/725, 451/598/725, 455, 455/518, 462, 463, 464, 468, 469, 472, 474, 474/821, 476, 479, 480, 484, 489, 491, 492, 498, 501, 504/548, 505, 510, 519/720, 521, 524, 525, 525/533, 529, 531, 532, 533, 535, 540, 546, 547, 550, 553, 555, 555/572, 557, 558, 559, 566, 568, 570, 571, 572, 573, 574, 575, 576, 577, 583, 588, 590, 590/652, 592, 597, 600, 601, 612, 615, 618, 620, 622, 623, 625, 628, 643, 654, 655, 660, 664, 675, 677, 682, 685, 690, 691/692, 692, 692/766, 697, 703, 705, 708, 711, 715, 720, 726, 728, 730, 732, 738, 743, 746, 752, 753, 756, 766, 769, 770, 779, 793, 800, 802, 803, 805, 813, 819, 825, 827, and 829, or combinations thereof, wherein the amino acid positions are relative to the reference sequence of SEQ ID NO: 3796.

In some embodiments, the engineered DNA polymerase comprises one or more substitutions or substitution sets selected from 1H/2M/149G, 15M/167V, 18H, 18H/105F/139D/197K/249E/426R/598K, 18H/105F/139D/197K/291Q/299N/423E/426R/448V/529D/547M/725H, 18H/105F/249E/284C/302V/423E/426R/501K/673K/684P/798F, 18H/105F/321T/547M, 18H/139D/197K/249E/299N/302V/433K/547M/642E/659G/695K/744G, 18H/139D/197K/426R/598K, 18H/139D/197K/642E, 18H/139D/249E/423E/433K/501K/547M/725H, 18H/197K/249E/291Q/484V, 18H/197K/321T/426R/448V/451G/659G/673K/744G, 18H/197K/321T/433K/484V, 18H/249E, 18H/249E/299N/302V/423E/529D/533C/642E/659G/673K, 18H/249E/302V/423E/484V/547M/695K, 18H/249E/448V/484V/673K, 18H/249E/673K, 18H/291Q/302V/423E/684P, 18H/291Q/302V/433K, 18H/291Q/302V/525R/547M/659G/684P/695K, 18H/291Q/302V/684P, 18H/291Q/423E/525R/529D, 18H/291Q/659G, 18H/291Q/695K, 18H/299N/302V/423E/426R/525R/529D, 18H/299N/451G/525R/529D/695K, 18H/302V/423E/529D, 18H/302V/433K/547M/684P, 18H/302V/525R/529D/547M, 18H/302V/529D/659G/695K, 18H/321T/423E, 18H/321T/484V/547M/725H, 18H/423E, 18H/423E/529D/695K, 18H/433K, 18H/433K/673K/725H, 18H/484V, 18H/598K/725H, 32G, 32L, 32S, 38M/463G, 64G, 105F/139D/291Q/302V/423E/484V/547M/725H, 105F/139D/448V/501K/525R/529D/642E/659G, 105F/197K/249E/423E/426R/547M/642E/673K, 105F/321T/433K/744G, 105F/321T/501K, 137L, 139D/249E/291Q/321T/484V/695K/725H, 139D/249E/291Q/451G/744G, 139D/249E/426R/451G/501K/673K/744G, 149G, 149P, 149V, 160G, 160L, 164V/172F, 167A, 167G, 172H, 172V, 172W, 197K/249E/327L/423E/673K/690G, 197K/302V/391S/547M/598K, 197K/525R/529D/659G/744G, 197K/547M, 197K/684P/725H, 200S/577G, 2451/423L, 247Y/423E/448V, 249E/321T/673K/798F, 249E/423E/426R/673K, 249E/448V/451G/547M/659G/725H, 249E/725H/744G/798F, 253C, 253I, 253M, 256F, 256S, 256S/455F, 256V, 271L, 281V, 291Q/302V/423E, 291Q/302V/529D, 291Q/321T/423E/725H, 291Q/423E/525R, 291Q/423E/529D/533C/598K/642E/725H/798F, 291Q/484V/598K/642E/673K/744G/798F, 291Q/525R, 300G, 302G, 302M, 302S, 302V/364N/376F/383K/386G/408A/451G/484V/598K, 302V/423E/433K/529D/695K, 302V/423E/525R/529D, 302V/423E/547M/684P/695K, 302V/684P/695K, 311C, 311G, 311H, 311K, 314L, 314M, 316N, 321T/448V/529D/673K, 323W, 330M, 330T, 330V, 334T/431C, 347L, 347R, 348A, 352S/363L, 3531, 353T, 354G, 354M, 358E, 358L, 361C, 361R, 361W, 363L, 363T, 363V, 368A, 368L, 368R, 368W, 372K, 372L, 372R, 372S, 372W, 376I, 376K, 376W, 376W/699L, 383I, 383R, 383S, 389G, 389R, 389S, 392G, 392R, 392T, 392V, 394D, 411E, 414S, 422A, 422G, 422L, 422Q, 422R, 423E/424K/547M, 423E/433K/484V/529D/533C/642E, 423E/433K/487V/525R/547M, 423E/484V/673K, 423E/529D/684P, 423E/547M/598K/642E, 430R, 431D, 431Q, 431S, 431T, 431V, 433K/525R/529D/659G/684P/695K, 436F/743K, 448V/451G/529D/725H, 451G/598K/725H, 455E, 455G, 455L, 455P/518I, 455Q, 455S, 455V, 455W, 462A, 462G, 462L, 462Q, 462R, 462S, 462W, 463T, 464H, 464K, 464V, 468A, 468G, 468T, 468W, 469R, 472E, 472G, 472L, 472S, 472T, 474E, 474L/821S, 474R, 474S, 476S, 479W, 480G, 484E, 484G, 484L, 484Q, 484T, 484W, 489H, 489L, 489Q, 491W, 492K, 492Y, 498C, 498D, 501A, 501L, 501S, 504S/548F, 505V, 510G, 510S, 510T, 519D/720S, 521W, 524K, 525G, 525Q, 525R, 525R/533C, 525S, 525V, 529S, 531A, 532L, 533G, 533L, 533N, 533R, 533W, 535N, 535S, 540C, 540S, 540T, 546C, 546R, 547M, 550G, 550I, 550P, 550Y, 553L, 553S, 555A, 555L, 555T/572H, 555V, 557L, 558Q, 559L, 559R, 566G, 568C, 568K, 570S, 571Y, 572N, 572T, 573T, 574M, 575C, 575R, 576K, 576W, 577G, 577H, 577L, 577S, 577W, 583M, 583Q, 588F, 588G, 588V, 590F, 590I/652T, 592S, 597G, 597S, 597V, 600A, 600K, 600R, 600S, 601F, 601L, 612E, 612S, 612T, 612V, 615S, 615Y, 618C, 620L, 620M, 620T, 622G, 622L, 622S, 623G, 623L, 623V, 623W, 6251, 625T, 628M, 628T, 643G, 643L, 643M, 643V, 643Y, 654F, 654G, 654K, 655A, 655F, 655L, 655V, 660E, 660G, 660R, 660W, 664L, 664M, 675G, 675L, 675M, 675T, 677A, 682K, 685R, 685S, 690G, 690R, 690T, 690V, 690W, 691F/692H, 692L, 692S/766T, 692V, 697G, 697Q, 697S, 703F, 703G, 703K, 703P, 703R, 703V, 705F, 705G, 705M, 705V, 708R, 711S, 711V, 711W, 715Q, 715V, 720G, 726T, 728A, 728F, 728Q, 728R, 728S, 728V, 728W, 730R, 730V, 732E, 738G, 738M, 738W, 743A, 743G, 743Q, 743R, 743W, 746-, 752L, 752V, 753G, 753K, 753L, 753R, 756H, 756R, 756T, 766G, 766V, 769A, 769C, 769G, 769M, 769V, 770R, 779R, 793T, 800G, 800I, 800M, 800P, 802C, 802G, 802R, 803S, 805F, 805G, 805I, 805M, 805Q, 805T, 813G, 819A, 819L, 819R, 819S, 819W, 825G, 825S, 825Y, 827G, 827V, 827W, 829L, 829P, and 829W, or combinations thereof, wherein the amino acid positions are relative to the reference sequence of SEQ ID NO: 3796.

In some embodiments, the engineered DNA polymerase comprises one or more substitutions or substitution sets selected from M1H/H2M/A149G, V15M/E167V, P18H, P18H/A105F/R139D/P197K/K249E/L426R/P598K, P18H/A105F/R139D/P197K/L291Q/S299N/A423E/L426R/R448V/E529D/L547M/T725H, P18H/A105F/K249E/R284C/L302V/A423E/L426R/R501K/T673K/A684P/L798F, P18H/A105F/S321T/L547M, P18H/R139D/P197K/K249E/S299N/L302V/E433K/L547M/Q642E/S659G/Y695K/A744G, P18H/R139D/P197K/L426R/P598K, P18H/R139D/P197K/Q642E, P18H/R139D/K249E/A423E/E433K/R501K/L547M/T725H, P18H/P197K/K249E/L291Q/F484V, P18H/P197K/S321T/L426R/R448V/A451G/S659G/T673K/A744G, P18H/P197K/S321T/E433K/F484V, P18H/K249E, P18H/K249E/S299N/L302V/A423E/E529D/E533C/Q642E/S659G/T673K, P18H/K249E/L302V/A423E/F484V/L547M/Y695K, P18H/K249E/R448V/F484V/T673K, P18H/K249E/T673K, P18H/L291Q/L302V/A423E/A684P, P18H/L291Q/L302V/E433K, P18H/L291Q/L302V/A525R/L547M/S659G/A684P/Y695K, P18H/L291Q/L302V/A684P, P18H/L291Q/A423E/A525R/E529D, P18H/L291Q/S659G, P18H/L291Q/Y695K, P18H/S299N/L302V/A423E/L426R/A525R/E529D, P18H/S299N/A451G/A525R/E529D/Y695K, P18H/L302V/A423E/E529D, P18H/L302V/E433K/L547M/A684P, P18H/L302V/A525R/E529D/L547M, P18H/L302V/E529D/S659G/Y695K, P18H/S321T/A423E, P18H/S321T/F484V/L547M/T725H, P18H/A423E, P18H/A423E/E529D/Y695K, P18H/E433K, P18H/E433K/T673K/T725H, P18H/F484V, P18H/P598K/T725H, Y32G, Y32L, Y32S, L38M/A463G, K64G, A105F/R139D/L291Q/L302V/A423E/F484V/L547M/T725H, A105F/R139D/R448V/R501K/A525R/E529D/Q642E/S659G, A105F/P197K/K249E/A423E/L426R/L547M/Q642E/T673K, A105F/S321T/E433K/A744G, A105F/S321T/R501K, A137L, R139D/K249E/L291Q/S321T/F484V/Y695K/T725H, R139D/K249E/L291Q/A451G/A744G, R139D/K249E/L426R/A451G/R501K/T673K/A744G, A149G, A149P, A149V, R160G, R160L, L164V/T172F, E167A, E167G, T172H, T172V, T172W, P197K/K249E/W327L/A423E/T673K/E690G, P197K/L302V/P391S/L547M/P598K, P197K/A525R/E529D/S659G/A744G, P197K/L547M, P197K/A684P/T725H, N200S/A577G, L245I/A423L, D247Y/A423E/R448V, K249E/S321T/T673K/L798F, K249E/A423E/L426R/T673K, K249E/R448V/A451G/L547M/S659G/T725H, K249E/T725H/A744G/L798F, E253C, E253I, E253M, R256F, R256S, R256S/A455F, R256V, R271L, F281V, L291Q/L302V/A423E, L291Q/L302V/E529D, L291Q/S321T/A423E/T725H, L291Q/A423E/A525R, L291Q/A423E/E529D/E533C/P598K/Q642E/T725H/L798F, L291Q/F484V/P598K/Q642E/T673K/A744G/L798F, L291Q/A525R, P300G, L302G, L302M, L302S, L302V/D364N/L376F/M383K/A386G/T408A/A451G/F484V/P598K, L302V/A423E/E433K/E529D/Y695K, L302V/A423E/A525R/E529D, L302V/A423E/L547M/A684P/Y695K, L302V/A684P/Y695K, P311C, P311G, P311H, P311K, A314L, A314M, L316N, S321T/R448V/E529D/T673K, P323W, L330M, L330T, L330V, A334T/G431C, P347L, P347R, H348A, R352S/K363L, G353I, G353T, L354G, L354M, R358E, R358L, L361C, L361R, L361W, K363L, K363T, K363V, V368A, V368L, V368R, V368W, E372K, E372L, E372R, E372S, E372W, L376I, L376K, L376W, L376W/Q699L, M383I, M383R, M383S, L389G, L389R, L389S, S392G, S392R, S392T, S392V, T394D, A411E, R414S, H422A, H422G, H422L, H422Q, H422R, A423E/N424K/L547M, A423E/E433K/F484V/E529D/E533C/Q642E, A423E/E433K/A487V/A525R/L547M, A423E/F484V/T673K, A423E/E529D/A684P, A423E/L547M/P598K/Q642E, Q430R, G431D, G431Q, G431S, G431T, G431V, E433K/A525R/E529D/S659G/A684P/Y695K, L436F/N743K, R448V/A451G/E529D/T725H, A451G/P598K/T725H, A455E, A455G, A455L, A455P/T518I, A455Q, A455S, A455V, A455W, V462A, V462G, V462L, V462Q, V462R, V462S, V462W, A463T, Y464H, Y464K, Y464V, L468A, L468G, L468T, L468W, S469R, V472E, V472G, V472L, V472S, V472T, A474E, A474L/P821S, A474R, A474S, M476S, L479W, E480G, F484E, F484G, F484L, F484Q, F484T, F484W, R489H, R489L, R489Q, F491W, R492K, R492Y, Q498C, Q498D, R501A, R501L, R501S, F504S/A548F, D505V, P510G, P510S, P510T, G519D/L720S, R521W, S524K, A525G, A525Q, A525R, A525R/E533C, A525S, A525V, E529S, L531A, R532L, E533G, E533L, E533N, E533R, E533W, H535N, H535S, K540C, K540S, K540T, E546C, E546R, L547M, L550G, L550I, L550P, L550Y, T553L, T553S, 1555A, 1555L, 1555T/R572H, 1555V, R557L, L558Q, P559L, P559R, T566G, R568C, R568K, H570S, T571Y, R572N, R572T, F573T, N574M, Q575C, Q575R, T576K, T576W, A577G, A577H, A577L, A577S, A577W, L583M, L583Q, P588F, P588G, P588V, L590F, L590I/A652T, N592S, T597G, T597S, T597V, G600A, G600K, G600R, G600S, Q601F, Q601L, G612E, G612S, G612T, G612V, L615S, L615Y, L618C, Y620L, Y620M, Y620T, Q622G, Q622L, Q622S, 1623G, I623L, 1623V, 1623W, L625I, L625T, L628M, L628T, E643G, E643L, E643M, E643V, E643Y, W654F, W654G, W654K, M655A, M655F, M655L, M655V, A660E, A660G, A660R, A660W, D664L, D664M, N675G, N675L, N675M, N675T, G677A, M682K, F685R, F685S, E690G, E690R, E690T, E690V, E690W, L691F/G692H, G692L, G692S/A766T, G692V, E697G, E697Q, E697S, Q703F, Q703G, Q703K, Q703P, Q703R, Q703V, Y705F, Y705G, Y705M, Y705V, S708R, K711S, K711V, K711W, W715Q, W715V, L720G, R726T, Y728A, Y728F, Y728Q, Y728R, Y728S, Y728V, Y728W, E730R, E730V, L732E, Y738G, Y738M, Y738W, N743A, N743G, N743Q, N743R, N743W, V746-, A752L, A752V, A753G, A753K, A753L, A753R, M756H, M756R, M756T, A766G, A766V, L769A, L769C, L769G, L769M, L769V, M770R, P779R, H793T, A800G, A800I, A800M, A800P, K802C, K802G, K802R, E803S, A805F, A805G, A805I, A805M, A805Q, A805T, K813G, V819A, V819L, V819R, V819S, V819W, P825G, P825S, P825Y, E827G, E827V, E827W, E829L, E829P, and E829W, or combinations thereof, wherein the amino acid positions are relative to the reference sequence of SEQ ID NO: 3796.

The present invention also provides engineered DNA polymerases or a functional fragment thereof, comprising a polypeptide sequence having at least 75% or more sequence identity to a reference sequence comprising residues 12 to 841 of SEQ ID NO: 4182, or to a reference sequence of SEQ ID NO: 4182, wherein the polypeptide sequence comprises one or more substitutions relative to the reference sequence comprising residues 12 to 841 of SEQ ID NO: 4182, or to the reference sequence of SEQ ID NO: 4182. In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 4182 or to the reference sequence of SEQ ID NO: 4182.

In some embodiments, the engineered DNA polymerase comprises one or more substitutions or substitution sets at positions selected from 18/149/300/423/424/743/769, 18/256/423/424/455/673, 18/372/424/533/546/600/673/769, 18/424, 149/256/300/302/424/484/769, 149/256/300/372/424/546, 149/424, 256/300/363/423/424/546/673/690, 424, 424/533, and 424/723, or combinations thereof, wherein the amino acid positions are relative to the reference sequence of SEQ ID NO: 4182.

In some embodiments, the engineered DNA polymerase comprises one or more substitutions or substitution sets selected from 18H/149G/300G/423A/424N/743A/769C, 18H/256S/423A/424N/455S/673K, 18H/372S/424N/533W/546C/600A/673K/769C, 18H/424N, 149G/256S/300G/302G/424N/484V/769C, 149G/256S/300G/372S/424N/546C, 149G/424N, 256S/300G/363L/423A/424N/546C/673K/690V, 424N, 424N/533W, and 424N/723D, or combinations thereof, wherein the amino acid positions are relative to the reference sequence of SEQ ID NO: 4182.

In some embodiments, the engineered DNA polymerase comprises one or more substitutions or substitution sets selected from P18H/A149G/P300G/E423A/K424N/N743A/L769C, P18H/R256S/E423A/K424N/A455S/T673K, P18H/E372S/K424N/E533W/E546C/G600A/T673K/L769C, P18H/K424N, A149G/R256S/P300G/L302G/K424N/F484V/L769C, A149G/R256S/P300G/E372S/K424N/E546C, A149G/K424N, R256S/P300G/K363L/E423A/K424N/E546C/T673K/E690V, K424N, K424N/E533W, and K424N/G723D, or combinations thereof, wherein the amino acid positions are relative to the reference sequence of SEQ ID NO: 4182.

The present invention also provides engineered DNA polymerases or a functional fragment thereof, comprising a polypeptide sequence having at least 75% or more sequence identity to a reference sequence comprising residues 12 to 841 of SEQ ID NO: 5094, or to a reference sequence of SEQ ID NO: 5094, wherein the polypeptide sequence comprises one or more substitutions relative to the reference sequence comprising residues 12 to 841 of SEQ ID NO: 5094, or to the reference sequence of SEQ ID NO: 5094. In some embodiments, the engineered DNA polymerases comprise a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5094 or to the reference sequence of SEQ ID NO: 5094.

In some embodiments, the engineered DNA polymerase comprises one or more substitutions or substitution sets at positions selected from 9/689, 9/691, 21/695/700, 45, 45/268, 45/268/302/423, 45/268/302/467/621/748/749, 45/268/302/469, 45/268/302/469/547/557/721/749, 45/268/302/475/489, 45/268/302/547/557/621/748/749, 45/268/423/467/748, 45/268/423/469/721, 45/268/423/475/547/721/749, 45/268/469, 45/268/475, 45/268/475/489, 45/268/475/621, 45/268/489/621/749, 45/268/621/721, 45/302, 45/302/423/469/475, 45/302/423/469/621, 45/302/423/469/621/721/748/749, 45/302/423/547/621/721, 45/302/469/547/721, 45/302/475/621/748/749, 45/302/621, 45/423, 45/423/467, 45/423/467/469/547/721/749, 45/423/467/469/749, 45/423/467/475/547/621/721/749, 45/423/467/621, 45/423/469/475, 45/423/469/475/621/721/749, 45/423/469/489/621, 45/423/475/547/721, 45/423/475/557/621/749, 45/423/489, 45/423/489/557, 45/467/469/557, 45/467/621, 45/467/748, 45/469/721, 45/469/749, 45/475/489/547/749, 45/547/749, 45/557, 45/721, 83/700/744, 95/268/302/423/489/547/557/721/748/749, 116, 228/708, 268, 268/302/423/557/721, 268/302/721, 268/423, 268/423/467/547, 268/423/469, 268/423/469/475, 268/467, 268/467/469/475, 268/467/748, 268/475/547, 268/475/557/721/749, 268/489/547, 296/433, 302, 302/423/467/621/748, 302/423/469/489/721/748/749, 302/423/475/489/547/621, 302/423/547/721, 302/423/557, 302/467/469/547/557, 302/467/489/748/749, 302/467/621, 302/467/721/748, 302/469, 302/469/489/621/721/748, 302/489, 302/489/547/621/699/749, 302/557/721, 302/621, 302/721, 302/749, 306, 306/459, 314/538, 321, 322, 329/359/611/700/744, 332, 335, 339, 341/532, 344, 345, 351, 354/359/408/598, 359, 359/408/412, 359/408/412/611/614/744, 359/408/412/611/708/744, 359/408/412/611/744/789, 359/408/412/700/744, 359/408/412/744, 359/408/433/614/708, 359/408/598/700, 359/408/611/614/744, 359/408/695/700/744, 359/408/695/744, 359/412, 359/412/433/436/598/611/614, 359/412/433/611/614/708, 359/412/433/611/614/744/789, 359/412/436/598/611/695/744, 359/412/469/611/695/708/744, 359/412/598, 359/412/598/611/695/700/708/744, 359/412/598/611/744, 359/412/695/700/744, 359/412/695/708/744, 359/433/611/614/700, 359/433/611/695/700/789, 359/436/611/708/744, 359/469/611, 359/469/611/614, 359/469/611/700, 359/598/611/614/744, 359/598/614/695/708/744, 359/598/700/744, 359/598/744/789, 359/611/614/695/700/744, 359/611/614/744, 359/611/700/744, 359/614, 359/695/700/789, 359/700, 359/744, 359/789, 364, 373, 378, 385, 386, 387/630, 393, 397, 399, 404, 408, 408/412, 408/412/433/436/611/614/744, 408/412/433/598/611/700/708/744, 408/412/436/598/708, 408/412/436/611/614, 408/412/469/475/611/695/708/744, 408/412/598/611/614/708, 408/412/598/700/744, 408/412/611/695/708, 408/412/708/744, 408/433/700/708/744, 408/436/598/611/708/744, 408/436/695/708/744, 408/598/611, 408/598/611/614/700, 408/598/695/700, 408/611/614/695/708, 408/611/614/708/744, 408/611/614/744, 408/611/744, 408/700, 408/744, 409, 412, 412/433/598/611/744, 412/433/611/744, 412/436/611/614/744/789, 412/436/744, 412/469/611/695/744, 412/469/611/700/744, 412/598/611/744, 412/611/614, 412/611/614/708/744/789, 412/611/789, 412/614/708, 412/689, 412/695/789, 412/708/744, 413, 417, 423, 423/467/469, 423/467/489/621/721, 423/467/547/557/621/699/721, 423/467/621/721/749, 423/467/721, 423/469, 423/469/475/489/721/748/749, 423/469/489/557/621/721/749, 423/469/547/557/621/749, 423/469/547/557/749, 423/469/621, 423/469/721, 423/489, 423/489/621, 423/598/700/708/744, 423/721, 423/748, 424, 425, 426, 433, 433/436/598/700/744, 433/436/598/744, 433/436/611/614, 433/469/611, 433/611/695/700/744, 433/744/789, 436, 436/611/614, 436/611/614/695, 436/700/744, 436/708/744, 439, 443, 446, 448, 449, 467/469, 467/489/547/621/748, 467/547/721, 467/621, 467/721, 467/721/748, 467/748, 467/749, 469, 469/471, 469/473, 469/475/621/721/748, 469/475/749, 469/614/695, 469/614/695/744, 469/621, 469/621/748, 469/744, 469/748, 475, 483, 485/791, 487, 489, 506, 507, 509, 511, 514, 517, 520, 532, 533/542, 538, 539, 539/571, 542, 546/699, 547, 547/621/721/748/749, 549, 554, 557, 557/621, 580, 584, 587, 596, 598, 598/611, 598/611/614, 598/611/708, 598/614/700/744, 598/708, 602, 602/724, 611, 611/614, 611/614/708/744/789, 611/744, 614, 614/700/708/744/789, 614/744, 614/789, 617, 621, 621/639/748/749, 621/745, 621/748, 621/749, 630, 652, 689, 691, 695, 695/708/744, 695/744, 699, 700, 700/744, 700/767, 708, 714, 721, 721/749, 723, 725, 741, 744, 745, 748, 748/749, 767, 777, 780, 785, 785/809, 787, 789, 791, 795, 798, 799, 811, 817, 821, 836, 838, and 839, or combinations thereof, wherein the amino acid positions are relative to the reference sequence of SEQ ID NO: 5094.

In some embodiments, the engineered DNA polymerase comprises one or more substitutions or substitution sets selected from 9D/691S, 9V/689G, 21S/695L/700G, 45R, 45R/268R, 45R/268R/302P/423A, 45R/268R/302P/467A/621S/748S/749V, 45R/268R/302P/469H, 45R/268R/302P/469H/547L/557P/721E/749V, 45R/268R/302P/475E/489H, 45R/268R/302P/547L/557P/621S/748S/749V, 45R/268R/423A/467A/748S, 45R/268R/423A/469H/721E, 45R/268R/423A/475E/547L/721E/749V, 45R/268R/469H, 45R/268R/475E, 45R/268R/475E/489H, 45R/268R/475E/621S, 45R/268R/489H/621S/749V, 45R/268R/621S/721E, 45R/302P, 45R/302P/423A/469H/475E, 45R/302P/423A/469H/621S, 45R/302P/423A/469H/621S/721E/748S/749V, 45R/302P/423A/547L/621S/721E, 45R/302P/469H/547L/721E, 45R/302P/475E/621S/748S/749V, 45R/302P/621S, 45R/423A, 45R/423A/467A, 45R/423A/467A/469H/547L/721E/749V, 45R/423A/467A/469H/749V, 45R/423A/467A/475E/547L/621S/721E/749V, 45R/423A/467A/621S, 45R/423A/469H/475E, 45R/423A/469H/475E/621S/721E/749V, 45R/423A/469H/489H/621S, 45R/423A/475E/547L/721E, 45R/423A/475E/557P/621S/749V, 45R/423A/489H, 45R/423A/489H/557P, 45R/467A/469H/557P, 45R/467A/621S, 45R/467A/748S, 45R/469H/721E, 45R/469H/749V, 45R/475E/489H/547L/749V, 45R/547L/749V, 45R/557P, 45R/721E, 83N/700G/744G, 95S/268R/302P/423A/489H/547L/557P/721E/748S/749V, 116P, 228D/708E, 268R, 268R/302P/423A/557P/721E, 268R/302P/721E, 268R/423A, 268R/423A/467A/547L, 268R/423A/469H, 268R/423A/469H/475E, 268R/467A, 268R/467A/469H/475E, 268R/467A/748S, 268R/475E/547L, 268R/475E/557P/721E/749V, 268R/489H/547L, 296H/433L, 302P, 302P/423A/467A/621S/748S, 302P/423A/469H/489H/721E/748S/749V, 302P/423A/475E/489H/547L/621S, 302P/423A/547L/721E, 302P/423A/557P, 302P/467A/469H/547L/557P, 302P/467A/489H/748S/749V, 302P/467A/621S, 302P/467A/721E/748S, 302P/469H, 302P/469H/489H/621S/721E/748S, 302P/489H, 302P/489H/547L/621S/699A/749V, 302P/557P/721E, 302P/621S, 302P/721E, 302P/749V, 306E, 306L, 306M, 306N, 306P, 306R, 306S, 306T, 306T/459E, 306V, 314S/538L, 321A, 321I, 321W, 322A, 322C, 322D, 322G, 322L, 322M, 322P, 322S, 322T, 329K/359T/611P/700G/744S, 332C, 332D, 332I, 332L, 332M, 332Q, 332R, 332T, 332V, 335C, 335G, 335S, 339A, 339H, 339L, 339P, 339S, 339V, 341M/532L, 344G, 344P, 344R, 344S, 344V, 345G, 345K, 345L, 351M, 351V, 354I/359T/408W/598F, 359T, 359T/408W/412G, 359T/408W/412G/611K/614A/744S, 359T/408W/412G/611K/744S/789D, 359T/408W/412G/611P/708G/744S, 359T/408W/412G/700G/744G, 359T/408W/412G/744S, 359T/408W/433A/614A/708G, 359T/408W/598F/700G, 359T/408W/611K/614A/744G, 359T/408W/695L/700G/744G, 359T/408W/695L/744G, 359T/412G, 359T/412G/433A/436R/598F/611P/614A, 359T/412G/433A/611P/614A/708G, 359T/412G/433A/611P/614A/744S/789D, 359T/412G/436R/598F/611P/695L/744G, 359T/412G/469L/611P/695L/708G/744S, 359T/412G/598F, 359T/412G/598F/611K/744S, 359T/412G/598F/611P/695L/700G/708G/744S, 359T/412G/695L/700G/744G, 359T/412G/695L/708G/744G, 359T/433A/611P/614A/700G, 359T/433A/611P/695L/700G/789D, 359T/436R/611P/708G/744G, 359T/469L/611K, 359T/469L/611P/614A, 359T/469L/611P/700G, 359T/598F/611P/614A/744S, 359T/598F/614A/695L/708G/744S, 359T/598F/700G/744G, 359T/598F/744S/789D, 359T/611K/614A/744G, 359T/611P/614A/695L/700G/744G, 359T/611P/700G/744S, 359T/614A, 359T/695L/700G/789D, 359T/700G, 359T/744S, 359T/789D, 364G, 364P, 364R, 364T, 364V, 373F, 373K, 373P, 373S, 373V, 378C, 378F, 378G, 378L, 378R, 378Y, 385A, 385E, 385G, 385P, 385R, 385V, 386C, 386G, 386L, 386Q, 386S, 386V, 386W, 387C/630E, 393A, 393E, 393G, 393P, 393R, 393Y, 397A, 397D, 397G, 397L, 397N, 397P, 397R, 397V, 399E, 399G, 399L, 399P, 399Q, 399R, 399S, 399W, 404F, 404I, 404K, 404L, 404P, 404S, 404T, 404W, 408A, 408L, 408N, 408P, 408Q, 408R, 408W, 408W/412G, 408W/412G/433A/436R/611P/614A/744G, 408W/412G/433A/598F/611P/700G/708G/744S, 408W/412G/436R/598F/708G, 408W/412G/436R/611P/614A, 408W/412G/469L/475Q/611P/695L/708G/744S, 408W/412G/598F/611P/614A/708G, 408W/412G/598F/700G/744S, 408W/412G/611P/695L/708G, 408W/412G/708G/744G, 408W/433A/700G/708G/744S, 408W/436R/598F/611P/708G/744S, 408W/436R/695L/708G/744G, 408W/598F/611P, 408W/598F/611P/614A/700G, 408W/598F/695L/700G, 408W/611K/614A/744G, 408W/611P/614A/695L/708G, 408W/611P/614A/708G/744G, 408W/611P/744S, 408W/700G, 408W/744G, 408W/744S, 409G, 412F, 412G, 412G/433A/598F/611P/744G, 412G/433A/611P/744S, 412G/436R/611K/614A/744S/789D, 412G/436R/744G, 412G/469L/611P/695L/744G, 412G/469L/611P/700G/744S, 412G/598F/611P/744S, 412G/611K/614A, 412G/611P/614A/708G/744S/789D, 412G/611P/789D, 412G/614A/708G, 412G/695L/789D, 412G/708G/744G, 412K, 412L, 412P, 412Q, 412R, 412T/689V, 413E, 413G, 413H, 413L, 413R, 413S, 413T, 413W, 417A, 417G, 417Q, 417S, 423A, 423A/467A/469H, 423A/467A/489H/621S/721E, 423A/467A/547L/557P/621S/699A/721E, 423A/467A/621S/721E/749V, 423A/467A/721E, 423A/469H, 423A/469H/475E/489H/721E/748S/749V, 423A/469H/489H/557P/621S/721E/749V, 423A/469H/547L/557P/621S/749V, 423A/469H/547L/557P/749V, 423A/469H/621S, 423A/469H/721E, 423A/489H, 423A/489H/621S, 423A/721E, 423A/748S, 423G/598F/700G/708G/744S, 424E, 424G, 424L, 424T, 425C, 425S, 425T, 425Y, 426G, 426R, 426S, 426T, 426V, 433A, 433A/436R/598F/700G/744G, 433A/436R/598F/744G, 433A/436R/611P/614A, 433A/469L/611P, 433A/611P/695L/700G/744G, 433A/744S/789D, 433G, 433Q, 433R, 433S, 433W, 436A, 436G, 436Q, 436R, 436R/611K/614A, 436R/611P/614A/695L, 436R/700G/744G, 436R/708G/744G, 436S, 436T, 436V, 436W, 439A, 439E, 439L, 439S, 439W, 443C, 443T, 446S, 448E, 448L, 448W, 449G, 449W, 467A/469H, 467A/489H/547L/621S/748S, 467A/547L/721E, 467A/621S, 467A/721E, 467A/721E/748S, 467A/748S, 467A/749V, 467C/469H, 467H/469H, 467M/469H, 467R/469H, 467T/469H, 467V/469H, 467W/469H, 469E, 469H, 469H/471A, 469H/471F, 469H/471H, 469H/471L, 469H/471M, 469H/471Q, 469H/471R, 469H/473F, 469H/473W, 469H/475E/621S/721E/748S, 469H/475E/749V, 469H/621S, 469H/621S/748S, 469H/748S, 469L, 469L/614A/695L, 469L/614A/695L/744S, 469L/744S, 469R, 469W, 469Y, 475E, 483A, 483C, 483G, 483L, 483Q, 483S, 483T, 485H/791S, 487G, 487L, 487R, 487S, 487V, 487Y, 489H, 506G, 506H, 506M, 506R, 506V, 507G, 507K, 507Q, 509F, 509M, 509V, 511A, 511D, 511L, 511N, 511R, 511V, 511Y, 514G, 514M, 514T, 514W, 517A, 517L, 517M, 517R, 517S, 517V, 517W, 520S, 520V, 532T, 532W, 532Y, 533K/542S, 538A, 538G, 538L, 538P, 538S, 539A, 539G, 539G/571A, 539L, 542A, 542G, 542K, 542P, 542R, 542S, 542W, 546G/699A, 547L, 547L/621S/721E/748S/749V, 549A, 549H, 549L, 549M, 549N, 549P, 549S, 549T, 549V, 549Y, 554S, 557P, 557P/621S, 580K, 584A, 584Q, 584V, 587G, 587M, 587S, 587T, 587V, 587W, 596W, 598A, 598C, 598F, 598F/611P, 598F/611P/614A, 598F/611P/708G, 598F/614A/700G/744G, 598F/708G, 598G, 598L, 598R, 598S, 598V, 602A, 602G, 602S, 602S/724H, 602V, 602W, 611C, 611I, 611K, 611L, 611P, 611P/614A, 611P/614A/708G/744S/789D, 611P/744S, 611R, 611S, 611T, 611V, 611W, 614A, 614A/700G/708G/744S/789D, 614A/744G, 614A/789D, 614G, 614P, 614T, 614W, 617C, 617D, 617G, 617H, 617L, 617R, 617S, 617V, 621H/745D, 621S, 621S/639H/748S/749V, 621S/748S, 621S/749V, 630A, 630C, 630F, 630G, 630M, 630S, 630T, 630W, 652G, 652S, 652V, 689G, 689K, 689L, 689M, 689P, 689R, 689S, 689W, 691A, 691G, 691K, 691Q, 691R, 691S, 691T, 691W, 695A, 695E, 695L, 695L/708G/744G, 695L/744G, 695P, 695R, 695S, 695T, 699A, 700E, 700F, 700G, 700G/744G, 700G/767T, 700L, 700R, 700V, 700W, 700Y, 708A, 708G, 708R, 708T, 714G, 714L, 714P, 714R, 714S, 714V, 714Y, 721E, 721E/749V, 723A, 723Q, 723T, 723V, 725C, 725E, 725L, 725P, 725S, 725V, 725W, 741A, 741E, 741H, 741K, 741L, 741P, 744F, 744G, 744H, 744P, 744R, 744S, 744T, 744W, 745C, 745D, 745G, 745P, 745Q, 745V, 745W, 748D, 748R, 748S, 748S/749V, 748V, 767G, 767T, 777A, 777C, 777Q, 777T, 777V, 780A, 780R, 785Q, 785R, 785R/809V, 787E, 787G, 787K, 787P, 787W, 789A, 789D, 789E, 789G, 789P, 789Q, 789S, 789T, 789V, 789Y, 791A, 791G, 791H, 791L, 791R, 791T, 791V, 795D, 795S, 798T, 799M, 799Q, 811G, 811R, 811S, 811W, 817A, 817D, 817F, 817H, 817K, 817N, 817Q, 817S, 817V, 817W, 821D, 821G, 821K, 821L, 821Q, 821S, 836L, 838R, 838T, 838V, 839E, 839L, 839P, 839R, 839T, and 839V, or combinations thereof, wherein the amino acid positions are relative to the reference sequence of SEQ ID NO: 5094.

In some embodiments, the engineered DNA polymerase comprises one or more substitutions or substitution sets selected from G9D/L691S, G9V/Q689G, R21S/Y695L/A700G, L45R, L45R/G268R, L45R/G268R/L302P/E423A, L45R/G268R/L302P/N467A/R621S/G748S/K749V, L45R/G268R/L302P/S469H, L45R/G268R/L302P/S469H/M547L/R557P/R721E/K749V, L45R/G268R/L302P/H475E/R489H, L45R/G268R/L302P/M547L/R557P/R621S/G748S/K749V, L45R/G268R/E423A/N467A/G748S, L45R/G268R/E423A/S469H/R721E, L45R/G268R/E423A/H475E/M547L/R721E/K749V, L45R/G268R/S469H, L45R/G268R/H475E, L45R/G268R/H475E/R489H, L45R/G268R/H475E/R621S, L45R/G268R/R489H/R621S/K749V, L45R/G268R/R621S/R721E, L45R/L302P, L45R/L302P/E423A/S469H/H475E, L45R/L302P/E423A/S469H/R621S, L45R/L302P/E423A/S469H/R621S/R721E/G748S/K749V, L45R/L302P/E423A/M547L/R621S/R721E, L45R/L302P/S469H/M547L/R721E, L45R/L302P/H475E/R621S/G748S/K749V, L45R/L302P/R621S, L45R/E423A, L45R/E423A/N467A, L45R/E423A/N467A/S469H/M547L/R721E/K749V, L45R/E423A/N467A/S469H/K749V, L45R/E423A/N467A/H475E/M547L/R621S/R721E/K749V, L45R/E423A/N467A/R621S, L45R/E423A/S469H/H475E, L45R/E423A/S469H/H475E/R621S/R721E/K749V, L45R/E423A/S469H/R489H/R621S, L45R/E423A/H475E/M547L/R721E, L45R/E423A/H475E/R557P/R621S/K749V, L45R/E423A/R489H, L45R/E423A/R489H/R557P, L45R/N467A/S469H/R557P, L45R/N467A/R621S, L45R/N467A/G748S, L45R/S469H/R721E, L45R/S469H/K749V, L45R/H475E/R489H/M547L/K749V, L45R/M547L/K749V, L45R/R557P, L45R/R721E, H83N/A700G/A744G, P95S/G268R/L302P/E423A/R489H/M547L/R557P/R721E/G748S/K749V, L116P, N228D/S708E, G268R, G268R/L302P/E423A/R557P/R721E, G268R/L302P/R721E, G268R/E423A, G268R/E423A/N467A/M547L, G268R/E423A/S469H, G268R/E423A/S469H/H475E, G268R/N467A, G268R/N467A/S469H/H475E, G268R/N467A/G748S, G268R/H475E/M547L, G268R/H475E/R557P/R721E/K749V, G268R/R489H/M547L, L296H/E433L, L302P, L302P/E423A/N467A/R621S/G748S, L302P/E423A/S469H/R489H/R721E/G748S/K749V, L302P/E423A/H475E/R489H/M547L/R621S, L302P/E423A/M547L/R721E, L302P/E423A/R557P, L302P/N467A/S469H/M547L/R557P, L302P/N467A/R489H/G748S/K749V, L302P/N467A/R621S, L302P/N467A/R721E/G748S, L302P/S469H, L302P/S469H/R489H/R621S/R721E/G748S, L302P/R489H, L302P/R489H/M547L/R621S/Q699A/K749V, L302P/R557P/R721E, L302P/R621S, L302P/R721E, L302P/K749V, A306E, A306L, A306M, A306N, A306P, A306R, A306S, A306T, A306T/K459E, A306V, A314S/V538L, S321A, S321I, S321W, R322A, R322C, R322D, R322G, R322L, R322M, R322P, R322S, R322T, E329K/G359T/E611P/A700G/A744S, A332C, A332D, A332I, A332L, A332M, A332Q, A332R, A332T, A332V, A335C, A335G, A335S, G339A, G339H, G339L, G339P, G339S, G339V, V341M/R532L, A344G, A344P, A344R, A344S, A344V, E345G, E345K, E345L, L351M, L351V, L354I/G359T/T408W/P598F, G359T, G359T/T408W/A412G, G359T/T408W/A412G/E611K/R614A/A744S, G359T/T408W/A412G/E611K/A744S/L789D, G359T/T408W/A412G/E611P/S708G/A744S, G359T/T408W/A412G/A700G/A744G, G359T/T408W/A412G/A744S, G359T/T408W/E433A/R614A/S708G, G359T/T408W/P598F/A700G, G359T/T408W/E611K/R614A/A744G, G359T/T408W/Y695L/A700G/A744G, G359T/T408W/Y695L/A744G, G359T/A412G, G359T/A412G/E433A/L436R/P598F/E611P/R614A, G359T/A412G/E433A/E611P/R614A/S708G, G359T/A412G/E433A/E611P/R614A/A744S/L789D, G359T/A412G/L436R/P598F/E611P/Y695L/A744G, G359T/A412G/S469L/E611P/Y695L/S708G/A744S, G359T/A412G/P598F, G359T/A412G/P598F/E611K/A744S, G359T/A412G/P598F/E611P/Y695L/A700G/S708G/A744S, G359T/A412G/Y695L/A700G/A744G, G359T/A412G/Y695L/S708G/A744G, G359T/E433A/E611P/R614A/A700G, G359T/E433A/E611P/Y695L/A700G/L789D, G359T/L436R/E611P/S708G/A744G, G359T/S469L/E611K, G359T/S469L/E611P/R614A, G359T/S469L/E611P/A700G, G359T/P598F/E611P/R614A/A744S, G359T/P598F/R614A/Y695L/S708G/A744S, G359T/P598F/A700G/A744G, G359T/P598F/A744S/L789D, G359T/E611K/R614A/A744G, G359T/E611P/R614A/Y695L/A700G/A744G, G359T/E611P/A700G/A744S, G359T/R614A, G359T/Y695L/A700G/L789D, G359T/A700G, G359T/A744S, G359T/L789D, D364G, D364P, D364R, D364T, D364V, G373F, G373K, G373P, G373S, G373V, P378C, P378F, P378G, P378L, P378R, P378Y, L385A, L385E, L385G, L385P, L385R, L385V, A386C, A386G, A386L, A386Q, A386S, A386V, A386W, Y387C/H630E, N393A, N393E, N393G, N393P, N393R, N393Y, E397A, E397D, E397G, E397L, E397N, E397P, E397R, E397V, V399E, V399G, V399L, V399P, V399Q, V399R, V399S, V399W, G404F, G404I, G404K, G404L, G404P, G404S, G404T, G404W, T408A, T408L, T408N, T408P, T408Q, T408R, T408W, T408W/A412G, T408W/A412G/E433A/L436R/E611P/R614A/A744G, T408W/A412G/E433A/P598F/E611P/A700G/S708G/A744S, T408W/A412G/L436R/P598F/S708G, T408W/A412G/L436R/E611P/R614A, T408W/A412G/S469L/H475Q/E611P/Y695L/S708G/A744S, T408W/A412G/P598F/E611P/R614A/S708G, T408W/A412G/P598F/A700G/A744S, T408W/A412G/E611P/Y695L/S708G, T408W/A412G/S708G/A744G, T408W/E433A/A700G/S708G/A744S, T408W/L436R/P598F/E611P/S708G/A744S, T408W/L436R/Y695L/S708G/A744G, T408W/P598F/E611P, T408W/P598F/E611P/R614A/A700G, T408W/P598F/Y695L/A700G, T408W/E611K/R614A/A744G, T408W/E611P/R614A/Y695L/S708G, T408W/E611P/R614A/S708G/A744G, T408W/E611P/A744S, T408W/A700G, T408W/A744G, T408W/A744S, E409G, A412F, A412G, A412G/E433A/P598F/E611P/A744G, A412G/E433A/E611P/A744S, A412G/L436R/E611K/R614A/A744S/L789D, A412G/L436R/A744G, A412G/S469L/E611P/Y695L/A744G, A412G/S469L/E611P/A700G/A744S, A412G/P598F/E611P/A744S, A412G/E611K/R614A, A412G/E611P/R614A/S708G/A744S/L789D, A412G/E611P/L789D, A412G/R614A/S708G, A412G/Y695L/L789D, A412G/S708G/A744G, A412K, A412L, A412P, A412Q, A412R, A412T/Q689V, K413E, K413G, K413H, K413L, K413R, K413S, K413T, K413W, L417A, L417G, L417Q, L417S, E423A, E423A/N467A/S469H, E423A/N467A/R489H/R621S/R721E, E423A/N467A/M547L/R557P/R621S/Q699A/R721E, E423A/N467A/R621S/R721E/K749V, E423A/N467A/R721E, E423A/S469H, E423A/S469H/H475E/R489H/R721E/G748S/K749V, E423A/S469H/R489H/R557P/R621S/R721E/K749V, E423A/S469H/M547L/R557P/R621S/K749V, E423A/S469H/M547L/R557P/K749V, E423A/S469H/R621S, E423A/S469H/R721E, E423A/R489H, E423A/R489H/R621S, E423A/R721E, E423A/G748S, E423G/P598F/A700G/S708G/A744S, N424E, N424G, N424L, N424T, L425C, L425S, L425T, L425Y, L426G, L426R, L426S, L426T, L426V, E433A, E433A/L436R/P598F/A700G/A744G, E433A/L436R/P598F/A744G, E433A/L436R/E611P/R614A, E433A/S469L/E611P, E433A/E611P/Y695L/A700G/A744G, E433A/A744S/L789D, E433G, E433Q, E433R, E433S, E433W, L436A, L436G, L436Q, L436R, L436R/E611K/R614A, L436R/E611P/R614A/Y695L, L436R/A700G/A744G, L436R/S708G/A744G, L436S, L436T, L436V, L436W, Y439A, Y439E, Y439L, Y439S, Y439W, E443C, E443T, L446S, R448E, R448L, R448W, V449G, V449W, N467A/S469H, N467A/R489H/M547L/R621S/G748S, N467A/M547L/R721E, N467A/R621S, N467A/R721E, N467A/R721E/G748S, N467A/G748S, N467A/K749V, N467C/S469H, N467H/S469H, N467M/S469H, N467R/S469H, N467T/S469H, N467V/S469H, N467W/S469H, S469E, S469H, S469H/E471A, S469H/E471F, S469H/E471H, S469H/E471L, S469H/E471M, S469H/E471Q, S469H/E471R, S469H/A473F, S469H/A473W, S469H/H475E/R621S/R721E/G748S, S469H/H475E/K749V, S469H/R621S, S469H/R621S/G748S, S469H/G748S, S469L, S469L/R614A/Y695L, S469L/R614A/Y695L/A744S, S469L/A744S, S469R, S469W, S469Y, H475E, V483A, V483C, V483G, V483L, V483Q, V483S, V483T, R485H/Q791S, A487G, A487L, A487R, A487S, A487V, A487Y, R489H, E506G, E506H, E506M, E506R, E506V, L507G, L507K, L507Q, L509F, L509M, L509V, P511A, P511D, P511L, P511N, P511R, P511V, P511Y, K514G, K514M, K514T, K514W, K517A, K517L, K517M, K517R, K517S, K517V, K517W, K520S, K520V, R532T, R532W, R532Y, E533K/L542S, V538A, V538G, V538L, V538P, V538S, D539A, D539G, D539G/T571A, D539L, L542A, L542G, L542K, L542P, L542R, L542S, L542W, E546G/Q699A, M547L, M547L/R621S/R721E/G748S/K749V, K549A, K549H, K549L, K549M, K549N, K549P, K549S, K549T, K549V, K549Y, Y554S, R557P, R557P/R621S, T580K, S584A, S584Q, S584V, D587G, D587M, D587S, D587T, D587V, D587W, R596W, P598A, P598C, P598F, P598F/E611P, P598F/E611P/R614A, P598F/E611P/S708G, P598F/R614A/A700G/A744G, P598F/S708G, P598G, P598L, P598R, P598S, P598V, K602A, K602G, K602S, K602S/R724H, K602V, K602W, E611C, E611I, E611K, E611L, E611P, E611P/R614A, E611P/R614A/S708G/A744S/L789D, E611P/A744S, E611R, E611S, E611T, E611V, E611W, R614A, R614A/A700G/S708G/A744S/L789D, R614A/A744G, R614A/L789D, R614G, R614P, R614T, R614W, A617C, A617D, A617G, A617H, A617L, A617R, A617S, A617V, R621H/R745D, R621S, R621S/R639H/G748S/K749V, R621S/G748S, R621S/K749V, H630A, H630C, H630F, H630G, H630M, H630S, H630T, H630W, A652G, A652S, A652V, Q689G, Q689K, Q689L, Q689M, Q689P, Q689R, Q689S, Q689W, L691A, L691G, L691K, L691Q, L691R, L691S, L691T, L691W, Y695A, Y695E, Y695L, Y695L/S708G/A744G, Y695L/A744G, Y695P, Y695R, Y695S, Y695T, Q699A, A700E, A700F, A700G, A700G/A744G, A700G/A767T, A700L, A700R, A700V, A700W, A700Y, S708A, S708G, S708R, S708T, A714G, A714L, A714P, A714R, A714S, A714V, A714Y, R721E, R721E/K749V, G723A, G723Q, G723T, G723V, T725C, T725E, T725L, T725P, T725S, T725V, T725W, D741A, D741E, D741H, D741K, D741L, D741P, A744F, A744G, A744H, A744P, A744R, A744S, A744T, A744W, R745C, R745D, R745G, R745P, R745Q, R745V, R745W, G748D, G748R, G748S, G748S/K749V, G748V, A767G, A767T, L777A, L777C, L777Q, L777T, L777V, Q780A, Q780R, G785Q, G785R, G785R/A809V, R787E, R787G, R787K, R787P, R787W, L789A, L789D, L789E, L789G, L789P, L789Q, L789S, L789T, L789V, L789Y, Q791A, Q791G, Q791H, Q791L, Q791R, Q791T, Q791V, E795D, E795S, L798T, E799M, E799Q, L811G, L811R, L811S, L811W, E817A, E817D, E817F, E817H, E817K, E817N, E817Q, E817S, E817V, E817W, P821D, P821G, P821K, P821L, P821Q, P821S, W836L, A838R, A838T, A838V, A839E, A839L, A839P, A839R, A839T, and A839V, or combinations thereof, wherein the amino acid positions are relative to the reference sequence of SEQ ID NO: 5094.

In some embodiments, the engineered DNA polymerase comprises one or more substitutions or substitution sets at positions selected from 45/268/302/469, 45/268/475, 45/268/475/489, 45/268/621/721, 45/423/467/469/749, 45/721, 268, 268/475/547, 302, 302/489/547/621/699/749, 302/721, 302/749, 306, 359, 359/412/433/611/614/744/789, 359/436/611/708/744, 385, 408, 412, 412/598/611/744, 417, 423/467/721, 426, 433, 436, 436/708/744, 467/469, 469, 469/475/621/721/748, 469/475/749, 469/621/748, 469/748, 489, 511, 517, 547, 549, 557, 598, 598/611/708, 611, 614, 621/639/748/749, 630, 695, 699, 700, 708, 714, 721, 725, 741, 744, 748, 787, 789, 791, 795, and 817, or combinations thereof, wherein the amino acid positions are relative to the reference sequence of SEQ ID NO: 5094.

In some embodiments, the engineered DNA polymerase comprises one or more substitutions or substitution sets selected from 45R/268R/302P/469H, 45R/268R/475E, 45R/268R/475E/489H, 45R/268R/621S/721E, 45R/423A/467A/469H/749V, 45R/721E, 268R, 268R/475E/547L, 302P, 302P/489H/547L/621S/699A/749V, 302P/721E, 302P/749V, 306P, 359T, 359T/412G/433A/611P/614A/744S/789D, 359T/436R/611P/708G/744G, 385V, 408W, 412G, 412G/598F/611P/744S, 412P, 417G, 423A/467A/721E, 426R, 433A, 436R, 436R/708G/744G, 467A/469H, 469H/475E/621S/721E/748S, 469H/475E/749V, 469H/621S/748S, 469H/748S, 469L, 489H, 511R, 517R, 547L, 549L, 557P, 598F, 598F/611P/708G, 611K, 611P, 614A, 621S/639H/748S/749V, 630M, 695L, 695P, 699A, 700G, 700R, 708G, 714S, 721E, 725P, 741H, 741L, 744G, 744S, 748S, 787W, 789D, 791R, 795S, 817A, and 817K, or combinations thereof, wherein the amino acid positions are relative to the reference sequence of SEQ ID NO: 5094.

In some embodiments, the engineered DNA polymerase comprises one or more substitutions or substitution sets selected from L45R/G268R/L302P/S469H, L45R/G268R/H475E, L45R/G268R/H475E/R489H, L45R/G268R/R621S/R721E, L45R/E423A/N467A/S469H/K749V, L45R/R721E, G268R, G268R/H475E/M547L, L302P, L302P/R489H/M547L/R621S/Q699A/K749V, L302P/R721E, L302P/K749V, A306P, G359T, G359T/A412G/E433A/E611P/R614A/A744S/L789D, G359T/L436R/E611P/S708G/A744G, L385V, T408W, A412G, A412G/P598F/E611P/A744S, A412P, L417G, E423A/N467A/R721E, L426R, E433A, L436R, L436R/S708G/A744G, N467A/S469H, S469H/H475E/R621S/R721E/G748S, S469H/H475E/K749V, S469H/R621S/G748S, S469H/G748S, S469L, R489H, P511R, K517R, M547L, K549L, R557P, P598F, P598F/E611P/S708G, E611K, E611P, R614A, R621S/R639H/G748S/K749V, H630M, Y695L, Y695P, Q699A, A700G, A700R, S708G, A714S, R721E, T725P, D741H, D741L, A744G, A744S, G748S, R787W, L789D, Q791R, E795S, E817A, and E817K, or combinations thereof, wherein the amino acid positions are relative to the reference sequence of SEQ ID NO: 5094.

The present invention also provides engineered DNA polymerases or a functional fragment thereof, comprising a polypeptide sequence having at least 75% or more sequence identity to a reference sequence comprising residues 12 to 841 of SEQ ID NO: 5136, or to a reference sequence of SEQ ID NO: 5136, wherein the polypeptide sequence comprises one or more substitutions relative to the reference sequence comprising residues 12 to 841 of SEQ ID NO: 5136, or to the reference sequence of SEQ ID NO: 5136. In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5136 or to the reference sequence of SEQ ID NO: 5136.

In some embodiments, the engineered DNA polymerase comprises one or more substitutions or substitution sets at positions selected from 359/408/614, 359/412/436/598/611/614/695/700/708/744, 359/412/598/611/614/700/708/744, 359/412/611/708/744, 359/433/744, 359/436/598, 359/469/700/744, 359/598/611, 359/598/611/695/708, 359/598/695, 359/611/700/708, 359/744, 359/789, 408, 408/412/436/614/700/744, 408/412/469, 408/412/611, 408/412/744, 408/433/436/611/614/744, 408/433/436/695/708/744, 408/611, 412, 412/433, 412/611/614/708/744/789, 412/700/744, 433/436/598/611/614/708/744, 433/436/611/614/744, 433/436/611/744, 433/436/744, 433/614/700/744, 433/708, 436/598, 436/598/614/695/744, 436/611, 436/611/614, 436/611/614/695/700/744, 436/611/708, 436/744, 598/611/614/708/744, 598/614/695/700/708/744, 611, 611/614, 611/614/695/700/708/744, 611/708/744, 614/744, 614/789, 695, 695/700/744, 700, 700/744, and 744/789, or combinations thereof, wherein the amino acid positions are relative to the reference sequence of SEQ ID NO: 5136.

In some embodiments, the engineered DNA polymerase comprises one or more substitutions or substitution sets selected from 359T/408W/614R, 359T/412G/436R/598F/611K/614R/695L/700G/708G/744S, 359T/412G/598F/611P/614R/700G/708G/744S, 359T/412G/611K/708G/744G, 359T/433A/744G, 359T/436R/598F, 359T/469L/700G/744G, 359T/598F/611K, 359T/598F/611K/695L/708G, 359T/598F/695L, 359T/611K/700G/708G, 359T/744S, 359T/789D, 408W, 408W/412G/436R/614R/700G/744S, 408W/412G/469L, 408W/412G/611P, 408W/412G/744G, 408W/433A/436R/611P/614R/744G, 408W/433A/436R/695L/708G/744S, 408W/611P, 412G, 412G/433A, 412G/611K/614R/708G/744S/789D, 412G/700G/744S, 433A/436R/598F/611P/614R/708G/744S, 433A/436R/611P/614R/744S, 433A/436R/611P/744G, 433A/436R/744G, 433A/614R/700G/744S, 433A/708G, 436R/598F, 436R/598F/614R/695L/744G, 436R/611P, 436R/611P/614R, 436R/611P/614R/695L/700G/744S, 436R/611P/708G, 436R/744S, 598F/611P/614R/708G/744G, 598F/614R/695L/700G/708G/744S, 611P, 611P/614R, 611P/614R/695L/700G/708G/744S, 611P/708G/744S, 614R/744G, 614R/789D, 695L, 695L/700G/744G, 700G, 700G/744S, and 744S/789D, or combinations thereof, wherein the amino acid positions are relative to the reference sequence of SEQ ID NO: 5136.

In some embodiments, the engineered DNA polymerase comprises one or more substitutions or substitution sets selected from G359T/T408W/A614R, G359T/A412G/L436R/P598F/E611K/A614R/Y695L/A700G/S708G/A744S, G359T/A412G/P598F/E611P/A614R/A700G/S708G/A744S, G359T/A412G/E611K/S708G/A744G, G359T/E433A/A744G, G359T/L436R/P598F, G359T/S469L/A700G/A744G, G359T/P598F/E611K, G359T/P598F/E611K/Y695L/S708G, G359T/P598F/Y695L, G359T/E611K/A700G/S708G, G359T/A744S, G359T/L789D, T408W, T408W/A412G/L436R/A614R/A700G/A744S, T408W/A412G/S469L, T408W/A412G/E611P, T408W/A412G/A744G, T408W/E433A/L436R/E611P/A614R/A744G, T408W/E433A/L436R/Y695L/S708G/A744S, T408W/E611P, A412G, A412G/E433A, A412G/E611K/A614R/S708G/A744S/L789D, A412G/A700G/A744S, E433A/L436R/P598F/E611P/A614R/S708G/A744S, E433A/L436R/E611P/A614R/A744S, E433A/L436R/E611P/A744G, E433A/L436R/A744G, E433A/A614R/A700G/A744S, E433A/S708G, L436R/P598F, L436R/P598F/A614R/Y695L/A744G, L436R/E611P, L436R/E611P/A614R, L436R/E611P/A614R/Y695L/A700G/A744S, L436R/E611P/S708G, L436R/A744S, P598F/E611P/A614R/S708G/A744G, P598F/A614R/Y695L/A700G/S708G/A744S, E611P, E611P/A614R, E611P/A614R/Y695L/A700G/S708G/A744S, E611P/S708G/A744S, A614R/A744G, A614R/L789D, Y695L, Y695L/A700G/A744G, A700G, A700G/A744S, and A744S/L789D, or combinations thereof, wherein the amino acid positions are relative to the reference sequence of SEQ ID NO: 5136.

In some embodiments, the engineered DNA polymerase comprises one or more substitutions or substitution sets at positions selected from 359/598/695, 408/412/436/614/700/744, 408/611, 412, 433/614/700/744, 436/598, 436/611, 436/611/614/695/700/744, and 700, or combinations thereof, wherein the amino acid positions are relative to the reference sequence of SEQ ID NO: 5136.

In some embodiments, the engineered DNA polymerase comprises one or more substitutions or substitution sets selected from 359T/598F/695L, 408W/412G/436R/614R/700G/744S, 408W/611P, 412G, 433A/614R/700G/744S, 436R/598F, 436R/611P, 436R/611P/614R/695L/700G/744S, and 700G, or combinations thereof, wherein the amino acid positions are relative to the reference sequence of SEQ ID NO: 5136.

In some embodiments, the engineered DNA polymerase comprises one or more substitutions or substitution sets selected from G359T/P598F/Y695L, T408W/A412G/L436R/A614R/A700G/A744S, T408W/E611P, A412G, E433A/A614R/A700G/A744S, L436R/P598F, L436R/E611P, L436R/E611P/A614R/Y695L/A700G/A744S, and A700G, or combinations thereof, wherein the amino acid positions are relative to the reference sequence of SEQ ID NO: 5136.

The present invention further provides engineered DNA polymerases comprising polypeptide sequences having at least 75%, 80%, 85%, 86%, 87%, 88%, 89%, 90%, 91%, 92%, 93%, 94%, 95%, 96%, 97%, 98%, 99%, or more sequence identity to a reference sequence comprising residues 12 to 841 of SEQ ID NO: 178, 994, 1858, 2176, 2310, 2994, 3796, 4182, 5094, and/or 5136, or to a reference sequence of SEQ ID NO: 178, 994, 1858, 2176, 2310, 2994, 3796, 4182, 5094, and/or 5136.

In some embodiments, the engineered DNA polymerase comprises a polypeptide sequence having at least 75%, 80%, 85%, 86%, 87%, 88%, 89%, 90%, 91%, 92%, 93%, 94%, 95%, 96%, 97%, 98%, 99%, or more sequence identity to a reference sequence comprising residues 12 to 841 of SEQ ID NO: 2, 178, 994, 1858, 2176, 2310, 2994, 3796, 4182, 5094, and/or 5136, or to a reference sequence of SEQ ID NO: 2, 178, 994, 1858, 2176, 2310, 2994, 3796, 4182, 5094, and/or 5136, with the proviso that the polypeptide sequence does not include the sequence comprising residues 12 to 841 of SEQ ID NO:2.

In some embodiments, the engineered DNA polymerase comprises a polypeptide sequence comprising one or more amino acid substitutions relative to the reference sequence comprising residues 12 to 841 of SEQ ID NO: 178, or to the reference sequence of SEQ ID NO: 178, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 994, or to the reference sequence of SEQ ID NO: 994, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 1858, or to the reference sequence of SEQ ID NO: 1858, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 2176, or to the reference sequence of SEQ ID NO: 2176, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 2310, or to the reference sequence of SEQ ID NO: 2310, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 2994, or to the reference sequence of SEQ ID NO: 2994, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 3796, or to the reference sequence of SEQ ID NO: 3796, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 4182, or to the reference sequence of SEQ ID NO: 4182, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 5094, or to the reference sequence of SEQ ID NO: 5094, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 5136, or to the reference sequence of SEQ ID NO: 5136, with the proviso that the polypeptide sequence does not include the sequence comprising residues 12 to 841 of SEQ ID NO: 2.

In some embodiments, the engineered DNA polymerase comprises a polypeptide sequence comprising a substitution or substitution set provided in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1, wherein the substitution or substitution set is relative to the reference sequence of SEQ ID NO: 2, 178, 994, 1858, 2176, 2310, 2994, 3796, 4182, 5094, and/or 5136.

In some embodiments, the engineered DNA polymerase comprises a polypeptide sequence having at least 75%, 80%, 85%, 86%, 87%, 88%, 89%, 90%, 91%, 92%, 93%, 94%, 95%, 96%, 97%, 98%, 99% or more sequence identity to a sequence comprising residues 12 to 841 of an engineered DNA polymerase variant set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1.

In some embodiments, the engineered DNA polymerase comprises a polypeptide sequence having at least 75%, 80%, 85%, 86%, 87%, 88%, 89%, 90%, 91%, 92%, 93%, 94%, 95%, 96%, 97%, 98%, 99% or more sequence identity to a sequence of an engineered DNA polymerase variant set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1.

In some embodiments, the engineered DNA polymerase comprises a polypeptide sequence comprising residues 12 to 841 of a sequence selected from the even numbered sequences of SEQ ID NOS: 4-3258 and 3268-6646.

In some embodiments, the engineered DNA polymerase comprises a polypeptide sequence comprising residues 12 to 841 of a sequence selected from the even numbered sequences of SEQ ID NOS: 4-3258 and 3268-6646, wherein the polypeptide optionally has 1, 2, 3, 4, 5, 6, 7, 8, 9, or up to 10 substitutions in the polypeptide sequence.

In some embodiments, the engineered DNA polymerase comprises a polypeptide comprising 1, 2, 3, 4, up to 5 substitutions in the polypeptide sequence.

In some embodiments, the engineered DNA polymerase comprises substitutions that are conservative substitutions.

In some embodiments, the engineered DNA polymerase comprises a polypeptide sequence comprising a sequence comprising residues 12 to 841 of SEQ ID NO: 178, 994, 1858, 2176, 2310, 2994, 3796, 4182, 5094, and/or 5136.

In some embodiments, the engineered DNA polymerase comprises a polypeptide sequence comprising a sequence of SEQ ID NO: 178, 994, 1858, 2176, 2310, 2994, 3796, 4182, 5094, and/or 5136.

In some embodiments, the foregoing substitutions can be in addition to any of the above described embodiments.

In some embodiments, the engineered DNA polymerase comprises a fusion protein.

In some embodiments, the engineered DNA polymerase has DNA polymerase activity.

In some embodiments, the engineered DNA polymerase has at least one improved property as compared to the DNA polymerase of SEQ ID NO: 2.

In some embodiments, the engineered DNA polymerase has at least one improved property, as compared to the DNA polymerase of SEQ ID NO: 2, wherein the improved property is selected from increased activity, increased stability, increased thermostability, increased processivity, increased fidelity, increased thermotolerance, increase resistance to inhibitors, and increased product yield in a PCR reaction.

In some embodiments, the engineered DNA polymerase has at least one improved property, as compared to a wild-type DNA polymerase; the DNA polymerase comprising residues 12 to 841 of SEQ ID NO: 2; or the DNA polymerase comprising the sequence of SEQ ID NO: 2. In some embodiments, the wild-type DNA polymerase is selected from Pfu DNA polymerase from Pyrococcus furiosus, Group B DNA polymerase from Thermococcus sp. strain 2319×1, and Taq DNA polymerase from Thermus aquaticus.

In some embodiments, the engineered DNA polymerase has at least one improved property as compared to wild-type DNA polymerase; the DNA polymerase comprising residues 12 to 841 of SEQ ID NO: 2; or to the DNA polymerase comprising the sequence of SEQ ID NO: 2 (collectively referred to as “comparator reference DNA polymerase”), wherein the improved property is selected from increased product in polymerase chain reaction, increased fidelity, increased thermostability, increased tolerance to inhibitors, and/or increased processivity. In some embodiments, the engineered DNA polymerase produces a greater product yield in polymerase chain reactions than the comparator reference DNA polymerase. In some embodiments, the engineered DNA polymerase exhibits greater fidelity than the comparator DNA polymerase. In some additional embodiments, the engineered DNA polymerase exhibits greater thermostability than the comparator DNA polymerase. In some embodiments, the engineered DNA polymerase exhibits greater processivity than the comparator DNA polymerase. In some embodiments, the engineered DNA polymerase exhibits greater tolerance to inhibitors than the comparator DNA polymerase.

In some embodiments, the engineered DNA polymerase is purified. In some embodiments, the engineered DNA polymerase is provided in solution, or is immobilized on a substrate, such as surfaces of solid substrates or membranes or particles.

The present invention also provides polynucleotide sequences encoding the engineered DNA polymerases described and provided herein. In another aspect, the present invention provides polynucleotide sequences encoding the engineered DNA polymerases disclosed herein. In some embodiments, the polynucleotide sequence encodes at least one engineered DNA polymerase, or a functional fragment thereof, comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to a reference sequence comprising residues 12 to 841 of SEQ ID NO: 2, wherein the engineered DNA polymerase comprises one or more substitutions relative to the reference sequence comprising residues 12 to 841 of SEQ ID NO: 2. In some embodiments, the reference sequence is SEQ ID NO:2. In some embodiments, the one or more substitutions relative to the reference sequence comprising residues 12 to 841 of SEQ ID NO: 2 or the reference sequence of SEQ ID NO:2 are as described above and herein.

In some embodiments, the polynucleotide sequence comprises at least 75%, 80%, 85%, 86%, 87%, 88%, 89%, 90%, 91%, 92%, 93%, 94%, 95%, 96%, 97%, 98%, 99%, or more sequence identity to a reference polynucleotide sequence comprising nucleotide residues 34 to 2529 of SEQ ID NO: 1, 177, 993, 1857, 2175, 2309, 2993, 3795, 4181, 5093 and/or 5135, or to a reference sequence of SEQ ID NO: 1, 177, 993, 1857, 2175, 2309, 2993, 3795, 4181, 5093 and/or 5135, wherein the polynucleotide encodes an engineered DNA polymerase. In some embodiments, the polynucleotide comprises a polynucleotide sequence of nucleotide residues 34 to 2529 of SEQ ID NO: 1, 177, 993, 1857, 2175, 2309, 2993, 3795, 4181, 5093 and/or 5135. In some embodiments, the polynucleotide comprises a polynucleotide sequence of SEQ ID NO: 1, 177, 993, 1857, 2175, 2309, 2993, 3795, 4181, 5093 and/or 5135.

In some embodiments, the polynucleotide sequence comprises at least 75%, 80%, 85%, 86%, 87%, 88%, 89%, 90%, 91%, 92%, 93%, 94%, 95%, 96%, 97%, 98%, 99%, or more sequence identity to a reference polynucleotide sequence comprising nucleotide residues 34 to 2529 of the odd numbered polynucleotide sequences of SEQ ID NOS: 3-3257 and 3267-6645, wherein the polynucleotide encodes an engineered DNA polymerase. In some embodiments, the polynucleotide sequence comprising nucleotide residues 34 to 2529 of an odd numbered polynucleotide sequence of SEQ ID NOS: 3-3257 and 3267-6645. In some embodiments, the polynucleotide sequence comprises an odd numbered polynucleotide sequence of SEQ ID NOS: 3-3257 and 3267-6645.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 1, or to a reference polynucleotide sequence of SEQ ID NO: 1 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 2, or to a reference sequence of SEQ ID NO: 2.

In some embodiments, the polynucleotide sequence encodes an engineered DNA polymerase comprising a polypeptide sequence having at least 75%, 80%, 85%, 90%, 91%, 92%, 93%, 94%, 95%, 96%, 97%, 98%, 99%, or more sequence identity to a reference sequence comprising residues 12 to 841 of SEQ ID NO: 178, 994, 1858, 2176, 2310, 2994, 3796, 4182, 5094, and/or 5136, or to a reference sequence of SEQ ID NO: 178, 994, 1858, 2176, or 2310, 2994, 3796, 4182, 5094, and/or 5136.

In some embodiments, the polynucleotide sequence comprises nucleotide residues 34 to 2529 of SEQ ID NO: 1, 177, 993, 1857, 2175, 2309, 2993, 3795, 4181, 5093 and/or 5135 or comprises the polynucleotide sequence of SEQ ID NO: 1, 177, 993, 1857, 2175, 2309, 2993, 3795, 4181, 5093 and/or 5135.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 1, or to a reference polynucleotide sequence of SEQ ID NO: 1 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 1, or to a reference sequence of SEQ ID NO: 1.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 177, or to a reference polynucleotide sequence of SEQ ID NO: 177 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 178, or to a reference sequence of SEQ ID NO: 178.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 993, or to a reference polynucleotide sequence of SEQ ID NO: 993 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 994, or to a reference sequence of SEQ ID NO: 994.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 1857, or to a reference polynucleotide sequence of SEQ ID NO: 1857 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 1858, or to a reference sequence of SEQ ID NO: 1858.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 2175, or to a reference polynucleotide sequence of SEQ ID NO: 2175 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 2176, or to a reference sequence of SEQ ID NO: 2176.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 2309, or to a reference polynucleotide sequence of SEQ ID NO: 2309 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 2310, or to a reference sequence of SEQ ID NO: 2310.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 2993, or to a reference polynucleotide sequence of SEQ ID NO: 2993 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 2994, or to a reference sequence of SEQ ID NO: 2994.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 3795, or to a reference polynucleotide sequence of SEQ ID NO: 3795 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 3796, or to a reference sequence of SEQ ID NO: 3796.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 4181, or to a reference polynucleotide sequence of SEQ ID NO: 4181 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 4182, or to a reference sequence of SEQ ID NO: 4182.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 5093, or to a reference polynucleotide sequence of SEQ ID NO: 5093 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 5094, or to a reference sequence of SEQ ID NO: 5094.

In some embodiments, the polynucleotide comprises at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 5135, or to a reference polynucleotide sequence of SEQ ID NO: 5135 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 5136, or to a reference sequence of SEQ ID NO: 5136.

In some embodiments, the polynucleotide sequence comprises nucleotide residues 34 to 2529 of at least one of the odd-numbered sequences of SEQ ID NOS: 3-3257 and 3267-6645.

In some embodiments, the polynucleotide sequence comprises an odd-numbered sequence set forth in at least one of SEQ ID NOS: 3-3257 and 3267-6645.

In some embodiments, the polynucleotide sequence comprises nucleotide residues 34 to 2529 of SEQ ID NO: 7, 331, 461, or 605, or comprises the polynucleotide sequence of SEQ ID NO: 7, 331, 461, or 605.

In some embodiments, the polynucleotide sequence is codon-optimized. In some embodiments, the polynucleotide sequence is codon optimized for expression in an organism, for example bacteria or mammalian cells. In some embodiments, the polynucleotide sequence is operably linked to a control sequence.

The present invention also provides expression vectors comprising at least one polynucleotide sequence provided and described herein.

The present invention also provides host cells transformed with at least one polynucleotide and/or at least one expression vector.

The present invention also provides methods of producing an engineered DNA polymerase polypeptide in a host cell comprising culturing a host cell provided herein, under suitable culture conditions, such that at least one engineered DNA polymerase is produced.

In some additional embodiments, the methods further comprise recovering at least one engineered DNA polymerase from the culture and/or host cells.

In some additional embodiments, the methods further comprise a step of purifying the at least one engineered DNA polymerase.

The present invention also provides compositions comprising at least one engineered DNA polymerase described and provided herein.

In some embodiments, the composition comprises at least one buffer.

In some embodiments, the composition further comprises one or more nucleotide substrates.

In some embodiments, the composition further comprises one or more oligonucleotide primer substrates.

In some embodiments, the engineered DNA polymerase is used to detect a target DNA, the method comprising contacting a sample suspected of containing a target DNA with an engineered DNA polymerase of the present invention in presence of appropriate substrates under conditions suitable for DNA polymerase mediated production of a DNA complementary to the target DNA, whole or in part, and detecting presence of the complementary DNA. In some embodiments, the sample is a biological sample. In some embodiments, detecting the complementary DNA is by amplifying the complementary DNA, such as by polymerase chain reaction (PCR) or LAMP. In some embodiments, the engineered DNA polymerase can be used with a reverse transcriptase to detect a target RNA, such as by RT-PCR.

The present invention also provides methods of preparing a complementary DNA of a target DNA, comprising contacting a target DNA with an engineered DNA polymerase provided herein, in the presence of substrates under conditions suitable for production of a complementary DNA to all or a portion of the target DNA.

The present invention also provides methods for detecting presence of a target DNA, comprising contacting a sample suspected of containing a target DNA with an engineered DNA polymerase provided herein, in presence of substrates under conditions suitable for DNA polymerase mediated production of a DNA complementary to all or a portion of the target DNA, and detecting presence of the complementary DNA. In some embodiments, the complementary DNA is detected by amplifying the complementary DNA. In some further embodiments, the amplifying is by polymerase chain reaction (PCR) or isothermal amplification. In some additional embodiments, the isothermal amplification is by loop-mediated isothermal amplification (LAMP).

The present invention also provides methods of amplifying a target DNA, comprising contacting a target DNA with a DNA polymerase provided herein, in presence of substrates under conditions suitable for amplifying the target DNA. In some embodiments, the conditions are for a polymerase chain reaction. In some additional embodiments, the conditions are for LAMP.

The present invention also provides methods of sequencing a target DNA, comprising contacting a target DNA with a DNA polymerase provided herein, in presence of substrates appropriate for sequencing under conditions suitable for DNA polymerase mediated extension of a complementary DNA of the target DNA, and determining the sequence of the target DNA.

The present invention also provides kits comprising at least one engineered DNA polymerase provided herein. In some embodiments, the kit further comprises at least one buffer, nucleotide substrate, and/or an oligonucleotide primer substrate. In some additional embodiments, the kit further comprises a second DNA polymerase (e.g., a thermostable DNA polymerase or a reverse transcriptase). Indeed, in some embodiments, the kits comprise at least one additional enzyme, such as a reverse transcriptase.

### DESCRIPTION OF THE INVENTION

The present invention provides engineered DNA polymerase polypeptides and compositions thereof, as well as polynucleotides encoding the engineered DNA polymerase polypeptides. The disclosure also provides methods for using of the engineered DNA polymerase polypeptides and compositions thereof for diagnostic and other purposes. In some embodiments, the engineered DNA polymerase polypeptides provide enhanced polymerization activity, high replication fidelity, and/or high processivity particularly under conditions involving low concentrations of DNA input, high-throughput analysis, and/or sequencing reaction conditions. In addition, the DNA polymerases exhibit increased tolerance to inhibitors, as compared to wild-type DNA polymerases.

In some embodiments, the engineered DNA polymerases of the present invention find use in diagnostic and research applications using small amounts of DNA from samples, including cell-free DNA, circulating tumor DNA, DNA isolated from circulating tumor cells, circulating fetal DNA, DNA isolated from virally infected cells, fine-needle aspirates, or single cells isolated by FACS (fluorescence activated cell sorting), laser-capture microscopy, or microfluidic devices. However, it is not intended that the sample used with the present invention be limited to any particular sample type, as any suitable sample, including those with low DNA concentrations, finds use with the present invention.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the application as a whole. All patents, patent applications, articles and publications mentioned herein, both supra and infra, are hereby expressly incorporated herein by reference.

As used herein, the singular “a”, “an,” and “the” include the plural references, unless the context clearly indicates otherwise.

As used herein, the term “comprising” and its cognates are used in their inclusive sense (i.e., equivalent to the term “including” and its corresponding cognates).

It is to be further understood that where description of embodiments use the term “comprising” and its cognates, the embodiments can also be described using language “consisting essentially of” or “consisting of.”

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below.

Unless otherwise indicated, nucleic acids are written left to right in 5′ to 3′ orientation; amino acid sequences are written left to right in amino to carboxy orientation, respectively.

As used herein, the “EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

As used herein, “ATCC” refers to the American Type Culture Collection whose biorepository collection includes genes and strains.

As used herein, “NCBI” refers to National Center for Biological Information and the sequence databases provided therein.

As used herein, the term “DNA” refers to deoxyribonucleic acid.

As used herein, the term “RNA” refers to ribonucleic acid.

As used herein, the terms “fusion protein,” and “chimeric protein” and “chimera” refer to hybrid proteins created through the joining of two or more genes that originally encoded separate proteins. In some embodiments, fusion proteins are created by recombinant technology (e.g., molecular biology techniques known in the art).

As used herein, the term “polymerase” refers to a class of enzymes that polymerize nucleoside triphosphates. Polymerases use a template nucleic acid strand to synthesize a complementary nucleic acid strand. The template strand and synthesized nucleic acid strand can independently be either DNA or RNA. Polymerases known in the art include but are not limited to DNA polymerases (e.g., E. coli DNA poll, T. aquaticus DNA polymerase (Taq), DNA-dependent RNA polymerases, and reverse transcriptases). As used herein, the polymerase is a polypeptide or protein containing sufficient amino acids to carry out a desired enzymatic function of the polymerase. In some embodiments, the polymerase does not contain all of the amino acids found in the native enzyme, but only those which are sufficient to allow the polymerase to carry out a desired catalytic activity, including but not limited to 5′-3′ polymerization, 5′-3′ exonuclease, and 3′-5′ exonuclease activities.

As used herein, the term “DNA polymerase activity,” “synthetic activity,” and “polymerase activity” are used interchangeably herein, and refer to the ability of an enzyme to synthesize new DNA strands by the incorporation of deoxynucleoside triphosphates.

As used herein, the terms “duplex” and “ds” refer to a double-stranded nucleic acid (e.g., DNA) molecule comprised of two single-stranded polynucleotides that are complementary in their sequence (A pairs to T, C pairs to G), arranged in an antiparallel 5′ to 3′ orientation, and held together by hydrogen bonds between the nucleobases (i.e., adenine [A], guanine [G], cytosine [C], and thymine [T]).

As used here, the terms “protein,” “polypeptide,” and “peptide” are used interchangeably to denote a polymer of at least two amino acids covalently linked by an amide bond, regardless of length or post-translational modification (e.g., glycosylation or phosphorylation).

As used herein, the term “amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

As used herein, the terms “engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

As used herein, “wild-type” and “naturally-occurring” refer to the form found in nature. For example, a wild-type polypeptide or polynucleotide sequence is a sequence present in an organism that can be isolated from a source in nature and which has not been intentionally modified by human manipulation.

As used herein, “coding sequence” refers to that part of a nucleic acid (e.g., a gene) that encodes an amino acid sequence of a protein.

As used herein, the term “percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (See e.g., Altschul et al., J. Mol. Biol., 1990, 215:403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (See, Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

As used herein, “reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence based on SEQ ID NO: 2, having a valine at the residue corresponding to X200” (or “a reference sequence based on SEQ ID NO: 2, having a valine at the residue corresponding to position 712”) refers to a reference sequence in which the corresponding residue at position X200 in SEQ ID NO: 2 (e.g., an alanine), has been changed to valine.

As used herein, “comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered DNA polymerase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the sequence is tagged (e.g., with a histidine tag).

As used herein, “mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced (e.g., substitution of an aspartic acid with tryptophan). In some alternative embodiments, the mutation comprises an addition, such that an amino acid is added to the original polypeptide sequence. In some further embodiments, the mutation comprises a deletion, such that an amino acid is deleted from the original polypeptide sequence. Any number of mutations may be present in a given sequence.

As used herein, “amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X200 as compared to SEQ ID NO: 2” (or a “residue difference at position 200 as compared to SEQ ID NO: 2”) refers to a difference of the amino acid residue at the polypeptide position corresponding to position 200 of SEQ ID NO: 2. Thus, if the reference polypeptide of SEQ ID NO: 2 has an alanine at position 200, then a “residue difference at position X200 as compared to SEQ ID NO: 2” refers to an amino acid substitution of any residue other than alanine at the position of the polypeptide corresponding to position 200 of SEQ ID NO: 2. In most instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present invention can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X15A/X15G, X15A/G, or V15A/G). The present invention includes engineered polypeptide sequences comprising one or more amino acid differences that include either/or both conservative and non-conservative amino acid substitutions, as well as insertions and deletions of amino acids in the sequence.

As used herein, the terms “amino acid substitution set” and “substitution set” refers to a group of amino acid substitutions within a polypeptide sequence. In some embodiments, substitution sets comprise 1, 2, 3, 4, 5, 6, 7, 8, 9, 10, 11, 12, 13, 14, 15, or more amino acid substitutions. In some embodiments, a substitution set refers to the set of amino acid substitutions that is present in any of the variant DNA polymerase polypeptides listed in any of the Tables in the Examples. In these substitution sets, the individual substitutions are separated by a semicolon (“;”; e.g., G11D; F287L) or slash (“/”; e.g., G11D/F287L). In some embodiments, the “substitution” comprises the deletion of an amino acid.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered polymerase enzyme. Deletions can be directed to the internal portions and/or terminal portions of the polypeptide. In various embodiments, the deletion can comprise a continuous segment or can be discontinuous. Deletions are indicated by “−”, and may be present in substitution sets.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

As used herein, “functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered DNA polymerase of the present invention) and that retains substantially all of the activity of the full-length polypeptide.

As used herein, “isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant DNA polymerase polypeptides may be present within a cell, present in the cellular medium, or prepared in various forms, such as lysates or isolated preparations. As such, in some embodiments, the recombinant DNA polymerase polypeptides provided herein are isolated polypeptides.

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure DNA polymerase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant DNA polymerase polypeptides are substantially pure polypeptide compositions.

As used herein, “improved enzyme property” refers to an engineered DNA polymerase polypeptide that exhibits an improvement in any enzyme property as compared to a reference DNA polymerase polypeptide, such as a wild-type DNA polymerase polypeptide (e.g., the wild-type DNA polymerase polypeptide sequence of residues 12 to 841 of SEQ ID NO: 2) or another engineered DNA polymerase polypeptide. Improved properties include but are not limited to such properties as increased protein expression, increased thermoactivity, increased thermostability, increased stability, increased enzymatic activity, increased substrate specificity and/or affinity, increased specific activity, increased resistance to substrate and/or end-product inhibition, increased tolerance to the presence of inhibitors, increased chemical stability, improved chemoselectivity, improved solvent stability, increased tolerance to acidic pH, increased tolerance to proteolytic activity (i.e., reduced sensitivity to proteolysis), increased solubility, increased fidelity, increased processivity, and altered temperature profile.

As used herein, “increased enzymatic activity” and “enhanced catalytic activity” refer to an improved property of the engineered DNA polymerase polypeptides, which can be represented by an increase in specific activity (e.g., product produced/time/weight protein) and/or an increase in percent conversion of the substrate to the product (e.g., percent conversion of starting amount of substrate to product in a specified time period using a specified amount of DNA polymerase) as compared to the reference DNA polymerase enzyme (e.g., wild-type DNA polymerase and/or another engineered DNA polymerase). Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than the naturally occurring DNA polymerase or another engineered DNA polymerase from which the DNA polymerase polypeptides were derived.

As used herein, the terms “proteolytic activity” and “proteolysis” used interchangeably herein refer to the breakdown of proteins into smaller polypeptides or amino acids. The breakdown of proteins is generally the result of hydrolysis of the peptide bond by protease (proteinase) enzymes. Protease enzymes include but are not limited to pepsin, trypsin, chymotrypsin, elastase; carboxypeptidase A and B, and peptidases (e.g., amino peptidase, dipeptidase and enteropeptidase).

As used herein, the phrases “reducing sensitivity to proteolysis” and “reducing proteolytic sensitivity” are used interchangeably herein mean that an engineered DNA polymerase polypeptide according to the invention will have a higher enzyme activity compared to a reference DNA polymerase in a standard assay (e.g., as disclosed in the Examples) after treatment with one or more proteases.

As used herein, “conversion” refers to the enzymatic conversion (or biotransformation) of substrate(s) to the corresponding product(s). “Percent conversion” refers to the percent of the substrate that is converted to the product within a period of time under specified conditions. Thus, the “enzymatic activity” or “activity” of a DNA polymerase polypeptide can be expressed as “percent conversion” of the substrate to the product in a specific period of time.

As used herein, “hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition comprises hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

As used herein, “codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the DNA polymerase enzymes are codon optimized for optimal production from the host organism selected for expression.

As used herein, “control sequence” refers herein to include all components that are necessary or advantageous for the expression of a polynucleotide and/or polypeptide of the present invention. Each control sequence may be native or foreign to the nucleic acid sequence encoding the polypeptide. Such control sequences include, but are not limited to, leaders, polyadenylation sequences, propeptide sequences, promoter sequences, signal peptide sequences, initiation sequences, and transcription terminators. At a minimum, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

As used herein, “operably linked” refers to a configuration in which a control sequence is appropriately placed (i.e., in a functional relationship) at a position relative to a polynucleotide of interest such that the control sequence directs or regulates the expression of the polynucleotide encoding a polypeptide of interest.

As used herein, “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

As used herein, “suitable reaction conditions” or “suitable conditions” refers to those conditions in the enzymatic conversion reaction solution (e.g., ranges of enzyme loading, substrate loading, temperature, pH, buffers, co-solvents, etc.) under which a DNA polymerase polypeptide of the present invention is capable of converting a substrate to the desired product compound. Exemplary “suitable reaction conditions” are provided herein (See, the Examples).

As used herein, “loading”, such as in “compound loading” or “enzyme loading” refers to the concentration or amount of a component in a reaction mixture at the start of the reaction. “Substrate” in the context of an enzymatic conversion reaction process refers to the compound or molecule acted on by the DNA polymerase polypeptide.

As used herein, “product” in the context of an enzymatic conversion process refers to the compound or molecule resulting from the action of the DNA polymerase polypeptide on the substrate.

As used herein, “culturing” refers to the growing of a population of microbial cells under suitable conditions using any suitable medium (e.g., liquid, gel, or solid).

Recombinant polypeptides (e.g., DNA polymerase enzyme variants) can be produced using any suitable methods known the art. For example, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (regio-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased thermal stability, and/or acidic pH stability, etc.). In some embodiments, “recombinant DNA polymerase polypeptides” (also referred to herein as “engineered DNA polymerase polypeptides,” “engineered DNA polymerases,” “variant DNA polymerase enzymes,” and “DNA polymerase variants”) find use.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature.

As used herein, the terms “host cell” and “host strain” refer to suitable hosts for expression vectors comprising DNA provided herein (e.g., a polynucleotide sequences encoding at least one DNA polymerase variant). In some embodiments, the host cells are prokaryotic or eukaryotic cells that have been transformed or transfected with vectors constructed using recombinant DNA techniques as known in the art.

As used herein, the term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide.

In some embodiments, analogues include non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

As used herein, the term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

As used herein, “cell-free DNA” refers to DNA circulating freely in the bloodstream and is not contained by or associated with cells. In some embodiments, cell-free DNA comprises DNA originally derived and released from normal somatic or germ line cells, cancer cells, fetal cells, microbial cells, or viruses.

As used herein, “amplification” refers to nucleic acid replication. In some embodiments, the term refers to replication of specific template nucleic acid.

As used herein, “polymerase chain reaction” and “PCR” refer to the methods described in U.S. Pat. Nos. 4,683,195 and 4,6884,202, hereby incorporated by reference. These methods find use in increasing the concentration of a segment of a target sequence or an entire target sequence in a mixture or purified DNA, without cloning or purification being required. The sequence of denaturation, annealing and extension constitute a “cycle.” The steps of denaturing, primer annealing, and polymerase extension can be repeated many times (i.e., multiple cycles are used), to obtain a high concentration of amplified DNA. The process is well-known in the art and numerous variations have been developed over the years since the method was first described. With PCR, it is possible to amplify a single copy of a specific target sequence to a level that is detectable by several different methodologies, including but not limited to hybridization with a labeled probe, incorporation of biotinylated primers followed by avidin-enzyme conjugate detection, incorporation of ³²P-labeled deoxyribonucleotide triphosphates (e.g., dCTP or dATP) into the amplified segment, etc. In addition to genomic DNA, any oligonucleotide sequence amenable to amplification can be copies using PCR with an appropriate set of primers. PCR products can also serve as templates for amplification.

As used herein, “target” when used in reference to a method employing a DNA polymerase, refers to the region of nucleic acid for preparation of a complementary DNA. The “target” is sorted out from other nucleic acids present in the methods using a DNA polymerase. In some embodiments, a “segment” is a region of nucleic acid within the target sequence.

As used herein, “target DNA” when used in context of a DNA polymerase refers to the DNA, all or a portion thereof, that is the object for preparation of a complementary DNA copy. The target DNA can be the whole of the DNA sequence or a portion thereof, such as a segment of the DNA sequence.

As used herein, “target RNA” refers to the RNA, all or a portion thereof, that is the object for preparation of a complementary DNA copy. The target RNA can be the whole of the RNA sequence or a portion thereof, such as a segment of the RNA sequence.

As used herein, “sample template” refers to nucleic acid originating from a sample which is analyzed for the presence of target nucleic acid. In contrast, “background template” refers to nucleic acid other than sample template that may or may not be present within a sample. Background template may be inadvertently included in the sample, it may result from carryover, or may be due to the presence of nucleic acid contaminants from which the target nucleic acid is purified. For example, in some embodiments, nucleic acids from organisms other than those to be detected may be present as background in a test sample. However, it is not intended that the present invention be limited to any specific nucleic acid samples or templates.

As used herein, “amplifiable nucleic acid” is used in reference to nucleic acids which may be amplified by any amplification method, including but not limited to PCR. In most embodiments, amplifiable nucleic acids comprise sample templates.

As used herein, “PCR product”, “PCR fragment,” and “amplification product” refer to the resultant compounds obtained after two or more cycles of PCR amplification (or other amplification method, as indicated by the context), typically comprising the steps of denaturation, annealing, and extension. The terms encompass the situation wherein there has been amplification of one or more segments of one or more target sequences.

As used herein, “amplification reagents” and “PCR reagents” refer to those reagents (e.g., deoxyribonucleotide triphosphates, buffer, etc.), needed for amplification except for the primers, nucleic acid template, and the amplification enzyme. Typically, amplification reagents, along with other reaction components are placed and contained in a reaction vessel (e.g., test tube, microwell, etc.). It is not intended that the present invention be limited to any specific amplification reagents, as any suitable reagents find use in the present invention.

As used herein, “primer” refers to an oligonucleotide (i.e., a sequence of nucleotides), whether occurring naturally or produced synthetically, recombinantly, or by amplification, which is capable of acting as a point of initiation of nucleic acid synthesis, when placed under conditions in which synthesis of a primer extension product that is complementary to a nucleic acid strand is induced (i.e., in the presence of nucleotides and an inducing agent such as DNA polymerase, and at a suitable temperature and pH). In most embodiments, primers a single-stranded, but in some embodiments, they are double-stranded. In some embodiments, the primers are of sufficient length to prime the synthesis of extension products in the presence of DNA polymerase. The exact primer length depends upon many factors, as known to those skilled in the art.

As used herein, “probe” refers to an oligonucleotide (i.e., a sequence of nucleotides), whether occurring naturally or produced synthetically, recombinantly, or by amplification, which is capable of hybridizing to another oligonucleotide of interest. Probes find use in the detection, identification, and/or isolation of particular gene sequences of interest. In some embodiments, probes are labeled with a “reporter molecule” (also referred to as a “label”) that aids in the detection of the probe in a suitable detection system (e.g., fluorescent, radioactive, luminescent, enzymatic, and other systems). It is not intended that the present invention be limited to any particular detection system or label. Primers, deoxyribonucleotides, and deoxyribonucleosides may contain labels. Indeed, it is not intended that the labeled composition of the present invention be limited to any particular component. Illustrative labels include, but are not limited to 32P, 35S, and fluorescent molecules (e.g., fluorescent dyes, including but not limited to green fluorescent protein).

As used herein, “fidelity,” when used in reference to a polymerase is intended to refer to the accuracy of template-directed incorporation of complementary bases in a synthesized DNA strand relative to the template strand. Typically, fidelity is measured based on the frequency of incorporation of incorrect bases in the newly synthesized nucleic acid strand. The incorporation of incorrect bases can result in point mutations, insertions, or deletions. Fidelity can be calculated according to any method known in the art (See e.g., Tindall and Kunkel, Biochem., 1988, 27:6008-6013; and Barnes, Gene, 1992, 112:29-35). A polymerase or polymerase variant can exhibit either high fidelity or low fidelity. As used herein, “high fidelity” refers to polymerases with a frequency of accurate base incorporation that exceeds a predetermined value. As used herein, the term “low fidelity” refers to polymerases with a frequency of accurate base incorporation that is lower than a predetermined value. In some embodiments, the predetermined value is a desired frequency of accurate base incorporation or the fidelity of a known polymerase (i.e., a reference polymerase).

As used herein, “altered fidelity” refers to the fidelity of a polymerase variant that differs from the fidelity of the parent polymerase from which the polymerase variant was derived. In some embodiments, the altered fidelity is higher than the fidelity of the parent polymerase, while in some other embodiments, the altered fidelity is lower than the fidelity of the parent polymerase. Altered fidelity can be determined by assaying the parent and variant polymerases and comparing their activities using any suitable assay known in the art.

As used herein, the term “processivity” refers to the ability of a nucleic acid modifying enzyme, such as a DNA polymerase, to remain bound to the template or substrate and perform multiple modification reactions. Processivity is generally measured by the number of catalytic events that take place per binding event.

As used herein, “altered processivity” refers to the processivity of polymerase, or variants thereof that differ from the processivity of the parent polymerase from which the variant was derived. In some embodiments, the altered processivity is higher than the processivity of the parent enzyme, while in some other embodiments, the altered processivity is lower than the processivity of the parent enzyme. Altered processivity can be determined by assaying the parent and variant enzymes and comparing their activities using any suitable assay known in the art.

The term “subject” encompasses mammals such as humans, non-human primates, livestock, companion animals, and laboratory animals (e.g., rodents and lagamorphs). It is intended that the term encompass females as well as males.

As used herein, the term “patient” means any subject that is being assessed for, treated for, or is experiencing disease.

As used herein, the term “sample” refers to a material or substance for reaction with a DNA polymerase, for example, such as for detecting presence of a target nucleic acid or preparing a DNA copy of a target nucleic acid for sequencing or generation of cDNA libraries. In some embodiments, the sample is a “biological sample,” which refers to sample of biological tissue or fluid. Such samples are typically from humans, but include tissues isolated from non-human primates, or rodents, e.g., mice, and rats, and includes sections of tissues such as biopsy and autopsy samples, frozen sections taken for histological purposes, blood, plasma, serum, sputum, stool, tears, mucus, hair, skin, etc. A “biological sample” also refers to a cell or population of cells or a quantity of tissue or fluid from organisms. In some embodiments, the biological sample has been removed from an animal, but the term “biological sample” can also refer to cells or tissue analyzed in vivo, i.e., without removal from the animal. Typically, a “biological sample” will contain cells from the animal or of organisms, but the term can also refer to noncellular biological material, such as noncellular fractions of blood, saliva, or urine. Numerous types of biological samples can be used with the enzymes, compositions, and method in the present invention, including, but not limited to, a tissue biopsy, a blood sample, a buccal scrape, a saliva sample, or a nipple discharge. As used herein, a “tissue biopsy” refers to an amount of tissue removed from an animal, preferably a human, for diagnostic analysis. In a patient with cancer, tissue may be removed from a tumor, allowing the analysis of cells within the tumor.

“Tissue biopsy” can refer to any type of biopsy, such as needle biopsy, fine needle biopsy, surgical biopsy, etc.

### Engineered DNA Polymerase Polypeptides

In some embodiments herein, when a particular DNA polymerase variant (i.e., an engineered DNA polymerase polypeptide) is referred to by reference to modification of particular amino acid residues in the sequence of a wild-type DNA polymerase or reference DNA polymerase polypeptide, it is to be understood that variants of another DNA polymerase modified in the equivalent position(s) (as determined from the optional amino acid sequence alignment between the respective amino acid sequences) are encompassed herein. For example, for a substitution at specified amino acid position(s) numbered in reference to SEQ ID: 2, an equivalent amino acid position(s) can be readily ascertained for another reference sequence, such as a reference sequence comprising residues 12 to 841 of SEQ ID NO: 2, 178, 994, 1858, 2176, 2310, 2994, 3796, 4182, 5094, and/or 5136, or such as a references sequence of SEQ ID NO: 2, 178, 994, 1858, 2176, 2310, 2994, 3796, 4182, 5094, and/or 5136.

In some embodiments, the engineered DNA polymerase polypeptide variants of the present invention are useful in performing polymerase reactions, including preparing a complementary DNA of a target DNA target/template. In some embodiments, the engineered DNA polymerase variants of the present invention find use in the efficient creation of complementary DNA from DNA templates, whole or in part, such as in sequencing (e.g., NGS sequencing), amplification (e.g., PCR), and diagnostic methods, such as for detecting a target nucleic acid. These engineered DNA polymerase variants can be used in solution, as well as in immobilized embodiments. In some embodiments, the engineered DNA polymerase can be prepared and used as non-fusion polypeptides or as fusion polypeptides.

In some embodiments, an engineered DNA polymerase or a functional fragment thereof of the present invention comprises a polypeptide comprising at least 70%, at least 75%, or at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a reference sequence comprising residues 12 to 841 of SEQ ID NO: 2, wherein the engineered DNA polymerase comprises one or more substitutions relative to the reference sequence comprising residues 12 to 841 of SEQ ID NO:2. In some embodiments, an engineered DNA polymerase or a functional fragment thereof of the present invention comprises a polypeptide comprising at least about 70%, at least about 75%, or at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a reference sequence comprising residues 12 to 841 of SEQ ID NO: 2, wherein the engineered DNA polymerase comprises one or more substitutions relative to the reference sequence comprising residues 12 to 841 of SEQ ID NO:2.

In some embodiments, an engineered DNA polymerase or a functional fragment thereof of the present invention comprises a polypeptide comprising at least 70%, at least 75%, or at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a reference sequence of SEQ ID NO: 2, wherein the recombinant DNA polymerase comprises one or more substitutions relative to the reference sequence of SEQ ID NO:2.

In some embodiments, the engineered DNA polymerase or a functional fragment thereof, comprises a polypeptide sequence having at least 80% or more sequence identity to reference sequence comprising residues 12 to 841 of SEQ ID NO: 2, wherein the polypeptide sequence comprises one or more substitutions relative to the reference sequence comprising residues 12 to 841 of SEQ ID NO: 2.

In some embodiments, the engineered DNA polymerase or a functional fragment thereof comprises a polypeptide sequence having at least 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2, wherein the polypeptide sequence comprises one or more substitutions relative to the reference sequence comprising residues 12 to 841 of SEQ ID NO:2.

In some embodiments, the reference sequence comprises the sequence of SEQ ID NO: 2. In some embodiments, the engineered DNA polymerase or a functional fragment thereof, comprises a polypeptide sequence having at least 80% or more sequence identity to a reference sequence of SEQ ID NO: 2, wherein the polypeptide sequence comprises one or more substitutions relative to SEQ ID NO: 2.

In some embodiments, the engineered DNA polymerase comprises a polypeptide sequence having at least 85%, 86%, 87%, 88%, 89%, 90%, 91%, 92%, 93%, 94%, 95%, 96%, 97%, 98%, 99%, or more sequence identity to the reference sequence of SEQ ID NO: 2, wherein the polypeptide sequence comprises one or more substitutions relative to the reference sequence of SEQ ID NO: 2. In some embodiments, the engineered DNA polymerase comprises a polypeptide sequence having at least about 85%, about 86%, about 87%, about 88%, about 89%, about 90%, about 91%, about 92%, about 93%, about 94%, about 95%, about 96%, about 97%, about 98%, about 99%, or more sequence identity to the reference sequence of SEQ ID NO: 2, wherein the polypeptide sequence comprises one or more substitutions relative to the reference sequence of SEQ ID NO: 2.

In some embodiments, the engineered DNA polymerase comprises at least one substitutions or substitution sets selected from 11/287, 15/633, 18/145/255, 20/602, 29, 50/255/557/685, 110, 113/170/194/235/249/319/645/659/660/674/699, 130, 145, 145/255/287/391/472/476/557, 145/255/287/391/476/513, 145/255/287/391/557/685/743/758, 145/255/391/398/472/476/513/557/602/660, 145/255/391/398/472/557/685, 145/255/391/398/476/557/758, 145/255/391/398/513/658/685/743, 145/255/391/472/476/685/743, 145/255/391/472/557/685, 145/255/391/476/557/602/660/743, 145/255/391/476/557/658/660/743, 145/255/391/476/758, 145/255/391/513/557/743/758, 145/255/391/557/758, 145/255/398/472/476/513/557/658/660, 145/255/472/476/557/685/743/758, 145/255/472/513/557/602/660/743, 145/255/513, 145/255/513/602, 145/255/557, 145/255/557/658/660/685, 145/255/743/758, 145/267/391/398/513/557/658/685/743, 145/287/391/472/557/602/658/660/685, 145/287/391/472/602/658, 145/287/391/476/513/557/602, 145/287/391/476/658/660/685, 145/287/658/758, 145/391, 145/391/398/476/758, 145/391/472/476/513/557/658/685/758, 145/391/472/476/557/602/660/685/743, 145/391/472/476/557/743/758, 145/391/472/476/658/660/758, 145/391/472/476/658/685/758, 145/391/472/476/758, 145/391/513/557/602, 145/391/513/557/658/685, 145/391/513/658/743/758, 145/391/513/758, 145/391/557, 145/391/557/658/758, 145/391/557/660/685, 145/391/557/743, 145/398/472/557/660, 145/476/557/660/685/743/758, 145/513/685, 145/557/658, 145/557/685/758, 145/685, 161/162, 161/162/170/194/235/423/699, 161/162/170/235/249/252, 161/162/194/269/605/660, 161/162/269/476/660, 161/162/352/434/605, 161/162/659/660/674, 161/170/235/249/252/434, 161/170/235/269/319, 161/170/235/319/412, 161/170/252/352/412/434/699, 161/170/476, 161/194/198/235/252/423/434/605/645/699, 161/194/235/249/352/412/434/674/699, 161/194/252, 161/194/252/323/352/412/605/659/660/674/699, 161/194/252/412/605, 161/194/434, 161/235/249/319/412/605/659, 161/235/252/319/352/645/674/699, 161/235/352/423/645/674, 161/235/645/699, 161/249/412/476/605/645/659/660/674, 161/252/319/352/412/423/476/605/645, 161/252/319/674, 161/269/434/699, 161/319/660, 161/352, 161/423/645, 161/605, 162, 162/170/194/235/249/269/674/699, 162/170/412/434/476/605, 162/194/249/645/659, 162/235/269/423/476/605/645/659, 162/252/352/412/434/645/660/699, 162/269/352/412/476/605, 162/352, 162/423/645, 162/605/645, 164, 168, 170, 170/194/198/235/674/699, 170/194/235/252/269/323/412/423/476/605/645/660/674, 170/194/235/319/412/476/699, 170/194/269/319/412/423/476/605/645, 170/194/319/352/476/699, 170/235/249/352/645, 170/235/269/352, 170/235/269/434/674, 170/235/319/352/412/434/476/645/660/674, 170/235/352/674/699, 170/235/352/699, 170/235/412, 170/235/645, 170/235/660, 170/249/252/269/352/412/423/434/476/659, 170/249/252/269/423/659, 170/249/412, 170/252/352/423/674/699, 170/269/352/423/476/605/659/660/699, 170/269/476/645, 170/412/423/476/605/645/660/674, 170/476/674, 175, 177, 194, 194/198/235/252/323/412/605/645, 194/198/252/319/423/645/674, 194/235/252, 194/235/252/319/423, 194/235/605, 194/235/645, 194/235/699, 194/249/252/645/699, 194/249/412/476/605/645/699, 194/252/269/319/434/605/645/659/674/699, 194/252/319/412/605, 194/269/319/412/605/674/699, 194/319/352/605/660, 194/323/699, 194/352/476/659, 194/352/605/645/699, 194/434, 194/645/659, 197, 198/674/699, 202/268, 217, 227, 235, 235/249/252/323/352/476, 235/249/252/412/605/645/699, 235/249/252/423/699, 235/249/252/434/605/645/699, 235/252, 235/252/269/352/434/605/674/699, 235/252/269/352/476/645/674, 235/252/269/423/605/645/660/674, 235/269/323/412/434/659/674/699, 235/269/674/699, 235/319/605/645/674, 235/352, 235/352/412/659/660/674/699, 235/352/605, 235/412/645, 235/476/674, 235/605/645, 249/252/319/352/645, 249/252/319/605/660, 249/252/412/423/645/674/699, 249/412/476/605, 252/352/645/660, 252/645, 253, 255/260/398/557, 255/287, 255/287/391/472/557/658/660, 255/287/391/472/658/660/685, 255/287/391/476/557, 255/287/391/658, 255/391, 255/391/398/476/557, 255/391/398/476/602/658/660/685, 255/391/398/513/557/658/743/758, 255/391/472/513/758, 255/391/472/557/658/660/685/743/758, 255/391/472/557/658/660/758, 255/391/472/658/660/758, 255/391/472/685, 255/391/476/513/557/602/685/758, 255/391/476/557/602/660/758, 255/391/476/557/658, 255/391/476/758, 255/391/513/602/658/660, 255/391/557, 255/391/602, 255/391/685/758, 255/391/743, 255/398/472/476/557/658, 255/398/476, 255/398/476/557/658/660/685, 255/398/513/758, 255/472/758, 255/513/557, 255/513/557/685/758, 255/557/658, 255/602, 255/660, 267, 268, 269/352, 269/352/412, 269/605, 270, 271/472, 279, 281, 287/391/472, 287/391/472/476/557/602/658/660/685/743, 287/391/476/513, 287/391/513/557/660/685/743/758, 287/391/557, 287/391/557/602/658/743, 287/391/557/685, 287/391/602, 287/685, 297, 319, 319/352/412/434/699, 319/434/476, 322, 329, 331, 339, 347, 350, 352, 352/412/605/645/699, 352/605/645/699, 352/605/659, 352/645, 354, 356, 358, 360, 361, 365, 372, 377, 379, 391, 391/398/476/685, 391/398/513/602/658/660/758, 391/451/472/513/557, 391/472/476/557/602, 391/472/513/557/602/658, 391/472/513/602/658, 391/476/513/743, 391/476/557, 391/513/658/660, 391/557, 391/557/758, 391/602, 391/602/758, 391/658, 391/660/743/758, 391/685, 396, 398, 398/472, 398/557/602/658, 412, 412/645, 412/674, 423, 423/476/605, 423/476/645/674, 423/645, 423/699, 425, 426, 435, 440, 455, 456, 467, 472, 472/476/513/743, 472/476/557/658/758, 472/513/557/658/743/758, 472/513/557/660/685, 472/513/658/660/743, 472/557/660/685/743/758, 472/758, 476, 476/557/758, 476/685, 476/758, 478/557, 479, 485, 506, 508, 513, 513/557/743, 513/557/758, 532, 533, 540, 557, 557/658/660, 557/658/685, 557/743, 564, 602, 602/685, 602/703, 605/699, 635, 643, 645, 645/659, 645/674/699, 658, 658/660, 658/685/758, 659, 660, 674, 704, 713, 718, 758, and 810, or combinations thereof, wherein the amino acid positions are relative to the reference sequence of SEQ ID NO: 2.

In some embodiments, the engineered DNA polymerase comprises one or more substitutions or substitution sets are selected from 11D/287L, 15S/633N, 18H/145H/255V, 20C/602H, 29N, 50H/255V/557R/685A, 110M, 113F/170L/194V/235E/249R/319V/645K/659P/660P/674V/699V, 130M, 145H, 145H/255V/287L/391S/472A/476L/557R, 145H/255V/287L/391S/476L/513T, 145H/255V/287L/391S/557R/685A/743T/758W, 145H/255V/391S/398R/472A/476L/513T/557R/602H/660S, 145H/255V/391S/398R/472A/557R/685A, 145H/255V/391S/398R/476L/557R/758W, 145H/255V/391S/398R/513T/658V/685A/743T, 145H/255V/391S/472A/476L/685A/743T, 145H/255V/391S/472A/557R/685A, 145H/255V/391S/476L/557R/602H/660S/743T, 145H/255V/391S/476L/557R/658V/660S/743T, 145H/255V/391S/476L/758W, 145H/255V/391S/513T/557R/743T/758W, 145H/255V/391S/557R/758W, 145H/255V/398R/472A/476L/513T/557R/658V/660S, 145H/255V/472A/476L/557R/685A/743T/758W, 145H/255V/472A/513T/557R/602H/660S/743T, 145H/255V/513T, 145H/255V/513T/602H, 145H/255V/557R, 145H/255V/557R/658V/660S/685A, 145H/255V/743T/758W, 145H/267S/391S/398R/513T/557R/658V/685A/743T, 145H/287L/391S/472A/557R/602H/658V/660S/685A, 145H/287L/391S/472A/602H/658V, 145H/287L/391S/476L/513T/557R/602H, 145H/287L/391S/476L/658V/660S/685A, 145H/287L/658V/758W, 145H/391S, 145H/391S/398R/476L/758W, 145H/391S/472A/476L/513T/557R/658V/685A/758W, 145H/391S/472A/476L/557R/602H/660S/685A/743T, 145H/391S/472A/476L/557R/743T/758W, 145H/391S/472A/476L/658V/660S/758W, 145H/391S/472A/476L/658V/685A/758W, 145H/391S/472A/476L/758W, 145H/391S/513T/557R/602H, 145H/391S/513T/557R/658V/685A, 145H/391S/513T/658V/743T/758W, 145H/391S/513T/758W, 145H/391S/557R, 145H/391S/557R/658V/758W, 145H/391S/557R/660S/685A, 145H/391S/557R/743T, 145H/398R/472A/557R/660S, 145H/476L/557R/660S/685A/743T/758W, 145H/513T/685A, 145H/557R/658V, 145H/557R/685A/758W, 145H/685A, 161V/162A, 161V/162A/170L/194V/235E/423Q/699V, 161V/162A/170L/235E/249R/252L, 161V/162A/194V/269R/605R/660P, 161V/162A/269R/476I/660P, 161V/162A/352K/434K/605R, 161V/162A/659P/660P/674V, 161V/170L/235E/249R/252L/434K, 161V/170L/235E/269R/319V, 161V/170L/235E/319V/412A, 161V/170L/252L/352K/412A/434K/699V, 161V/170L/476I, 161V/194V/198G/235E/252L/423Q/434K/605R/645K/699V, 161V/194V/235E/249R/352K/412A/434K/674V/699V, 161V/194V/252L, 161V/194V/252L/323S/352K/412A/605R/659P/660P/674V/699V, 161V/194V/252L/412A/605R, 161V/194V/434K, 161V/235E/249R/319V/412A/605R/659P, 161V/235E/252L/319V/352K/645K/674V/699V, 161V/235E/352K/423Q/645K/674V, 161V/235E/645K/699V, 161V/249R/412A/476I/605R/645K/659P/660P/674V, 161V/252L/319V/352K/412A/423Q/476I/605R/645K, 161V/252L/319V/674V, 161V/269R/434K/699V, 161V/319V/660P, 161V/352K, 161V/423Q/645K, 161V/605R, 162A, 162A/170L/194V/235E/249R/269R/674V/699V, 162A/170L/412A/434K/476I/605R, 162A/194V/249R/645K/659P, 162A/235E/269R/423Q/476I/605R/645K/659P, 162A/252L/352K/412A/434K/645K/660P/699V, 162A/269R/352K/412A/476I/605R, 162A/352K, 162A/423Q/645K, 162A/605R/645K, 162L, 164C, 168V, 170L, 170L/194V/198G/235E/674V/699V, 170L/194V/235E/252L/269R/323S/412A/423Q/476I/605R/645K/660P/674V, 170L/194V/235E/319V/412A/476I/699V, 170L/194V/269R/319V/412A/423Q/476I/605R/645K, 170L/194V/319V/352K/476I/699V, 170L/235E/249R/352K/645K, 170L/235E/269R/352K, 170L/235E/269R/434K/674V, 170L/235E/319V/352K/412A/434K/476I/645K/660P/674V, 170L/235E/352K/674V/699V, 170L/235E/352K/699V, 170L/235E/412A, 170L/235E/645K, 170L/235E/660P, 170L/249R/252L/269R/352K/412A/423Q/434K/476I/659P, 170L/249R/252L/269R/423Q/659P, 170L/249R/412A, 170L/252L/352K/423Q/674V/699V, 170L/269R/352K/423Q/476I/605R/659P/660P/699V, 170L/269R/476I/645K, 170L/412A/423Q/476I/605R/645K/660P/674V, 170L/476I/674V, 175A, 177F, 194V, 194V/198G/235E/252L/323S/412A/605R/645K, 194V/198G/252L/319V/423Q/645K/674V, 194V/235E/252L, 194V/235E/252L/319V/423Q, 194V/235E/605R, 194V/235E/645K, 194V/235E/699V, 194V/249R/252L/645K/699V, 194V/249R/412A/476I/605R/645K/699V, 194V/252L/269R/319V/434K/605R/645K/659P/674V/699V, 194V/252L/319V/412A/605R, 194V/269R/319V/412A/605R/674V/699V, 194V/319V/352K/605R/660P, 194V/323S/699V, 194V/352K/476I/659P, 194V/352K/605R/645K/699V, 194V/434K, 194V/645K/659P, 197Q, 198G/674V/699V, 202S/268K, 217T, 227E, 235E, 235E/249R/252L/323S/352K/476I, 235E/249R/252L/412A/605R/645K/699V, 235E/249R/252L/423Q/699V, 235E/249R/252L/434K/605R/645K/699V, 235E/252L, 235E/252L/269R/352K/434K/605R/674V/699V, 235E/252L/269R/352K/4761/645K/674V, 235E/252L/269R/423Q/605R/645K/660P/674V, 235E/269R/323S/412A/434K/659P/674V/699V, 235E/269R/674V/699V, 235E/319V/605R/645K/674V, 235E/352K, 235E/352K/412A/659P/660P/674V/699V, 235E/352K/605R, 235E/412A/645K, 235E/476I/674V, 235E/605R/645K, 249R/252L/319V/352K/645K, 249R/252L/319V/605R/660P, 249R/252L/412A/423Q/645K/674V/699V, 249R/412A/476I/605R, 252L/352K/645K/660P, 252L/645K, 253D, 255V/260A/398R/557R, 255V/287L, 255V/287L/391S/472A/557R/658V/660S, 255V/287L/391S/472A/658V/660S/685A, 255V/287L/391S/476L/557R, 255V/287L/391S/658V, 255V/391S, 255V/391S/398R/476L/557R, 255V/391S/398R/476L/602H/658V/660S/685A, 255V/391S/398R/513T/557R/658V/743T/758W, 255V/391S/472A/513T/758W, 255V/391S/472A/557R/658V/660S/685A/743T/758W, 255V/391S/472A/557R/658V/660S/758W, 255V/391S/472A/658V/660S/758W, 255V/391S/472A/685A, 255V/391S/476L/513T/557R/602H/685A/758W, 255V/391S/476L/557R/602H/660S/758W, 255V/391S/476L/557R/658V, 255V/391S/476L/758W, 255V/391S/513T/602H/658V/660S, 255V/391S/557R, 255V/391S/602H, 255V/391S/685A/758W, 255V/391S/743T, 255V/398R/472A/476L/557R/658V, 255V/398R/476L, 255V/398R/476L/557R/658V/660S/685A, 255V/398R/513T/758W, 255V/472A/758W, 255V/513T/557R, 255V/513T/557R/685A/758W, 255V/557R/658V, 255V/602H, 255V/660S, 267Y, 268K, 269R/352K, 269R/352K/412A, 269R/605R, 270Y, 271H/472A, 2791, 281M, 287L/391S/472A, 287L/391S/472A/476L/557R/602H/658V/660S/685A/743T, 287L/391S/476L/513T, 287L/391S/513T/557R/660S/685A/743T/758W, 287L/391S/557R, 287L/391S/557R/602H/658V/743T, 287L/391S/557R/685A, 287L/391S/602H, 287L/685A, 297T, 319V, 319V/352K/412A/434K/699V, 319V/434K/4761, 322V, 329Q, 331S, 339E, 3471, 350F, 352G, 352K, 352K/412A/605R/645K/699V, 352K/605R/645K/699V, 352K/605R/659P, 352K/645K, 354I, 356N, 358N, 360F, 361I, 3651, 372N, 377Q, 3791, 391S, 391S/398R/476L/685A, 391S/398R/513T/602H/658V/660S/758W, 391S/451V/472A/513T/557R, 391S/472A/476L/557R/602H, 391S/472A/513T/557R/602H/658V, 391S/472A/513T/602H/658V, 391S/476L/513T/743T, 391S/476L/557R, 391S/513T/658V/660S, 391S/557R, 391S/557R/758W, 391S/602H, 391S/602H/758W, 391S/658V, 391S/660S/743T/758W, 391S/685A, 396K, 398R, 398R/472A, 398R/557R/602H/658V, 398T, 412A, 412A/645K, 412A/674V, 423Q, 423Q/4761/605R, 423Q/4761/645K/674V, 423Q/645K, 423Q/699V, 423V, 425F, 426V, 435Y, 440T, 455V, 456K, 467N, 472A, 472A/476L/513T/743T, 472A/476L/557R/658V/758W, 472A/513T/557R/658V/743T/758W, 472A/513T/557R/660S/685A, 472A/513T/658V/660S/743T, 472A/557R/660S/685A/743T/758W, 472A/758W, 476I, 476L, 476L/557R/758W, 476L/685A, 476L/758W, 478H/557R, 479M, 485K, 506N, 508N, 513T, 513T/557R/743T, 513T/557R/758W, 532M, 533D, 540N, 557R, 557R/658V/660S, 557R/658V/685A, 557R/743T, 564E, 602H, 602H/685A, 602H/703R, 605R/699V, 635K, 643H, 645K, 645K/659P, 645K/674V/699V, 658V, 658V/660S, 658V/685A/758W, 659E, 660S, 674V, 704K, 713Q, 718D, 758W, and 810K, or combinations thereof, wherein the amino acid positions are relative to SEQ ID NO: 2.

In some embodiments, the engineered DNA polymerase comprises one or more substitutions or substitution sets are selected from G11D/F287L, L15S/G633N, P18H/Q145H/S255V, G20C/K602H, H29N, Q50H/S255V/P557R/H685A, L110M, L113F/R170L/A194V/A235E/K249R/R319V/R645K/S659P/A660P/1674V/A699V, L130M, Q145H, Q145H/S255V/F287L/P391S/V472A/M476L/P557R, Q145H/S255V/F287L/P391S/M476L/G513T, Q145H/S255V/F287L/P391S/P557R/H685A/N743T/F758W, Q145H/S255V/P391S/G398R/V472A/M476L/G513T/P557R/K602H/A660S, Q145H/S255V/P391S/G398R/V472A/P557R/H685A, Q145H/S255V/P391S/G398R/M476L/P557R/F758W, Q145H/S255V/P391S/G398R/G513T/L658V/H685A/N743T, Q145H/S255V/P391S/V472A/M476L/H685A/N743T, Q145H/S255V/P391S/V472A/P557R/H685A, Q145H/S255V/P391S/M476L/P557R/K602H/A660S/N743T, Q145H/S255V/P391S/M476L/P557R/L658V/A660S/N743T, Q145H/S255V/P391S/M476L/F758W, Q145H/S255V/P391S/G513T/P557R/N743T/F758W, Q145H/S255V/P391S/P557R/F758W, Q145H/S255V/G398R/V472A/M476L/G513T/P557R/L658V/A660S, Q145H/S255V/V472A/M476L/P557R/H685A/N743T/F758W, Q145H/S255V/V472A/G513T/P557R/K602H/A660S/N743T, Q145H/S255V/G513T, Q145H/S255V/G513T/K602H, Q145H/S255V/P557R, Q145H/S255V/P557R/L658V/A660S/H685A, Q145H/S255V/N743T/F758W, Q145H/F267S/P391S/G398R/G513T/P557R/L658V/H685A/N743T, Q145H/F287L/P391S/V472A/P557R/K602H/L658V/A660S/H685A, Q145H/F287L/P391S/V472A/K602H/L658V, Q145H/F287L/P391S/M476L/G513T/P557R/K602H, Q145H/F287L/P391S/M476L/L658V/A660S/H685A, Q145H/F287L/L658V/F758W, Q145H/P391S, Q145H/P391S/G398R/M476L/F758W, Q145H/P391S/V472A/M476L/G513T/P557R/L658V/H685A/F758W, Q145H/P391S/V472A/M476L/P557R/K602H/A660S/H685A/N743T, Q145H/P391S/V472A/M476L/P557R/N743T/F758W, Q145H/P391S/V472A/M476L/L658V/A660S/F758W, Q145H/P391S/V472A/M476L/L658V/H685A/F758W, Q145H/P391S/V472A/M476L/F758W, Q145H/P391S/G513T/P557R/K602H, Q145H/P391S/G513T/P557R/L658V/H685A, Q145H/P391S/G513T/L658V/N743T/F758W, Q145H/P391S/G513T/F758W, Q145H/P391S/P557R, Q145H/P391S/P557R/L658V/F758W, Q145H/P391S/P557R/A660S/H685A, Q145H/P391S/P557R/N743T, Q145H/G398R/V472A/P557R/A660S, Q145H/M476L/P557R/A660S/H685A/N743T/F758W, Q145H/G513T/H685A, Q145H/P557R/L658V, Q145H/P557R/H685A/F758W, Q145H/H685A, I161V/S162A, I161V/S162A/R170L/A194V/A235E/A423Q/A699V, I161V/S162A/R170L/A235E/K249R/R252L, I161V/S162A/A194V/G269R/K605R/A660P, I161V/S162A/G269R/M476I/A660P, I161V/S162A/R352K/R434K/K605R, I161V/S162A/S659P/A660P/1674V, I161V/R170L/A235E/K249R/R252L/R434K, I161V/R170L/A235E/G269R/R319V, I161V/R170L/A235E/R319V/G412A, I161V/R170L/R252L/R352K/G412A/R434K/A699V, I161V/R170L/M476I, I161V/A194V/S198G/A235E/R252L/A423Q/R434K/K605R/R645K/A699V, I161V/A194V/A235E/K249R/R352K/G412A/R434K/1674V/A699V, I161V/A194V/R252L, I161V/A194V/R252L/P323S/R352K/G412A/K605R/S659P/A660P/1674V/A699V, I161V/A194V/R252L/G412A/K605R, I161V/A194V/R434K, I161V/A235E/K249R/R319V/G412A/K605R/S659P, I161V/A235E/R252L/R319V/R352K/R645K/1674V/A699V, I161V/A235E/R352K/A423Q/R645K/1674V, I161V/A235E/R645K/A699V, I161V/K249R/G412A/M476I/K605R/R645K/S659P/A660P/1674V, I161V/R252L/R319V/R352K/G412A/A423Q/M476I/K605R/R645K, I161V/R252L/R319V/1674V, I161V/G269R/R434K/A699V, I161V/R319V/A660P, 1161V/R352K, I161V/A423Q/R645K, I161V/K605R, S162A, S162A/R170L/A194V/A235E/K249R/G269R/1674V/A699V, S162A/R170L/G412A/R434K/M476I/K605R, S162A/A194V/K249R/R645K/S659P, S162A/A235E/G269R/A423Q/M476I/K605R/R645K/S659P, S162A/R252L/R352K/G412A/R434K/R645K/A660P/A699V, S162A/G269R/R352K/G412A/M476I/K605R, S162A/R352K, S162A/A423Q/R645K, S162A/K605R/R645K, S162L, L164C, G168V, R170L, R170L/A194V/S198G/A235E/1674V/A699V, R170L/A194V/A235E/R252L/G269R/P323S/G412A/A423Q/M476I/K605R/R645K/A660P/1674V, R170L/A194V/A235E/R319V/G412A/M476I/A699V, R170L/A194V/G269R/R319V/G412A/A423Q/M476I/K605R/R645K, R170L/A194V/R319V/R352K/M476I/A699V, R170L/A235E/K249R/R352K/R645K, R170L/A235E/G269R/R352K, R170L/A235E/G269R/R434K/1674V, R170L/A235E/R319V/R352K/G412A/R434K/M476I/R645K/A660P/1674V, R170L/A235E/R352K/1674V/A699V, R170L/A235E/R352K/A699V, R170L/A235E/G412A, R170L/A235E/R645K, R170L/A235E/A660P, R170L/K249R/R252L/G269R/R352K/G412A/A423Q/R434K/M476I/S659P, R170L/K249R/R252L/G269R/A423Q/S659P, R170L/K249R/G412A, R170L/R252L/R352K/A423Q/1674V/A699V, R170L/G269R/R352K/A423Q/M476I/K605R/S659P/A660P/A699V, R170L/G269R/M476I/R645K, R170L/G412A/A423Q/M476I/K605R/R645K/A660P/1674V, R170L/M476I/1674V, W175A, W177F, A194V, A194V/S198G/A235E/R252L/P323S/G412A/K605R/R645K, A194V/S198G/R252L/R319V/A423Q/R645K/1674V, A194V/A235E/R252L, A194V/A235E/R252L/R319V/A423Q, A194V/A235E/K605R, A194V/A235E/R645K, A194V/A235E/A699V, A194V/K249R/R252L/R645K/A699V, A194V/K249R/G412A/M476I/K605R/R645K/A699V, A194V/R252L/G269R/R319V/R434K/K605R/R645K/S659P/1674V/A699V, A194V/R252L/R319V/G412A/K605R, A194V/G269R/R319V/G412A/K605R/1674V/A699V, A194V/R319V/R352K/K605R/A660P, A194V/P323S/A699V, A194V/R352K/M476I/S659P, A194V/R352K/K605R/R645K/A699V, A194V/R434K, A194V/R645K/S659P, P197Q, S198G/1674V/A699V, P202S/R268K, K217T, K227E, A235E, A235E/K249R/R252L/P323S/R352K/M476I, A235E/K249R/R252L/G412A/K605R/R645K/A699V, A235E/K249R/R252L/A423Q/A699V, A235E/K249R/R252L/R434K/K605R/R645K/A699V, A235E/R252L, A235E/R252L/G269R/R352K/R434K/K605R/1674V/A699V, A235E/R252L/G269R/R352K/M476I/R645K/1674V, A235E/R252L/G269R/A423Q/K605R/R645K/A660P/1674V, A235E/G269R/P323S/G412A/R434K/S659P/1674V/A699V, A235E/G269R/1674V/A699V, A235E/R319V/K605R/R645K/1674V, A235E/R352K, A235E/R352K/G412A/S659P/A660P/1674V/A699V, A235E/R352K/K605R, A235E/G412A/R645K, A235E/M476I/1674V, A235E/K605R/R645K, K249R/R252L/R319V/R352K/R645K, K249R/R252L/R319V/K605R/A660P, K249R/R252L/G412A/A423Q/R645K/1674V/A699V, K249R/G412A/M476I/K605R, R252L/R352K/R645K/A660P, R252L/R645K, E253D, S255V/D260A/G398R/P557R, S255V/F287L, S255V/F287L/P391S/V472A/P557R/L658V/A660S, S255V/F287L/P391S/V472A/L658V/A660S/H685A, S255V/F287L/P391S/M476L/P557R, S255V/F287L/P391S/L658V, S255V/P391S, S255V/P391S/G398R/M476L/P557R, S255V/P391S/G398R/M476L/K602H/L658V/A660S/H685A, S255V/P391S/G398R/G513T/P557R/L658V/N743T/F758W, S255V/P391S/V472A/G513T/F758W, S255V/P391S/V472A/P557R/L658V/A660S/H685A/N743T/F758W, S255V/P391S/V472A/P557R/L658V/A660S/F758W, S255V/P391S/V472A/L658V/A660S/F758W, S255V/P391S/V472A/H685A, S255V/P391S/M476L/G513T/P557R/K602H/H685A/F758W, S255V/P391S/M476L/P557R/K602H/A660S/F758W, S255V/P391S/M476L/P557R/L658V, S255V/P391S/M476L/F758W, S255V/P391S/G513T/K602H/L658V/A660S, S255V/P391S/P557R, S255V/P391S/K602H, S255V/P391S/H685A/F758W, S255V/P391S/N743T, S255V/G398R/V472A/M476L/P557R/L658V, S255V/G398R/M476L, S255V/G398R/M476L/P557R/L658V/A660S/H685A, S255V/G398R/G513T/F758W, S255V/V472A/F758W, S255V/G513T/P557R, S255V/G513T/P557R/H685A/F758W, S255V/P557R/L658V, S255V/K602H, S255V/A660S, F267Y, R268K, G269R/R352K, G269R/R352K/G412A, G269R/K605R, R270Y, R271H/V472A, K2791, F281M, F287L/P391S/V472A, F287L/P391S/V472A/M476L/P557R/K602H/L658V/A660S/H685A/N743T, F287L/P391S/M476L/G513T, F287L/P391S/G513T/P557R/A660S/H685A/N743T/F758W, F287L/P391S/P557R, F287L/P391S/P557R/K602H/L658V/N743T, F287L/P391S/P557R/H685A, F287L/P391S/K602H, F287L/H685A, L297T, R319V, R319V/R352K/G412A/R434K/A699V, R319V/R434K/M476I, R322V, E329Q, L331S, G339E, P347I, A350F, R352G, R352K, R352K/G412A/K605R/R645K/A699V, R352K/K605R/R645K/A699V, R352K/K605R/S659P, R352K/R645K, L354I, E356N, R358N, L360F, L361I, L365I, E372N, P377Q, T379I, P391S, P391S/G398R/M476L/H685A, P391S/G398R/G513T/K602H/L658V/A660S/F758W, P391S/A451V/V472A/G513T/P557R, P391S/V472A/M476L/P557R/K602H, P391S/V472A/G513T/P557R/K602H/L658V, P391S/V472A/G513T/K602H/L658V, P391S/M476L/G513T/N743T, P391S/M476L/P557R, P391S/G513T/L658V/A660S, P391S/P557R, P391S/P557R/F758W, P391S/K602H, P391S/K602H/F758W, P391S/L658V, P391S/A660S/N743T/F758W, P391S/H685A, P396K, G398R, G398R/V472A, G398R/P557R/K602H/L658V, G398T, G412A, G412A/R645K, G412A/1674V, A423Q, A423Q/M476I/K605R, A423Q/M476I/R645K/1674V, A423Q/R645K, A423Q/A699V, A423V, L425F, L426V, L435Y, E440T, A455V, T456K, A467N, V472A, V472A/M476L/G513T/N743T, V472A/M476L/P557R/L658V/F758W, V472A/G513T/P557R/L658V/N743T/F758W, V472A/G513T/P557R/A660S/H685A, V472A/G513T/L658V/A660S/N743T, V472A/P557R/A660S/H685A/N743T/F758W, V472A/F758W, M476I, M476L, M476L/P557R/F758W, M476L/H685A, M476L/F758W, R478H/P557R, L479M, R485K, E506N, G508N, G513T, G513T/P557R/N743T, G513T/P557R/F758W, R532M, E533D, K540N, P557R, P557R/L658V/A660S, P557R/L658V/H685A, P557R/N743T, P564E, K602H, K602H/H685A, K602H/Q703R, K605R/A699V, E635K, E643H, R645K, R645K/S659P, R645K/1674V/A699V, L658V, L658V/A660S, L658V/H685A/F758W, S659E, A660S, 1674V, R704K, K713Q, K718D, F758W, and A810K, or combinations thereof, wherein the amino acid positions are relative to the reference sequence of SEQ ID NO: 2.

In some embodiments, the engineered DNA polymerase comprises at least one or more substitutions or substitution sets at amino acid positions selected from 11/287, 29, 50/255/557/685, 145/255/287/391/472/476/557, 145/255/391/472/557/685, 145/255/391/685, 145/255/472/476/557/685/743/758, 145/255/557/658/660/685, 145/391/472/476/513/557/658/685/758, 145/391/513/557/602, 145/391/557/743, 145/398/472/557/660, 145/557/658, 145/743, 161/162/269/476/660, 161/162/659/660/674, 161/170/235/269/319, 161/170/235/319/412, 161/194/235/249/352/412/434/674/699, 161/194/252/412/605, 161/235/252/319/352/645/674/699, 161/235/645/699, 161/249/412/476/605/645/659/660/674, 161/252/319/352/412/423/476/605/645, 161/319/660, 161/352, 162, 162/352, 162/423/645, 170, 170/194/235/252/269/323/412/423/476/605/645/660/674, 170/235/249/352/645, 170/235/319/352/412/434/476/645/660/674, 170/235/352/674/699, 170/235/352/699, 170/235/412, 170/235/645, 170/235/660, 170/252/352/423/674/699, 170/269/352/423/476/605/659/660/699, 194, 194/198/252/319/423/645/674, 194/235/252, 194/252/269/319/434/605/645/659/674/699, 194/269/319/412/605/674/699, 194/319/352/605/660, 194/352/605/645/699, 194/645/659, 197, 217, 227, 235/249/252/323/352/476, 235/249/252/423/699, 235/252, 235/252/269/423/605/645/660/674, 235/269/323/412/434/659/674/699, 235/352, 235/352/605, 235/605/645, 249/252/319/605/660, 252/645, 255, 255/391/472/557/660/743, 255/391/476/513/557/602/685/758, 255/391/476/557/658, 255/391/557, 255/398/472/476/557/658, 255/398/476/557/658/660/685, 255/476, 255/660, 268, 269/352, 269/352/412, 269/605, 319, 319/352/412/434/699, 319/434/476, 329, 331, 339, 352, 352/412/605/645/699, 354, 358, 360, 391, 391/398/476/685, 391/451/472/513/557, 391/472/476/557/602, 391/472/476/557/658/660/743/758, 391/557, 391/581, 412, 412/645, 412/674, 423/476/645/674, 423/645, 467, 478/557, 506, 540, 557, 557/658/660, 557/658/685, 602/701, 602/703, 605/699, 658, 658/660, 660, 699, 713, and 743, wherein the amino acid positions are relative to the reference sequence of SEQ ID NO: 2.

In some embodiments, the engineered DNA polymerase comprises one or more substitutions or substitution sets selected from 11D/287L, 29N, 50H/255V/557R/685A, 145H/255V/287L/391S/472A/476L/557R, 145H/255V/391S/472A/557R/685A, 145H/255V/391S/685A, 145H/255V/472A/476L/557R/685A/743T/758W, 145H/255V/557R/658V/660S/685A, 145H/391S/472A/476L/513T/557R/658V/685A/758W, 145H/391S/513T/557R/602H, 145H/391S/557R/743T, 145H/398R/472A/557R/660S, 145H/557R/658V, 145H/743T, 161V/162A/269R/476I/660P, 161V/162A/659P/660P/674V, 161V/170L/235E/269R/319V, 161V/170L/235E/319V/412A, 161V/194V/235E/249R/352K/412A/434K/674V/699V, 161V/194V/252L/412A/605R, 161V/235E/252L/319V/352K/645K/674V/699V, 161V/235E/645K/699V, 161V/249R/412A/476I/605R/645K/659P/660P/674V, 161V/252L/319V/352K/412A/423Q/4761/605R/645K, 161V/319V/660P, 161V/352K, 162A, 162A/352K, 162A/423Q/645K, 170L, 170L/194V/235E/252L/269R/323S/412A/423Q/476I/605R/645K/660P/674V, 170L/235E/249R/352K/645K, 170L/235E/319V/352K/412A/434K/476I/645K/660P/674V, 170L/235E/352K/674V/699V, 170L/235E/352K/699V, 170L/235E/412A, 170L/235E/645K, 170L/235E/660P, 170L/252L/352K/423Q/674V/699V, 170L/269R/352K/423Q/476I/605R/659P/660P/699V, 194V, 194V/198G/252L/319V/423Q/645K/674V, 194V/235E/252L, 194V/252L/269R/319V/434K/605R/645K/659P/674V/699V, 194V/269R/319V/412A/605R/674V/699V, 194V/319V/352K/605R/660P, 194V/352K/605R/645K/699V, 194V/645K/659P, 197Q, 217T, 227E, 235E/249R/252L/323S/352K/476I, 235E/249R/252L/423Q/699V, 235E/252L, 235E/252L/269R/423Q/605R/645K/660P/674V, 235E/269R/323S/412A/434K/659P/674V/699V, 235E/352K, 235E/352K/605R, 235E/605R/645K, 249R/252L/319V/605R/660P, 252L/645K, 255V, 255V/391S/472A/557R/660S/743T, 255V/391S/476L/513T/557R/602H/685A/758W, 255V/391S/476L/557R/658V, 255V/391S/557R, 255V/398R/472A/476L/557R/658V, 255V/398R/476L/557R/658V/660S/685A, 255V/476L, 255V/660S, 268K, 269R/352K, 269R/352K/412A, 269R/605R, 319V, 319V/352K/412A/434K/699V, 319V/434K/4761, 329Q, 331S, 339E, 352G, 352K, 352K/412A/605R/645K/699V, 3541, 358N, 360F, 391S, 391S/398R/476L/685A, 391S/451V/472A/513T/557R, 391S/472A/476L/557R/602H, 391S/472A/476L/557R/658V/660S/743T/758W, 391S/557R, 391S/581D, 412A, 412A/645K, 412A/674V, 423Q/4761/645K/674V, 423Q/645K, 467N, 478H/557R, 506N, 540N, 557R, 557R/658V/660S, 557R/658V/685A, 602H/701L, 602H/703R, 605R/699V, 658V, 658V/660S, 660S, 699V, 713Q, and 743T, wherein the amino acid positions are relative to the reference sequence of SEQ ID NO: 2.

In some embodiments, the engineered DNA polymerase comprises one or more substitutions or substitution sets selected from G11D/F287L, H29N, Q50H/S255V/P557R/H685A, Q145H/S255V/F287L/P391S/V472A/M476L/P557R, Q145H/S255V/P391S/V472A/P557R/H685A, Q145H/S255V/P391S/H685A, Q145H/S255V/V472A/M476L/P557R/H685A/N743T/F758W, Q145H/S255V/P557R/L658V/A660S/H685A, Q145H/P391S/V472A/M476L/G513T/P557R/L658V/H685A/F758W, Q145H/P391S/G513T/P557R/K602H, Q145H/P391S/P557R/N743T, Q145H/G398R/V472A/P557R/A660S, Q145H/P557R/L658V, Q145H/N743T, I161V/S162A/G269R/M476I/A660P, I161V/S162A/S659P/A660P/1674V, I161V/R170L/A235E/G269R/R319V, I161V/R170L/A235E/R319V/G412A, I161V/A194V/A235E/K249R/R352K/G412A/R434K/1674V/A699V, I161V/A194V/R252L/G412A/K605R, I161V/A235E/R252L/R319V/R352K/R645K/1674V/A699V, I161V/A235E/R645K/A699V, I161V/K249R/G412A/M476I/K605R/R645K/S659P/A660P/1674V, I161V/R252L/R319V/R352K/G412A/A423Q/M476I/K605R/R645K, I161V/R319V/A660P, I161V/R352K, S162A, S162A/R352K, S162A/A423Q/R645K, R170L, R170L/A194V/A235E/R252L/G269R/P323S/G412A/A423Q/M476I/K605R/R645K/A660P/1674V, R170L/A235E/K249R/R352K/R645K, R170L/A235E/R319V/R352K/G412A/R434K/M476I/R645K/A660P/1674V, R170L/A235E/R352K/1674V/A699V, R170L/A235E/R352K/A699V, R170L/A235E/G412A, R170L/A235E/R645K, R170L/A235E/A660P, R170L/R252L/R352K/A423Q/1674V/A699V, R170L/G269R/R352K/A423Q/M476I/K605R/S659P/A660P/A699V, A194V, A194V/S198G/R252L/R319V/A423Q/R645K/1674V, A194V/A235E/R252L, A194V/R252L/G269R/R319V/R434K/K605R/R645K/S659P/1674V/A699V, A194V/G269R/R319V/G412A/K605R/1674V/A699V, A194V/R319V/R352K/K605R/A660P, A194V/R352K/K605R/R645K/A699V, A194V/R645K/S659P, P197Q, K217T, K227E, A235E/K249R/R252L/P323S/R352K/M476I, A235E/K249R/R252L/A423Q/A699V, A235E/R252L, A235E/R252L/G269R/A423Q/K605R/R645K/A660P/1674V, A235E/G269R/P323S/G412A/R434K/S659P/1674V/A699V, A235E/R352K, A235E/R352K/K605R, A235E/K605R/R645K, K249R/R252L/R319V/K605R/A660P, R252L/R645K, S255V, S255V/P391S/V472A/P557R/A660S/N743T, S255V/P391S/M476L/G513T/P557R/K602H/H685A/F758W, S255V/P391S/M476L/P557R/L658V, S255V/P391S/P557R, S255V/G398R/V472A/M476L/P557R/L658V, S255V/G398R/M476L/P557R/L658V/A660S/H685A, S255V/M476L, S255V/A660S, R268K, G269R/R352K, G269R/R352K/G412A, G269R/K605R, R319V, R319V/R352K/G412A/R434K/A699V, R319V/R434K/M476I, E329Q, L331S, G339E, R352G, R352K, R352K/G412A/K605R/R645K/A699V, L354I, R358N, L360F, P391S, P391S/G398R/M476L/H685A, P391S/A451V/V472A/G513T/P557R, P391S/V472A/M476L/P557R/K602H, P391S/V472A/M476L/P557R/L658V/A660S/N743T/F758W, P391S/P557R, P391S/G581D, G412A, G412A/R645K, G412A/1674V, A423Q/M476I/R645K/1674V, A423Q/R645K, A467N, R478H/P557R, E506N, K540N, P557R, P557R/L658V/A660S, P557R/L658V/H685A, K602H/F701L, K602H/Q703R, K605R/A699V, L658V, L658V/A660S, A660S, A699V, K713Q, and N743T, wherein the amino acid positions are relative to the reference sequence of SEQ ID NO: 2.

In some additional embodiments, the present invention provides an engineered DNA polymerase or a functional fragment thereof, comprising a polypeptide sequence having at least 75% or more sequence identity to a reference sequence comprising residues 12 to 841 of SEQ ID NO: 178, or to a reference sequence of SEQ ID NO: 178, wherein the polypeptide sequence comprises one or more substitutions relative to the reference sequence comprising residues 12 to 841 of SEQ ID NO: 178, or to the reference sequence of SEQ ID NO: 178.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 178 or to the reference sequence of SEQ ID NO: 178.

In some embodiments, the engineered DNA polymerase comprises one or more substitutions or substitution sets selected from 12, 14, 15, 17, 19, 36, 39, 44, 45, 82, 86, 87, 95, 122, 139, 139/395, 159, 167, 170/194, 170/194/423/451/557/658/660/743, 170/194/423/476/743, 170/194/423/557/658/659/743, 170/194/423/557/658/660/685, 170/194/423/557/658/743, 170/194/451/476/557/658, 170/194/451/476/557/658/659/660/743, 170/194/451/476/557/658/685, 170/194/451/557/659/660, 170/194/476/557/659, 170/194/557, 170/194/658, 170/194/658/659/660/685/743, 170/194/658/743, 170/423, 170/423/451/476/557/658/659/743, 170/423/451/557, 170/423/451/557/658/659/685/743, 170/423/451/557/659, 170/423/476, 170/423/476/658/659, 170/423/476/658/659/660/685/743, 170/423/476/658/660/685/743, 170/423/476/658/685, 170/423/476/658/685/743, 170/423/476/685, 170/423/476/685/743, 170/423/557/658/659, 170/423/557/658/660/743, 170/423/557/658/743, 170/423/557/659/660/685/743, 170/423/557/660/743, 170/423/557/685, 170/423/557/685/743, 170/423/658, 170/423/658/659/743, 170/423/658/660, 170/423/743, 170/451/557, 170/451/557/658/660, 170/476/557/658/659, 170/476/557/658/685, 170/476/557/743, 170/476/659, 170/557, 170/557/658/660/685/687, 170/658, 170/658/659, 170/658/659/743, 170/659, 170/660, 179, 194/423/451/557/658/659/743, 194/423/476/658/660/685, 194/423/557/658/659/743, 194/423/685, 194/451/476/557/658/659, 194/451/476/557/658/659/660/743, 194/557, 194/557/658/659/685, 194/557/659/743, 194/658/659/685, 194/658/660, 197, 198, 198/423/476/557/743, 205, 209, 214, 222, 228, 249, 250/269/331, 252, 252/258/331, 252/258/331/352/467/506/674, 252/269, 252/269/331/339/412/413, 252/269/331/352/412/674/704, 252/269/331/506/674, 252/269/339/352/356/660/704, 252/269/339/352/412/699/704, 252/269/352/660, 252/269/412/674, 252/269/413/467/660/699/704, 252/269/467/506, 252/269/674, 252/331, 252/331/339/352/467/506, 252/331/339/412/413/467/699, 252/331/352/412/413/467/660/704, 252/331/352/412/506/674, 252/331/358/506, 252/331/412/413, 252/331/412/413/674, 252/331/412/467/506/699/704, 252/331/412/506/674, 252/331/412/506/704, 252/331/413, 252/331/467/660/674, 252/331/660, 252/331/699, 252/339/352/467/674/699, 252/339/352/674/704, 252/339/412/413/467/674, 252/339/412/413/674/699/704, 252/339/412/467/674, 252/339/412/674/704, 252/339/467/506/699/704, 252/341/412/413, 252/341/412/674, 252/352/412, 252/352/506, 252/352/660, 252/358/674, 252/412, 252/412/413, 252/412/467, 252/412/467/660/699/704, 252/413, 252/413/506/660/704, 252/413/699, 252/467, 252/467/506/674/704, 252/506/704, 252/674, 252/699, 252/704, 264, 268, 268/510, 268/621, 269, 269/331/352/412/467/506/704, 269/331/352/467/506/699, 269/331/352/467/699, 269/331/412/413/467/506/660/674, 269/331/413/506, 269/331/467/506/660, 269/331/660/699, 269/331/674/699, 269/331/699, 269/331/699/704, 269/339/506/704, 269/352/467/506/674, 269/358, 269/358/412/660/704, 269/358/467/506/660/699, 269/412/413/506/704, 269/412/413/674, 269/412/467/704, 269/467/506, 269/506, 269/599/660/674/699, 269/674, 269/699/704, 269/704, 272/352/506, 275, 301, 323, 331, 331/339/412/413/467/506/674/704, 331/339/412/467/506/704, 331/352/358/699/704, 331/352/412/413/506, 331/358, 331/358/467/674/699/704, 331/412, 331/412/413/467/699/704, 331/412/467/674/704, 331/412/467/704, 331/412/660/699, 331/413/467/660/674, 331/467, 331/467/506, 331/467/674/699, 331/506, 331/506/674/699, 331/660/674/699, 331/674, 331/674/699, 331/699, 331/704, 339, 339/352, 339/352/412/419/467/506/660/674/699, 339/352/412/467/660, 339/352/412/660/674, 339/352/674/699, 339/412/413, 339/412/413/467/674/699, 339/412/467/674, 339/467/674, 339/506, 339/506/699, 339/506/704, 339/660, 339/660/674, 341/352/704, 341/467, 341/467/506/660/674, 341/467/674, 345, 352, 352/358, 352/412/413/467/506/699, 352/413/467/506, 352/413/674, 352/660, 352/674, 355, 358/412, 358/413, 358/674, 412, 412/413/467/660, 412/413/467/674, 412/467/506/674/704, 412/467/660/674/699, 412/660, 412/674, 412/674/699, 413/467, 413/506, 413/506/704, 413/674/699/704/740, 423, 423/451/476/557/658/685, 423/451/476/658/685, 423/451/557, 423/451/557/658, 423/451/557/658/685, 423/476, 423/476/557/658/659/660, 423/476/557/658/659/685/743, 423/476/557/659/660/685/743, 423/476/557/659/660/743, 423/476/658/660/743, 423/476/658/685/743, 423/476/658/743, 423/557/658, 423/557/658/659/660, 423/557/658/659/660/685, 423/557/658/660/743, 423/557/685, 423/557/685/743, 423/658, 423/658/659/660, 423/658/660, 423/660, 423/685, 423/685/743, 423/743, 428, 433, 433/497, 467, 467/506/699/704, 467/658/660/674/699, 470, 476, 476/480/557/659, 476/557/658/659, 476/557/658/660/685, 476/557/659, 476/557/659/660, 476/658/659/660/685/743, 476/658/660/743, 477, 478, 485, 489/557/743, 492, 501, 506/660, 506/674/699/704, 506/674/704, 511, 514, 517, 520, 525/557/659/660/743, 533, 534, 534/670, 549, 557, 557/658, 557/658/659, 557/658/659/660, 557/659/660, 557/660, 557/743, 565, 596, 598, 642, 658, 658/659, 658/659/660/685/743, 658/659/685, 658/743, 659/660, 659/660/685/743, 674, 674/699, 685, 689, 695, 696, 699, 699/704, 704, 707, 725, 743, 745, 747, 783, and 821, or combinations thereof, wherein the amino acid positions are relative to the reference sequence of SEQ ID NO: 178.

In some embodiments, the engineered DNA polymerase comprises one or more substitutions or substitution sets are selected from 12E, 12L, 12S, 12V, 14E, 14V, 15V, 17S, 19A, 19S, 36H, 39E, 39P, 39V, 44A, 44L, 45L, 45S, 82K, 86G, 87A, 87P, 87R, 95D, 95G, 95R, 122A, 122Q, 139D/395A, 139S, 159Q, 167D, 170L/194V, 170L/194V/423Q/451H/557R/658V/660S/743T, 170L/194V/423Q/476L/743T, 170L/194V/423Q/557R/658V/659P/743T, 170L/194V/423Q/557R/658V/660S/685A, 170L/194V/423Q/557R/658V/743T, 170L/194V/451H/476L/557R/658V, 170L/194V/451H/476L/557R/658V/659P/660S/743T, 170L/194V/451H/476L/557R/658V/685A, 170L/194V/451H/557R/659P/660S, 170L/194V/476L/557R/659P, 170L/194V/557R, 170L/194V/658V, 170L/194V/658V/659P/660S/685A/743T, 170L/194V/658V/743T, 170L/423Q, 170L/423Q/451H/476L/557R/658V/659P/743T, 170L/423Q/451H/557R, 170L/423Q/451H/557R/658V/659P/685A/743T, 170L/423Q/451H/557R/659P, 170L/423Q/476L, 170L/423Q/476L/658V/659P, 170L/423Q/476L/658V/659P/660S/685A/743T, 170L/423Q/476L/658V/660S/685A/743T, 170L/423Q/476L/658V/685A, 170L/423Q/476L/658V/685A/743T, 170L/423Q/476L/685A, 170L/423Q/476L/685A/743T, 170L/423Q/557R/658V/659P, 170L/423Q/557R/658V/660S/743T, 170L/423Q/557R/658V/743T, 170L/423Q/557R/659P/660S/685A/743T, 170L/423Q/557R/660S/743T, 170L/423Q/557R/685A, 170L/423Q/557R/685A/743T, 170L/423Q/658V, 170L/423Q/658V/659P/743T, 170L/423Q/658V/660S, 170L/423Q/743T, 170L/451H/557R, 170L/451H/557R/658V/660S, 170L/476L/557R/658V/659P, 170L/476L/557R/658V/685A, 170L/476L/557R/743T, 170L/476L/659P, 170L/557R, 170L/557R/658V/660S/685A/687F, 170L/658V, 170L/658V/659P, 170L/658V/659P/743T, 170L/659P, 170L/660S, 179M, 194V/423Q/451H/557R/658V/659P/743T, 194V/423Q/476L/658V/660S/685A, 194V/423Q/557R/658V/659P/743T, 194V/423Q/685A, 194V/451H/476L/557R/658V/659P, 194V/451H/476L/557R/658V/659P/660S/743T, 194V/557R, 194V/557R/658V/659P/685A, 194V/557R/659P/743T, 194V/658V/659P/685A, 194V/658V/660S, 197K, 198G, 198G/423Q/476L/557R/743T, 198H, 198P, 198V, 205R, 205S, 209P, 209R, 209S, 209V, 209W, 214D, 214R, 2221, 228S, 249E, 250I/269R/331S, 252L, 252L/258H/331S, 252L/258H/331S/352K/467N/506N/674V, 252L/269R, 252L/269R/331S/339E/412A/413K, 252L/269R/331S/352K/412A/674V/704K, 252L/269R/331S/506N/674V, 252L/269R/339E/352K/356G/660P/704K, 252L/269R/339E/352K/412A/699V/704K, 252L/269R/352K/660P, 252L/269R/412A/674V, 252L/269R/413K/467N/660P/699V/704K, 252L/269R/467N/506N, 252L/269R/674V, 252L/331S, 252L/331S/339E/352K/467N/506N, 252L/331S/339E/412A/413K/467N/699V, 252L/331S/352K/412A/413K/467N/660P/704K, 252L/331S/352K/412A/506N/674V, 252L/331S/358N/506N, 252L/331S/412A/413K, 252L/331S/412A/413K/674V, 252L/331S/412A/467N/506N/699V/704K, 252L/331S/412A/506N/674V, 252L/331S/412A/506N/704K, 252L/331S/413K, 252L/331S/467N/660P/674V, 252L/331S/660P, 252L/331S/699V, 252L/339E/352K/467N/674V/699V, 252L/339E/352K/674V/704K, 252L/339E/412A/413K/467N/674V, 252L/339E/412A/413K/674V/699V/704K, 252L/339E/412A/467N/674V, 252L/339E/412A/674V/704K, 252L/339E/467N/506N/699V/704K, 252L/341A/412A/413K, 252L/341A/412A/674V, 252L/352K/412A, 252L/352K/506N, 252L/352K/660P, 252L/358N/674V, 252L/412A, 252L/412A/413K, 252L/412A/467N, 252L/412A/467N/660P/699V/704K, 252L/413K, 252L/413K/506N/660P/704K, 252L/413K/699V, 252L/467N, 252L/467N/506N/674V/704K, 252L/506N/704K, 252L/674V, 252L/699V, 252L/704K, 264A, 264S, 268A/621N, 268G, 268H/510A, 269R, 269R/331S/352K/412A/467N/506N/704K, 269R/331S/352K/467N/506N/699V, 269R/331S/352K/467N/699V, 269R/331S/412A/413K/467N/506N/660P/674V, 269R/331S/413K/506N, 269R/331S/467N/506N/660P, 269R/331S/660P/699V, 269R/331S/674V/699V, 269R/331S/699V, 269R/331S/699V/704K, 269R/339E/506N/704K, 269R/352K/467N/506N/674V, 269R/358N, 269R/358N/412A/660P/704K, 269R/358N/467N/506N/660P/699V, 269R/412A/413K/506N/704K, 269R/412A/413K/674V, 269R/412A/467N/704K, 269R/467N/506N, 269R/506N, 269R/599F/660P/674V/699V, 269R/674V, 269R/699V/704K, 269R/704K, 272S/352K/506N, 275E, 275F, 275L, 275M, 301G, 301M, 301T, 323L, 323Q, 331S, 331S/339E/412A/413K/467N/506N/674V/704K, 331S/339E/412A/467N/506N/704K, 331S/352K/358N/699V/704K, 331S/352K/412A/413K/506N, 331S/358N, 331S/358N/467N/674V/699V/704K, 331S/412A, 331S/412A/413K/467N/699V/704K, 331S/412A/467N/674V/704K, 331S/412A/467N/704K, 331S/412A/660P/699V, 331S/413K/467N/660P/674V, 331S/467N, 331S/467N/506N, 331S/467N/674V/699V, 331S/506N, 331S/506N/674V/699V, 331S/660P/674V/699V, 331S/674V, 331S/674V/699V, 331S/699V, 331S/704K, 339E, 339E/352K, 339E/352K/412A/419K/467N/506N/660P/674V/699V, 339E/352K/412A/467N/660P, 339E/352K/412A/660P/674V, 339E/352K/674V/699V, 339E/412A/413K, 339E/412A/413K/467N/674V/699V, 339E/412A/467N/674V, 339E/467N/674V, 339E/506N, 339E/506N/699V, 339E/506N/704K, 339E/660P, 339E/660P/674V, 341A/352K/704K, 341A/467N, 341A/467N/506N/660P/674V, 341A/467N/674V, 345P, 352G, 352K/358N, 352K/412A/413K/467N/506N/699V, 352K/413K/467N/506N, 352K/413K/674V, 352K/660P, 352K/674V, 355G, 358N/412A, 358N/413K, 358N/674V, 412A, 412A/413K/467N/660P, 412A/413K/467N/674V, 412A/467N/506N/674V/704K, 412A/467N/660P/674V/699V, 412A/660P, 412A/674V, 412A/674V/699V, 413K/467N, 413K/506N, 413K/506N/704K, 413K/674V/699V/704K/740H, 423Q, 423Q/451H/476L/557R/658V/685A, 423Q/451H/476L/658V/685A, 423Q/451H/557R, 423Q/451H/557R/658V, 423Q/451H/557R/658V/685A, 423Q/476L, 423Q/476L/557R/658V/659P/660S, 423Q/476L/557R/658V/659P/685A/743T, 423Q/476L/557R/659P/660S/685A/743T, 423Q/476L/557R/659P/660S/743T, 423Q/476L/658V/660S/743T, 423Q/476L/658V/685A/743T, 423Q/476L/658V/743T, 423Q/557R/658V, 423Q/557R/658V/659P/660S, 423Q/557R/658V/659P/660S/685A, 423Q/557R/658V/660S/743T, 423Q/557R/685A, 423Q/557R/685A/743T, 423Q/658V, 423Q/658V/659P/660S, 423Q/658V/660S, 423Q/660S, 423Q/685A, 423Q/685A/743T, 423Q/743T, 428C, 428G, 428Q, 433G, 433K, 433P/497N, 433R, 433S, 433V, 467N, 467N/506N/699V/704K, 467N/658V/660S/674V/699V, 470E, 470G, 470H, 470V, 476L, 476L/480K/557R/659P, 476L/557R/658V/659P, 476L/557R/658V/660S/685A, 476L/557R/659P, 476L/557R/659P/660S, 476L/658V/659P/660S/685A/743T, 476L/658V/660S/743T, 477E, 477T, 478G, 478I, 478L, 478S, 485A, 485E, 485G, 485I, 485M, 485T, 485V, 489Q/557R/743T, 492M, 492R, 492Y, 501K, 501L, 506N/660P, 506N/674V/699V/704K, 506N/674V/704K, 511L, 514G, 514V, 517A, 517H, 517L, 517M, 517R, 517T, 520V, 525V/557R/659P/660S/743T, 533L, 533P, 534A, 534A/670V, 549V, 557R, 557R/658V, 557R/658V/659P, 557R/658V/659P/660S, 557R/659P/660S, 557R/660S, 557R/743T, 565R, 565S, 565Y, 596S, 598G, 598H, 598K, 598L, 598R, 642E, 658V, 658V/659P, 658V/659P/660S/685A/743T, 658V/659P/685A, 658V/743T, 659P/660S, 659P/660S/685A/743T, 674V, 674V/699V, 685A, 689D, 689N, 695K, 695Q, 695T, 696A, 696G, 696K, 696Q, 699V, 699V/704K, 704K, 707A, 707R, 707S, 725H, 743T, 745P, 747V, 783H, and 821S, or combinations thereof, wherein the amino acid positions are relative to the reference sequence of SEQ ID NO: 178.

In some embodiments, the engineered DNA polymerase comprises one or more substitutions or substitution sets selected from M12E, M12L, M12S, M12V, P14E, P14V, L15V, E17S, K19A, K19S, F36H, K39E, K39P, K39V, S44A, S44L, R45L, R45S, R82K, Y86G, E87A, E87P, E87R, P95D, P95G, P95R, P122A, P122Q, R139D/T395A, R139S, P159Q, E167D, R170L/A194V, R170L/A194V/A423Q/A451H/P557R/L658V/A660S/N743T, R170L/A194V/A423Q/M476L/N743T, R170L/A194V/A423Q/P557R/L658V/S659P/N743T, R170L/A194V/A423Q/P557R/L658V/A660S/H685A, R170L/A194V/A423Q/P557R/L658V/N743T, R170L/A194V/A451H/M476L/P557R/L658V, R170L/A194V/A451H/M476L/P557R/L658V/S659P/A660S/N743T, R170L/A194V/A451H/M476L/P557R/L658V/H685A, R170L/A194V/A451H/P557R/S659P/A660S, R170L/A194V/M476L/P557R/S659P, R170L/A194V/P557R, R170L/A194V/L658V, R170L/A194V/L658V/S659P/A660S/H685A/N743T, R170L/A194V/L658V/N743T, R170L/A423Q, R170L/A423Q/A451H/M476L/P557R/L658V/S659P/N743T, R170L/A423Q/A451H/P557R, R170L/A423Q/A451H/P557R/L658V/S659P/H685A/N743T, R170L/A423Q/A451H/P557R/S659P, R170L/A423Q/M476L, R170L/A423Q/M476L/L658V/S659P, R170L/A423Q/M476L/L658V/S659P/A660S/H685A/N743T, R170L/A423Q/M476L/L658V/A660S/H685A/N743T, R170L/A423Q/M476L/L658V/H685A, R170L/A423Q/M476L/L658V/H685A/N743T, R170L/A423Q/M476L/H685A, R170L/A423Q/M476L/H685A/N743T, R170L/A423Q/P557R/L658V/S659P, R170L/A423Q/P557R/L658V/A660S/N743T, R170L/A423Q/P557R/L658V/N743T, R170L/A423Q/P557R/S659P/A660S/H685A/N743T, R170L/A423Q/P557R/A660S/N743T, R170L/A423Q/P557R/H685A, R170L/A423Q/P557R/H685A/N743T, R170L/A423Q/L658V, R170L/A423Q/L658V/S659P/N743T, R170L/A423Q/L658V/A660S, R170L/A423Q/N743T, R170L/A451H/P557R, R170L/A451H/P557R/L658V/A660S, R170L/M476L/P557R/L658V/S659P, R170L/M476L/P557R/L658V/H685A, R170L/M476L/P557R/N743T, R170L/M476L/S659P, R170L/P557R, R170L/P557R/L658V/A660S/H685A/L687F, R170L/L658V, R170L/L658V/S659P, R170L/L658V/S659P/N743T, R170L/S659P, R170L/A660S, K179M, A194V/A423Q/A451H/P557R/L658V/S659P/N743T, A194V/A423Q/M476L/L658V/A660S/H685A, A194V/A423Q/P557R/L658V/S659P/N743T, A194V/A423Q/H685A, A194V/A451H/M476L/P557R/L658V/S659P, A194V/A451H/M476L/P557R/L658V/S659P/A660S/N743T, A194V/P557R, A194V/P557R/L658V/S659P/H685A, A194V/P557R/S659P/N743T, A194V/L658V/S659P/H685A, A194V/L658V/A660S, P197K, S198G, S198G/A423Q/M476L/P557R/N743T, S198H, S198P, S198V, K205R, K205S, E209P, E209R, E209S, E209V, E209W, K214D, K214R, L222I, N228S, K249E, L250I/G269R/L331S, R252L, R252L/R258H/L331S, R252L/R258H/L331S/R352K/A467N/E506N/1674V, R252L/G269R, R252L/G269R/L331S/G339E/G412A/E413K, R252L/G269R/L331S/R352K/G412A/1674V/R704K, R252L/G269R/L331S/E506N/1674V, R252L/G269R/G339E/R352K/E356G/A660P/R704K, R252L/G269R/G339E/R352K/G412A/A699V/R704K, R252L/G269R/R352K/A660P, R252L/G269R/G412A/I674V, R252L/G269R/E413K/A467N/A660P/A699V/R704K, R252L/G269R/A467N/E506N, R252L/G269R/1674V, R252L/L331S, R252L/L331S/G339E/R352K/A467N/E506N, R252L/L331S/G339E/G412A/E413K/A467N/A699V, R252L/L331S/R352K/G412A/E413K/A467N/A660P/R704K, R252L/L331S/R352K/G412A/E506N/1674V, R252L/L331S/R358N/E506N, R252L/L331S/G412A/E413K, R252L/L331S/G412A/E413K/1674V, R252L/L331S/G412A/A467N/E506N/A699V/R704K, R252L/L331S/G412A/E506N/1674V, R252L/L331S/G412A/E506N/R704K, R252L/L331S/E413K, R252L/L331S/A467N/A660P/1674V, R252L/L331S/A660P, R252L/L331S/A699V, R252L/G339E/R352K/A467N/1674V/A699V, R252L/G339E/R352K/1674V/R704K, R252L/G339E/G412A/E413K/A467N/1674V, R252L/G339E/G412A/E413K/1674V/A699V/R704K, R252L/G339E/G412A/A467N/1674V, R252L/G339E/G412A/1674V/R704K, R252L/G339E/A467N/E506N/A699V/R704K, R252L/V341A/G412A/E413K, R252L/V341A/G412A/1674V, R252L/R352K/G412A, R252L/R352K/E506N, R252L/R352K/A660P, R252L/R358N/1674V, R252L/G412A, R252L/G412A/E413K, R252L/G412A/A467N, R252L/G412A/A467N/A660P/A699V/R704K, R252L/E413K, R252L/E413K/E506N/A660P/R704K, R252L/E413K/A699V, R252L/A467N, R252L/A467N/E506N/1674V/R704K, R252L/E506N/R704K, R252L/1674V, R252L/A699V, R252L/R704K, E264A, E264S, R268A/S621N, R268G, R268H/P510A, G269R, G269R/L331S/R352K/G412A/A467N/E506N/R704K, G269R/L331S/R352K/A467N/E506N/A699V, G269R/L331S/R352K/A467N/A699V, G269R/L331S/G412A/E413K/A467N/E506N/A660P/1674V, G269R/L331S/E413K/E506N, G269R/L331S/A467N/E506N/A660P, G269R/L331S/A660P/A699V, G269R/L331S/1674V/A699V, G269R/L331S/A699V, G269R/L331S/A699V/R704K, G269R/G339E/E506N/R704K, G269R/R352K/A467N/E506N/1674V, G269R/R358N, G269R/R358N/G412A/A660P/R704K, G269R/R358N/A467N/E506N/A660P/A699V, G269R/G412A/E413K/E506N/R704K, G269R/G412A/E413K/1674V, G269R/G412A/A467N/R704K, G269R/A467N/E506N, G269R/E506N, G269R/L599F/A660P/1674V/A699V, G269R/1674V, G269R/A699V/R704K, G269R/R704K, E272S/R352K/E506N, W275E, W275F, W275L, W275M, L301G, L301M, L301T, P323L, P323Q, L331S, L331S/G339E/G412A/E413K/A467N/E506N/1674V/R704K, L331S/G339E/G412A/A467N/E506N/R704K, L331S/R352K/R358N/A699V/R704K, L331S/R352K/G412A/E413K/E506N, L331S/R358N, L331S/R358N/A467N/1674V/A699V/R704K, L331S/G412A, L331S/G412A/E413K/A467N/A699V/R704K, L331S/G412A/A467N/1674V/R704K, L331S/G412A/A467N/R704K, L331S/G412A/A660P/A699V, L331S/E413K/A467N/A660P/1674V, L331S/A467N, L331S/A467N/E506N, L331S/A467N/1674V/A699V, L331S/E506N, L331S/E506N/1674V/A699V, L331S/A660P/1674V/A699V, L331S/1674V, L331S/1674V/A699V, L331S/A699V, L331S/R704K, G339E, G339E/R352K, G339E/R352K/G412A/E419K/A467N/E506N/A660P/1674V/A699V, G339E/R352K/G412A/A467N/A660P, G339E/R352K/G412A/A660P/1674V, G339E/R352K/I674V/A699V, G339E/G412A/E413K, G339E/G412A/E413K/A467N/1674V/A699V, G339E/G412A/A467N/1674V, G339E/A467N/1674V, G339E/E506N, G339E/E506N/A699V, G339E/E506N/R704K, G339E/A660P, G339E/A660P/1674V, V341A/R352K/R704K, V341A/A467N, V341A/A467N/E506N/A660P/1674V, V341A/A467N/1674V, E345P, R352G, R352K/R358N, R352K/G412A/E413K/A467N/E506N/A699V, R352K/E413K/A467N/E506N, R352K/E413K/1674V, R352K/A660P, R352K/1674V, K355G, R358N/G412A, R358N/E413K, R358N/1674V, G412A, G412A/E413K/A467N/A660P, G412A/E413K/A467N/1674V, G412A/A467N/E506N/1674V/R704K, G412A/A467N/A660P/1674V/A699V, G412A/A660P, G412A/1674V, G412A/1674V/A699V, E413K/A467N, E413K/E506N, E413K/E506N/R704K, E413K/I674V/A699V/R704K/P740H, A423Q, A423Q/A451H/M476L/P557R/L658V/H685A, A423Q/A451H/M476L/L658V/H685A, A423Q/A451H/P557R, A423Q/A451H/P557R/L658V, A423Q/A451H/P557R/L658V/H685A, A423Q/M476L, A423Q/M476L/P557R/L658V/S659P/A660S, A423Q/M476L/P557R/L658V/S659P/H685A/N743T, A423Q/M476L/P557R/S659P/A660S/H685A/N743T, A423Q/M476L/P557R/S659P/A660S/N743T, A423Q/M476L/L658V/A660S/N743T, A423Q/M476L/L658V/H685A/N743T, A423Q/M476L/L658V/N743T, A423Q/P557R/L658V, A423Q/P557R/L658V/S659P/A660S, A423Q/P557R/L658V/S659P/A660S/H685A, A423Q/P557R/L658V/A660S/N743T, A423Q/P557R/H685A, A423Q/P557R/H685A/N743T, A423Q/L658V, A423Q/L658V/S659P/A660S, A423Q/L658V/A660S, A423Q/A660S, A423Q/H685A, A423Q/H685A/N743T, A423Q/N743T, R428C, R428G, R428Q, E433G, E433K, E433P/D497N, E433R, E433S, E433V, A467N, A467N/E506N/A699V/R704K, A467N/L658V/A660S/1674V/A699V, L470E, L470G, L470H, L470V, M476L, M476L/E480K/P557R/S659P, M476L/P557R/L658V/S659P, M476L/P557R/L658V/A660S/H685A, M476L/P557R/S659P, M476L/P557R/S659P/A660S, M476L/L658V/S659P/A660S/H685A/N743T, M476L/L658V/A660S/N743T, R477E, R477T, R478G, R478I, R478L, R478S, R485A, R485E, R485G, R485I, R485M, R485T, R485V, H489Q/P557R/N743T, N492M, N492R, N492Y, R501K, R501L, E506N/A660P, E506N/1674V/A699V/R704K, E506N/1674V/R704K, P511L, K514G, K514V, K517A, K517H, K517L, K517M, K517R, K517T, K520V, A525V/P557R/S659P/A660S/N743T, E533L, E533P, V534A, V534A/A670V, K549V, P557R, P557R/L658V, P557R/L658V/S659P, P557R/L658V/S659P/A660S, P557R/S659P/A660S, P557R/A660S, P557R/N743T, K565R, K565S, K565Y, R596S, P598G, P598H, P598K, P598L, P598R, Q642E, L658V, L658V/S659P, L658V/S659P/A660S/H685A/N743T, L658V/S659P/H685A, L658V/N743T, S659P/A660S, S659P/A660S/H685A/N743T, 1674V, 1674V/A699V, H685A, Q689D, Q689N, Y695K, Y695Q, Y695T, E696A, E696G, E696K, E696Q, A699V, A699V/R704K, R704K, Q707A, Q707R, Q707S, T725H, N743T, R745P, K747V, E783H, and P821S, or combinations thereof, wherein the amino acid positions are relative to the reference sequence of SEQ ID NO: 178.

In some embodiments, the engineered DNA polymerase comprises one or more substitutions or substitution sets selected from 12, 13, 14, 15, 17, 19/214, 45, 84, 87, 95, 122, 159, 170/194/476/557/659, 170/423/476/658/685, 170/423/557/658/743, 170/476/557/658/659, 170/476/557/743, 178, 194/658/660, 198, 209, 214, 222, 228, 249, 252, 252/258/331, 252/269, 252/269/412/674, 252/269/674, 252/331, 252/331/352/412/506/674, 252/331/467/660/674, 252/331/660, 252/331/699, 252/352/412, 252/412, 252/412/467/660/699/704, 252/413, 252/467/506/674/704, 252/704, 264, 268, 268/621, 269/331/699, 269/412/467/704, 269/467/506, 269/674, 269/695, 269/699/704, 275, 276, 301, 323, 331/358, 331/412/660/699, 331/674, 331/704, 345, 352/674, 412/467/660/674/699, 412/660, 412/674, 430, 430/837, 433, 467, 470, 476/557/658/659, 476/658/660/743, 477, 478, 485, 492, 511, 514, 517, 520, 533, 534, 534/670, 549, 557/658/659, 565, 596, 598, 642, 658/659, 674, 689, 695, 696, 699/704, 707, 725, and 783, or combinations thereof, wherein the amino acid positions are relative to the reference sequence of SEQ ID NO: 178.

In some embodiments, the engineered DNA polymerase comprises one or more substitutions or substitution sets selected from 12E, 12V, 13G, 14E, 14V, 15V, 17G, 17K, 17S, 19L/214R, 45S, 84N, 84T, 87A, 87R, 87S, 95D, 95G, 122Q, 159Q, 170L/194V/476L/557R/659P, 170L/423Q/476L/658V/685A, 170L/423Q/557R/658V/743T, 170L/476L/557R/658V/659P, 170L/476L/557R/743T, 178G, 178L, 178N, 178S, 194V/658V/660S, 198V, 209S, 209W, 214R, 222I, 228D, 228S, 249R, 252L, 252L/258H/331S, 252L/269R, 252L/269R/412A/674V, 252L/269R/674V, 252L/331S, 252L/331S/352K/412A/506N/674V, 252L/331S/467N/660P/674V, 252L/331S/660P, 252L/331S/699V, 252L/352K/412A, 252L/412A, 252L/412A/467N/660P/699V/704K, 252L/413K, 252L/467N/506N/674V/704K, 252L/704K, 264T, 268A/621N, 268G, 269C/695R, 269R/331S/699V, 269R/412A/467N/704K, 269R/467N/506N, 269R/674V, 269R/699V/704K, 275E, 275G, 275M, 275V, 276A, 276D, 276G, 276M, 301G, 323L, 323Q, 323S, 331S/358N, 331S/412A/660P/699V, 331S/674V, 331S/704K, 345G, 345P, 352K/674V, 412A/467N/660P/674V/699V, 412A/660P, 412A/674V, 430A/837S, 430F, 433K, 433R, 433S, 433V, 467N, 470M, 470R, 476L/557R/658V/659P, 476L/658V/660S/743T, 477E, 478G, 478L, 478M, 478S, 485A, 485G, 485I, 485T, 485W, 492R, 492Y, 511C, 511L, 514G, 514V, 517A, 517H, 517L, 517R, 517T, 520V, 533L, 533P, 534A, 534A/670V, 549V, 557R/658V/659P, 565R, 596S, 598G, 598K, 598L, 598R, 642E, 642R, 658V/659P, 674V, 689D, 695K, 695S, 695T, 696G, 696K, 696T, 699V/704K, 707A, 725H, and 783H, or combinations thereof, wherein the amino acid positions are relative to the reference sequence of SEQ ID NO: 178.

In some embodiments, the engineered DNA polymerase comprises one or more substitutions or substitution sets selected from M12E, M12V, L13G, P14E, P14V, L15V, E17G, E17K, E17S, K19L/K214R, R45S, E84N, E84T, E87A, E87R, E87S, P95D, P95G, P122Q, P159Q, R170L/A194V/M476L/P557R/S659P, R170L/A423Q/M476L/L658V/H685A, R170L/A423Q/P557R/L658V/N743T, R170L/M476L/P557R/L658V/S659P, R170L/M476L/P557R/N743T, E178G, E178L, E178N, E178S, A194V/L658V/A660S, S198V, E209S, E209W, K214R, L222I, N228D, N228S, K249R, R252L, R252L/R258H/L331S, R252L/G269R, R252L/G269R/G412A/1674V, R252L/G269R/1674V, R252L/L331S, R252L/L331S/R352K/G412A/E506N/1674V, R252L/L331S/A467N/A660P/1674V, R252L/L331S/A660P, R252L/L331S/A699V, R252L/R352K/G412A, R252L/G412A, R252L/G412A/A467N/A660P/A699V/R704K, R252L/E413K, R252L/A467N/E506N/1674V/R704K, R252L/R704K, E264T, R268A/S621N, R268G, G269C/Y695R, G269R/L331S/A699V, G269R/G412A/A467N/R704K, G269R/A467N/E506N, G269R/1674V, G269R/A699V/R704K, W275E, W275G, W275M, W275V, E276A, E276D, E276G, E276M, L301G, P323L, P323Q, P323S, L331S/R358N, L331S/G412A/A660P/A699V, L331S/1674V, L331S/R704K, E345G, E345P, R352K/1674V, G412A/A467N/A660P/1674V/A699V, G412A/A660P, G412A/1674V, Q430A/L837S, Q430F, E433K, E433R, E433S, E433V, A467N, L470M, L470R, M476L/P557R/L658V/S659P, M476L/L658V/A660S/N743T, R477E, R478G, R478L, R478M, R478S, R485A, R485G, R485I, R485T, R485W, N492R, N492Y, P511C, P511L, K514G, K514V, K517A, K517H, K517L, K517R, K517T, K520V, E533L, E533P, V534A, V534A/A670V, K549V, P557R/L658V/S659P, K565R, R596S, P598G, P598K, P598L, P598R, Q642E, Q642R, L658V/S659P, 1674V, Q689D, Y695K, Y695S, Y695T, E696G, E696K, E696T, A699V/R704K, Q707A, T725H, and E783H, or combinations thereof, wherein the amino acid positions are relative to the reference sequence of SEQ ID NO: 178.

In some embodiments, the engineered DNA polymerase comprises one or more substitutions or substitution sets selected from 12, 13, 14, 15, 17, 84, 87, 122, 159, 170/423/476/658/685, 170/476/557/743, 178, 198, 205, 209, 222, 228, 249, 252, 252/269, 252/269/412/674, 252/269/674, 252/331, 252/331/352/412/506/674, 252/331/467/660/674, 252/331/699, 252/412, 252/412/467/660/699/704, 252/413, 252/467/506/674/704, 252/704, 264, 269/331/699, 269/412/467/704, 269/467/506, 269/674, 275, 276, 301, 322, 323, 331/358, 331/412/660/699, 331/674, 331/704, 345, 352/674, 355, 412/674, 430, 430/837, 433, 467, 470, 476/658/660/743, 477, 478, 485, 492, 511, 514, 517, 520, 533, 534, 534/670, 549, 557/658/659, 565, 596, 598, 642, 674, 689, 695, 696, 699/704, 703, 725, and 821, or combinations thereof, wherein the amino acid positions are relative to the reference sequence of SEQ ID NO: 178.

In some embodiments, the engineered DNA polymerase comprises one or more substitutions or substitution sets selected from 12S, 12V, 13G, 14V, 15V, 17G, 17K, 17S, 84N, 84T, 87A, 87Q, 87R, 87S, 122Q, 159Q, 170L/423Q/476L/658V/685A, 170L/476L/557R/743T, 178G, 178L, 178N, 178S, 198V, 205R, 209S, 2221, 228D, 228S, 249G, 249R, 252L, 252L/269R, 252L/269R/412A/674V, 252L/269R/674V, 252L/331S, 252L/331S/352K/412A/506N/674V, 252L/331S/467N/660P/674V, 252L/331S/699V, 252L/412A, 252L/412A/467N/660P/699V/704K, 252L/413K, 252L/467N/506N/674V/704K, 252L/704K, 264S, 264T, 269R/331S/699V, 269R/412A/467N/704K, 269R/467N/506N, 269R/674V, 275E, 275G, 275V, 276A, 276D, 276G, 276M, 301G, 322L, 323L, 331S/358N, 331S/412A/660P/699V, 331S/674V, 331S/704K, 345G, 352K/674V, 355F, 412A/674V, 430A/837S, 430F, 433R, 433S, 433V, 467N, 470E, 470M, 470R, 476L/658V/660S/743T, 477E, 478G, 478M, 485A, 485E, 485G, 485I, 485W, 492R, 492Y, 511C, 511E, 511L, 514G, 514V, 517A, 517H, 517T, 520V, 533L, 533P, 534A, 534A/670V, 549V, 557R/658V/659P, 565R, 565Y, 596S, 598G, 598L, 642R, 674V, 689D, 695K, 695S, 695T, 696G, 696K, 696T, 696W, 699V/704K, 703K, 725L, and 821R, or combinations thereof, wherein the amino acid positions are relative to the reference sequence of SEQ ID NO: 178.

In some embodiments, the engineered DNA polymerase comprises one or more substitutions or substitution sets selected from M12S, M12V, L13G, P14V, L15V, E17G, E17K, E17S, E84N, E84T, E87A, E87Q, E87R, E87S, P122Q, P159Q, R170L/A423Q/M476L/L658V/H685A, R170L/M476L/P557R/N743T, E178G, E178L, E178N, E178S, S198V, K205R, E209S, L222I, N228D, N228S, K249G, K249R, R252L, R252L/G269R, R252L/G269R/G412A/1674V, R252L/G269R/I674V, R252L/L331S, R252L/L331S/R352K/G412A/E506N/1674V, R252L/L331S/A467N/A660P/1674V, R252L/L331S/A699V, R252L/G412A, R252L/G412A/A467N/A660P/A699V/R704K, R252L/E413K, R252L/A467N/E506N/1674V/R704K, R252L/R704K, E264S, E264T, G269R/L331S/A699V, G269R/G412A/A467N/R704K, G269R/A467N/E506N, G269R/1674V, W275E, W275G, W275V, E276A, E276D, E276G, E276M, L301G, R322L, P323L, L331S/R358N, L331S/G412A/A660P/A699V, L331S/1674V, L331S/R704K, E345G, R352K/1674V, K355F, G412A/1674V, Q430A/L837S, Q430F, E433R, E433S, E433V, A467N, L470E, L470M, L470R, M476L/L658V/A660S/N743T, R477E, R478G, R478M, R485A, R485E, R485G, R485I, R485W, N492R, N492Y, P511C, P511E, P511L, K514G, K514V, K517A, K517H, K517T, K520V, E533L, E533P, V534A, V534A/A670V, K549V, P557R/L658V/S659P, K565R, K565Y, R596S, P598G, P598L, Q642R, 1674V, Q689D, Y695K, Y695S, Y695T, E696G, E696K, E696T, E696W, A699V/R704K, Q703K, T725L, and P821R, or combinations thereof, wherein the amino acid positions are relative to the reference sequence of SEQ ID NO: 178.

In some embodiments, the engineered DNA polymerase comprises one or more substitutions or substitution sets selected from 12, 15, 17, 252, 252/331, 252/331/352/412/506/674, 252/413, 252/704, 269/412/467/704, 269/467/506, 269/674, 301, 331/704, 345, 352/674, 355, 430/837, 492, 511, 514, 520, 598, 642, 674, 695, 696, 699/704, and 703, or combinations thereof, wherein the amino acid positions are relative to the reference sequence of SEQ ID NO: 178.

In some embodiments, the engineered DNA polymerase comprises one or more substitutions or substitution sets selected from 12V, 15V, 17G, 17K, 252L, 252L/331S, 252L/331S/352K/412A/506N/674V, 252L/413K, 252L/704K, 269R/412A/467N/704K, 269R/467N/506N, 269R/674V, 301G, 331S/704K, 345G, 352K/674V, 355F, 430A/837S, 492R, 492Y, 511C, 511L, 514G, 514V, 520V, 598G, 642R, 674V, 695S, 695T, 696T, 699V/704K, and 703K, or combinations thereof, wherein the amino acid positions are relative to the reference sequence of SEQ ID NO: 178.

In some embodiments, the engineered DNA polymerase comprises one or more substitutions or substitution sets selected from M12V, L15V, E17G, E17K, R252L, R252L/L331S, R252L/L331S/R352K/G412A/E506N/1674V, R252L/E413K, R252L/R704K, G269R/G412A/A467N/R704K, G269R/A467N/E506N, G269R/1674V, L301G, L331S/R704K, E345G, R352K/1674V, K355F, Q430A/L837S, N492R, N492Y, P511C, P511L, K514G, K514V, K520V, P598G, Q642R, 1674V, Y695S, Y695T, E696T, A699V/R704K, and Q703K, or combinations thereof, wherein the amino acid positions are relative to the reference sequence of SEQ ID NO: 178.

The present invention also provides engineered DNA polymerases or a functional fragments thereof, comprising a polypeptide sequence having at least 75% or more sequence identity to a reference sequence comprising residues 12 to 841 of SEQ ID NO: 994, or to a reference sequence of SEQ ID NO: 994, wherein the polypeptide sequence comprises one or more substitutions relative to the reference sequence comprising residues 12 to 841 of SEQ ID NO: 994, or to the reference sequence of SEQ ID NO: 994.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 994 or to the reference sequence of SEQ ID NO: 994.

In some embodiments, the engineered DNA polymerase comprises one or more substitutions or substitution sets selected from 2, 5, 7, 8, 12/15/269/355/412/413/492/704, 12/252/413/492, 32, 33, 35/304, 38, 40, 65, 80, 81, 140, 151, 169, 194, 194/252, 194/252/358, 194/252/358/412/467, 194/252/358/412/467/658/696/743, 194/252/412/696, 194/252/467/696/743, 194/252/743, 194/358, 194/358/412, 194/358/658, 194/358/704, 194/412, 194/412/467, 194/412/467/743, 194/467/511/658, 194/467/658, 194/467/704, 194/467/743, 194/743, 218, 226, 242, 252, 252/412/658, 252/467/696, 258/347, 279, 284, 289, 298, 300, 304, 311, 329, 331, 340, 358, 375, 375/376, 406, 420, 434, 440, 481, 489, 490, 518, 529, 543, 560, 592, 635, 639, 645, 658, 658/704, 666, 685, 694, 704, 713, 717, 718, 721, 740, 746, 782, 804, 807, 814, and 829, or combinations thereof, wherein the amino acid positions are relative to the reference sequence of SEQ ID NO: 994.

In some embodiments, the engineered DNA polymerase comprises one or more substitutions or substitution sets selected from 2G, 2R, 5A, 5G, 5V, 5W, 7Q, 8R, 12V/15V/269R/355F/412A/413K/492R/704K, 12V/252L/413K/492R, 32L, 33K, 35L/304G, 38E, 38G, 38R, 38S, 38V, 40N, 40R, 40S, 65S, 80G, 81A, 140M, 151K, 169S, 194V, 194V/252L, 194V/252L/358N, 194V/252L/358N/412A/467N, 194V/252L/358N/412A/467N/658V/696T/743T, 194V/252L/412A/696T, 194V/252L/467N/696T/743T, 194V/252L/743T, 194V/358N, 194V/358N/412A, 194V/358N/658V, 194V/358N/704K, 194V/412A, 194V/412A/467N, 194V/412A/467N/743T, 194V/467N/511C/658V, 194V/467N/658V, 194V/467N/704K, 194V/467N/743T, 194V/743T, 218T, 218Y, 226E, 242G, 242N, 252L, 252L/412A/658V, 252L/467N/696T, 258L/347H, 279R, 284A, 289D, 289G, 298A, 298S, 300A, 300F, 300L, 300N, 300R, 300T, 300W, 304C, 304G, 304T, 304V, 311C, 329L, 329S, 331D, 331Q, 331Y, 340T, 358N, 375A/376I, 375I, 406M, 420S, 420T, 434K, 440N, 440T, 481D, 481G, 489R, 490R, 490V, 518L, 518R, 529R, 529T, 529V, 543A, 560G, 560K, 592A, 592S, 635I, 639Q, 645L, 645M, 645R, 658A, 658C, 658E, 658R, 658V/704K, 666D, 666G, 666K, 666M, 666N, 666R, 666S, 666T, 666V, 685T, 694S, 694T, 704Q, 704V, 713V, 717T, 718R, 718S, 721V, 721W, 740G, 746G, 746Q, 746R, 782S, 804D, 804Q, 807H, 814A, 814I, 814R, 829L, and 829R, or combinations thereof, wherein the amino acid positions are relative to the reference sequence of SEQ ID NO: 994.

In some embodiments, the engineered DNA polymerase comprises one or more substitutions or substitution sets selected from H2G, H2R, H5A, H5G, H5V, H5W, H7Q, G8R, M12V/L15V/G269R/K355F/G412A/E413K/Y492R/R704K, M12V/R252L/E413K/Y492R, Y32L, R33K, F35L/E304G, L38E, L38G, L38R, L38S, L38V, G40N, G40R, G40S, E65S, S80G, F81A, E140M, R151K, H169S, A194V, A194V/R252L, A194V/R252L/R358N, A194V/R252L/R358N/G412A/A467N, A194V/R252L/R358N/G412A/A467N/L658V/E696T/N743T, A194V/R252L/G412A/E696T, A194V/R252L/A467N/E696T/N743T, A194V/R252L/N743T, A194V/R358N, A194V/R358N/G412A, A194V/R358N/L658V, A194V/R358N/R704K, A194V/G412A, A194V/G412A/A467N, A194V/G412A/A467N/N743T, A194V/A467N/P511C/L658V, A194V/A467N/L658V, A194V/A467N/R704K, A194V/A467N/N743T, A194V/N743T, E218T, E218Y, L226E, L242G, L242N, R252L, R252L/G412A/L658V, R252L/A467N/E696T, R258L/P347H, K279R, R284A, S289D, S289G, E298A, E298S, P300A, P300F, P300L, P300N, P300R, P300T, P300W, E304C, E304G, E304T, E304V, P311C, E329L, E329S, L331D, L331Q, L331Y, R340T, R358N, D375A/L376I, D375I, E406M, R420S, R420T, R434K, E440N, E440T, E481D, E481G, H489R, S490R, S490V, T518L, T518R, E529R, E529T, E529V, Q543A, A560G, A560K, N592A, N592S, E635I, R639Q, K645L, K645M, K645R, L658A, L658C, L658E, L658R, L658V/R704K, L666D, L666G, L666K, L666M, L666N, L666R, L666S, L666T, L666V, H685T, P694S, P694T, R704Q, R704V, K713V, E717T, K718R, K718S, E721V, E721W, P740G, V746G, V746Q, V746R, R782S, R804D, R804Q, E807H, E814A, E814I, E814R, E829L, and E829, or combinations thereof, wherein the amino acid positions are relative to the reference sequence of SEQ ID NO: 994.

In some embodiments, the engineered DNA polymerase comprises one or more substitutions or substitution sets selected from 2, 5, 8, 12/15/269/355/412/413/492/704, 12/252/413/492, 38, 40, 65, 80, 140, 151, 218, 226, 230, 242, 258/347, 279, 284, 289, 298, 300, 304, 309, 311, 329, 331, 340, 375, 375/376, 406, 420, 434, 441, 489, 490, 518, 529, 543, 560, 592, 635, 639, 645, 658, 666, 685, 704, 713, 717, 718, 721, 740, 743, 746, 782, 804, 807, 814, and 829, or combinations thereof, wherein the amino acid positions are relative to the reference sequence of SEQ ID NO: 994.

In some embodiments, the engineered DNA polymerase comprises one or more substitutions or substitution sets selected from 2G, 2V, 5A, 5G, 5L, 5V, 5W, 8R, 12V/15V/269R/355F/412A/413K/492R/704K, 12V/252L/413K/492R, 38G, 38R, 38S, 38V, 40R, 40S, 65G, 65H, 65S, 80G, 80M, 140M, 151K, 218Y, 226E, 230G, 242A, 242G, 242R, 258L/347H, 279R, 284A, 284G, 289D, 298A, 298S, 300F, 300N, 300R, 304C, 304G, 304R, 304V, 309G, 311C, 311G, 329L, 331D, 331G, 331Y, 340T, 375A/376I, 375E, 375L, 406G, 420S, 420T, 434K, 441R, 489R, 490R, 490V, 518M, 518R, 529V, 543A, 543L, 543R, 560G, 592A, 592S, 635I, 639Q, 645R, 658Q, 658R, 666G, 666Q, 666R, 666V, 685F, 704V, 713V, 717T, 718R, 721D, 721M, 721R, 721W, 740L, 740S, 743S, 746Q, 782A, 804G, 807G, 807H, 814I, 814M, 814R, 829L, and 829R, or combinations thereof, wherein the amino acid positions are relative to the reference sequence of SEQ ID NO: 994.

In some embodiments, the engineered DNA polymerase comprises one or more substitutions or substitution sets selected from H2G, H2V, H5A, H5G, H5L, H5V, H5W, G8R, M12V/L15V/G269R/K355F/G412A/E413K/Y492R/R704K, M12V/R252L/E413K/Y492R, L38G, L38R, L38S, L38V, G40R, G40S, E65G, E65H, E65S, S80G, S80M, E140M, R151K, E218Y, L226E, D230G, L242A, L242G, L242R, R258L/P347H, K279R, R284A, R284G, S289D, E298A, E298S, P300F, P300N, P300R, E304C, E304G, E304R, E304V, P309G, P311C, P311G, E329L, L331D, L331G, L331Y, R340T, D375A/L376I, D375E, D375L, E406G, R420S, R420T, R434K, E441R, H489R, S490R, S490V, T518M, T518R, E529V, Q543A, Q543L, Q543R, A560G, N592A, N592S, E635I, R639Q, K645R, L658Q, L658R, L666G, L666Q, L666R, L666V, H685F, R704V, K713V, E717T, K718R, E721D, E721M, E721R, E721W, P740L, P740S, N743S, V746Q, R782A, R804G, E807G, E807H, E814I, E814M, E814R, E829L, and E829R, or combinations thereof, wherein the amino acid positions are relative to the reference sequence of SEQ ID NO: 994.

In some embodiments, the engineered DNA polymerase comprises one or more substitutions or substitution sets selected from 2, 5, 8, 12/15/269/355/412/413/492/704, 12/252/413/492, 32, 38, 40, 65, 98, 140, 151, 169, 202, 218, 226, 227, 230, 242, 258/347, 279, 284, 298, 300, 304, 309, 311, 329, 331, 340, 375, 375/376, 406, 420, 434, 440, 441, 481, 489, 490, 518, 529, 543, 560, 592, 635, 639, 645, 658, 666, 685, 692, 694, 704, 713, 717, 718, 721, 740, 746, 782, 804, 807, 814, and 829, or combinations thereof, wherein the amino acid positions are relative to the reference sequence of SEQ ID NO: 994.

In some embodiments, the engineered DNA polymerase comprises one or more substitutions or substitution sets selected from 2G, 2P, 2V, 5L, 5V, 8R, 12V/15V/269R/355F/412A/413K/492R/704K, 12V/252L/413K/492R, 32L, 38V, 40N, 40S, 65G, 65H, 65S, 98G, 98N, 140M, 151K, 169S, 202Q, 218G, 218Y, 226E, 227Y, 230G, 242A, 242G, 242R, 258L/347H, 279R, 284G, 298A, 298S, 300A, 300F, 300T, 304C, 304G, 304V, 309G, 311G, 329L, 329S, 331G, 331Q, 331V, 331Y, 340S, 340T, 375A, 375A/376I, 375E, 375L, 375M, 375R, 406M, 406Q, 420S, 420T, 434K, 440S, 440T, 441G, 481D, 481G, 489R, 490R, 490V, 518L, 518M, 529L, 529T, 529V, 543A, 543L, 543R, 560G, 560K, 592A, 592S, 635G, 635I, 635T, 639Q, 645F, 645L, 645M, 645N, 645R, 658E, 658Q, 658R, 658S, 666D, 666E, 666G, 666K, 666N, 666Q, 666R, 666S, 666V, 685F, 692S, 694S, 704Q, 704V, 713S, 713V, 717T, 717Y, 718S, 721D, 721L, 721M, 721R, 721V, 721W, 740L, 740S, 746G, 746Q, 746R, 782A, 782S, 804A, 804D, 807G, 814A, 814M, 814R, 829L, and 829R, or combinations thereof, wherein the amino acid positions are relative to the reference sequence of SEQ ID NO: 994.

In some embodiments, the engineered DNA polymerase comprises one or more substitutions or substitution sets selected from H2G, H2P, H2V, H5L, H5V, G8R, M12V/L15V/G269R/K355F/G412A/E413K/Y492R/R704K, M12V/R252L/E413K/Y492R, Y32L, L38V, G40N, G40S, E65G, E65H, E65S, E98G, E98N, E140M, R151K, H169S, P202Q, E218G, E218Y, L226E, K227Y, D230G, L242A, L242G, L242R, R258L/P347H, K279R, R284G, E298A, E298S, P300A, P300F, P300T, E304C, E304G, E304V, P309G, P311G, E329L, E329S, L331G, L331Q, L331V, L331Y, R340S, R340T, D375A, D375A/L376I, D375E, D375L, D375M, D375R, E406M, E406Q, R420S, R420T, R434K, E440S, E440T, E441G, E481D, E481G, H489R, S490R, S490V, T518L, T518M, E529L, E529T, E529V, Q543A, Q543L, Q543R, A560G, A560K, N592A, N592S, E635G, E635I, E635T, R639Q, K645F, K645L, K645M, K645N, K645R, L658E, L658Q, L658R, L658S, L666D, L666E, L666G, L666K, L666N, L666Q, L666R, L666S, L666V, H685F, G692S, P694S, R704Q, R704V, K713S, K713V, E717T, E717Y, K718S, E721D, E721L, E721M, E721R, E721V, E721W, P740L, P740S, V746G, V746Q, V746R, R782A, R782S, R804A, R804D, E807G, E814A, E814M, E814R, E829L, and E829R, or combinations thereof, wherein the amino acid positions are relative to the reference sequence of SEQ ID NO: 994.

In some embodiments, the engineered DNA polymerase comprises one or more substitutions or substitution sets selected from 12/15/269/355/412/413/492/704, 12/252/413/492, 194, 194/252, 194/252/358/412/467, 194/252/358/412/467/658/696/743, 194/252/412/696, 194/252/467/658, 194/252/467/696/743, 194/252/467/704, 194/252/696/704, 194/252/704/743, 194/252/743, 194/358, 194/358/412, 194/358/467/658/696/704, 194/358/658, 194/412, 194/412/467, 194/412/658, 194/467, 194/467/658, 194/467/658/743, 194/467/704, 194/467/743, 194/658, 194/743, 242, 252/358/696/704, 252/412/658, 252/467/696, 258/347, 284, 304, 331, 434, 489, 543, 560, 592, 658, 666, 685, 717, 721, and 814, or combinations thereof, wherein the amino acid positions are relative to the reference sequence of SEQ ID NO: 994.

In some embodiments, the engineered DNA polymerase comprises one or more substitutions or substitution sets selected from 12V/15V/269R/355F/412A/413K/492R/704K, 12V/252L/413K/492R, 194V, 194V/252L, 194V/252L/358N/412A/467N, 194V/252L/358N/412A/467N/658V/696T/743T, 194V/252L/412A/696T, 194V/252L/467N/658V, 194V/252L/467N/696T/743T, 194V/252L/467N/704K, 194V/252L/696T/704K, 194V/252L/704K/743T, 194V/252L/743T, 194V/358N, 194V/358N/412A, 194V/358N/467N/658V/696T/704K, 194V/358N/658V, 194V/412A, 194V/412A/467N, 194V/412A/658V, 194V/467N, 194V/467N/658V, 194V/467N/658V/743T, 194V/467N/704K, 194V/467N/743T, 194V/658V, 194V/743T, 242G, 252L/358N/696T/704K, 252L/412A/658V, 252L/467N/696T, 258L/347H, 284A, 304G, 304V, 331G, 434K, 489R, 543A, 560G, 592A, 658E, 666R, 685F, 717T, 721M, 721R, and 814M, or combinations thereof, wherein the amino acid positions are relative to the reference sequence of SEQ ID NO: 994.

In some embodiments, the engineered DNA polymerase comprises one or more substitutions or substitution sets selected from M12V/L15V/G269R/K355F/G412A/E413K/Y492R/R704K, M12V/R252L/E413K/Y492R, A194V, A194V/R252L, A194V/R252L/R358N/G412A/A467N, A194V/R252L/R358N/G412A/A467N/L658V/E696T/N743T, A194V/R252L/G412A/E696T, A194V/R252L/A467N/L658V, A194V/R252L/A467N/E696T/N743T, A194V/R252L/A467N/R704K, A194V/R252L/E696T/R704K, A194V/R252L/R704K/N743T, A194V/R252L/N743T, A194V/R358N, A194V/R358N/G412A, A194V/R358N/A467N/L658V/E696T/R704K, A194V/R358N/L658V, A194V/G412A, A194V/G412A/A467N, A194V/G412A/L658V, A194V/A467N, A194V/A467N/L658V, A194V/A467N/L658V/N743T, A194V/A467N/R704K, A194V/A467N/N743T, A194V/L658V, A194V/N743T, L242G, R252L/R358N/E696T/R704K, R252L/G412A/L658V, R252L/A467N/E696T, R258L/P347H, R284A, E304G, E304V, L331G, R434K, H489R, Q543A, A560G, N592A, L658E, L666R, H685F, E717T, E721M, E721R, and 814M, or combinations thereof, wherein the amino acid positions are relative to the reference sequence of SEQ ID NO: 994.

The present invention further provides engineered DNA polymerases or functional fragments thereof, comprising a polypeptide sequence having at least 75% or more sequence identity to a reference sequence comprising residues 12 to 841 of SEQ ID NO: 1858, or to a reference sequence of SEQ ID NO: 1858, wherein the polypeptide sequence comprises one or more substitutions relative to the reference sequence comprising residues 12 to 841 of SEQ ID NO: 1858, or to the reference sequence of SEQ ID NO: 1858.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 1858 or to the reference sequence of SEQ ID NO: 1858.

In some embodiments, the engineered DNA polymerase comprises one or more substitutions or substitution sets selected from 194/252/269/304/467, 194/252/304, 194/252/304/358/467/658, 194/252/304/467, 194/252/358/489, 194/252/467, 194/269, 194/269/304, 194/269/304/658/717, 194/269/717, 194/304/358/467/489, 194/467/721, 194/536/658, 194/721, 252, 252/269/304/743, 252/304/489/696/717, 252/304/658, 252/358/467/489/685, 252/685, 269, 269/304, 269/304/467, 269/304/658, 269/658, 269/721, 304, 304/358/467/592, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

In some embodiments, the engineered DNA polymerase comprises one or more substitutions or substitution sets selected from 194V/252L/269G/304G/467N, 194V/252L/304G, 194V/252L/304G/358N/467N/658E, 194V/252L/304G/467N, 194V/252L/358N/489R, 194V/252L/467N, 194V/269G, 194V/269G/304G, 194V/269G/304V/658V/717T, 194V/269G/717T, 194V/304V/358N/467N/489R, 194V/467N/721R, 194V/536G/658E, 194V/721R, 252L, 252L/269G/304V/743T, 252L/304G/489R/696T/717T, 252L/304G/658V, 252L/358N/467N/489R/685F, 252L/685F, 269G, 269G/304G, 269G/304G/658E, 269G/304V/467N, 269G/658V, 269G/721R, 304G, 304G/358N/467N/592A, 304G/467N/489R/717T, 304G/489R/658V/721R, 304G/685F, 304V, 304V/489R/658E, 358N/658V, 489R, 489R/696T/743T, 685F, 696T, 717T, and 721R, or combinations thereof, wherein the amino acid positions are relative to the reference sequence of SEQ ID NO: 1858.

In some embodiments, the engineered DNA polymerase comprises one or more substitutions or substitution sets selected from A194V/R252L/R269G/E304G/A467N, A194V/R252L/E304G, A194V/R252L/E304G/R358N/A467N/L658E, A194V/R252L/E304G/A467N, A194V/R252L/R358N/H489R, A194V/R252L/A467N, A194V/R269G, A194V/R269G/E304G, A194V/R269G/E304V/L658V/E717T, A194V/R269G/E717T, A194V/E304V/R358N/A467N/H489R, A194V/A467N/E721R, A194V/P536G/L658E, A194V/E721R, R252L, R252L/R269G/E304V/N743T, R252L/E304G/H489R/E696T/E717T, R252L/E304G/L658V, R252L/R358N/A467N/H489R/H685F, R252L/H685F, R269G, R269G/E304G, R269G/E304G/L658E, R269G/E304V/A467N, R269G/L658V, R269G/E721R, E304G, E304G/R358N/A467N/N592A, E304G/A467N/H489R/E717T, E304G/H489R/L658V/E721R, E304G/H685F, E304V, E304V/H489R/L658E, R358N/L658V, H489R, H489R/E696T/N743T, H685F, E696T, E717T, and E721R, or combinations thereof, wherein the amino acid positions are relative to the reference sequence of SEQ ID NO: 1858.

In some embodiments, the engineered DNA polymerase comprises one or more substitutions or substitution sets selected from 252, 252/304, 252/304/489/696/717, 252/304/658, 252/358/467/489/685, 252/685, 269, 269/304/467, 269/304/489/685, 269/467/489/658/685, 269/658, 269/721, 304, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

In some embodiments, the engineered DNA polymerase comprises one or more substitutions or substitution sets selected from 252L, 252L/304G/489R/696T/717T, 252L/304G/658V, 252L/304V, 252L/358N/467N/489R/685F, 252L/685F, 269G, 269G/304G/489R/685F, 269G/304V/467N, 269G/467N/489R/658V/685F, 269G/658V, 269G/721R, 304G/467N/489R/717T, 304G/489R/658V/721R, 304G/685F, 304V, 304V/489R/658E, 358N/658V, 489R, 489R/696T/743T, 685F, 696T, 717T, and 721R, or combinations thereof, wherein the amino acid positions are relative to the reference sequence of SEQ ID NO: 1858.

In some embodiments, the engineered DNA polymerase comprises one or more substitutions or substitution sets selected from R252L, R252L/E304G/H489R/E696T/E717T, R252L/E304G/L658V, R252L/E304V, R252L/R358N/A467N/H489R/H685F, R252L/H685F, R269G, R269G/E304G/H489R/H685F, R269G/E304V/A467N, R269G/A467N/H489R/L658V/H685F, R269G/L658V, R269G/E721R, E304G/A467N/H489R/E717T, E304G/H489R/L658V/E721R, E304G/H685F, E304V, E304V/H489R/L658E, R358N/L658V, H489R, H489R/E696T/N743T, H685F, E696T, E717T, and E721R, or combinations thereof, wherein the amino acid positions are relative to the reference sequence of SEQ ID NO: 1858.

In some embodiments, the engineered DNA polymerase comprises one or more substitutions or substitution sets selected from 252/685, 269, 269/658, 304/467/489/717, 304/489/658/721, 304/685, 358/658, 489, 685, 696, 717, and 721, or combinations thereof, wherein the amino acid positions are relative to the reference sequence of SEQ ID NO: 1858.

In some embodiments, the engineered DNA polymerase comprises one or more substitutions or substitution sets selected from 252L/685F, 269G, 269G/658V, 304G/467N/489R/717T, 304G/489R/658V/721R, 304G/685F, 358N/658V, 489R, 685F, 696T, 717T, and 721R, or combinations thereof, wherein the amino acid positions are relative to the reference sequence of SEQ ID NO: 1858.

In some embodiments, the engineered DNA polymerase comprises one or more substitutions or substitution sets selected from R252L/H685F, R269G, R269G/L658V, E304G/A467N/H489R/E717T, E304G/H489R/L658V/E721R, E304G/H685F, R358N/L658V, H489R, H685F, E696T, E717T, and E721R, or combinations thereof, wherein the amino acid positions are relative to the reference sequence of SEQ ID NO: 1858.

The present invention also provides engineered DNA polymerases or functional fragments thereof, comprising a polypeptide sequence having at least 75% or more sequence identity to a reference sequence comprising residues 12 to 841 of SEQ ID NO: 2176, or to a reference sequence of SEQ ID NO: 2176, wherein the polypeptide sequence comprises one or more substitutions relative to the reference sequence comprising residues 12 to 841 of SEQ ID NO: 2176, or to the reference sequence of SEQ ID NO: 2176.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2176 or to the reference sequence of SEQ ID NO: 2176.

In some embodiments, the engineered DNA polymerase comprises one or more substitutions or substitution sets selected from 40, 42, 45, 45/65/243/268, 45/109/340/605/814, 45/221/243/340/423, 45/243/312/331/451, 45/243/312/331/557, 45/243/312/340/423/543/814, 45/243/814, 45/268/312/605/748/749/814, 45/268/489/557/748/749, 45/312/340/605, 45/340/423/814, 45/340/557, 45/423, 45/431, 45/451, 45/475/557/748/749, 45/489/557/749, 45/621, 45/699/748/749, 57, 61, 64/293, 65, 65/233/543/621, 65/489/543, 81, 94, 102, 109, 109/340/489/510/659, 118, 124, 145, 151, 177, 186, 194, 194/269/489, 194/358, 210, 211, 221, 223, 227, 227/443/592/617, 227/443/756, 233, 242, 243, 243/268, 252, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 289, 292, 292/430, 298, 300, 301, 301/538/546/621/814, 302, 302/340/489/494/510/748/814, 302/340/494, 302/494, 302/494/525, 302/494/543/659/814, 304/489, 312, 312/331/605/814, 312/340/494, 316, 323, 329, 331, 331/605, 340, 340/489, 340/489/494/543/659/814, 340/489/699, 350, 352, 352/451/489/543/557/721, 355, 355/430, 358/467, 371, 397, 406, 407, 420, 423, 431, 441, 451, 463, 466, 475, 481, 488, 489, 489/494, 489/510, 489/621/814, 489/658, 492, 494, 501, 510, 510/748, 519, 521, 530, 533, 534, 536, 538, 543, 543/699/814, 553, 557, 557/605/699/721, 560, 597, 605, 621, 639, 644, 659, 660, 666, 670, 685, 686, 692, 694, 696, 699, 704, 717, 721, 722, 726, 732, 738, 743, 748, 748/749, 749, 756, 802, 804, 814, 827, 829, and 834, or combinations thereof, wherein the amino acid positions are relative to the reference sequence of SEQ ID NO: 2176.

In some embodiments, the engineered DNA polymerase comprises one or more substitutions or substitution sets selected from 40C, 40S, 42V, 45G, 45L, 45L/621R, 45M, 45S, 45S/65N/243N/268Q, 45S/109H/340T/605K/814T, 45S/221D/243N/340T/423E, 45S/243N/312Q/331A/451G, 45S/243N/312Q/331A/557R, 45S/243N/312Q/340T/423E/543A/814T, 45S/243N/814T, 45S/268G/312Q/605K/748G/749K/814T, 45S/268Q/489R/557R/748G/749K, 45S/312Q/340T/605K, 45S/340T/423E/814T, 45S/340T/557R, 45S/423E, 45S/431T, 45S/451G, 45S/475H/557R/748G/749K, 45S/489R/557R/749K, 45S/699Q/748G/749K, 45V, 571, 57R, 61G, 64E/293H, 65C, 65K/489R/543A, 65N, 65N/233S/543V/621R, 65S, 81W, 94K, 94L, 94M, 94R, 102D, 102E, 102G, 102T, 109C, 109H, 109H/340T/489R/510R/659G, 109T, 109Y, 118L, 118T, 118V, 124A, 145F, 151S, 177F, 186M, 194G, 194V/269G/489R, 194V/358N, 210G, 210R, 210S, 211L, 211R, 221C, 221D, 221T, 223D, 227E, 227E/443D/592S/617V, 227E/443D/756R, 233E, 233L, 233R, 233S, 242Q, 242V, 243N, 243N/268G, 243S, 252L, 268E, 268G, 268G/423E/814T, 268G/431T/605K/749K, 268K, 268Q, 268Q/312Q/331A/340T/699Q, 268Q/312Q/331A/423E/451G/557R/605K, 268Q/340T/352Q/423E/451G, 268Q/423E, 268Q/423E/431T/543A/557R/605K/814T, 268Q/423E/814T, 268V, 269G, 269G/658E, 2691, 269L, 269M, 269S, 269V, 272L, 272V, 280E, 280G, 280K, 283S, 284E, 284G, 289E, 292E, 292E/430H, 292G, 292M, 292R, 292W, 298G, 298I, 300E, 300G, 300L, 300M, 300S, 300T, 301C, 301E, 301G, 301G/538M/546A/621R/814T, 302L/340T/489R/494S/510R/748G/814T, 302V, 302V/340T/494V, 302V/494V, 302V/494V/525R, 302V/494V/543V/659G/814T, 304G/489R, 312D, 312Q, 312Q/331A/605K/814T, 312Q/340T/494V, 316T, 323C, 329M, 329S, 329T, 329V, 331A, 331K, 331M, 331S, 331S/605K, 331V, 340A, 340K, 340L, 340T, 340T/489R, 340T/489R/494V/543A/659G/814T, 340T/489R/699Q, 340V, 350C, 350G, 350S, 352Q, 352Q/451G/489R/543V/557R/721R, 355C, 355L/430R, 355T, 355V, 358N/467N, 371C, 371S, 397V, 406G, 406L, 406W, 407L, 407V, 420H, 423E, 423R, 431T, 431V, 441L, 441M, 451G, 463L, 463W, 466H, 466K, 466M, 475C, 475H, 481A, 481S, 488A, 488R, 488S, 489R, 489R/494V, 489R/510R, 489R/621R/814T, 489R/658E, 492A, 494H, 494R, 494V, 494Y, 501V, 510D, 510L, 510M, 510R, 510R/748G, 519F, 519L, 519V, 521W, 530D, 530E, 530G, 530T, 530V, 530W, 533P, 533T, 534A, 534G, 534R, 534W, 536D, 536G, 536T, 538M, 543A, 543L, 543V/699S/814T, 553Q, 553V, 557L, 557M, 557R, 557R/605K/699Q/721R, 557S, 560M, 560S, 560V, 597G, 597N, 597S, 597V, 605K, 621D, 621E, 621G, 621K, 621Q, 621R, 621T, 639K, 644R, 659A, 659C, 659T, 660G, 660V, 666S, 666W, 670M, 685G, 685V, 686G, 692R, 694T, 696Q, 699G, 699Q, 699S, 704R, 717D, 721A, 721R, 722V, 722W, 726M, 732M, 738P, 743G, 748G, 748G/749K, 749G, 749K, 749S, 749T, 756I, 756L, 802C, 802R, 804A, 804G, 814G, 814K, 814M, 814Q, 814R, 827R, 829R, and 834L, or combinations thereof, wherein the amino acid positions are relative to the reference sequence of SEQ ID NO: 2176.

In some embodiments, the engineered DNA polymerase comprises one or more substitutions or substitution sets selected from G40C, G40S, T42V, R45G, R45L, R45L/S621R, R45M, R45S, R45S/E65N/A243N/R268Q, R45S/E109H/R340T/R605K/E814T, R45S/S221D/A243N/R340T/A423E, R45S/A243N/E312Q/L331A/A451G, R45S/A243N/E312Q/L331A/P557R, R45S/A243N/E312Q/R340T/A423E/Q543A/E814T, R45S/A243N/E814T, R45S/R268G/E312Q/R605K/S748G/V749K/E814T, R45S/R268Q/H489R/P557R/S748G/V749K, R45S/E312Q/R340T/R605K, R45S/R340T/A423E/E814T, R45S/R340T/P557R, R45S/A423E, R45S/G431T, R45S/A451G, R45S/E475H/P557R/S748G/V749K, R45S/H489R/P557R/V749K, R45S/A699Q/S748G/V749K, R45V, K57I, K57R, K61G, K64E/E293H, E65C, E65K/H489R/Q543A, E65N, E65N/K233S/Q543V/S621R, E65S, F81W, A94K, A94L, A94M, A94R, R102D, R102E, R102G, R102T, E109C, E109H, E109H/R340T/H489R/P510R/S659G, E109T, E109Y, R118L, R118T, R118V, F124A, Q145F, R151S, W177F, Q186M, A194G, A194V/R269G/H489R, A194V/R358N, K210G, K210R, K210S, T211L, T211R, S221C, S221D, S221T, E223D, K227E, K227E/E443D/N592S/A617V, K227E/E443D/M756R, K233E, K233L, K233R, K233S, L242Q, L242V, A243N, A243N/R268G, A243S, R252L, R268E, R268G, R268G/A423E/E814T, R268G/G431T/R605K/V749K, R268K, R268Q, R268Q/E312Q/L331A/R340T/A699Q, R268Q/E312Q/L331A/A423E/A451G/P557R/R605K, R268Q/R340T/R352Q/A423E/A451G, R268Q/A423E, R268Q/A423E/G431T/Q543A/P557R/R605K/E814T, R268Q/A423E/E814T, R268V, R269G, R269G/L658E, R269I, R269L, R269M, R269S, R269V, E272L, E272V, A280E, A280G, A280K, E283S, R284E, R284G, S289E, H292E, H292E/Q430H, H292G, H292M, H292R, H292W, E298G, E298I, P300E, P300G, P300L, P300M, P300S, P300T, L301C, L301E, L301G, L301G/V538M/E546A/S621R/E814T, P302L/R340T/H489R/N494S/P510R/S748G/E814T, P302V, P302V/R340T/N494V, P302V/N494V, P302V/N494V/A525R, P302V/N494V/Q543V/S659G/E814T, E304G/H489R, E312D, E312Q, E312Q/L331A/R605K/E814T, E312Q/R340T/N494V, L316T, P323C, E329M, E329S, E329T, E329V, L331A, L331K, L331M, L331S, L331S/R605K, L331V, R340A, R340K, R340L, R340T, R340T/H489R, R340T/H489R/N494V/Q543A/S659G/E814T, R340T/H489R/A699Q, R340V, A350C, A350G, A350S, R352Q, R352Q/A451G/H489R/Q543V/P557R/E721R, F355C, F355L/Q430R, F355T, F355V, R358N/A467N, R371C, R371S, E397V, E406G, E406L, E406W, W407L, W407V, R420H, A423E, A423R, G431T, G431V, E441L, E441M, A451G, A463L, A463W, E466H, E466K, E466M, E475C, E475H, E481A, E481S, G488A, G488R, G488S, H489R, H489R/N494V, H489R/P510R, H489R/S621R/E814T, H489R/L658E, R492A, N494H, N494R, N494V, N494Y, R501V, P510D, P510L, P510M, P510R, P510R/S748G, G519F, G519L, G519V, R521W, A530D, A530E, A530G, A530T, A530V, A530W, E533P, E533T, V534A, V534G, V534R, V534W, P536D, P536G, P536T, V538M, Q543A, Q543L, Q543V/A699S/E814T, T553Q, T553V, P557L, P557M, P557R, P557R/R605K/A699Q/E721R, P557S, A560M, A560S, A560V, T597G, T597N, T597S, T597V, R605K, S621D, S621E, S621G, S621K, S621Q, S621R, S621T, R639K, G644R, S659A, S659C, S659T, A660G, A660V, L666S, L666W, A670M, F685G, F685V, R686G, G692R, P694T, E696Q, A699G, A699Q, A699S, K704R, E717D, E721A, E721R, E722V, E722W, R726M, L732M, Y738P, N743G, S748G, S748G/V749K, V749G, V749K, V749S, V749T, M756I, M756L, K802C, K802R, R804A, R804G, E814G, E814K, E814M, E814Q, E814R, E827R, E829R, and E834L, or combinations thereof, wherein the amino acid positions are relative to the reference sequence of SEQ ID NO: 2176.

In some embodiments, the engineered DNA polymerase comprises one or more substitutions or substitution sets selected from 45, 45/65/243/268, 45/109/243/423/543/605/699/748/749/814, 45/109/340/605/814, 45/221/243/340/423, 45/243/312/331/451, 45/243/312/331/557, 45/243/312/340/423/543/814, 45/243/312/605, 45/243/814, 45/268/312/605/748/749/814, 45/268/489/543/557/659/721, 45/268/489/557/748/749, 45/312, 45/312/340/605, 45/340/423/814, 45/340/475/557/814, 45/340/557, 45/423, 45/431, 45/451, 45/475/557/748/749, 45/489/557/749, 45/621, 45/699/748/749, 64/293, 65, 65/489/543, 81, 94, 102, 109, 109/340/489/510/659, 109/423, 118, 120, 151, 162, 194, 211, 221, 233, 243, 243/268, 245, 252, 258, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 292, 298, 300, 301, 301/538/546/621/814, 302/340/489/494/510/748/814, 302/340/489/494/525, 302/340/494, 302/494, 302/494/525, 304/489, 312, 312/331/605/814, 312/423/431/451/489, 312/494/525/543/659/748/814, 323, 329, 331, 331/605, 340, 340/489, 340/489/494/543/659/814, 340/489/699, 350, 352, 352/451/489/543/557/721, 355, 355/430, 358/467, 406, 407, 423, 423/463/605, 431, 451, 463, 466, 467, 475, 476, 481, 489, 489/494, 489/494/659, 489/510, 489/658, 494, 501, 510, 510/748, 519, 530, 533, 534, 538, 543, 543/699/814, 557, 557/605/699/721, 560, 597, 605, 621, 659, 666, 694, 696, 699, 721, 722, 748, 748/749, 749, 756, 802, and 814, or combinations thereof, wherein the amino acid positions are relative to the reference sequence of SEQ ID NO: 2176.

In some embodiments, the engineered DNA polymerase comprises one or more substitutions or substitution sets selected from 45G, 45L, 45L/621R, 45S, 45S/65N/243N/268Q, 45S/109H/243N/423E/543V/605K/699Q/748G/749K/814T, 45S/109H/340T/605K/814T, 45S/221D/243N/340T/423E, 45S/243N/312Q/331A/451G, 45S/243N/312Q/331A/557R, 45S/243N/312Q/340T/423E/543A/814T, 45S/243N/312Q/605K, 45S/243N/814T, 45S/268G/312Q/605K/748G/749K/814T, 45S/268Q/489R/543V/557R/659T/721R, 45S/268Q/489R/557R/748G/749K, 45S/312Q, 45S/312Q/340T/605K, 45S/340T/423E/814T, 45S/340T/475H/557R/814T, 45S/340T/557R, 45S/423E, 45S/431T, 45S/451G, 45S/475H/557R/748G/749K, 45S/489R/557R/749K, 45S/699Q/748G/749K, 45V, 64H/293D, 65C, 65K/489R/543A, 65N, 65S, 81W, 94K, 102P, 109H, 109H/340T/489R/510R/659G, 109H/423E, 118L, 118V, 120D, 151G, 162T, 194G, 211R, 221D, 233R, 243N, 243N/268G, 243S, 245T, 252L, 258L, 268E, 268G, 268G/423E/814T, 268G/431T/605K/749K, 268Q, 268Q/312Q/331A/340T/699Q, 268Q/312Q/331A/423E/451G/557R/605K, 268Q/340T/352Q/423E/451G, 268Q/423E, 268Q/423E/431T/543A/557R/605K/814T, 268Q/423E/814T, 269G, 269G/658E, 2691, 272V, 280G, 283A, 283L, 283S, 284G, 292R, 298D, 298I, 300E, 300G, 300L, 300M, 300S, 300T, 301C, 301E, 301G, 301G/538M/546A/621R/814T, 302L/340T/489R/494S/510R/748G/814T, 302L/340T/489R/494V/525R, 302V/340T/494V, 302V/494V, 302V/494V/525R, 304G/489R, 312Q, 312Q/331A/605K/814T, 312Q/423E/431T/451G/489R, 312Q/494V/525R/543V/659G/748G/814T, 323W, 329M, 329S, 331A, 331S/605K, 331V, 340L, 340T, 340T/489R, 340T/489R/494V/543A/659G/814T, 340T/489R/699Q, 350C, 350G, 350Y, 352Q, 352Q/451G/489R/543V/557R/721R, 355C, 355L/430R, 355T, 358N/467N, 406G, 406L, 407V, 423E, 423E/463W/605K, 431T, 431V, 451G, 463W, 466H, 466M, 467N, 475C, 475H, 475L, 476V, 481S, 489R, 489R/494V, 489R/494V/659G, 489R/510R, 489R/658E, 494Y, 501V, 510M, 510R, 510R/748G, 519F, 519L, 530D, 530E, 530T, 530W, 533T, 534A, 534G, 538M, 543L, 543V, 543V/699S/814T, 557R, 557R/605K/699Q/721R, 560S, 597G, 597N, 597S, 605K, 621E, 621K, 621R, 659T, 666E, 666S, 666W, 694T, 696Q, 699G, 699Q, 699S, 721A, 721R, 722V, 748G, 748G/749K, 749G, 749K, 749S, 756L, 802C, 802R, 814K, and 814T, or combinations thereof, wherein the amino acid positions are relative to the reference sequence of SEQ ID NO: 2176.

In some embodiments, the engineered DNA polymerase comprises one or more substitutions or substitution sets selected from R45G, R45L, R45L/S621R, R45S, R45S/E65N/A243N/R268Q, R45S/E109H/A243N/A423E/Q543V/R605K/A699Q/S748G/V749K/E814T, R45S/E109H/R340T/R605K/E814T, R45S/S221D/A243N/R340T/A423E, R45S/A243N/E312Q/L331A/A451G, R45S/A243N/E312Q/L331A/P557R, R45S/A243N/E312Q/R340T/A423E/Q543A/E814T, R45S/A243N/E312Q/R605K, R45S/A243N/E814T, R45S/R268G/E312Q/R605K/S748G/V749K/E814T, R45S/R268Q/H489R/Q543V/P557R/S659T/E721R, R45S/R268Q/H489R/P557R/S748G/V749K, R45S/E312Q, R45S/E312Q/R340T/R605K, R45S/R340T/A423E/E814T, R45S/R340T/E475H/P557R/E814T, R45S/R340T/P557R, R45S/A423E, R45S/G431T, R45S/A451G, R45S/E475H/P557R/S748G/V749K, R45S/H489R/P557R/V749K, R45S/A699Q/S748G/V749K, R45V, K64H/E293D, E65C, E65K/H489R/Q543A, E65N, E65S, F81W, A94K, R102P, E109H, E109H/R340T/H489R/P510R/S659G, E109H/A423E, R118L, R118V, E120D, R151G, S162T, A194G, T211R, S221D, K233R, A243N, A243N/R268G, A243S, L245T, R252L, R258L, R268E, R268G, R268G/A423E/E814T, R268G/G431T/R605K/V749K, R268Q, R268Q/E312Q/L331A/R340T/A699Q, R268Q/E312Q/L331A/A423E/A451G/P557R/R605K, R268Q/R340T/R352Q/A423E/A451G, R268Q/A423E, R268Q/A423E/G431T/Q543A/P557R/R605K/E814T, R268Q/A423E/E814T, R269G, R269G/L658E, R269I, E272V, A280G, E283A, E283L, E283S, R284G, H292R, E298D, E298I, P300E, P300G, P300L, P300M, P300S, P300T, L301C, L301E, L301G, L301G/V538M/E546A/S621R/E814T, P302L/R340T/H489R/N494S/P510R/S748G/E814T, P302L/R340T/H489R/N494V/A525R, P302V/R340T/N494V, P302V/N494V, P302V/N494V/A525R, E304G/H489R, E312Q, E312Q/L331A/R605K/E814T, E312Q/A423E/G431T/A451G/H489R, E312Q/N494V/A525R/Q543V/S659G/S748G/E814T, P323W, E329M, E329S, L331A, L331S/R605K, L331V, R340L, R340T, R340T/H489R, R340T/H489R/N494V/Q543A/S659G/E814T, R340T/H489R/A699Q, A350C, A350G, A350Y, R352Q, R352Q/A451G/H489R/Q543V/P557R/E721R, F355C, F355L/Q430R, F355T, R358N/A467N, E406G, E406L, W407V, A423E, A423E/A463W/R605K, G431T, G431V, A451G, A463W, E466H, E466M, A467N, E475C, E475H, E475L, M476V, E481S, H489R, H489R/N494V, H489R/N494V/S659G, H489R/P510R, H489R/L658E, N494Y, R501V, P510M, P510R, P510R/S748G, G519F, G519L, A530D, A530E, A530T, A530W, E533T, V534A, V534G, V538M, Q543L, Q543V, Q543V/A699S/E814T, P557R, P557R/R605K/A699Q/E721R, A560S, T597G, T597N, T597S, R605K, S621E, S621K, S621R, S659T, L666E, L666S, L666W, P694T, E696Q, A699G, A699Q, A699S, E721A, E721R, E722V, S748G, S748G/V749K, V749G, V749K, V749S, M756L, K802C, K802R, E814K, and E814T, or combinations thereof, wherein the amino acid positions are relative to the reference sequence of SEQ ID NO: 2176.

In some embodiments, the engineered DNA polymerase comprises one or more substitutions or substitution sets selected from 45, 45/65/243/268, 45/221/243/340/423, 45/268/312/605/748/749/814, 45/268/489/557/748/749, 45/451, 45/475/557/748/749, 45/489/557/749, 45/621, 45/699/748/749, 65, 65/489/543, 81, 94, 102, 109, 109/340/489/510/659, 221, 227/443/756, 252, 268, 268/312/331/340/699, 268/312/331/423/451/557/605, 268/423/431/543/557/605/814, 269, 283, 300, 301, 301/538/546/621/814, 302/340/489/494/510/748/814, 302/340/489/494/525, 302/494/525, 304/489, 312, 312/494/525/543/659/748/814, 329, 331, 340, 340/489/699, 352, 358/467, 406, 431, 451, 463, 466, 475, 481, 489, 489/510, 489/658, 510, 510/748, 530, 543, 543/699/814, 557, 557/605/699/721, 560, 605, 659, 666, 696, 699, 721, 722, 748, 748/749, 749, 802, and 814, or combinations thereof, wherein the amino acid positions are relative to the reference sequence of SEQ ID NO: 2176.

In some embodiments, the engineered DNA polymerase comprises one or more substitutions or substitution sets selected from 45L/621R, 45S, 45S/65N/243N/268Q, 45S/221D/243N/340T/423E, 45S/268G/312Q/605K/748G/749K/814T, 45S/268Q/489R/557R/748G/749K, 45S/451G, 45S/475H/557R/748G/749K, 45S/489R/557R/749K, 45S/699Q/748G/749K, 65K/489R/543A, 65N, 81W, 94K, 102P, 109H, 109H/340T/489R/510R/659G, 221D, 227E/443D/756R, 252L, 268G, 268Q, 268Q/312Q/331A/340T/699Q, 268Q/312Q/331A/423E/451G/557R/605K, 268Q/423E/431T/543A/557R/605K/814T, 269G, 283L, 300M, 300S, 300T, 301G, 301G/538M/546A/621R/814T, 302L/340T/489R/494S/510R/748G/814T, 302L/340T/489R/494V/525R, 302V/494V/525R, 304G/489R, 312Q, 312Q/494V/525R/543V/659G/748G/814T, 329S, 331A, 340T, 340T/489R/699Q, 352Q, 358N/467N, 406L, 431T, 451G, 463W, 466M, 475C, 475H, 481S, 489R, 489R/510R, 489R/658E, 510R, 510R/748G, 530D, 530E, 530T, 543V, 543V/699S/814T, 557R, 557R/605K/699Q/721R, 560S, 605K, 659T, 666E, 666S, 666W, 696Q, 699Q, 721R, 722V, 748G, 748G/749K, 749K, 802C, and 814T, or combinations thereof, wherein the amino acid positions are relative to the reference sequence of SEQ ID NO: 2176.

In some embodiments, the engineered DNA polymerase comprises one or more substitutions or substitution sets selected from R45L/S621R, R45S, R45S/E65N/A243N/R268Q, R45S/S221D/A243N/R340T/A423E, R45S/R268G/E312Q/R605K/S748G/V749K/E814T, R45S/R268Q/H489R/P557R/S748G/V749K, R45S/A451G, R45S/E475H/P557R/S748G/V749K, R45S/H489R/P557R/V749K, R45S/A699Q/S748G/V749K, E65K/H489R/Q543A, E65N, F81W, A94K, R102P, E109H, E109H/R340T/H489R/P510R/S659G, S221D, K227E/E443D/M756R, R252L, R268G, R268Q, R268Q/E312Q/L331A/R340T/A699Q, R268Q/E312Q/L331A/A423E/A451G/P557R/R605K, R268Q/A423E/G431T/Q543A/P557R/R605K/E814T, R269G, E283L, P300M, P300S, P300T, L301G, L301G/V538M/E546A/S621R/E814T, P302L/R340T/H489R/N494S/P510R/S748G/E814T, P302L/R340T/H489R/N494V/A525R, P302V/N494V/A525R, E304G/H489R, E312Q, E312Q/N494V/A525R/Q543V/S659G/S748G/E814T, E329S, L331A, R340T, R340T/H489R/A699Q, R352Q, R358N/A467N, E406L, G431T, A451G, A463W, E466M, E475C, E475H, E481S, H489R, H489R/P510R, H489R/L658E, P510R, P510R/S748G, A530D, A530E, A530T, Q543V, Q543V/A699S/E814T, P557R, P557R/R605K/A699Q/E721R, A560S, R605K, S659T, L666E, L666S, L666W, E696Q, A699Q, E721R, E722V, S748G, S748G/V749K, V749K, K802C, and E814T, or combinations thereof, wherein the amino acid positions are relative to the reference sequence of SEQ ID NO: 2176.

In some embodiments, the engineered DNA polymerase comprises one or more substitutions or substitution sets selected from 45/65/243/268, 45/268/312/605/748/749/814, 45/268/489/557/748/749, 45/451, 45/475/557/748/749, 45/489/557/749, 45/621, 45/699/748/749, 57, 65, 65/489/543, 81, 94, 109, 221, 243, 268, 268/312/331/340/699, 268/312/331/423/451/557/605, 268/423/431/543/557/605/814, 269, 300, 301, 302/340/489/494/510/748/814, 302/494/525, 304/489, 312, 331, 331/605, 340, 340/489/699, 352, 358/467, 406, 423, 431, 451, 463, 466, 475, 481, 489, 489/510, 489/658, 494, 510, 510/748, 530, 543, 543/699/814, 557, 557/605/699/721, 560, 597, 605, 659, 666, 696, 699, 721, 748, 748/749, 749, 802, and 814, or combinations thereof, wherein the amino acid positions are relative to the reference sequence of SEQ ID NO: 2176.

In some embodiments, the engineered DNA polymerase comprises one or more substitutions or substitution sets selected from 45L/621R, 45S/65N/243N/268Q, 45S/268G/312Q/605K/748G/749K/814T, 45S/268Q/489R/557R/748G/749K, 45S/451G, 45S/475H/557R/748G/749K, 45S/489R/557R/749K, 45S/699Q/748G/749K, 57R, 65K/489R/543A, 65N, 81W, 94K, 109H, 221D, 243N, 268G, 268Q, 268Q/312Q/331A/340T/699Q, 268Q/312Q/331A/423E/451G/557R/605K, 268Q/423E/431T/543A/557R/605K/814T, 269G, 300S, 300T, 301G, 302L/340T/489R/494S/510R/748G/814T, 302V/494V/525R, 304G/489R, 312Q, 331A, 331S/605K, 340L, 340T, 340T/489R/699Q, 352Q, 358N/467N, 406L, 423E, 431T, 451G, 463W, 466M, 475C, 475H, 481S, 489R, 489R/510R, 489R/658E, 494S, 510R, 510R/748G, 530D, 530E, 530T, 543V, 543V/699S/814T, 557R, 557R/605K/699Q/721R, 560S, 597G, 597N, 605K, 659T, 666E, 666S, 666W, 696Q, 699Q, 721A, 721R, 748G, 748G/749K, 749K, 802C, 802R, and 814, or combinations thereof, wherein the amino acid positions are relative to the reference sequence of SEQ ID NO: 2176.

In some embodiments, the engineered DNA polymerase comprises one or more substitutions or substitution sets selected from R45L/S621R, R45S/E65N/A243N/R268Q, R45S/R268G/E312Q/R605K/S748G/V749K/E814T, R45S/R268Q/H489R/P557R/S748G/V749K, R45S/A451G, R45S/E475H/P557R/S748G/V749K, R45S/H489R/P557R/V749K, R45S/A699Q/S748G/V749K, K57R, E65K/H489R/Q543A, E65N, F81W, A94K, E109H, S221D, A243N, R268G, R268Q, R268Q/E312Q/L331A/R340T/A699Q, R268Q/E312Q/L331A/A423E/A451G/P557R/R605K, R268Q/A423E/G431T/Q543A/P557R/R605K/E814T, R269G, P300S, P300T, L301G, P302L/R340T/H489R/N494S/P510R/S748G/E814T, P302V/N494V/A525R, E304G/H489R, E312Q, L331A, L331S/R605K, R340L, R340T, R340T/H489R/A699Q, R352Q, R358N/A467N, E406L, A423E, G431T, A451G, A463W, E466M, E475C, E475H, E481S, H489R, H489R/P510R, H489R/L658E, N494S, P510R, P510R/S748G, A530D, A530E, A530T, Q543V, Q543V/A699S/E814T, P557R, P557R/R605K/A699Q/E721R, A560S, T597G, T597N, R605K, S659T, L666E, L666S, L666W, E696Q, A699Q, E721A, E721R, S748G, S748G/V749K, V749K, K802C, K802R, and 814T, or combinations thereof, wherein the amino acid positions are relative to the reference sequence of SEQ ID NO: 2176.

The present invention also provides engineered DNA polymerases or functional fragments thereof, comprising a polypeptide sequence having at least 75% or more sequence identity to a reference sequence comprising residues 12 to 841 of SEQ ID NO: 2310, or to a reference sequence of SEQ ID NO: 2310, wherein the polypeptide sequence comprises one or more substitutions relative to the reference sequence comprising residues 12 to 841 of SEQ ID NO: 2310, or to the reference sequence of SEQ ID NO: 2310.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2310 or to the reference sequence of SEQ ID NO: 2310.

In some embodiments, the engineered DNA polymerase comprises one or more substitutions or substitution sets selected from 18/268/302/312/605/659/699, 45/227/268/423/489/510/605, 45/227/302/423/510/605/621/699, 45/227/312/423/525/605/699, 45/268/302/423/494/621/699, 45/268/443/525/696, 45/331/489, 45/331/621, 45/340/423, 45/494/525/621/696/699, 45/605, 45/696/699, 65/685, 227, 227/268/340/451, 227/268/423/621, 227/302, 227/312/489/494/621, 227/331, 227/331/423/605, 227/331/605/659, 227/340/423/605, 227/340/621, 227/423, 227/423/451, 227/451/525/621/659/699/721, 227/525, 227/543/659/699, 227/605/721, 227/699, 268/302/340/358/605/699, 268/302/423/605, 268/340, 268/340/423/443/525/621/659, 268/340/423/621, 268/340/525/605, 268/423, 268/423/478/489/525/605/699, 268/423/489/494/605/659, 268/423/510/605, 268/423/605/699, 268/423/699, 268/467, 268/467/489, 268/467/489/510/699, 268/467/489/699, 268/467/489/699/814, 268/467/510/699/814, 268/489/494/605/699, 268/489/699, 268/510, 268/510/699/814, 268/525, 268/525/605, 268/543, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/451, 331/525/605/699, 331/605/699, 340/451/494/525, 358/467/489/699, 358/467/699, 358/467/699/814, 423/443/451/494/510, 423/451/605/659/699, 423/489/494/529/605/621/696/699, 423/494, 423/525, 423/605/621, 451/510, 467, 467/489, 467/489/510/543/699/814, 467/489/699, 467/489/699/814, 467/489/814, 467/510/699, 467/510/699/814, 467/510/814, 467/699, 484, 489, 489/494/605/721, 489/510/525/605/699, 489/699, 489/699/814, 494/605/699, 494/659, 494/699, 498, 503, 510, 510/605/659, 510/699, 510/699/814, 524, 526, 527, 531, 540, 542, 543/699, 543/699/814, 547, 569, 577/614, 605, 605/621/659/696/699, 605/621/696/699, 621, 659/699, 673, 684, 685, 685/714, 699, 699/814, 702, and 716, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/227E/312Q/423E/525R/605K/699Q, 45L/268Q/302L/423E/494S/621R/699S, 45L/268Q/443D/525R/696Q, 45L/331A/489R, 45L/331A/621R, 45L/340T/423E, 45L/494V/525R/621R/696Q/699Q, 45L/605K, 45L/696Q/699Q, 65K/685R, 227E, 227E/268G/423E/621R, 227E/268Q/340T/451G, 227E/302L, 227E/312Q/489R/494S/621R, 227E/331A/423E/605K, 227E/331S, 227E/331S/605K/659G, 227E/340T/423E/605K, 227E/340T/621R, 227E/423E, 227E/423E/451G, 227E/451G/525R/621R/659G/699Q/721R, 227E/525R, 227E/543V/659G/699S, 227E/605K/721R, 227E/699S, 268G/302L/423E/605K, 268G/340T, 268G/423E/478H/489R/525R/605K/699Q, 268G/423E/510R/605K, 268G/423E/699Q, 268G/621R, 268Q/302L/340T/358N/605K/699Q, 268Q/340T/423E/443D/525R/621R/659G, 268Q/340T/423E/621R, 268Q/340T/525R/605K, 268Q/423E, 268Q/423E/489R/494S/605K/659G, 268Q/423E/605K/699S, 268Q/467N, 268Q/467N/489R, 268Q/467N/489R/510R/699Q, 268Q/467N/489R/699Q, 268Q/467N/489R/699S/814T, 268Q/467N/510R/699S/814T, 268Q/489R/494S/605K/699Q, 268Q/489R/699Q, 268Q/510R, 268Q/510R/699Q/814T, 268Q/525R, 268Q/525R/605K, 268Q/543V, 268Q/543V/699Q, 268Q/605K/621R/721R, 268Q/699Q, 268Q/699S, 302L/423E, 302L/423E/605K/621R, 302L/543V/621R/696Q/699Q, 302L/605K/621R/721R, 312Q/331A/451G/621R/696Q/699Q, 312Q/423E/621R, 312Q/510R/605K, 331A/423E/527F, 331A/525R/605K/699Q, 331A/605K/699Q, 331S/423E, 331S/451G, 340T/451G/494V/525R, 358N/467N/489R/699Q, 358N/467N/699Q, 358N/467N/699Q/814T, 423E/443D/451G/494S/510R, 423E/451G/605K/659G/699Q, 423E/489R/494S/529D/605K/621R/696Q/699Q, 423E/494S, 423E/525R, 423E/605K/621R, 451G/510R, 467N, 467N/489R, 467N/489R/510R/543V/699S/814T, 467N/489R/699Q, 467N/489R/699S/814T, 467N/489R/814T, 467N/510R/699Q, 467N/510R/699Q/814T, 467N/510R/699S/814T, 467N/510R/814T, 467N/699Q, 467N/699S, 484V, 489R, 489R/494S/605K/721R, 489R/510R/525R/605K/699Q, 489R/699Q, 489R/699Q/814T, 489R/699S, 494S/659G, 494S/699S, 494V/605K/699Q, 498M, 498T, 5031, 510R, 510R/605K/659G, 510R/699Q, 510R/699Q/814T, 510R/699S/814T, 524D, 524Q, 526Q, 527R, 531V, 540S, 542R, 543V/699Q, 543V/699Q/814T, 543V/699S/814T, 547M, 569V, 577S/614C, 605K, 605K/621R/659G/696Q/699S, 605K/621R/696Q/699S, 621R, 659G/699Q, 673K, 684P, 685K, 685K/714T, 685R, 699Q, 699Q/814T, 702K, and 716R, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/K227E/E312Q/A423E/A525R/R605K/A699Q, S45L/R268Q/P302L/A423E/N494S/S621R/A699S, S45L/R268Q/E443D/A525R/E696Q, S45L/L331A/H489R, S45L/L331A/S621R, S45L/R340T/A423E, S45L/N494V/A525R/S621R/E696Q/A699Q, S45L/R605K, S45L/E696Q/A699Q, E65K/F685R, K227E, K227E/R268G/A423E/S621R, K227E/R268Q/R340T/A451G, K227E/P302L, K227E/E312Q/H489R/N494S/S621R, K227E/L331A/A423E/R605K, K227E/L331S, K227E/L331S/R605K/S659G, K227E/R340T/A423E/R605K, K227E/R340T/S621R, K227E/A423E, K227E/A423E/A451G, K227E/A451G/A525R/S621R/S659G/A699Q/E721R, K227E/A525R, K227E/Q543V/S659G/A699S, K227E/R605K/E721R, K227E/A699S, R268G/P302L/A423E/R605K, R268G/R340T, R268G/A423E/R478H/H489R/A525R/R605K/A699Q, R268G/A423E/P510R/R605K, R268G/A423E/A699Q, R268G/S621R, R268Q/P302L/R340T/R358N/R605K/A699Q, R268Q/R340T/A423E/E443D/A525R/S621R/S659G, R268Q/R340T/A423E/S621R, R268Q/R340T/A525R/R605K, R268Q/A423E, R268Q/A423E/H489R/N494S/R605K/S659G, R268Q/A423E/R605K/A699S, R268Q/A467N, R268Q/A467N/H489R, R268Q/A467N/H489R/P510R/A699Q, R268Q/A467N/H489R/A699Q, R268Q/A467N/H489R/A699S/E814T, R268Q/A467N/P510R/A699S/E814T, R268Q/H489R/N494S/R605K/A699Q, R268Q/H489R/A699Q, R268Q/P510R, R268Q/P510R/A699Q/E814T, R268Q/A525R, R268Q/A525R/R605K, R268Q/Q543V, R268Q/Q543V/A699Q, R268Q/R605K/S621R/E721R, R268Q/A699Q, R268Q/A699S, P302L/A423E, P302L/A423E/R605K/S621R, P302L/Q543V/S621R/E696Q/A699Q, P302L/R605K/S621R/E721R, E312Q/L331A/A451G/S621R/E696Q/A699Q, E312Q/A423E/S621R, E312Q/P510R/R605K, L331A/A423E/V527F, L331A/A525R/R605K/A699Q, L331A/R605K/A699Q, L331S/A423E, L331S/A451G, R340T/A451G/N494V/A525R, R358N/A467N/H489R/A699Q, R358N/A467N/A699Q, R358N/A467N/A699Q/E814T, A423E/E443D/A451G/N494S/P510R, A423E/A451G/R605K/S659G/A699Q, A423E/H489R/N494S/E529D/R605K/S621R/E696Q/A699Q, A423E/N494S, A423E/A525R, A423E/R605K/S621R, A451G/P510R, A467N, A467N/H489R, A467N/H489R/P510R/Q543V/A699S/E814T, A467N/H489R/A699Q, A467N/H489R/A699S/E814T, A467N/H489R/E814T, A467N/P510R/A699Q, A467N/P510R/A699Q/E814T, A467N/P510R/A699S/E814T, A467N/P510R/E814T, A467N/A699Q, A467N/A699S, F484V, H489R, H489R/N494S/R605K/E721R, H489R/P510R/A525R/R605K/A699Q, H489R/A699Q, H489R/A699Q/E814T, H489R/A699S, N494S/S659G, N494S/A699S, N494V/R605K/A699Q, Q498M, Q498T, L503I, P510R, P510R/R605K/S659G, P510R/A699Q, P510R/A699Q/E814T, P510R/A699S/E814T, S524D, S524Q, A526Q, V527R, L531V, K540S, L542R, Q543V/A699Q, Q543V/A699Q/E814T, Q543V/A699S/E814T, L547M, L569V, A577S/R614C, R605K, R605K/S621R/S659G/E696Q/A699S, R605K/S621R/E696Q/A699S, S621R, S659G/A699Q, T673K, A684P, F685K, F685K/A714T, F685R, A699Q, A699Q/E814T, I702K, and I716R, or combinations thereof, wherein the amino acid positions are relative to the reference sequence of SEQ ID NO: 2310.

In some embodiments, the engineered DNA polymerase comprises one or more substitutions or substitution sets selected from 18/268/302/312/605/659/699, 45/227/268/423/489/510/605, 45/227/302/423/510/605/621/699, 45/227/312/423/525/605/699, 45/268/302/423/494/621/699, 45/494/525/621/696/699, 227, 227/268/340/451, 227/268/423/621, 227/302, 227/312/489/494/621, 227/331, 227/331/423/605, 227/331/605/659, 227/340/423/605, 227/340/621, 227/423, 227/543/659/699, 227/605/721, 268, 268/302, 268/302/423/605, 268/340/423/443/525/621/659, 268/423/510/605, 268/423/605/699, 268/467, 268/467/489, 268/467/489/510/699, 268/467/489/699, 268/489/699, 268/510, 268/510/699/814, 268/525/605, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/605, 331/605/699, 340/451/494/525, 358/467/489/699, 358/467/699/814, 423, 423/443/451/494/510, 423/489/494/529/605/621/696/699, 423/605/621, 451/510, 467/489, 467/489/510/543/699/814, 467/489/699, 467/489/814, 467/510/699, 467/510/699/814, 467/699, 467/699/814, 484, 489, 489/510/525/605/699, 489/699, 489/699/814, 494/605/699, 498, 503, 510, 510/605/659, 510/699, 510/699/814, 524, 526, 543/699, 543/699/814, 605/621/659/696/699, 605/621/696/699, 621, and 699/814, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/227E/312Q/423E/525R/605K/699Q, 45L/268Q/302L/423E/494S/621R/699S, 45L/494V/525R/621R/696Q/699Q, 227E, 227E/268G/423E/621R, 227E/268Q/340T/451G, 227E/302L, 227E/312Q/489R/494S/621R, 227E/331A/423E/605K, 227E/331S, 227E/331S/605K/659G, 227E/340T/423E/605K, 227E/340T/621R, 227E/423E, 227E/543V/659G/699S, 227E/605K/721R, 268G/302L/423E/605K, 268G/423E/510R/605K, 268G/621R, 268Q, 268Q/302L, 268Q/340T/423E/443D/525R/621R/659G, 268Q/423E/605K/699S, 268Q/467N, 268Q/467N/489R, 268Q/467N/489R/510R/699Q, 268Q/467N/489R/699Q, 268Q/489R/699Q, 268Q/510R, 268Q/510R/699Q/814T, 268Q/525R/605K, 268Q/543V/699Q, 268Q/605K/621R/721R, 268Q/699Q, 302L/423E, 302L/423E/605K/621R, 302L/543V/621R/696Q/699Q, 302L/605K/621R/721R, 312Q/331A/451G/621R/696Q/699Q, 312Q/423E/621R, 312Q/510R/605K, 331A/423E/527F, 331A/605K, 331A/605K/699Q, 331S/423E, 331S/605K, 340T/451G/494V/525R, 358N/467N/489R/699Q, 358N/467N/699Q/814T, 423E, 423E/443D/451G/494S/510R, 423E/489R/494S/529D/605K/621R/696Q/699Q, 423E/605K/621R, 451G/510R, 467N/489R, 467N/489R/510R/543V/699S/814T, 467N/489R/699Q, 467N/489R/814T, 467N/510R/699Q, 467N/510R/699Q/814T, 467N/510R/699S/814T, 467N/699Q, 467N/699Q/814T, 467N/699S, 484V, 489R, 489R/510R/525R/605K/699Q, 489R/699Q, 489R/699Q/814T, 494V/605K/699Q, 498M, 498T, 5031, 510R, 510R/605K/659G, 510R/699Q, 510R/699Q/814T, 510R/699S/814T, 524D, 524Q, 526Q, 543V/699Q, 543V/699Q/814T, 605K/621R/659G/696Q/699S, 605K/621R/696Q/699S, 621R, and 699Q/814T, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/K227E/E312Q/A423E/A525R/R605K/A699Q, S45L/R268Q/P302L/A423E/N494S/S621R/A699S, S45L/N494V/A525R/S621R/E696Q/A699Q, K227E, K227E/R268G/A423E/S621R, K227E/R268Q/R340T/A451G, K227E/P302L, K227E/E312Q/H489R/N494S/S621R, K227E/L331A/A423E/R605K, K227E/L331S, K227E/L331S/R605K/S659G, K227E/R340T/A423E/R605K, K227E/R340T/S621R, K227E/A423E, K227E/Q543V/S659G/A699S, K227E/R605K/E721R, R268G/P302L/A423E/R605K, R268G/A423E/P510R/R605K, R268G/S621R, R268Q, R268Q/P302L, R268Q/R340T/A423E/E443D/A525R/S621R/S659G, R268Q/A423E/R605K/A699S, R268Q/A467N, R268Q/A467N/H489R, R268Q/A467N/H489R/P510R/A699Q, R268Q/A467N/H489R/A699Q, R268Q/H489R/A699Q, R268Q/P510R, R268Q/P510R/A699Q/E814T, R268Q/A525R/R605K, R268Q/Q543V/A699Q, R268Q/R605K/S621R/E721R, R268Q/A699Q, P302L/A423E, P302L/A423E/R605K/S621R, P302L/Q543V/S621R/E696Q/A699Q, P302L/R605K/S621R/E721R, E312Q/L331A/A451G/S621R/E696Q/A699Q, E312Q/A423E/S621R, E312Q/P510R/R605K, L331A/A423E/V527F, L331A/R605K, L331A/R605K/A699Q, L331S/A423E, L331S/R605K, R340T/A451G/N494V/A525R, R358N/A467N/H489R/A699Q, R358N/A467N/A699Q/E814T, A423E, A423E/E443D/A451G/N494S/P510R, A423E/H489R/N494S/E529D/R605K/S621R/E696Q/A699Q, A423E/R605K/S621R, A451G/P510R, A467N/H489R, A467N/H489R/P510R/Q543V/A699S/E814T, A467N/H489R/A699Q, A467N/H489R/E814T, A467N/P510R/A699Q, A467N/P510R/A699Q/E814T, A467N/P510R/A699S/E814T, A467N/A699Q, A467N/A699Q/E814T, A467N/A699S, F484V, H489R, H489R/P510R/A525R/R605K/A699Q, H489R/A699Q, H489R/A699Q/E814T, N494V/R605K/A699Q, Q498M, Q498T, L503I, P510R, P510R/R605K/S659G, P510R/A699Q, P510R/A699Q/E814T, P510R/A699S/E814T, S524D, S524Q, A526Q, Q543V/A699Q, Q543V/A699Q/E814T, R605K/S621R/S659G/E696Q/A699S, R605K/S621R/E696Q/A699S, S621R, and A699Q/E814T, or combinations thereof, wherein the amino acid positions are relative to the reference sequence of SEQ ID NO: 2310.

In some embodiments, the engineered DNA polymerase comprises one or more substitutions or substitution sets selected from 18/268/302/312/605/659/699, 45/227/268/423/489/510/605, 45/227/302/423/510/605/621/699, 45/494/525/621/696/699, 227/331/605/659, 227/605/721, 268, 268/302/423/605, 268/423/605/699, 268/467, 268/467/489, 268/467/489/510/699, 268/467/489/699, 268/489/699, 268/510, 268/605/621/721, 302/543/621/696/699, 312/331/451/621/696/699, 312/510/605, 331/423/527, 340/451/494/525, 423/489/494/529/605/621/696/699, 423/605/621, 467/489/510/543/699/814, 467/489/699, 467/489/814, 467/510/699/814, 489/510/525/605/699, 489/699, 489/699/814, 494/605/699, 510, 510/699/814, 543/699, and 543/699/814, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/494V/525R/621R/696Q/699Q, 227E/331S/605K/659G, 227E/605K/721R, 268G/302L/423E/605K, 268Q, 268Q/423E/605K/699S, 268Q/467N, 268Q/467N/489R, 268Q/467N/489R/510R/699Q, 268Q/467N/489R/699Q, 268Q/489R/699Q, 268Q/510R, 268Q/605K/621R/721R, 302L/543V/621R/696Q/699Q, 312Q/331A/451G/621R/696Q/699Q, 312Q/510R/605K, 331A/423E/527F, 340T/451G/494V/525R, 423E/489R/494S/529D/605K/621R/696Q/699Q, 423E/605K/621R, 467N/489R/510R/543V/699S/814T, 467N/489R/699Q, 467N/489R/814T, 467N/510R/699Q/814T, 467N/510R/699S/814T, 489R/510R/525R/605K/699Q, 489R/699Q, 489R/699Q/814T, 494V/605K/699Q, 510R, 510R/699Q/814T, 510R/699S/814T, 543V/699Q, and 543V/699Q/814T, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/N494V/A525R/S621R/E696Q/A699Q, K227E/L331S/R605K/S659G, K227E/R605K/E721R, R268G/P302L/A423E/R605K, R268Q, R268Q/A423E/R605K/A699S, R268Q/A467N, R268Q/A467N/H489R, R268Q/A467N/H489R/P510R/A699Q, R268Q/A467N/H489R/A699Q, R268Q/H489R/A699Q, R268Q/P510R, R268Q/R605K/S621R/E721R, P302L/Q543V/S621R/E696Q/A699Q, E312Q/L331A/A451G/S621R/E696Q/A699Q, E312Q/P510R/R605K, L331A/A423E/V527F, R340T/A451G/N494V/A525R, A423E/H489R/N494S/E529D/R605K/S621R/E696Q/A699Q, A423E/R605K/S621R, A467N/H489R/P510R/Q543V/A699S/E814T, A467N/H489R/A699Q, A467N/H489R/E814T, A467N/P510R/A699Q/E814T, A467N/P510R/A699S/E814T, H489R/P510R/A525R/R605K/A699Q, H489R/A699Q, H489R/A699Q/E814T, N494V/R605K/A699Q, P510R, P510R/A699Q/E814T, P510R/A699S/E814T, Q543V/A699Q, and Q543V/A699Q/E814T, or combinations thereof, wherein the amino acid positions are relative to the reference sequence of SEQ ID NO: 2310.

In some embodiments, the engineered DNA polymerase comprises one or more substitutions or substitution sets selected from 18/268/302/312/605/659/699, 45/227/268/423/489/510/605, 45/227/302/423/510/605/621/699, 65/685, 227/331/605/659, 227/605/721, 268, 268/302/423/605, 268/423/605/699, 268/467, 268/467/489, 268/467/489/699, 268/489/699, 268/510, 268/605/621/721, 302/543/621/696/699, 312/331/451/621/696/699, 312/510/605, 423/605/621, 467/489/510/543/699/814, 467/489/699, 467/489/814, 467/510/699/814, 489/699, 489/699/814, 510, 510/699/814, 542, 543/699, 543/699/814, 685, and 685/714, or combinations thereof, wherein the amino acid positions are relative to the reference sequence of SEQ ID NO: 2310.

In some embodiments, the engineered DNA polymerase comprises one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 65K/685R, 227E/331S/605K/659G, 227E/605K/721R, 268G/302L/423E/605K, 268Q, 268Q/423E/605K/699S, 268Q/467N, 268Q/467N/489R, 268Q/467N/489R/699Q, 268Q/489R/699Q, 268Q/510R, 268Q/605K/621R/721R, 302L/543V/621R/696Q/699Q, 312Q/331A/451G/621R/696Q/699Q, 312Q/510R/605K, 423E/605K/621R, 467N/489R/510R/543V/699S/814T, 467N/489R/699Q, 467N/489R/814T, 467N/510R/699Q/814T, 467N/510R/699S/814T, 489R/699Q, 489R/699Q/814T, 510R, 510R/699S/814T, 542R, 543V/699Q, 543V/699Q/814T, 685K, 685K/714T, and 685, or combinations thereof, wherein the amino acid positions are relative to the reference sequence of SEQ ID NO: 2310.

In some embodiments, the engineered DNA polymerase comprises one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, E65K/F685R, K227E/L331S/R605K/S659G, K227E/R605K/E721R, R268G/P302L/A423E/R605K, R268Q, R268Q/A423E/R605K/A699S, R268Q/A467N, R268Q/A467N/H489R, R268Q/A467N/H489R/A699Q, R268Q/H489R/A699Q, R268Q/P510R, R268Q/R605K/S621R/E721R, P302L/Q543V/S621R/E696Q/A699Q, E312Q/L331A/A451G/S621R/E696Q/A699Q, E312Q/P510R/R605K, A423E/R605K/S621R, A467N/H489R/P510R/Q543V/A699S/E814T, A467N/H489R/A699Q, A467N/H489R/E814T, A467N/P510R/A699Q/E814T, A467N/P510R/A699S/E814T, H489R/A699Q, H489R/A699Q/E814T, P510R, P510R/A699S/E814T, L542R, Q543V/A699Q, Q543V/A699Q/E814T, F685K, F685K/A714T, and F685R, or combinations thereof, wherein the amino acid positions are relative to the reference sequence of SEQ ID NO: 2310.

The present invention also provides engineered DNA polymerases or functional fragments thereof, comprising a polypeptide sequence having at least 75% or more sequence identity to a reference sequence comprising residues 12 to 841 of SEQ ID NO: 2994, or to a reference sequence of SEQ ID NO: 2994, wherein the polypeptide sequence comprises one or more substitutions relative to the reference sequence comprising residues 12 to 841 of SEQ ID NO: 2994, or to the reference sequence of SEQ ID NO: 2994.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2994 or to the reference sequence of SEQ ID NO: 2994.

In some embodiments, the engineered DNA polymerase comprises one or more substitutions or substitution sets at amino acid positions selected from 4, 6, 10, 11, 12, 14, 14/50, 14/50/56/598/725, 14/50/598/695, 14/95, 14/95/501/642, 14/139/249/433, 14/139/725, 14/167, 14/167/197/433, 14/167/197/501/725, 14/167/197/642/695, 14/167/249/433/598/642, 14/167/249/501/598/642, 14/167/249/598/695/725, 14/167/249/642, 14/167/642, 14/167/695, 14/167/725, 14/197/249/642, 14/197/642, 14/249/433/725, 14/249/598, 14/249/642, 14/249/725, 14/433/598, 14/501, 14/598, 14/598/695, 14/598/695/725, 14/642/725, 18, 18/302, 18/302/531/673/684, 18/302/547, 18/484/673, 18/484/673/684, 18/524/547, 18/547, 18/684, 29, 45/268/299/302/321/621/659, 45/268/302/621/721, 45/376/385/408/494/525/621/696, 45/423, 48, 50, 50/433/598, 50/501, 50/598, 50/598/695, 50/642, 53, 76, 77, 91, 95/167/197/598/642/695, 95/167/197/598/695/725, 95/249/433/642, 95/249/598, 95/598, 99, 105, 122, 139/167, 139/167/197/433/501/642, 139/433/598, 141, 167, 167/197, 167/197/642/695/725, 167/642, 167/725, 177, 197/642/725, 197/695, 202, 204, 209, 217, 227, 239, 247, 249/695, 249/725, 266, 268, 268/302/451/494/621, 270, 291, 291/684, 299, 299/474, 302, 302/423/451/494/621, 302/423/621, 302/451/494, 313, 346, 349, 356, 361, 370, 374, 377, 394, 404, 423, 423/451/621/696, 423/494/525, 426, 426/798, 427, 433/501/725, 436, 439/509/789, 440, 444, 445, 445/446, 446/811, 448, 448/793, 459, 461, 482, 484, 484/684, 485, 489, 494, 498/684, 509, 514, 517, 525/529/621, 533, 543/621/721, 547, 565, 574, 596, 598, 598/642, 598/642/695, 598/695, 598/725, 601, 610, 621, 621/659, 636, 642, 642/692, 659, 662, 673, 683, 684, 688, 689, 690, 695, 725, 741, 744, 744/793, 751, 801, 810, 818, 830, and 832, or combinations thereof, wherein the amino acid positions are relative to the reference sequence of SEQ ID NO: 2994.

In some embodiments, the engineered DNA polymerase comprises one or more substitutions or substitution sets selected from 4A, 4F, 4G, 4R, 4T, 4V, 6F, 6G, 6N, 6R, 6V, 101, 10Q, 10R, 11E, 11L, 11R, 12D, 12W, 14E, 14E/50H, 14E/50H/56V/598K/725H, 14E/50H/598K/695K, 14E/95D, 14E/95D/501K/642E, 14E/139D/249E/433K, 14E/139D/725H, 14E/167D, 14E/167D/197K/433K, 14E/167D/197K/501K/725H, 14E/167D/197K/642E/695K, 14E/167D/249E/433K/598K/642E, 14E/167D/249E/501K/598K/642E, 14E/167D/249E/598K/695K/725H, 14E/167D/249E/642E, 14E/167D/642E, 14E/167D/695K, 14E/167D/725H, 14E/197K/249E/642E, 14E/197K/642E, 14E/249E/433K/725H, 14E/249E/598K, 14E/249E/642E, 14E/249E/725H, 14E/433K/598K, 14E/501K, 14E/598K, 14E/598K/695K, 14E/598K/695K/725H, 14E/642E/725H, 18H, 18H/302V, 18H/302V/531V/673K/684P, 18H/302V/547M, 18H/484V/673K, 18H/484V/673K/684P, 18H/524D/547M, 18H/547M, 18H/684P, 29M, 29N, 29V, 29W, 45L/268G/299N/302L/321T/621R/659G, 45L/268G/302L/621R/721R, 45L/376F/385F/408A/494V/525R/621R/696Q, 45L/423E, 48M, 48V, 50H, 50H/433K/598K, 50H/501K, 50H/598K, 50H/598K/695K, 50H/642E, 53A, 53Q, 53W, 76L, 76S, 76W, 76Y, 77H, 77M, 77R, 77S, 91C, 91R, 91S, 91W, 95D/167D/197K/598K/642E/695K, 95D/167D/197K/598K/695K/725H, 95D/249E/433K/642E, 95D/249E/598K, 95D/598K, 99G, 99L, 99P, 99S, 99T, 105E, 105F, 105G, 105L, 105S, 105T, 122G, 122I, 122L, 122N, 122R, 122S, 139D/167D, 139D/167D/197K/433K/501K/642E, 139D/433K/598K, 141C, 167D, 167D/197K, 167D/197K/642E/695K/725H, 167D/642E, 167D/725H, 177C, 177L, 177Q, 177R, 197K/642E/725H, 197K/695K, 202G, 204L, 209G, 209M, 209N, 209V, 217G, 217R, 217T, 227M, 227S, 239I, 239L, 239W, 239Y, 247A, 247G, 249E/695K, 249E/725H, 266E, 268G, 268G/302V/451G/494S/621R, 268H, 268S, 270P, 291Q, 291Q/684P, 299E, 299F, 299G, 299K, 299M, 299P, 299R/474V, 299V, 302V, 302V/423E/451G/494S/621R, 302V/423E/621R, 302V/451G/494S, 313F, 313S, 313T, 313V, 346H, 346M, 346P, 346R, 346T, 349N, 349P, 349R, 349T, 349V, 349W, 356A, 356G, 356I, 356Q, 356R, 356V, 361G, 370D, 370T, 370Y, 374G, 377G, 377L, 394C, 394D, 394G, 394M, 394R, 404C, 404K, 404R, 404S, 404V, 423E, 423E/451G/621R/696Q, 423E/494V/525R, 423F, 423G, 423R, 423S, 423V, 426A, 426G, 426H, 426Q, 426R/798F, 426V, 426W, 427A, 427C, 427E, 427K, 427L, 427M, 427R, 427S, 427T, 427W, 433K/501K/725H, 436E, 436G, 436H, 436K, 436S, 436W, 436Y, 439F/509V/7891, 440C, 440G, 440W, 444T, 444V, 445G/446V, 445H, 446M/811I, 448G, 448K, 448Q, 448S, 448T, 448V/793P, 459F, 459L, 459R, 461A, 482A, 484V, 484V/684P, 485C, 485D, 485F, 485M, 485N, 485Q, 485S, 485T, 489S, 489W, 494V, 498M/684P, 509A, 514R, 514S, 517I, 517N, 517R, 517S, 517T, 525R/529D/621R, 533C, 533R, 543V/621R/721R, 547M, 565C, 565R, 565S, 565T, 574L, 574Q, 596A, 596V, 598K, 598K/642E, 598K/642E/695K, 598K/695K, 598K/725H, 601S, 610A, 621R, 621R/659G, 636G, 642E, 642E/692D, 659A, 659G, 659N, 659R, 659T, 662D, 662F, 662H, 662Q, 662T, 662V, 662W, 673K, 683K, 684P, 688A, 688C, 688G, 689G, 690G, 690R, 690S, 695K, 725H, 741G, 741S, 744C, 744G/793P, 744L, 744Q, 744R, 744W, 751G, 801S, 801T, 810C, 810D, 810F, 810G, 810N, 818A, 818E, 818F, 818L, 818R, 818S, 830M, 832F, 832L, and 832Y, or combinations thereof, wherein the amino acid positions are relative to the reference sequence of SEQ ID NO: 2994.

In some embodiments, the engineered DNA polymerase comprises one or more substitutions or substitution sets selected from H4A, H4F, H4G, H4R, H4T, H4V, H6F, H6G, H6N, H6R, H6V, S10I, S10Q, S10R, G11E, G11L, G11R, V12D, V12W, P14E, P14E/Q50H, P14E/Q50H/A56V/P598K/T725H, P14E/Q50H/P598K/Y695K, P14E/P95D, P14E/P95D/R501K/Q642E, P14E/R139D/K249E/E433K, P14E/R139D/T725H, P14E/E167D, P14E/E167D/P197K/E433K, P14E/E167D/P197K/R501K/T725H, P14E/E167D/P197K/Q642E/Y695K, P14E/E167D/K249E/E433K/P598K/Q642E, P14E/E167D/K249E/R501K/P598K/Q642E, P14E/E167D/K249E/P598K/Y695K/T725H, P14E/E167D/K249E/Q642E, P14E/E167D/Q642E, P14E/E167D/Y695K, P14E/E167D/T725H, P14E/P197K/K249E/Q642E, P14E/P197K/Q642E, P14E/K249E/E433K/T725H, P14E/K249E/P598K, P14E/K249E/Q642E, P14E/K249E/T725H, P14E/E433K/P598K, P14E/R501K, P14E/P598K, P14E/P598K/Y695K, P14E/P598K/Y695K/T725H, P14E/Q642E/T725H, P18H, P18H/P302V, P18H/P302V/L531V/T673K/A684P, P18H/P302V/L547M, P18H/F484V/T673K, P18H/F484V/T673K/A684P, P18H/S524D/L547M, P18H/L547M, P18H/A684P, H29M, H29N, H29V, H29W, S45L/Q268G/S299N/P302L/S321T/S621R/S659G, S45L/Q268G/P302L/S621R/E721R, S45L/L376F/L385F/T408A/N494V/A525R/S621R/E696Q, S45L/A423E, P48M, P48V, Q50H, Q50H/E433K/P598K, Q50H/R501K, Q50H/P598K, Q50H/P598K/Y695K, Q50H/Q642E, Y53A, Y53Q, Y53W, A76L, A76S, A76W, A76Y, K77H, K77M, K77R, K77S, A91C, A91R, A91S, A91W, P95D/E167D/P197K/P598K/Q642E/Y695K, P95D/E167D/P197K/P598K/Y695K/T725H, P95D/K249E/E433K/Q642E, P95D/K249E/P598K, P95D/P598K, D99G, D99L, D99P, D99S, D99T, A105E, A105F, A105G, A105L, A105S, A105T, P122G, P122I, P122L, P122N, P122R, P122S, R139D/E167D, R139D/E167D/P197K/E433K/R501K/Q642E, R139D/E433K/P598K, G141C, E167D, E167D/P197K, E167D/P197K/Q642E/Y695K/T725H, E167D/Q642E, E167D/T725H, W177C, W177L, W177Q, W177R, P197K/Q642E/T725H, P197K/Y695K, P202G, V204L, E209G, E209M, E209N, E209V, K217G, K217R, K217T, K227M, K227S, E239I, E239L, E239W, E239Y, D247A, D247G, K249E/Y695K, K249E/T725H, D266E, Q268G, Q268G/P302V/A451G/N494S/S621R, Q268H, Q268S, R270P, L291Q, L291Q/A684P, S299E, S299F, S299G, S299K, S299M, S299P, S299R/A474V, S299V, P302V, P302V/A423E/A451G/N494S/S621R, P302V/A423E/S621R, P302V/A451G/N494S, G313F, G313S, G313T, G313V, D346H, D346M, D346P, D346R, D346T, G349N, G349P, G349R, G349T, G349V, G349W, E356A, E356G, E356I, E356Q, E356R, E356V, L361G, L370D, L370T, L370Y, L374G, P377G, P377L, T394C, T394D, T394G, T394M, T394R, G404C, G404K, G404R, G404S, G404V, A423E, A423E/A451G/S621R/E696Q, A423E/N494V/A525R, A423F, A423G, A423R, A423S, A423V, L426A, L426G, L426H, L426Q, L426R/L798F, L426V, L426W, G427A, G427C, G427E, G427K, G427L, G427M, G427R, G427S, G427T, G427W, E433K/R501K/T725H, L436E, L436G, L436H, L436K, L436S, L436W, L436Y, Y439F/L509V/L7891, E440C, E440G, E440W, K444T, K444V, P445G/L446V, P445H, L446M/L811I, R448G, R448K, R448Q, R448S, R448T, R448V/H793P, K459F, K459L, K459R, D461A, E482A, F484V, F484V/A684P, R485C, R485D, R485F, R485M, R485N, R485Q, R485S, R485T, R489S, R489W, N494V, Q498M/A684P, L509A, K514R, K514S, K517I, K517N, K517R, K517S, K517T, A525R/E529D/S621R, E533C, E533R, Q543V/S621R/E721R, L547M, K565C, K565R, K565S, K565T, N574L, N574Q, R596A, R596V, P598K, P598K/Q642E, P598K/Q642E/Y695K, P598K/Y695K, P598K/T725H, Q601S, E610A, S621R, S621R/S659G, N636G, Q642E, Q642E/G692D, S659A, S659G, S659N, S659R, S659T, A662D, A662F, A662H, A662Q, A662T, A662V, A662W, T673K, S683K, A684P, S688A, S688C, S688G, Q689G, E690G, E690R, E690S, Y695K, T725H, D741G, D741S, A744C, A744G/H793P, A744L, A744Q, A744R, A744W, E751G, P801S, P801T, A810C, A810D, A810F, A810G, A810N, G818A, G818E, G818F, G818L, G818R, G818S, V830M, 1832F, 1832L, and I832Y, or combinations thereof, wherein the amino acid positions are relative to the reference sequence of SEQ ID NO: 2994.

In some embodiments, the engineered DNA polymerase comprises one or more substitutions or substitution sets at positions selected from 4, 6, 10, 11, 14, 14/50, 14/139/725, 14/167, 14/167/642, 14/167/695, 14/598, 18/302/531/673/684, 18/524/547, 18/684, 45/268/299/302/321/621/659, 45/268/302/621/721, 48, 50/598, 77, 95/167/197/598/695/725, 99, 105, 122, 167, 167/197, 177, 209, 217, 239, 249/695, 268, 346, 349, 356, 423, 423/451/621/696, 426, 426/798, 427, 433/501/725, 436, 439/509/789, 445, 448, 448/793, 482, 484, 484/684, 514, 517, 533, 543/621/721, 565, 574, 598/695, 621, 621/659, 659, 662, 684, 688, 690, 725, 744/793, 801, 810, and 832, or combinations thereof, wherein the amino acid positions are relative to the reference sequence of SEQ ID NO: 2994.

In some embodiments, the engineered DNA polymerase comprises one or more substitutions or substitution sets selected from 4G, 6G, 6R, 101, 11E, 11R, 14E, 14E/50H, 14E/139D/725H, 14E/167D, 14E/167D/642E, 14E/167D/695K, 14E/598K, 18H/302V/531V/673K/684P, 18H/524D/547M, 18H/684P, 45L/268G/299N/302L/321T/621R/659G, 45L/268G/302L/621R/721R, 48V, 50H/598K, 77M, 95D/167D/197K/598K/695K/725H, 99S, 99T, 105F, 105T, 122G, 122R, 167D, 167D/197K, 177L, 209V, 217G, 2391, 249E/695K, 268S, 346P, 346R, 349P, 349R, 349V, 356V, 423E/451G/621R/696Q, 423G, 423S, 426G, 426H, 426R/798F, 426W, 427A, 427C, 427L, 427R, 433K/501K/725H, 436E, 436K, 436S, 439F/509V/7891, 445H, 448S, 448V/793P, 482A, 484V, 484V/684P, 514R, 514S, 5171, 517T, 533C, 543V/621R/721R, 565R, 574L, 598K/695K, 621R, 621R/659G, 659G, 659R, 662H, 662T, 662W, 684P, 688A, 688C, 688G, 690G, 725H, 744G/793P, 801T, 810D, 810F, 810N, and 832L, or combinations thereof, wherein the amino acid positions are relative to the reference sequence of SEQ ID NO: 2994.

In some embodiments, the engineered DNA polymerase comprises one or more substitutions or substitution sets selected from H4G, H6G, H6R, S10I, G11E, G11R, P14E, P14E/Q50H, P14E/R139D/T725H, P14E/E167D, P14E/E167D/Q642E, P14E/E167D/Y695K, P14E/P598K, P18H/P302V/L531V/T673K/A684P, P18H/S524D/L547M, P18H/A684P, S45L/Q268G/S299N/P302L/S321T/S621R/S659G, S45L/Q268G/P302L/S621R/E721R, P48V, Q50H/P598K, K77M, P95D/E167D/P197K/P598K/Y695K/T725H, D99S, D99T, A105F, A105T, P122G, P122R, E167D, E167D/P197K, W177L, E209V, K217G, E239I, K249E/Y695K, Q268S, D346P, D346R, G349P, G349R, G349V, E356V, A423E/A451G/S621R/E696Q, A423G, A423S, L426G, L426H, L426R/L798F, L426W, G427A, G427C, G427L, G427R, E433K/R501K/T725H, L436E, L436K, L436S, Y439F/L509V/L789I, P445H, R448S, R448V/H793P, E482A, F484V, F484V/A684P, K514R, K514S, K517I, K517T, E533C, Q543V/S621R/E721R, K565R, N574L, P598K/Y695K, S621R, S621R/S659G, S659G, S659R, A662H, A662T, A662W, A684P, S688A, S688C, S688G, E690G, T725H, A744G/H793P, P801T, A810D, A810F, A810N, and I832L, or combinations thereof, wherein the amino acid positions are relative to the reference sequence of SEQ ID NO: 2994.

In some embodiments, the engineered DNA polymerase comprises one or more substitutions or substitution sets at amino acid positions selected from 11, 12, 18/547, 45/268/299/302/321/621/659, 45/268/302/621/721, 45/376/385/408/494/525/621/696, 48, 105, 122, 302, 346, 426/798, 439/509/789, 448/793, 484, 514, 525/529/621, 533, 543/621/721, 574, 690, and 744/793, or combinations thereof, wherein the amino acid positions are relative to the reference sequence of SEQ ID NO: 2994.

In some embodiments, the engineered DNA polymerase comprises one or more substitutions or substitution sets selected from 11E, 12W, 18H/547M, 45L/268G/299N/302L/321T/621R/659G, 45L/268G/302L/621R/721R, 45L/376F/385F/408A/494V/525R/621R/696Q, 48M, 105F, 122G, 302V, 346R, 426R/798F, 439F/509V/7891, 448V/793P, 484V, 514S, 525R/529D/621R, 533C, 543V/621R/721R, 574Q, 690G, and 744G/793P, or combinations thereof, wherein the amino acid positions are relative to the reference sequence of SEQ ID NO: 2994.

In some embodiments, the engineered DNA polymerase comprises one or more substitutions or substitution sets selected from G11E, V12W, P18H/L547M, S45L/Q268G/S299N/P302L/S321T/S621R/S659G, S45L/Q268G/P302L/S621R/E721R, S45L/L376F/L385F/T408A/N494V/A525R/S621R/E696Q, P48M, A105F, P122G, P302V, D346R, L426R/L798F, Y439F/L509V/L789I, R448V/H793P, F484V, K514S, A525R/E529D/S621R, E533C, Q543V/S621R/E721R, N574Q, E690G, and A744G/H793P, or combinations thereof, wherein the amino acid positions are relative to the reference sequence of SEQ ID NO: 2994.

In some embodiments, the engineered DNA polymerase comprises one or more substitutions or substitution sets at positions selected from 11, 12, 18, 18/302, 18/302/547, 18/547, 45/268/299/302/321/621/659, 45/268/302/621/721, 45/376/385/408/494/525/621/696, 48, 105, 122, 291/684, 302, 346, 426/798, 439/509/789, 448/793, 484, 514, 525/529/621, 533, 547, 574, 684, 690, and 744/793, or combinations thereof, wherein the amino acid positions are relative to the reference sequence of SEQ ID NO: 2994.

In some embodiments, the engineered DNA polymerase comprises one or more substitutions or substitution sets selected from 11E, 12W, 18H, 18H/302V, 18H/302V/547M, 18H/547M, 45L/268G/299N/302L/321T/621R/659G, 45L/268G/302L/621R/721R, 45L/376F/385F/408A/494V/525R/621R/696Q, 48M, 105F, 122G, 291Q/684P, 302V, 346R, 426R/798F, 439F/509V/789I, 448V/793P, 484V, 514S, 525R/529D/621R, 533C, 547M, 574Q, 684P, 690G, and 744G/793P, or combinations thereof, wherein the amino acid positions are relative to the reference sequence of SEQ ID NO: 2994.

In some embodiments, the engineered DNA polymerase comprises one or more substitutions or substitution sets selected from G11E, V12W, P18H, P18H/P302V, P18H/P302V/L547M, P18H/L547M, S45L/Q268G/S299N/P302L/S321T/S621R/S659G, S45L/Q268G/P302L/S621R/E721R, S45L/L376F/L385F/T408A/N494V/A525R/S621R/E696Q, P48M, A105F, P122G, L291Q/A684P, P302V, D346R, L426R/L798F, Y439F/L509V/L789I, R448V/H793P, F484V, K514S, A525R/E529D/S621R, E533C, L547M, N574Q, A684P, E690G, and A744G/H793P, or combinations thereof, wherein the amino acid positions are relative to the reference sequence of SEQ ID NO: 2994.

The present invention also provides engineered DNA polymerases or functional fragments thereof, comprising a polypeptide sequence having at least 75% or more sequence identity to a reference sequence comprising residues 12 to 841 of SEQ ID NO: 3796, or to a reference sequence of SEQ ID NO: 3796, wherein the polypeptide sequence comprises one or more substitutions relative to the reference sequence comprising residues 12 to 841 of SEQ ID NO: 3796, or to the reference sequence of SEQ ID NO: 3796.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 3796 or to the reference sequence of SEQ ID NO: 3796.

In some embodiments, the engineered DNA polymerase comprises one or more substitutions or substitution sets at positions selected from 1/2/149, 15/167, 18, 18/105/139/197/249/426/598, 18/105/139/197/291/299/423/426/448/529/547/725, 18/105/249/284/302/423/426/501/673/684/798, 18/105/321/547, 18/139/197/249/299/302/433/547/642/659/695/744, 18/139/197/426/598, 18/139/197/642, 18/139/249/423/433/501/547/725, 18/197/249/291/484, 18/197/321/426/448/451/659/673/744, 18/197/321/433/484, 18/249, 18/249/299/302/423/529/533/642/659/673, 18/249/302/423/484/547/695, 18/249/448/484/673, 18/249/673, 18/291/302/423/684, 18/291/302/433, 18/291/302/525/547/659/684/695, 18/291/302/684, 18/291/423/525/529, 18/291/659, 18/291/695, 18/299/302/423/426/525/529, 18/299/451/525/529/695, 18/302/423/529, 18/302/433/547/684, 18/302/525/529/547, 18/302/529/659/695, 18/321/423, 18/321/484/547/725, 18/423, 18/423/529/695, 18/433, 18/433/673/725, 18/484, 18/598/725, 32, 38/463, 64, 105/139/291/302/423/484/547/725, 105/139/448/501/525/529/642/659, 105/197/249/423/426/547/642/673, 105/321/433/744, 105/321/501, 137, 139/249/291/321/484/695/725, 139/249/291/451/744, 139/249/426/451/501/673/744, 149, 160, 164/172, 167, 172, 197/249/327/423/673/690, 197/302/391/547/598, 197/525/529/659/744, 197/547, 197/684/725, 200/577, 245/423, 247/423/448, 249/321/673/798, 249/423/426/673, 249/448/451/547/659/725, 249/725/744/798, 253, 256, 256/455, 271, 281, 291/302/423, 291/302/529, 291/321/423/725, 291/423/525, 291/423/529/533/598/642/725/798, 291/484/598/642/673/744/798, 291/525, 300, 302, 302/364/376/383/386/408/451/484/598, 302/423/433/529/695, 302/423/525/529, 302/423/547/684/695, 302/684/695, 311, 314, 316, 321/448/529/673, 323, 330, 334/431, 347, 348, 352/363, 353, 354, 358, 361, 363, 368, 372, 376, 376/699, 383, 389, 392, 394, 411, 414, 422, 423/424/547, 423/433/484/529/533/642, 423/433/487/525/547, 423/484/673, 423/529/684, 423/547/598/642, 430, 431, 433/525/529/659/684/695, 436/743, 448/451/529/725, 451/598/725, 455, 455/518, 462, 463, 464, 468, 469, 472, 474, 474/821, 476, 479, 480, 484, 489, 491, 492, 498, 501, 504/548, 505, 510, 519/720, 521, 524, 525, 525/533, 529, 531, 532, 533, 535, 540, 546, 547, 550, 553, 555, 555/572, 557, 558, 559, 566, 568, 570, 571, 572, 573, 574, 575, 576, 577, 583, 588, 590, 590/652, 592, 597, 600, 601, 612, 615, 618, 620, 622, 623, 625, 628, 643, 654, 655, 660, 664, 675, 677, 682, 685, 690, 691/692, 692, 692/766, 697, 703, 705, 708, 711, 715, 720, 726, 728, 730, 732, 738, 743, 746, 752, 753, 756, 766, 769, 770, 779, 793, 800, 802, 803, 805, 813, 819, 825, 827, and 829, or combinations thereof, wherein the amino acid positions are relative to the reference sequence of SEQ ID NO: 3796.

In some embodiments, the engineered DNA polymerase comprises one or more substitutions or substitution sets selected from 1H/2M/149G, 15M/167V, 18H, 18H/105F/139D/197K/249E/426R/598K, 18H/105F/139D/197K/291Q/299N/423E/426R/448V/529D/547M/725H, 18H/105F/249E/284C/302V/423E/426R/501K/673K/684P/798F, 18H/105F/321T/547M, 18H/139D/197K/249E/299N/302V/433K/547M/642E/659G/695K/744G, 18H/139D/197K/426R/598K, 18H/139D/197K/642E, 18H/139D/249E/423E/433K/501K/547M/725H, 18H/197K/249E/291Q/484V, 18H/197K/321T/426R/448V/451G/659G/673K/744G, 18H/197K/321T/433K/484V, 18H/249E, 18H/249E/299N/302V/423E/529D/533C/642E/659G/673K, 18H/249E/302V/423E/484V/547M/695K, 18H/249E/448V/484V/673K, 18H/249E/673K, 18H/291Q/302V/423E/684P, 18H/291Q/302V/433K, 18H/291Q/302V/525R/547M/659G/684P/695K, 18H/291Q/302V/684P, 18H/291Q/423E/525R/529D, 18H/291Q/659G, 18H/291Q/695K, 18H/299N/302V/423E/426R/525R/529D, 18H/299N/451G/525R/529D/695K, 18H/302V/423E/529D, 18H/302V/433K/547M/684P, 18H/302V/525R/529D/547M, 18H/302V/529D/659G/695K, 18H/321T/423E, 18H/321T/484V/547M/725H, 18H/423E, 18H/423E/529D/695K, 18H/433K, 18H/433K/673K/725H, 18H/484V, 18H/598K/725H, 32G, 32L, 32S, 38M/463G, 64G, 105F/139D/291Q/302V/423E/484V/547M/725H, 105F/139D/448V/501K/525R/529D/642E/659G, 105F/197K/249E/423E/426R/547M/642E/673K, 105F/321T/433K/744G, 105F/321T/501K, 137L, 139D/249E/291Q/321T/484V/695K/725H, 139D/249E/291Q/451G/744G, 139D/249E/426R/451G/501K/673K/744G, 149G, 149P, 149V, 160G, 160L, 164V/172F, 167A, 167G, 172H, 172V, 172W, 197K/249E/327L/423E/673K/690G, 197K/302V/391S/547M/598K, 197K/525R/529D/659G/744G, 197K/547M, 197K/684P/725H, 200S/577G, 2451/423L, 247Y/423E/448V, 249E/321T/673K/798F, 249E/423E/426R/673K, 249E/448V/451G/547M/659G/725H, 249E/725H/744G/798F, 253C, 253I, 253M, 256F, 256S, 256S/455F, 256V, 271L, 281V, 291Q/302V/423E, 291Q/302V/529D, 291Q/321T/423E/725H, 291Q/423E/525R, 291Q/423E/529D/533C/598K/642E/725H/798F, 291Q/484V/598K/642E/673K/744G/798F, 291Q/525R, 300G, 302G, 302M, 302S, 302V/364N/376F/383K/386G/408A/451G/484V/598K, 302V/423E/433K/529D/695K, 302V/423E/525R/529D, 302V/423E/547M/684P/695K, 302V/684P/695K, 311C, 311G, 311H, 311K, 314L, 314M, 316N, 321T/448V/529D/673K, 323W, 330M, 330T, 330V, 334T/431C, 347L, 347R, 348A, 352S/363L, 3531, 353T, 354G, 354M, 358E, 358L, 361C, 361R, 361W, 363L, 363T, 363V, 368A, 368L, 368R, 368W, 372K, 372L, 372R, 372S, 372W, 376I, 376K, 376W, 376W/699L, 383I, 383R, 383S, 389G, 389R, 389S, 392G, 392R, 392T, 392V, 394D, 411E, 414S, 422A, 422G, 422L, 422Q, 422R, 423E/424K/547M, 423E/433K/484V/529D/533C/642E, 423E/433K/487V/525R/547M, 423E/484V/673K, 423E/529D/684P, 423E/547M/598K/642E, 430R, 431D, 431Q, 431S, 431T, 431V, 433K/525R/529D/659G/684P/695K, 436F/743K, 448V/451G/529D/725H, 451G/598K/725H, 455E, 455G, 455L, 455P/518I, 455Q, 455S, 455V, 455W, 462A, 462G, 462L, 462Q, 462R, 462S, 462W, 463T, 464H, 464K, 464V, 468A, 468G, 468T, 468W, 469R, 472E, 472G, 472L, 472S, 472T, 474E, 474L/821S, 474R, 474S, 476S, 479W, 480G, 484E, 484G, 484L, 484Q, 484T, 484W, 489H, 489L, 489Q, 491W, 492K, 492Y, 498C, 498D, 501A, 501L, 501S, 504S/548F, 505V, 510G, 510S, 510T, 519D/720S, 521W, 524K, 525G, 525Q, 525R, 525R/533C, 525S, 525V, 529S, 531A, 532L, 533G, 533L, 533N, 533R, 533W, 535N, 535S, 540C, 540S, 540T, 546C, 546R, 547M, 550G, 550I, 550P, 550Y, 553L, 553S, 555A, 555L, 555T/572H, 555V, 557L, 558Q, 559L, 559R, 566G, 568C, 568K, 570S, 571Y, 572N, 572T, 573T, 574M, 575C, 575R, 576K, 576W, 577G, 577H, 577L, 577S, 577W, 583M, 583Q, 588F, 588G, 588V, 590F, 590I/652T, 592S, 597G, 597S, 597V, 600A, 600K, 600R, 600S, 601F, 601L, 612E, 612S, 612T, 612V, 615S, 615Y, 618C, 620L, 620M, 620T, 622G, 622L, 622S, 623G, 623L, 623V, 623W, 6251, 625T, 628M, 628T, 643G, 643L, 643M, 643V, 643Y, 654F, 654G, 654K, 655A, 655F, 655L, 655V, 660E, 660G, 660R, 660W, 664L, 664M, 675G, 675L, 675M, 675T, 677A, 682K, 685R, 685S, 690G, 690R, 690T, 690V, 690W, 691F/692H, 692L, 692S/766T, 692V, 697G, 697Q, 697S, 703F, 703G, 703K, 703P, 703R, 703V, 705F, 705G, 705M, 705V, 708R, 711S, 711V, 711W, 715Q, 715V, 720G, 726T, 728A, 728F, 728Q, 728R, 728S, 728V, 728W, 730R, 730V, 732E, 738G, 738M, 738W, 743A, 743G, 743Q, 743R, 743W, 746-, 752L, 752V, 753G, 753K, 753L, 753R, 756H, 756R, 756T, 766G, 766V, 769A, 769C, 769G, 769M, 769V, 770R, 779R, 793T, 800G, 800I, 800M, 800P, 802C, 802G, 802R, 803S, 805F, 805G, 805I, 805M, 805Q, 805T, 813G, 819A, 819L, 819R, 819S, 819W, 825G, 825S, 825Y, 827G, 827V, 827W, 829L, 829P, and 829W, or combinations thereof, wherein the amino acid positions are relative to the reference sequence of SEQ ID NO: 3796.

In some embodiments, the engineered DNA polymerase comprises one or more substitutions or substitution sets selected from M1H/H2M/A149G, V15M/E167V, P18H, P18H/A105F/R139D/P197K/K249E/L426R/P598K, P18H/A105F/R139D/P197K/L291Q/S299N/A423E/L426R/R448V/E529D/L547M/T725H, P18H/A105F/K249E/R284C/L302V/A423E/L426R/R501K/T673K/A684P/L798F, P18H/A105F/S321T/L547M, P18H/R139D/P197K/K249E/S299N/L302V/E433K/L547M/Q642E/S659G/Y695K/A744G, P18H/R139D/P197K/L426R/P598K, P18H/R139D/P197K/Q642E, P18H/R139D/K249E/A423E/E433K/R501K/L547M/T725H, P18H/P197K/K249E/L291Q/F484V, P18H/P197K/S321T/L426R/R448V/A451G/S659G/T673K/A744G, P18H/P197K/S321T/E433K/F484V, P18H/K249E, P18H/K249E/S299N/L302V/A423E/E529D/E533C/Q642E/S659G/T673K, P18H/K249E/L302V/A423E/F484V/L547M/Y695K, P18H/K249E/R448V/F484V/T673K, P18H/K249E/T673K, P18H/L291Q/L302V/A423E/A684P, P18H/L291Q/L302V/E433K, P18H/L291Q/L302V/A525R/L547M/S659G/A684P/Y695K, P18H/L291Q/L302V/A684P, P18H/L291Q/A423E/A525R/E529D, P18H/L291Q/S659G, P18H/L291Q/Y695K, P18H/S299N/L302V/A423E/L426R/A525R/E529D, P18H/S299N/A451G/A525R/E529D/Y695K, P18H/L302V/A423E/E529D, P18H/L302V/E433K/L547M/A684P, P18H/L302V/A525R/E529D/L547M, P18H/L302V/E529D/S659G/Y695K, P18H/S321T/A423E, P18H/S321T/F484V/L547M/T725H, P18H/A423E, P18H/A423E/E529D/Y695K, P18H/E433K, P18H/E433K/T673K/T725H, P18H/F484V, P18H/P598K/T725H, Y32G, Y32L, Y32S, L38M/A463G, K64G, A105F/R139D/L291Q/L302V/A423E/F484V/L547M/T725H, A105F/R139D/R448V/R501K/A525R/E529D/Q642E/S659G, A105F/P197K/K249E/A423E/L426R/L547M/Q642E/T673K, A105F/S321T/E433K/A744G, A105F/S321T/R501K, A137L, R139D/K249E/L291Q/S321T/F484V/Y695K/T725H, R139D/K249E/L291Q/A451G/A744G, R139D/K249E/L426R/A451G/R501K/T673K/A744G, A149G, A149P, A149V, R160G, R160L, L164V/T172F, E167A, E167G, T172H, T172V, T172W, P197K/K249E/W327L/A423E/T673K/E690G, P197K/L302V/P391S/L547M/P598K, P197K/A525R/E529D/S659G/A744G, P197K/L547M, P197K/A684P/T725H, N200S/A577G, L245I/A423L, D247Y/A423E/R448V, K249E/S321T/T673K/L798F, K249E/A423E/L426R/T673K, K249E/R448V/A451G/L547M/S659G/T725H, K249E/T725H/A744G/L798F, E253C, E253I, E253M, R256F, R256S, R256S/A455F, R256V, R271L, F281V, L291Q/L302V/A423E, L291Q/L302V/E529D, L291Q/S321T/A423E/T725H, L291Q/A423E/A525R, L291Q/A423E/E529D/E533C/P598K/Q642E/T725H/L798F, L291Q/F484V/P598K/Q642E/T673K/A744G/L798F, L291Q/A525R, P300G, L302G, L302M, L302S, L302V/D364N/L376F/M383K/A386G/T408A/A451G/F484V/P598K, L302V/A423E/E433K/E529D/Y695K, L302V/A423E/A525R/E529D, L302V/A423E/L547M/A684P/Y695K, L302V/A684P/Y695K, P311C, P311G, P311H, P311K, A314L, A314M, L316N, S321T/R448V/E529D/T673K, P323W, L330M, L330T, L330V, A334T/G431C, P347L, P347R, H348A, R352S/K363L, G353I, G353T, L354G, L354M, R358E, R358L, L361C, L361R, L361W, K363L, K363T, K363V, V368A, V368L, V368R, V368W, E372K, E372L, E372R, E372S, E372W, L376I, L376K, L376W, L376W/Q699L, M383I, M383R, M383S, L389G, L389R, L389S, S392G, S392R, S392T, S392V, T394D, A411E, R414S, H422A, H422G, H422L, H422Q, H422R, A423E/N424K/L547M, A423E/E433K/F484V/E529D/E533C/Q642E, A423E/E433K/A487V/A525R/L547M, A423E/F484V/T673K, A423E/E529D/A684P, A423E/L547M/P598K/Q642E, Q430R, G431D, G431Q, G431S, G431T, G431V, E433K/A525R/E529D/S659G/A684P/Y695K, L436F/N743K, R448V/A451G/E529D/T725H, A451G/P598K/T725H, A455E, A455G, A455L, A455P/T518I, A455Q, A455S, A455V, A455W, V462A, V462G, V462L, V462Q, V462R, V462S, V462W, A463T, Y464H, Y464K, Y464V, L468A, L468G, L468T, L468W, S469R, V472E, V472G, V472L, V472S, V472T, A474E, A474L/P821S, A474R, A474S, M476S, L479W, E480G, F484E, F484G, F484L, F484Q, F484T, F484W, R489H, R489L, R489Q, F491W, R492K, R492Y, Q498C, Q498D, R501A, R501L, R501S, F504S/A548F, D505V, P510G, P510S, P510T, G519D/L720S, R521W, S524K, A525G, A525Q, A525R, A525R/E533C, A525S, A525V, E529S, L531A, R532L, E533G, E533L, E533N, E533R, E533W, H535N, H535S, K540C, K540S, K540T, E546C, E546R, L547M, L550G, L550I, L550P, L550Y, T553L, T553S, 1555A, 1555L, 1555T/R572H, 1555V, R557L, L558Q, P559L, P559R, T566G, R568C, R568K, H570S, T571Y, R572N, R572T, F573T, N574M, Q575C, Q575R, T576K, T576W, A577G, A577H, A577L, A577S, A577W, L583M, L583Q, P588F, P588G, P588V, L590F, L590I/A652T, N592S, T597G, T597S, T597V, G600A, G600K, G600R, G600S, Q601F, Q601L, G612E, G612S, G612T, G612V, L615S, L615Y, L618C, Y620L, Y620M, Y620T, Q622G, Q622L, Q622S, 1623G, I623L, 1623V, 1623W, L625I, L625T, L628M, L628T, E643G, E643L, E643M, E643V, E643Y, W654F, W654G, W654K, M655A, M655F, M655L, M655V, A660E, A660G, A660R, A660W, D664L, D664M, N675G, N675L, N675M, N675T, G677A, M682K, F685R, F685S, E690G, E690R, E690T, E690V, E690W, L691F/G692H, G692L, G692S/A766T, G692V, E697G, E697Q, E697S, Q703F, Q703G, Q703K, Q703P, Q703R, Q703V, Y705F, Y705G, Y705M, Y705V, S708R, K711S, K711V, K711W, W715Q, W715V, L720G, R726T, Y728A, Y728F, Y728Q, Y728R, Y728S, Y728V, Y728W, E730R, E730V, L732E, Y738G, Y738M, Y738W, N743A, N743G, N743Q, N743R, N743W, V746-, A752L, A752V, A753G, A753K, A753L, A753R, M756H, M756R, M756T, A766G, A766V, L769A, L769C, L769G, L769M, L769V, M770R, P779R, H793T, A800G, A800I, A800M, A800P, K802C, K802G, K802R, E803S, A805F, A805G, A805I, A805M, A805Q, A805T, K813G, V819A, V819L, V819R, V819S, V819W, P825G, P825S, P825Y, E827G, E827V, E827W, E829L, E829P, and E829W, or combinations thereof, wherein the amino acid positions are relative to the reference sequence of SEQ ID NO: 3796.

The present invention also provides engineered DNA polymerases or functional fragments thereof, comprising a polypeptide sequence having at least 75% or more sequence identity to a reference sequence comprising residues 12 to 841 of SEQ ID NO: 4182, or to a reference sequence of SEQ ID NO: 4182, wherein the polypeptide sequence comprises one or more substitutions relative to the reference sequence comprising residues 12 to 841 of SEQ ID NO: 4182, or to the reference sequence of SEQ ID NO: 4182.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 4182 or to the reference sequence of SEQ ID NO: 4182.

In some embodiments, the engineered DNA polymerase comprises one or more substitutions or substitution sets at positions selected from 18/149/300/423/424/743/769, 18/256/423/424/455/673, 18/372/424/533/546/600/673/769, 18/424, 149/256/300/302/424/484/769, 149/256/300/372/424/546, 149/424, 256/300/363/423/424/546/673/690, 424, 424/533, and 424/723, or combinations thereof, wherein the amino acid positions are relative to the reference sequence of SEQ ID NO: 4182.

In some embodiments, the engineered DNA polymerase comprises one or more substitutions or substitution sets selected from 18H/149G/300G/423A/424N/743A/769C, 18H/256S/423A/424N/455S/673K, 18H/372S/424N/533W/546C/600A/673K/769C, 18H/424N, 149G/256S/300G/302G/424N/484V/769C, 149G/256S/300G/372S/424N/546C, 149G/424N, 256S/300G/363L/423A/424N/546C/673K/690V, 424N, 424N/533W, and 424N/723D, or combinations thereof, wherein the amino acid positions are relative to the reference sequence of SEQ ID NO: 4182.

In some embodiments, the engineered DNA polymerase comprises one or more substitutions or substitution sets selected from P18H/A149G/P300G/E423A/K424N/N743A/L769C, P18H/R256S/E423A/K424N/A455S/T673K, P18H/E372S/K424N/E533W/E546C/G600A/T673K/L769C, P18H/K424N, A149G/R256S/P300G/L302G/K424N/F484V/L769C, A149G/R256S/P300G/E372S/K424N/E546C, A149G/K424N, R256S/P300G/K363L/E423A/K424N/E546C/T673K/E690V, K424N, K424N/E533W, and K424N/G723D, or combinations thereof, wherein the amino acid positions are relative to the reference sequence of SEQ ID NO: 4182.

The present invention also provides engineered DNA polymerases or functional fragments thereof, comprising a polypeptide sequence having at least 75% or more sequence identity to a reference sequence comprising residues 12 to 841 of SEQ ID NO: 5094, or to a reference sequence of SEQ ID NO: 5094, wherein the polypeptide sequence comprises one or more substitutions relative to the reference sequence comprising residues 12 to 841 of SEQ ID NO: 5094, or to the reference sequence of SEQ ID NO: 5094.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5094 or to the reference sequence of SEQ ID NO: 5094.

In some embodiments, the engineered DNA polymerase comprises one or more substitutions or substitution sets at positions selected from 9/689, 9/691, 21/695/700, 45, 45/268, 45/268/302/423, 45/268/302/467/621/748/749, 45/268/302/469, 45/268/302/469/547/557/721/749, 45/268/302/475/489, 45/268/302/547/557/621/748/749, 45/268/423/467/748, 45/268/423/469/721, 45/268/423/475/547/721/749, 45/268/469, 45/268/475, 45/268/475/489, 45/268/475/621, 45/268/489/621/749, 45/268/621/721, 45/302, 45/302/423/469/475, 45/302/423/469/621, 45/302/423/469/621/721/748/749, 45/302/423/547/621/721, 45/302/469/547/721, 45/302/475/621/748/749, 45/302/621, 45/423, 45/423/467, 45/423/467/469/547/721/749, 45/423/467/469/749, 45/423/467/475/547/621/721/749, 45/423/467/621, 45/423/469/475, 45/423/469/475/621/721/749, 45/423/469/489/621, 45/423/475/547/721, 45/423/475/557/621/749, 45/423/489, 45/423/489/557, 45/467/469/557, 45/467/621, 45/467/748, 45/469/721, 45/469/749, 45/475/489/547/749, 45/547/749, 45/557, 45/721, 83/700/744, 95/268/302/423/489/547/557/721/748/749, 116, 228/708, 268, 268/302/423/557/721, 268/302/721, 268/423, 268/423/467/547, 268/423/469, 268/423/469/475, 268/467, 268/467/469/475, 268/467/748, 268/475/547, 268/475/557/721/749, 268/489/547, 296/433, 302, 302/423/467/621/748, 302/423/469/489/721/748/749, 302/423/475/489/547/621, 302/423/547/721, 302/423/557, 302/467/469/547/557, 302/467/489/748/749, 302/467/621, 302/467/721/748, 302/469, 302/469/489/621/721/748, 302/489, 302/489/547/621/699/749, 302/557/721, 302/621, 302/721, 302/749, 306, 306/459, 314/538, 321, 322, 329/359/611/700/744, 332, 335, 339, 341/532, 344, 345, 351, 354/359/408/598, 359, 359/408/412, 359/408/412/611/614/744, 359/408/412/611/708/744, 359/408/412/611/744/789, 359/408/412/700/744, 359/408/412/744, 359/408/433/614/708, 359/408/598/700, 359/408/611/614/744, 359/408/695/700/744, 359/408/695/744, 359/412, 359/412/433/436/598/611/614, 359/412/433/611/614/708, 359/412/433/611/614/744/789, 359/412/436/598/611/695/744, 359/412/469/611/695/708/744, 359/412/598, 359/412/598/611/695/700/708/744, 359/412/598/611/744, 359/412/695/700/744, 359/412/695/708/744, 359/433/611/614/700, 359/433/611/695/700/789, 359/436/611/708/744, 359/469/611, 359/469/611/614, 359/469/611/700, 359/598/611/614/744, 359/598/614/695/708/744, 359/598/700/744, 359/598/744/789, 359/611/614/695/700/744, 359/611/614/744, 359/611/700/744, 359/614, 359/695/700/789, 359/700, 359/744, 359/789, 364, 373, 378, 385, 386, 387/630, 393, 397, 399, 404, 408, 408/412, 408/412/433/436/611/614/744, 408/412/433/598/611/700/708/744, 408/412/436/598/708, 408/412/436/611/614, 408/412/469/475/611/695/708/744, 408/412/598/611/614/708, 408/412/598/700/744, 408/412/611/695/708, 408/412/708/744, 408/433/700/708/744, 408/436/598/611/708/744, 408/436/695/708/744, 408/598/611, 408/598/611/614/700, 408/598/695/700, 408/611/614/695/708, 408/611/614/708/744, 408/611/614/744, 408/611/744, 408/700, 408/744, 409, 412, 412/433/598/611/744, 412/433/611/744, 412/436/611/614/744/789, 412/436/744, 412/469/611/695/744, 412/469/611/700/744, 412/598/611/744, 412/611/614, 412/611/614/708/744/789, 412/611/789, 412/614/708, 412/689, 412/695/789, 412/708/744, 413, 417, 423, 423/467/469, 423/467/489/621/721, 423/467/547/557/621/699/721, 423/467/621/721/749, 423/467/721, 423/469, 423/469/475/489/721/748/749, 423/469/489/557/621/721/749, 423/469/547/557/621/749, 423/469/547/557/749, 423/469/621, 423/469/721, 423/489, 423/489/621, 423/598/700/708/744, 423/721, 423/748, 424, 425, 426, 433, 433/436/598/700/744, 433/436/598/744, 433/436/611/614, 433/469/611, 433/611/695/700/744, 433/744/789, 436, 436/611/614, 436/611/614/695, 436/700/744, 436/708/744, 439, 443, 446, 448, 449, 467/469, 467/489/547/621/748, 467/547/721, 467/621, 467/721, 467/721/748, 467/748, 467/749, 469, 469/471, 469/473, 469/475/621/721/748, 469/475/749, 469/614/695, 469/614/695/744, 469/621, 469/621/748, 469/744, 469/748, 475, 483, 485/791, 487, 489, 506, 507, 509, 511, 514, 517, 520, 532, 533/542, 538, 539, 539/571, 542, 546/699, 547, 547/621/721/748/749, 549, 554, 557, 557/621, 580, 584, 587, 596, 598, 598/611, 598/611/614, 598/611/708, 598/614/700/744, 598/708, 602, 602/724, 611, 611/614, 611/614/708/744/789, 611/744, 614, 614/700/708/744/789, 614/744, 614/789, 617, 621, 621/639/748/749, 621/745, 621/748, 621/749, 630, 652, 689, 691, 695, 695/708/744, 695/744, 699, 700, 700/744, 700/767, 708, 714, 721, 721/749, 723, 725, 741, 744, 745, 748, 748/749, 767, 777, 780, 785, 785/809, 787, 789, 791, 795, 798, 799, 811, 817, 821, 836, 838, and 839, or combinations thereof, wherein the amino acid positions are relative to the reference sequence of SEQ ID NO: 5094.

In some embodiments, the engineered DNA polymerase comprises one or more substitutions or substitution sets selected from 9D/691S, 9V/689G, 21S/695L/700G, 45R, 45R/268R, 45R/268R/302P/423A, 45R/268R/302P/467A/621S/748S/749V, 45R/268R/302P/469H, 45R/268R/302P/469H/547L/557P/721E/749V, 45R/268R/302P/475E/489H, 45R/268R/302P/547L/557P/621S/748S/749V, 45R/268R/423A/467A/748S, 45R/268R/423A/469H/721E, 45R/268R/423A/475E/547L/721E/749V, 45R/268R/469H, 45R/268R/475E, 45R/268R/475E/489H, 45R/268R/475E/621S, 45R/268R/489H/621S/749V, 45R/268R/621S/721E, 45R/302P, 45R/302P/423A/469H/475E, 45R/302P/423A/469H/621S, 45R/302P/423A/469H/621S/721E/748S/749V, 45R/302P/423A/547L/621S/721E, 45R/302P/469H/547L/721E, 45R/302P/475E/621S/748S/749V, 45R/302P/621S, 45R/423A, 45R/423A/467A, 45R/423A/467A/469H/547L/721E/749V, 45R/423A/467A/469H/749V, 45R/423A/467A/475E/547L/621S/721E/749V, 45R/423A/467A/621S, 45R/423A/469H/475E, 45R/423A/469H/475E/621S/721E/749V, 45R/423A/469H/489H/621S, 45R/423A/475E/547L/721E, 45R/423A/475E/557P/621S/749V, 45R/423A/489H, 45R/423A/489H/557P, 45R/467A/469H/557P, 45R/467A/621S, 45R/467A/748S, 45R/469H/721E, 45R/469H/749V, 45R/475E/489H/547L/749V, 95S/268R/302P/423A/489H/547L/557P/721E/748S/749V, 116P, 228D/708E, 268R, 268R/302P/423A/557P/721E, 268R/302P/721E, 268R/423A, 268R/423A/467A/547L, 268R/423A/469H, 268R/423A/469H/475E, 268R/467A, 268R/467A/469H/475E, 268R/467A/748S, 268R/475E/547L, 268R/475E/557P/721E/749V, 268R/489H/547L, 296H/433L, 302P, 302P/423A/467A/621S/748S, 302P/423A/469H/489H/721E/748S/749V, 302P/423A/475E/489H/547L/621S, 302P/423A/547L/721E, 302P/423A/557P, 302P/467A/469H/547L/557P, 302P/467A/489H/748S/749V, 302P/467A/621S, 302P/467A/721E/748S, 302P/469H, 302P/469H/489H/621S/721E/748S, 302P/489H, 302P/489H/547L/621S/699A/749V, 302P/557P/721E, 302P/621S, 302P/721E, 302P/749V, 306E, 306L, 306M, 306N, 306P, 306R, 306S, 306T, 306T/459E, 306V, 314S/538L, 321A, 321I, 321W, 322A, 322C, 322D, 322G, 322L, 322M, 322P, 322S, 322T, 329K/359T/611P/700G/744S, 332C, 332D, 332I, 332L, 332M, 332Q, 332R, 332T, 332V, 335C, 335G, 335S, 339A, 339H, 339L, 339P, 339S, 339V, 341M/532L, 344G, 344P, 344R, 344S, 344V, 345G, 345K, 345L, 351M, 351V, 354I/359T/408W/598F, 359T, 359T/408W/412G, 359T/408W/412G/611K/614A/744S, 359T/408W/412G/611K/744S/789D, 359T/408W/412G/611P/708G/744S, 359T/408W/412G/700G/744G, 359T/408W/412G/744S, 359T/408W/433A/614A/708G, 359T/408W/598F/700G, 359T/408W/611K/614A/744G, 359T/408W/695L/700G/744G, 359T/408W/695L/744G, 359T/412G, 359T/412G/433A/436R/598F/611P/614A, 359T/412G/433A/611P/614A/708G, 359T/412G/433A/611P/614A/744S/789D, 359T/412G/436R/598F/611P/695L/744G, 359T/412G/469L/611P/695L/708G/744S, 359T/412G/598F, 359T/412G/598F/611K/744S, 359T/412G/598F/611P/695L/700G/708G/744S, 359T/412G/695L/700G/744G, 359T/412G/695L/708G/744G, 359T/433A/611P/614A/700G, 359T/433A/611P/695L/700G/789D, 359T/436R/611P/708G/744G, 359T/469L/611K, 359T/469L/611P/614A, 359T/469L/611P/700G, 359T/598F/611P/614A/744S, 359T/598F/614A/695L/708G/744S, 359T/598F/700G/744G, 359T/598F/744S/789D, 359T/611K/614A/744G, 359T/611P/614A/695L/700G/744G, 359T/611P/700G/744S, 359T/614A, 359T/695L/700G/789D, 359T/700G, 359T/744S, 359T/789D, 364G, 364P, 364R, 364T, 364V, 373F, 373K, 373P, 373S, 373V, 378C, 378F, 378G, 378L, 378R, 378Y, 385A, 385E, 385G, 385P, 385R, 385V, 386C, 386G, 386L, 386Q, 386S, 386V, 386W, 387C/630E, 393A, 393E, 393G, 393P, 393R, 393Y, 397A, 397D, 397G, 397L, 397N, 397P, 397R, 397V, 399E, 399G, 399L, 399P, 399Q, 399R, 399S, 399W, 404F, 404I, 404K, 404L, 404P, 404S, 404T, 404W, 408A, 408L, 408N, 408P, 408Q, 408R, 408W, 408W/412G, 408W/412G/433A/436R/611P/614A/744G, 408W/412G/433A/598F/611P/700G/708G/744S, 408W/412G/436R/598F/708G, 408W/412G/436R/611P/614A, 408W/412G/469L/475Q/611P/695L/708G/744S, 408W/412G/598F/611P/614A/708G, 408W/412G/598F/700G/744S, 408W/412G/611P/695L/708G, 408W/412G/708G/744G, 408W/433A/700G/708G/744S, 408W/436R/598F/611P/708G/744S, 408W/598F/695L/700G, 408W/611K/614A/744G, 408W/611P/614A/695L/708G, 408W/611P/614A/708G/744G, 408W/611P/744S, 408W/700G, 408W/744G, 408W/744S, 409G, 412F, 412G, 412G/433A/598F/611P/744G, 412G/433A/611P/744S, 412G/436R/611K/614A/744S/789D, 412G/436R/744G, 412G/469L/611P/695L/744G, 412G/469L/611P/700G/744S, 412G/598F/611P/744S, 412G/611K/614A, 412G/611P/614A/708G/744S/789D, 412G/611P/789D, 412G/614A/708G, 412G/695L/789D, 412G/708G/744G, 412K, 412L, 412P, 412Q, 412R, 412T/689V, 413E, 413G, 413H, 413L, 413R, 413S, 413T, 413W, 417A, 417G, 417Q, 417S, 423A, 423A/467A/469H, 423A/467A/489H/621S/721E, 423A/467A/547L/557P/621S/699A/721E, 423A/467A/621S/721E/749V, 423A/467A/721E, 423A/469H, 423A/469H/475E/489H/721E/748S/749V, 423A/469H/489H/557P/621S/721E/749V, 423A/469H/547L/557P/621S/749V, 423A/469H/547L/557P/749V, 423A/469H/621S, 423A/469H/721E, 423A/489H, 423A/489H/621S, 423A/721E, 423A/748S, 423G/598F/700G/708G/744S, 424E, 424G, 424L, 424T, 425C, 425S, 425T, 425Y, 426G, 426R, 426S, 426T, 426V, 433A, 433A/436R/598F/700G/744G, 433A/436R/598F/744G, 433A/436R/611P/614A, 433A/469L/611P, 433A/611P/695L/700G/744G, 433A/744S/789D, 433G, 433Q, 433R, 433S, 433W, 436A, 436G, 436Q, 436R, 436R/611K/614A, 436R/611P/614A/695L, 436R/700G/744G, 436R/708G/744G, 436S, 436T, 436V, 436W, 439A, 439E, 439L, 439S, 439W, 443C, 443T, 446S, 448E, 448L, 448W, 449G, 449W, 467A/469H, 467A/489H/547L/621S/748S, 467A/547L/721E, 467A/621S, 467A/721E, 467A/721E/748S, 467A/748S, 467A/749V, 467C/469H, 467H/469H, 467M/469H, 467R/469H, 467T/469H, 467V/469H, 467W/469H, 469E, 469H, 469H/471A, 469H/471F, 469H/471H, 469H/471L, 469H/471M, 469H/471Q, 469H/471R, 469H/473F, 469H/473W, 469H/475E/621S/721E/748S, 469H/475E/749V, 469H/621S, 469H/621S/748S, 469H/748S, 469L, 469L/614A/695L, 469L/614A/695L/744S, 469L/744S, 469R, 469W, 469Y, 475E, 483A, 483C, 483G, 483L, 483Q, 483S, 483T, 485H/791S, 487G, 487L, 487R, 487S, 487V, 487Y, 489H, 506G, 506H, 506M, 506R, 506V, 507G, 507K, 507Q, 509F, 509M, 509V, 511A, 511D, 511L, 511N, 511R, 511V, 511Y, 514G, 514M, 514T, 514W, 517A, 517L, 517M, 517R, 517S, 517V, 517W, 520S, 520V, 532T, 532W, 532Y, 533K/542S, 538A, 538G, 538L, 538P, 538S, 539A, 539G, 539G/571A, 539L, 542A, 542G, 542K, 542P, 542R, 542S, 542W, 546G/699A, 547L, 547L/621S/721E/748S/749V, 549A, 549H, 549L, 549M, 549N, 549P, 549S, 549T, 549V, 549Y, 554S, 557P, 557P/621S, 580K, 584A, 584Q, 584V, 587G, 587M, 587S, 587T, 587V, 587W, 596W, 598A, 598C, 598F, 598F/611P, 598F/611P/614A, 598F/611P/708G, 598F/614A/700G/744G, 598F/708G, 598G, 598L, 598R, 598S, 598V, 602A, 602G, 602S, 602S/724H, 602V, 602W, 611C, 611I, 611K, 611L, 611P, 611P/614A, 611P/614A/708G/744S/789D, 611P/744S, 611R, 611S, 611T, 611V, 611W, 614A, 614A/700G/708G/744S/789D, 614A/744G, 614A/789D, 614G, 614P, 614T, 614W, 617C, 617D, 617G, 617H, 617L, 617R, 617S, 617V, 621H/745D, 621S, 630W, 652G, 652S, 652V, 689G, 689K, 689L, 689M, 689P, 689R, 689S, 689W, 691A, 691G, 691K, 691Q, 691R, 691S, 691T, 691W, 695A, 695E, 695L, 695L/708G/744G, 695L/744G, 695P, 695R, 695S, 695T, 699A, 700E, 700F, 700G, 700G/744G, 700G/767T, 700L, 700R, 700V, 700W, 700Y, 708A, 708G, 708R, 708T, 714G, 714L, 714P, 714R, 714S, 714V, 714Y, 721E, 721E/749V, 723A, 723Q, 723T, 723V, 725C, 725E, 725L, 725P, 725S, 725V, 725W, 741A, 741E, 741H, 741K, 741L, 741P, 744F, 744G, 744H, 744P, 744R, 744S, 744T, 744W, 745C, 745D, 745G, 745P, 745Q, 745V, 745W, 748D, 748R, 748S, 748S/749V, 748V, 767G, 767T, 777A, 777C, 777Q, 777T, 777V, 780A, 780R, 785Q, 785R, 785R/809V, 787E, 787G, 787K, 787P, 787W, 789A, 789D, 789E, 789G, 789P, 789Q, 789S, 789T, 789V, 789Y, 791A, 791G, 791H, 791L, 791R, 791T, 791V, 795D, 795S, 798T, 799M, 799Q, 811G, 811R, 811S, 811W, 817A, 817D, 817F, 817H, 817K, 817N, 817Q, 817S, 817V, 817W, 821D, 821G, 821K, 821L, 821Q, 821S, 836L, 838R, 838T, 838V, 839E, 839L, 839P, 839R, 839T, and 839V, or combinations thereof, wherein the amino acid positions are relative to the reference sequence of SEQ ID NO: 5094.

In some embodiments, the engineered DNA polymerase comprises one or more substitutions or substitution sets selected from G9D/L691S, G9V/Q689G, R21S/Y695L/A700G, L45R, L45R/G268R, L45R/G268R/L302P/E423A, L45R/G268R/L302P/N467A/R621S/G748S/K749V, L45R/G268R/L302P/S469H, L45R/G268R/L302P/S469H/M547L/R557P/R721E/K749V, L45R/G268R/L302P/H475E/R489H, L45R/G268R/L302P/M547L/R557P/R621S/G748S/K749V, L45R/G268R/E423A/N467A/G748S, L45R/G268R/E423A/S469H/R721E, L45R/G268R/E423A/H475E/M547L/R721E/K749V, L45R/G268R/S469H, L45R/G268R/H475E, L45R/G268R/H475E/R489H, L45R/G268R/H475E/R621S, L45R/G268R/R489H/R621S/K749V, L45R/G268R/R621S/R721E, L45R/L302P, L45R/L302P/E423A/S469H/H475E, L45R/L302P/E423A/S469H/R621S, L45R/L302P/E423A/S469H/R621S/R721E/G748S/K749V, L45R/L302P/E423A/M547L/R621S/R721E, L45R/L302P/S469H/M547L/R721E, L45R/L302P/H475E/R621S/G748S/K749V, L45R/L302P/R621S, L45R/E423A, L45R/E423A/N467A, L45R/E423A/N467A/S469H/M547L/R721E/K749V, L45R/E423A/N467A/S469H/K749V, L45R/E423A/N467A/H475E/M547L/R621S/R721E/K749V, L45R/E423A/N467A/R621S, L45R/E423A/S469H/H475E, L45R/E423A/S469H/H475E/R621S/R721E/K749V, L45R/E423A/S469H/R489H/R621S, L45R/E423A/H475E/M547L/R721E, L45R/E423A/H475E/R557P/R621S/K749V, L45R/E423A/R489H, L45R/E423A/R489H/R557P, L45R/N467A/S469H/R557P, L45R/N467A/R621S, L45R/N467A/G748S, L45R/S469H/R721E, L45R/S469H/K749V, L45R/H475E/R489H/M547L/K749V, L45R/M547L/K749V, L45R/R557P, L45R/R721E, H83N/A700G/A744G, P95S/G268R/L302P/E423A/R489H/M547L/R557P/R721E/G748S/K749V, L116P, N228D/S708E, G268R, G268R/L302P/E423A/R557P/R721E, G268R/L302P/R721E, G268R/E423A, G268R/E423A/N467A/M547L, G268R/E423A/S469H, G268R/E423A/S469H/H475E, G268R/N467A, G268R/N467A/S469H/H475E, G268R/N467A/G748S, G268R/H475E/M547L, G268R/H475E/R557P/R721E/K749V, G268R/R489H/M547L, L296H/E433L, L302P, L302P/E423A/N467A/R621S/G748S, L302P/E423A/S469H/R489H/R721E/G748S/K749V, L302P/E423A/H475E/R489H/M547L/R621S, L302P/E423A/M547L/R721E, L302P/E423A/R557P, L302P/N467A/S469H/M547L/R557P, L302P/N467A/R489H/G748S/K749V, L302P/N467A/R621S, L302P/N467A/R721E/G748S, L302P/S469H, L302P/S469H/R489H/R621S/R721E/G748S, L302P/R489H, L302P/R489H/M547L/R621S/Q699A/K749V, L302P/R557P/R721E, L302P/R621S, L302P/R721E, L302P/K749V, A306E, A306L, A306M, A306N, A306P, A306R, A306S, A306T, A306T/K459E, A306V, A314S/V538L, S321A, S321I, S321W, R322A, R322C, R322D, R322G, R322L, R322M, R322P, R322S, R322T, E329K/G359T/E611P/A700G/A744S, A332C, A332D, A332I, A332L, A332M, A332Q, A332R, A332T, A332V, A335C, A335G, A335S, G339A, G339H, G339L, G339P, G339S, G339V, V341M/R532L, A344G, A344P, A344R, A344S, A344V, E345G, E345K, E345L, L351M, L351V, L354I/G359T/T408W/P598F, G359T, G359T/T408W/A412G, G359T/T408W/A412G/E611K/R614A/A744S, G359T/T408W/A412G/E611K/A744S/L789D, G359T/T408W/A412G/E611P/S708G/A744S, G359T/T408W/A412G/A700G/A744G, G359T/T408W/A412G/A744S, G359T/T408W/E433A/R614A/S708G, G359T/T408W/P598F/A700G, G359T/T408W/E611K/R614A/A744G, G359T/T408W/Y695L/A700G/A744G, G359T/T408W/Y695L/A744G, G359T/A412G, G359T/A412G/E433A/L436R/P598F/E611P/R614A, G359T/A412G/E433A/E611P/R614A/S708G, G359T/A412G/E433A/E611P/R614A/A744S/L789D, G359T/A412G/L436R/P598F/E611P/Y695L/A744G, G359T/A412G/S469L/E611P/Y695L/S708G/A744S, G359T/A412G/P598F, G359T/A412G/P598F/E611K/A744S, G359T/A412G/P598F/E611P/Y695L/A700G/S708G/A744S, G359T/A412G/Y695L/A700G/A744G, G359T/A412G/Y695L/S708G/A744G, G359T/E433A/E611P/R614A/A700G, G359T/E433A/E611P/Y695L/A700G/L789D, G359T/L436R/E611P/S708G/A744G, G359T/S469L/E611K, G359T/S469L/E611P/R614A, G359T/S469L/E611P/A700G, G359T/P598F/E611P/R614A/A744S, G359T/P598F/R614A/Y695L/S708G/A744S, G359T/P598F/A700G/A744G, G359T/P598F/A744S/L789D, G359T/E611K/R614A/A744G, G359T/E611P/R614A/Y695L/A700G/A744G, G359T/E611P/A700G/A744S, G359T/R614A, G359T/Y695L/A700G/L789D, G359T/A700G, G359T/A744S, G359T/L789D, D364G, D364P, D364R, D364T, D364V, G373F, G373K, G373P, G373S, G373V, P378C, P378F, P378G, P378L, P378R, P378Y, L385A, L385E, L385G, L385P, L385R, L385V, A386C, A386G, A386L, A386Q, A386S, A386V, A386W, Y387C/H630E, N393A, N393E, N393G, N393P, N393R, N393Y, E397A, E397D, E397G, E397L, E397N, E397P, E397R, E397V, V399E, V399G, V399L, V399P, V399Q, V399R, V399S, V399W, G404F, G404I, G404K, G404L, G404P, G404S, G404T, G404W, T408A, T408L, T408N, T408P, T408Q, T408R, T408W, T408W/A412G, T408W/A412G/E433A/L436R/E611P/R614A/A744G, T408W/A412G/E433A/P598F/E611P/A700G/S708G/A744S, T408W/A412G/L436R/P598F/S708G, T408W/A412G/L436R/E611P/R614A, T408W/A412G/S469L/H475Q/E611P/Y695L/S708G/A744S, T408W/A412G/P598F/E611P/R614A/S708G, T408W/A412G/P598F/A700G/A744S, T408W/A412G/E611P/Y695L/S708G, T408W/A412G/S708G/A744G, T408W/E433A/A700G/S708G/A744S, T408W/L436R/P598F/E611P/S708G/A744S, T408W/L436R/Y695L/S708G/A744G, T408W/P598F/E611P, T408W/P598F/E611P/R614A/A700G, T408W/P598F/Y695L/A700G, T408W/E611K/R614A/A744G, T408W/E611P/R614A/Y695L/S708G, T408W/E611P/R614A/S708G/A744G, T408W/E611P/A744S, T408W/A700G, T408W/A744G, T408W/A744S, E409G, A412F, A412G, A412G/E433A/P598F/E611P/A744G, A412G/E433A/E611P/A744S, A412G/L436R/E611K/R614A/A744S/L789D, A412G/L436R/A744G, A412G/S469L/E611P/Y695L/A744G, A412G/S469L/E611P/A700G/A744S, A412G/P598F/E611P/A744S, A412G/E611K/R614A, A412G/E611P/R614A/S708G/A744S/L789D, A412G/E611P/L789D, A412G/R614A/S708G, A412G/Y695L/L789D, A412G/S708G/A744G, A412K, A412L, A412P, A412Q, A412R, A412T/Q689V, K413E, K413G, K413H, K413L, K413R, K413S, K413T, K413W, L417A, L417G, L417Q, L417S, E423A, E423A/N467A/S469H, E423A/N467A/R489H/R621S/R721E, E423A/N467A/M547L/R557P/R621S/Q699A/R721E, E423A/N467A/R621S/R721E/K749V, E423A/N467A/R721E, E423A/S469H, E423A/S469H/H475E/R489H/R721E/G748S/K749V, E423A/S469H/R489H/R557P/R621S/R721E/K749V, E423A/S469H/M547L/R557P/R621S/K749V, E423A/S469H/M547L/R557P/K749V, E423A/S469H/R621S, E423A/S469H/R721E, E423A/R489H, E423A/R489H/R621S, E423A/R721E, E423A/G748S, E423G/P598F/A700G/S708G/A744S, N424E, N424G, N424L, N424T, L425C, L425S, L425T, L425Y, L426G, L426R, L426S, L426T, L426V, E433A, E433A/L436R/P598F/A700G/A744G, E433A/L436R/P598F/A744G, E433A/L436R/E611P/R614A, E433A/S469L/E611P, E433A/E611P/Y695L/A700G/A744G, E433A/A744S/L789D, E433G, E433Q, E433R, E433S, E433W, L436A, L436G, L436Q, L436R, L436R/E611K/R614A, L436R/E611P/R614A/Y695L, L436R/A700G/A744G, L436R/S708G/A744G, L436S, L436T, L436V, L436W, Y439A, Y439E, Y439L, Y439S, Y439W, E443C, E443T, L446S, R448E, R448L, R448W, V449G, V449W, N467A/S469H, N467A/R489H/M547L/R621S/G748S, N467A/M547L/R721E, N467A/R621S, N467A/R721E, N467A/R721E/G748S, N467A/G748S, N467A/K749V, N467C/S469H, N467H/S469H, N467M/S469H, N467R/S469H, N467T/S469H, N467V/S469H, N467W/S469H, S469E, S469H, S469H/E471A, S469H/E471F, S469H/E471H, S469H/E471L, S469H/E471M, S469H/E471Q, S469H/E471R, S469H/A473F, S469H/A473W, S469H/H475E/R621S/R721E/G748S, S469H/H475E/K749V, S469H/R621S, S469H/R621S/G748S, S469H/G748S, S469L, S469L/R614A/Y695L, S469L/R614A/Y695L/A744S, S469L/A744S, S469R, S469W, S469Y, H475E, V483A, V483C, V483G, V483L, V483Q, V483S, V483T, R485H/Q791S, A487G, A487L, A487R, A487S, A487V, A487Y, R489H, E506G, E506H, E506M, E506R, E506V, L507G, L507K, L507Q, L509F, L509M, L509V, P511A, P511D, P511L, P511N, P511R, P511V, P511Y, K514G, K514M, K514T, K514W, K517A, K517L, K517M, K517R, K517S, K517V, K517W, K520S, K520V, R532T, R532W, R532Y, E533K/L542S, V538A, V538G, V538L, V538P, V538S, D539A, D539G, D539G/T571A, D539L, L542A, L542G, L542K, L542P, L542R, L542S, L542W, E546G/Q699A, M547L, M547L/R621S/R721E/G748S/K749V, K549A, K549H, K549L, K549M, K549N, K549P, K549S, K549T, K549V, K549Y, Y554S, R557P, R557P/R621S, T580K, S584A, S584Q, S584V, D587G, D587M, D587S, D587T, D587V, D587W, R596W, P598A, P598C, P598F, P598F/E611P, P598F/E611P/R614A, P598F/E611P/S708G, P598F/R614A/A700G/A744G, P598F/S708G, P598G, P598L, P598R, P598S, P598V, K602A, K602G, K602S, K602S/R724H, K602V, K602W, E611C, E611I, E611K, E611L, E611P, E611P/R614A, E611P/R614A/S708G/A744S/L789D, E611P/A744S, E611R, E611S, E611T, E611V, E611W, R614A, R614A/A700G/S708G/A744S/L789D, R614A/A744G, R614A/L789D, R614G, R614P, R614T, R614W, A617C, A617D, A617G, A617H, A617L, A617R, A617S, A617V, R621H/R745D, R621S, R621S/R639H/G748S/K749V, R621S/G748S, R621S/K749V, H630A, H630C, H630F, H630G, H630M, H630S, H630T, H630W, A652G, A652S, A652V, Q689G, Q689K, Q689L, Q689M, Q689P, Q689R, Q689S, Q689W, L691A, L691G, L691K, L691Q, L691R, L691S, L691T, L691W, Y695A, Y695E, Y695L, Y695L/S708G/A744G, Y695L/A744G, Y695P, Y695R, Y695S, Y695T, Q699A, A700E, A700F, A700G, A700G/A744G, A700G/A767T, A700L, A700R, A700V, A700W, A700Y, S708A, S708G, S708R, S708T, A714G, A714L, A714P, A714R, A714S, A714V, A714Y, R721E, R721E/K749V, G723A, G723Q, G723T, G723V, T725C, T725E, T725L, T725P, T725S, T725V, T725W, D741A, D741E, D741H, D741K, D741L, D741P, A744F, A744G, A744H, A744P, A744R, A744S, A744T, A744W, R745C, R745D, R745G, R745P, R745Q, R745V, R745W, G748D, G748R, G748S, G748S/K749V, G748V, A767G, A767T, L777A, L777C, L777Q, L777T, L777V, Q780A, Q780R, G785Q, G785R, G785R/A809V, R787E, R787G, R787K, R787P, R787W, L789A, L789D, L789E, L789G, L789P, L789Q, L789S, L789T, L789V, L789Y, Q791A, Q791G, Q791H, Q791L, Q791R, Q791T, Q791V, E795D, E795S, L798T, E799M, E799Q, L811G, L811R, L811S, L811W, E817A, E817D, E817F, E817H, E817K, E817N, E817Q, E817S, E817V, E817W, P821D, P821G, P821K, P821L, P821Q, P821S, W836L, A838R, A838T, A838V, A839E, A839L, A839P, A839R, A839T, and A839V, or combinations thereof, wherein the amino acid positions are relative to the reference sequence of SEQ ID NO: 5094.

In some embodiments, the engineered DNA polymerase comprises one or more substitutions or substitution sets at positions selected from 45/268/302/469, 45/268/475, 45/268/475/489, 45/268/621/721, 45/423/467/469/749, 45/721, 268, 268/475/547, 302, 302/489/547/621/699/749, 302/721, 302/749, 306, 359, 359/412/433/611/614/744/789, 359/436/611/708/744, 385, 408, 412, 412/598/611/744, 417, 423/467/721, 426, 433, 436, 436/708/744, 467/469, 469, 469/475/621/721/748, 469/475/749, 469/621/748, 469/748, 489, 511, 517, 547, 549, 557, 598, 598/611/708, 611, 614, 621/639/748/749, 630, 695, 699, 700, 708, 714, 721, 725, 741, 744, 748, 787, 789, 791, 795, and 817, or combinations thereof, wherein the amino acid positions are relative to the reference sequence of SEQ ID NO: 5094.

In some embodiments, the engineered DNA polymerase comprises one or more substitutions or substitution sets selected from 45R/268R/302P/469H, 45R/268R/475E, 45R/268R/475E/489H, 45R/268R/621S/721E, 45R/423A/467A/469H/749V, 45R/721E, 268R, 268R/475E/547L, 302P, 302P/489H/547L/621S/699A/749V, 302P/721E, 302P/749V, 306P, 359T, 359T/412G/433A/611P/614A/744S/789D, 359T/436R/611P/708G/744G, 385V, 408W, 412G, 412G/598F/611P/744S, 412P, 417G, 423A/467A/721E, 426R, 433A, 436R, 436R/708G/744G, 467A/469H, 469H/475E/621S/721E/748S, 469H/475E/749V, 469H/621S/748S, 469H/748S, 469L, 489H, 511R, 517R, 547L, 549L, 557P, 598F, 598F/611P/708G, 611K, 611P, 614A, 621S/639H/748S/749V, 630M, 695L, 695P, 699A, 700G, 700R, 708G, 714S, 721E, 725P, 741H, 741L, 744G, 744S, 748S, 787W, 789D, 791R, 795S, 817A, and 817K, or combinations thereof, wherein the amino acid positions are relative to the reference sequence of SEQ ID NO: 5094.

In some embodiments, the engineered DNA polymerase comprises one or more substitutions or substitution sets selected from L45R/G268R/L302P/S469H, L45R/G268R/H475E, L45R/G268R/H475E/R489H, L45R/G268R/R621S/R721E, L45R/E423A/N467A/S469H/K749V, L45R/R721E, G268R, G268R/H475E/M547L, L302P, L302P/R489H/M547L/R621S/Q699A/K749V, L302P/R721E, L302P/K749V, A306P, G359T, G359T/A412G/E433A/E611P/R614A/A744S/L789D, G359T/L436R/E611P/S708G/A744G, L385V, T408W, A412G, A412G/P598F/E611P/A744S, A412P, L417G, E423A/N467A/R721E, L426R, E433A, L436R, L436R/S708G/A744G, N467A/S469H, S469H/H475E/R621S/R721E/G748S, S469H/H475E/K749V, S469H/R621S/G748S, S469H/G748S, S469L, R489H, P511R, K517R, M547L, K549L, R557P, P598F, P598F/E611P/S708G, E611K, E611P, R614A, R621S/R639H/G748S/K749V, H630M, Y695L, Y695P, Q699A, A700G, A700R, S708G, A714S, R721E, T725P, D741H, D741L, A744G, A744S, G748S, R787W, L789D, Q791R, E795S, E817A, and E817K, or combinations thereof, wherein the amino acid positions are relative to the reference sequence of SEQ ID NO: 5094.

The present invention also provides engineered DNA polymerases or functional fragments thereof, comprising a polypeptide sequence having at least 75% or more sequence identity to a reference sequence comprising residues 12 to 841 of SEQ ID NO: 5136, or to a reference sequence of SEQ ID NO: 5136, wherein the polypeptide sequence comprises one or more substitutions relative to the reference sequence comprising residues 12 to 841 of SEQ ID NO: 5136, or to the reference sequence of SEQ ID NO: 5136.

In some embodiments, the engineered DNA polymerase comprises a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5136 or to the reference sequence of SEQ ID NO: 5136.

In some embodiments, the engineered DNA polymerase comprises one or more substitutions or substitution sets at positions selected from 359/408/614, 359/412/436/598/611/614/695/700/708/744, 359/412/598/611/614/700/708/744, 359/412/611/708/744, 359/433/744, 359/436/598, 359/469/700/744, 359/598/611, 359/598/611/695/708, 359/598/695, 359/611/700/708, 359/744, 359/789, 408, 408/412/436/614/700/744, 408/412/469, 408/412/611, 408/412/744, 408/433/436/611/614/744, 408/433/436/695/708/744, 408/611, 412, 412/433, 412/611/614/708/744/789, 412/700/744, 433/436/598/611/614/708/744, 433/436/611/614/744, 433/436/611/744, 433/436/744, 433/614/700/744, 433/708, 436/598, 436/598/614/695/744, 436/611, 436/611/614, 436/611/614/695/700/744, 436/611/708, 436/744, 598/611/614/708/744, 598/614/695/700/708/744, 611, 611/614, 611/614/695/700/708/744, 611/708/744, 614/744, 614/789, 695, 695/700/744, 700, 700/744, and 744/789, or combinations thereof, wherein the amino acid positions are relative to the reference sequence of SEQ ID NO: 5136.

In some embodiments, the engineered DNA polymerase comprises one or more substitutions or substitution sets selected from 359T/408W/614R, 359T/412G/436R/598F/611K/614R/695L/700G/708G/744S, 359T/412G/598F/611P/614R/700G/708G/744S, 359T/412G/611K/708G/744G, 359T/433A/744G, 359T/436R/598F, 359T/469L/700G/744G, 359T/598F/611K, 359T/598F/611K/695L/708G, 359T/598F/695L, 359T/611K/700G/708G, 359T/744S, 359T/789D, 408W, 408W/412G/436R/614R/700G/744S, 408W/412G/469L, 408W/412G/611P, 408W/412G/744G, 408W/433A/436R/611P/614R/744G, 408W/433A/436R/695L/708G/744S, 408W/611P, 412G, 412G/433A, 412G/611K/614R/708G/744S/789D, 412G/700G/744S, 433A/436R/598F/611P/614R/708G/744S, 433A/436R/611P/614R/744S, 433A/436R/611P/744G, 433A/436R/744G, 433A/614R/700G/744S, 433A/708G, 436R/598F, 436R/598F/614R/695L/744G, 436R/611P, 436R/611P/614R, 436R/611P/614R/695L/700G/744S, 436R/611P/708G, 436R/744S, 598F/611P/614R/708G/744G, 598F/614R/695L/700G/708G/744S, 611P, 611P/614R, 611P/614R/695L/700G/708G/744S, 611P/708G/744S, 614R/744G, 614R/789D, 695L, 695L/700G/744G, 700G, 700G/744S, and 744S/789D, or combinations thereof, wherein the amino acid positions are relative to the reference sequence of SEQ ID NO: 5136.

In some embodiments, the engineered DNA polymerase comprises one or more substitutions or substitution sets selected from G359T/T408W/A614R, G359T/A412G/L436R/P598F/E611K/A614R/Y695L/A700G/S708G/A744S, G359T/A412G/P598F/E611P/A614R/A700G/S708G/A744S, G359T/A412G/E611K/S708G/A744G, G359T/E433A/A744G, G359T/L436R/P598F, G359T/S469L/A700G/A744G, G359T/P598F/E611K, G359T/P598F/E611K/Y695L/S708G, G359T/P598F/Y695L, G359T/E611K/A700G/S708G, G359T/A744S, G359T/L789D, T408W, T408W/A412G/L436R/A614R/A700G/A744S, T408W/A412G/S469L, T408W/A412G/E611P, T408W/A412G/A744G, T408W/E433A/L436R/E611P/A614R/A744G, T408W/E433A/L436R/Y695L/S708G/A744S, T408W/E611P, A412G, A412G/E433A, A412G/E611K/A614R/S708G/A744S/L789D, A412G/A700G/A744S, E433A/L436R/P598F/E611P/A614R/S708G/A744S, E433A/L436R/E611P/A614R/A744S, E433A/L436R/E611P/A744G, E433A/L436R/A744G, E433A/A614R/A700G/A744S, E433A/S708G, L436R/P598F, L436R/P598F/A614R/Y695L/A744G, L436R/E611P, L436R/E611P/A614R, L436R/E611P/A614R/Y695L/A700G/A744S, L436R/E611P/S708G, L436R/A744S, P598F/E611P/A614R/S708G/A744G, P598F/A614R/Y695L/A700G/S708G/A744S, E611P, E611P/A614R, E611P/A614R/Y695L/A700G/S708G/A744S, E611P/S708G/A744S, A614R/A744G, A614R/L789D, Y695L, Y695L/A700G/A744G, A700G, A700G/A744S, and A744S/L789D, or combinations thereof, wherein the amino acid positions are relative to the reference sequence of SEQ ID NO: 5136.

In some embodiments, the engineered DNA polymerase comprises one or more substitutions or substitution sets at positions selected from 359/598/695, 408/412/436/614/700/744, 408/611, 412, 433/614/700/744, 436/598, 436/611, 436/611/614/695/700/744, and 700, or combinations thereof, wherein the amino acid positions are relative to the reference sequence of SEQ ID NO: 5136.

In some embodiments, the engineered DNA polymerase comprises one or more substitutions or substitution sets selected from 359T/598F/695L, 408W/412G/436R/614R/700G/744S, 408W/611P, 412G, 433A/614R/700G/744S, 436R/598F, 436R/611P, 436R/611P/614R/695L/700G/744S, and 700G, or combinations thereof, wherein the amino acid positions are relative to the reference sequence of SEQ ID NO: 5136.

In some embodiments, the engineered DNA polymerase comprises one or more substitutions or substitution sets selected from G359T/P598F/Y695L, T408W/A412G/L436R/A614R/A700G/A744S, T408W/E611P, A412G, E433A/A614R/A700G/A744S, L436R/P598F, L436R/E611P, L436R/E611P/A614R/Y695L/A700G/A744S, and A700G, or combinations thereof, wherein the amino acid positions are relative to the reference sequence of SEQ ID NO: 5136.

The present invention further provides engineered DNA polymerases comprising polypeptide sequences having at least 75%, 80%, 85%, 86%, 87%, 88%, 89%, 90%, 91%, 92%, 93%, 94%, 95%, 96%, 97%, 98%, 99%, or more sequence identity to a reference sequence comprising residues 12 to 841 of SEQ ID NO: 178, 994, 1858, 2176, 2310, 2994, 3796, 4182, 5094, and/or 5136, or to a reference sequence of SEQ ID NO: 178, 994, 1858, 2176, 2310, 2994, 3796, 4182, 5094, and/or 5136.

In some embodiments, the engineered DNA polymerase comprises a polypeptide sequence having at least 75%, 80%, 85%, 86%, 87%, 88%, 89%, 90%, 91%, 92%, 93%, 94%, 95%, 96%, 97%, 98%, 99%, or more sequence identity to a reference sequence comprising residues 12 to 841 of SEQ ID NO: 2, 178, 994, 1858, 2176, 2310, 2994, 3796, 4182, 5094, and/or 5136, or to a reference sequence of SEQ ID NO: 2, 178, 994, 1858, 2176, 2310, 2994, 3796, 4182, 5094, and/or 5136, with the proviso that the polypeptide sequence does not include the sequence comprising residues 12 to 841 of SEQ ID NO:2.

In some embodiments, the engineered DNA polymerase comprises a polypeptide sequence comprising one or more amino acid substitutions relative to the reference sequence comprising residues 12 to 841 of SEQ ID NO: 178, or to the reference sequence of SEQ ID NO: 178, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 994, or to the reference sequence of SEQ ID NO: 994, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 1858, or to the reference sequence of SEQ ID NO: 1858, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 2176, or to the reference sequence of SEQ ID NO: 2176, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 2310, or to the reference sequence of SEQ ID NO: 2310, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 2994, or to the reference sequence of SEQ ID NO: 2994, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 3796, or to the reference sequence of SEQ ID NO: 3796, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 4182, or to the reference sequence of SEQ ID NO: 4182, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 5094, or to the reference sequence of SEQ ID NO: 5094, with the proviso that the polypeptide sequence does not include the sequence comprising residues 12 to 841 of SEQ ID NO: 2.

In some embodiments, the engineered DNA polymerase comprises a polypeptide sequence comprising one or more amino acid substitutions relative to the reference sequence comprising residues 12 to 841 of SEQ ID NO: 5136, or to the reference sequence of SEQ ID NO: 5136, with the proviso that the polypeptide sequence does not include the sequence comprising residues 12 to 841 of SEQ ID NO: 2.

In some embodiments, the engineered DNA polymerase comprises a polypeptide sequence comprising a substitution or substitution set provided in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1, wherein the substitution or substitution set is relative to the reference sequence of SEQ ID NO: 2, 178, 994, 1858, 2176, 2310, 2994, 3796, 4182, 5094, and/or 5136.

In some embodiments, the engineered DNA polymerase comprises a polypeptide sequence having at least 75%, 80%, 85%, 86%, 87%, 88%, 89%, 90%, 91%, 92%, 93%, 94%, 95%, 96%, 97%, 98%, 99% or more sequence identity to a sequence comprising residues 12 to 841 of an engineered DNA polymerase variant set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1.

In some embodiments, the engineered DNA polymerase comprises a polypeptide sequence having at least 75%, 80%, 85%, 86%, 87%, 88%, 89%, 90%, 91%, 92%, 93%, 94%, 95%, 96%, 97%, 98%, 99% or more sequence identity to a sequence of an engineered DNA polymerase variant set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1. In some embodiments, the engineered DNA polymerase comprises a polypeptide sequence comprising at least one substitution provided in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1, wherein the substitution is relative to the reference sequence comprising residues 12 to 841 of SEQ ID NO: 178, 994, 1858, 2176, 2310, 2994, 3796, 4182, 5094, and/or 5136, or to the reference sequence of SEQ ID NO: 2, 178, 994, 1858, 2176, 2310, 2994, 3796, 4182, 5094, and/or 5136. In some embodiments, an engineered DNA polymerase comprises a polypeptide sequence comprising at least 75%, 80%, 85%, 86%, 87%, 88%, 89%, 90%, 91%, 92%, 93%, 94%, 95%, 96%, 97%, 98%, 99% or more sequence identity to a reference sequence comprising residues 12 to 841 of an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1 or to a reference sequence of an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1. In some embodiments, the engineered DNA polymerase comprises a polypeptide sequence comprising residues 12 to 841 of an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1, or a polypeptide sequence comprising an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1.

In some embodiments, the engineered DNA polymerase comprises a polypeptide sequence comprising residues 12 to 841 of a sequence selected from the even numbered sequences of SEQ ID NOS: 4-3258 and 3268-6646.

In some embodiments, the engineered DNA polymerase comprises a polypeptide sequence comprising residues 12 to 841 of a sequence selected from the even numbered sequences of SEQ ID NOS: 4-3258 and 3268-6646, wherein the polypeptide optionally has 1, 2, 3, 4, 5, 6, 7, 8, 9, or up to 10 substitutions in the polypeptide sequence.

In some embodiments, the engineered DNA polymerase comprises a polypeptide comprising 1, 2, 3, 4, up to 5 substitutions in the polypeptide sequence.

In some embodiments, the engineered DNA polymerase comprises substitutions that are conservative substitutions. In some embodiments, the engineered DNA polymerase polypeptide has 1, 2, 3, 4, or up to 5 substitutions in the polypeptide sequence. In some embodiments, the engineered DNA polymerase polypeptide has 1, 2, 3, or 4 substitutions in the polypeptide sequence. In some embodiments, the substitutions comprise non-conservative substitutions. In some embodiments, the substitutions comprise conservative substitutions. In some embodiments, the substitutions comprise non-conservative of conservative substitutions. In some embodiments, guidance on non-conservative and conservative substitutions are provided by the variants disclosed herein.

In some embodiments, an engineered DNA polymerase comprises a polypeptide sequence comprising at least 75%, 80%, 85%, 86%, 87%, 88%, 89%, 90%, 91%, 92%, 93%, 94%, 95%, 96%, 97%, 98%, 99% or more sequence identity to a reference sequence comprising residues 12 to 841 of a sequence selected from SEQ ID NOS: 178, 994, 1858, 2176, 2310, 2994, 3796, 4182, 5094, and/or 5136.

In some embodiments, the engineered DNA polymerase comprises a polypeptide sequence comprising a sequence comprising residues 12 to 841 of SEQ ID NO: 178, 994, 1858, 2176, 2310, 2994, 3796, 4182, 5094, and/or 5136.

In some embodiments, the engineered DNA polymerase comprises a polypeptide sequence comprising a sequence of SEQ ID NO: 178, 994, 1858, 2176, 2310, 2994, 3796, 4182, 5094, and/or 5136.

In some embodiments, the foregoing substitutions can be in addition to any of the above described embodiments.

In some embodiments, the engineered DNA polymerase comprises a fusion protein.

It will be apparent that the description herein, including the Examples, provide Tables showing sequence structural information correlating specific amino acid sequence features with the functional activity of the engineered DNA polymerase polypeptides. This structure-function correlation information is provided in the form of specific amino acid residue differences relative to the reference polypeptides of SEQ ID NO: 2, 178, 994, 1858, 2176, 2310, 2994, 3796, 4182, 5094, and/or 5136, as well as associated experimentally determined activity data for the exemplary engineered DNA polymerase polypeptides. Such information provides guidance and information on substitutions implemented in preparing engineered DNA polymerase variants.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 2 and/or compared to the DNA polymerase of SEQ ID NO: 2. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 178 and/or compared to the DNA polymerase of SEQ ID NO: 178. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 994 and/or compared to the DNA polymerase of SEQ ID NO: 994. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 1858 and/or compared to the DNA polymerase of SEQ ID NO: 1858. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 2176 and/or compared to the DNA polymerase of SEQ ID NO: 2176. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 2310 and/or compared to the DNA polymerase of SEQ ID NO: 2310. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 2994 and/or compared to the DNA polymerase of SEQ ID NO: 2994. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 3796 and/or compared to the DNA polymerase of SEQ ID NO: 3796. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 4182 and/or compared to the DNA polymerase of SEQ ID NO: 4182. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 5094 and/or compared to the DNA polymerase of SEQ ID NO: 5094. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase of the present invention has DNA polymerase activity. In some embodiments, the engineered DNA polymerase has at least one improved property as compared to the DNA polymerase comprising residues 12 to 841 of SEQ ID NO: 5136 and/or compared to the DNA polymerase of SEQ ID NO: 5136. In some embodiments, the engineered DNA polymerase has one or more of improved property selected from increased DNA product yield, increased thermostability, increased processivity, increased fidelity, increased DNA template sensitivity, increased tolerance to inhibitors, and/or increased product yield in a PCR reaction.

In some embodiments, the engineered DNA polymerase polypeptides of the present invention are produced by cultivating a host cell, such as a microorganism, comprising at least one polynucleotide sequence encoding at least one engineered DNA polymerase polypeptide under conditions which are conducive for producing the DNA polymerase polypeptide. In some embodiments, the engineered DNA polymerase polypeptide is subsequently recovered from the resulting culture medium and/or cells.

In some embodiments, the engineered DNA polymerase polypeptide described herein is an isolated composition. In some embodiments, the engineered DNA polymerase polypeptide is a purified composition, as further discussed herein.

In some embodiments, the present invention further provides functional fragments or biologically active fragments of engineered DNA polymerase polypeptides described herein. Thus, for each and every embodiment herein of an engineered DNA polymerase, a functional fragment or biologically active fragment of the engineered DNA polymerase is provided herewith. In some embodiments, a functional fragment or biologically active fragments of an engineered DNA polymerase comprises at least about 90%, at least about 95%, at least about 96%, at least about 97%, at least about 98%, or at least about 99% of the activity of the DNA polymerase polypeptide from which it was derived (i.e., the parent DNA polymerase). In some embodiments, functional fragments or biologically active fragments comprise at least about 90%, at least about 91%, at least about 92%, at least about 93%, at least about 94%, at least about 95%, at least about 96%, at least about 97%, at least about 98%, or at least about 99% of the parent sequence of the DNA polymerase. In some embodiments the functional fragment will be truncated by less than 5, less than 10, less than 15, less than 10, less than 25, less than 30, less than 35, less than 40, less than 45, and less than 50 amino acids.

In some embodiments, a functional fragment of an engineered DNA polymerase herein comprises at least 90%, 91%, 92%, 93%, 94%, 95%, 96%, 97%, 98%, or 99% of the parent sequence of the engineered DNA polymerase. In some embodiments the functional fragment will be truncated by less than 5, less than 10, less than 15, less than 10, less than 25, less than 30, less than 35, less than 40, less than 45, less than 50, less than 55, less than 60, less than 65, or less than 70 amino acids.

### Polynucleotides Encoding Engineered Polypeptides, Expression Vectors and Host Cells

In another aspect, the present invention provides polynucleotides encoding the engineered DNA polymerase polypeptides described herein. In some embodiments, the polynucleotides are operatively linked to one or more heterologous regulatory sequences that control gene expression to create a DNA polymerase capable of expressing the polypeptide. In some embodiments, expression constructs containing at least one heterologous polynucleotide encoding the engineered DNA polymerase polypeptide(s) is introduced into appropriate host cells to express the corresponding DNA polymerase polypeptide(s).

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered DNA polymerase polypeptide of the present invention. Thus, the present invention provides methods and compositions for the production of each and every possible variation of engineered DNA polymerase polynucleotides that could be made that encode the engineered DNA polymerase polypeptides described herein by selecting combinations based on the possible codon choices, and all such variations are to be considered specifically disclosed for any polypeptide described herein, including the amino acid sequences presented in the Examples (e.g., in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1) and in the Sequence Listing.

In some embodiments, the codons are preferably optimized for utilization by the chosen host cell for protein production. For example, preferred codons used in bacteria are typically used for expression in bacteria, and preferred codons used in mammalian cells are typically used for expression in mammalian cells. Consequently, codon optimized polynucleotides encoding the engineered DNA polymerase polypeptides contain preferred codons at about 40%, 50%, 60%, 70%, 80%, 90%, or greater than 90% of the codon positions in the full length coding region.

In some embodiments, the codons are preferably optimized for utilization by the chosen host cell for protein production. For example, preferred codons used in bacteria are typically used for expression in bacteria. Consequently, codon optimized polynucleotides encoding the engineered DNA polymerase polypeptides contain preferred codons at about 40%, 50%, 60%, 70%, 80%, 90%, or greater than 90% of the codon positions in the full length coding region.

In some embodiments, the polynucleotide encodes an engineered DNA polymerase polypeptide having DNA polymerase activity, wherein the polypeptide comprises an amino acid sequence having at least at least 70%, at least 75%, or at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a reference sequence comprising residues 12 to 841 of SEQ ID NO: 2, or to a reference sequence of SEQ ID NO: 2, wherein the polypeptide sequence comprises one or more substitutions relative to the reference sequence of SEQ ID NO:2, as described herein.

In some embodiments, the polynucleotide encodes an engineered DNA polymerase polypeptide having DNA polymerase activity, wherein the polypeptide comprises an amino acid sequence having at least at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a reference sequence comprising residues 12 to 841 of SEQ ID NO: 2, or to a reference sequence of SEQ ID NO: 2, wherein the polypeptide sequence comprises one or more substitutions relative to the reference sequence of SEQ ID NO:2, as described herein.

In some embodiments, the present invention provides an engineered polynucleotide encoding an engineered DNA polymerase comprising at least one substitutions or substitution sets selected from 11/287, 15/633, 18/145/255, 20/602, 29, 50/255/557/685, 110, 113/170/194/235/249/319/645/659/660/674/699, 130, 145, 145/255/287/391/472/476/557, 145/255/287/391/476/513, 145/255/287/391/557/685/743/758, 145/255/391/398/472/476/513/557/602/660, 145/255/391/398/472/557/685, 145/255/391/398/476/557/758, 145/255/391/398/513/658/685/743, 145/255/391/472/476/685/743, 145/255/391/472/557/685, 145/255/391/476/557/602/660/743, 145/255/391/476/557/658/660/743, 145/255/391/476/758, 145/255/391/513/557/743/758, 145/255/391/557/758, 145/255/398/472/476/513/557/658/660, 145/255/472/476/557/685/743/758, 145/255/472/513/557/602/660/743, 145/255/513, 145/255/513/602, 145/255/557, 145/255/557/658/660/685, 145/255/743/758, 145/267/391/398/513/557/658/685/743, 145/287/391/472/557/602/658/660/685, 145/287/391/472/602/658, 145/287/391/476/513/557/602, 145/287/391/476/658/660/685, 145/287/658/758, 145/391, 145/391/398/476/758, 145/391/472/476/513/557/658/685/758, 145/391/472/476/557/602/660/685/743, 145/391/472/476/557/743/758, 145/391/472/476/658/660/758, 145/391/472/476/658/685/758, 145/391/472/476/758, 145/391/513/557/602, 145/391/513/557/658/685, 145/391/513/658/743/758, 145/391/513/758, 145/391/557, 145/391/557/658/758, 145/391/557/660/685, 145/391/557/743, 145/398/472/557/660, 145/476/557/660/685/743/758, 145/513/685, 145/557/658, 145/557/685/758, 145/685, 161/162, 161/162/170/194/235/423/699, 161/162/170/235/249/252, 161/162/194/269/605/660, 161/162/269/476/660, 161/162/352/434/605, 161/162/659/660/674, 161/170/235/249/252/434, 161/170/235/269/319, 161/170/235/319/412, 161/170/252/352/412/434/699, 161/170/476, 161/194/198/235/252/423/434/605/645/699, 161/194/235/249/352/412/434/674/699, 161/194/252, 161/194/252/323/352/412/605/659/660/674/699, 161/194/252/412/605, 161/194/434, 161/235/249/319/412/605/659, 161/235/252/319/352/645/674/699, 161/235/352/423/645/674, 161/235/645/699, 161/249/412/476/605/645/659/660/674, 161/252/319/352/412/423/476/605/645, 161/252/319/674, 161/269/434/699, 161/319/660, 161/352, 161/423/645, 161/605, 162, 162/170/194/235/249/269/674/699, 162/170/412/434/476/605, 162/194/249/645/659, 162/235/269/423/476/605/645/659, 162/252/352/412/434/645/660/699, 162/269/352/412/476/605, 162/352, 162/423/645, 162/605/645, 164, 168, 170, 170/194/198/235/674/699, 170/194/235/252/269/323/412/423/476/605/645/660/674, 170/194/235/319/412/476/699, 170/194/269/319/412/423/476/605/645, 170/194/319/352/476/699, 170/235/249/352/645, 170/235/269/352, 170/235/269/434/674, 170/235/319/352/412/434/476/645/660/674, 170/235/352/674/699, 170/235/352/699, 170/235/412, 170/235/645, 170/235/660, 170/249/252/269/352/412/423/434/476/659, 170/249/252/269/423/659, 170/249/412, 170/252/352/423/674/699, 170/269/352/423/476/605/659/660/699, 170/269/476/645, 170/412/423/476/605/645/660/674, 170/476/674, 175, 177, 194, 194/198/235/252/323/412/605/645, 194/198/252/319/423/645/674, 194/235/252, 194/235/252/319/423, 194/235/605, 194/235/645, 194/235/699, 194/249/252/645/699, 194/249/412/476/605/645/699, 194/252/269/319/434/605/645/659/674/699, 194/252/319/412/605, 194/269/319/412/605/674/699, 194/319/352/605/660, 194/323/699, 194/352/476/659, 194/352/605/645/699, 194/434, 194/645/659, 197, 198/674/699, 202/268, 217, 227, 235, 235/249/252/323/352/476, 235/249/252/412/605/645/699, 235/249/252/423/699, 235/249/252/434/605/645/699, 235/252, 235/252/269/352/434/605/674/699, 235/252/269/352/476/645/674, 235/252/269/423/605/645/660/674, 235/269/323/412/434/659/674/699, 235/269/674/699, 235/319/605/645/674, 235/352, 235/352/412/659/660/674/699, 235/352/605, 235/412/645, 235/476/674, 235/605/645, 249/252/319/352/645, 249/252/319/605/660, 249/252/412/423/645/674/699, 249/412/476/605, 252/352/645/660, 252/645, 253, 255/260/398/557, 255/287, 255/287/391/472/557/658/660, 255/287/391/472/658/660/685, 255/287/391/476/557, 255/287/391/658, 255/391, 255/391/398/476/557, 255/391/398/476/602/658/660/685, 255/391/398/513/557/658/743/758, 255/391/472/513/758, 255/391/472/557/658/660/685/743/758, 255/391/472/557/658/660/758, 255/391/472/658/660/758, 255/391/472/685, 255/391/476/513/557/602/685/758, 255/391/476/557/602/660/758, 255/391/476/557/658, 255/391/476/758, 255/391/513/602/658/660, 255/391/557, 255/391/602, 255/391/685/758, 255/391/743, 255/398/472/476/557/658, 255/398/476, 255/398/476/557/658/660/685, 255/398/513/758, 255/472/758, 255/513/557, 255/513/557/685/758, 255/557/658, 255/602, 255/660, 267, 268, 269/352, 269/352/412, 269/605, 270, 271/472, 279, 281, 287/391/472, 287/391/472/476/557/602/658/660/685/743, 287/391/476/513, 287/391/513/557/660/685/743/758, 287/391/557, 287/391/557/602/658/743, 287/391/557/685, 287/391/602, 287/685, 297, 319, 319/352/412/434/699, 319/434/476, 322, 329, 331, 339, 347, 350, 352, 352/412/605/645/699, 352/605/645/699, 352/605/659, 352/645, 354, 356, 358, 360, 361, 365, 372, 377, 379, 391, 391/398/476/685, 391/398/513/602/658/660/758, 391/451/472/513/557, 391/472/476/557/602, 391/472/513/557/602/658, 391/472/513/602/658, 391/476/513/743, 391/476/557, 391/513/658/660, 391/557, 391/557/758, 391/602, 391/602/758, 391/658, 391/660/743/758, 391/685, 396, 398, 398/472, 398/557/602/658, 412, 412/645, 412/674, 423, 423/476/605, 423/476/645/674, 423/645, 423/699, 425, 426, 435, 440, 455, 456, 467, 472, 472/476/513/743, 472/476/557/658/758, 472/513/557/658/743/758, 472/513/557/660/685, 472/513/658/660/743, 472/557/660/685/743/758, 472/758, 476, 476/557/758, 476/685, 476/758, 478/557, 479, 485, 506, 508, 513, 513/557/743, 513/557/758, 532, 533, 540, 557, 557/658/660, 557/658/685, 557/743, 564, 602, 602/685, 602/703, 605/699, 635, 643, 645, 645/659, 645/674/699, 658, 658/660, 658/685/758, 659, 660, 674, 704, 713, 718, 758, and 810, or combinations thereof, wherein the amino acid positions are relative to the reference sequence of SEQ ID NO: 2.

In some embodiments, the present invention provides an engineered polynucleotide encoding an engineered DNA polymerase comprising one or more substitutions or substitution sets selected from 11D/287L, 15S/633N, 18H/145H/255V, 20C/602H, 29N, 50H/255V/557R/685A, 110M, 113F/170L/194V/235E/249R/319V/645K/659P/660P/674V/699V, 130M, 145H, 145H/255V/287L/391S/472A/476L/557R, 145H/255V/287L/391S/476L/513T, 145H/255V/287L/391S/557R/685A/743T/758W, 145H/255V/391S/398R/472A/476L/513T/557R/602H/660S, 145H/255V/391S/398R/472A/557R/685A, 145H/255V/391S/398R/476L/557R/758W, 145H/255V/391S/398R/513T/658V/685A/743T, 145H/255V/391S/472A/476L/685A/743T, 145H/255V/391S/472A/557R/685A, 145H/255V/391S/476L/557R/602H/660S/743T, 145H/255V/391S/476L/557R/658V/660S/743T, 145H/255V/391S/476L/758W, 145H/255V/391S/513T/557R/743T/758W, 145H/255V/391S/557R/758W, 145H/255V/398R/472A/476L/513T/557R/658V/660S, 145H/255V/472A/476L/557R/685A/743T/758W, 145H/255V/472A/513T/557R/602H/660S/743T, 145H/255V/513T, 145H/255V/513T/602H, 145H/255V/557R, 145H/255V/557R/658V/660S/685A, 145H/255V/743T/758W, 145H/267S/391S/398R/513T/557R/658V/685A/743T, 145H/287L/391S/472A/557R/602H/658V/660S/685A, 145H/287L/391S/472A/602H/658V, 145H/287L/391S/476L/513T/557R/602H, 145H/287L/391S/476L/658V/660S/685A, 145H/287L/658V/758W, 145H/391S, 145H/391S/398R/476L/758W, 145H/391S/472A/476L/513T/557R/658V/685A/758W, 145H/391S/472A/476L/557R/602H/660S/685A/743T, 145H/391S/472A/476L/557R/743T/758W, 145H/391S/472A/476L/658V/660S/758W, 145H/391S/472A/476L/658V/685A/758W, 145H/391S/472A/476L/758W, 145H/391S/513T/557R/602H, 145H/391S/513T/557R/658V/685A, 145H/391S/513T/658V/743T/758W, 145H/391S/513T/758W, 145H/391S/557R, 145H/391S/557R/658V/758W, 145H/391S/557R/660S/685A, 145H/391S/557R/743T, 145H/398R/472A/557R/660S, 145H/476L/557R/660S/685A/743T/758W, 145H/513T/685A, 145H/557R/658V, 145H/557R/685A/758W, 145H/685A, 161V/162A, 161V/162A/170L/194V/235E/423Q/699V, 161V/162A/170L/235E/249R/252L, 161V/162A/194V/269R/605R/660P, 161V/162A/269R/4761/660P, 161V/162A/352K/434K/605R, 161V/162A/659P/660P/674V, 161V/170L/235E/249R/252L/434K, 161V/170L/235E/269R/319V, 161V/170L/235E/319V/412A, 161V/170L/252L/352K/412A/434K/699V, 161V/170L/476I, 161V/194V/198G/235E/252L/423Q/434K/605R/645K/699V, 161V/194V/235E/249R/352K/412A/434K/674V/699V, 161V/194V/252L, 161V/194V/252L/323S/352K/412A/605R/659P/660P/674V/699V, 161V/194V/252L/412A/605R, 161V/194V/434K, 161V/235E/249R/319V/412A/605R/659P, 161V/235E/252L/319V/352K/645K/674V/699V, 161V/235E/352K/423Q/645K/674V, 161V/235E/645K/699V, 161V/249R/412A/476I/605R/645K/659P/660P/674V, 161V/252L/319V/352K/412A/423Q/4761/605R/645K, 161V/252L/319V/674V, 161V/269R/434K/699V, 161V/319V/660P, 161V/352K, 161V/423Q/645K, 161V/605R, 162A, 162A/170L/194V/235E/249R/269R/674V/699V, 162A/170L/412A/434K/476I/605R, 162A/194V/249R/645K/659P, 162A/235E/269R/423Q/476I/605R/645K/659P, 162A/252L/352K/412A/434K/645K/660P/699V, 162A/269R/352K/412A/476I/605R, 162A/352K, 162A/423Q/645K, 162A/605R/645K, 162L, 164C, 168V, 170L, 170L/194V/198G/235E/674V/699V, 170L/194V/235E/252L/269R/323S/412A/423Q/476I/605R/645K/660P/674V, 170L/194V/235E/319V/412A/476I/699V, 170L/194V/269R/319V/412A/423Q/476I/605R/645K, 170L/194V/319V/352K/476I/699V, 170L/235E/249R/352K/645K, 170L/235E/269R/352K, 170L/235E/269R/434K/674V, 170L/235E/319V/352K/412A/434K/476I/645K/660P/674V, 170L/235E/352K/674V/699V, 170L/235E/352K/699V, 170L/235E/412A, 170L/235E/645K, 170L/235E/660P, 170L/249R/252L/269R/352K/412A/423Q/434K/476I/659P, 170L/249R/252L/269R/423Q/659P, 170L/249R/412A, 170L/252L/352K/423Q/674V/699V, 170L/269R/352K/423Q/4761/605R/659P/660P/699V, 170L/269R/476I/645K, 170L/412A/423Q/476I/605R/645K/660P/674V, 170L/4761/674V, 175A, 177F, 194V, 194V/198G/235E/252L/323S/412A/605R/645K, 194V/198G/252L/319V/423Q/645K/674V, 194V/235E/252L, 194V/235E/252L/319V/423Q, 194V/235E/605R, 194V/235E/645K, 194V/235E/699V, 194V/249R/252L/645K/699V, 194V/249R/412A/476I/605R/645K/699V, 194V/252L/269R/319V/434K/605R/645K/659P/674V/699V, 194V/252L/319V/412A/605R, 194V/269R/319V/412A/605R/674V/699V, 194V/319V/352K/605R/660P, 194V/323S/699V, 194V/352K/476I/659P, 194V/352K/605R/645K/699V, 194V/434K, 194V/645K/659P, 197Q, 198G/674V/699V, 202S/268K, 217T, 227E, 235E, 235E/249R/252L/323S/352K/476I, 235E/249R/252L/412A/605R/645K/699V, 235E/249R/252L/423Q/699V, 235E/249R/252L/434K/605R/645K/699V, 235E/252L, 235E/252L/269R/352K/434K/605R/674V/699V, 235E/252L/269R/352K/4761/645K/674V, 235E/252L/269R/423Q/605R/645K/660P/674V, 235E/269R/323S/412A/434K/659P/674V/699V, 235E/269R/674V/699V, 235E/319V/605R/645K/674V, 235E/352K, 235E/352K/412A/659P/660P/674V/699V, 235E/352K/605R, 235E/412A/645K, 235E/476I/674V, 235E/605R/645K, 249R/252L/319V/352K/645K, 249R/252L/319V/605R/660P, 249R/252L/412A/423Q/645K/674V/699V, 249R/412A/476I/605R, 252L/352K/645K/660P, 252L/645K, 253D, 255V/260A/398R/557R, 255V/287L, 255V/287L/391S/472A/557R/658V/660S, 255V/287L/391S/472A/658V/660S/685A, 255V/287L/391S/476L/557R, 255V/287L/391S/658V, 255V/391S, 255V/391S/398R/476L/557R, 255V/391S/398R/476L/602H/658V/660S/685A, 255V/391S/398R/513T/557R/658V/743T/758W, 255V/391S/472A/513T/758W, 255V/391S/472A/557R/658V/660S/685A/743T/758W, 255V/391S/472A/557R/658V/660S/758W, 255V/391S/472A/658V/660S/758W, 255V/391S/472A/685A, 255V/391S/476L/513T/557R/602H/685A/758W, 255V/391S/476L/557R/602H/660S/758W, 255V/391S/476L/557R/658V, 255V/391S/476L/758W, 255V/391S/513T/602H/658V/660S, 255V/391S/557R, 255V/391S/602H, 255V/391S/685A/758W, 255V/391S/743T, 255V/398R/472A/476L/557R/658V, 255V/398R/476L, 255V/398R/476L/557R/658V/660S/685A, 255V/398R/513T/758W, 255V/472A/758W, 255V/513T/557R, 255V/513T/557R/685A/758W, 255V/557R/658V, 255V/602H, 255V/660S, 267Y, 268K, 269R/352K, 269R/352K/412A, 269R/605R, 270Y, 271H/472A, 2791, 281M, 287L/391S/472A, 287L/391S/472A/476L/557R/602H/658V/660S/685A/743T, 287L/391S/476L/513T, 287L/391S/513T/557R/660S/685A/743T/758W, 287L/391S/557R, 287L/391S/557R/602H/658V/743T, 287L/391S/557R/685A, 287L/391S/602H, 287L/685A, 297T, 319V, 319V/352K/412A/434K/699V, 319V/434K/476I, 322V, 329Q, 331S, 339E, 3471, 350F, 352G, 352K, 352K/412A/605R/645K/699V, 352K/605R/645K/699V, 352K/605R/659P, 352K/645K, 354I, 356N, 358N, 360F, 361I, 3651, 372N, 377Q, 3791, 391S, 391S/398R/476L/685A, 391S/398R/513T/602H/658V/660S/758W, 391S/451V/472A/513T/557R, 391S/472A/476L/557R/602H, 391S/472A/513T/557R/602H/658V, 391S/472A/513T/602H/658V, 391S/476L/513T/743T, 391S/476L/557R, 391S/513T/658V/660S, 391S/557R, 391S/557R/758W, 391S/602H, 391S/602H/758W, 391S/658V, 391S/660S/743T/758W, 391S/685A, 396K, 398R, 398R/472A, 398R/557R/602H/658V, 398T, 412A, 412A/645K, 412A/674V, 423Q, 423Q/476I/605R, 423Q/4761/645K/674V, 423Q/645K, 423Q/699V, 423V, 425F, 426V, 435Y, 440T, 455V, 456K, 467N, 472A, 472A/476L/513T/743T, 472A/476L/557R/658V/758W, 472A/513T/557R/658V/743T/758W, 472A/513T/557R/660S/685A, 472A/513T/658V/660S/743T, 472A/557R/660S/685A/743T/758W, 472A/758W, 476I, 476L, 476L/557R/758W, 476L/685A, 476L/758W, 478H/557R, 479M, 485K, 506N, 508N, 513T, 513T/557R/743T, 513T/557R/758W, 532M, 533D, 540N, 557R, 557R/658V/660S, 557R/658V/685A, 557R/743T, 564E, 602H, 602H/685A, 602H/703R, 605R/699V, 635K, 643H, 645K, 645K/659P, 645K/674V/699V, 658V, 658V/660S, 658V/685A/758W, 659E, 660S, 674V, 704K, 713Q, 718D, 758W, and 810K, or combinations thereof, wherein the amino acid positions are relative to SEQ ID NO: 2.

In some embodiments, the present invention provides an engineered polynucleotide encoding an engineered DNA polymerase comprising one or more substitutions or substitution sets are selected from G11D/F287L, L15S/G633N, P18H/Q145H/S255V, G20C/K602H, H29N, Q50H/S255V/P557R/H685A, L110M, L113F/R170L/A194V/A235E/K249R/R319V/R645K/S659P/A660P/1674V/A699V, L130M, Q145H, Q145H/S255V/F287L/P391S/V472A/M476L/P557R, Q145H/S255V/F287L/P391S/M476L/G513T, Q145H/S255V/F287L/P391S/P557R/H685A/N743T/F758W, Q145H/S255V/P391S/G398R/V472A/M476L/G513T/P557R/K602H/A660S, Q145H/S255V/P391S/G398R/V472A/P557R/H685A, Q145H/S255V/P391S/G398R/M476L/P557R/F758W, Q145H/S255V/P391S/G398R/G513T/L658V/H685A/N743T, Q145H/S255V/P391S/V472A/M476L/H685A/N743T, Q145H/S255V/P391S/V472A/P557R/H685A, Q145H/S255V/P391S/M476L/P557R/K602H/A660S/N743T, Q145H/S255V/P391S/M476L/P557R/L658V/A660S/N743T, Q145H/S255V/P391S/M476L/F758W, Q145H/S255V/P391S/G513T/P557R/N743T/F758W, Q145H/S255V/P391S/P557R/F758W, Q145H/S255V/G398R/V472A/M476L/G513T/P557R/L658V/A660S, Q145H/S255V/V472A/M476L/P557R/H685A/N743T/F758W, Q145H/S255V/V472A/G513T/P557R/K602H/A660S/N743T, Q145H/S255V/G513T, Q145H/S255V/G513T/K602H, Q145H/S255V/P557R, Q145H/S255V/P557R/L658V/A660S/H685A, Q145H/S255V/N743T/F758W, Q145H/F267S/P391S/G398R/G513T/P557R/L658V/H685A/N743T, Q145H/F287L/P391S/V472A/P557R/K602H/L658V/A660S/H685A, Q145H/F287L/P391S/V472A/K602H/L658V, Q145H/F287L/P391S/M476L/G513T/P557R/K602H, Q145H/F287L/P391S/M476L/L658V/A660S/H685A, Q145H/F287L/L658V/F758W, Q145H/P391S, Q145H/P391S/G398R/M476L/F758W, Q145H/P391S/V472A/M476L/G513T/P557R/L658V/H685A/F758W, Q145H/P391S/V472A/M476L/P557R/K602H/A660S/H685A/N743T, Q145H/P391S/V472A/M476L/P557R/N743T/F758W, Q145H/P391S/V472A/M476L/L658V/A660S/F758W, Q145H/P391S/V472A/M476L/L658V/H685A/F758W, Q145H/P391S/V472A/M476L/F758W, Q145H/P391S/G513T/P557R/K602H, Q145H/P391S/G513T/P557R/L658V/H685A, Q145H/P391S/G513T/L658V/N743T/F758W, Q145H/P391S/G513T/F758W, Q145H/P391S/P557R, Q145H/P391S/P557R/L658V/F758W, Q145H/P391S/P557R/A660S/H685A, Q145H/P391S/P557R/N743T, Q145H/G398R/V472A/P557R/A660S, Q145H/M476L/P557R/A660S/H685A/N743T/F758W, Q145H/G513T/H685A, Q145H/P557R/L658V, Q145H/P557R/H685A/F758W, Q145H/H685A, I161V/S162A, I161V/S162A/R170L/A194V/A235E/A423Q/A699V, I161V/S162A/R170L/A235E/K249R/R252L, I161V/S162A/A194V/G269R/K605R/A660P, I161V/S162A/G269R/M476I/A660P, I161V/S162A/R352K/R434K/K605R, I161V/S162A/S659P/A660P/1674V, I161V/R170L/A235E/K249R/R252L/R434K, I161V/R170L/A235E/G269R/R319V, I161V/R170L/A235E/R319V/G412A, I161V/R170L/R252L/R352K/G412A/R434K/A699V, I161V/R170L/M476I, I161V/A194V/S198G/A235E/R252L/A423Q/R434K/K605R/R645K/A699V, I161V/A194V/A235E/K249R/R352K/G412A/R434K/1674V/A699V, I161V/A194V/R252L, I161V/A194V/R252L/P323S/R352K/G412A/K605R/S659P/A660P/1674V/A699V, I161V/A194V/R252L/G412A/K605R, I161V/A194V/R434K, I161V/A235E/K249R/R319V/G412A/K605R/S659P, I161V/A235E/R252L/R319V/R352K/R645K/1674V/A699V, I161V/A235E/R352K/A423Q/R645K/1674V, I161V/A235E/R645K/A699V, I161V/K249R/G412A/M476I/K605R/R645K/S659P/A660P/1674V, I161V/R252L/R319V/R352K/G412A/A423Q/M476I/K605R/R645K, I161V/R252L/R319V/1674V, I161V/G269R/R434K/A699V, I161V/R319V/A660P, I161V/R352K, I161V/A423Q/R645K, I161V/K605R, S162A, S162A/R170L/A194V/A235E/K249R/G269R/1674V/A699V, S162A/R170L/G412A/R434K/M476I/K605R, S162A/A194V/K249R/R645K/S659P, S162A/A235E/G269R/A423Q/M476I/K605R/R645K/S659P, S162A/R252L/R352K/G412A/R434K/R645K/A660P/A699V, S162A/G269R/R352K/G412A/M476I/K605R, S162A/R352K, S162A/A423Q/R645K, S162A/K605R/R645K, S162L, L164C, G168V, R170L, R170L/A194V/S198G/A235E/1674V/A699V, R170L/A194V/A235E/R252L/G269R/P323S/G412A/A423Q/M476I/K605R/R645K/A660P/1674V, R170L/A194V/A235E/R319V/G412A/M476I/A699V, R170L/A194V/G269R/R319V/G412A/A423Q/M476I/K605R/R645K, R170L/A194V/R319V/R352K/M476I/A699V, R170L/A235E/K249R/R352K/R645K, R170L/A235E/G269R/R352K, R170L/A235E/G269R/R434K/1674V, R170L/A235E/R319V/R352K/G412A/R434K/M476I/R645K/A660P/1674V, R170L/A235E/R352K/1674V/A699V, R170L/A235E/R352K/A699V, R170L/A235E/G412A, R170L/A235E/R645K, R170L/A235E/A660P, R170L/K249R/R252L/G269R/R352K/G412A/A423Q/R434K/M476I/S659P, R170L/K249R/R252L/G269R/A423Q/S659P, R170L/K249R/G412A, R170L/R252L/R352K/A423Q/1674V/A699V, R170L/G269R/R352K/A423Q/M476I/K605R/S659P/A660P/A699V, R170L/G269R/M476I/R645K, R170L/G412A/A423Q/M476I/K605R/R645K/A660P/1674V, R170L/M476I/1674V, W175A, W177F, A194V, A194V/S198G/A235E/R252L/P323S/G412A/K605R/R645K, A194V/S198G/R252L/R319V/A423Q/R645K/1674V, A194V/A235E/R252L, A194V/A235E/R252L/R319V/A423Q, A194V/A235E/K605R, A194V/A235E/R645K, A194V/A235E/A699V, A194V/K249R/R252L/R645K/A699V, A194V/K249R/G412A/M476I/K605R/R645K/A699V, A194V/R252L/G269R/R319V/R434K/K605R/R645K/S659P/1674V/A699V, A194V/R252L/R319V/G412A/K605R, A194V/G269R/R319V/G412A/K605R/1674V/A699V, A194V/R319V/R352K/K605R/A660P, A194V/P323S/A699V, A194V/R352K/M476I/S659P, A194V/R352K/K605R/R645K/A699V, A194V/R434K, A194V/R645K/S659P, P197Q, S198G/1674V/A699V, P202S/R268K, K217T, K227E, A235E, A235E/K249R/R252L/P323S/R352K/M476I, A235E/K249R/R252L/G412A/K605R/R645K/A699V, A235E/K249R/R252L/A423Q/A699V, A235E/K249R/R252L/R434K/K605R/R645K/A699V, A235E/R252L, A235E/R252L/G269R/R352K/R434K/K605R/1674V/A699V, A235E/R252L/G269R/R352K/M476I/R645K/1674V, A235E/R252L/G269R/A423Q/K605R/R645K/A660P/1674V, A235E/G269R/P323S/G412A/R434K/S659P/1674V/A699V, A235E/G269R/1674V/A699V, A235E/R319V/K605R/R645K/1674V, A235E/R352K, A235E/R352K/G412A/S659P/A660P/1674V/A699V, A235E/R352K/K605R, A235E/G412A/R645K, A235E/M476I/1674V, A235E/K605R/R645K, K249R/R252L/R319V/R352K/R645K, K249R/R252L/R319V/K605R/A660P, K249R/R252L/G412A/A423Q/R645K/1674V/A699V, K249R/G412A/M476I/K605R, R252L/R352K/R645K/A660P, R252L/R645K, E253D, S255V/D260A/G398R/P557R, S255V/F287L, S255V/F287L/P391S/V472A/P557R/L658V/A660S, S255V/F287L/P391S/V472A/L658V/A660S/H685A, S255V/F287L/P391S/M476L/P557R, S255V/F287L/P391S/L658V, S255V/P391S, S255V/P391S/G398R/M476L/P557R, S255V/P391S/G398R/M476L/K602H/L658V/A660S/H685A, S255V/P391S/G398R/G513T/P557R/L658V/N743T/F758W, S255V/P391S/V472A/G513T/F758W, S255V/P391S/V472A/P557R/L658V/A660S/H685A/N743T/F758W, S255V/P391S/V472A/P557R/L658V/A660S/F758W, S255V/P391S/V472A/L658V/A660S/F758W, S255V/P391S/V472A/H685A, S255V/P391S/M476L/G513T/P557R/K602H/H685A/F758W, S255V/P391S/M476L/P557R/K602H/A660S/F758W, S255V/P391S/M476L/P557R/L658V, S255V/P391S/M476L/F758W, S255V/P391S/G513T/K602H/L658V/A660S, S255V/P391S/P557R, S255V/P391S/K602H, S255V/P391S/H685A/F758W, S255V/P391S/N743T, S255V/G398R/V472A/M476L/P557R/L658V, S255V/G398R/M476L, S255V/G398R/M476L/P557R/L658V/A660S/H685A, S255V/G398R/G513T/F758W, S255V/V472A/F758W, S255V/G513T/P557R, S255V/G513T/P557R/H685A/F758W, S255V/P557R/L658V, S255V/K602H, S255V/A660S, F267Y, R268K, G269R/R352K, G269R/R352K/G412A, G269R/K605R, R270Y, R271H/V472A, K2791, F281M, F287L/P391S/V472A, F287L/P391S/V472A/M476L/P557R/K602H/L658V/A660S/H685A/N743T, F287L/P391S/M476L/G513T, F287L/P391S/G513T/P557R/A660S/H685A/N743T/F758W, F287L/P391S/P557R, F287L/P391S/P557R/K602H/L658V/N743T, F287L/P391S/P557R/H685A, F287L/P391S/K602H, F287L/H685A, L297T, R319V, R319V/R352K/G412A/R434K/A699V, R319V/R434K/M476I, R322V, E329Q, L331S, G339E, P347I, A350F, R352G, R352K, R352K/G412A/K605R/R645K/A699V, R352K/K605R/R645K/A699V, R352K/K605R/S659P, R352K/R645K, L354I, E356N, R358N, L360F, L361I, L365I, E372N, P377Q, T379I, P391S, P391S/G398R/M476L/H685A, P391S/G398R/G513T/K602H/L658V/A660S/F758W, P391S/A451V/V472A/G513T/P557R, P391S/V472A/M476L/P557R/K602H, P391S/V472A/G513T/P557R/K602H/L658V, P391S/V472A/G513T/K602H/L658V, P391S/M476L/G513T/N743T, P391S/M476L/P557R, P391S/G513T/L658V/A660S, P391S/P557R, P391S/P557R/F758W, P391S/K602H, P391S/K602H/F758W, P391S/L658V, P391S/A660S/N743T/F758W, P391S/H685A, P396K, G398R, G398R/V472A, G398R/P557R/K602H/L658V, G398T, G412A, G412A/R645K, G412A/1674V, A423Q, A423Q/M476I/K605R, A423Q/M476I/R645K/1674V, A423Q/R645K, A423Q/A699V, A423V, L425F, L426V, L435Y, E440T, A455V, T456K, A467N, V472A, V472A/M476L/G513T/N743T, V472A/M476L/P557R/L658V/F758W, V472A/G513T/P557R/L658V/N743T/F758W, V472A/G513T/P557R/A660S/H685A, V472A/G513T/L658V/A660S/N743T, V472A/P557R/A660S/H685A/N743T/F758W, V472A/F758W, M476I, M476L, M476L/P557R/F758W, M476L/H685A, M476L/F758W, R478H/P557R, L479M, R485K, E506N, G508N, G513T, G513T/P557R/N743T, G513T/P557R/F758W, R532M, E533D, K540N, P557R, P557R/L658V/A660S, P557R/L658V/H685A, P557R/N743T, P564E, K602H, K602H/H685A, K602H/Q703R, K605R/A699V, E635K, E643H, R645K, R645K/S659P, R645K/1674V/A699V, L658V, L658V/A660S, L658V/H685A/F758W, S659E, A660S, 1674V, R704K, K713Q, K718D, F758W, and A810K, or combinations thereof, wherein the amino acid positions are relative to the reference sequence of SEQ ID NO: 2.

In some embodiments, the present invention provides an engineered polynucleotide encoding an engineered DNA polymerase comprising at least one or more substitutions or substitution sets at amino acid positions selected from 11/287, 29, 50/255/557/685, 145/255/287/391/472/476/557, 145/255/391/472/557/685, 145/255/391/685, 145/255/472/476/557/685/743/758, 145/255/557/658/660/685, 145/391/472/476/513/557/658/685/758, 145/391/513/557/602, 145/391/557/743, 145/398/472/557/660, 145/557/658, 145/743, 161/162/269/476/660, 161/162/659/660/674, 161/170/235/269/319, 161/170/235/319/412, 161/194/235/249/352/412/434/674/699, 161/194/252/412/605, 161/235/252/319/352/645/674/699, 161/235/645/699, 161/249/412/476/605/645/659/660/674, 161/252/319/352/412/423/476/605/645, 161/319/660, 161/352, 162, 162/352, 162/423/645, 170, 170/194/235/252/269/323/412/423/476/605/645/660/674, 170/235/249/352/645, 170/235/319/352/412/434/476/645/660/674, 170/235/352/674/699, 170/235/352/699, 170/235/412, 170/235/645, 170/235/660, 170/252/352/423/674/699, 170/269/352/423/476/605/659/660/699, 194, 194/198/252/319/423/645/674, 194/235/252, 194/252/269/319/434/605/645/659/674/699, 194/269/319/412/605/674/699, 194/319/352/605/660, 194/352/605/645/699, 194/645/659, 197, 217, 227, 235/249/252/323/352/476, 235/249/252/423/699, 235/252, 235/252/269/423/605/645/660/674, 235/269/323/412/434/659/674/699, 235/352, 235/352/605, 235/605/645, 249/252/319/605/660, 252/645, 255, 255/391/472/557/660/743, 255/391/476/513/557/602/685/758, 255/391/476/557/658, 255/391/557, 255/398/472/476/557/658, 255/398/476/557/658/660/685, 255/476, 255/660, 268, 269/352, 269/352/412, 269/605, 319, 319/352/412/434/699, 319/434/476, 329, 331, 339, 352, 352/412/605/645/699, 354, 358, 360, 391, 391/398/476/685, 391/451/472/513/557, 391/472/476/557/602, 391/472/476/557/658/660/743/758, 391/557, 391/581, 412, 412/645, 412/674, 423/476/645/674, 423/645, 467, 478/557, 506, 540, 557, 557/658/660, 557/658/685, 602/701, 602/703, 605/699, 658, 658/660, 660, 699, 713, and 743, wherein the amino acid positions are relative to the reference sequence of SEQ ID NO: 2.

In some embodiments, the present invention provides an engineered polynucleotide encoding an engineered DNA polymerase comprising one or more substitutions or substitution sets selected from 11D/287L, 29N, 50H/255V/557R/685A, 145H/255V/287L/391S/472A/476L/557R, 145H/255V/391S/472A/557R/685A, 145H/255V/391S/685A, 145H/255V/472A/476L/557R/685A/743T/758W, 145H/255V/557R/658V/660S/685A, 145H/391S/472A/476L/513T/557R/658V/685A/758W, 145H/391S/513T/557R/602H, 145H/391S/557R/743T, 145H/398R/472A/557R/660S, 145H/557R/658V, 145H/743T, 161V/162A/269R/476I/660P, 161V/162A/659P/660P/674V, 161V/170L/235E/269R/319V, 161V/170L/235E/319V/412A, 161V/194V/235E/249R/352K/412A/434K/674V/699V, 161V/194V/252L/412A/605R, 161V/235E/252L/319V/352K/645K/674V/699V, 161V/235E/645K/699V, 161V/249R/412A/476I/605R/645K/659P/660P/674V, 161V/252L/319V/352K/412A/423Q/476I/605R/645K, 161V/319V/660P, 161V/352K, 162A, 162A/352K, 162A/423Q/645K, 170L, 170L/194V/235E/252L/269R/323S/412A/423Q/476I/605R/645K/660P/674V, 170L/235E/249R/352K/645K, 170L/235E/319V/352K/412A/434K/476I/645K/660P/674V, 170L/235E/352K/674V/699V, 170L/235E/352K/699V, 170L/235E/412A, 170L/235E/645K, 170L/235E/660P, 170L/252L/352K/423Q/674V/699V, 170L/269R/352K/423Q/4761/605R/659P/660P/699V, 194V, 194V/198G/252L/319V/423Q/645K/674V, 194V/235E/252L, 194V/252L/269R/319V/434K/605R/645K/659P/674V/699V, 194V/269R/319V/412A/605R/674V/699V, 194V/319V/352K/605R/660P, 194V/352K/605R/645K/699V, 194V/645K/659P, 197Q, 217T, 227E, 235E/249R/252L/323S/352K/476I, 235E/249R/252L/423Q/699V, 235E/252L, 235E/252L/269R/423Q/605R/645K/660P/674V, 235E/269R/323S/412A/434K/659P/674V/699V, 235E/352K, 235E/352K/605R, 235E/605R/645K, 249R/252L/319V/605R/660P, 252L/645K, 255V, 255V/391S/472A/557R/660S/743T, 255V/391S/476L/513T/557R/602H/685A/758W, 255V/391S/476L/557R/658V, 255V/391S/557R, 255V/398R/472A/476L/557R/658V, 255V/398R/476L/557R/658V/660S/685A, 255V/476L, 255V/660S, 268K, 269R/352K, 269R/352K/412A, 269R/605R, 319V, 319V/352K/412A/434K/699V, 319V/434K/4761, 329Q, 331S, 339E, 352G, 352K, 352K/412A/605R/645K/699V, 3541, 358N, 360F, 391S, 391S/398R/476L/685A, 391S/451V/472A/513T/557R, 391S/472A/476L/557R/602H, 391S/472A/476L/557R/658V/660S/743T/758W, 391S/557R, 391S/581D, 412A, 412A/645K, 412A/674V, 423Q/4761/645K/674V, 423Q/645K, 467N, 478H/557R, 506N, 540N, 557R, 557R/658V/660S, 557R/658V/685A, 602H/701L, 602H/703R, 605R/699V, 658V, 658V/660S, 660S, 699V, 713Q, and 743T, wherein the amino acid positions are relative to the reference sequence of SEQ ID NO: 2.

In some embodiments, the present invention provides an engineered polynucleotide encoding an engineered DNA polymerase comprising one or more substitutions or substitution sets selected from G11D/F287L, H29N, Q50H/S255V/P557R/H685A, Q145H/S255V/F287L/P391S/V472A/M476L/P557R, Q145H/S255V/P391S/V472A/P557R/H685A, Q145H/S255V/P391S/H685A, Q145H/S255V/V472A/M476L/P557R/H685A/N743T/F758W, Q145H/S255V/P557R/L658V/A660S/H685A, Q145H/P391S/V472A/M476L/G513T/P557R/L658V/H685A/F758W, Q145H/P391S/G513T/P557R/K602H, Q145H/P391S/P557R/N743T, Q145H/G398R/V472A/P557R/A660S, Q145H/P557R/L658V, Q145H/N743T, I161V/S162A/G269R/M476I/A660P, I161V/S162A/S659P/A660P/1674V, I161V/R170L/A235E/G269R/R319V, I161V/R170L/A235E/R319V/G412A, I161V/A194V/A235E/K249R/R352K/G412A/R434K/1674V/A699V, I161V/A194V/R252L/G412A/K605R, I161V/A235E/R252L/R319V/R352K/R645K/1674V/A699V, I161V/A235E/R645K/A699V, I161V/K249R/G412A/M476I/K605R/R645K/S659P/A660P/1674V, I161V/R252L/R319V/R352K/G412A/A423Q/M476I/K605R/R645K, I161V/R319V/A660P, I161V/R352K, S162A, S162A/R352K, S162A/A423Q/R645K, R170L, R170L/A194V/A235E/R252L/G269R/P323S/G412A/A423Q/M476I/K605R/R645K/A660P/1674V, R170L/A235E/K249R/R352K/R645K, R170L/A235E/R319V/R352K/G412A/R434K/M476I/R645K/A660P/1674V, R170L/A235E/R352K/1674V/A699V, R170L/A235E/R352K/A699V, R170L/A235E/G412A, R170L/A235E/R645K, R170L/A235E/A660P, R170L/R252L/R352K/A423Q/1674V/A699V, R170L/G269R/R352K/A423Q/M476I/K605R/S659P/A660P/A699V, A194V, A194V/S198G/R252L/R319V/A423Q/R645K/1674V, A194V/A235E/R252L, A194V/R252L/G269R/R319V/R434K/K605R/R645K/S659P/1674V/A699V, A194V/G269R/R319V/G412A/K605R/1674V/A699V, A194V/R319V/R352K/K605R/A660P, A194V/R352K/K605R/R645K/A699V, A194V/R645K/S659P, P197Q, K217T, K227E, A235E/K249R/R252L/P323S/R352K/M476I, A235E/K249R/R252L/A423Q/A699V, A235E/R252L, A235E/R252L/G269R/A423Q/K605R/R645K/A660P/1674V, A235E/G269R/P323S/G412A/R434K/S659P/1674V/A699V, A235E/R352K, A235E/R352K/K605R, A235E/K605R/R645K, K249R/R252L/R319V/K605R/A660P, R252L/R645K, S255V, S255V/P391S/V472A/P557R/A660S/N743T, S255V/P391S/M476L/G513T/P557R/K602H/H685A/F758W, S255V/P391S/M476L/P557R/L658V, S255V/P391S/P557R, S255V/G398R/V472A/M476L/P557R/L658V, S255V/G398R/M476L/P557R/L658V/A660S/H685A, S255V/M476L, S255V/A660S, R268K, G269R/R352K, G269R/R352K/G412A, G269R/K605R, R319V, R319V/R352K/G412A/R434K/A699V, R319V/R434K/M476I, E329Q, L331S, G339E, R352G, R352K, R352K/G412A/K605R/R645K/A699V, L354I, R358N, L360F, P391S, P391S/G398R/M476L/H685A, P391S/A451V/V472A/G513T/P557R, P391S/V472A/M476L/P557R/K602H, P391S/V472A/M476L/P557R/L658V/A660S/N743T/F758W, P391S/P557R, P391S/G581D, G412A, G412A/R645K, G412A/1674V, A423Q/M476I/R645K/1674V, A423Q/R645K, A467N, R478H/P557R, E506N, K540N, P557R, P557R/L658V/A660S, P557R/L658V/H685A, K602H/F701L, K602H/Q703R, K605R/A699V, L658V, L658V/A660S, A660S, A699V, K713Q, and N743T, wherein the amino acid positions are relative to the reference sequence of SEQ ID NO: 2.

In some additional embodiments, the present invention provides an engineered polynucleotide encoding a DNA polymerase or a functional fragment thereof, comprising a polypeptide sequence having at least 75% or more sequence identity to a reference sequence comprising residues 12 to 841 of SEQ ID NO: 178, or to a reference sequence of SEQ ID NO: 178, wherein the polypeptide sequence comprises one or more substitutions relative to the reference sequence comprising residues 12 to 841 of SEQ ID NO: 178, or to the reference sequence of SEQ ID NO: 178.

In some embodiments, the engineered polypeptide encodes an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 178 or to the reference sequence of SEQ ID NO: 178.

In some embodiments, the present invention provides an engineered polynucleotide encoding an engineered DNA polymerase comprising one or more substitutions or substitution sets selected from 12, 14, 15, 17, 19, 36, 39, 44, 45, 82, 86, 87, 95, 122, 139, 139/395, 159, 167, 170/194, 170/194/423/451/557/658/660/743, 170/194/423/476/743, 170/194/423/557/658/659/743, 170/194/423/557/658/660/685, 170/194/423/557/658/743, 170/194/451/476/557/658, 170/194/451/476/557/658/659/660/743, 170/194/451/476/557/658/685, 170/194/451/557/659/660, 170/194/476/557/659, 170/194/557, 170/194/658, 170/194/658/659/660/685/743, 170/194/658/743, 170/423, 170/423/451/476/557/658/659/743, 170/423/451/557, 170/423/451/557/658/659/685/743, 170/423/451/557/659, 170/423/476, 170/423/476/658/659, 170/423/476/658/659/660/685/743, 170/423/476/658/660/685/743, 170/423/476/658/685, 170/423/476/658/685/743, 170/423/476/685, 170/423/476/685/743, 170/423/557/658/659, 170/423/557/658/660/743, 170/423/557/658/743, 170/423/557/659/660/685/743, 170/423/557/660/743, 170/423/557/685, 170/423/557/685/743, 170/423/658, 170/423/658/659/743, 170/423/658/660, 170/423/743, 170/451/557, 170/451/557/658/660, 170/476/557/658/659, 170/476/557/658/685, 170/476/557/743, 170/476/659, 170/557, 170/557/658/660/685/687, 170/658, 170/658/659, 170/658/659/743, 170/659, 170/660, 179, 194/423/451/557/658/659/743, 194/423/476/658/660/685, 194/423/557/658/659/743, 194/423/685, 194/451/476/557/658/659, 194/451/476/557/658/659/660/743, 194/557, 194/557/658/659/685, 194/557/659/743, 194/658/659/685, 194/658/660, 197, 198, 198/423/476/557/743, 205, 209, 214, 222, 228, 249, 250/269/331, 252, 252/258/331, 252/258/331/352/467/506/674, 252/269, 252/269/331/339/412/413, 252/269/331/352/412/674/704, 252/269/331/506/674, 252/269/339/352/356/660/704, 252/269/339/352/412/699/704, 252/269/352/660, 252/269/412/674, 252/269/413/467/660/699/704, 252/269/467/506, 252/269/674, 252/331, 252/331/339/352/467/506, 252/331/339/412/413/467/699, 252/331/352/412/413/467/660/704, 252/331/352/412/506/674, 252/331/358/506, 252/331/412/413, 252/331/412/413/674, 252/331/412/467/506/699/704, 252/331/412/506/674, 252/331/412/506/704, 252/331/413, 252/331/467/660/674, 252/331/660, 252/331/699, 252/339/352/467/674/699, 252/339/352/674/704, 252/339/412/413/467/674, 252/339/412/413/674/699/704, 252/339/412/467/674, 252/339/412/674/704, 252/339/467/506/699/704, 252/341/412/413, 252/341/412/674, 252/352/412, 252/352/506, 252/352/660, 252/358/674, 252/412, 252/412/413, 252/412/467, 252/412/467/660/699/704, 252/413, 252/413/506/660/704, 252/413/699, 252/467, 252/467/506/674/704, 252/506/704, 252/674, 252/699, 252/704, 264, 268, 268/510, 268/621, 269, 269/331/352/412/467/506/704, 269/331/352/467/506/699, 269/331/352/467/699, 269/331/412/413/467/506/660/674, 269/331/413/506, 269/331/467/506/660, 269/331/660/699, 269/331/674/699, 269/331/699, 269/331/699/704, 269/339/506/704, 269/352/467/506/674, 269/358, 269/358/412/660/704, 269/358/467/506/660/699, 269/412/413/506/704, 269/412/413/674, 269/412/467/704, 269/467/506, 269/506, 269/599/660/674/699, 269/674, 269/699/704, 269/704, 272/352/506, 275, 301, 323, 331, 331/339/412/413/467/506/674/704, 331/339/412/467/506/704, 331/352/358/699/704, 331/352/412/413/506, 331/358, 331/358/467/674/699/704, 331/412, 331/412/413/467/699/704, 331/412/467/674/704, 331/412/467/704, 331/412/660/699, 331/413/467/660/674, 331/467, 331/467/506, 331/467/674/699, 331/506, 331/506/674/699, 331/660/674/699, 331/674, 331/674/699, 331/699, 331/704, 339, 339/352, 339/352/412/419/467/506/660/674/699, 339/352/412/467/660, 339/352/412/660/674, 339/352/674/699, 339/412/413, 339/412/413/467/674/699, 339/412/467/674, 339/467/674, 339/506, 339/506/699, 339/506/704, 339/660, 339/660/674, 341/352/704, 341/467, 341/467/506/660/674, 341/467/674, 345, 352, 352/358, 352/412/413/467/506/699, 352/413/467/506, 352/413/674, 352/660, 352/674, 355, 358/412, 358/413, 358/674, 412, 412/413/467/660, 412/413/467/674, 412/467/506/674/704, 412/467/660/674/699, 412/660, 412/674, 412/674/699, 413/467, 413/506, 413/506/704, 413/674/699/704/740, 423, 423/451/476/557/658/685, 423/451/476/658/685, 423/451/557, 423/451/557/658, 423/451/557/658/685, 423/476, 423/476/557/658/659/660, 423/476/557/658/659/685/743, 423/476/557/659/660/685/743, 423/476/557/659/660/743, 423/476/658/660/743, 423/476/658/685/743, 423/476/658/743, 423/557/658, 423/557/658/659/660, 423/557/658/659/660/685, 423/557/658/660/743, 423/557/685, 423/557/685/743, 423/658, 423/658/659/660, 423/658/660, 423/660, 423/685, 423/685/743, 423/743, 428, 433, 433/497, 467, 467/506/699/704, 467/658/660/674/699, 470, 476, 476/480/557/659, 476/557/658/659, 476/557/658/660/685, 476/557/659, 476/557/659/660, 476/658/659/660/685/743, 476/658/660/743, 477, 478, 485, 489/557/743, 492, 501, 506/660, 506/674/699/704, 506/674/704, 511, 514, 517, 520, 525/557/659/660/743, 533, 534, 534/670, 549, 557, 557/658, 557/658/659, 557/658/659/660, 557/659/660, 557/660, 557/743, 565, 596, 598, 642, 658, 658/659, 658/659/660/685/743, 658/659/685, 658/743, 659/660, 659/660/685/743, 674, 674/699, 685, 689, 695, 696, 699, 699/704, 704, 707, 725, 743, 745, 747, 783, and 821, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets are selected from 12E, 12L, 12S, 12V, 14E, 14V, 15V, 17S, 19A, 19S, 36H, 39E, 39P, 39V, 44A, 44L, 45L, 45S, 82K, 86G, 87A, 87P, 87R, 95D, 95G, 95R, 122A, 122Q, 139D/395A, 139S, 159Q, 167D, 170L/194V, 170L/194V/423Q/451H/557R/658V/660S/743T, 170L/194V/423Q/476L/743T, 170L/194V/423Q/557R/658V/659P/743T, 170L/194V/423Q/557R/658V/660S/685A, 170L/194V/423Q/557R/658V/743T, 170L/194V/451H/476L/557R/658V, 170L/194V/451H/476L/557R/658V/659P/660S/743T, 170L/194V/451H/476L/557R/658V/685A, 170L/194V/451H/557R/659P/660S, 170L/194V/476L/557R/659P, 170L/194V/557R, 170L/194V/658V, 170L/194V/658V/659P/660S/685A/743T, 170L/194V/658V/743T, 170L/423Q, 170L/423Q/451H/476L/557R/658V/659P/743T, 170L/423Q/451H/557R, 170L/423Q/451H/557R/658V/659P/685A/743T, 170L/423Q/451H/557R/659P, 170L/423Q/476L, 170L/423Q/476L/658V/659P, 170L/423Q/476L/658V/659P/660S/685A/743T, 170L/423Q/476L/658V/660S/685A/743T, 170L/423Q/476L/658V/685A, 170L/423Q/476L/658V/685A/743T, 170L/423Q/476L/685A, 170L/423Q/476L/685A/743T, 170L/423Q/557R/658V/659P, 170L/423Q/557R/658V/660S/743T, 170L/423Q/557R/658V/743T, 170L/423Q/557R/659P/660S/685A/743T, 170L/423Q/557R/660S/743T, 170L/423Q/557R/685A, 170L/423Q/557R/685A/743T, 170L/423Q/658V, 170L/423Q/658V/659P/743T, 170L/423Q/658V/660S, 170L/423Q/743T, 170L/451H/557R, 170L/451H/557R/658V/660S, 170L/476L/557R/658V/659P, 170L/476L/557R/658V/685A, 170L/476L/557R/743T, 170L/476L/659P, 170L/557R, 170L/557R/658V/660S/685A/687F, 170L/658V, 170L/658V/659P, 170L/658V/659P/743T, 170L/659P, 170L/660S, 179M, 194V/423Q/451H/557R/658V/659P/743T, 194V/423Q/476L/658V/660S/685A, 194V/423Q/557R/658V/659P/743T, 194V/423Q/685A, 194V/451H/476L/557R/658V/659P, 194V/451H/476L/557R/658V/659P/660S/743T, 194V/557R, 194V/557R/658V/659P/685A, 194V/557R/659P/743T, 194V/658V/659P/685A, 194V/658V/660S, 197K, 198G, 198G/423Q/476L/557R/743T, 198H, 198P, 198V, 205R, 205S, 209P, 209R, 209S, 209V, 209W, 214D, 214R, 2221, 228S, 249E, 250I/269R/331S, 252L, 252L/258H/331S, 252L/258H/331S/352K/467N/506N/674V, 252L/269R, 252L/269R/331S/339E/412A/413K, 252L/269R/331S/352K/412A/674V/704K, 252L/269R/331S/506N/674V, 252L/269R/339E/352K/356G/660P/704K, 252L/269R/339E/352K/412A/699V/704K, 252L/269R/352K/660P, 252L/269R/412A/674V, 252L/269R/413K/467N/660P/699V/704K, 252L/269R/467N/506N, 252L/269R/674V, 252L/331S, 252L/331S/339E/352K/467N/506N, 252L/331S/339E/412A/413K/467N/699V, 252L/331S/352K/412A/413K/467N/660P/704K, 252L/331S/352K/412A/506N/674V, 252L/331S/358N/506N, 252L/331S/412A/413K, 252L/331S/412A/413K/674V, 252L/331S/412A/467N/506N/699V/704K, 252L/331S/412A/506N/674V, 252L/331S/412A/506N/704K, 252L/331S/413K, 252L/331S/467N/660P/674V, 252L/331S/660P, 252L/331S/699V, 252L/339E/352K/467N/674V/699V, 252L/339E/352K/674V/704K, 252L/339E/412A/413K/467N/674V, 252L/339E/412A/413K/674V/699V/704K, 252L/339E/412A/467N/674V, 252L/339E/412A/674V/704K, 252L/339E/467N/506N/699V/704K, 252L/341A/412A/413K, 252L/341A/412A/674V, 252L/352K/412A, 252L/352K/506N, 252L/352K/660P, 252L/358N/674V, 252L/412A, 252L/412A/413K, 252L/412A/467N, 252L/412A/467N/660P/699V/704K, 252L/413K, 252L/413K/506N/660P/704K, 252L/413K/699V, 252L/467N, 252L/467N/506N/674V/704K, 252L/506N/704K, 252L/674V, 252L/699V, 252L/704K, 264A, 264S, 268A/621N, 268G, 268H/510A, 269R, 269R/331S/352K/412A/467N/506N/704K, 269R/331S/352K/467N/506N/699V, 269R/331S/352K/467N/699V, 269R/331S/412A/413K/467N/506N/660P/674V, 269R/331S/413K/506N, 269R/331S/467N/506N/660P, 269R/331S/660P/699V, 269R/331S/674V/699V, 269R/331S/699V, 269R/331S/699V/704K, 269R/339E/506N/704K, 269R/352K/467N/506N/674V, 269R/358N, 269R/358N/412A/660P/704K, 269R/358N/467N/506N/660P/699V, 269R/412A/413K/506N/704K, 269R/412A/413K/674V, 269R/412A/467N/704K, 269R/467N/506N, 269R/506N, 269R/599F/660P/674V/699V, 269R/674V, 269R/699V/704K, 269R/704K, 272S/352K/506N, 275E, 275F, 275L, 275M, 301G, 301M, 301T, 323L, 323Q, 331S, 331S/339E/412A/413K/467N/506N/674V/704K, 331S/339E/412A/467N/506N/704K, 331S/352K/358N/699V/704K, 331S/352K/412A/413K/506N, 331S/358N, 331S/358N/467N/674V/699V/704K, 331S/412A, 331S/412A/413K/467N/699V/704K, 331S/412A/467N/674V/704K, 331S/412A/467N/704K, 331S/412A/660P/699V, 331S/413K/467N/660P/674V, 331S/467N, 331S/467N/506N, 331S/467N/674V/699V, 331S/506N, 331S/506N/674V/699V, 331S/660P/674V/699V, 331S/674V, 331S/674V/699V, 331S/699V, 331S/704K, 339E, 339E/352K, 339E/352K/412A/419K/467N/506N/660P/674V/699V, 339E/352K/412A/467N/660P, 339E/352K/412A/660P/674V, 339E/352K/674V/699V, 339E/412A/413K, 339E/412A/413K/467N/674V/699V, 339E/412A/467N/674V, 339E/467N/674V, 339E/506N, 339E/506N/699V, 339E/506N/704K, 339E/660P, 339E/660P/674V, 341A/352K/704K, 341A/467N, 341A/467N/506N/660P/674V, 341A/467N/674V, 345P, 352G, 352K/358N, 352K/412A/413K/467N/506N/699V, 352K/413K/467N/506N, 352K/413K/674V, 352K/660P, 352K/674V, 355G, 358N/412A, 358N/413K, 358N/674V, 412A, 412A/413K/467N/660P, 412A/413K/467N/674V, 412A/467N/506N/674V/704K, 412A/467N/660P/674V/699V, 412A/660P, 412A/674V, 412A/674V/699V, 413K/467N, 413K/506N, 413K/506N/704K, 413K/674V/699V/704K/740H, 423Q, 423Q/451H/476L/557R/658V/685A, 423Q/451H/476L/658V/685A, 423Q/451H/557R, 423Q/451H/557R/658V, 423Q/451H/557R/658V/685A, 423Q/476L, 423Q/476L/557R/658V/659P/660S, 423Q/476L/557R/658V/659P/685A/743T, 423Q/476L/557R/659P/660S/685A/743T, 423Q/476L/557R/659P/660S/743T, 423Q/476L/658V/660S/743T, 423Q/476L/658V/685A/743T, 423Q/476L/658V/743T, 423Q/557R/658V, 423Q/557R/658V/659P/660S, 423Q/557R/658V/659P/660S/685A, 423Q/557R/658V/660S/743T, 423Q/557R/685A, 423Q/557R/685A/743T, 423Q/658V, 423Q/658V/659P/660S, 423Q/658V/660S, 423Q/660S, 423Q/685A, 423Q/685A/743T, 423Q/743T, 428C, 428G, 428Q, 433G, 433K, 433P/497N, 433R, 433S, 433V, 467N, 467N/506N/699V/704K, 467N/658V/660S/674V/699V, 470E, 470G, 470H, 470V, 476L, 476L/480K/557R/659P, 476L/557R/658V/659P, 476L/557R/658V/660S/685A, 476L/557R/659P, 476L/557R/659P/660S, 476L/658V/659P/660S/685A/743T, 476L/658V/660S/743T, 477E, 477T, 478G, 478I, 478L, 478S, 485A, 485E, 485G, 485I, 485M, 485T, 485V, 489Q/557R/743T, 492M, 492R, 492Y, 501K, 501L, 506N/660P, 506N/674V/699V/704K, 506N/674V/704K, 511L, 514G, 514V, 517A, 517H, 517L, 517M, 517R, 517T, 520V, 525V/557R/659P/660S/743T, 533L, 533P, 534A, 534A/670V, 549V, 557R, 557R/658V, 557R/658V/659P, 557R/658V/659P/660S, 557R/659P/660S, 557R/660S, 557R/743T, 565R, 565S, 565Y, 596S, 598G, 598H, 598K, 598L, 598R, 642E, 658V, 658V/659P, 658V/659P/660S/685A/743T, 658V/659P/685A, 658V/743T, 659P/660S, 659P/660S/685A/743T, 674V, 674V/699V, 685A, 689D, 689N, 695K, 695Q, 695T, 696A, 696G, 696K, 696Q, 699V, 699V/704K, 704K, 707A, 707R, 707S, 725H, 743T, 745P, 747V, 783H, and 821S, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from M12E, M12L, M12S, M12V, P14E, P14V, L15V, E17S, K19A, K19S, F36H, K39E, K39P, K39V, S44A, S44L, R45L, R45S, R82K, Y86G, E87A, E87P, E87R, P95D, P95G, P95R, P122A, P122Q, R139D/T395A, R139S, P159Q, E167D, R170L/A194V, R170L/A194V/A423Q/A451H/P557R/L658V/A660S/N743T, R170L/A194V/A423Q/M476L/N743T, R170L/A194V/A423Q/P557R/L658V/S659P/N743T, R170L/A194V/A423Q/P557R/L658V/A660S/H685A, R170L/A194V/A423Q/P557R/L658V/N743T, R170L/A194V/A451H/M476L/P557R/L658V, R170L/A194V/A451H/M476L/P557R/L658V/S659P/A660S/N743T, R170L/A194V/A451H/M476L/P557R/L658V/H685A, R170L/A194V/A451H/P557R/S659P/A660S, R170L/A194V/M476L/P557R/S659P, R170L/A194V/P557R, R170L/A194V/L658V, R170L/A194V/L658V/S659P/A660S/H685A/N743T, R170L/A194V/L658V/N743T, R170L/A423Q, R170L/A423Q/A451H/M476L/P557R/L658V/S659P/N743T, R170L/A423Q/A451H/P557R, R170L/A423Q/A451H/P557R/L658V/S659P/H685A/N743T, R170L/A423Q/A451H/P557R/S659P, R170L/A423Q/M476L, R170L/A423Q/M476L/L658V/S659P, R170L/A423Q/M476L/L658V/S659P/A660S/H685A/N743T, R170L/A423Q/M476L/L658V/A660S/H685A/N743T, R170L/A423Q/M476L/L658V/H685A, R170L/A423Q/M476L/L658V/H685A/N743T, R170L/A423Q/M476L/H685A, R170L/A423Q/M476L/H685A/N743T, R170L/A423Q/P557R/L658V/S659P, R170L/A423Q/P557R/L658V/A660S/N743T, R170L/A423Q/P557R/L658V/N743T, R170L/A423Q/P557R/S659P/A660S/H685A/N743T, R170L/A423Q/P557R/A660S/N743T, R170L/A423Q/P557R/H685A, R170L/A423Q/P557R/H685A/N743T, R170L/A423Q/L658V, R170L/A423Q/L658V/S659P/N743T, R170L/A423Q/L658V/A660S, R170L/A423Q/N743T, R170L/A451H/P557R, R170L/A451H/P557R/L658V/A660S, R170L/M476L/P557R/L658V/S659P, R170L/M476L/P557R/L658V/H685A, R170L/M476L/P557R/N743T, R170L/M476L/S659P, R170L/P557R, R170L/P557R/L658V/A660S/H685A/L687F, R170L/L658V, R170L/L658V/S659P, R170L/L658V/S659P/N743T, R170L/S659P, R170L/A660S, K179M, A194V/A423Q/A451H/P557R/L658V/S659P/N743T, A194V/A423Q/M476L/L658V/A660S/H685A, A194V/A423Q/P557R/L658V/S659P/N743T, A194V/A423Q/H685A, A194V/A451H/M476L/P557R/L658V/S659P, A194V/A451H/M476L/P557R/L658V/S659P/A660S/N743T, A194V/P557R, A194V/P557R/L658V/S659P/H685A, A194V/P557R/S659P/N743T, A194V/L658V/S659P/H685A, A194V/L658V/A660S, P197K, S198G, S198G/A423Q/M476L/P557R/N743T, S198H, S198P, S198V, K205R, K205S, E209P, E209R, E209S, E209V, E209W, K214D, K214R, L222I, N228S, K249E, L250I/G269R/L331S, R252L, R252L/R258H/L331S, R252L/R258H/L331S/R352K/A467N/E506N/1674V, R252L/G269R, R252L/G269R/L331S/G339E/G412A/E413K, R252L/G269R/L331S/R352K/G412A/1674V/R704K, R252L/G269R/L331S/E506N/1674V, R252L/G269R/G339E/R352K/E356G/A660P/R704K, R252L/G269R/G339E/R352K/G412A/A699V/R704K, R252L/G269R/R352K/A660P, R252L/G269R/G412A/1674V, R252L/G269R/E413K/A467N/A660P/A699V/R704K, R252L/G269R/A467N/E506N, R252L/G269R/1674V, R252L/L331S, R252L/L331S/G339E/R352K/A467N/E506N, R252L/L331S/G339E/G412A/E413K/A467N/A699V, R252L/L331S/R352K/G412A/E413K/A467N/A660P/R704K, R252L/L331S/R352K/G412A/E506N/1674V, R252L/L331S/R358N/E506N, R252L/L331S/G412A/E413K, R252L/L331S/G412A/E413K/1674V, R252L/L331S/G412A/A467N/E506N/A699V/R704K, R252L/L331S/G412A/E506N/1674V, R252L/L331S/G412A/E506N/R704K, R252L/L331S/E413K, R252L/L331S/A467N/A660P/1674V, R252L/L331S/A660P, R252L/L331S/A699V, R252L/G339E/R352K/A467N/1674V/A699V, R252L/G339E/R352K/1674V/R704K, R252L/G339E/G412A/E413K/A467N/1674V, R252L/G339E/G412A/E413K/1674V/A699V/R704K, R252L/G339E/G412A/A467N/1674V, R252L/G339E/G412A/1674V/R704K, R252L/G339E/A467N/E506N/A699V/R704K, R252L/V341A/G412A/E413K, R252L/V341A/G412A/1674V, R252L/R352K/G412A, R252L/R352K/E506N, R252L/R352K/A660P, R252L/R358N/1674V, R252L/G412A, R252L/G412A/E413K, R252L/G412A/A467N, R252L/G412A/A467N/A660P/A699V/R704K, R252L/E413K, R252L/E413K/E506N/A660P/R704K, R252L/E413K/A699V, R252L/A467N, R252L/A467N/E506N/1674V/R704K, R252L/E506N/R704K, R252L/1674V, R252L/A699V, R252L/R704K, E264A, E264S, R268A/S621N, R268G, R268H/P510A, G269R, G269R/L331S/R352K/G412A/A467N/E506N/R704K, G269R/L331S/R352K/A467N/E506N/A699V, G269R/L331S/R352K/A467N/A699V, G269R/L331S/G412A/E413K/A467N/E506N/A660P/1674V, G269R/L331S/E413K/E506N, G269R/L331S/A467N/E506N/A660P, G269R/L331S/A660P/A699V, G269R/L331S/1674V/A699V, G269R/L331S/A699V, G269R/L331S/A699V/R704K, G269R/G339E/E506N/R704K, G269R/R352K/A467N/E506N/1674V, G269R/R358N, G269R/R358N/G412A/A660P/R704K, G269R/R358N/A467N/E506N/A660P/A699V, G269R/G412A/E413K/E506N/R704K, G269R/G412A/E413K/1674V, G269R/G412A/A467N/R704K, G269R/A467N/E506N, G269R/E506N, G269R/L599F/A660P/1674V/A699V, G269R/1674V, G269R/A699V/R704K, G269R/R704K, E272S/R352K/E506N, W275E, W275F, W275L, W275M, L301G, L301M, L301T, P323L, P323Q, L331S, L331S/G339E/G412A/E413K/A467N/E506N/1674V/R704K, L331S/G339E/G412A/A467N/E506N/R704K, L331S/R352K/R358N/A699V/R704K, L331S/R352K/G412A/E413K/E506N, L331S/R358N, L331S/R358N/A467N/1674V/A699V/R704K, L331S/G412A, L331S/G412A/E413K/A467N/A699V/R704K, L331S/G412A/A467N/1674V/R704K, L331S/G412A/A467N/R704K, L331S/G412A/A660P/A699V, L331S/E413K/A467N/A660P/1674V, L331S/A467N, L331S/A467N/E506N, L331S/A467N/1674V/A699V, L331S/E506N, L331S/E506N/1674V/A699V, L331S/A660P/1674V/A699V, L331S/1674V, L331S/1674V/A699V, L331S/A699V, L331S/R704K, G339E, G339E/R352K, G339E/R352K/G412A/E419K/A467N/E506N/A660P/1674V/A699V, G339E/R352K/G412A/A467N/A660P, G339E/R352K/G412A/A660P/1674V, G339E/R352K/1674V/A699V, G339E/G412A/E413K, G339E/G412A/E413K/A467N/1674V/A699V, G339E/G412A/A467N/1674V, G339E/A467N/1674V, G339E/E506N, G339E/E506N/A699V, G339E/E506N/R704K, G339E/A660P, G339E/A660P/1674V, V341A/R352K/R704K, V341A/A467N, V341A/A467N/E506N/A660P/1674V, V341A/A467N/1674V, E345P, R352G, R352K/R358N, R352K/G412A/E413K/A467N/E506N/A699V, R352K/E413K/A467N/E506N, R352K/E413K/1674V, R352K/A660P, R352K/1674V, K355G, R358N/G412A, R358N/E413K, R358N/1674V, G412A, G412A/E413K/A467N/A660P, G412A/E413K/A467N/1674V, G412A/A467N/E506N/1674V/R704K, G412A/A467N/A660P/1674V/A699V, G412A/A660P, G412A/1674V, G412A/1674V/A699V, E413K/A467N, E413K/E506N, E413K/E506N/R704K, E413K/I674V/A699V/R704K/P740H, A423Q, A423Q/A451H/M476L/P557R/L658V/H685A, A423Q/A451H/M476L/L658V/H685A, A423Q/A451H/P557R, A423Q/A451H/P557R/L658V, A423Q/A451H/P557R/L658V/H685A, A423Q/M476L, A423Q/M476L/P557R/L658V/S659P/A660S, A423Q/M476L/P557R/L658V/S659P/H685A/N743T, A423Q/M476L/P557R/S659P/A660S/H685A/N743T, A423Q/M476L/P557R/S659P/A660S/N743T, A423Q/M476L/L658V/A660S/N743T, A423Q/M476L/L658V/H685A/N743T, A423Q/M476L/L658V/N743T, A423Q/P557R/L658V, A423Q/P557R/L658V/S659P/A660S, A423Q/P557R/L658V/S659P/A660S/H685A, A423Q/P557R/L658V/A660S/N743T, A423Q/P557R/H685A, A423Q/P557R/H685A/N743T, A423Q/L658V, A423Q/L658V/S659P/A660S, A423Q/L658V/A660S, A423Q/A660S, A423Q/H685A, A423Q/H685A/N743T, A423Q/N743T, R428C, R428G, R428Q, E433G, E433K, E433P/D497N, E433R, E433S, E433V, A467N, A467N/E506N/A699V/R704K, A467N/L658V/A660S/1674V/A699V, L470E, L470G, L470H, L470V, M476L, M476L/E480K/P557R/S659P, M476L/P557R/L658V/S659P, M476L/P557R/L658V/A660S/H685A, M476L/P557R/S659P, M476L/P557R/S659P/A660S, M476L/L658V/S659P/A660S/H685A/N743T, M476L/L658V/A660S/N743T, R477E, R477T, R478G, R478I, R478L, R478S, R485A, R485E, R485G, R485I, R485M, R485T, R485V, H489Q/P557R/N743T, N492M, N492R, N492Y, R501K, R501L, E506N/A660P, E506N/1674V/A699V/R704K, E506N/1674V/R704K, P511L, K514G, K514V, K517A, K517H, K517L, K517M, K517R, K517T, K520V, A525V/P557R/S659P/A660S/N743T, E533L, E533P, V534A, V534A/A670V, K549V, P557R, P557R/L658V, P557R/L658V/S659P, P557R/L658V/S659P/A660S, P557R/S659P/A660S, P557R/A660S, P557R/N743T, K565R, K565S, K565Y, R596S, P598G, P598H, P598K, P598L, P598R, Q642E, L658V, L658V/S659P, L658V/S659P/A660S/H685A/N743T, L658V/S659P/H685A, L658V/N743T, S659P/A660S, S659P/A660S/H685A/N743T, I674V, 1674V/A699V, H685A, Q689D, Q689N, Y695K, Y695Q, Y695T, E696A, E696G, E696K, E696Q, A699V, A699V/R704K, R704K, Q707A, Q707R, Q707S, T725H, N743T, R745P, K747V, E783H, and P821S, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from 12, 13, 14, 15, 17, 19/214, 45, 84, 87, 95, 122, 159, 170/194/476/557/659, 170/423/476/658/685, 170/423/557/658/743, 170/476/557/658/659, 170/476/557/743, 178, 194/658/660, 198, 209, 214, 222, 228, 249, 252, 252/258/331, 252/269, 252/269/412/674, 252/269/674, 252/331, 252/331/352/412/506/674, 252/331/467/660/674, 252/331/660, 252/331/699, 252/352/412, 252/412, 252/412/467/660/699/704, 252/413, 252/467/506/674/704, 252/704, 264, 268, 268/621, 269/331/699, 269/412/467/704, 269/467/506, 269/674, 269/695, 269/699/704, 275, 276, 301, 323, 331/358, 331/412/660/699, 331/674, 331/704, 345, 352/674, 412/467/660/674/699, 412/660, 412/674, 430, 430/837, 433, 467, 470, 476/557/658/659, 476/658/660/743, 477, 478, 485, 492, 511, 514, 517, 520, 533, 534, 534/670, 549, 557/658/659, 565, 596, 598, 642, 658/659, 674, 689, 695, 696, 699/704, 707, 725, and 783, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from 12E, 12V, 13G, 14E, 14V, 15V, 17G, 17K, 17S, 19L/214R, 45S, 84N, 84T, 87A, 87R, 87S, 95D, 95G, 122Q, 159Q, 170L/194V/476L/557R/659P, 170L/423Q/476L/658V/685A, 170L/423Q/557R/658V/743T, 170L/476L/557R/658V/659P, 170L/476L/557R/743T, 178G, 178L, 178N, 178S, 194V/658V/660S, 198V, 209S, 209W, 214R, 2221, 228D, 228S, 249R, 252L, 252L/258H/331S, 252L/269R, 252L/269R/412A/674V, 252L/269R/674V, 252L/331S, 252L/331S/352K/412A/506N/674V, 252L/331S/467N/660P/674V, 252L/331S/660P, 252L/331S/699V, 252L/352K/412A, 252L/412A, 252L/412A/467N/660P/699V/704K, 252L/413K, 252L/467N/506N/674V/704K, 252L/704K, 264T, 268A/621N, 268G, 269C/695R, 269R/331S/699V, 269R/412A/467N/704K, 269R/467N/506N, 269R/674V, 269R/699V/704K, 275E, 275G, 275M, 275V, 276A, 276D, 276G, 276M, 301G, 323L, 323Q, 323S, 331S/358N, 331S/412A/660P/699V, 331S/674V, 331S/704K, 345G, 345P, 352K/674V, 412A/467N/660P/674V/699V, 412A/660P, 412A/674V, 430A/837S, 430F, 433K, 433R, 433S, 433V, 467N, 470M, 470R, 476L/557R/658V/659P, 476L/658V/660S/743T, 477E, 478G, 478L, 478M, 478S, 485A, 485G, 485I, 485T, 485W, 492R, 492Y, 511C, 511L, 514G, 514V, 517A, 517H, 517L, 517R, 517T, 520V, 533L, 533P, 534A, 534A/670V, 549V, 557R/658V/659P, 565R, 596S, 598G, 598K, 598L, 598R, 642E, 642R, 658V/659P, 674V, 689D, 695K, 695S, 695T, 696G, 696K, 696T, 699V/704K, 707A, 725H, and 783H, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from M12E, M12V, L13G, P14E, P14V, L15V, E17G, E17K, E17S, K19L/K214R, R45S, E84N, E84T, E87A, E87R, E87S, P95D, P95G, P122Q, P159Q, R170L/A194V/M476L/P557R/S659P, R170L/A423Q/M476L/L658V/H685A, R170L/A423Q/P557R/L658V/N743T, R170L/M476L/P557R/L658V/S659P, R170L/M476L/P557R/N743T, E178G, E178L, E178N, E178S, A194V/L658V/A660S, S198V, E209S, E209W, K214R, L222I, N228D, N228S, K249R, R252L, R252L/R258H/L331S, R252L/G269R, R252L/G269R/G412A/1674V, R252L/G269R/1674V, R252L/L331S, R252L/L331S/R352K/G412A/E506N/1674V, R252L/L331S/A467N/A660P/1674V, R252L/L331S/A660P, R252L/L331S/A699V, R252L/R352K/G412A, R252L/G412A, R252L/G412A/A467N/A660P/A699V/R704K, R252L/E413K, R252L/A467N/E506N/1674V/R704K, R252L/R704K, E264T, R268A/S621N, R268G, G269C/Y695R, G269R/L331S/A699V, G269R/G412A/A467N/R704K, G269R/A467N/E506N, G269R/1674V, G269R/A699V/R704K, W275E, W275G, W275M, W275V, E276A, E276D, E276G, E276M, L301G, P323L, P323Q, P323S, L331S/R358N, L331S/G412A/A660P/A699V, L331S/1674V, L331S/R704K, E345G, E345P, R352K/1674V, G412A/A467N/A660P/1674V/A699V, G412A/A660P, G412A/1674V, Q430A/L837S, Q430F, E433K, E433R, E433S, E433V, A467N, L470M, L470R, M476L/P557R/L658V/S659P, M476L/L658V/A660S/N743T, R477E, R478G, R478L, R478M, R478S, R485A, R485G, R485I, R485T, R485W, N492R, N492Y, P511C, P511L, K514G, K514V, K517A, K517H, K517L, K517R, K517T, K520V, E533L, E533P, V534A, V534A/A670V, K549V, P557R/L658V/S659P, K565R, R596S, P598G, P598K, P598L, P598R, Q642E, Q642R, L658V/S659P, 1674V, Q689D, Y695K, Y695S, Y695T, E696G, E696K, E696T, A699V/R704K, Q707A, T725H, and E783H, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from 12, 13, 14, 15, 17, 84, 87, 122, 159, 170/423/476/658/685, 170/476/557/743, 178, 198, 205, 209, 222, 228, 249, 252, 252/269, 252/269/412/674, 252/269/674, 252/331, 252/331/352/412/506/674, 252/331/467/660/674, 252/331/699, 252/412, 252/412/467/660/699/704, 252/413, 252/467/506/674/704, 252/704, 264, 269/331/699, 269/412/467/704, 269/467/506, 269/674, 275, 276, 301, 322, 323, 331/358, 331/412/660/699, 331/674, 331/704, 345, 352/674, 355, 412/674, 430, 430/837, 433, 467, 470, 476/658/660/743, 477, 478, 485, 492, 511, 514, 517, 520, 533, 534, 534/670, 549, 557/658/659, 565, 596, 598, 642, 674, 689, 695, 696, 699/704, 703, 725, and 821, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from 12S, 12V, 13G, 14V, 15V, 17G, 17K, 17S, 84N, 84T, 87A, 87Q, 87R, 87S, 122Q, 159Q, 170L/423Q/476L/658V/685A, 170L/476L/557R/743T, 178G, 178L, 178N, 178S, 198V, 205R, 209S, 2221, 228D, 228S, 249G, 249R, 252L, 252L/269R, 252L/269R/412A/674V, 252L/269R/674V, 252L/331S, 252L/331S/352K/412A/506N/674V, 252L/331S/467N/660P/674V, 252L/331S/699V, 252L/412A, 252L/412A/467N/660P/699V/704K, 252L/413K, 252L/467N/506N/674V/704K, 252L/704K, 264S, 264T, 269R/331S/699V, 269R/412A/467N/704K, 269R/467N/506N, 269R/674V, 275E, 275G, 275V, 276A, 276D, 276G, 276M, 301G, 322L, 323L, 331S/358N, 331S/412A/660P/699V, 331S/674V, 331S/704K, 345G, 352K/674V, 355F, 412A/674V, 430A/837S, 430F, 433R, 433S, 433V, 467N, 470E, 470M, 470R, 476L/658V/660S/743T, 477E, 478G, 478M, 485A, 485E, 485G, 485I, 485W, 492R, 492Y, 511C, 511E, 511L, 514G, 514V, 517A, 517H, 517T, 520V, 533L, 533P, 534A, 534A/670V, 549V, 557R/658V/659P, 565R, 565Y, 596S, 598G, 598L, 642R, 674V, 689D, 695K, 695S, 695T, 696G, 696K, 696T, 696W, 699V/704K, 703K, 725L, and 821R, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from M12S, M12V, L13G, P14V, L15V, E17G, E17K, E17S, E84N, E84T, E87A, E87Q, E87R, E87S, P122Q, P159Q, R170L/A423Q/M476L/L658V/H685A, R170L/M476L/P557R/N743T, E178G, E178L, E178N, E178S, S198V, K205R, E209S, L222I, N228D, N228S, K249G, K249R, R252L, R252L/G269R, R252L/G269R/G412A/1674V, R252L/G269R/1674V, R252L/L331S, R252L/L331S/R352K/G412A/E506N/1674V, R252L/L331S/A467N/A660P/1674V, R252L/L331S/A699V, R252L/G412A, R252L/G412A/A467N/A660P/A699V/R704K, R252L/E413K, R252L/A467N/E506N/1674V/R704K, R252L/R704K, E264S, E264T, G269R/L331S/A699V, G269R/G412A/A467N/R704K, G269R/A467N/E506N, G269R/1674V, W275E, W275G, W275V, E276A, E276D, E276G, E276M, L301G, R322L, P323L, L331S/R358N, L331S/G412A/A660P/A699V, L331S/1674V, L331S/R704K, E345G, R352K/1674V, K355F, G412A/1674V, Q430A/L837S, Q430F, E433R, E433S, E433V, A467N, L470E, L470M, L470R, M476L/L658V/A660S/N743T, R477E, R478G, R478M, R485A, R485E, R485G, R485I, R485W, N492R, N492Y, P511C, P511E, P511L, K514G, K514V, K517A, K517H, K517T, K520V, E533L, E533P, V534A, V534A/A670V, K549V, P557R/L658V/S659P, K565R, K565Y, R596S, P598G, P598L, Q642R, 1674V, Q689D, Y695K, Y695S, Y695T, E696G, E696K, E696T, E696W, A699V/R704K, Q703K, T725L, and P821R, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from 12, 15, 17, 252, 252/331, 252/331/352/412/506/674, 252/413, 252/704, 269/412/467/704, 269/467/506, 269/674, 301, 331/704, 345, 352/674, 355, 430/837, 492, 511, 514, 520, 598, 642, 674, 695, 696, 699/704, and 703, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from 12V, 15V, 17G, 17K, 252L, 252L/331S, 252L/331S/352K/412A/506N/674V, 252L/413K, 252L/704K, 269R/412A/467N/704K, 269R/467N/506N, 269R/674V, 301G, 331S/704K, 345G, 352K/674V, 355F, 430A/837S, 492R, 492Y, 511C, 511L, 514G, 514V, 520V, 598G, 642R, 674V, 695S, 695T, 696T, 699V/704K, and 703K, or combinations thereof, wherein the amino acid positions are relative to the reference sequence of SEQ ID NO: 178.

In some embodiments, the present invention provides an engineered polynucleotide encoding an engineered DNA polymerase comprising one or more substitutions or substitution sets selected from M12V, L15V, E17G, E17K, R252L, R252L/L331S, R252L/L331S/R352K/G412A/E506N/1674V, R252L/E413K, R252L/R704K, G269R/G412A/A467N/R704K, G269R/A467N/E506N, G269R/1674V, L301G, L331S/R704K, E345G, R352K/1674V, K355F, Q430A/L837S, N492R, N492Y, P511C, P511L, K514G, K514V, K520V, P598G, Q642R, 1674V, Y695S, Y695T, E696T, A699V/R704K, and Q703K, or combinations thereof, wherein the amino acid positions are relative to the reference sequence of SEQ ID NO: 178.

The present invention also provides engineered polynucleotides encoding engineered DNA polymerases or functional fragments thereof, comprising a polypeptide sequences having at least 75% or more sequence identity to a reference sequence comprising residues 12 to 841 of SEQ ID NO: 994, or to a reference sequence of SEQ ID NO: 994, wherein the polypeptide sequences comprise one or more substitutions relative to the reference sequence comprising residues 12 to 841 of SEQ ID NO: 994, or to the reference sequence of SEQ ID NO: 994.

In some embodiments, the engineered polynucleotide encodes an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 994 or to the reference sequence of SEQ ID NO: 994.

In some embodiments, the present invention provides an engineered polynucleotide encoding an engineered DNA polymerase comprising one or more substitutions or substitution sets selected from 2, 5, 7, 8, 12/15/269/355/412/413/492/704, 12/252/413/492, 32, 33, 35/304, 38, 40, 65, 80, 81, 140, 151, 169, 194, 194/252, 194/252/358, 194/252/358/412/467, 194/252/358/412/467/658/696/743, 194/252/412/696, 194/252/467/696/743, 194/252/743, 194/358, 194/358/412, 194/358/658, 194/358/704, 194/412, 194/412/467, 194/412/467/743, 194/467/511/658, 194/467/658, 194/467/704, 194/467/743, 194/743, 218, 226, 242, 252, 252/412/658, 252/467/696, 258/347, 279, 284, 289, 298, 300, 304, 311, 329, 331, 340, 358, 375, 375/376, 406, 420, 434, 440, 481, 489, 490, 518, 529, 543, 560, 592, 635, 639, 645, 658, 658/704, 666, 685, 694, 704, 713, 717, 718, 721, 740, 746, 782, 804, 807, 814, and 829, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from 2G, 2R, 5A, 5G, 5V, 5W, 7Q, 8R, 12V/15V/269R/355F/412A/413K/492R/704K, 12V/252L/413K/492R, 32L, 33K, 35L/304G, 38E, 38G, 38R, 38S, 38V, 40N, 40R, 40S, 65S, 80G, 81A, 140M, 151K, 169S, 194V, 194V/252L, 194V/252L/358N, 194V/252L/358N/412A/467N, 194V/252L/358N/412A/467N/658V/696T/743T, 194V/252L/412A/696T, 194V/252L/467N/696T/743T, 194V/252L/743T, 194V/358N, 194V/358N/412A, 194V/358N/658V, 194V/358N/704K, 194V/412A, 194V/412A/467N, 194V/412A/467N/743T, 194V/467N/511C/658V, 194V/467N/658V, 194V/467N/704K, 194V/467N/743T, 194V/743T, 218T, 218Y, 226E, 242G, 242N, 252L, 252L/412A/658V, 252L/467N/696T, 258L/347H, 279R, 284A, 289D, 289G, 298A, 298S, 300A, 300F, 300L, 300N, 300R, 300T, 300W, 304C, 304G, 304T, 304V, 311C, 329L, 329S, 331D, 331Q, 331Y, 340T, 358N, 375A/376I, 3751, 406M, 420S, 420T, 434K, 440N, 440T, 481D, 481G, 489R, 490R, 490V, 518L, 518R, 529R, 529T, 529V, 543A, 560G, 560K, 592A, 592S, 635I, 639Q, 645L, 645M, 645R, 658A, 658C, 658E, 658R, 658V/704K, 666D, 666G, 666K, 666M, 666N, 666R, 666S, 666T, 666V, 685T, 694S, 694T, 704Q, 704V, 713V, 717T, 718R, 718S, 721V, 721W, 740G, 746G, 746Q, 746R, 782S, 804D, 804Q, 807H, 814A, 814I, 814R, 829L, and 829R, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from H2G, H2R, H5A, H5G, H5V, H5W, H7Q, G8R, M12V/L15V/G269R/K355F/G412A/E413K/Y492R/R704K, M12V/R252L/E413K/Y492R, Y32L, R33K, F35L/E304G, L38E, L38G, L38R, L38S, L38V, G40N, G40R, G40S, E65S, S80G, F81A, E140M, R151K, H169S, A194V, A194V/R252L, A194V/R252L/R358N, A194V/R252L/R358N/G412A/A467N, A194V/R252L/R358N/G412A/A467N/L658V/E696T/N743T, A194V/R252L/G412A/E696T, A194V/R252L/A467N/E696T/N743T, A194V/R252L/N743T, A194V/R358N, A194V/R358N/G412A, A194V/R358N/L658V, A194V/R358N/R704K, A194V/G412A, A194V/G412A/A467N, A194V/G412A/A467N/N743T, A194V/A467N/P511C/L658V, A194V/A467N/L658V, A194V/A467N/R704K, A194V/A467N/N743T, A194V/N743T, E218T, E218Y, L226E, L242G, L242N, R252L, R252L/G412A/L658V, R252L/A467N/E696T, R258L/P347H, K279R, R284A, S289D, S289G, E298A, E298S, P300A, P300F, P300L, P300N, P300R, P300T, P300W, E304C, E304G, E304T, E304V, P311C, E329L, E329S, L331D, L331Q, L331Y, R340T, R358N, D375A/L376I, D375I, E406M, R420S, R420T, R434K, E440N, E440T, E481D, E481G, H489R, S490R, S490V, T518L, T518R, E529R, E529T, E529V, Q543A, A560G, A560K, N592A, N592S, E635I, R639Q, K645L, K645M, K645R, L658A, L658C, L658E, L658R, L658V/R704K, L666D, L666G, L666K, L666M, L666N, L666R, L666S, L666T, L666V, H685T, P694S, P694T, R704Q, R704V, K713V, E717T, K718R, K718S, E721V, E721W, P740G, V746G, V746Q, V746R, R782S, R804D, R804Q, E807H, E814A, E814I, E814R, E829L, and E829, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from 2, 5, 8, 12/15/269/355/412/413/492/704, 12/252/413/492, 38, 40, 65, 80, 140, 151, 218, 226, 230, 242, 258/347, 279, 284, 289, 298, 300, 304, 309, 311, 329, 331, 340, 375, 375/376, 406, 420, 434, 441, 489, 490, 518, 529, 543, 560, 592, 635, 639, 645, 658, 666, 685, 704, 713, 717, 718, 721, 740, 743, 746, 782, 804, 807, 814, and 829, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from 2G, 2V, 5A, 5G, 5L, 5V, 5W, 8R, 12V/15V/269R/355F/412A/413K/492R/704K, 12V/252L/413K/492R, 38G, 38R, 38S, 38V, 40R, 40S, 65G, 65H, 65S, 80G, 80M, 140M, 151K, 218Y, 226E, 230G, 242A, 242G, 242R, 258L/347H, 279R, 284A, 284G, 289D, 298A, 298S, 300F, 300N, 300R, 304C, 304G, 304R, 304V, 309G, 311C, 311G, 329L, 331D, 331G, 331Y, 340T, 375A/376I, 375E, 375L, 406G, 420S, 420T, 434K, 441R, 489R, 490R, 490V, 518M, 518R, 529V, 543A, 543L, 543R, 560G, 592A, 592S, 635I, 639Q, 645R, 658Q, 658R, 666G, 666Q, 666R, 666V, 685F, 704V, 713V, 717T, 718R, 721D, 721M, 721R, 721W, 740L, 740S, 743S, 746Q, 782A, 804G, 807G, 807H, 8141, 814M, 814R, 829L, and 829R, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from H2G, H2V, H5A, H5G, H5L, H5V, H5W, G8R, M12V/L15V/G269R/K355F/G412A/E413K/Y492R/R704K, M12V/R252L/E413K/Y492R, L38G, L38R, L38S, L38V, G40R, G40S, E65G, E65H, E65S, S80G, S80M, E140M, R151K, E218Y, L226E, D230G, L242A, L242G, L242R, R258L/P347H, K279R, R284A, R284G, S289D, E298A, E298S, P300F, P300N, P300R, E304C, E304G, E304R, E304V, P309G, P311C, P311G, E329L, L331D, L331G, L331Y, R340T, D375A/L376I, D375E, D375L, E406G, R420S, R420T, R434K, E441R, H489R, S490R, S490V, T518M, T518R, E529V, Q543A, Q543L, Q543R, A560G, N592A, N592S, E635I, R639Q, K645R, L658Q, L658R, L666G, L666Q, L666R, L666V, H685F, R704V, K713V, E717T, K718R, E721D, E721M, E721R, E721W, P740L, P740S, N743S, V746Q, R782A, R804G, E807G, E807H, E814I, E814M, E814R, E829L, and E829R, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from 2, 5, 8, 12/15/269/355/412/413/492/704, 12/252/413/492, 32, 38, 40, 65, 98, 140, 151, 169, 202, 218, 226, 227, 230, 242, 258/347, 279, 284, 298, 300, 304, 309, 311, 329, 331, 340, 375, 375/376, 406, 420, 434, 440, 441, 481, 489, 490, 518, 529, 543, 560, 592, 635, 639, 645, 658, 666, 685, 692, 694, 704, 713, 717, 718, 721, 740, 746, 782, 804, 807, 814, and 829, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from 2G, 2P, 2V, 5L, 5V, 8R, 12V/15V/269R/355F/412A/413K/492R/704K, 12V/252L/413K/492R, 32L, 38V, 40N, 40S, 65G, 65H, 65S, 98G, 98N, 140M, 151K, 169S, 202Q, 218G, 218Y, 226E, 227Y, 230G, 242A, 242G, 242R, 258L/347H, 279R, 284G, 298A, 298S, 300A, 300F, 300T, 304C, 304G, 304V, 309G, 311G, 329L, 329S, 331G, 331Q, 331V, 331Y, 340S, 340T, 375A, 375A/376I, 375E, 375L, 375M, 375R, 406M, 406Q, 420S, 420T, 434K, 440S, 440T, 441G, 481D, 481G, 489R, 490R, 490V, 518L, 518M, 529L, 529T, 529V, 543A, 543L, 543R, 560G, 560K, 592A, 592S, 635G, 635I, 635T, 639Q, 645F, 645L, 645M, 645N, 645R, 658E, 658Q, 658R, 658S, 666D, 666E, 666G, 666K, 666N, 666Q, 666R, 666S, 666V, 685F, 692S, 694S, 704Q, 704V, 713S, 713V, 717T, 717Y, 718S, 721D, 721L, 721M, 721R, 721V, 721W, 740L, 740S, 746G, 746Q, 746R, 782A, 782S, 804A, 804D, 807G, 814A, 814M, 814R, 829L, and 829R, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from H2G, H2P, H2V, H5L, H5V, G8R, M12V/L15V/G269R/K355F/G412A/E413K/Y492R/R704K, M12V/R252L/E413K/Y492R, Y32L, L38V, G40N, G40S, E65G, E65H, E65S, E98G, E98N, E140M, R151K, H169S, P202Q, E218G, E218Y, L226E, K227Y, D230G, L242A, L242G, L242R, R258L/P347H, K279R, R284G, E298A, E298S, P300A, P300F, P300T, E304C, E304G, E304V, P309G, P311G, E329L, E329S, L331G, L331Q, L331V, L331Y, R340S, R340T, D375A, D375A/L376I, D375E, D375L, D375M, D375R, E406M, E406Q, R420S, R420T, R434K, E440S, E440T, E441G, E481D, E481G, H489R, S490R, S490V, T518L, T518M, E529L, E529T, E529V, Q543A, Q543L, Q543R, A560G, A560K, N592A, N592S, E635G, E635I, E635T, R639Q, K645F, K645L, K645M, K645N, K645R, L658E, L658Q, L658R, L658S, L666D, L666E, L666G, L666K, L666N, L666Q, L666R, L666S, L666V, H685F, G692S, P694S, R704Q, R704V, K713S, K713V, E717T, E717Y, K718S, E721D, E721L, E721M, E721R, E721V, E721W, P740L, P740S, V746G, V746Q, V746R, R782A, R782S, R804A, R804D, E807G, E814A, E814M, E814R, E829L, and E829R, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from 12/15/269/355/412/413/492/704, 12/252/413/492, 194, 194/252, 194/252/358/412/467, 194/252/358/412/467/658/696/743, 194/252/412/696, 194/252/467/658, 194/252/467/696/743, 194/252/467/704, 194/252/696/704, 194/252/704/743, 194/252/743, 194/358, 194/358/412, 194/358/467/658/696/704, 194/358/658, 194/412, 194/412/467, 194/412/658, 194/467, 194/467/658, 194/467/658/743, 194/467/704, 194/467/743, 194/658, 194/743, 242, 252/358/696/704, 252/412/658, 252/467/696, 258/347, 284, 304, 331, 434, 489, 543, 560, 592, 658, 666, 685, 717, 721, and 814, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from 12V/15V/269R/355F/412A/413K/492R/704K, 12V/252L/413K/492R, 194V, 194V/252L, 194V/252L/358N/412A/467N, 194V/252L/358N/412A/467N/658V/696T/743T, 194V/252L/412A/696T, 194V/252L/467N/658V, 194V/252L/467N/696T/743T, 194V/252L/467N/704K, 194V/252L/696T/704K, 194V/252L/704K/743T, 194V/252L/743T, 194V/358N, 194V/358N/412A, 194V/358N/467N/658V/696T/704K, 194V/358N/658V, 194V/412A, 194V/412A/467N, 194V/412A/658V, 194V/467N, 194V/467N/658V, 194V/467N/658V/743T, 194V/467N/704K, 194V/467N/743T, 194V/658V, 194V/743T, 242G, 252L/358N/696T/704K, 252L/412A/658V, 252L/467N/696T, 258L/347H, 284A, 304G, 304V, 331G, 434K, 489R, 543A, 560G, 592A, 658E, 666R, 685F, 717T, 721M, 721R, and 814M, or combinations thereof, wherein the amino acid positions are relative to the reference sequence of SEQ ID NO: 994.

In some embodiments, the present invention provides an engineered polynucleotide encoding an engineered DNA polymerase comprising one or more substitutions or substitution sets selected from M12V/L15V/G269R/K355F/G412A/E413K/Y492R/R704K, M12V/R252L/E413K/Y492R, A194V, A194V/R252L, A194V/R252L/R358N/G412A/A467N, A194V/R252L/R358N/G412A/A467N/L658V/E696T/N743T, A194V/R252L/G412A/E696T, A194V/R252L/A467N/L658V, A194V/R252L/A467N/E696T/N743T, A194V/R252L/A467N/R704K, A194V/R252L/E696T/R704K, A194V/R252L/R704K/N743T, A194V/R252L/N743T, A194V/R358N, A194V/R358N/G412A, A194V/R358N/A467N/L658V/E696T/R704K, A194V/R358N/L658V, A194V/G412A, A194V/G412A/A467N, A194V/G412A/L658V, A194V/A467N, A194V/A467N/L658V, A194V/A467N/L658V/N743T, A194V/A467N/R704K, A194V/A467N/N743T, A194V/L658V, A194V/N743T, L242G, R252L/R358N/E696T/R704K, R252L/G412A/L658V, R252L/A467N/E696T, R258L/P347H, R284A, E304G, E304V, L331G, R434K, H489R, Q543A, A560G, N592A, L658E, L666R, H685F, E717T, E721M, E721R, and 814M, or combinations thereof, wherein the amino acid positions are relative to the reference sequence of SEQ ID NO: 994.

The present invention further provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 1858, or to a reference sequence of SEQ ID NO: 1858, wherein the polypeptide sequence comprises one or more substitutions relative to the reference sequence comprising residues 12 to 841 of SEQ ID NO: 1858, or to the reference sequence of SEQ ID NO: 1858.

In some embodiments, the engineered polynucleotides encoding engineered DNA polymerases comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 1858 or to the reference sequence of SEQ ID NO: 1858.

In some embodiments, the present invention provides an engineered polynucleotide encoding an engineered DNA polymerase comprising one or more substitutions or substitution sets selected from 194/252/269/304/467, 194/252/304, 194/252/304/358/467/658, 194/252/304/467, 194/252/358/489, 194/252/467, 194/269, 194/269/304, 194/269/304/658/717, 194/269/717, 194/304/358/467/489, 194/467/721, 194/536/658, 194/721, 252, 252/269/304/743, 252/304/489/696/717, 252/304/658, 252/358/467/489/685, 252/685, 269, 269/304, 269/304/467, 269/304/658, 269/658, 269/721, 304, 304/358/467/592, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from 194V/252L/269G/304G/467N, 194V/252L/304G, 194V/252L/304G/358N/467N/658E, 194V/252L/304G/467N, 194V/252L/358N/489R, 194V/252L/467N, 194V/269G, 194V/269G/304G, 194V/269G/304V/658V/717T, 194V/269G/717T, 194V/304V/358N/467N/489R, 194V/467N/721R, 194V/536G/658E, 194V/721R, 252L, 252L/269G/304V/743T, 252L/304G/489R/696T/717T, 252L/304G/658V, 252L/358N/467N/489R/685F, 252L/685F, 269G, 269G/304G, 269G/304G/658E, 269G/304V/467N, 269G/658V, 269G/721R, 304G, 304G/358N/467N/592A, 304G/467N/489R/717T, 304G/489R/658V/721R, 304G/685F, 304V, 304V/489R/658E, 358N/658V, 489R, 489R/696T/743T, 685F, 696T, 717T, and 721R, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from A194V/R252L/R269G/E304G/A467N, A194V/R252L/E304G, A194V/R252L/E304G/R358N/A467N/L658E, A194V/R252L/E304G/A467N, A194V/R252L/R358N/H489R, A194V/R252L/A467N, A194V/R269G, A194V/R269G/E304G, A194V/R269G/E304V/L658V/E717T, A194V/R269G/E717T, A194V/E304V/R358N/A467N/H489R, A194V/A467N/E721R, A194V/P536G/L658E, A194V/E721R, R252L, R252L/R269G/E304V/N743T, R252L/E304G/H489R/E696T/E717T, R252L/E304G/L658V, R252L/R358N/A467N/H489R/H685F, R252L/H685F, R269G, R269G/E304G, R269G/E304G/L658E, R269G/E304V/A467N, R269G/L658V, R269G/E721R, E304G, E304G/R358N/A467N/N592A, E304G/A467N/H489R/E717T, E304G/H489R/L658V/E721R, E304G/H685F, E304V, E304V/H489R/L658E, R358N/L658V, H489R, H489R/E696T/N743T, H685F, E696T, E717T, and E721R, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from 252, 252/304, 252/304/489/696/717, 252/304/658, 252/358/467/489/685, 252/685, 269, 269/304/467, 269/304/489/685, 269/467/489/658/685, 269/658, 269/721, 304, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from 252L, 252L/304G/489R/696T/717T, 252L/304G/658V, 252L/304V, 252L/358N/467N/489R/685F, 252L/685F, 269G, 269G/304G/489R/685F, 269G/304V/467N, 269G/467N/489R/658V/685F, 269G/658V, 269G/721R, 304G/467N/489R/717T, 304G/489R/658V/721R, 304G/685F, 304V, 304V/489R/658E, 358N/658V, 489R, 489R/696T/743T, 685F, 696T, 717T, and 721R, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from R252L, R252L/E304G/H489R/E696T/E717T, R252L/E304G/L658V, R252L/E304V, R252L/R358N/A467N/H489R/H685F, R252L/H685F, R269G, R269G/E304G/H489R/H685F, R269G/E304V/A467N, R269G/A467N/H489R/L658V/H685F, R269G/L658V, R269G/E721R, E304G/A467N/H489R/E717T, E304G/H489R/L658V/E721R, E304G/H685F, E304V, E304V/H489R/L658E, R358N/L658V, H489R, H489R/E696T/N743T, H685F, E696T, E717T, and E721R, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from 252/685, 269, 269/658, 304/467/489/717, 304/489/658/721, 304/685, 358/658, 489, 685, 696, 717, and 721, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from 252L/685F, 269G, 269G/658V, 304G/467N/489R/717T, 304G/489R/658V/721R, 304G/685F, 358N/658V, 489R, 685F, 696T, 717T, and 721R, or combinations thereof, wherein the amino acid positions are relative to the reference sequence of SEQ ID NO: 1858.

In some embodiments, the present invention provides an engineered polynucleotide encoding an engineered DNA polymerase comprising one or more substitutions or substitution sets selected from R252L/H685F, R269G, R269G/L658V, E304G/A467N/H489R/E717T, E304G/H489R/L658V/E721R, E304G/H685F, R358N/L658V, H489R, H685F, E696T, E717T, and E721R, or combinations thereof, wherein the amino acid positions are relative to the reference sequence of SEQ ID NO: 1858.

The present invention also provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 2176, or to a reference sequence of SEQ ID NO: 2176, wherein the polypeptide sequence comprises one or more substitutions relative to the reference sequence comprising residues 12 to 841 of SEQ ID NO: 2176, or to the reference sequence of SEQ ID NO: 2176.

In some embodiments, the present invention provides an engineered polynucleotide encoding an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2176 or to the reference sequence of SEQ ID NO: 2176.

In some embodiments, the present invention provides an engineered polynucleotide encoding an engineered DNA polymerase comprising one or more substitutions or substitution sets selected from 40, 42, 45, 45/65/243/268, 45/109/340/605/814, 45/221/243/340/423, 45/243/312/331/451, 45/243/312/331/557, 45/243/312/340/423/543/814, 45/243/814, 45/268/312/605/748/749/814, 45/268/489/557/748/749, 45/312/340/605, 45/340/423/814, 45/340/557, 45/423, 45/431, 45/451, 45/475/557/748/749, 45/489/557/749, 45/621, 45/699/748/749, 57, 61, 64/293, 65, 65/233/543/621, 65/489/543, 81, 94, 102, 109, 109/340/489/510/659, 118, 124, 145, 151, 177, 186, 194, 194/269/489, 194/358, 210, 211, 221, 223, 227, 227/443/592/617, 227/443/756, 233, 242, 243, 243/268, 252, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 289, 292, 292/430, 298, 300, 301, 301/538/546/621/814, 302, 302/340/489/494/510/748/814, 302/340/494, 302/494, 302/494/525, 302/494/543/659/814, 304/489, 312, 312/331/605/814, 312/340/494, 316, 323, 329, 331, 331/605, 340, 340/489, 340/489/494/543/659/814, 340/489/699, 350, 352, 352/451/489/543/557/721, 355, 355/430, 358/467, 371, 397, 406, 407, 420, 423, 431, 441, 451, 463, 466, 475, 481, 488, 489, 489/494, 489/510, 489/621/814, 489/658, 492, 494, 501, 510, 510/748, 519, 521, 530, 533, 534, 536, 538, 543, 543/699/814, 553, 557, 557/605/699/721, 560, 597, 605, 621, 639, 644, 659, 660, 666, 670, 685, 686, 692, 694, 696, 699, 704, 717, 721, 722, 726, 732, 738, 743, 748, 748/749, 749, 756, 802, 804, 814, 827, 829, and 834, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from 40C, 40S, 42V, 45G, 45L, 45L/621R, 45M, 45S, 45S/65N/243N/268Q, 45S/109H/340T/605K/814T, 45S/221D/243N/340T/423E, 45S/243N/312Q/331A/451G, 45S/243N/312Q/331A/557R, 45S/243N/312Q/340T/423E/543A/814T, 45S/243N/814T, 45S/268G/312Q/605K/748G/749K/814T, 45S/268Q/489R/557R/748G/749K, 45S/312Q/340T/605K, 45S/340T/423E/814T, 45S/340T/557R, 45S/423E, 45S/431T, 45S/451G, 45S/475H/557R/748G/749K, 45S/489R/557R/749K, 45S/699Q/748G/749K, 45V, 571, 57R, 61G, 64E/293H, 65C, 65K/489R/543A, 65N, 65N/233S/543V/621R, 65S, 81W, 94K, 94L, 94M, 94R, 102D, 102E, 102G, 102T, 109C, 109H, 109H/340T/489R/510R/659G, 109T, 109Y, 118L, 118T, 118V, 124A, 145F, 151S, 177F, 186M, 194G, 194V/269G/489R, 194V/358N, 210G, 210R, 210S, 211L, 211R, 221C, 221D, 221T, 223D, 227E, 227E/443D/592S/617V, 227E/443D/756R, 233E, 233L, 233R, 233S, 242Q, 242V, 243N, 243N/268G, 243S, 252L, 268E, 268G, 268G/423E/814T, 268G/431T/605K/749K, 268K, 268Q, 268Q/312Q/331A/340T/699Q, 268Q/312Q/331A/423E/451G/557R/605K, 268Q/340T/352Q/423E/451G, 268Q/423E, 268Q/423E/431T/543A/557R/605K/814T, 268Q/423E/814T, 268V, 269G, 269G/658E, 2691, 269L, 269M, 269S, 269V, 272L, 272V, 280E, 280G, 280K, 283S, 284E, 284G, 289E, 292E, 292E/430H, 292G, 292M, 292R, 292W, 298G, 298I, 300E, 300G, 300L, 300M, 300S, 300T, 301C, 301E, 301G, 301G/538M/546A/621R/814T, 302L/340T/489R/494S/510R/748G/814T, 302V, 302V/340T/494V, 302V/494V, 302V/494V/525R, 302V/494V/543V/659G/814T, 304G/489R, 312D, 312Q, 312Q/331A/605K/814T, 312Q/340T/494V, 316T, 323C, 329M, 329S, 329T, 329V, 331A, 331K, 331M, 331S, 331S/605K, 331V, 340A, 340K, 340L, 340T, 340T/489R, 340T/489R/494V/543A/659G/814T, 340T/489R/699Q, 340V, 350C, 350G, 350S, 352Q, 352Q/451G/489R/543V/557R/721R, 355C, 355L/430R, 355T, 355V, 358N/467N, 371C, 371S, 397V, 406G, 406L, 406W, 407L, 407V, 420H, 423E, 423R, 431T, 431V, 441L, 441M, 451G, 463L, 463W, 466H, 466K, 466M, 475C, 475H, 481A, 481S, 488A, 488R, 488S, 489R, 489R/494V, 489R/510R, 489R/621R/814T, 489R/658E, 492A, 494H, 494R, 494V, 494Y, 501V, 510D, 510L, 510M, 510R, 510R/748G, 519F, 519L, 519V, 521W, 530D, 530E, 530G, 530T, 530V, 530W, 533P, 533T, 534A, 534G, 534R, 534W, 536D, 536G, 536T, 538M, 543A, 543L, 543V/699S/814T, 553Q, 553V, 557L, 557M, 557R, 557R/605K/699Q/721R, 557S, 560M, 560S, 560V, 597G, 597N, 597S, 597V, 605K, 621D, 621E, 621G, 621K, 621Q, 621R, 621T, 639K, 644R, 659A, 659C, 659T, 660G, 660V, 666S, 666W, 670M, 685G, 685V, 686G, 692R, 694T, 696Q, 699G, 699Q, 699S, 704R, 717D, 721A, 721R, 722V, 722W, 726M, 732M, 738P, 743G, 748G, 748G/749K, 749G, 749K, 749S, 749T, 756I, 756L, 802C, 802R, 804A, 804G, 814G, 814K, 814M, 814Q, 814R, 827R, 829R, and 834L, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from G40C, G40S, T42V, R45G, R45L, R45L/S621R, R45M, R45S, R45S/E65N/A243N/R268Q, R45S/E109H/R340T/R605K/E814T, R45S/S221D/A243N/R340T/A423E, R45S/A243N/E312Q/L331A/A451G, R45S/A243N/E312Q/L331A/P557R, R45S/A243N/E312Q/R340T/A423E/Q543A/E814T, R45S/A243N/E814T, R45S/R268G/E312Q/R605K/S748G/V749K/E814T, R45S/R268Q/H489R/P557R/S748G/V749K, R45S/E312Q/R340T/R605K, R45S/R340T/A423E/E814T, R45S/R340T/P557R, R45S/A423E, R45S/G431T, R45S/A451G, R45S/E475H/P557R/S748G/V749K, R45S/H489R/P557R/V749K, R45S/A699Q/S748G/V749K, R45V, K57I, K57R, K61G, K64E/E293H, E65C, E65K/H489R/Q543A, E65N, E65N/K233S/Q543V/S621R, E65S, F81W, A94K, A94L, A94M, A94R, R102D, R102E, R102G, R102T, E109C, E109H, E109H/R340T/H489R/P510R/S659G, E109T, E109Y, R118L, R118T, R118V, F124A, Q145F, R151S, W177F, Q186M, A194G, A194V/R269G/H489R, A194V/R358N, K210G, K210R, K210S, T211L, T211R, S221C, S221D, S221T, E223D, K227E, K227E/E443D/N592S/A617V, K227E/E443D/M756R, K233E, K233L, K233R, K233S, L242Q, L242V, A243N, A243N/R268G, A243S, R252L, R268E, R268G, R268G/A423E/E814T, R268G/G431T/R605K/V749K, R268K, R268Q, R268Q/E312Q/L331A/R340T/A699Q, R268Q/E312Q/L331A/A423E/A451G/P557R/R605K, R268Q/R340T/R352Q/A423E/A451G, R268Q/A423E, R268Q/A423E/G431T/Q543A/P557R/R605K/E814T, R268Q/A423E/E814T, R268V, R269G, R269G/L658E, R269I, R269L, R269M, R269S, R269V, E272L, E272V, A280E, A280G, A280K, E283S, R284E, R284G, S289E, H292E, H292E/Q430H, H292G, H292M, H292R, H292W, E298G, E298I, P300E, P300G, P300L, P300M, P300S, P300T, L301C, L301E, L301G, L301G/V538M/E546A/S621R/E814T, P302L/R340T/H489R/N494S/P510R/S748G/E814T, P302V, P302V/R340T/N494V, P302V/N494V, P302V/N494V/A525R, P302V/N494V/Q543V/S659G/E814T, E304G/H489R, E312D, E312Q, E312Q/L331A/R605K/E814T, E312Q/R340T/N494V, L316T, P323C, E329M, E329S, E329T, E329V, L331A, L331K, L331M, L331S, L331S/R605K, L331V, R340A, R340K, R340L, R340T, R340T/H489R, R340T/H489R/N494V/Q543A/S659G/E814T, R340T/H489R/A699Q, R340V, A350C, A350G, A350S, R352Q, R352Q/A451G/H489R/Q543V/P557R/E721R, F355C, F355L/Q430R, F355T, F355V, R358N/A467N, R371C, R371S, E397V, E406G, E406L, E406W, W407L, W407V, R420H, A423E, A423R, G431T, G431V, E441L, E441M, A451G, A463L, A463W, E466H, E466K, E466M, E475C, E475H, E481A, E481S, G488A, G488R, G488S, H489R, H489R/N494V, H489R/P510R, H489R/S621R/E814T, H489R/L658E, R492A, N494H, N494R, N494V, N494Y, R501V, P510D, P510L, P510M, P510R, P510R/S748G, G519F, G519L, G519V, R521W, A530D, A530E, A530G, A530T, A530V, A530W, E533P, E533T, V534A, V534G, V534R, V534W, P536D, P536G, P536T, V538M, Q543A, Q543L, Q543V/A699S/E814T, T553Q, T553V, P557L, P557M, P557R, P557R/R605K/A699Q/E721R, P557S, A560M, A560S, A560V, T597G, T597N, T597S, T597V, R605K, S621D, S621E, S621G, S621K, S621Q, S621R, S621T, R639K, G644R, S659A, S659C, S659T, A660G, A660V, L666S, L666W, A670M, F685G, F685V, R686G, G692R, P694T, E696Q, A699G, A699Q, A699S, K704R, E717D, E721A, E721R, E722V, E722W, R726M, L732M, Y738P, N743G, S748G, S748G/V749K, V749G, V749K, V749S, V749T, M756I, M756L, K802C, K802R, R804A, R804G, E814G, E814K, E814M, E814Q, E814R, E827R, E829R, and E834L, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from 45, 45/65/243/268, 45/109/243/423/543/605/699/748/749/814, 45/109/340/605/814, 45/221/243/340/423, 45/243/312/331/451, 45/243/312/331/557, 45/243/312/340/423/543/814, 45/243/312/605, 45/243/814, 45/268/312/605/748/749/814, 45/268/489/543/557/659/721, 45/268/489/557/748/749, 45/312, 45/312/340/605, 45/340/423/814, 45/340/475/557/814, 45/340/557, 45/423, 45/431, 45/451, 45/475/557/748/749, 45/489/557/749, 45/621, 45/699/748/749, 64/293, 65, 65/489/543, 81, 94, 102, 109, 109/340/489/510/659, 109/423, 118, 120, 151, 162, 194, 211, 221, 233, 243, 243/268, 245, 252, 258, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 292, 298, 300, 301, 301/538/546/621/814, 302/340/489/494/510/748/814, 302/340/489/494/525, 302/340/494, 302/494, 302/494/525, 304/489, 312, 312/331/605/814, 312/423/431/451/489, 312/494/525/543/659/748/814, 323, 329, 331, 331/605, 340, 340/489, 340/489/494/543/659/814, 340/489/699, 350, 352, 352/451/489/543/557/721, 355, 355/430, 358/467, 406, 407, 423, 423/463/605, 431, 451, 463, 466, 467, 475, 476, 481, 489, 489/494, 489/494/659, 489/510, 489/658, 494, 501, 510, 510/748, 519, 530, 533, 534, 538, 543, 543/699/814, 557, 557/605/699/721, 560, 597, 605, 621, 659, 666, 694, 696, 699, 721, 722, 748, 748/749, 749, 756, 802, and 814, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from 45G, 45L, 45L/621R, 45S, 45S/65N/243N/268Q, 45S/109H/243N/423E/543V/605K/699Q/748G/749K/814T, 45S/109H/340T/605K/814T, 45S/221D/243N/340T/423E, 45S/243N/312Q/331A/451G, 45S/243N/312Q/331A/557R, 45S/243N/312Q/340T/423E/543A/814T, 45S/243N/312Q/605K, 45S/243N/814T, 45S/268G/312Q/605K/748G/749K/814T, 45S/268Q/489R/543V/557R/659T/721R, 45S/268Q/489R/557R/748G/749K, 45S/312Q, 45S/312Q/340T/605K, 45S/340T/423E/814T, 45S/340T/475H/557R/814T, 45S/340T/557R, 45S/423E, 45S/431T, 45S/451G, 45S/475H/557R/748G/749K, 45S/489R/557R/749K, 45S/699Q/748G/749K, 45V, 64H/293D, 65C, 65K/489R/543A, 65N, 65S, 81W, 94K, 102P, 109H, 109H/340T/489R/510R/659G, 109H/423E, 118L, 118V, 120D, 151G, 162T, 194G, 211R, 221D, 233R, 243N, 243N/268G, 243S, 245T, 252L, 258L, 268E, 268G, 268G/423E/814T, 268G/431T/605K/749K, 268Q, 268Q/312Q/331A/340T/699Q, 268Q/312Q/331A/423E/451G/557R/605K, 268Q/340T/352Q/423E/451G, 268Q/423E, 268Q/423E/431T/543A/557R/605K/814T, 268Q/423E/814T, 269G, 269G/658E, 2691, 272V, 280G, 283A, 283L, 283S, 284G, 292R, 298D, 298I, 300E, 300G, 300L, 300M, 300S, 300T, 301C, 301E, 301G, 301G/538M/546A/621R/814T, 302L/340T/489R/494S/510R/748G/814T, 302L/340T/489R/494V/525R, 302V/340T/494V, 302V/494V, 302V/494V/525R, 304G/489R, 312Q, 312Q/331A/605K/814T, 312Q/423E/431T/451G/489R, 312Q/494V/525R/543V/659G/748G/814T, 323W, 329M, 329S, 331A, 331S/605K, 331V, 340L, 340T, 340T/489R, 340T/489R/494V/543A/659G/814T, 340T/489R/699Q, 350C, 350G, 350Y, 352Q, 352Q/451G/489R/543V/557R/721R, 355C, 355L/430R, 355T, 358N/467N, 406G, 406L, 407V, 423E, 423E/463W/605K, 431T, 431V, 451G, 463W, 466H, 466M, 467N, 475C, 475H, 475L, 476V, 481S, 489R, 489R/494V, 489R/494V/659G, 489R/510R, 489R/658E, 494Y, 501V, 510M, 510R, 510R/748G, 519F, 519L, 530D, 530E, 530T, 530W, 533T, 534A, 534G, 538M, 543L, 543V, 543V/699S/814T, 557R, 557R/605K/699Q/721R, 560S, 597G, 597N, 597S, 605K, 621E, 621K, 621R, 659T, 666E, 666S, 666W, 694T, 696Q, 699G, 699Q, 699S, 721A, 721R, 722V, 748G, 748G/749K, 749G, 749K, 749S, 756L, 802C, 802R, 814K, and 814T, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from R45G, R45L, R45L/S621R, R45S, R45S/E65N/A243N/R268Q, R45S/E109H/A243N/A423E/Q543V/R605K/A699Q/S748G/V749K/E814T, R45S/E109H/R340T/R605K/E814T, R45S/S221D/A243N/R340T/A423E, R45S/A243N/E312Q/L331A/A451G, R45S/A243N/E312Q/L331A/P557R, R45S/A243N/E312Q/R340T/A423E/Q543A/E814T, R45S/A243N/E312Q/R605K, R45S/A243N/E814T, R45S/R268G/E312Q/R605K/S748G/V749K/E814T, R45S/R268Q/H489R/Q543V/P557R/S659T/E721R, R45S/R268Q/H489R/P557R/S748G/V749K, R45S/E312Q, R45S/E312Q/R340T/R605K, R45S/R340T/A423E/E814T, R45S/R340T/E475H/P557R/E814T, R45S/R340T/P557R, R45S/A423E, R45S/G431T, R45S/A451G, R45S/E475H/P557R/S748G/V749K, R45S/H489R/P557R/V749K, R45S/A699Q/S748G/V749K, R45V, K64H/E293D, E65C, E65K/H489R/Q543A, E65N, E65S, F81W, A94K, R102P, E109H, E109H/R340T/H489R/P510R/S659G, E109H/A423E, R118L, R118V, E120D, R151G, S162T, A194G, T211R, S221D, K233R, A243N, A243N/R268G, A243S, L245T, R252L, R258L, R268E, R268G, R268G/A423E/E814T, R268G/G431T/R605K/V749K, R268Q, R268Q/E312Q/L331A/R340T/A699Q, R268Q/E312Q/L331A/A423E/A451G/P557R/R605K, R268Q/R340T/R352Q/A423E/A451G, R268Q/A423E, R268Q/A423E/G431T/Q543A/P557R/R605K/E814T, R268Q/A423E/E814T, R269G, R269G/L658E, R269I, E272V, A280G, E283A, E283L, E283S, R284G, H292R, E298D, E298I, P300E, P300G, P300L, P300M, P300S, P300T, L301C, L301E, L301G, L301G/V538M/E546A/S621R/E814T, P302L/R340T/H489R/N494S/P510R/S748G/E814T, P302L/R340T/H489R/N494V/A525R, P302V/R340T/N494V, P302V/N494V, P302V/N494V/A525R, E304G/H489R, E312Q, E312Q/L331A/R605K/E814T, E312Q/A423E/G431T/A451G/H489R, E312Q/N494V/A525R/Q543V/S659G/S748G/E814T, P323W, E329M, E329S, L331A, L331S/R605K, L331V, R340L, R340T, R340T/H489R, R340T/H489R/N494V/Q543A/S659G/E814T, R340T/H489R/A699Q, A350C, A350G, A350Y, R352Q, R352Q/A451G/H489R/Q543V/P557R/E721R, F355C, F355L/Q430R, F355T, R358N/A467N, E406G, E406L, W407V, A423E, A423E/A463W/R605K, G431T, G431V, A451G, A463W, E466H, E466M, A467N, E475C, E475H, E475L, M476V, E481S, H489R, H489R/N494V, H489R/N494V/S659G, H489R/P510R, H489R/L658E, N494Y, R501V, P510M, P510R, P510R/S748G, G519F, G519L, A530D, A530E, A530T, A530W, E533T, V534A, V534G, V538M, Q543L, Q543V, Q543V/A699S/E814T, P557R, P557R/R605K/A699Q/E721R, A560S, T597G, T597N, T597S, R605K, S621E, S621K, S621R, S659T, L666E, L666S, L666W, P694T, E696Q, A699G, A699Q, A699S, E721A, E721R, E722V, S748G, S748G/V749K, V749G, V749K, V749S, M756L, K802C, K802R, E814K, and E814T, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from 45, 45/65/243/268, 45/221/243/340/423, 45/268/312/605/748/749/814, 45/268/489/557/748/749, 45/451, 45/475/557/748/749, 45/489/557/749, 45/621, 45/699/748/749, 65, 65/489/543, 81, 94, 102, 109, 109/340/489/510/659, 221, 227/443/756, 252, 268, 268/312/331/340/699, 268/312/331/423/451/557/605, 268/423/431/543/557/605/814, 269, 283, 300, 301, 301/538/546/621/814, 302/340/489/494/510/748/814, 302/340/489/494/525, 302/494/525, 304/489, 312, 312/494/525/543/659/748/814, 329, 331, 340, 340/489/699, 352, 358/467, 406, 431, 451, 463, 466, 475, 481, 489, 489/510, 489/658, 510, 510/748, 530, 543, 543/699/814, 557, 557/605/699/721, 560, 605, 659, 666, 696, 699, 721, 722, 748, 748/749, 749, 802, and 814, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from 45L/621R, 45S, 45S/65N/243N/268Q, 45S/221D/243N/340T/423E, 45S/268G/312Q/605K/748G/749K/814T, 45S/268Q/489R/557R/748G/749K, 45S/451G, 45S/475H/557R/748G/749K, 45S/489R/557R/749K, 45S/699Q/748G/749K, 65K/489R/543A, 65N, 81W, 94K, 102P, 109H, 109H/340T/489R/510R/659G, 221D, 227E/443D/756R, 252L, 268G, 268Q, 268Q/312Q/331A/340T/699Q, 268Q/312Q/331A/423E/451G/557R/605K, 268Q/423E/431T/543A/557R/605K/814T, 269G, 283L, 300M, 300S, 300T, 301G, 301G/538M/546A/621R/814T, 302L/340T/489R/494S/510R/748G/814T, 302L/340T/489R/494V/525R, 302V/494V/525R, 304G/489R, 312Q, 312Q/494V/525R/543V/659G/748G/814T, 329S, 331A, 340T, 340T/489R/699Q, 352Q, 358N/467N, 406L, 431T, 451G, 463W, 466M, 475C, 475H, 481S, 489R, 489R/510R, 489R/658E, 510R, 510R/748G, 530D, 530E, 530T, 543V, 543V/699S/814T, 557R, 557R/605K/699Q/721R, 560S, 605K, 659T, 666E, 666S, 666W, 696Q, 699Q, 721R, 722V, 748G, 748G/749K, 749K, 802C, and 814T, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from R45L/S621R, R45S, R45S/E65N/A243N/R268Q, R45S/S221D/A243N/R340T/A423E, R45S/R268G/E312Q/R605K/S748G/V749K/E814T, R45S/R268Q/H489R/P557R/S748G/V749K, R45S/A451G, R45S/E475H/P557R/S748G/V749K, R45S/H489R/P557R/V749K, R45S/A699Q/S748G/V749K, E65K/H489R/Q543A, E65N, F81W, A94K, R102P, E109H, E109H/R340T/H489R/P510R/S659G, S221D, K227E/E443D/M756R, R252L, R268G, R268Q, R268Q/E312Q/L331A/R340T/A699Q, R268Q/E312Q/L331A/A423E/A451G/P557R/R605K, R268Q/A423E/G431T/Q543A/P557R/R605K/E814T, R269G, E283L, P300M, P300S, P300T, L301G, L301G/V538M/E546A/S621R/E814T, P302L/R340T/H489R/N494S/P510R/S748G/E814T, P302L/R340T/H489R/N494V/A525R, P302V/N494V/A525R, E304G/H489R, E312Q, E312Q/N494V/A525R/Q543V/S659G/S748G/E814T, E329S, L331A, R340T, R340T/H489R/A699Q, R352Q, R358N/A467N, E406L, G431T, A451G, A463W, E466M, E475C, E475H, E481S, H489R, H489R/P510R, H489R/L658E, P510R, P510R/S748G, A530D, A530E, A530T, Q543V, Q543V/A699S/E814T, P557R, P557R/R605K/A699Q/E721R, A560S, R605K, S659T, L666E, L666S, L666W, E696Q, A699Q, E721R, E722V, S748G, S748G/V749K, V749K, K802C, and E814T, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from 45/65/243/268, 45/268/312/605/748/749/814, 45/268/489/557/748/749, 45/451, 45/475/557/748/749, 45/489/557/749, 45/621, 45/699/748/749, 57, 65, 65/489/543, 81, 94, 109, 221, 243, 268, 268/312/331/340/699, 268/312/331/423/451/557/605, 268/423/431/543/557/605/814, 269, 300, 301, 302/340/489/494/510/748/814, 302/494/525, 304/489, 312, 331, 331/605, 340, 340/489/699, 352, 358/467, 406, 423, 431, 451, 463, 466, 475, 481, 489, 489/510, 489/658, 494, 510, 510/748, 530, 543, 543/699/814, 557, 557/605/699/721, 560, 597, 605, 659, 666, 696, 699, 721, 748, 748/749, 749, 802, and 814, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from 45L/621R, 45S/65N/243N/268Q, 45S/268G/312Q/605K/748G/749K/814T, 45S/268Q/489R/557R/748G/749K, 45S/451G, 45S/475H/557R/748G/749K, 45S/489R/557R/749K, 45S/699Q/748G/749K, 57R, 65K/489R/543A, 65N, 81W, 94K, 109H, 221D, 243N, 268G, 268Q, 268Q/312Q/331A/340T/699Q, 268Q/312Q/331A/423E/451G/557R/605K, 268Q/423E/431T/543A/557R/605K/814T, 269G, 300S, 300T, 301G, 302L/340T/489R/494S/510R/748G/814T, 302V/494V/525R, 304G/489R, 312Q, 331A, 331S/605K, 340L, 340T, 340T/489R/699Q, 352Q, 358N/467N, 406L, 423E, 431T, 451G, 463W, 466M, 475C, 475H, 481S, 489R, 489R/510R, 489R/658E, 494S, 510R, 510R/748G, 530D, 530E, 530T, 543V, 543V/699S/814T, 557R, 557R/605K/699Q/721R, 560S, 597G, 597N, 605K, 659T, 666E, 666S, 666W, 696Q, 699Q, 721A, 721R, 748G, 748G/749K, 749K, 802C, 802R, and 814, or combinations thereof, wherein the amino acid positions are relative to the reference sequence of SEQ ID NO: 2176.

In some embodiments, the present invention provides an engineered polynucleotide encoding an engineered DNA polymerase comprising one or more substitutions or substitution sets selected from R45L/S621R, R45S/E65N/A243N/R268Q, R45S/R268G/E312Q/R605K/S748G/V749K/E814T, R45S/R268Q/H489R/P557R/S748G/V749K, R45S/A451G, R45S/E475H/P557R/S748G/V749K, R45S/H489R/P557R/V749K, R45S/A699Q/S748G/V749K, K57R, E65K/H489R/Q543A, E65N, F81W, A94K, E109H, S221D, A243N, R268G, R268Q, R268Q/E312Q/L331A/R340T/A699Q, R268Q/E312Q/L331A/A423E/A451G/P557R/R605K, R268Q/A423E/G431T/Q543A/P557R/R605K/E814T, R269G, P300S, P300T, L301G, P302L/R340T/H489R/N494S/P510R/S748G/E814T, P302V/N494V/A525R, E304G/H489R, E312Q, L331A, L331S/R605K, R340L, R340T, R340T/H489R/A699Q, R352Q, R358N/A467N, E406L, A423E, G431T, A451G, A463W, E466M, E475C, E475H, E481S, H489R, H489R/P510R, H489R/L658E, N494S, P510R, P510R/S748G, A530D, A530E, A530T, Q543V, Q543V/A699S/E814T, P557R, P557R/R605K/A699Q/E721R, A560S, T597G, T597N, R605K, S659T, L666E, L666S, L666W, E696Q, A699Q, E721A, E721R, S748G, S748G/V749K, V749K, K802C, K802R, and 814T, or combinations thereof, wherein the amino acid positions are relative to the reference sequence of SEQ ID NO: 2176.

The present invention also provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 2310, or to a reference sequence of SEQ ID NO: 2310, wherein the polypeptide sequence comprises one or more substitutions relative to the reference sequence comprising residues 12 to 841 of SEQ ID NO: 2310, or to the reference sequence of SEQ ID NO: 2310.

In some embodiments, the present invention provides an engineered polynucleotide encoding an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2310 or to the reference sequence of SEQ ID NO: 2310.

In some embodiments, the present invention provides an engineered polynucleotide encoding an engineered DNA polymerase comprising one or more substitutions or substitution sets selected from 18/268/302/312/605/659/699, 45/227/268/423/489/510/605, 45/227/302/423/510/605/621/699, 45/227/312/423/525/605/699, 45/268/302/423/494/621/699, 45/268/443/525/696, 45/331/489, 45/331/621, 45/340/423, 45/494/525/621/696/699, 45/605, 45/696/699, 65/685, 227, 227/268/340/451, 227/268/423/621, 227/302, 227/312/489/494/621, 227/331, 227/331/423/605, 227/331/605/659, 227/340/423/605, 227/340/621, 227/423, 227/423/451, 227/451/525/621/659/699/721, 227/525, 227/543/659/699, 227/605/721, 227/699, 268/302/340/358/605/699, 268/302/423/605, 268/340, 268/340/423/443/525/621/659, 268/340/423/621, 268/340/525/605, 268/423, 268/423/478/489/525/605/699, 268/423/489/494/605/659, 268/423/510/605, 268/423/605/699, 268/423/699, 268/467, 268/467/489, 268/467/489/510/699, 268/467/489/699, 268/467/489/699/814, 268/467/510/699/814, 268/489/494/605/699, 268/489/699, 268/510, 268/510/699/814, 268/525, 268/525/605, 268/543, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/451, 331/525/605/699, 331/605/699, 340/451/494/525, 358/467/489/699, 358/467/699, 358/467/699/814, 423/443/451/494/510, 423/451/605/659/699, 423/489/494/529/605/621/696/699, 423/494, 423/525, 423/605/621, 451/510, 467, 467/489, 467/489/510/543/699/814, 467/489/699, 467/489/699/814, 467/489/814, 467/510/699, 467/510/699/814, 467/510/814, 467/699, 484, 489, 489/494/605/721, 489/510/525/605/699, 489/699, 489/699/814, 494/605/699, 494/659, 494/699, 498, 503, 510, 510/605/659, 510/699, 510/699/814, 524, 526, 527, 531, 540, 542, 543/699, 543/699/814, 547, 569, 577/614, 605, 605/621/659/696/699, 605/621/696/699, 621, 659/699, 673, 684, 685, 685/714, 699, 699/814, 702, and 716, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/227E/312Q/423E/525R/605K/699Q, 45L/268Q/302L/423E/494S/621R/699S, 45L/268Q/443D/525R/696Q, 45L/331A/489R, 45L/331A/621R, 45L/340T/423E, 45L/494V/525R/621R/696Q/699Q, 45L/605K, 45L/696Q/699Q, 65K/685R, 227E, 227E/268G/423E/621R, 227E/268Q/340T/451G, 227E/302L, 227E/312Q/489R/494S/621R, 227E/331A/423E/605K, 227E/331S, 227E/331S/605K/659G, 227E/340T/423E/605K, 227E/340T/621R, 227E/423E, 227E/423E/451G, 227E/451G/525R/621R/659G/699Q/721R, 227E/525R, 227E/543V/659G/699S, 227E/605K/721R, 227E/699S, 268G/302L/423E/605K, 268G/340T, 268G/423E/478H/489R/525R/605K/699Q, 268G/423E/510R/605K, 268G/423E/699Q, 268G/621R, 268Q/302L/340T/358N/605K/699Q, 268Q/340T/423E/443D/525R/621R/659G, 268Q/340T/423E/621R, 268Q/340T/525R/605K, 268Q/423E, 268Q/423E/489R/494S/605K/659G, 268Q/423E/605K/699S, 268Q/467N, 268Q/467N/489R, 268Q/467N/489R/510R/699Q, 268Q/467N/489R/699Q, 268Q/467N/489R/699S/814T, 268Q/467N/510R/699S/814T, 268Q/489R/494S/605K/699Q, 268Q/489R/699Q, 268Q/510R, 268Q/510R/699Q/814T, 268Q/525R, 268Q/525R/605K, 268Q/543V, 268Q/543V/699Q, 268Q/605K/621R/721R, 268Q/699Q, 268Q/699S, 302L/423E, 302L/423E/605K/621R, 302L/543V/621R/696Q/699Q, 302L/605K/621R/721R, 312Q/331A/451G/621R/696Q/699Q, 312Q/423E/621R, 312Q/510R/605K, 331A/423E/527F, 331A/525R/605K/699Q, 331A/605K/699Q, 331S/423E, 331S/451G, 340T/451G/494V/525R, 358N/467N/489R/699Q, 358N/467N/699Q, 358N/467N/699Q/814T, 423E/443D/451G/494S/510R, 423E/451G/605K/659G/699Q, 423E/489R/494S/529D/605K/621R/696Q/699Q, 423E/494S, 423E/525R, 423E/605K/621R, 451G/510R, 467N, 467N/489R, 467N/489R/510R/543V/699S/814T, 467N/489R/699Q, 467N/489R/699S/814T, 467N/489R/814T, 467N/510R/699Q, 467N/510R/699Q/814T, 467N/510R/699S/814T, 467N/510R/814T, 467N/699Q, 467N/699S, 484V, 489R, 489R/494S/605K/721R, 489R/510R/525R/605K/699Q, 489R/699Q, 489R/699Q/814T, 489R/699S, 494S/659G, 494S/699S, 494V/605K/699Q, 498M, 498T, 503I, 510R, 510R/605K/659G, 510R/699Q, 510R/699Q/814T, 510R/699S/814T, 524D, 524Q, 526Q, 527R, 531V, 540S, 542R, 543V/699Q, 543V/699Q/814T, 543V/699S/814T, 547M, 569V, 577S/614C, 605K, 605K/621R/659G/696Q/699S, 605K/621R/696Q/699S, 621R, 659G/699Q, 673K, 684P, 685K, 685K/714T, 685R, 699Q, 699Q/814T, 702K, and 716R, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/K227E/E312Q/A423E/A525R/R605K/A699Q, S45L/R268Q/P302L/A423E/N494S/S621R/A699S, S45L/R268Q/E443D/A525R/E696Q, S45L/L331A/H489R, S45L/L331A/S621R, S45L/R340T/A423E, S45L/N494V/A525R/S621R/E696Q/A699Q, S45L/R605K, S45L/E696Q/A699Q, E65K/F685R, K227E, K227E/R268G/A423E/S621R, K227E/R268Q/R340T/A451G, K227E/P302L, K227E/E312Q/H489R/N494S/S621R, K227E/L331A/A423E/R605K, K227E/L331S, K227E/L331S/R605K/S659G, K227E/R340T/A423E/R605K, K227E/R340T/S621R, K227E/A423E, K227E/A423E/A451G, K227E/A451G/A525R/S621R/S659G/A699Q/E721R, K227E/A525R, K227E/Q543V/S659G/A699S, K227E/R605K/E721R, K227E/A699S, R268G/P302L/A423E/R605K, R268G/R340T, R268G/A423E/R478H/H489R/A525R/R605K/A699Q, R268G/A423E/P510R/R605K, R268G/A423E/A699Q, R268G/S621R, R268Q/P302L/R340T/R358N/R605K/A699Q, R268Q/R340T/A423E/E443D/A525R/S621R/S659G, R268Q/R340T/A423E/S621R, R268Q/R340T/A525R/R605K, R268Q/A423E, R268Q/A423E/H489R/N494S/R605K/S659G, R268Q/A423E/R605K/A699S, R268Q/A467N, R268Q/A467N/H489R, R268Q/A467N/H489R/P510R/A699Q, R268Q/A467N/H489R/A699Q, R268Q/A467N/H489R/A699S/E814T, R268Q/A467N/P510R/A699S/E814T, R268Q/H489R/N494S/R605K/A699Q, R268Q/H489R/A699Q, R268Q/P510R, R268Q/P510R/A699Q/E814T, R268Q/A525R, R268Q/A525R/R605K, R268Q/Q543V, R268Q/Q543V/A699Q, R268Q/R605K/S621R/E721R, R268Q/A699Q, R268Q/A699S, P302L/A423E, P302L/A423E/R605K/S621R, P302L/Q543V/S621R/E696Q/A699Q, P302L/R605K/S621R/E721R, E312Q/L331A/A451G/S621R/E696Q/A699Q, E312Q/A423E/S621R, E312Q/P510R/R605K, L331A/A423E/V527F, L331A/A525R/R605K/A699Q, L331A/R605K/A699Q, L331S/A423E, L331S/A451G, R340T/A451G/N494V/A525R, R358N/A467N/H489R/A699Q, R358N/A467N/A699Q, R358N/A467N/A699Q/E814T, A423E/E443D/A451G/N494S/P510R, A423E/A451G/R605K/S659G/A699Q, A423E/H489R/N494S/E529D/R605K/S621R/E696Q/A699Q, A423E/N494S, A423E/A525R, A423E/R605K/S621R, A451G/P510R, A467N, A467N/H489R, A467N/H489R/P510R/Q543V/A699S/E814T, A467N/H489R/A699Q, A467N/H489R/A699S/E814T, A467N/H489R/E814T, A467N/P510R/A699Q, A467N/P510R/A699Q/E814T, A467N/P510R/A699S/E814T, A467N/P510R/E814T, A467N/A699Q, A467N/A699S, F484V, H489R, H489R/N494S/R605K/E721R, H489R/P510R/A525R/R605K/A699Q, H489R/A699Q, H489R/A699Q/E814T, H489R/A699S, N494S/S659G, N494S/A699S, N494V/R605K/A699Q, Q498M, Q498T, L503I, P510R, P510R/R605K/S659G, P510R/A699Q, P510R/A699Q/E814T, P510R/A699S/E814T, S524D, S524Q, A526Q, V527R, L531V, K540S, L542R, Q543V/A699Q, Q543V/A699Q/E814T, Q543V/A699S/E814T, L547M, L569V, A577S/R614C, R605K, R605K/S621R/S659G/E696Q/A699S, R605K/S621R/E696Q/A699S, S621R, S659G/A699Q, T673K, A684P, F685K, F685K/A714T, F685R, A699Q, A699Q/E814T, 1702K, and I716R, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from 18/268/302/312/605/659/699, 45/227/268/423/489/510/605, 45/227/302/423/510/605/621/699, 45/227/312/423/525/605/699, 45/268/302/423/494/621/699, 45/494/525/621/696/699, 227, 227/268/340/451, 227/268/423/621, 227/302, 227/312/489/494/621, 227/331, 227/331/423/605, 227/331/605/659, 227/340/423/605, 227/340/621, 227/423, 227/543/659/699, 227/605/721, 268, 268/302, 268/302/423/605, 268/340/423/443/525/621/659, 268/423/510/605, 268/423/605/699, 268/467, 268/467/489, 268/467/489/510/699, 268/467/489/699, 268/489/699, 268/510, 268/510/699/814, 268/525/605, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/605, 331/605/699, 340/451/494/525, 358/467/489/699, 358/467/699/814, 423, 423/443/451/494/510, 423/489/494/529/605/621/696/699, 423/605/621, 451/510, 467/489, 467/489/510/543/699/814, 467/489/699, 467/489/814, 467/510/699, 467/510/699/814, 467/699, 467/699/814, 484, 489, 489/510/525/605/699, 489/699, 489/699/814, 494/605/699, 498, 503, 510, 510/605/659, 510/699, 510/699/814, 524, 526, 543/699, 543/699/814, 605/621/659/696/699, 605/621/696/699, 621, and 699/814, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/227E/312Q/423E/525R/605K/699Q, 45L/268Q/302L/423E/494S/621R/699S, 45L/494V/525R/621R/696Q/699Q, 227E, 227E/268G/423E/621R, 227E/268Q/340T/451G, 227E/302L, 227E/312Q/489R/494S/621R, 227E/331A/423E/605K, 227E/331S, 227E/331S/605K/659G, 227E/340T/423E/605K, 227E/340T/621R, 227E/423E, 227E/543V/659G/699S, 227E/605K/721R, 268G/302L/423E/605K, 268G/423E/510R/605K, 268G/621R, 268Q, 268Q/302L, 268Q/340T/423E/443D/525R/621R/659G, 268Q/423E/605K/699S, 268Q/467N, 268Q/467N/489R, 268Q/467N/489R/510R/699Q, 268Q/467N/489R/699Q, 268Q/489R/699Q, 268Q/510R, 268Q/510R/699Q/814T, 268Q/525R/605K, 268Q/543V/699Q, 268Q/605K/621R/721R, 268Q/699Q, 302L/423E, 302L/423E/605K/621R, 302L/543V/621R/696Q/699Q, 302L/605K/621R/721R, 312Q/331A/451G/621R/696Q/699Q, 312Q/423E/621R, 312Q/510R/605K, 331A/423E/527F, 331A/605K, 331A/605K/699Q, 331S/423E, 331S/605K, 340T/451G/494V/525R, 358N/467N/489R/699Q, 358N/467N/699Q/814T, 423E, 423E/443D/451G/494S/510R, 423E/489R/494S/529D/605K/621R/696Q/699Q, 423E/605K/621R, 451G/510R, 467N/489R, 467N/489R/510R/543V/699S/814T, 467N/489R/699Q, 467N/489R/814T, 467N/510R/699Q, 467N/510R/699Q/814T, 467N/510R/699S/814T, 467N/699Q, 467N/699Q/814T, 467N/699S, 484V, 489R, 489R/510R/525R/605K/699Q, 489R/699Q, 489R/699Q/814T, 494V/605K/699Q, 498M, 498T, 5031, 510R, 510R/605K/659G, 510R/699Q, 510R/699Q/814T, 510R/699S/814T, 524D, 524Q, 526Q, 543V/699Q, 543V/699Q/814T, 605K/621R/659G/696Q/699S, 605K/621R/696Q/699S, 621R, and 699Q/814T, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/K227E/E312Q/A423E/A525R/R605K/A699Q, S45L/R268Q/P302L/A423E/N494S/S621R/A699S, S45L/N494V/A525R/S621R/E696Q/A699Q, K227E, K227E/R268G/A423E/S621R, K227E/R268Q/R340T/A451G, K227E/P302L, K227E/E312Q/H489R/N494S/S621R, K227E/L331A/A423E/R605K, K227E/L331S, K227E/L331S/R605K/S659G, K227E/R340T/A423E/R605K, K227E/R340T/S621R, K227E/A423E, K227E/Q543V/S659G/A699S, K227E/R605K/E721R, R268G/P302L/A423E/R605K, R268G/A423E/P510R/R605K, R268G/S621R, R268Q, R268Q/P302L, R268Q/R340T/A423E/E443D/A525R/S621R/S659G, R268Q/A423E/R605K/A699S, R268Q/A467N, R268Q/A467N/H489R, R268Q/A467N/H489R/P510R/A699Q, R268Q/A467N/H489R/A699Q, R268Q/H489R/A699Q, R268Q/P510R, R268Q/P510R/A699Q/E814T, R268Q/A525R/R605K, R268Q/Q543V/A699Q, R268Q/R605K/S621R/E721R, R268Q/A699Q, P302L/A423E, P302L/A423E/R605K/S621R, P302L/Q543V/S621R/E696Q/A699Q, P302L/R605K/S621R/E721R, E312Q/L331A/A451G/S621R/E696Q/A699Q, E312Q/A423E/S621R, E312Q/P510R/R605K, L331A/A423E/V527F, L331A/R605K, L331A/R605K/A699Q, L331S/A423E, L331S/R605K, R340T/A451G/N494V/A525R, R358N/A467N/H489R/A699Q, R358N/A467N/A699Q/E814T, A423E, A423E/E443D/A451G/N494S/P510R, A423E/H489R/N494S/E529D/R605K/S621R/E696Q/A699Q, A423E/R605K/S621R, A451G/P510R, A467N/H489R, A467N/H489R/P510R/Q543V/A699S/E814T, A467N/H489R/A699Q, A467N/H489R/E814T, A467N/P510R/A699Q, A467N/P510R/A699Q/E814T, A467N/P510R/A699S/E814T, A467N/A699Q, A467N/A699Q/E814T, A467N/A699S, F484V, H489R, H489R/P510R/A525R/R605K/A699Q, H489R/A699Q, H489R/A699Q/E814T, N494V/R605K/A699Q, Q498M, Q498T, L503I, P510R, P510R/R605K/S659G, P510R/A699Q, P510R/A699Q/E814T, P510R/A699S/E814T, S524D, S524Q, A526Q, Q543V/A699Q, Q543V/A699Q/E814T, R605K/S621R/S659G/E696Q/A699S, R605K/S621R/E696Q/A699S, S621R, and A699Q/E814T, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from 18/268/302/312/605/659/699, 45/227/268/423/489/510/605, 45/227/302/423/510/605/621/699, 45/494/525/621/696/699, 227/331/605/659, 227/605/721, 268, 268/302/423/605, 268/423/605/699, 268/467, 268/467/489, 268/467/489/510/699, 268/467/489/699, 268/489/699, 268/510, 268/605/621/721, 302/543/621/696/699, 312/331/451/621/696/699, 312/510/605, 331/423/527, 340/451/494/525, 423/489/494/529/605/621/696/699, 423/605/621, 467/489/510/543/699/814, 467/489/699, 467/489/814, 467/510/699/814, 489/510/525/605/699, 489/699, 489/699/814, 494/605/699, 510, 510/699/814, 543/699, and 543/699/814, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 45L/494V/525R/621R/696Q/699Q, 227E/331S/605K/659G, 227E/605K/721R, 268G/302L/423E/605K, 268Q, 268Q/423E/605K/699S, 268Q/467N, 268Q/467N/489R, 268Q/467N/489R/510R/699Q, 268Q/467N/489R/699Q, 268Q/489R/699Q, 268Q/510R, 268Q/605K/621R/721R, 302L/543V/621R/696Q/699Q, 312Q/331A/451G/621R/696Q/699Q, 312Q/510R/605K, 331A/423E/527F, 340T/451G/494V/525R, 423E/489R/494S/529D/605K/621R/696Q/699Q, 423E/605K/621R, 467N/489R/510R/543V/699S/814T, 467N/489R/699Q, 467N/489R/814T, 467N/510R/699Q/814T, 467N/510R/699S/814T, 489R/510R/525R/605K/699Q, 489R/699Q, 489R/699Q/814T, 494V/605K/699Q, 510R, 510R/699Q/814T, 510R/699S/814T, 543V/699Q, and 543V/699Q/814T, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, S45L/N494V/A525R/S621R/E696Q/A699Q, K227E/L331S/R605K/S659G, K227E/R605K/E721R, R268G/P302L/A423E/R605K, R268Q, R268Q/A423E/R605K/A699S, R268Q/A467N, R268Q/A467N/H489R, R268Q/A467N/H489R/P510R/A699Q, R268Q/A467N/H489R/A699Q, R268Q/H489R/A699Q, R268Q/P510R, R268Q/R605K/S621R/E721R, P302L/Q543V/S621R/E696Q/A699Q, E312Q/L331A/A451G/S621R/E696Q/A699Q, E312Q/P510R/R605K, L331A/A423E/V527F, R340T/A451G/N494V/A525R, A423E/H489R/N494S/E529D/R605K/S621R/E696Q/A699Q, A423E/R605K/S621R, A467N/H489R/P510R/Q543V/A699S/E814T, A467N/H489R/A699Q, A467N/H489R/E814T, A467N/P510R/A699Q/E814T, A467N/P510R/A699S/E814T, H489R/P510R/A525R/R605K/A699Q, H489R/A699Q, H489R/A699Q/E814T, N494V/R605K/A699Q, P510R, P510R/A699Q/E814T, P510R/A699S/E814T, Q543V/A699Q, and Q543V/A699Q/E814T, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from 18/268/302/312/605/659/699, 45/227/268/423/489/510/605, 45/227/302/423/510/605/621/699, 65/685, 227/331/605/659, 227/605/721, 268, 268/302/423/605, 268/423/605/699, 268/467, 268/467/489, 268/467/489/699, 268/489/699, 268/510, 268/605/621/721, 302/543/621/696/699, 312/331/451/621/696/699, 312/510/605, 423/605/621, 467/489/510/543/699/814, 467/489/699, 467/489/814, 467/510/699/814, 489/699, 489/699/814, 510, 510/699/814, 542, 543/699, 543/699/814, 685, and 685/714, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from 18H/268Q/302V/312Q/605K/659G/699Q, 45L/227E/268Q/423E/489R/510R/605K, 45L/227E/302L/423E/510R/605K/621R/699Q, 65K/685R, 227E/331S/605K/659G, 227E/605K/721R, 268G/302L/423E/605K, 268Q, 268Q/423E/605K/699S, 268Q/467N, 268Q/467N/489R, 268Q/467N/489R/699Q, 268Q/489R/699Q, 268Q/510R, 268Q/605K/621R/721R, 302L/543V/621R/696Q/699Q, 312Q/331A/451G/621R/696Q/699Q, 312Q/510R/605K, 423E/605K/621R, 467N/489R/510R/543V/699S/814T, 467N/489R/699Q, 467N/489R/814T, 467N/510R/699Q/814T, 467N/510R/699S/814T, 489R/699Q, 489R/699Q/814T, 510R, 510R/699S/814T, 542R, 543V/699Q, 543V/699Q/814T, 685K, 685K/714T, and 685, or combinations thereof, wherein the amino acid positions are relative to the reference sequence of SEQ ID NO: 2310.

In some embodiments, the present invention provides an engineered polynucleotide encoding an engineered DNA polymerase comprising one or more substitutions or substitution sets selected from P18H/R268Q/P302V/E312Q/R605K/S659G/A699Q, S45L/K227E/R268Q/A423E/H489R/P510R/R605K, S45L/K227E/P302L/A423E/P510R/R605K/S621R/A699Q, E65K/F685R, K227E/L331S/R605K/S659G, K227E/R605K/E721R, R268G/P302L/A423E/R605K, R268Q, R268Q/A423E/R605K/A699S, R268Q/A467N, R268Q/A467N/H489R, R268Q/A467N/H489R/A699Q, R268Q/H489R/A699Q, R268Q/P510R, R268Q/R605K/S621R/E721R, P302L/Q543V/S621R/E696Q/A699Q, E312Q/L331A/A451G/S621R/E696Q/A699Q, E312Q/P510R/R605K, A423E/R605K/S621R, A467N/H489R/P510R/Q543V/A699S/E814T, A467N/H489R/A699Q, A467N/H489R/E814T, A467N/P510R/A699Q/E814T, A467N/P510R/A699S/E814T, H489R/A699Q, H489R/A699Q/E814T, P510R, P510R/A699S/E814T, L542R, Q543V/A699Q, Q543V/A699Q/E814T, F685K, F685K/A714T, and F685R, or combinations thereof, wherein the amino acid positions are relative to the reference sequence of SEQ ID NO: 2310.

The present invention also provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 2994, or to a reference sequence of SEQ ID NO: 2994, wherein the polypeptide sequence comprises one or more substitutions relative to the reference sequence comprising residues 12 to 841 of SEQ ID NO: 2994, or to the reference sequence of SEQ ID NO: 2994.

In some embodiments, the present invention provides an engineered polynucleotide encoding an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2994 or to the reference sequence of SEQ ID NO: 2994.

In some embodiments, the present invention provides an engineered polynucleotide encoding an engineered DNA polymerase comprising one or more substitutions or substitution sets selected from 4, 6, 10, 11, 12, 14, 14/50, 14/50/56/598/725, 14/50/598/695, 14/95, 14/95/501/642, 14/139/249/433, 14/139/725, 14/167, 14/167/197/433, 14/167/197/501/725, 14/167/197/642/695, 14/167/249/433/598/642, 14/167/249/501/598/642, 14/167/249/598/695/725, 14/167/249/642, 14/167/642, 14/167/695, 14/167/725, 14/197/249/642, 14/197/642, 14/249/433/725, 14/249/598, 14/249/642, 14/249/725, 14/433/598, 14/501, 14/598, 14/598/695, 14/598/695/725, 14/642/725, 18, 18/302, 18/302/531/673/684, 18/302/547, 18/484/673, 18/484/673/684, 18/524/547, 18/547, 18/684, 29, 45/268/299/302/321/621/659, 45/268/302/621/721, 45/376/385/408/494/525/621/696, 45/423, 48, 50, 50/433/598, 50/501, 50/598, 50/598/695, 50/642, 53, 76, 77, 91, 95/167/197/598/642/695, 95/167/197/598/695/725, 95/249/433/642, 95/249/598, 95/598, 99, 105, 122, 139/167, 139/167/197/433/501/642, 139/433/598, 141, 167, 167/197, 167/197/642/695/725, 167/642, 167/725, 177, 197/642/725, 197/695, 202, 204, 209, 217, 227, 239, 247, 249/695, 249/725, 266, 268, 268/302/451/494/621, 270, 291, 291/684, 299, 299/474, 302, 302/423/451/494/621, 302/423/621, 302/451/494, 313, 346, 349, 356, 361, 370, 374, 377, 394, 404, 423, 423/451/621/696, 423/494/525, 426, 426/798, 427, 433/501/725, 436, 439/509/789, 440, 444, 445, 445/446, 446/811, 448, 448/793, 459, 461, 482, 484, 484/684, 485, 489, 494, 498/684, 509, 514, 517, 525/529/621, 533, 543/621/721, 547, 565, 574, 596, 598, 598/642, 598/642/695, 598/695, 598/725, 601, 610, 621, 621/659, 636, 642, 642/692, 659, 662, 673, 683, 684, 688, 689, 690, 695, 725, 741, 744, 744/793, 751, 801, 810, 818, 830, and 832, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from 4A, 4F, 4G, 4R, 4T, 4V, 6F, 6G, 6N, 6R, 6V, 101, 10Q, 10R, 11E, 11L, 11R, 12D, 12W, 14E, 14E/50H, 14E/50H/56V/598K/725H, 14E/50H/598K/695K, 14E/95D, 14E/95D/501K/642E, 14E/139D/249E/433K, 14E/139D/725H, 14E/167D, 14E/167D/197K/433K, 14E/167D/197K/501K/725H, 14E/167D/197K/642E/695K, 14E/167D/249E/433K/598K/642E, 14E/167D/249E/501K/598K/642E, 14E/167D/249E/598K/695K/725H, 14E/167D/249E/642E, 14E/167D/642E, 14E/167D/695K, 14E/167D/725H, 14E/197K/249E/642E, 14E/197K/642E, 14E/249E/433K/725H, 14E/249E/598K, 14E/249E/642E, 14E/249E/725H, 14E/433K/598K, 14E/501K, 14E/598K, 14E/598K/695K, 14E/598K/695K/725H, 14E/642E/725H, 18H, 18H/302V, 18H/302V/531V/673K/684P, 18H/302V/547M, 18H/484V/673K, 18H/484V/673K/684P, 18H/524D/547M, 18H/547M, 18H/684P, 29M, 29N, 29V, 29W, 45L/268G/299N/302L/321T/621R/659G, 45L/268G/302L/621R/721R, 45L/376F/385F/408A/494V/525R/621R/696Q, 45L/423E, 48M, 48V, 50H, 50H/433K/598K, 50H/501K, 50H/598K, 50H/598K/695K, 50H/642E, 53A, 53Q, 53W, 76L, 76S, 76W, 76Y, 77H, 77M, 77R, 77S, 91C, 91R, 91S, 91W, 95D/167D/197K/598K/642E/695K, 95D/167D/197K/598K/695K/725H, 95D/249E/433K/642E, 95D/249E/598K, 95D/598K, 99G, 99L, 99P, 99S, 99T, 105E, 105F, 105G, 105L, 105S, 105T, 122G, 122I, 122L, 122N, 122R, 122S, 139D/167D, 139D/167D/197K/433K/501K/642E, 139D/433K/598K, 141C, 167D, 167D/197K, 167D/197K/642E/695K/725H, 167D/642E, 167D/725H, 177C, 177L, 177Q, 177R, 197K/642E/725H, 197K/695K, 202G, 204L, 209G, 209M, 209N, 209V, 217G, 217R, 217T, 227M, 227S, 2391, 239L, 239W, 239Y, 247A, 247G, 249E/695K, 249E/725H, 266E, 268G, 268G/302V/451G/494S/621R, 268H, 268S, 270P, 291Q, 291Q/684P, 299E, 299F, 299G, 299K, 299M, 299P, 299R/474V, 299V, 302V, 302V/423E/451G/494S/621R, 302V/423E/621R, 302V/451G/494S, 313F, 313S, 313T, 313V, 346H, 346M, 346P, 346R, 346T, 349N, 349P, 349R, 349T, 349V, 349W, 356A, 356G, 356I, 356Q, 356R, 356V, 361G, 370D, 370T, 370Y, 374G, 377G, 377L, 394C, 394D, 394G, 394M, 394R, 404C, 404K, 404R, 404S, 404V, 423E, 423E/451G/621R/696Q, 423E/494V/525R, 423F, 423G, 423R, 423S, 423V, 426A, 426G, 426H, 426Q, 426R/798F, 426V, 426W, 427A, 427C, 427E, 427K, 427L, 427M, 427R, 427S, 427T, 427W, 433K/501K/725H, 436E, 436G, 436H, 436K, 436S, 436W, 436Y, 439F/509V/7891, 440C, 440G, 440W, 444T, 444V, 445G/446V, 445H, 446M/811I, 448G, 448K, 448Q, 448S, 448T, 448V/793P, 459F, 459L, 459R, 461A, 482A, 484V, 484V/684P, 485C, 485D, 485F, 485M, 485N, 485Q, 485S, 485T, 489S, 489W, 494V, 498M/684P, 509A, 514R, 514S, 517I, 517N, 517R, 517S, 517T, 525R/529D/621R, 533C, 533R, 543V/621R/721R, 547M, 565C, 565R, 565S, 565T, 574L, 574Q, 596A, 596V, 598K, 598K/642E, 598K/642E/695K, 598K/695K, 598K/725H, 601S, 610A, 621R, 621R/659G, 636G, 642E, 642E/692D, 659A, 659G, 659N, 659R, 659T, 662D, 662F, 662H, 662Q, 662T, 662V, 662W, 673K, 683K, 684P, 688A, 688C, 688G, 689G, 690G, 690R, 690S, 695K, 725H, 741G, 741S, 744C, 744G/793P, 744L, 744Q, 744R, 744W, 751G, 801S, 801T, 810C, 810D, 810F, 810G, 810N, 818A, 818E, 818F, 818L, 818R, 818S, 830M, 832F, 832L, and 832Y, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from H4A, H4F, H4G, H4R, H4T, H4V, H6F, H6G, H6N, H6R, H6V, S10I, S10Q, S10R, G11E, G11L, G11R, V12D, V12W, P14E, P14E/Q50H, P14E/Q50H/A56V/P598K/T725H, P14E/Q50H/P598K/Y695K, P14E/P95D, P14E/P95D/R501K/Q642E, P14E/R139D/K249E/E433K, P14E/R139D/T725H, P14E/E167D, P14E/E167D/P197K/E433K, P14E/E167D/P197K/R501K/T725H, P14E/E167D/P197K/Q642E/Y695K, P14E/E167D/K249E/E433K/P598K/Q642E, P14E/E167D/K249E/R501K/P598K/Q642E, P14E/E167D/K249E/P598K/Y695K/T725H, P14E/E167D/K249E/Q642E, P14E/E167D/Q642E, P14E/E167D/Y695K, P14E/E167D/T725H, P14E/P197K/K249E/Q642E, P14E/P197K/Q642E, P14E/K249E/E433K/T725H, P14E/K249E/P598K, P14E/K249E/Q642E, P14E/K249E/T725H, P14E/E433K/P598K, P14E/R501K, P14E/P598K, P14E/P598K/Y695K, P14E/P598K/Y695K/T725H, P14E/Q642E/T725H, P18H, P18H/P302V, P18H/P302V/L531V/T673K/A684P, P18H/P302V/L547M, P18H/F484V/T673K, P18H/F484V/T673K/A684P, P18H/S524D/L547M, P18H/L547M, P18H/A684P, H29M, H29N, H29V, H29W, S45L/Q268G/S299N/P302L/S321T/S621R/S659G, S45L/Q268G/P302L/S621R/E721R, S45L/L376F/L385F/T408A/N494V/A525R/S621R/E696Q, S45L/A423E, P48M, P48V, Q50H, Q50H/E433K/P598K, Q50H/R501K, Q50H/P598K, Q50H/P598K/Y695K, Q50H/Q642E, Y53A, Y53Q, Y53W, A76L, A76S, A76W, A76Y, K77H, K77M, K77R, K77S, A91C, A91R, A91S, A91W, P95D/E167D/P197K/P598K/Q642E/Y695K, P95D/E167D/P197K/P598K/Y695K/T725H, P95D/K249E/E433K/Q642E, P95D/K249E/P598K, P95D/P598K, D99G, D99L, D99P, D99S, D99T, A105E, A105F, A105G, A105L, A105S, A105T, P122G, P122I, P122L, P122N, P122R, P122S, R139D/E167D, R139D/E167D/P197K/E433K/R501K/Q642E, R139D/E433K/P598K, G141C, E167D, E167D/P197K, E167D/P197K/Q642E/Y695K/T725H, E167D/Q642E, E167D/T725H, W177C, W177L, W177Q, W177R, P197K/Q642E/T725H, P197K/Y695K, P202G, V204L, E209G, E209M, E209N, E209V, K217G, K217R, K217T, K227M, K227S, E2391, E239L, E239W, E239Y, D247A, D247G, K249E/Y695K, K249E/T725H, D266E, Q268G, Q268G/P302V/A451G/N494S/S621R, Q268H, Q268S, R270P, L291Q, L291Q/A684P, S299E, S299F, S299G, S299K, S299M, S299P, S299R/A474V, S299V, P302V, P302V/A423E/A451G/N494S/S621R, P302V/A423E/S621R, P302V/A451G/N494S, G313F, G313S, G313T, G313V, D346H, D346M, D346P, D346R, D346T, G349N, G349P, G349R, G349T, G349V, G349W, E356A, E356G, E356I, E356Q, E356R, E356V, L361G, L370D, L370T, L370Y, L374G, P377G, P377L, T394C, T394D, T394G, T394M, T394R, G404C, G404K, G404R, G404S, G404V, A423E, A423E/A451G/S621R/E696Q, A423E/N494V/A525R, A423F, A423G, A423R, A423S, A423V, L426A, L426G, L426H, L426Q, L426R/L798F, L426V, L426W, G427A, G427C, G427E, G427K, G427L, G427M, G427R, G427S, G427T, G427W, E433K/R501K/T725H, L436E, L436G, L436H, L436K, L436S, L436W, L436Y, Y439F/L509V/L7891, E440C, E440G, E440W, K444T, K444V, P445G/L446V, P445H, L446M/L811I, R448G, R448K, R448Q, R448S, R448T, R448V/H793P, K459F, K459L, K459R, D461A, E482A, F484V, F484V/A684P, R485C, R485D, R485F, R485M, R485N, R485Q, R485S, R485T, R489S, R489W, N494V, Q498M/A684P, L509A, K514R, K514S, K517I, K517N, K517R, K517S, K517T, A525R/E529D/S621R, E533C, E533R, Q543V/S621R/E721R, L547M, K565C, K565R, K565S, K565T, N574L, N574Q, R596A, R596V, P598K, P598K/Q642E, P598K/Q642E/Y695K, P598K/Y695K, P598K/T725H, Q601S, E610A, S621R, S621R/S659G, N636G, Q642E, Q642E/G692D, S659A, S659G, S659N, S659R, S659T, A662D, A662F, A662H, A662Q, A662T, A662V, A662W, T673K, S683K, A684P, S688A, S688C, S688G, Q689G, E690G, E690R, E690S, Y695K, T725H, D741G, D741S, A744C, A744G/H793P, A744L, A744Q, A744R, A744W, E751G, P801S, P801T, A810C, A810D, A810F, A810G, A810N, G818A, G818E, G818F, G818L, G818R, G818S, V830M, 1832F, 1832L, and I832Y, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from 4, 6, 10, 11, 14, 14/50, 14/139/725, 14/167, 14/167/642, 14/167/695, 14/598, 18/302/531/673/684, 18/524/547, 18/684, 45/268/299/302/321/621/659, 45/268/302/621/721, 48, 50/598, 77, 95/167/197/598/695/725, 99, 105, 122, 167, 167/197, 177, 209, 217, 239, 249/695, 268, 346, 349, 356, 423, 423/451/621/696, 426, 426/798, 427, 433/501/725, 436, 439/509/789, 445, 448, 448/793, 482, 484, 484/684, 514, 517, 533, 543/621/721, 565, 574, 598/695, 621, 621/659, 659, 662, 684, 688, 690, 725, 744/793, 801, 810, and 832, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from 4G, 6G, 6R, 101, 11E, 11R, 14E, 14E/50H, 14E/139D/725H, 14E/167D, 14E/167D/642E, 14E/167D/695K, 14E/598K, 18H/302V/531V/673K/684P, 18H/524D/547M, 18H/684P, 45L/268G/299N/302L/321T/621R/659G, 45L/268G/302L/621R/721R, 48V, 50H/598K, 77M, 95D/167D/197K/598K/695K/725H, 99S, 99T, 105F, 105T, 122G, 122R, 167D, 167D/197K, 177L, 209V, 217G, 2391, 249E/695K, 268S, 346P, 346R, 349P, 349R, 349V, 356V, 423E/451G/621R/696Q, 423G, 423S, 426G, 426H, 426R/798F, 426W, 427A, 427C, 427L, 427R, 433K/501K/725H, 436E, 436K, 436S, 439F/509V/7891, 445H, 448S, 448V/793P, 482A, 484V, 484V/684P, 514R, 514S, 5171, 517T, 533C, 543V/621R/721R, 565R, 574L, 598K/695K, 621R, 621R/659G, 659G, 659R, 662H, 662T, 662W, 684P, 688A, 688C, 688G, 690G, 725H, 744G/793P, 801T, 810D, 810F, 810N, and 832L, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from H4G, H6G, H6R, S10I, G11E, G11R, P14E, P14E/Q50H, P14E/R139D/T725H, P14E/E167D, P14E/E167D/Q642E, P14E/E167D/Y695K, P14E/P598K, P18H/P302V/L531V/T673K/A684P, P18H/S524D/L547M, P18H/A684P, S45L/Q268G/S299N/P302L/S321T/S621R/S659G, S45L/Q268G/P302L/S621R/E721R, P48V, Q50H/P598K, K77M, P95D/E167D/P197K/P598K/Y695K/T725H, D99S, D99T, A105F, A105T, P122G, P122R, E167D, E167D/P197K, W177L, E209V, K217G, E239I, K249E/Y695K, Q268S, D346P, D346R, G349P, G349R, G349V, E356V, A423E/A451G/S621R/E696Q, A423G, A423S, L426G, L426H, L426R/L798F, L426W, G427A, G427C, G427L, G427R, E433K/R501K/T725H, L436E, L436K, L436S, Y439F/L509V/L789I, P445H, R448S, R448V/H793P, E482A, F484V, F484V/A684P, K514R, K514S, K517I, K517T, E533C, Q543V/S621R/E721R, K565R, N574L, P598K/Y695K, S621R, S621R/S659G, S659G, S659R, A662H, A662T, A662W, A684P, S688A, S688C, S688G, E690G, T725H, A744G/H793P, P801T, A810D, A810F, A810N, and I832L, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from 11, 12, 18/547, 45/268/299/302/321/621/659, 45/268/302/621/721, 45/376/385/408/494/525/621/696, 48, 105, 122, 302, 346, 426/798, 439/509/789, 448/793, 484, 514, 525/529/621, 533, 543/621/721, 574, 690, and 744/793, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from 11E, 12W, 18H/547M, 45L/268G/299N/302L/321T/621R/659G, 45L/268G/302L/621R/721R, 45L/376F/385F/408A/494V/525R/621R/696Q, 48M, 105F, 122G, 302V, 346R, 426R/798F, 439F/509V/789I, 448V/793P, 484V, 514S, 525R/529D/621R, 533C, 543V/621R/721R, 574Q, 690G, and 744G/793P, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from G11E, V12W, P18H/L547M, S45L/Q268G/S299N/P302L/S321T/S621R/S659G, S45L/Q268G/P302L/S621R/E721R, S45L/L376F/L385F/T408A/N494V/A525R/S621R/E696Q, P48M, A105F, P122G, P302V, D346R, L426R/L798F, Y439F/L509V/L789I, R448V/H793P, F484V, K514S, A525R/E529D/S621R, E533C, Q543V/S621R/E721R, N574Q, E690G, and A744G/H793P, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from 11, 12, 18, 18/302, 18/302/547, 18/547, 45/268/299/302/321/621/659, 45/268/302/621/721, 45/376/385/408/494/525/621/696, 48, 105, 122, 291/684, 302, 346, 426/798, 439/509/789, 448/793, 484, 514, 525/529/621, 533, 547, 574, 684, 690, and 744/793, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from 11E, 12W, 18H, 18H/302V, 18H/302V/547M, 18H/547M, 45L/268G/299N/302L/321T/621R/659G, 45L/268G/302L/621R/721R, 45L/376F/385F/408A/494V/525R/621R/696Q, 48M, 105F, 122G, 291Q/684P, 302V, 346R, 426R/798F, 439F/509V/789I, 448V/793P, 484V, 514S, 525R/529D/621R, 533C, 547M, 574Q, 684P, 690G, and 744G/793P, or combinations thereof, wherein the amino acid positions are relative to the reference sequence of SEQ ID NO: 2994.

In some embodiments, the present invention provides an engineered polynucleotide encoding an engineered DNA polymerase comprising one or more substitutions or substitution sets selected from G11E, V12W, P18H, P18H/P302V, P18H/P302V/L547M, P18H/L547M, S45L/Q268G/S299N/P302L/S321T/S621R/S659G, S45L/Q268G/P302L/S621R/E721R, S45L/L376F/L385F/T408A/N494V/A525R/S621R/E696Q, P48M, A105F, P122G, L291Q/A684P, P302V, D346R, L426R/L798F, Y439F/L509V/L789I, R448V/H793P, F484V, K514S, A525R/E529D/S621R, E533C, L547M, N574Q, A684P, E690G, and A744G/H793P, or combinations thereof, wherein the amino acid positions are relative to the reference sequence of SEQ ID NO: 2994.

The present invention also provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 3796, or to a reference sequence of SEQ ID NO: 3796, wherein the polypeptide sequence comprises one or more substitutions relative to the reference sequence comprising residues 12 to 841 of SEQ ID NO: 3796, or to the reference sequence of SEQ ID NO: 3796.

In some embodiments, the present invention provides an engineered polynucleotide encoding an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 3796 or to the reference sequence of SEQ ID NO: 3796.

In some embodiments, the present invention provides an engineered polynucleotide encoding an engineered DNA polymerase comprising one or more substitutions or substitution sets selected from 1/2/149, 15/167, 18, 18/105/139/197/249/426/598, 18/105/139/197/291/299/423/426/448/529/547/725, 18/105/249/284/302/423/426/501/673/684/798, 18/105/321/547, 18/139/197/249/299/302/433/547/642/659/695/744, 18/139/197/426/598, 18/139/197/642, 18/139/249/423/433/501/547/725, 18/197/249/291/484, 18/197/321/426/448/451/659/673/744, 18/197/321/433/484, 18/249, 18/249/299/302/423/529/533/642/659/673, 18/249/302/423/484/547/695, 18/249/448/484/673, 18/249/673, 18/291/302/423/684, 18/291/302/433, 18/291/302/525/547/659/684/695, 18/291/302/684, 18/291/423/525/529, 18/291/659, 18/291/695, 18/299/302/423/426/525/529, 18/299/451/525/529/695, 18/302/423/529, 18/302/433/547/684, 18/302/525/529/547, 18/302/529/659/695, 18/321/423, 18/321/484/547/725, 18/423, 18/423/529/695, 18/433, 18/433/673/725, 18/484, 18/598/725, 32, 38/463, 64, 105/139/291/302/423/484/547/725, 105/139/448/501/525/529/642/659, 105/197/249/423/426/547/642/673, 105/321/433/744, 105/321/501, 137, 139/249/291/321/484/695/725, 139/249/291/451/744, 139/249/426/451/501/673/744, 149, 160, 164/172, 167, 172, 197/249/327/423/673/690, 197/302/391/547/598, 197/525/529/659/744, 197/547, 197/684/725, 200/577, 245/423, 247/423/448, 249/321/673/798, 249/423/426/673, 249/448/451/547/659/725, 249/725/744/798, 253, 256, 256/455, 271, 281, 291/302/423, 291/302/529, 291/321/423/725, 291/423/525, 291/423/529/533/598/642/725/798, 291/484/598/642/673/744/798, 291/525, 300, 302, 302/364/376/383/386/408/451/484/598, 302/423/433/529/695, 302/423/525/529, 302/423/547/684/695, 302/684/695, 311, 314, 316, 321/448/529/673, 323, 330, 334/431, 347, 348, 352/363, 353, 354, 358, 361, 363, 368, 372, 376, 376/699, 383, 389, 392, 394, 411, 414, 422, 423/424/547, 423/433/484/529/533/642, 423/433/487/525/547, 423/484/673, 423/529/684, 423/547/598/642, 430, 431, 433/525/529/659/684/695, 436/743, 448/451/529/725, 451/598/725, 455, 455/518, 462, 463, 464, 468, 469, 472, 474, 474/821, 476, 479, 480, 484, 489, 491, 492, 498, 501, 504/548, 505, 510, 519/720, 521, 524, 525, 525/533, 529, 531, 532, 533, 535, 540, 546, 547, 550, 553, 555, 555/572, 557, 558, 559, 566, 568, 570, 571, 572, 573, 574, 575, 576, 577, 583, 588, 590, 590/652, 592, 597, 600, 601, 612, 615, 618, 620, 622, 623, 625, 628, 643, 654, 655, 660, 664, 675, 677, 682, 685, 690, 691/692, 692, 692/766, 697, 703, 705, 708, 711, 715, 720, 726, 728, 730, 732, 738, 743, 746, 752, 753, 756, 766, 769, 770, 779, 793, 800, 802, 803, 805, 813, 819, 825, 827, and 829, or combinations thereof, wherein the amino acid positions are relative to the reference sequence of SEQ ID NO: 3796.

In some embodiments, the present invention provides an engineered polynucleotide encoding an engineered DNA polymerase comprising one or more substitutions or substitution sets selected from 1H/2M/149G, 15M/167V, 18H, 18H/105F/139D/197K/249E/426R/598K, 18H/105F/139D/197K/291Q/299N/423E/426R/448V/529D/547M/725H, 18H/105F/249E/284C/302V/423E/426R/501K/673K/684P/798F, 18H/105F/321T/547M, 18H/139D/197K/249E/299N/302V/433K/547M/642E/659G/695K/744G, 18H/139D/197K/426R/598K, 18H/139D/197K/642E, 18H/139D/249E/423E/433K/501K/547M/725H, 18H/197K/249E/291Q/484V, 18H/197K/321T/426R/448V/451G/659G/673K/744G, 18H/197K/321T/433K/484V, 18H/249E, 18H/249E/299N/302V/423E/529D/533C/642E/659G/673K, 18H/249E/302V/423E/484V/547M/695K, 18H/249E/448V/484V/673K, 18H/249E/673K, 18H/291Q/302V/423E/684P, 18H/291Q/302V/433K, 18H/291Q/302V/525R/547M/659G/684P/695K, 18H/291Q/302V/684P, 18H/291Q/423E/525R/529D, 18H/291Q/659G, 18H/291Q/695K, 18H/299N/302V/423E/426R/525R/529D, 18H/299N/451G/525R/529D/695K, 18H/302V/423E/529D, 18H/302V/433K/547M/684P, 18H/302V/525R/529D/547M, 18H/302V/529D/659G/695K, 18H/321T/423E, 18H/321T/484V/547M/725H, 18H/423E, 18H/423E/529D/695K, 18H/433K, 18H/433K/673K/725H, 18H/484V, 18H/598K/725H, 32G, 32L, 32S, 38M/463G, 64G, 105F/139D/291Q/302V/423E/484V/547M/725H, 105F/139D/448V/501K/525R/529D/642E/659G, 105F/197K/249E/423E/426R/547M/642E/673K, 105F/321T/433K/744G, 105F/321T/501K, 137L, 139D/249E/291Q/321T/484V/695K/725H, 139D/249E/291Q/451G/744G, 139D/249E/426R/451G/501K/673K/744G, 149G, 149P, 149V, 160G, 160L, 164V/172F, 167A, 167G, 172H, 172V, 172W, 197K/249E/327L/423E/673K/690G, 197K/302V/391S/547M/598K, 197K/525R/529D/659G/744G, 197K/547M, 197K/684P/725H, 200S/577G, 2451/423L, 247Y/423E/448V, 249E/321T/673K/798F, 249E/423E/426R/673K, 249E/448V/451G/547M/659G/725H, 249E/725H/744G/798F, 253C, 253I, 253M, 256F, 256S, 256S/455F, 256V, 271L, 281V, 291Q/302V/423E, 291Q/302V/529D, 291Q/321T/423E/725H, 291Q/423E/525R, 291Q/423E/529D/533C/598K/642E/725H/798F, 291Q/484V/598K/642E/673K/744G/798F, 291Q/525R, 300G, 302G, 302M, 302S, 302V/364N/376F/383K/386G/408A/451G/484V/598K, 302V/423E/433K/529D/695K, 302V/423E/525R/529D, 302V/423E/547M/684P/695K, 302V/684P/695K, 311C, 311G, 311H, 311K, 314L, 314M, 316N, 321T/448V/529D/673K, 323W, 330M, 330T, 330V, 334T/431C, 347L, 347R, 348A, 352S/363L, 3531, 353T, 354G, 354M, 358E, 358L, 361C, 361R, 361W, 363L, 363T, 363V, 368A, 368L, 368R, 368W, 372K, 372L, 372R, 372S, 372W, 3761, 376K, 376W, 376W/699L, 383I, 383R, 383S, 389G, 389R, 389S, 392G, 392R, 392T, 392V, 394D, 411E, 414S, 422A, 422G, 422L, 422Q, 422R, 423E/424K/547M, 423E/433K/484V/529D/533C/642E, 423E/433K/487V/525R/547M, 423E/484V/673K, 423E/529D/684P, 423E/547M/598K/642E, 430R, 431D, 431Q, 431S, 431T, 431V, 433K/525R/529D/659G/684P/695K, 436F/743K, 448V/451G/529D/725H, 451G/598K/725H, 455E, 455G, 455L, 455P/518I, 455Q, 455S, 455V, 455W, 462A, 462G, 462L, 462Q, 462R, 462S, 462W, 463T, 464H, 464K, 464V, 468A, 468G, 468T, 468W, 469R, 472E, 472G, 472L, 472S, 472T, 474E, 474L/821S, 474R, 474S, 476S, 479W, 480G, 484E, 484G, 484L, 484Q, 484T, 484W, 489H, 489L, 489Q, 491W, 492K, 492Y, 498C, 498D, 501A, 501L, 501S, 504S/548F, 505V, 510G, 510S, 510T, 519D/720S, 521W, 524K, 525G, 525Q, 525R, 525R/533C, 525S, 525V, 529S, 531A, 532L, 533G, 533L, 533N, 533R, 533W, 535N, 535S, 540C, 540S, 540T, 546C, 546R, 547M, 550G, 550I, 550P, 550Y, 553L, 553S, 555A, 555L, 555T/572H, 555V, 557L, 558Q, 559L, 559R, 566G, 568C, 568K, 570S, 571Y, 572N, 572T, 573T, 574M, 575C, 575R, 576K, 576W, 577G, 577H, 577L, 577S, 577W, 583M, 583Q, 588F, 588G, 588V, 590F, 590I/652T, 592S, 597G, 597S, 597V, 600A, 600K, 600R, 600S, 601F, 601L, 612E, 612S, 612T, 612V, 615S, 615Y, 618C, 620L, 620M, 620T, 622G, 622L, 622S, 623G, 623L, 623V, 623W, 625I, 625T, 628M, 628T, 643G, 643L, 643M, 643V, 643Y, 654F, 654G, 654K, 655A, 655F, 655L, 655V, 660E, 660G, 660R, 660W, 664L, 664M, 675G, 675L, 675M, 675T, 677A, 682K, 685R, 685S, 690G, 690R, 690T, 690V, 690W, 691F/692H, 692L, 692S/766T, 692V, 697G, 697Q, 697S, 703F, 703G, 703K, 703P, 703R, 703V, 705F, 705G, 705M, 705V, 708R, 711S, 711V, 711W, 715Q, 715V, 720G, 726T, 728A, 728F, 728Q, 728R, 728S, 728V, 728W, 730R, 730V, 732E, 738G, 738M, 738W, 743A, 743G, 743Q, 743R, 743W, 746-, 752L, 752V, 753G, 753K, 753L, 753R, 756H, 756R, 756T, 766G, 766V, 769A, 769C, 769G, 769M, 769V, 770R, 779R, 793T, 800G, 800I, 800M, 800P, 802C, 802G, 802R, 803S, 805F, 805G, 805I, 805M, 805Q, 805T, 813G, 819A, 819L, 819R, 819S, 819W, 825G, 825S, 825Y, 827G, 827V, 827W, 829L, 829P, and 829W, or combinations thereof, wherein the amino acid positions are relative to the reference sequence of SEQ ID NO: 3796.

In some embodiments, the present invention provides an engineered polynucleotide encoding an engineered DNA polymerase comprising one or more substitutions or substitution sets selected from M1H/H2M/A149G, V15M/E167V, P18H, P18H/A105F/R139D/P197K/K249E/L426R/P598K, P18H/A105F/R139D/P197K/L291Q/S299N/A423E/L426R/R448V/E529D/L547M/T725H, P18H/A105F/K249E/R284C/L302V/A423E/L426R/R501K/T673K/A684P/L798F, P18H/A105F/S321T/L547M, P18H/R139D/P197K/K249E/S299N/L302V/E433K/L547M/Q642E/S659G/Y695K/A744G, P18H/R139D/P197K/L426R/P598K, P18H/R139D/P197K/Q642E, P18H/R139D/K249E/A423E/E433K/R501K/L547M/T725H, P18H/P197K/K249E/L291Q/F484V, P18H/P197K/S321T/L426R/R448V/A451G/S659G/T673K/A744G, P18H/P197K/S321T/E433K/F484V, P18H/K249E, P18H/K249E/S299N/L302V/A423E/E529D/E533C/Q642E/S659G/T673K, P18H/K249E/L302V/A423E/F484V/L547M/Y695K, P18H/K249E/R448V/F484V/T673K, P18H/K249E/T673K, P18H/L291Q/L302V/A423E/A684P, P18H/L291Q/L302V/E433K, P18H/L291Q/L302V/A525R/L547M/S659G/A684P/Y695K, P18H/L291Q/L302V/A684P, P18H/L291Q/A423E/A525R/E529D, P18H/L291Q/S659G, P18H/L291Q/Y695K, P18H/S299N/L302V/A423E/L426R/A525R/E529D, P18H/S299N/A451G/A525R/E529D/Y695K, P18H/L302V/A423E/E529D, P18H/L302V/E433K/L547M/A684P, P18H/L302V/A525R/E529D/L547M, P18H/L302V/E529D/S659G/Y695K, P18H/S321T/A423E, P18H/S321T/F484V/L547M/T725H, P18H/A423E, P18H/A423E/E529D/Y695K, P18H/E433K, P18H/E433K/T673K/T725H, P18H/F484V, P18H/P598K/T725H, Y32G, Y32L, Y32S, L38M/A463G, K64G, A105F/R139D/L291Q/L302V/A423E/F484V/L547M/T725H, A105F/R139D/R448V/R501K/A525R/E529D/Q642E/S659G, A105F/P197K/K249E/A423E/L426R/L547M/Q642E/T673K, A105F/S321T/E433K/A744G, A105F/S321T/R501K, A137L, R139D/K249E/L291Q/S321T/F484V/Y695K/T725H, R139D/K249E/L291Q/A451G/A744G, R139D/K249E/L426R/A451G/R501K/T673K/A744G, A149G, A149P, A149V, R160G, R160L, L164V/T172F, E167A, E167G, T172H, T172V, T172W, P197K/K249E/W327L/A423E/T673K/E690G, P197K/L302V/P391S/L547M/P598K, P197K/A525R/E529D/S659G/A744G, P197K/L547M, P197K/A684P/T725H, N200S/A577G, L245I/A423L, D247Y/A423E/R448V, K249E/S321T/T673K/L798F, K249E/A423E/L426R/T673K, K249E/R448V/A451G/L547M/S659G/T725H, K249E/T725H/A744G/L798F, E253C, E253I, E253M, R256F, R256S, R256S/A455F, R256V, R271L, F281V, L291Q/L302V/A423E, L291Q/L302V/E529D, L291Q/S321T/A423E/T725H, L291Q/A423E/A525R, L291Q/A423E/E529D/E533C/P598K/Q642E/T725H/L798F, L291Q/F484V/P598K/Q642E/T673K/A744G/L798F, L291Q/A525R, P300G, L302G, L302M, L302S, L302V/D364N/L376F/M383K/A386G/T408A/A451G/F484V/P598K, L302V/A423E/E433K/E529D/Y695K, L302V/A423E/A525R/E529D, L302V/A423E/L547M/A684P/Y695K, L302V/A684P/Y695K, P311C, P311G, P311H, P311K, A314L, A314M, L316N, S321T/R448V/E529D/T673K, P323W, L330M, L330T, L330V, A334T/G431C, P347L, P347R, H348A, R352S/K363L, G353I, G353T, L354G, L354M, R358E, R358L, L361C, L361R, L361W, K363L, K363T, K363V, V368A, V368L, V368R, V368W, E372K, E372L, E372R, E372S, E372W, L376I, L376K, L376W, L376W/Q699L, M383I, M383R, M383S, L389G, L389R, L389S, S392G, S392R, S392T, S392V, T394D, A411E, R414S, H422A, H422G, H422L, H422Q, H422R, A423E/N424K/L547M, A423E/E433K/F484V/E529D/E533C/Q642E, A423E/E433K/A487V/A525R/L547M, A423E/F484V/T673K, A423E/E529D/A684P, A423E/L547M/P598K/Q642E, Q430R, G431D, G431Q, G431S, G431T, G431V, E433K/A525R/E529D/S659G/A684P/Y695K, L436F/N743K, R448V/A451G/E529D/T725H, A451G/P598K/T725H, A455E, A455G, A455L, A455P/T518I, A455Q, A455S, A455V, A455W, V462A, V462G, V462L, V462Q, V462R, V462S, V462W, A463T, Y464H, Y464K, Y464V, L468A, L468G, L468T, L468W, S469R, V472E, V472G, V472L, V472S, V472T, A474E, A474L/P821S, A474R, A474S, M476S, L479W, E480G, F484E, F484G, F484L, F484Q, F484T, F484W, R489H, R489L, R489Q, F491W, R492K, R492Y, Q498C, Q498D, R501A, R501L, R501S, F504S/A548F, D505V, P510G, P510S, P510T, G519D/L720S, R521W, S524K, A525G, A525Q, A525R, A525R/E533C, A525S, A525V, E529S, L531A, R532L, E533G, E533L, E533N, E533R, E533W, H535N, H535S, K540C, K540S, K540T, E546C, E546R, L547M, L550G, L550I, L550P, L550Y, T553L, T553S, 1555A, 1555L, 1555T/R572H, 1555V, R557L, L558Q, P559L, P559R, T566G, R568C, R568K, H570S, T571Y, R572N, R572T, F573T, N574M, Q575C, Q575R, T576K, T576W, A577G, A577H, A577L, A577S, A577W, L583M, L583Q, P588F, P588G, P588V, L590F, L590I/A652T, N592S, T597G, T597S, T597V, G600A, G600K, G600R, G600S, Q601F, Q601L, G612E, G612S, G612T, G612V, L615S, L615Y, L618C, Y620L, Y620M, Y620T, Q622G, Q622L, Q622S, 1623G, I623L, 1623V, 1623W, L625I, L625T, L628M, L628T, E643G, E643L, E643M, E643V, E643Y, W654F, W654G, W654K, M655A, M655F, M655L, M655V, A660E, A660G, A660R, A660W, D664L, D664M, N675G, N675L, N675M, N675T, G677A, M682K, F685R, F685S, E690G, E690R, E690T, E690V, E690W, L691F/G692H, G692L, G692S/A766T, G692V, E697G, E697Q, E697S, Q703F, Q703G, Q703K, Q703P, Q703R, Q703V, Y705F, Y705G, Y705M, Y705V, S708R, K711S, K711V, K711W, W715Q, W715V, L720G, R726T, Y728A, Y728F, Y728Q, Y728R, Y728S, Y728V, Y728W, E730R, E730V, L732E, Y738G, Y738M, Y738W, N743A, N743G, N743Q, N743R, N743W, V746-, A752L, A752V, A753G, A753K, A753L, A753R, M756H, M756R, M756T, A766G, A766V, L769A, L769C, L769G, L769M, L769V, M770R, P779R, H793T, A800G, A800I, A800M, A800P, K802C, K802G, K802R, E803S, A805F, A805G, A805I, A805M, A805Q, A805T, K813G, V819A, V819L, V819R, V819S, V819W, P825G, P825S, P825Y, E827G, E827V, E827W, E829L, E829P, and E829W, or combinations thereof, wherein the amino acid positions are relative to the reference sequence of SEQ ID NO: 3796.

The present invention also provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 4182, or to a reference sequence of SEQ ID NO: 4182, wherein the polypeptide sequence comprises one or more substitutions relative to the reference sequence comprising residues 12 to 841 of SEQ ID NO: 4182, or to the reference sequence of SEQ ID NO: 4182.

In some embodiments, the present invention provides an engineered polynucleotide encoding an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 4182 or to the reference sequence of SEQ ID NO: 4182.

In some embodiments, the present invention provides an engineered polynucleotide encoding an engineered DNA polymerase comprising one or more substitutions or substitution sets selected from 18/149/300/423/424/743/769, 18/256/423/424/455/673, 18/372/424/533/546/600/673/769, 18/424,149/256/300/302/424/484/769, 149/256/300/372/424/546, 149/424, 256/300/363/423/424/546/673/690, 424, 424/533, and 424/723, or combinations thereof, wherein the amino acid positions are relative to the reference sequence of SEQ ID NO: 4182.

In some embodiments, the present invention provides an engineered polynucleotide encoding an engineered DNA polymerase comprising one or more substitutions or substitution sets selected from 18H/149G/300G/423A/424N/743A/769C, 18H/256S/423A/424N/455S/673K, 18H/372S/424N/533W/546C/600A/673K/769C, 18H/424N, 149G/256S/300G/302G/424N/484V/769C, 149G/256S/300G/372S/424N/546C, 149G/424N, 256S/300G/363L/423A/424N/546C/673K/690V, 424N, 424N/533W, and 424N/723D, or combinations thereof, wherein the amino acid positions are relative to the reference sequence of SEQ ID NO: 4182.

In some embodiments, the present invention provides an engineered polynucleotide encoding an engineered DNA polymerase comprising one or more substitutions or substitution sets selected from P18H/A149G/P300G/E423A/K424N/N743A/L769C, P18H/R256S/E423A/K424N/A455S/T673K, P18H/E372S/K424N/E533W/E546C/G600A/T673K/L769C, P18H/K424N, A149G/R256S/P300G/L302G/K424N/F484V/L769C, A149G/R256S/P300G/E372S/K424N/E546C, A149G/K424N, R256S/P300G/K363L/E423A/K424N/E546C/T673K/E690V, K424N, K424N/E533W, and K424N/G723D, or combinations thereof, wherein the amino acid positions are relative to the reference sequence of SEQ ID NO: 4182.

The present invention also provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 5094, or to a reference sequence of SEQ ID NO: 5094, wherein the polypeptide sequence comprises one or more substitutions relative to the reference sequence comprising residues 12 to 841 of SEQ ID NO: 5094, or to the reference sequence of SEQ ID NO: 5094.

In some embodiments, the present invention provides an engineered polynucleotide encoding an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5094 or to the reference sequence of SEQ ID NO: 5094.

In some embodiments, the present invention provides an engineered polynucleotide encoding an engineered DNA polymerase comprising one or more substitutions or substitution sets selected from 9/689, 9/691, 21/695/700, 45, 45/268, 45/268/302/423, 45/268/302/467/621/748/749, 45/268/302/469, 45/268/302/469/547/557/721/749, 45/268/302/475/489, 45/268/302/547/557/621/748/749, 45/268/423/467/748, 45/268/423/469/721, 45/268/423/475/547/721/749, 45/268/469, 45/268/475, 45/268/475/489, 45/268/475/621, 45/268/489/621/749, 45/268/621/721, 45/302, 45/302/423/469/475, 45/302/423/469/621, 45/302/423/469/621/721/748/749, 45/302/423/547/621/721, 45/302/469/547/721, 45/302/475/621/748/749, 45/302/621, 45/423, 45/423/467, 45/423/467/469/547/721/749, 45/423/467/469/749, 45/423/467/475/547/621/721/749, 45/423/467/621, 45/423/469/475, 45/423/469/475/621/721/749, 45/423/469/489/621, 45/423/475/547/721, 45/423/475/557/621/749, 45/423/489, 45/423/489/557, 45/467/469/557, 45/467/621, 45/467/748, 45/469/721, 45/469/749, 45/475/489/547/749, 45/547/749, 45/557, 45/721, 83/700/744, 95/268/302/423/489/547/557/721/748/749, 116, 228/708, 268, 268/302/423/557/721, 268/302/721, 268/423, 268/423/467/547, 268/423/469, 268/423/469/475, 268/467, 268/467/469/475, 268/467/748, 268/475/547, 268/475/557/721/749, 268/489/547, 296/433, 302, 302/423/467/621/748, 302/423/469/489/721/748/749, 302/423/475/489/547/621, 302/423/547/721, 302/423/557, 302/467/469/547/557, 302/467/489/748/749, 302/467/621, 302/467/721/748, 302/469, 302/469/489/621/721/748, 302/489, 302/489/547/621/699/749, 302/557/721, 302/621, 302/721, 302/749, 306, 306/459, 314/538, 321, 322, 329/359/611/700/744, 332, 335, 339, 341/532, 344, 345, 351, 354/359/408/598, 359, 359/408/412, 359/408/412/611/614/744, 359/408/412/611/708/744, 359/408/412/611/744/789, 359/408/412/700/744, 359/408/412/744, 359/408/433/614/708, 359/408/598/700, 359/408/611/614/744, 359/408/695/700/744, 359/408/695/744, 359/412, 359/412/433/436/598/611/614, 359/412/433/611/614/708, 359/412/433/611/614/744/789, 359/412/436/598/611/695/744, 359/412/469/611/695/708/744, 359/412/598, 359/412/598/611/695/700/708/744, 359/412/598/611/744, 359/412/695/700/744, 359/412/695/708/744, 359/433/611/614/700, 359/433/611/695/700/789, 359/436/611/708/744, 359/469/611, 359/469/611/614, 359/469/611/700, 359/598/611/614/744, 359/598/614/695/708/744, 359/598/700/744, 359/598/744/789, 359/611/614/695/700/744, 359/611/614/744, 359/611/700/744, 359/614, 359/695/700/789, 359/700, 359/744, 359/789, 364, 373, 378, 385, 386, 387/630, 393, 397, 399, 404, 408, 408/412, 408/412/433/436/611/614/744, 408/412/433/598/611/700/708/744, 408/412/436/598/708, 408/412/436/611/614, 408/412/469/475/611/695/708/744, 408/412/598/611/614/708, 408/412/598/700/744, 408/412/611/695/708, 408/412/708/744, 408/433/700/708/744, 408/436/598/611/708/744, 408/436/695/708/744, 408/598/611, 408/598/611/614/700, 408/598/695/700, 408/611/614/695/708, 408/611/614/708/744, 408/611/614/744, 408/611/744, 408/700, 408/744, 409, 412, 412/433/598/611/744, 412/433/611/744, 412/436/611/614/744/789, 412/436/744, 412/469/611/695/744, 412/469/611/700/744, 412/598/611/744, 412/611/614, 412/611/614/708/744/789, 412/611/789, 412/614/708, 412/689, 412/695/789, 412/708/744, 413, 417, 423, 423/467/469, 423/467/489/621/721, 423/467/547/557/621/699/721, 423/467/621/721/749, 423/467/721, 423/469, 423/469/475/489/721/748/749, 423/469/489/557/621/721/749, 423/469/547/557/621/749, 423/469/547/557/749, 423/469/621, 423/469/721, 423/489, 423/489/621, 423/598/700/708/744, 423/721, 423/748, 424, 425, 426, 433, 433/436/598/700/744, 433/436/598/744, 433/436/611/614, 433/469/611, 433/611/695/700/744, 433/744/789, 436, 436/611/614, 436/611/614/695, 436/700/744, 436/708/744, 439, 443, 446, 448, 449, 467/469, 467/489/547/621/748, 467/547/721, 467/621, 467/721, 467/721/748, 467/748, 467/749, 469, 469/471, 469/473, 469/475/621/721/748, 469/475/749, 469/614/695, 469/614/695/744, 469/621, 469/621/748, 469/744, 469/748, 475, 483, 485/791, 487, 489, 506, 507, 509, 511, 514, 517, 520, 532, 533/542, 538, 539, 539/571, 542, 546/699, 547, 547/621/721/748/749, 549, 554, 557, 557/621, 580, 584, 587, 596, 598, 598/611, 598/611/614, 598/611/708, 598/614/700/744, 598/708, 602, 602/724, 611, 611/614, 611/614/708/744/789, 611/744, 614, 614/700/708/744/789, 614/744, 614/789, 617, 621, 621/639/748/749, 621/745, 621/748, 621/749, 630, 652, 689, 691, 695, 695/708/744, 695/744, 699, 700, 700/744, 700/767, 708, 714, 721, 721/749, 723, 725, 741, 744, 745, 748, 748/749, 767, 777, 780, 785, 785/809, 787, 789, 791, 795, 798, 799, 811, 817, 821, 836, 838, and 839, or combinations thereof, wherein the amino acid positions are relative to the reference sequence of SEQ ID NO: 5094.

In some embodiments, the present invention provides an engineered polynucleotide encoding an engineered DNA polymerase comprising one or more substitutions or substitution sets selected from 9D/691S, 9V/689G, 21S/695L/700G, 45R, 45R/268R, 45R/268R/302P/423A, 45R/268R/302P/467A/621S/748S/749V, 45R/268R/302P/469H, 45R/268R/302P/469H/547L/557P/721E/749V, 45R/268R/302P/475E/489H, 45R/268R/302P/547L/557P/621S/748S/749V, 45R/268R/423A/467A/748S, 45R/268R/423A/469H/721E, 45R/268R/423A/475E/547L/721E/749V, 45R/268R/469H, 45R/268R/475E, 45R/268R/475E/489H, 45R/268R/475E/621S, 45R/268R/489H/621S/749V, 45R/268R/621S/721E, 45R/302P, 45R/302P/423A/469H/475E, 45R/302P/423A/469H/621S, 45R/302P/423A/469H/621S/721E/748S/749V, 45R/302P/423A/547L/621S/721E, 45R/302P/469H/547L/721E, 45R/302P/475E/621S/748S/749V, 45R/302P/621S, 45R/423A, 45R/423A/467A, 45R/423A/467A/469H/547L/721E/749V, 45R/423A/467A/469H/749V, 45R/423A/467A/475E/547L/621S/721E/749V, 45R/423A/467A/621S, 45R/423A/469H/475E, 45R/423A/469H/475E/621S/721E/749V, 45R/423A/469H/489H/621S, 45R/423A/475E/547L/721E, 45R/423A/475E/557P/621S/749V, 45R/423A/489H, 45R/423A/489H/557P, 45R/467A/469H/557P, 45R/467A/621S, 45R/467A/748S, 45R/469H/721E, 45R/469H/749V, 45R/475E/489H/547L/749V, 95S/268R/302P/423A/489H/547L/557P/721E/748S/749V, 116P, 228D/708E, 268R, 268R/302P/423A/557P/721E, 268R/302P/721E, 268R/423A, 268R/423A/467A/547L, 268R/423A/469H, 268R/423A/469H/475E, 268R/467A, 268R/467A/469H/475E, 268R/467A/748S, 268R/475E/547L, 268R/475E/557P/721E/749V, 268R/489H/547L, 296H/433L, 302P, 302P/423A/467A/621S/748S, 302P/423A/469H/489H/721E/748S/749V, 302P/423A/475E/489H/547L/621S, 302P/423A/547L/721E, 302P/423A/557P, 302P/467A/469H/547L/557P, 302P/467A/489H/748S/749V, 302P/467A/621S, 302P/467A/721E/748S, 302P/469H, 302P/469H/489H/621S/721E/748S, 302P/489H, 302P/489H/547L/621S/699A/749V, 302P/557P/721E, 302P/621S, 302P/721E, 302P/749V, 306E, 306L, 306M, 306N, 306P, 306R, 306S, 306T, 306T/459E, 306V, 314S/538L, 321A, 321I, 321W, 322A, 322C, 322D, 322G, 322L, 322M, 322P, 322S, 322T, 329K/359T/611P/700G/744S, 332C, 332D, 332I, 332L, 332M, 332Q, 332R, 332T, 332V, 335C, 335G, 335S, 339A, 339H, 339L, 339P, 339S, 339V, 341M/532L, 344G, 344P, 344R, 344S, 344V, 345G, 345K, 345L, 351M, 351V, 354I/359T/408W/598F, 359T, 359T/408W/412G, 359T/408W/412G/611K/614A/744S, 359T/408W/412G/611K/744S/789D, 359T/408W/412G/611P/708G/744S, 359T/408W/412G/700G/744G, 359T/408W/412G/744S, 359T/408W/433A/614A/708G, 359T/408W/598F/700G, 359T/408W/611K/614A/744G, 359T/408W/695L/700G/744G, 359T/408W/695L/744G, 359T/412G, 359T/412G/433A/436R/598F/611P/614A, 359T/412G/433A/611P/614A/708G, 359T/412G/433A/611P/614A/744S/789D, 359T/412G/436R/598F/611P/695L/744G, 359T/412G/469L/611P/695L/708G/744S, 359T/412G/598F, 359T/412G/598F/611K/744S, 359T/412G/598F/611P/695L/700G/708G/744S, 359T/412G/695L/700G/744G, 359T/412G/695L/708G/744G, 359T/433A/611P/614A/700G, 359T/433A/611P/695L/700G/789D, 359T/436R/611P/708G/744G, 359T/469L/611K, 359T/469L/611P/614A, 359T/469L/611P/700G, 359T/598F/611P/614A/744S, 359T/598F/614A/695L/708G/744S, 359T/598F/700G/744G, 359T/598F/744S/789D, 359T/611K/614A/744G, 359T/611P/614A/695L/700G/744G, 359T/611P/700G/744S, 359T/614A, 359T/695L/700G/789D, 359T/700G, 359T/744S, 359T/789D, 364G, 364P, 364R, 364T, 364V, 373F, 373K, 373P, 373S, 373V, 378C, 378F, 378G, 378L, 378R, 378Y, 385A, 385E, 385G, 385P, 385R, 385V, 386C, 386G, 386L, 386Q, 386S, 386V, 386W, 387C/630E, 393A, 393E, 393G, 393P, 393R, 393Y, 397A, 397D, 397G, 397L, 397N, 397P, 397R, 397V, 399E, 399G, 399L, 399P, 399Q, 399R, 399S, 399W, 404F, 404I, 404K, 404L, 404P, 404S, 404T, 404W, 408A, 408L, 408N, 408P, 408Q, 408R, 408W, 408W/412G, 408W/412G/433A/436R/611P/614A/744G, 408W/412G/433A/598F/611P/700G/708G/744S, 408W/412G/436R/598F/708G, 408W/412G/436R/611P/614A, 408W/412G/469L/475Q/611P/695L/708G/744S, 408W/412G/598F/611P/614A/708G, 408W/412G/598F/700G/744S, 408W/412G/611P/695L/708G, 408W/412G/708G/744G, 408W/433A/700G/708G/744S, 408W/436R/598F/611P/708G/744S, 408W/598F/695L/700G, 408W/611K/614A/744G, 408W/611P/614A/695L/708G, 408W/611P/614A/708G/744G, 408W/611P/744S, 408W/700G, 408W/744G, 408W/744S, 409G, 412F, 412G, 412G/433A/598F/611P/744G, 412G/433A/611P/744S, 412G/436R/611K/614A/744S/789D, 412G/436R/744G, 412G/469L/611P/695L/744G, 412G/469L/611P/700G/744S, 412G/598F/611P/744S, 412G/611K/614A, 412G/611P/614A/708G/744S/789D, 412G/611P/789D, 412G/614A/708G, 412G/695L/789D, 412G/708G/744G, 412K, 412L, 412P, 412Q, 412R, 412T/689V, 413E, 413G, 413H, 413L, 413R, 413S, 413T, 413W, 417A, 417G, 417Q, 417S, 423A, 423A/467A/469H, 423A/467A/489H/621S/721E, 423A/467A/547L/557P/621S/699A/721E, 423A/467A/621S/721E/749V, 423A/467A/721E, 423A/469H, 423A/469H/475E/489H/721E/748S/749V, 423A/469H/489H/557P/621S/721E/749V, 423A/469H/547L/557P/621S/749V, 423A/469H/547L/557P/749V, 423A/469H/621S, 423A/469H/721E, 423A/489H, 423A/489H/621S, 423A/721E, 423A/748S, 423G/598F/700G/708G/744S, 424E, 424G, 424L, 424T, 425C, 425S, 425T, 425Y, 426G, 426R, 426S, 426T, 426V, 433A, 433A/436R/598F/700G/744G, 433A/436R/598F/744G, 433A/436R/611P/614A, 433A/469L/611P, 433A/611P/695L/700G/744G, 433A/744S/789D, 433G, 433Q, 433R, 433S, 433W, 436A, 436G, 436Q, 436R, 436R/611K/614A, 436R/611P/614A/695L, 436R/700G/744G, 436R/708G/744G, 436S, 436T, 436V, 436W, 439A, 439E, 439L, 439S, 439W, 443C, 443T, 446S, 448E, 448L, 448W, 449G, 449W, 467A/469H, 467A/489H/547L/621S/748S, 467A/547L/721E, 467A/621S, 467A/721E, 467A/721E/748S, 467A/748S, 467A/749V, 467C/469H, 467H/469H, 467M/469H, 467R/469H, 467T/469H, 467V/469H, 467W/469H, 469E, 469H, 469H/471A, 469H/471F, 469H/471H, 469H/471L, 469H/471M, 469H/471Q, 469H/471R, 469H/473F, 469H/473W, 469H/475E/621S/721E/748S, 469H/475E/749V, 469H/621S, 469H/621S/748S, 469H/748S, 469L, 469L/614A/695L, 469L/614A/695L/744S, 469L/744S, 469R, 469W, 469Y, 475E, 483A, 483C, 483G, 483L, 483Q, 483S, 483T, 485H/791S, 487G, 487L, 487R, 487S, 487V, 487Y, 489H, 506G, 506H, 506M, 506R, 506V, 507G, 507K, 507Q, 509F, 509M, 509V, 511A, 511D, 511L, 511N, 511R, 511V, 511Y, 514G, 514M, 514T, 514W, 517A, 517L, 517M, 517R, 517S, 517V, 517W, 520S, 520V, 532T, 532W, 532Y, 533K/542S, 538A, 538G, 538L, 538P, 538S, 539A, 539G, 539G/571A, 539L, 542A, 542G, 542K, 542P, 542R, 542S, 542W, 546G/699A, 547L, 547L/621S/721E/748S/749V, 549A, 549H, 549L, 549M, 549N, 549P, 549S, 549T, 549V, 549Y, 554S, 557P, 557P/621S, 580K, 584A, 584Q, 584V, 587G, 587M, 587S, 587T, 587V, 587W, 596W, 598A, 598C, 598F, 598F/611P, 598F/611P/614A, 598F/611P/708G, 598F/614A/700G/744G, 598F/708G, 598G, 598L, 598R, 598S, 598V, 602A, 602G, 602S, 602S/724H, 602V, 602W, 611C, 611I, 611K, 611L, 611P, 611P/614A, 611P/614A/708G/744S/789D, 611P/744S, 611R, 611S, 611T, 611V, 611W, 614A, 614A/700G/708G/744S/789D, 614A/744G, 614A/789D, 614G, 614P, 614T, 614W, 617C, 617D, 617G, 617H, 617L, 617R, 617S, 617V, 621H/745D, 621S, 630W, 652G, 652S, 652V, 689G, 689K, 689L, 689M, 689P, 689R, 689S, 689W, 691A, 691G, 691K, 691Q, 691R, 691S, 691T, 691W, 695A, 695E, 695L, 695L/708G/744G, 695L/744G, 695P, 695R, 695S, 695T, 699A, 700E, 700F, 700G, 700G/744G, 700G/767T, 700L, 700R, 700V, 700W, 700Y, 708A, 708G, 708R, 708T, 714G, 714L, 714P, 714R, 714S, 714V, 714Y, 721E, 721E/749V, 723A, 723Q, 723T, 723V, 725C, 725E, 725L, 725P, 725S, 725V, 725W, 741A, 741E, 741H, 741K, 741L, 741P, 744F, 744G, 744H, 744P, 744R, 744S, 744T, 744W, 745C, 745D, 745G, 745P, 745Q, 745V, 745W, 748D, 748R, 748S, 748S/749V, 748V, 767G, 767T, 777A, 777C, 777Q, 777T, 777V, 780A, 780R, 785Q, 785R, 785R/809V, 787E, 787G, 787K, 787P, 787W, 789A, 789D, 789E, 789G, 789P, 789Q, 789S, 789T, 789V, 789Y, 791A, 791G, 791H, 791L, 791R, 791T, 791V, 795D, 795S, 798T, 799M, 799Q, 811G, 811R, 811S, 811W, 817A, 817D, 817F, 817H, 817K, 817N, 817Q, 817S, 817V, 817W, 821D, 821G, 821K, 821L, 821Q, 821S, 836L, 838R, 838T, 838V, 839E, 839L, 839P, 839R, 839T, and 839V, or combinations thereof, wherein the amino acid positions are relative to the reference sequence of SEQ ID NO: 5094.

In some embodiments, the present invention provides an engineered polynucleotide encoding an engineered DNA polymerase comprising one or more substitutions or substitution sets selected from G9D/L691S, G9V/Q689G, R21S/Y695L/A700G, L45R, L45R/G268R, L45R/G268R/L302P/E423A, L45R/G268R/L302P/N467A/R621S/G748S/K749V, L45R/G268R/L302P/S469H, L45R/G268R/L302P/S469H/M547L/R557P/R721E/K749V, L45R/G268R/L302P/H475E/R489H, L45R/G268R/L302P/M547L/R557P/R621S/G748S/K749V, L45R/G268R/E423A/N467A/G748S, L45R/G268R/E423A/S469H/R721E, L45R/G268R/E423A/H475E/M547L/R721E/K749V, L45R/G268R/S469H, L45R/G268R/H475E, L45R/G268R/H475E/R489H, L45R/G268R/H475E/R621S, L45R/G268R/R489H/R621S/K749V, L45R/G268R/R621S/R721E, L45R/L302P, L45R/L302P/E423A/S469H/H475E, L45R/L302P/E423A/S469H/R621S, L45R/L302P/E423A/S469H/R621S/R721E/G748S/K749V, L45R/L302P/E423A/M547L/R621S/R721E, L45R/L302P/S469H/M547L/R721E, L45R/L302P/H475E/R621S/G748S/K749V, L45R/L302P/R621S, L45R/E423A, L45R/E423A/N467A, L45R/E423A/N467A/S469H/M547L/R721E/K749V, L45R/E423A/N467A/S469H/K749V, L45R/E423A/N467A/H475E/M547L/R621S/R721E/K749V, L45R/E423A/N467A/R621S, L45R/E423A/S469H/H475E, L45R/E423A/S469H/H475E/R621S/R721E/K749V, L45R/E423A/S469H/R489H/R621S, L45R/E423A/H475E/M547L/R721E, L45R/E423A/H475E/R557P/R621S/K749V, L45R/E423A/R489H, L45R/E423A/R489H/R557P, L45R/N467A/S469H/R557P, L45R/N467A/R621S, L45R/N467A/G748S, L45R/S469H/R721E, L45R/S469H/K749V, L45R/H475E/R489H/M547L/K749V, L45R/M547L/K749V, L45R/R557P, L45R/R721E, H83N/A700G/A744G, P95S/G268R/L302P/E423A/R489H/M547L/R557P/R721E/G748S/K749V, L116P, N228D/S708E, G268R, G268R/L302P/E423A/R557P/R721E, G268R/L302P/R721E, G268R/E423A, G268R/E423A/N467A/M547L, G268R/E423A/S469H, G268R/E423A/S469H/H475E, G268R/N467A, G268R/N467A/S469H/H475E, G268R/N467A/G748S, G268R/H475E/M547L, G268R/H475E/R557P/R721E/K749V, G268R/R489H/M547L, L296H/E433L, L302P, L302P/E423A/N467A/R621S/G748S, L302P/E423A/S469H/R489H/R721E/G748S/K749V, L302P/E423A/H475E/R489H/M547L/R621S, L302P/E423A/M547L/R721E, L302P/E423A/R557P, L302P/N467A/S469H/M547L/R557P, L302P/N467A/R489H/G748S/K749V, L302P/N467A/R621S, L302P/N467A/R721E/G748S, L302P/S469H, L302P/S469H/R489H/R621S/R721E/G748S, L302P/R489H, L302P/R489H/M547L/R621S/Q699A/K749V, L302P/R557P/R721E, L302P/R621S, L302P/R721E, L302P/K749V, A306E, A306L, A306M, A306N, A306P, A306R, A306S, A306T, A306T/K459E, A306V, A314S/V538L, S321A, S321I, S321W, R322A, R322C, R322D, R322G, R322L, R322M, R322P, R322S, R322T, E329K/G359T/E611P/A700G/A744S, A332C, A332D, A332I, A332L, A332M, A332Q, A332R, A332T, A332V, A335C, A335G, A335S, G339A, G339H, G339L, G339P, G339S, G339V, V341M/R532L, A344G, A344P, A344R, A344S, A344V, E345G, E345K, E345L, L351M, L351V, L354I/G359T/T408W/P598F, G359T, G359T/T408W/A412G, G359T/T408W/A412G/E611K/R614A/A744S, G359T/T408W/A412G/E611K/A744S/L789D, G359T/T408W/A412G/E611P/S708G/A744S, G359T/T408W/A412G/A700G/A744G, G359T/T408W/A412G/A744S, G359T/T408W/E433A/R614A/S708G, G359T/T408W/P598F/A700G, G359T/T408W/E611K/R614A/A744G, G359T/T408W/Y695L/A700G/A744G, G359T/T408W/Y695L/A744G, G359T/A412G, G359T/A412G/E433A/L436R/P598F/E611P/R614A, G359T/A412G/E433A/E611P/R614A/S708G, G359T/A412G/E433A/E611P/R614A/A744S/L789D, G359T/A412G/L436R/P598F/E611P/Y695L/A744G, G359T/A412G/S469L/E611P/Y695L/S708G/A744S, G359T/A412G/P598F, G359T/A412G/P598F/E611K/A744S, G359T/A412G/P598F/E611P/Y695L/A700G/S708G/A744S, G359T/A412G/Y695L/A700G/A744G, G359T/A412G/Y695L/S708G/A744G, G359T/E433A/E611P/R614A/A700G, G359T/E433A/E611P/Y695L/A700G/L789D, G359T/L436R/E611P/S708G/A744G, G359T/S469L/E611K, G359T/S469L/E611P/R614A, G359T/S469L/E611P/A700G, G359T/P598F/E611P/R614A/A744S, G359T/P598F/R614A/Y695L/S708G/A744S, G359T/P598F/A700G/A744G, G359T/P598F/A744S/L789D, G359T/E611K/R614A/A744G, G359T/E611P/R614A/Y695L/A700G/A744G, G359T/E611P/A700G/A744S, G359T/R614A, G359T/Y695L/A700G/L789D, G359T/A700G, G359T/A744S, G359T/L789D, D364G, D364P, D364R, D364T, D364V, G373F, G373K, G373P, G373S, G373V, P378C, P378F, P378G, P378L, P378R, P378Y, L385A, L385E, L385G, L385P, L385R, L385V, A386C, A386G, A386L, A386Q, A386S, A386V, A386W, Y387C/H630E, N393A, N393E, N393G, N393P, N393R, N393Y, E397A, E397D, E397G, E397L, E397N, E397P, E397R, E397V, V399E, V399G, V399L, V399P, V399Q, V399R, V399S, V399W, G404F, G404I, G404K, G404L, G404P, G404S, G404T, G404W, T408A, T408L, T408N, T408P, T408Q, T408R, T408W, T408W/A412G, T408W/A412G/E433A/L436R/E611P/R614A/A744G, T408W/A412G/E433A/P598F/E611P/A700G/S708G/A744S, T408W/A412G/L436R/P598F/S708G, T408W/A412G/L436R/E611P/R614A, T408W/A412G/S469L/H475Q/E611P/Y695L/S708G/A744S, T408W/A412G/P598F/E611P/R614A/S708G, T408W/A412G/P598F/A700G/A744S, T408W/A412G/E611P/Y695L/S708G, T408W/A412G/S708G/A744G, T408W/E433A/A700G/S708G/A744S, T408W/L436R/P598F/E611P/S708G/A744S, T408W/L436R/Y695L/S708G/A744G, T408W/P598F/E611P, T408W/P598F/E611P/R614A/A700G, T408W/P598F/Y695L/A700G, T408W/E611K/R614A/A744G, T408W/E611P/R614A/Y695L/S708G, T408W/E611P/R614A/S708G/A744G, T408W/E611P/A744S, T408W/A700G, T408W/A744G, T408W/A744S, E409G, A412F, A412G, A412G/E433A/P598F/E611P/A744G, A412G/E433A/E611P/A744S, A412G/L436R/E611K/R614A/A744S/L789D, A412G/L436R/A744G, A412G/S469L/E611P/Y695L/A744G, A412G/S469L/E611P/A700G/A744S, A412G/P598F/E611P/A744S, A412G/E611K/R614A, A412G/E611P/R614A/S708G/A744S/L789D, A412G/E611P/L789D, A412G/R614A/S708G, A412G/Y695L/L789D, A412G/S708G/A744G, A412K, A412L, A412P, A412Q, A412R, A412T/Q689V, K413E, K413G, K413H, K413L, K413R, K413S, K413T, K413W, L417A, L417G, L417Q, L417S, E423A, E423A/N467A/S469H, E423A/N467A/R489H/R621S/R721E, E423A/N467A/M547L/R557P/R621S/Q699A/R721E, E423A/N467A/R621S/R721E/K749V, E423A/N467A/R721E, E423A/S469H, E423A/S469H/H475E/R489H/R721E/G748S/K749V, E423A/S469H/R489H/R557P/R621S/R721E/K749V, E423A/S469H/M547L/R557P/R621S/K749V, E423A/S469H/M547L/R557P/K749V, E423A/S469H/R621S, E423A/S469H/R721E, E423A/R489H, E423A/R489H/R621S, E423A/R721E, E423A/G748S, E423G/P598F/A700G/S708G/A744S, N424E, N424G, N424L, N424T, L425C, L425S, L425T, L425Y, L426G, L426R, L426S, L426T, L426V, E433A, E433A/L436R/P598F/A700G/A744G, E433A/L436R/P598F/A744G, E433A/L436R/E611P/R614A, E433A/S469L/E611P, E433A/E611P/Y695L/A700G/A744G, E433A/A744S/L789D, E433G, E433Q, E433R, E433S, E433W, L436A, L436G, L436Q, L436R, L436R/E611K/R614A, L436R/E611P/R614A/Y695L, L436R/A700G/A744G, L436R/S708G/A744G, L436S, L436T, L436V, L436W, Y439A, Y439E, Y439L, Y439S, Y439W, E443C, E443T, L446S, R448E, R448L, R448W, V449G, V449W, N467A/S469H, N467A/R489H/M547L/R621S/G748S, N467A/M547L/R721E, N467A/R621S, N467A/R721E, N467A/R721E/G748S, N467A/G748S, N467A/K749V, N467C/S469H, N467H/S469H, N467M/S469H, N467R/S469H, N467T/S469H, N467V/S469H, N467W/S469H, S469E, S469H, S469H/E471A, S469H/E471F, S469H/E471H, S469H/E471L, S469H/E471M, S469H/E471Q, S469H/E471R, S469H/A473F, S469H/A473W, S469H/H475E/R621S/R721E/G748S, S469H/H475E/K749V, S469H/R621S, S469H/R621S/G748S, S469H/G748S, S469L, S469L/R614A/Y695L, S469L/R614A/Y695L/A744S, S469L/A744S, S469R, S469W, S469Y, H475E, V483A, V483C, V483G, V483L, V483Q, V483S, V483T, R485H/Q791S, A487G, A487L, A487R, A487S, A487V, A487Y, R489H, E506G, E506H, E506M, E506R, E506V, L507G, L507K, L507Q, L509F, L509M, L509V, P511A, P511D, P511L, P511N, P511R, P511V, P511Y, K514G, K514M, K514T, K514W, K517A, K517L, K517M, K517R, K517S, K517V, K517W, K520S, K520V, R532T, R532W, R532Y, E533K/L542S, V538A, V538G, V538L, V538P, V538S, D539A, D539G, D539G/T571A, D539L, L542A, L542G, L542K, L542P, L542R, L542S, L542W, E546G/Q699A, M547L, M547L/R621S/R721E/G748S/K749V, K549A, K549H, K549L, K549M, K549N, K549P, K549S, K549T, K549V, K549Y, Y554S, R557P, R557P/R621S, T580K, S584A, S584Q, S584V, D587G, D587M, D587S, D587T, D587V, D587W, R596W, P598A, P598C, P598F, P598F/E611P, P598F/E611P/R614A, P598F/E611P/S708G, P598F/R614A/A700G/A744G, P598F/S708G, P598G, P598L, P598R, P598S, P598V, K602A, K602G, K602S, K602S/R724H, K602V, K602W, E611C, E611I, E611K, E611L, E611P, E611P/R614A, E611P/R614A/S708G/A744S/L789D, E611P/A744S, E611R, E611S, E611T, E611V, E611W, R614A, R614A/A700G/S708G/A744S/L789D, R614A/A744G, R614A/L789D, R614G, R614P, R614T, R614W, A617C, A617D, A617G, A617H, A617L, A617R, A617S, A617V, R621H/R745D, R621S, R621S/R639H/G748S/K749V, R621S/G748S, R621S/K749V, H630A, H630C, H630F, H630G, H630M, H630S, H630T, H630W, A652G, A652S, A652V, Q689G, Q689K, Q689L, Q689M, Q689P, Q689R, Q689S, Q689W, L691A, L691G, L691K, L691Q, L691R, L691S, L691T, L691W, Y695A, Y695E, Y695L, Y695L/S708G/A744G, Y695L/A744G, Y695P, Y695R, Y695S, Y695T, Q699A, A700E, A700F, A700G, A700G/A744G, A700G/A767T, A700L, A700R, A700V, A700W, A700Y, S708A, S708G, S708R, S708T, A714G, A714L, A714P, A714R, A714S, A714V, A714Y, R721E, R721E/K749V, G723A, G723Q, G723T, G723V, T725C, T725E, T725L, T725P, T725S, T725V, T725W, D741A, D741E, D741H, D741K, D741L, D741P, A744F, A744G, A744H, A744P, A744R, A744S, A744T, A744W, R745C, R745D, R745G, R745P, R745Q, R745V, R745W, G748D, G748R, G748S, G748S/K749V, G748V, A767G, A767T, L777A, L777C, L777Q, L777T, L777V, Q780A, Q780R, G785Q, G785R, G785R/A809V, R787E, R787G, R787K, R787P, R787W, L789A, L789D, L789E, L789G, L789P, L789Q, L789S, L789T, L789V, L789Y, Q791A, Q791G, Q791H, Q791L, Q791R, Q791T, Q791V, E795D, E795S, L798T, E799M, E799Q, L811G, L811R, L811S, L811W, E817A, E817D, E817F, E817H, E817K, E817N, E817Q, E817S, E817V, E817W, P821D, P821G, P821K, P821L, P821Q, P821S, W836L, A838R, A838T, A838V, A839E, A839L, A839P, A839R, A839T, and A839V, or combinations thereof, wherein the amino acid positions are relative to the reference sequence of SEQ ID NO: 5094.

In some embodiments, the present invention provides an engineered polynucleotide encoding an engineered DNA polymerase comprising one or more substitutions or substitution sets selected from 45/268/302/469, 45/268/475, 45/268/475/489, 45/268/621/721, 45/423/467/469/749, 45/721, 268, 268/475/547, 302, 302/489/547/621/699/749, 302/721, 302/749, 306, 359, 359/412/433/611/614/744/789, 359/436/611/708/744, 385, 408, 412, 412/598/611/744, 417, 423/467/721, 426, 433, 436, 436/708/744, 467/469, 469, 469/475/621/721/748, 469/475/749, 469/621/748, 469/748, 489, 511, 517, 547, 549, 557, 598, 598/611/708, 611, 614, 621/639/748/749, 630, 695, 699, 700, 708, 714, 721, 725, 741, 744, 748, 787, 789, 791, 795, and 817, or combinations thereof, wherein the amino acid positions are relative to the reference sequence of SEQ ID NO: 5094.

In some embodiments, the present invention provides an engineered polynucleotide encoding an engineered DNA polymerase comprising one or more substitutions or substitution sets selected from 45R/268R/302P/469H, 45R/268R/475E, 45R/268R/475E/489H, 45R/268R/621S/721E, 45R/423A/467A/469H/749V, 45R/721E, 268R, 268R/475E/547L, 302P, 302P/489H/547L/621S/699A/749V, 302P/721E, 302P/749V, 306P, 359T, 359T/412G/433A/611P/614A/744S/789D, 359T/436R/611P/708G/744G, 385V, 408W, 412G, 412G/598F/611P/744S, 412P, 417G, 423A/467A/721E, 426R, 433A, 436R, 436R/708G/744G, 467A/469H, 469H/475E/621S/721E/748S, 469H/475E/749V, 469H/621S/748S, 469H/748S, 469L, 489H, 511R, 517R, 547L, 549L, 557P, 598F, 598F/611P/708G, 611K, 611P, 614A, 621S/639H/748S/749V, 630M, 695L, 695P, 699A, 700G, 700R, 708G, 714S, 721E, 725P, 741H, 741L, 744G, 744S, 748S, 787W, 789D, 791R, 795S, 817A, and 817K, or combinations thereof, wherein the amino acid positions are relative to the reference sequence of SEQ ID NO: 5094.

In some embodiments, the present invention provides an engineered polynucleotide encoding an engineered DNA polymerase comprising one or more substitutions or substitution sets selected from L45R/G268R/L302P/S469H, L45R/G268R/H475E, L45R/G268R/H475E/R489H, L45R/G268R/R621S/R721E, L45R/E423A/N467A/S469H/K749V, L45R/R721E, G268R, G268R/H475E/M547L, L302P, L302P/R489H/M547L/R621S/Q699A/K749V, L302P/R721E, L302P/K749V, A306P, G359T, G359T/A412G/E433A/E611P/R614A/A744S/L789D, G359T/L436R/E611P/S708G/A744G, L385V, T408W, A412G, A412G/P598F/E611P/A744S, A412P, L417G, E423A/N467A/R721E, L426R, E433A, L436R, L436R/S708G/A744G, N467A/S469H, S469H/H475E/R621S/R721E/G748S, S469H/H475E/K749V, S469H/R621S/G748S, S469H/G748S, S469L, R489H, P511R, K517R, M547L, K549L, R557P, P598F, P598F/E611P/S708G, E611K, E611P, R614A, R621S/R639H/G748S/K749V, H630M, Y695L, Y695P, Q699A, A700G, A700R, S708G, A714S, R721E, T725P, D741H, D741L, A744G, A744S, G748S, R787W, L789D, Q791R, E795S, E817A, and E817K, or combinations thereof, wherein the amino acid positions are relative to the reference sequence of SEQ ID NO: 5094.

The present invention also provides engineered polynucleotides encoding engineered DNA polymerases or functional fragments thereof, wherein the DNA polymerases comprise polypeptide sequences having at least 75% or more sequence identity to a reference sequence comprising residues 12 to 841 of SEQ ID NO: 5136, or to a reference sequence of SEQ ID NO: 5136, wherein the polypeptide sequence comprises one or more substitutions relative to the reference sequence comprising residues 12 to 841 of SEQ ID NO: 5136, or to the reference sequence of SEQ ID NO: 5136.

In some embodiments, the present invention provides an engineered polynucleotide encoding an engineered DNA polymerase comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5136 or to the reference sequence of SEQ ID NO: 5136.

In some embodiments, the present invention provides an engineered polynucleotide encoding an engineered DNA polymerase comprising one or more substitutions or substitution sets selected from 359/408/614, 359/412/436/598/611/614/695/700/708/744, 359/412/598/611/614/700/708/744, 359/412/611/708/744, 359/433/744, 359/436/598, 359/469/700/744, 359/598/611, 359/598/611/695/708, 359/598/695, 359/611/700/708, 359/744, 359/789, 408, 408/412/436/614/700/744, 408/412/469, 408/412/611, 408/412/744, 408/433/436/611/614/744, 408/433/436/695/708/744, 408/611, 412, 412/433, 412/611/614/708/744/789, 412/700/744, 433/436/598/611/614/708/744, 433/436/611/614/744, 433/436/611/744, 433/436/744, 433/614/700/744, 433/708, 436/598, 436/598/614/695/744, 436/611, 436/611/614, 436/611/614/695/700/744, 436/611/708, 436/744, 598/611/614/708/744, 598/614/695/700/708/744, 611, 611/614, 611/614/695/700/708/744, 611/708/744, 614/744, 614/789, 695, 695/700/744, 700, 700/744, and 744/789, or combinations thereof, wherein the amino acid positions are relative to the reference sequence of SEQ ID NO: 5136.

In some embodiments, the present invention provides an engineered polynucleotide encoding an engineered DNA polymerase comprising one or more substitutions or substitution sets selected from 359T/408W/614R, 359T/412G/436R/598F/611K/614R/695L/700G/708G/744S, 359T/412G/598F/611P/614R/700G/708G/744S, 359T/412G/611K/708G/744G, 359T/433A/744G, 359T/436R/598F, 359T/469L/700G/744G, 359T/598F/611K, 359T/598F/611K/695L/708G, 359T/598F/695L, 359T/611K/700G/708G, 359T/744S, 359T/789D, 408W, 408W/412G/436R/614R/700G/744S, 408W/412G/469L, 408W/412G/611P, 408W/412G/744G, 408W/433A/436R/611P/614R/744G, 408W/433A/436R/695L/708G/744S, 408W/611P, 412G, 412G/433A, 412G/611K/614R/708G/744S/789D, 412G/700G/744S, 433A/436R/598F/611P/614R/708G/744S, 433A/436R/611P/614R/744S, 433A/436R/611P/744G, 433A/436R/744G, 433A/614R/700G/744S, 433A/708G, 436R/598F, 436R/598F/614R/695L/744G, 436R/611P, 436R/611P/614R, 436R/611P/614R/695L/700G/744S, 436R/611P/708G, 436R/744S, 598F/611P/614R/708G/744G, 598F/614R/695L/700G/708G/744S, 611P, 611P/614R, 611P/614R/695L/700G/708G/744S, 611P/708G/744S, 614R/744G, 614R/789D, 695L, 695L/700G/744G, 700G, 700G/744S, and 744S/789D, or combinations thereof, wherein the amino acid positions are relative to the reference sequence of SEQ ID NO: 5136.

In some embodiments, the present invention provides an engineered polynucleotide encoding an engineered DNA polymerase comprising one or more substitutions or substitution sets selected from G359T/T408W/A614R, G359T/A412G/L436R/P598F/E611K/A614R/Y695L/A700G/S708G/A744S, G359T/A412G/P598F/E611P/A614R/A700G/S708G/A744S, G359T/A412G/E611K/S708G/A744G, G359T/E433A/A744G, G359T/L436R/P598F, G359T/S469L/A700G/A744G, G359T/P598F/E611K, G359T/P598F/E611K/Y695L/S708G, G359T/P598F/Y695L, G359T/E611K/A700G/S708G, G359T/A744S, G359T/L789D, T408W, T408W/A412G/L436R/A614R/A700G/A744S, T408W/A412G/S469L, T408W/A412G/E611P, T408W/A412G/A744G, T408W/E433A/L436R/E611P/A614R/A744G, T408W/E433A/L436R/Y695L/S708G/A744S, T408W/E611P, A412G, A412G/E433A, A412G/E611K/A614R/S708G/A744S/L789D, A412G/A700G/A744S, E433A/L436R/P598F/E611P/A614R/S708G/A744S, E433A/L436R/E611P/A614R/A744S, E433A/L436R/E611P/A744G, E433A/L436R/A744G, E433A/A614R/A700G/A744S, E433A/S708G, L436R/P598F, L436R/P598F/A614R/Y695L/A744G, L436R/E611P, L436R/E611P/A614R, L436R/E611P/A614R/Y695L/A700G/A744S, L436R/E611P/S708G, L436R/A744S, P598F/E611P/A614R/S708G/A744G, P598F/A614R/Y695L/A700G/S708G/A744S, E611P, E611P/A614R, E611P/A614R/Y695L/A700G/S708G/A744S, E611P/S708G/A744S, A614R/A744G, A614R/L789D, Y695L, Y695L/A700G/A744G, A700G, A700G/A744S, and A744S/L789D, or combinations thereof, wherein the amino acid positions are relative to the reference sequence of SEQ ID NO: 5136.

In some embodiments, the present invention provides an engineered polynucleotide encoding an engineered DNA polymerase comprising one or more substitutions or substitution sets selected from 359/598/695, 408/412/436/614/700/744, 408/611, 412, 433/614/700/744, 436/598, 436/611, 436/611/614/695/700/744, and 700, or combinations thereof, wherein the amino acid positions are relative to the reference sequence of SEQ ID NO: 5136.

In some embodiments, the present invention provides an engineered polynucleotide encoding an engineered DNA polymerase comprising one or more substitutions or substitution sets selected from 359T/598F/695L, 408W/412G/436R/614R/700G/744S, 408W/611P, 412G, 433A/614R/700G/744S, 436R/598F, 436R/611P, 436R/611P/614R/695L/700G/744S, and 700G, or combinations thereof, wherein the amino acid positions are relative to the reference sequence of SEQ ID NO: 5136.

In some embodiments, the present invention provides an engineered polynucleotide encoding an engineered DNA polymerase comprising one or more substitutions or substitution sets selected from G359T/P598F/Y695L, T408W/A412G/L436R/A614R/A700G/A744S, T408W/E611P, A412G, E433A/A614R/A700G/A744S, L436R/P598F, L436R/E611P, L436R/E611P/A614R/Y695L/A700G/A744S, and A700G, or combinations thereof, wherein the amino acid positions are relative to the reference sequence of SEQ ID NO: 5136.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2, 178, 994, 1858, 2176, 2310, 2994, 3796, 4182, 5094 and/or 5136, wherein the polypeptide sequence optionally comprises one or more amino acid substitutions relative to SEQ ID NO: 2, 178, 994, 1858, 2176, 2310, 2994, 3796, 4182, 5094 and/or 5136, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 178, wherein the polypeptide sequence optionally comprises one or more amino acid substitutions relative to SEQ ID NO: 178, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 994, wherein the polypeptide sequence optionally comprises one or more amino acid substitutions relative to SEQ ID NO: 994, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 1858, wherein the polypeptide sequence optionally comprises one or more amino acid substitutions relative to SEQ ID NO: 1858, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2176, wherein the polypeptide sequence optionally comprises one or more amino acid substitutions relative to SEQ ID NO: 2176, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2310, wherein the polypeptide sequence optionally comprises one or more amino acid substitutions relative to SEQ ID NO: 2310, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2994, wherein the polypeptide sequence optionally comprises one or more amino acid substitutions relative to SEQ ID NO: 2994, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 3796, wherein the polypeptide sequence optionally comprises one or more amino acid substitutions relative to SEQ ID NO: 3796, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 4182, wherein the polypeptide sequence optionally comprises one or more amino acid substitutions relative to SEQ ID NO: 4182, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5094, wherein the polypeptide sequence optionally comprises one or more amino acid substitutions relative to SEQ ID NO: 5094, with the proviso that the polypeptide sequence does not comprise the sequence comprising residues 12 to 841 of SEQ ID NO:2.

In some embodiments, the polynucleotide encodes an engineered DNA polymerase comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5136, wherein the polypeptide sequence optionally comprises one or more amino acid substitutions relative to SEQ ID NO: 5136, with the proviso that the polypeptide sequence does not comprise the sequence comprising residues 12 to 841 of SEQ ID NO:2.

In some embodiments, for each of the foregoing embodiments, the specific amino acid substitutions described herein for the substitution or substitution set can be used for the encoded engineered DNA polymerase polypeptide.

In some embodiments, the polynucleotide encodes an engineered DNA polymerase comprising a polypeptide sequence comprising at least one substitution provided in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1, wherein the substitution is relative to SEQ ID NO: 2, 178, 994, 1858, 2176, 2310, 2994, 3796, 4182, 5094, and/or 5136.

In some embodiments, the polynucleotide encodes an engineered DNA polymerase comprising at least one substitution or substitution set provided in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1, wherein the substitution or substitution set is relative to SEQ ID NO: 2, 178, 994, 1858, 2176, 2310, 2994, 3796, 4182, 5094, and/or 5136.

In some embodiments, the polynucleotide encodes an engineered DNA polymerase comprising a polypeptide sequence comprising at least 75%, 80%, 85%, 86%, 887%, 88%, 89%, 90%, 91%, 92%, 93%, 94%, 95%, 96%, 97%, 98%, 99% or more sequence identity to a sequence comprising residues 12 to 841 of an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1. In some embodiments, the polynucleotide encodes an engineered DNA polymerase comprising a polypeptide sequence comprising at least 75%, 80%, 85%, 86%, 887%, 88%, 89%, 90%, 91%, 92%, 93%, 94%, 95%, 96%, 97%, 98%, 99% or more sequence identity to a sequence of an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1. In some embodiments, the encoded engineered DNA polymerase comprises a polypeptide sequence comprising residues 12 to 841 of an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1. In some embodiments, the encoded engineered DNA polymerase comprises a polypeptide sequence corresponding to an even numbered SEQ ID NO. set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1.

In some embodiments, the polynucleotide encodes an engineered DNA polypeptide sequence comprising residues 12 to 841 of an even-numbered sequence of SEQ ID NO: 4-3258 or 3268-6646, or a fragment thereof, wherein the polypeptide sequence has optionally 1, 2, 3, 4, 5, 6, 7, 8, 9, or up to 10 substitutions in the polypeptide sequence, as compared with SEQ ID NO: 2.

In some embodiments, the polynucleotide encodes an engineered DNA polypeptide comprising a sequence of an even-numbered sequence of SEQ ID NO: 4-3258 or 3268-6646, or a fragment thereof, wherein the polypeptide sequence has optionally 1, 2, 3, 4, 5, 6, 7, 8, 9, or up to 10 substitutions in the polypeptide sequence, as compared with SEQ ID NO: 2.

In some embodiments, the encoded engineered DNA polymerase polypeptide includes 1, 2, 3, 4, up to 5 substitutions in the polypeptide sequence. In some embodiments, the engineered DNA polymerase polypeptide includes 1, 2, 3, or 4 substitutions in the polypeptide sequence. In some embodiments, the substitutions comprise non-conservative of conservative substitutions. In some embodiments, the substitutions comprise conservative substitutions. In some embodiments, the substitutions comprise non-conservative substitutions. In some embodiments, guidance on non-conservative and conservative substitutions are provided by the variants disclosed herein.

In some embodiments, the polynucleotide encodes an engineered DNA polymerase polypeptide comprising a polypeptide sequence comprising residues 12 to 841 of SEQ ID NO: 178, 994, 1858, 2176, 2310, 2994, 3796, 4182, 5094, and/or 5136, or encodes a polypeptide sequence of SEQ ID NO: 178, 994, 1858, 2176, 2310, 2994, 3796, 4182, 5094, and/or 5136, wherein the polypeptide sequence has optionally 1, 2, 3, 4, 5, 6, 7, 8, 9, or up to 10 substitutions in the polypeptide sequence. In some embodiments, the encoded DNA polymerase includes 1, 2, 3, 4, up to 5 substitutions in the polypeptide sequence. In some embodiments, the encoded DNA polymerase includes 1, 2, 3, or 4 substitutions in the polypeptide sequence.

In some embodiments, the polynucleotide comprises a polynucleotide sequence comprising at least 75%, 80%, 85%, 90%, 91%, 92%, 93%, 94%, 95%, 96%, 97%, 98%, 99%, or more sequence identity to a reference polynucleotide sequence comprising nucleotide residues 34 to 2529 of SEQ ID NO: 1, or to a reference polynucleotide sequence of SEQ ID NO: 1, encoding an engineered DNA polymerase or a functional fragment thereof, wherein the engineered polypeptide comprises one or more substitutions at one or more amino acid positions relative to the reference sequence comprising residues 12 to 841 of SEQ ID NO:2, or to the reference sequence of SEQ ID NO: 2.

In some embodiments, the polynucleotide encoding an engineered DNA polymerase comprises a polynucleotide sequence comprising at least 75%, 80%, 85%, 86%, 87%, 88%, 89%, 90%, 91%, 92%, 93%, 94%, 95%, 96%, 97%, 98%, 99% or more sequence identity to a polynucleotide sequence of nucleotide residues 34 to 2529 of an odd-numbered sequence of SEQ ID NO: 3-3257 or 3267-6645, wherein the polynucleotide encodes an engineered DNA polymerase, as described herein.

In some embodiments, the polynucleotide encoding an engineered DNA polymerase comprises a polynucleotide sequence of nucleotide residues 34 to 2529 of an odd-numbered sequence of SEQ ID NO: 3-3257 or 3267-6645.

In some embodiments, the polynucleotide encoding an engineered DNA polymerase comprises a polynucleotide sequence comprising at least 75%, 80%, 85%, 86%, 87%, 88%, 89%, 90%, 91%, 92%, 93%, 94%, 95%, 96%, 97%, 98%, 99% or more sequence identity to an odd-numbered sequence of SEQ ID NO: 3-3257 or 3267-6645, wherein the polynucleotide encodes an engineered DNA polymerase, as described herein.

In some embodiments, the polynucleotide encoding an engineered DNA polymerase comprises a polynucleotide sequence comprising at least about 75%, about 80%, about 85%, about 86%, about 87%, about 88%, about 89%, about 90%, about 91%, about 92%, about 93%, about 94%, about 95%, about 96%, about 97%, about 98%, about 99% or more sequence identity to an odd-numbered sequence of SEQ ID NO: 3-3257 or 3267-6645, wherein the polynucleotide encodes an engineered DNA polymerase, as described herein.

In some embodiments, the polynucleotide encoding an engineered DNA polymerase comprises the sequence an odd-numbered sequence of SEQ ID NO: 3-3257 or 3267-6645.

In some embodiments, an isolated polynucleotide encoding any of the engineered DNA polymerase polypeptides herein is manipulated in a variety of ways to facilitate expression of the DNA polymerase polypeptide. In some embodiments, the polynucleotides encoding the DNA polymerase polypeptides comprise expression vectors where one or more control sequences is present to regulate the expression of the DNA polymerase polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators. In some embodiments, suitable promoters are selected based on the host cells selection. For bacterial host cells, suitable promoters for directing transcription of the nucleic acid constructs of the present invention, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA, 1978, 75:3727-3731), as well as the tac promoter (See e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GALI), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast, 1992, 8:423-488).

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the DNA polymerase polypeptide. Any suitable terminator which is functional in the host cell of choice finds use in the present invention. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the DNA polymerase polypeptide. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

In some embodiments, the control sequence is also a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (See e.g., Guo and Sherman, Mol. Cell. Biol., 1995, 15:5983-5990).

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase.

In some embodiments, the control sequence is also a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GALI system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present invention provides a recombinant expression vector comprising a polynucleotide encoding an engineered DNA polymerase polypeptide, and one or more expression regulating regions such as a promoter and a terminator, a replication origin, etc., depending on the type of hosts into which they are to be introduced. In some embodiments, the various nucleic acid and control sequences described herein are joined together (i.e., operably linked) to produce recombinant expression vectors which include one or more convenient restriction sites to allow for insertion or substitution of the nucleic acid sequence encoding the DNA polymerase polypeptide at such sites. Alternatively, in some embodiments, the nucleic acid sequence of the present invention is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the DNA polymerase polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof. In another aspect, the present invention provides a host cell comprising at least one polynucleotide encoding at least one engineered DNA polymerase polypeptide of the present invention, the polynucleotide(s) being operatively linked to one or more control sequences for expression of the engineered DNA polymerase enzyme(s) in the host cell. Host cells suitable for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells also include various Escherichia coli strains (e.g., W3110 (ΔfhuA) and BL21).

Accordingly, in another aspect, the present invention provides methods of producing the engineered DNA polymerase polypeptides, where the methods comprise culturing a host cell capable of expressing a polynucleotide encoding the engineered DNA polymerase polypeptide under conditions suitable for expression of the polypeptide. In some embodiments, the methods further comprise the step(s) of isolating and/or purifying the DNA polymerase polypeptides, as described herein.

Appropriate culture media and growth conditions for host cells are well known in the art. It is contemplated that any suitable method for introducing polynucleotides for expression of the DNA polymerase polypeptides into cells will find use in the present invention. Suitable techniques include, but are not limited to electroporation, biolistic particle bombardment, liposome mediated transfection, calcium chloride transfection, and protoplast fusion.

Engineered DNA polymerase polypeptides with the properties disclosed herein can be obtained by subjecting the polynucleotide encoding the naturally occurring or engineered DNA polymerase polypeptide to any suitable mutagenesis and/or directed evolution methods known in the art, and/or as described herein. An exemplary directed evolution technique is mutagenesis and/or DNA shuffling (See e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 1998, 16:258-261), mutagenic PCR (See e.g., Caldwell et al., PCR Methods Appl., 1994, 3: S136-S140), and cassette mutagenesis (See e.g., Black et al., Proc. Natl. Acad. Sci. USA, 1996, 93:3525-3529).

Mutagenesis and directed evolution methods can be readily applied to DNA polymerase-encoding polynucleotides to generate variant libraries that can be expressed, screened, and assayed. Any suitable mutagenesis and directed evolution methods find use in the present invention and are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related PCT and non-US counterparts; Ling et al., Anal. Biochem., 1997, 254 (2): 157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 370:389-391; Stemmer, Proc. Nat. Acad. Sci. USA, 1994, 91:10747-10751; EP 3 049 973; WO 95/22625; WO 97/0078; WO 97/35966; WO 98/27230; WO 00/42651; WO 01/75767; WO 2009/152336; and WO 2015/048573, all of which are incorporated herein by reference).

In some embodiments, the enzyme clones obtained following mutagenesis treatment are screened by subjecting the enzyme preparations to a defined temperature (or other assay conditions) and measuring the amount of enzyme activity remaining after heat treatments or other suitable assay conditions. Clones containing a polynucleotide encoding a DNA polymerase polypeptide are then isolated from the gene, sequenced to identify the nucleotide sequence changes (if any), and used to express the enzyme in a host cell. Measuring enzyme activity from the expression libraries can be performed using any suitable method known in the art (e.g., standard biochemistry techniques, such as HPLC analysis).

For engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tet. Lett., 1981, 22:1859-69; and Matthes et al., EMBO J., 1984, 3:801-05), as it is typically practiced in automated synthetic methods. According to the phosphoramidite method, oligonucleotides are synthesized (e.g., in an automatic DNA synthesizer, purified, annealed, ligated and cloned in appropriate vectors).

Accordingly, in some embodiments, a method for preparing the engineered DNA polymerase polypeptide can comprise: (a) synthesizing a polynucleotide encoding a polypeptide comprising an amino acid sequence selected from the amino acid sequence of any variant as described herein, and (b) expressing the DNA polymerase polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

The expressed engineered DNA polymerase polypeptide can be evaluated for any desired improved property or a combination of properties (e.g., activity, selectivity, fidelity, stability, thermostability, tolerance to various pH levels, protease sensitivity, etc.) using any suitable assay known in the art, including but not limited to the assays and conditions described herein.

In some embodiments, any of the engineered DNA polymerase polypeptides expressed in a host cell are recovered from the cells and/or the culture medium using any one or more of the well-known techniques for protein purification, including, among others, lysozyme treatment, sonication, filtration, salting-out, ultra-centrifugation, and chromatography.

Chromatographic techniques for isolation of the DNA polymerase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying a particular enzyme depends, in part, on factors such as net charge, hydrophobicity, hydrophilicity, molecular weight, molecular shape, etc., and will be apparent to those having skill in the art. In some embodiments, affinity techniques may be used to isolate the improved DNA polymerase enzymes. For affinity chromatography purification, any antibody that specifically binds a DNA polymerase polypeptide of interest may find use. For the production of antibodies, various host animals, including but not limited to rabbits, mice, rats, etc., are immunized by injection with a DNA polymerase polypeptide, or a fragment thereof. In some embodiments, the DNA polymerase polypeptide or fragment is attached to a suitable carrier, such as BSA, by means of a side chain functional group or linkers attached to a side chain functional group.

In some embodiments, the engineered DNA polymerase polypeptide is produced in a host cell by a method comprising culturing a host cell (e.g., an E. coli strain) comprising a polynucleotide sequence encoding an engineered DNA polymerase polypeptide as described herein under conditions conducive to the production of the engineered DNA polymerase polypeptide and recovering the engineered DNA polymerase polypeptide from the cells and/or culture medium. In some embodiments, the host cell produces more than one engineered DNA polymerase polypeptide.

In some embodiments, the present invention provides a method of producing an engineered DNA polymerase polypeptide comprising culturing a recombinant cell comprising a polynucleotide sequence encoding an engineered DNA polymerase polypeptide as described herein, under suitable culture conditions to allow the production of the engineered DNA polymerase polypeptide and optionally recovering the engineered DNA polymerase polypeptide from the culture and/or cultured cells. In some embodiments, the host cell produces more than one engineered DNA polymerase polypeptide. In some embodiments, the host cell is a bacterial cell.

In some embodiments, the isolated or purified engineered DNA polymerase polypeptides are combined with other ingredients and compounds to provide compositions and formulations comprising the engineered DNA polymerase polypeptide as appropriate for different applications and uses (e.g., diagnostic methods and compositions). In some embodiments, a composition comprises at least one engineered DNA polymerase of the present invention. In some embodiments, the composition further comprises a buffer. In some embodiments, the composition further comprises a substrate, such as nucleotide substrates (e.g., dNTPs, dNTP analogs, and/or modified dNTPs) and/or at least one primer, e.g., complementary to a target nucleic acid. In some embodiments, the composition can further comprise a DNA polymerase (e.g., a second DNA polymerase) other than the engineered DNA polymerase. In some embodiments, the second DNA polymerase is a second thermostable DNA polymerase, for example Taq or Pfu polymerase, or a reverse transcriptase, such as those useful in RT-PCR coupled reactions. In some embodiments, the composition includes a probe or indicator, such as a nucleic acid binding dye (e.g., SYBR® Green), for detecting and/or quantitating the amount of product formed (e.g., in a qRT-PCR reaction).

### Uses of Engineered DNA Polymerase Polypeptides and Kits

In another aspect, the present invention provides uses of the engineered DNA polymerases for diagnostic and molecular biological uses, such as for detecting the presence of a target nucleic acid, nucleic acid sequencing, and direct/indirect sequence of nucleic acids. In addition, the present invention provides a novel buffer suitable for use with the engineered DNA polymerases and kits provided herein.

In some embodiments, the engineered DNA polymerase is used in preparing a complementary DNA of a target DNA. In some embodiments, a method of preparing a complementary DNA of a target DNA comprises contact a target DNA with an engineered DNA polymerase described herein in presence of substrates sufficient for producing a complementary DNA under reaction conditions suitable for production of a complementary DNA to all or a portion (i.e., whole or in part) of the target DNA. As discussed herein and known in the art, substrates include nucleotides (e.g., dNTPs) for DNA polymerase activity and/or oligonucleotide primers. Primers can be to a specific sequence of the target nucleic acid, or random primers, such as for generation of DNA libraries.

In some embodiments, the target DNA is any DNA appropriate as a template for the engineered DNA polymerase, including, but not limited to, genomic DNA, mitochondrial DNA, cell-free DNA (e.g., obtained from blood/serum), bacterial DNA, fungal DNA, or viral DNA.

In some embodiments, the engineered DNA polymerase is useful in diagnostic applications, e.g., for detecting the presence of a target nucleic acid, including RNA and DNA. In some embodiments, a method for detecting presence of a target DNA comprises reacting a sample suspected of containing a target DNA with an engineered DNA polymerase described herein in presence of substrates under conditions suitable for DNA polymerase mediated production of a DNA complementary to all or a portion (i.e., whole or in part) of the target DNA, and detecting the presence of the complementary DNA. In some embodiments, a target RNA can be detected by using a reverse transcriptase to produce a corresponding target DNA complementary to the target RNA.

In some embodiments, the sample can be any material or substance suspected of containing a target nucleic acid. In some embodiments, the sample is a biological sample, such as biopsy and autopsy samples, frozen sections taken for histological purposes, blood, plasma, serum, sputum, stool, tears, mucus, hair, skin, etc. In some embodiments, the biological sample are cells or viruses, such as from a bacterial culture, virus culture, or cell culture.

In some embodiments for detecting a target DNA, the detection of the complementary DNA product can be effectuated by known methods in the art. In some embodiments, the complementary DNA is detected by amplifying the complementary DNA, such as by polymerase chain reaction (PCR) or isothermal amplification. In some embodiments, a suitable isothermal amplification is by loop mediated isothermal amplification (LAMP). In some embodiments for detecting the presence of a target RNA, the reaction with a reverse transcriptase is conducted separately from the amplification reaction with the DNA polymerase. In some embodiments, where the amplification is by PCR, the reverse transcriptase reaction and the PCR is a one-step RT-PCR (i.e., performed in a single reaction simultaneously). In some embodiments, where the amplification is by PCR, the reverse transcriptase reaction and the PCR is a two-step RT-PCR (i.e., performed separately).

In some embodiments, the engineered DNA polymerase is used for sequencing nucleic acids. Various methods for sequencing DNA, particularly NGS sequencing methods, are well known in the art.

In a further aspect, the present invention provides a kit comprising at least one engineered DNA polymerase disclosed herein. In some embodiments, the kit further comprises a buffer, a nucleotide substrate, and/or an oligonucleotide primer. In some embodiments, the kit can include multiple (e.g., two or more) oligonucleotide primers, for example to different portions of a target nucleic acid. In some embodiments, the kit comprises a second DNA polymerase, such as Taq or Pfu DNA polymerase or reverse transcriptase (e.g., for coupled RT-PCR reaction).

### EXPERIMENTAL

The following Examples, including experiments and results achieved, are provided for illustrative purposes only and are not to be construed as limiting the present invention.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and ug (micrograms); L and l (liter); ml and mL (milliliter); cm (centimeters); mm (millimeters); um and μm (micrometers); sec. (seconds); min(s) (minute(s)); h(s) and hr(s) (hour(s)); Ω (ohm); μf (microfarad); U (units); MW (molecular weight); rpm (rotations per minute); rcf (relative centrifugal force); psi and PSI (pounds per square inch); ° C. (degrees Centigrade); RT and rt (room temperature); NGS (next-generation sequencing); ds (double stranded); ss (single stranded); CDS (coding sequence); DNA (deoxyribonucleic acid); RNA (ribonucleic acid); dNTPs (deoxynucleoside triphosphates); gDNA (human genomic DNA); E. coli W3110 (commonly used laboratory E. coli strain, available from the Coli Genetic Stock Center [CGSC], New Haven, CT); HTP (high throughput); HPLC (high pressure liquid chromatography); MCYP (microcyp); ddH2O (double distilled water); PBS (phosphate buffered saline); SDS (sodium dodecyl sulfate); BSA (bovine serum albumin); DTT (dithiothreitol); CAM (chloramphenicol); CAT (chloramphenicol acetyltransferase); LB (Luria-Bertani broth); TB (Terrific Broth); GITC (guanidinium isothiocyanate); IPTG (isopropyl β-D-1-thiogalactopyranoside); FIOPC (fold improvements over positive control); SPRI (solid phase reversible immobilization); Sigma-Aldrich (Sigma-Aldrich, St. Louis, MO); Biotium (Biotium, Inc., Fremont, CA); Eppendorf (Eppendorf North America, Enfield, CT); IDT (Integrated DNA Technologies, Coralville, IA); Twist (Twist Bioscience, South San Francisco, CA); Codexis (Codexis, Inc., Redwood City, CA); Promega (Promega, Inc., Madison, WI); Perkin Elmer (Perkin Elmer, Inc, Waltham, MA); Harvard Apparatus (Harvard Apparatus, Holliston, MA); Millipore (Millipore, Corp., Billerica MA); Covaris (Covaris, Inc., Woburn, MA); MagBio (MagBio Genomics, Inc., Gaithersburg, MD); Qiagen (Qiagen Inc., Germantown, MD); Illumina (Illumina, Inc., San Diego, CA); BD Biosciences (BD Biosciences, San Jose, CA); Difco (Difco Laboratories, BD Diagnostic Systems, Detroit, MI); Kuhner (Adolf Kuhner, AG, Basel, Switzerland); Zymo (Zymo Research, Irvine, CA); Agilent (Agilent Technologies, Inc., Santa Clara, CA); ThermoFisher (part of Thermo Fisher Scientific, Waltham, MA); GE Healthcare (GE Healthcare Bio-Sciences, Piscataway, NJ); and Bio-Rad (Bio-Rad Laboratories, Hercules, CA).

### Example 1

E. coli Expression Hosts Containing Recombinant DNA Polymerase Genes

The initial DNA polymerase enzyme used to produce the variants of the present invention was SEQ ID NO: 2, cloned into the expression vector pCK110900 (See e.g., U.S. Pat. No. 7,629,157 and US Pat. Appln. Publn. 2016/0244787, both of which are hereby incorporated by reference), operatively linked to the lac promoter under control of the lacl repressor. The expression vector also contains the P15a origin of replication and the chloramphenicol resistance gene. The resulting plasmids were transformed into E. coli W3110, using standard methods known in the art. The transformants were isolated by subjecting the cells to chloramphenicol selection, as known in the art (See e.g., U.S. Pat. No. 8,383,346 and WO2010/144103, both of which are hereby incorporated by reference).

### Example 2

### Preparation of HTP DNA Polymerase-Containing Wet Cell Pellets

E. coli cells containing recombinant DNA polymerase-encoding genes from monoclonal colonies were inoculated into 180 μl LB containing 1% glucose and 30 μg/mL CAM in the wells of 96-well, shallow-well microtiter plates. The plates were sealed with O₂-permeable seals, and cultures were grown overnight at 30° C., 200 rpm, and 85% humidity. Then, 10 μl of each of the cell cultures were transferred into the wells of 96-well, deep-well plates containing 390 μL TB and 30 μg/mL CAM. The deep-well plates were sealed with 02-permeable seals and incubated at 30° C., 250 rpm, and 85% humidity until OD₆₀₀ 0.6-0.8 was reached. The cell cultures were then induced with IPTG to a final concentration of 1 mM and incubated overnight under the same conditions as originally used. The cells were then pelleted using centrifugation at 4,000 rpm for 10 min. The supernatants were discarded, and the pellets were frozen at −80° C. prior to lysis.

### Example 3

### Preparation of HTP Polymerase-Containing Cell Lysates

First, 200 μl of lysis buffer containing 50 mM Tris-HCl pH 7.5 and 20 mM sodium chloride, were added to the cell paste in each well, produced as described in Example 2. The cells were shaken on a bench top shaker to resuspend. Resuspended cells were transferred to a 96 well hard shell plate and lysed at 80° C. for 60 min. in a thermocycler (MASTERCYLER® nexus gradient thermal cycler, Eppendorf). The plate was then centrifuged for 15 min at 4,000 rpm and 40° C. The clear supernatants were used in biocatalytic reactions to determine their activity, DNA sensitivity, thermostability and tolerance levels.

### Example 4

### Improvements Over SEQ ID NO: 2 in PCR Amplification Activity

SEQ ID NO: 2 was selected as the parent enzyme after screening various wild type polymerases for their activity in a PCR assay using beta-lactam fragment of the beta-lactamase gene cloned in pCK110900 vector (pCK-beta-lactamase) with primers SeqF1 (CCAATACGCAAACCGCCTC; SEQ ID NO: 3265) and seqR1 (CAACGGTGGTATATCCAGTGA; SEQ ID NO: 3266). In addition, their activities in the EVAEZ™ Flourometric Polymerase Activity Assay Kit (Biotium; cat. no. 29051) were also assessed.

### Example 5

Improvements Over SEQ ID NO: 2 in qPCR Enzyme Activity

SEQ ID NO: 2 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. The variants were then tested in the assay described below.

In this assay, a 15 μL reaction mixture that comprised 1500 copies of SARS-CoV2 DNA fragment Catalog #>CAT_102019, Twist, 500 nM N1 primers (SEQ ID NOS: 3262 and 3263), 125 nM FAM labeled probe (SEQ ID NO: 3264) (CDC EUA assay, nCoVEUA-01; IDT), 0.2 mM dNTPs (10 mM mixture from ThermoFisher), RT buffer (50 mM Tris-HCl, 73.1 mM KCl, 3.5 mM MgCl₂), and 5 vol % HTP lysate was prepared for each variant tested. Then, qPCR cycling (95° C. 2 min, 95° C. 3 sec, 55° C. 30 sec, for 45 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 5.1.

TABLE 5.1
Activity Relative to SEQ ID NO: 2

| | | FIOP qPCR |
|---|---|---|
| | | (Relative |
| SEQ ID NO: | Amino Acid Differences | to SEQ ID |
| (nt/aa) | (Relative to SEQ ID NO: 2) | NO: 2)¹ |
| 1/2 | | + |
| 3/4 | Q145H/S255V/P557R | ++++ |
| 5/6 | S255V/P391S/G398R/M476L/K602H/L658V/A660S/H685A | ++++ |
| 7/8 | Q145H/S255V/P391S/P557R/F758W | ++++ |
| 9/10 | Q50H/S255V/P557R/H685A | ++++ |
| 11/12 | Q145H/P391S/V472A/M476L/L658V/A660S/F758W | ++++ |
| 13/14 | Q145H/P391S/V472A/M476L/P557R/N743T/F758W | ++++ |
| 15/16 | P391S/M476L/G513T/N743T | ++++ |
| 17/18 | S255V/P391S/M476L/G513T/P557R/K602H/H685A/F758W | ++++ |
| 19/20 | S255V/G513T/P557R | ++++ |
| 21/22 | G398R/P557R/K602H/L658V | ++++ |
| 23/24 | Q145H/S255V/P391S/G398R/G513T/L658V/H685A/N743T | ++++ |
| 25/26 | S255V/V472A/F758W | ++++ |
| 27/28 | S255V/G398R/M476L | ++++ |
| 29/30 | S255V/P391S/V472A/L658V/A660S/F758W | ++++ |
| 31/32 | Q145H/H685A | ++++ |
| 33/34 | S255V/P391S/G398R/M476L/P557R | ++++ |
| 35/36 | S255V/P391S/V472A/G513T/F758W | ++++ |
| 37/38 | Q145H/F287L/L658V/F758W | ++++ |
| 39/40 | S255V/F287L/P391S/V472A/L658V/A660S/H685A | ++++ |
| 41/42 | Q145H/P391S/G513T/L658V/N743T/F758W | ++++ |
| 43/44 | S255V/K602H | ++++ |
| 45/46 | Q145H/S255V/G513T | ++++ |
| 47/48 | Q145H/P391S/V472A/M476L/L658V/H685A/F758W | ++++ |
| 49/50 | Q145H/S255V/P391S/V472A/P557R/H685A | ++++ |
| 51/52 | Q145H/P557R/H685A/F758W | ++++ |
| 53/54 | Q145H/P391S/G513T/P557R/L658V/H685A | ++++ |
| 55/56 | S255V/P391S/V472A/P557R/L658V/A660S/H685A/N743T/F758W | ++++ |
| 57/58 | F287L/H685A | ++++ |
| 59/60 | P391S/M476L/P557R | ++++ |
| 61/62 | P557R/N743T | ++++ |
| 63/64 | G11D/F287L | ++++ |
| 65/66 | P391S/G513T/L658V/A660S | ++++ |
| 67/68 | P391S/P557R/F758W | ++++ |
| 69/70 | G513T/P557R/F758W | ++++ |
| 71/72 | P391S/V472A/G513T/P557R/K602H/L658V | ++++ |
| 73/74 | Q145H/S255V/P391S/V472A/M476L/H685A/N743T | ++++ |
| 75/76 | Q145H/P391S/G398R/M476L/F758W | ++++ |
| 77/78 | S255V/P391S/G398R/G513T/P557R/L658V/N743T/F758W | ++++ |
| 79/80 | I161V/A235E/R645K/A699V | ++++ |
| 81/82 | V472A | ++++ |
| 83/84 | S255V/F287L/P391S/V472A/P557R/L658V/A660S | ++++ |
| 85/86 | R271H/V472A | ++++ |
| 87/88 | M476L/H685A | ++++ |
| 89/90 | Q145H/G513T/H685A | ++++ |
| 91/92 | A235E/R252L/G269R/R352K/M476I/R645K/I674V | ++++ |
| 93/94 | Q145H/F287L/P391S/M476L/G513T/P557R/K602H | +++ |
| 95/96 | F287L/P391S/P557R/H685A | +++ |
| 97/98 | R170L/A194V/A235E/R252L/G269R/P323S/G412A/A423Q/ | +++ |
| | M476I/K605R/R645K/A660P/I674V | +++ |
| 99/100 | I161V/R170L/A235E/R319V/G412A | +++ |
| 101/102 | R170L/A235E/R352K/A699V | +++ |
| 103/104 | I161V/A235E/R352K/A423Q/R645K/I674V | +++ |
| 105/106 | G513T | +++ |
| 107/108 | L658V | +++ |
| 109/110 | Q145H/F267S/P391S/G398R/G513T/P557R/L658V/H685A/N743T | +++ |
| 111/112 | P391S/G398R/M476L/H685A | +++ |
| 113/114 | Q145H/P391S/P557R/N743T | +++ |
| 115/116 | Q145H/S255V/F287L/P391S/V472A/M476L/P557R | +++ |
| 117/118 | I161V/A194V/R434K | +++ |
| 119/120 | M476L/P557R/F758W | +++ |
| 121/122 | S255V/P391S/K602H | +++ |
| 123/124 | Q145H/G398R/V472A/P557R/A660S | +++ |
| 125/126 | I161V/R352K | +++ |
| 127/128 | V472A/G513T/P557R/L658V/N743T/F758W | +++ |
| 129/130 | S255V/G513T/P557R/H685A/F758W | +++ |
| 131/132 | Q145H/S255V/P391S/G513T/P557R/N743T/F758W | +++ |
| 133/134 | Q145H/S255V/F287L/P391S/P557R/H685A/N743T/F758W | +++ |
| 135/136 | I161V/A235E/K249R/R319V/G412A/K605R/S659P | +++ |
| 137/138 | A194V/R252L/G269R/R319V/R434K/K605R/R645K/S659P/I674V/A699V | +++ |
| 139/140 | Q145H/S255V/P557R/L658V/A660S/H685A | +++ |
| 141/142 | A194V/A235E/R645K | +++ |
| 143/144 | S255V/P391S/P557R | +++ |
| 145/146 | V472A/P557R/A660S/H685A/N743T/F758W | +++ |
| 147/148 | S255V/P557R/L658V | +++ |
| 149/150 | A660S | +++ |
| 151/152 | P391S/H685A | +++ |
| 153/154 | Q145H/P391S/V472A/M476L/P557R/K602H/A660S/H685A/N743T | +++ |
| 155/156 | K602H | +++ |
| 157/158 | P557R/L658V/H685A | +++ |
| 159/160 | F287L/P391S/P557R | +++ |
| 161/162 | P197Q | +++ |
| 163/164 | P557R/L658V/A660S | +++ |
| 165/166 | I161V/A194V/R252L | +++ |
| 167/168 | V472A/M476L/P557R/L658V/F758W | +++ |
| 169/170 | A194V/R645K/S659P | +++ |
| 171/172 | I161V/R319V/A660P | +++ |
| 173/174 | R485K | +++ |
| 175/176 | R170L/A235E/R352K/I674V/A699V | +++ |
| 177/178 | A235E/K605R/R645K | +++ |
| 179/180 | R358N | +++ |
| 181/182 | Q145H/P391S/V472A/M476L/F758W | +++ |
| 183/184 | S255V/G398R/M476L/P557R/L658V/A660S/H685A | +++ |
| 185/186 | S659E | +++ |
| 187/188 | A235E/R252L/G269R/A423Q/K605R/R645K/A660P/I674V | +++ |
| 189/190 | Q145H/S255V/P391S/G398R/V472A/P557R/H685A | +++ |
| 191/192 | A235E/G412A/R645K | +++ |
| 193/194 | S255V/G398R/G513T/F758W | +++ |
| 195/196 | R352G | +++ |
| 197/198 | Q145H/S255V/P391S/G398R/M476L/P557R/F758W | +++ |
| 199/200 | R170L/A235E/K249R/R352K/R645K | +++ |
| 201/202 | R170L/A235E/G412A | +++ |
| 203/204 | P391S/L658V | +++ |
| 205/206 | Q145H/S255V/V472A/G513T/P557R/K602H/A660S/N743T | +++ |
| 207/208 | P391S/G398R/G513T/K602H/L658V/A660S/F758W | +++ |
| 209/210 | I161V/S162A/G269R/M476I/A660P | +++ |
| 211/212 | R170L/A235E/R645K | +++ |
| 213/214 | G513T/P557R/N743T | +++ |
| 215/216 | L331S | +++ |
| 217/218 | S255V/P391S/H685A/F758W | +++ |
| 219/220 | Q145H/P557R/L658V | +++ |
| 221/222 | S255V/P391S/M476L/P557R/K602H/A660S/F758W | +++ |
| 223/224 | I161V/S162A/S659P/A660P/I674V | +++ |
| 225/226 | A194V/A235E/K605R | +++ |
| 227/228 | S162A/A423Q/R645K | +++ |
| 229/230 | S255V/D260A/G398R/P557R | +++ |
| 231/232 | P377Q | +++ |
| 233/234 | V472A/G513T/P557R/A660S/H685A | +++ |
| 235/236 | A194V/A235E/R252L/R319V/A423Q | +++ |
| 237/238 | Q145H/S255V/F287L/P391S/M476L/G513T | +++ |
| 239/240 | E372N | +++ |
| 241/242 | A235E/G269R/P323S/G412A/R434K/S659P/I674V/A699V | +++ |
| 243/244 | R170L/A235E/A660P | +++ |
| 245/246 | A194V/A235E/R252L | +++ |
| 247/248 | I161V/R170L/A235E/G269R/R319V | +++ |
| 249/250 | Q145H/S255V/P391S/M476L/P557R/K602H/A660S/N743T | +++ |
| 251/252 | S255V/G398R/V472A/M476L/P557R/L658V | +++ |
| 253/254 | R704K | +++ |
| 255/256 | E329Q | +++ |
| 257/258 | S255V/P391S/M476L/P557R/L658V | +++ |
| 259/260 | K217T | +++ |
| 261/262 | I674V | +++ |
| 263/264 | R170L/A235E/R319V/R352K/G412A/R434K/M476I/R645K/A660P/I674V | +++ |
| 265/266 | G339E | +++ |
| 267/268 | K227E | +++ |
| 269/270 | R170L/G269R/R352K/A423Q/M476I/K605R/S659P/A660P/A699V | +++ |
| 271/272 | A235E | +++ |
| 273/274 | L110M | +++ |
| 275/276 | H29N | +++ |
| 277/278 | Q145H/F287L/P391S/V472A/P557R/K602H/L658V/A660S/H685A | +++ |
| 279/280 | F287L/P391S/V472A/M476L/P557R/K602H/L658V/A660S/H685A/N743T | +++ |
| 281/282 | A235E/R252L | +++ |
| 283/284 | R319V/R434K/M476I | +++ |
| 285/286 | I161V/K249R/G412A/M476I/K605R/R645K/S659P/A660P/I674V | +++ |
| 287/288 | L164C | +++ |
| 289/290 | K605R/A699V | +++ |
| 291/292 | A423Q/M476I/R645K/I674V | +++ |
| 293/294 | A194V/P323S/A699V | +++ |
| 295/296 | A235E/R319V/K605R/R645K/I674V | +++ |
| 297/298 | M476L/F758W | +++ |
| 299/300 | A235E/R352K/G412A/S659P/A660P/I674V/A699V | +++ |
| 301/302 | S162A/R170L/A194V/A235E/K249R/G269R/I674V/A699V | +++ |
| 303/304 | E533D | +++ |
| 305/306 | R268K | +++ |
| 307/308 | Q145H/P391S/G513T/F758W | +++ |
| 309/310 | R319V/R352K/G412A/R434K/A699V | +++ |
| 311/312 | L425F | +++ |
| 313/314 | I161V/S162A/A194V/G269R/K605R/A660P | +++ |
| 315/316 | R645K/S659P | +++ |
| 317/318 | A423V | +++ |
| 319/320 | R352K/K605R/S659P | +++ |
| 321/322 | Q145H/P391S/P557R/A660S/H685A | +++ |
| 323/324 | R170L/A235E/G269R/R434K/I674V | +++ |
| 325/326 | Q145H/S255V/P391S/M476L/F758W | +++ |
| 327/328 | F758W | +++ |
| 329/330 | I161V/A235E/R252L/R319V/R352K/R645K/I674V/A699V | +++ |
| 331/332 | Q145H/P391S | +++ |
| 333/334 | A423Q/R645K | +++ |
| 335/336 | A194V/R319V/R352K/K605R/A660P | +++ |
| 337/338 | A235E/M476I/I674V | +++ |
| 339/340 | A194V/S198G/A235E/R252L/P323S/G412A/K605R/R645K | +++ |
| 341/342 | M476L | +++ |
| 343/344 | R352K/G412A/K605R/R645K/A699V | +++ |
| 345/346 | L354I | +++ |
| 347/348 | K540N | +++ |
| 349/350 | A235E/K249R/R252L/R434K/K605R/R645K/A699V | +++ |
| 351/352 | I161V/A194V/R252L/G412A/K605R | +++ |
| 353/354 | A235E/R352K/K605R | +++ |
| 355/356 | S162A/A235E/G269R/A423Q/M476I/K605R/R645K/S659P | +++ |
| 357/358 | R170L/A194V/R319V/R352K/M476I/A699V | +++ |
| 359/360 | L297T | +++ |
| 361/362 | Q145H/S255V/G398R/V472A/M476L/G513T/P557R/L658V/A660S | +++ |
| 363/364 | S255V/P391S | +++ |
| 365/366 | Q145H/P391S/G513T/P557R/K602H | +++ |
| 367/368 | Q145H/P391S/V472A/M476L/G513T/P557R/L658V/H685A/F758W | +++ |
| 369/370 | I161V/A194V/S198G/A235E/R252L/A423Q/R434K/K605R/R645K/A699V | +++ |
| 371/372 | R319V | +++ |
| 373/374 | Q145H/S255V/V472A/M476L/P557R/H685A/N743T/F758W | +++ |
| 375/376 | I161V/A194V/R252L/P323S/R352K/G412A/K605R/S659P/A660P/I674V/A699V | +++ |
| 377/378 | A194V/R352K/K605R/R645K/A699V | +++ |
| 379/380 | P391S | +++ |
| 381/382 | R170L/A194V/A235E/R319V/G412A/M476I/A699V | +++ |
| 383/384 | K602H/H685A | +++ |
| 385/386 | L365I | +++ |
| 387/388 | P391S/K602H | +++ |
| 389/390 | P391S/A660S/N743T/F758W | +++ |
| 391/392 | S162A/R352K | +++ |
| 393/394 | R252L/R352K/R645K/A660P | +++ |
| 395/396 | Q145H/S255V/N743T/F758W | +++ |
| 397/398 | R170L/A235E/G269R/R352K | ++ |
| 399/400 | A235E/R352K | ++ |
| 401/402 | R478H/P557R | ++ |
| 403/404 | L361I | ++ |
| 405/406 | I161V/R170L/R252L/R352K/G412A/R434K/A699V | ++ |
| 407/408 | R645K/I674V/A699V | ++ |
| 409/410 | I161V/R170L/A235E/K249R/R252L/R434K | ++ |
| 411/412 | G398R/V472A | ++ |
| 413/414 | R170L/K249R/R252L/G269R/A423Q/S659P | ++ |
| 415/416 | S255V/F287L | ++ |
| 417/418 | A235E/R252L/G269R/R352K/R434K/K605R/I674V/A699V | ++ |
| 419/420 | L360F | ++ |
| 421/422 | P391S/A451V/V472A/G513T/P557R | ++ |
| 423/424 | I161V/R252L/R319V/R352K/G412A/A423Q/M476I/K605R/R645K | ++ |
| 425/426 | P391S/P557R | ++ |
| 427/428 | E440T | ++ |
| 429/430 | W175A | ++ |
| 431/432 | Q145H/P391S/P557R | ++ |
| 433/434 | W177F | ++ |
| 435/436 | P18H/Q145H/S255V | ++ |
| 437/438 | A350F | ++ |
| 439/440 | L426V | ++ |
| 441/442 | E356N | ++ |
| 443/444 | S162L | ++ |
| 445/446 | E253D | ++ |
| 447/448 | K249R/R252L/R319V/R352K/R645K | ++ |
| 449/450 | P396K | ++ |
| 451/452 | T379I | ++ |
| 453/454 | A194V/K249R/R252L/R645K/A699V | ++ |
| 455/456 | F281M | ++ |
| 457/458 | G412A/R645K | ++ |
| 459/460 | P202S/R268K | ++ |
| 461/462 | A235E/K249R/R252L/P323S/R352K/M476I | ++ |
| 463/464 | A194V/R434K | ++ |
| 465/466 | S255V/F287L/P391S/M476L/P557R | ++ |
| 467/468 | Q145H/F287L/P391S/V472A/K602H/L658V | ++ |
| 469/470 | R170L/G269R/M476I/R645K | ++ |
| 471/472 | V472A/F758W | ++ |
| 473/474 | S162A/G269R/R352K/G412A/M476I/K605R | ++ |
| 475/476 | Q145H/S255V/P391S/G398R/V472A/M476L/G513T/P557R/K602H/A660S | ++ |
| 477/478 | A235E/K249R/R252L/G412A/K605R/R645K/A699V | ++ |
| 479/480 | L435Y | ++ |
| 481/482 | R352K/K605R/R645K/A699V | ++ |
| 483/484 | S255V/P391S/V472A/P557R/L658V/A660S/F758W | ++ |
| 485/486 | S255V/P391S/M476L/F758W | ++ |
| 487/488 | G398T | ++ |
| 489/490 | F287L/P391S/P557R/K602H/L658V/N743T | ++ |
| 491/492 | L113F/R170L/A194V/A235E/K249R/R319V/R645K/S659P/A660P/I674V/A699V | ++ |
| 493/494 | I161V/S162A | ++ |
| 495/496 | R270Y | ++ |
| 497/498 | S255V/A660S | ++ |
| 499/500 | G412A | ++ |
| 501/502 | A194V/G269R/R319V/G412A/K605R/I674V/A699V | ++ |
| 503/504 | Q145H/M476L/P557R/A660S/H685A/N743T/F758W | ++ |
| 505/506 | I161V/R252L/R319V/I674V | ++ |
| 507/508 | Q145H/F287L/P391S/M476L/L658V/A660S/H685A | ++ |
| 509/510 | F287L/P391S/V472A | ++ |
| 511/512 | I161V/R170L/M476I | ++ |
| 513/514 | A455V | ++ |
| 515/516 | G269R/R352K | ++ |
| 517/518 | A810K | ++ |
| 519/520 | S162A | ++ |
| 521/522 | R170L/A194V/S198G/A235E/I674V/A699V | ++ |
| 523/524 | I161V/K605R | ++ |
| 525/526 | E506N | ++ |
| 527/528 | Q145H/P391S/P557R/L658V/F758W | ++ |
| 529/530 | K249R/G412A/M476I/K605R | ++ |
| 531/532 | G398R | ++ |
| 533/534 | A194V/A235E/A699V | ++ |
| 535/536 | A467N | ++ |
| 537/538 | K602H/Q703R | ++ |
| 539/540 | S255V/P391S/N743T | ++ |
| 541/542 | R645K | ++ |
| 543/544 | A423Q/A699V | ++ |
| 545/546 | R352K | ++ |
| 547/548 | V472A/G513T/L658V/A660S/N743T | ++ |
| 549/550 | L479M | ++ |
| 551/552 | M476I | ++ |
| 553/554 | G269R/K605R | ++ |
| 555/556 | R170L/G412A/A423Q/M476I/K605R/R645K/A660P/I674V | ++ |
| 557/558 | P564E | ++ |
| 559/560 | R532M | ++ |
| 561/562 | S162A/A194V/K249R/R645K/S659P | ++ |
| 563/564 | R170L | ++ |
| 565/566 | E635K | ++ |
| 567/568 | L658V/H685A/F758W | ++ |
| 569/570 | A194V | ++ |
| 571/572 | G508N | ++ |
| 573/574 | A235E/K249R/R252L/A423Q/A699V | ++ |
| 575/576 | L130M | ++ |
| 577/578 | A194V/S198G/R252L/R319V/A423Q/R645K/I674V | ++ |
| 579/580 | F287L/P391S/M476L/G513T | ++ |
| 581/582 | S255V/P391S/V472A/H685A | ++ |
| 583/584 | S198G/I674V/A699V | ++ |
| 585/586 | F287L/P391S/G513T/P557R/A660S/H685A/N743T/F758W | ++ |
| 587/588 | Q145H/S255V/G513T/K602H | ++ |
| 589/590 | L15S/G633N | ++ |
| 591/592 | S255V/F287L/P391S/L658V | ++ |
| 593/594 | L658V/A660S | ++ |
| 595/596 | G412A/I674V | ++ |
| 597/598 | S255V/P391S/G513T/K602H/L658V/A660S | ++ |
| 599/600 | R252L/R645K | ++ |
| 601/602 | K713Q | ++ |
| 603/604 | A423Q | ++ |
| 605/606 | R170L/A194V/G269R/R319V/G412A/A423Q/M476I/K605R/R645K | ++ |
| 607/608 | F267Y | ++ |
| 609/610 | A423Q/M476I/K605R | ++ |
| 611/612 | R352K/R645K | ++ |
| 613/614 | A194V/R252L/R319V/G412A/K605R | ++ |
| 615/616 | R170L/K249R/R252L/G269R/R352K/G412A/A423Q/R434K/M476I/S659P | ++ |
| 617/618 | S162A/R170L/G412A/R434K/M476I/K605R | ++ |
| 619/620 | R170L/M476I/I674V | ++ |
| 621/622 | R322V | ++ |
| 623/624 | V472A/M476L/G513T/N743T | ++ |
| 625/626 | P557R | ++ |
| 627/628 | G269R/R352K/G412A | ++ |
| 629/630 | Q145H/S255V/P391S/M476L/P557R/L658V/A660S/N743T | ++ |
| 631/632 | I161V/G269R/R434K/A699V | ++ |
| 633/634 | G20C/K602H | ++ |
| 635/636 | R170L/R252L/R352K/A423Q/I674V/A699V | ++ |
| 637/638 | K249R/R252L/R319V/K605R/A660P | ++ |
| 639/640 | I161V/A194V/A235E/K249R/R352K/G412A/R434K/I674V/A699V | ++ |
| 641/642 | K718D | ++ |
| 643/644 | P391S/K602H/F758W | ++ |
| 645/646 | P391S/V472A/M476L/P557R/K602H | ++ |
| 647/648 | A194V/R352K/M476I/S659P | ++ |
| 649/650 | T456K | ++ |
| 651/652 | S162A/K605R/R645K | ++ |
| 653/654 | E643H | ++ |
| 655/656 | S162A/R252L/R352K/G412A/R434K/R645K/A660P/A699V | ++ |
| 657/658 | R170L/K249R/G412A | + |
| 659/660 | I161V/S162A/R170L/A235E/K249R/R252L | + |
| 661/662 | P347I | + |
| 663/664 | I161V/A423Q/R645K | + |
| 665/666 | F287L/P391S/K602H | + |
| 667/668 | G168V | + |
| 669/670 | K279I | + |
| 671/672 | A194V/K249R/G412A/M476I/K605R/R645K/A699V | + |
| 673/674 | P391S/V472A/G513T/K602H/L658V | + |
| 675/676 | I161V/S162A/R352K/R434K/K605R | + |
| 677/678 | Q145H | + |
| 679/680 | K249R/R252L/G412A/A423Q/R645K/I674V/A699V | + |
| 681/682 | A235E/G269R/I674V/A699V | + |
| 683/684 | I161V/S162A/R170L/A194V/A235E/A423Q/A699V | + |

¹Levels of increased activity were determined relative to the reference polypeptide of SEQ ID NO: 2, and defined as follows: “+” > 0.85; “++” > 1.15; “+++” > 5; and “++++” > 15.

### Example 6

### Improvements Over SEQ ID NO: 2 in RT-qPCR Enzyme Activity

SEQ ID NO: 2 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. The variants were tested in the assay described below.

For each variant tested, a 15 μL reaction mixture comprising 1250 copies of SARS-CoV2 RNA fragment (Catalog #>102019, Twist, 500 nM N1 primers (SEQ ID NOS: 3262 and 3263), 125 nM FAM labeled probe (SEQ ID NO: 3264) CDC EUA assay, Catalog #2019-nCoVEUA-01; IDT), 0.2 mM dNTPs (10 mM mixture from ThermoFisher), RT buffer (50 mM Tris-HCl, 73.1 mM KCl, 3.5 mM MgCl₂), 1.25 ng/uL of CODEX® HiTemp reverse transcriptase (Codexis), and 5 vol % HTP lysate containing the variant, was used in a RT-qPCR cycling reaction (50° C. 15 min, 95° C. 2 min, 95° C. 5 sec, 60° C. 30 sec, for 50 cycles) in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 6.1.

TABLE 6.1
Activity Relative to SEQ ID NO: 2

| | | FIOP RT-qPCR |
|---|---|---|
| | | (Relative |
| SEQ ID NO: | Amino Acid Differences | to SEQ ID |
| (nt/aa) | (Relative to SEQ ID NO: 2) | NO: 2)¹ |
| 1/2 | | + |
| 251/252 | S255V/G398R/V472A/M476L/P557R/L658V | ++++ |
| 157/158 | P557R/L658V/H685A | ++++ |
| 685/686 | N743T | ++++ |
| 219/220 | Q145H/P557R/L658V | ++++ |
| 373/374 | Q145H/S255V/V472A/M476L/P557R/H685A/N743T/F758W | ++++ |
| 139/140 | Q145H/S255V/P557R/L658V/A660S/H685A | ++++ |
| 63/64 | G11D/F287L | ++++ |
| 537/538 | K602H/Q703R | ++++ |
| 9/10 | Q50H/S255V/P557R/H685A | ++++ |
| 645/646 | P391S/V472A/M476L/P557R/K602H | ++++ |
| 257/258 | S255V/P391S/M476L/P557R/L658V | ++++ |
| 421/422 | P391S/A451V/V472A/G513T/P557R | +++ |
| 687/688 | P391S/G581D | +++ |
| 515/516 | G269R/R352K | +++ |
| 49/50 | Q145H/S255V/P391S/V472A/P557R/H685A | +++ |
| 689/690 | Q145H/N743T | +++ |
| 367/368 | Q145H/P391S/V472A/M476L/G513T/P557R/L658V/H685A/F758W | +++ |
| 691/692 | S255V/P391S/V472A/P557R/A660S/N743T | +++ |
| 693/694 | K602H/F701L | +++ |
| 115/116 | Q145H/S255V/F287L/P391S/V472A/M476L/P557R | +++ |
| 183/184 | S255V/G398R/M476L/P557R/L658V/A660S/H685A | +++ |
| 401/402 | R478H/P557R | +++ |
| 637/638 | K249R/R252L/R319V/K605R/A660P | +++ |
| 599/600 | R252L/R645K | +++ |
| 163/164 | P557R/L658V/A660S | +++ |
| 569/570 | A194V | +++ |
| 627/628 | G269R/R352K/G412A | +++ |
| 563/564 | R170L | +++ |
| 695/696 | A699V | +++ |
| 697/698 | S255V/P391S/P557R | ++ |
| 573/574 | A235E/K249R/R252L/A423Q/A699V | ++ |
| 365/366 | Q145H/P391S/G513T/P557R/K602H | ++ |
| 499/500 | G412A | ++ |
| 123/124 | Q145H/G398R/V472A/P557R/A660S | ++ |
| 111/112 | P391S/G398R/M476L/H685A | ++ |
| 269/270 | R170L/G269R/R352K/A423Q/M476I/K605R/S659P/A660P/A699V | ++ |
| 639/640 | I161V/A194V/A235E/K249R/R352K/G412A/R434K/I674V/A699V | ++ |
| 291/292 | A423Q/M476I/R645K/I674V | ++ |
| 285/286 | I161V/K249R/G412A/M476I/K605R/R645K/S659P/A660P/I674V | ++ |
| 17/18 | S255V/P391S/M476L/G513T/P557R/K602H/H685A/F758W | ++ |
| 351/352 | I161V/A194V/R252L/G412A/K605R | ++ |
| 595/596 | G412A/I674V | ++ |
| 97/98 | R170L/A194V/A235E/R252L/G269R/P323S/G412A/ | ++ |
| | A423Q/M476I/K605R/R645K/A660P/I674V | |
| 391/392 | S162A/R352K | ++ |
| 553/554 | G269R/K605R | ++ |
| 425/426 | P391S/P557R | ++ |
| 371/372 | R319V | ++ |
| 241/242 | A235E/G269R/P323S/G412A/R434K/S659P/I674V/A699V | ++ |
| 101/102 | R170L/A235E/R352K/A699V | ++ |
| 343/344 | R352K/G412A/K605R/R645K/A699V | ++ |
| 107/108 | L658V | ++ |
| 211/212 | R170L/A235E/R645K | ++ |
| 699/700 | E506N | ++ |
| 701/702 | P557R | ++ |
| 545/546 | R352K | ++ |
| 177/178 | A235E/K605R/R645K | ++ |
| 201/202 | R170L/A235E/G412A | ++ |
| 149/150 | A660S | ++ |
| 501/502 | A194V/G269R/R319V/G412A/K605R/I674V/A699V | ++ |
| 265/266 | G339E | ++ |
| 519/520 | S162A | ++ |
| 137/138 | A194V/R252L/G269R/R319V/R434K/K605R/R645K/ | ++ |
| | S659P/I674V/A699V | |
| 209/210 | I161V/S162A/G269R/M476I/A660P | ++ |
| 703/704 | S255V | ++ |
| 125/126 | I161V/R352K | ++ |
| 113/114 | Q145H/P391S/P557R/N743T | ++ |
| 179/180 | R358N | ++ |
| 79/80 | I161V/A235E/R645K/A699V | ++ |
| 461/462 | A235E/K249R/R252L/P323S/R352K/M476I | ++ |
| 215/216 | L331S | ++ |
| 347/348 | K540N | + |
| 705/706 | K713Q | + |
| 305/306 | R268K | + |
| 635/636 | R170L/R252L/R352K/A423Q/I674V/A699V | + |
| 423/424 | I161V/R252L/R319V/R352K/G412A/A423Q/M476I/K605R/R645K | + |
| 169/170 | A194V/R645K/S659P | + |
| 263/264 | R170L/A235E/R319V/R352K/G412A/R434K/M476I/R645K/A660P/I674V | + |
| 309/310 | R319V/R352K/G412A/R434K/A699V | + |
| 333/334 | A423Q/R645K | + |
| 161/162 | P197Q | + |
| 227/228 | S162A/A423Q/R645K | + |
| 259/260 | K217T | + |
| 199/200 | R170L/A235E/K249R/R352K/R645K | + |
| 329/330 | I161V/A235E/R252L/R319V/R352K/R645K/I674V/A699V | + |
| 267/268 | K227E | + |
| 247/248 | I161V/R170L/A235E/G269R/R319V | + |
| 707/708 | L360F | + |
| 335/336 | A194V/R319V/R352K/K605R/A660P | + |
| 709/710 | P391S/V472A/M476L/P557R/L658V/A660S/N743T/F758W | + |
| 399/400 | A235E/R352K | + |
| 243/244 | R170L/A235E/A660P | + |
| 281/282 | A235E/R252L | + |
| 353/354 | A235E/R352K/K605R | + |
| 711/712 | S255V/M476L | + |
| 223/224 | I161V/S162A/S659P/A660P/I674V | + |
| 377/378 | A194V/R352K/K605R/R645K/A699V | + |
| 283/284 | R319V/R434K/M476I | + |
| 577/578 | A194V/S198G/R252L/R319V/A423Q/R645K/I674V | + |
| 713/714 | A467N | + |
| 593/594 | L658V/A660S | + |
| 275/276 | H29N | + |
| 345/346 | L354I | + |
| 187/188 | A235E/R252L/G269R/A423Q/K605R/R645K/A660P/I674V | + |
| 195/196 | R352G | + |
| 171/172 | I161V/R319V/A660P | + |
| 245/246 | A194V/A235E/R252L | + |
| 99/100 | I161V/R170L/A235E/R319V/G412A | + |
| 255/256 | E329Q | + |
| 175/176 | R170L/A235E/R352K/I674V/A699V | + |
| 457/458 | G412A/R645K | + |
| 715/716 | Q145H/S255V/P391S/H685A | + |
| 717/718 | P391S | + |
| 289/290 | K605R/A699V | + |
| 497/498 | S255V/A660S | + |

¹Levels of increased activity were determined relative to the reference polypeptide of SEQ ID NO: 2, and defined as follows: “+” > 0.85; “++” > 1.15; “+++” > 1.5; and “++++” > 2.

### Example 7

Improvements over SEQ ID NO: 178 in RT/qPCR Enzyme Activity

SEQ ID NO: 178 was selected as the parent enzyme for the round of directed evolution ane enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a qPCR assay, as described below.

For each variant tested, 15 μL reaction mixture comprising 1500 copies of SARS-CoV2 DNA fragment (Catalog #>102019, Twist), 500 nM N1 primers (SEQ ID NOS: 3262 and 3263), 125 nM FAM labeled probe SEQ ID NO: 3264) (CDC EUA assay, 2019-nCoV EUA-01; IDT), 0.2 mM dNTPs (10 mM mixture from ThermoFisher), RT buffer (50 mM Tris-HCl, 73.1 mM KCl, 3.5 mM MgCl₂), 2.5 vol % HTP lysate was prepared. Then, qPCR cycling (95° C. 2 min, 95° C. 3 sec, 55° C. 30 sec, for 45 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 7.1.

TABLE 7.1 Activity Relative to SEQ ID NO: 178

| | | FIOP qPCR |
|---|---|---|
| | | (Relative |
| SEQ ID NO: | Amino Acid Differences | to SEQ ID |
| (nt/aa) | (Relative to SEQ ID NO: 178) | NO: 178)¹ |
| 177/178 | | + |
| 719/720 | Q689N | +++ |
| 721/722 | K565Y | +++ |
| 723/724 | A423Q/M476L/P557R/S659P/A660S/H685A/N743T | +++ |
| 725/726 | R485A | +++ |
| 727/728 | A194V/A423Q/H685A | +++ |
| 729/730 | R170L/S659P | +++ |
| 731/732 | R170L/A194V/M476L/P557R/S659P | +++ |
| 733/734 | R170L/A423Q/P557R/L658V/S659P | +++ |
| 735/736 | S659P/A660S | +++ |
| 737/738 | S659P/A660S/H685A/N743T | +++ |
| 739/740 | A194V/A423Q/M476L/L658V/A660S/H685A | +++ |
| 741/742 | M476L/L658V/A660S/N743T | +++ |
| 743/744 | M476L/P557R/S659P | +++ |
| 745/746 | R170L/M476L/P557R/N743T | +++ |
| 747/748 | R170L/A423Q/P557R/L658V/N743T | +++ |
| 749/750 | R170L/A423Q/M476L | +++ |
| 751/752 | R170L/A194V/L658V/S659P/A660S/H685A/N743T | +++ |
| 753/754 | R170L/A423Q | +++ |
| 755/756 | H489Q/P557R/N743T | +++ |
| 757/758 | R170L/M476L/S659P | +++ |
| 759/760 | R170L/A423Q/P557R/L658V/A660S/N743T | +++ |
| 761/762 | R170L/A194V/A423Q/P557R/L658V/N743T | +++ |
| 763/764 | R170L/L658V/S659P/N743T | +++ |
| 765/766 | A423Q/M476L | +++ |
| 767/768 | A194V/P557R | +++ |
| 769/770 | A423Q/M476L/P557R/L658V/S659P/H685A/N743T | +++ |
| 771/772 | V534A/A670V | +++ |
| 773/774 | A423Q | +++ |
| 775/776 | P557R | +++ |
| 777/778 | R170L/A194V/L658V/N743T | +++ |
| 779/780 | A423Q/P557R/L658V/S659P/A660S/H685A | +++ |
| 781/782 | A423Q/M476L/L658V/H685A/N743T | +++ |
| 783/784 | A423Q/L658V/S659P/A660S | ++ |
| 785/786 | W275M | ++ |
| 787/788 | P557R/L658V | ++ |
| 789/790 | P557R/L658V/S659P | ++ |
| 791/792 | M476L/P557R/L658V/A660S/H685A | ++ |
| 793/794 | A194V/A423Q/P557R/L658V/S659P/N743T | ++ |
| 795/796 | R170L/A423Q/M476L/L658V/H685A | ++ |
| 797/798 | M476L/L658V/S659P/A660S/H685A/N743T | ++ |
| 799/800 | R170L/A423Q/L658V/S659P/N743T | ++ |
| 801/802 | L658V/N743T | ++ |
| 803/804 | M12E | ++ |
| 805/806 | R170L/A194V/A423Q/P557R/L658V/S659P/N743T | ++ |
| 807/808 | L658V/S659P | ++ |
| 809/810 | P557R/N743T | ++ |
| 811/812 | R170L/L658V | ++ |
| 813/814 | A423Q/P557R/L658V/A660S/N743T | ++ |
| 815/816 | R170L/A423Q/A451H/P557R/L658V/S659P/H685A/N743T | ++ |
| 817/818 | E209W | ++ |
| 819/820 | R170L/P557R/L658V/A660S/H685A/L687F | ++ |
| 821/822 | R170L/A423Q/L658V/A660S | ++ |
| 823/824 | A194V/L658V/S659P/H685A | ++ |
| 825/826 | A194V/P557R/L658V/S659P/H685A | ++ |
| 827/828 | A194V/A451H/M476L/P557R/L658V/S659P | ++ |
| 829/830 | R170L/A423Q/P557R/S659P/A660S/H685A/N743T | ++ |
| 831/832 | M476L/P557R/L658V/S659P | ++ |
| 833/834 | A423Q/P557R/L658V | ++ |
| 835/836 | M476L | ++ |
| 837/838 | R170L/A423Q/P557R/H685A/N743T | ++ |
| 839/840 | L658V/S659P/A660S/H685A/N743T | ++ |
| 841/842 | R170L/A423Q/M476L/L658V/A660S/H685A/N743T | ++ |
| 843/844 | E209V | ++ |
| 845/846 | K517H | ++ |
| 847/848 | R170L/A194V/P557R | ++ |
| 849/850 | R170L/L658V/S659P | ++ |
| 851/852 | A423Q/N743T | ++ |
| 853/854 | R170L/A194V/A451H/M476L/P557R/L658V/S659P/A660S/N743T | ++ |
| 855/856 | R170L/P557R | ++ |
| 857/858 | R170L/A423Q/P557R/H685A | ++ |
| 859/860 | A194V/L658V/A660S | ++ |
| 861/862 | E264S | ++ |
| 863/864 | A423Q/M476L/P557R/L658V/S659P/A660S | ++ |
| 865/866 | R170L/M476L/P557R/L658V/H685A | ++ |
| 867/868 | A525V/P557R/S659P/A660S/N743T | ++ |
| 869/870 | R170L/A660S | ++ |
| 871/872 | A423Q/M476L/L658V/N743T | ++ |
| 873/874 | R170L/M476L/P557R/L658V/S659P | ++ |
| 875/876 | P511L | ++ |
| 877/878 | R170L/A423Q/M476L/H685A/N743T | ++ |
| 879/880 | R170L/A194V/A423Q/P557R/L658V/A660S/H685A | ++ |
| 881/882 | L658V/S659P/H685A | ++ |
| 883/884 | R170L/A423Q/M476L/L658V/S659P/A660S/H685A/N743T | ++ |
| 885/886 | A423Q/M476L/P557R/S659P/A660S/N743T | ++ |
| 887/888 | A423Q/A451H/P557R/L658V/H685A | ++ |
| 889/890 | A423Q/A451H/P557R/L658V | ++ |
| 891/892 | P122A | ++ |
| 893/894 | H685A | ++ |
| 895/896 | R170L/A423Q/M476L/L658V/S659P | ++ |
| 897/898 | A194V/A451H/M476L/P557R/L658V/S659P/A660S/N743T | ++ |
| 899/900 | A194V/A423Q/A451H/P557R/L658V/S659P/N743T | ++ |
| 901/902 | R170L/A194V | ++ |
| 903/904 | K39P | ++ |
| 905/906 | A423Q/P557R/H685A | ++ |
| 907/908 | R170L/A423Q/N743T | ++ |
| 909/910 | W275E | ++ |
| 911/912 | R170L/A423Q/L658V | ++ |
| 913/914 | K517T | ++ |
| 915/916 | A423Q/H685A | ++ |
| 917/918 | R170L/A423Q/A451H/P557R | ++ |
| 919/920 | A423Q/M476L/L658V/A660S/N743T | ++ |
| 921/922 | A423Q/A451H/M476L/P557R/L658V/H685A | ++ |
| 923/924 | R170L/A423Q/M476L/H685A | ++ |
| 925/926 | V534A | ++ |
| 927/928 | P557R/L658V/S659P/A660S | ++ |
| 929/930 | M476L/P557R/S659P/A660S | ++ |
| 931/932 | A194V/P557R/S659P/N743T | ++ |
| 933/934 | N743T | ++ |
| 935/936 | R170L/A423Q/A451H/M476L/P557R/L658V/S659P/N743T | ++ |
| 937/938 | R170L/A194V/A451H/M476L/P557R/L658V | ++ |
| 939/940 | S198G/A423Q/M476L/P557R/N743T | ++ |
| 941/942 | M12V | ++ |
| 943/944 | A423Q/L658V | ++ |
| 945/946 | R170L/A423Q/P557R/A660S/N743T | ++ |
| 947/948 | K514V | ++ |
| 949/950 | L301G | ++ |
| 951/952 | R596S | ++ |
| 953/954 | S198G | ++ |
| 955/956 | K517A | ++ |
| 957/958 | A423Q/P557R/H685A/N743T | ++ |
| 959/960 | K205R | ++ |
| 961/962 | L470E | ++ |
| 963/964 | R170L/A451H/P557R/L658V/A660S | ++ |
| 965/966 | R170L/A194V/L658V | ++ |
| 967/968 | E783H | ++ |
| 969/970 | A423Q/P557R/L658V/S659P/A660S | ++ |
| 971/972 | A423Q/A660S | ++ |
| 973/974 | R45S | ++ |
| 975/976 | P557R/S659P/A660S | ++ |
| 977/978 | A423Q/L658V/A660S | ++ |
| 979/980 | R45L | ++ |
| 981/982 | R170L/A194V/A451H/M476L/P557R/L658V/H685A | ++ |
| 983/984 | K549V | ++ |
| 985/986 | R170L/A423Q/M476L/L658V/H685A/N743T | ++ |
| 987/988 | Q707R | ++ |
| 989/990 | K355G | ++ |
| 991/992 | Y695K | ++ |
| 993/994 | N492Y | ++ |
| 995/996 | K565R | ++ |
| 997/998 | R139D/T395A | ++ |
| 999/1000 | G412A/I674V | ++ |
| 1001/1002 | Y695Q | ++ |
| 1003/1004 | R170L/A423Q/A451H/P557R/S659P | ++ |
| 1005/1006 | R139S | ++ |
| 1007/1008 | N492R | ++ |
| 1009/1010 | E345P | ++ |
| 1011/1012 | L222I | ++ |
| 1013/1014 | L470V | ++ |
| 1015/1016 | G412A/A660P | ++ |
| 1017/1018 | R252L/L331S | ++ |
| 1019/1020 | L15V | ++ |
| 1021/1022 | E696Q | ++ |
| 1023/1024 | A423Q/A451H/P557R | ++ |
| 1025/1026 | G412A/A467N/A660P/I674V/A699V | ++ |
| 1027/1028 | R252L/R704K | ++ |
| 1029/1030 | Q689D | ++ |
| 1031/1032 | E433V | ++ |
| 1033/1034 | L658V | ++ |
| 1035/1036 | K19S | ++ |
| 1037/1038 | L331S/I674V | ++ |
| 1039/1040 | G269R | ++ |
| 1041/1042 | E433S | ++ |
| 1043/1044 | P14V | ++ |
| 1045/1046 | R478G | ++ |
| 1047/1048 | P598G | ++ |
| 1049/1050 | N492M | ++ |
| 1051/1052 | R485E | ++ |
| 1053/1054 | L331S/G412A/A467N/I674V/R704K | ++ |
| 1055/1056 | Y86G | ++ |
| 1057/1058 | P95D | ++ |
| 1059/1060 | E696A | ++ |
| 1061/1062 | R252L/L331S/R352K/G412A/E413K/A467N/A660P/R704K | ++ |
| 1063/1064 | R252L/L331S/A467N/A660P/I674V | ++ |
| 1065/1066 | R252L/L331S/G412A/E506N/I674V | ++ |
| 1067/1068 | G269R/L331S/A699V/R704K | ++ |
| 1069/1070 | R252L/G269R/L331S/G339E/G412A/E413K | ++ |
| 1071/1072 | R501K | ++ |
| 1073/1074 | M476L/E480K/P557R/S659P | ++ |
| 1075/1076 | I674V | ++ |
| 1077/1078 | A423Q/H685A/N743T | ++ |
| 1079/1080 | K39E | ++ |
| 1081/1082 | K249E | ++ |
| 1083/1084 | G269R/G412A/A467N/R704K | ++ |
| 1085/1086 | L331S/E506N | ++ |
| 1087/1088 | L331S/R704K | ++ |
| 1089/1090 | L331S/E413K/A467N/A660P/I674V | ++ |
| 1091/1092 | L331S/R352K/R358N/A699V/R704K | ++ |
| 1093/1094 | L331S/R358N | ++ |
| 1095/1096 | R352K/I674V | ++ |
| 1097/1098 | R252L | ++ |
| 1099/1100 | E209P | ++ |
| 1101/1102 | P821S | ++ |
| 1103/1104 | R252L/G269R/G339E/R352K/E356G/A660P/R704K | ++ |
| 1105/1106 | R252L/R358N/I674V | ++ |
| 1107/1108 | A467N/L658V/A660S/I674V/A699V | ++ |
| 1109/1110 | A699V | ++ |
| 1111/1112 | R268H/P510A | ++ |
| 1113/1114 | R252L/A699V | ++ |
| 1115/1116 | R485V | ++ |
| 1117/1118 | R252L/G339E/G412A/E413K/A467N/I674V | ++ |
| 1119/1120 | R352K/G412A/E413K/A467N/E506N/A699V | ++ |
| 1121/1122 | E264A | ++ |
| 1123/1124 | E433P/D497N | ++ |
| 1125/1126 | G412A/A467N/E506N/I674V/R704K | + |
| 1127/1128 | L331S | + |
| 1129/1130 | G412A/I674V/A699V | + |
| 1131/1132 | R170L/A194V/A451H/P557R/S659P/A660S | + |
| 1133/1134 | R252L/G339E/R352K/I674V/R704K | + |
| 1135/1136 | P598K | + |
| 1137/1138 | G269R/R352K/A467N/E506N/I674V | + |
| 1139/1140 | R252L/R258H/L331S/R352K/A467N/E506N/I674V | + |
| 1141/1142 | G269R/L331S/R352K/A467N/E506N/A699V | + |
| 1143/1144 | K565S | + |
| 1145/1146 | R252L/I674V | + |
| 1147/1148 | G269R/A699V/R704K | + |
| 1149/1150 | R252L/L331S/G339E/R352K/A467N/E506N | + |
| 1151/1152 | G339E/A467N/I674V | + |
| 1153/1154 | K205S | + |
| 1155/1156 | R252L/E413K/E506N/A660P/R704K | + |
| 1157/1158 | E87P | + |
| 1159/1160 | Y695T | + |
| 1161/1162 | E413K/E506N | + |
| 1163/1164 | G269R/L331S/A660P/A699V | + |
| 1165/1166 | K517M | + |
| 1167/1168 | A423Q/A451H/M476L/L658V/H685A | + |
| 1169/1170 | R252L/E413K | + |
| 1171/1172 | G339E/G412A/E413K | + |
| 1173/1174 | K520V | + |
| 1175/1176 | G269R/L331S/I674V/A699V | + |
| 1177/1178 | R477E | + |
| 1179/1180 | R268A/S621N | + |
| 1181/1182 | R252L/L331S/A660P | + |
| 1183/1184 | N228S | + |
| 1185/1186 | R252L/R352K/G412A | + |
| 1187/1188 | Q707A | + |
| 1189/1190 | R252L/G412A/A467N/A660P/A699V/R704K | + |
| 1191/1192 | P557R/A660S | + |
| 1193/1194 | G269R/L331S/G412A/E413K/A467N/E506N/A660P/I674V | + |
| 1195/1196 | S44L | + |
| 1197/1198 | V341A/R352K/R704K | + |
| 1199/1200 | A699V/R704K | + |
| 1201/1202 | R745P | + |
| 1203/1204 | R478S | + |
| 1205/1206 | P197K | + |
| 1207/1208 | E167D | + |
| 1209/1210 | S198P | + |
| 1211/1212 | K747V | + |
| 1213/1214 | E696K | + |
| 1215/1216 | R478L | + |
| 1217/1218 | R252L/G269R/L331S/R352K/G412A/I674V/R704K | + |
| 1219/1220 | L331S/G412A/A660P/A699V | + |
| 1221/1222 | K517R | + |
| 1223/1224 | R252L/G269R/G412A/I674V | + |
| 1225/1226 | E506N/I674V/R704K | + |
| 1227/1228 | R252L/G269R | + |
| 1229/1230 | E17S | + |
| 1231/1232 | G269R/L331S/R352K/G412A/A467N/E506N/R704K | + |
| 1233/1234 | G339E/E506N | + |
| 1235/1236 | G339E/R352K/G412A/A660P/I674V | + |
| 1237/1238 | L301T | + |
| 1239/1240 | R485G | + |
| 1241/1242 | G412A | + |
| 1243/1244 | E433R | + |
| 1245/1246 | L331S/G412A | + |
| 1247/1248 | G339E/E506N/A699V | + |
| 1249/1250 | G269R/L331S/A699V | + |
| 1251/1252 | G269R/L331S/E413K/E506N | + |
| 1253/1254 | L331S/R352K/G412A/E413K/E506N | + |
| 1255/1256 | G339E/R352K/I674V/A699V | + |
| 1257/1258 | L331S/G412A/A467N/R704K | + |
| 1259/1260 | L331S/A660P/I674V/A699V | + |
| 1261/1262 | R252L/G339E/G412A/A467N/I674V | + |
| 1263/1264 | L331S/G339E/G412A/A467N/E506N/R704K | + |
| 1265/1266 | R252L/G269R/L331S/E506N/I674V | + |
| 1267/1268 | R252L/G339E/G412A/I674V/R704K | + |
| 1269/1270 | E413K/A467N | + |
| 1271/1272 | G269R/I674V | + |
| 1273/1274 | R252L/L331S/E413K | + |
| 1275/1276 | E533L | + |
| 1277/1278 | R252L/L331S/G412A/E413K/I674V | + |
| 1279/1280 | R252L/L331S/A699V | + |
| 1281/1282 | R252L/L331S/R352K/G412A/E506N/I674V | + |
| 1283/1284 | P14E | + |
| 1285/1286 | E413K/E506N/R704K | + |
| 1287/1288 | G269R/L331S/A467N/E506N/A660P | + |
| 1289/1290 | E433K | + |
| 1291/1292 | R252L/G269R/I674V | + |
| 1293/1294 | G412A/E413K/A467N/I674V | + |
| 1295/1296 | G269R/G412A/E413K/E506N/R704K | + |
| 1297/1298 | G339E | + |
| 1299/1300 | T725H | + |
| 1301/1302 | R252L/V341A/G412A/I674V | + |
| 1303/1304 | E696G | + |
| 1305/1306 | P323Q | + |
| 1307/1308 | K179M | + |
| 1309/1310 | R252L/E506N/R704K | + |
| 1311/1312 | M12S | + |
| 1313/1314 | F36H | + |
| 1315/1316 | R252L/G339E/A467N/E506N/A699V/R704K | + |
| 1317/1318 | G269R/A467N/E506N | + |
| 1319/1320 | R252L/A467N/E506N/I674V/R704K | + |
| 1321/1322 | R252L/L331S/G412A/A467N/E506N/A699V/R704K | + |
| 1323/1324 | G269R/L331S/R352K/A467N/A699V | + |
| 1325/1326 | S44A | + |
| 1327/1328 | L331S/A467N/I674V/A699V | + |
| 1329/1330 | E506N/A660P | + |
| 1331/1332 | R352K/E413K/I674V | + |
| 1333/1334 | E209R | + |
| 1335/1336 | Q642E | + |
| 1337/1338 | R252L/G412A/A467N | + |
| 1339/1340 | R252L/R352K/E506N | + |
| 1341/1342 | G339E/A660P | + |
| 1343/1344 | K39V | + |
| 1345/1346 | R252L/V341A/G412A/E413K | + |
| 1347/1348 | E87A | + |
| 1349/1350 | R358N/G412A | + |
| 1351/1352 | L331S/I674V/A699V | + |
| 1353/1354 | P598R | + |
| 1355/1356 | S198V | + |
| 1357/1358 | R428G | + |
| 1359/1360 | R252L/A467N | + |
| 1361/1362 | R252L/G269R/A467N/E506N | + |
| 1363/1364 | E433G | + |
| 1365/1366 | R478I | + |
| 1367/1368 | G339E/R352K/G412A/A467N/A660P | + |
| 1369/1370 | R428C | + |
| 1371/1372 | R82K | + |
| 1373/1374 | G269R/R358N/A467N/E506N/A660P/A699V | + |
| 1375/1376 | M12L | + |
| 1377/1378 | K214D | + |
| 1379/1380 | R252L/R258H/L331S | + |
| 1381/1382 | L331S/G412A/E413K/A467N/A699V/R704K | + |
| 1383/1384 | R477T | + |
| 1385/1386 | R252L/R352K/A660P | + |
| 1387/1388 | R252L/G269R/E413K/A467N/A660P/A699V/R704K | + |
| 1389/1390 | A467N/E506N/A699V/R704K | + |
| 1391/1392 | R252L/E413K/A699V | + |
| 1393/1394 | G269R/R358N/G412A/A660P/R704K | + |
| 1395/1396 | K214R | + |
| 1397/1398 | E506N/I674V/A699V/R704K | + |
| 1399/1400 | R501L | + |
| 1401/1402 | G412A/E413K/A467N/A660P | + |
| 1403/1404 | R252L/G339E/G412A/E413K/I674V/A699V/R704K | + |
| 1405/1406 | R252L/L331S/G412A/E413K | + |
| 1407/1408 | R485I | + |
| 1409/1410 | I674V/A699V | + |
| 1411/1412 | L250I/G269R/L331S | + |
| 1413/1414 | G339E/G412A/E413K/A467N/I674V/A699V | + |
| 1415/1416 | G269R/G412A/E413K/I674V | + |
| 1417/1418 | R252L/L331S/G339E/G412A/E413K/A467N/A699V | + |
| 1419/1420 | L331S/A467N | + |
| 1421/1422 | E413K/I674V/A699V/R704K/P740H | + |
| 1423/1424 | R358N/I674V | + |
| 1425/1426 | K514G | + |
| 1427/1428 | W275F | + |
| 1429/1430 | G339E/R352K | + |
| 1431/1432 | R252L/G412A | + |
| 1433/1434 | R352K/A660P | + |
| 1435/1436 | V341A/A467N | + |
| 1437/1438 | G269R/E506N | + |
| 1439/1440 | R252L/G269R/R352K/A660P | + |
| 1441/1442 | P598L | + |
| 1443/1444 | L331S/A467N/E506N | + |
| 1445/1446 | R268G | + |
| 1447/1448 | R485M | + |
| 1449/1450 | G269R/G339E/E506N/R704K | + |
| 1451/1452 | P95G | + |
| 1453/1454 | E533P | + |
| 1455/1456 | W275L | + |
| 1457/1458 | V341A/A467N/E506N/A660P/I674V | + |
| 1459/1460 | L331S/R358N/A467N/I674V/A699V/R704K | + |
| 1461/1462 | R352K/E413K/A467N/E506N | + |
| 1463/1464 | R428Q | + |
| 1465/1466 | G339E/G412A/A467N/I674V | + |
| 1467/1468 | P323L | + |
| 1469/1470 | P95R | + |
| 1471/1472 | P122Q | + |
| 1473/1474 | G339E/A660P/I674V | + |
| 1475/1476 | S198H | + |
| 1477/1478 | A467N | + |
| 1479/1480 | R252L/G269R/G339E/R352K/G412A/A699V/R704K | + |
| 1481/1482 | L331S/E506N/I674V/A699V | + |
| 1483/1484 | R485T | + |
| 1485/1486 | K19A | + |
| 1487/1488 | R252L/G412A/E413K | + |
| 1489/1490 | G269R/R704K | + |
| 1491/1492 | R704K | + |
| 1493/1494 | G339E/E506N/R704K | + |
| 1495/1496 | R358N/E413K | + |
| 1497/1498 | E272S/R352K/E506N | + |
| 1499/1500 | R352G | + |
| 1501/1502 | R352K/R358N | + |
| 1503/1504 | R170L/A194V/A423Q/A451H/P557R/L658V/A660S/N743T | + |
| 1505/1506 | L301M | + |
| 1507/1508 | R170L/A451H/P557R | + |
| 1509/1510 | G339E/R352K/G412A/E419K/A467N/E506N/A660P/I674V/A699V | + |
| 1511/1512 | E209S | + |
| 1513/1514 | R252L/L331S/R358N/E506N | + |
| 1515/1516 | Q707S | + |
| 1517/1518 | E87R | + |
| 1519/1520 | L331S/A699V | + |
| 1521/1522 | L470G | + |
| 1523/1524 | R252L/L331S/G412A/E506N/R704K | + |
| 1525/1526 | P598H | + |
| 1527/1528 | R170L/A194V/A423Q/M476L/N743T | + |
| 1529/1530 | R252L/G339E/R352K/A467N/I674V/A699V | + |
| 1531/1532 | G269R/R358N | + |
| 1533/1534 | K517L | + |
| 1535/1536 | V341A/A467N/I674V | + |
| 1537/1538 | L470H | + |
| 1539/1540 | G269R/L599F/A660P/I674V/A699V | + |
| 1541/1542 | P159Q | + |
| 1543/1544 | L331S/G339E/G412A/E413K/A467N/E506N/I674V/R704K | + |

¹Levels of increased activity were determined relative to the reference polypeptide of SEQ ID NO: 178, and defined as follows: “+” > 0.85; “++” > 1.15; and “+++” > 2.

### Example 8

### Improvements Over SEQ ID NO: 178 in RT-qPCR Enzyme Activity

SEQ ID NO: 178 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR assay, as described below.

Each variant tested in a 15 μL reaction mixture comprising 1500 copies of SARS-CoV2 RNA fragment (Catalog #>CAT_10006625_2019-nCoV_N_Positive Control; IDT), containing the nucleocapsid gene, 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture from ThermoFisher), RT buffer (50 mM Tris-HCl, 73.1 mM KCl, 3.5 mM MgCl₂), 1.25 ng/uL of CODEX® HiTemp reverse transcriptase (Codexis), and 5 vol % HTP lysate of each variant was prepared. Then, RT-qPCR cycling (50° C. 15 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 8.1.

TABLE 8.1
Activity Relative to SEQ ID NO: 178

| SEQ ID NO: | Amino Acid Differences | FIOP Covid RT-qPCR |
|---|---|---|
| (nt/aa) | (Relative to SEQ ID NO: 178) | (Relative to SEQ ID NO: 178)¹ |
| 177/178 | | + |
| 1545/1546 | Q430A/L837S | ++ |
| 1425/1426 | K514G | ++ |
| 1547/1548 | E276G | ++ |
| 913/914 | K517T | ++ |
| 993/994 | N492Y | ++ |
| 845/846 | K517H | ++ |
| 1229/1230 | E17S | ++ |
| 991/992 | Y695K | ++ |
| 1549/1550 | E84T | ++ |
| 1029/1030 | Q689D | ++ |
| 1019/1020 | L15V | ++ |
| 1183/1184 | N228S | ++ |
| 1551/1552 | L301G | ++ |
| 1431/1432 | R252L/G412A | ++ |
| 1249/1250 | G269R/L331S/A699V | + |
| 1317/1318 | G269R/A467N/E506N | + |
| 1553/1554 | E696T | + |
| 1041/1042 | E433S | + |
| 1555/1556 | P598G | + |
| 1557/1558 | E276M | + |
| 951/952 | R596S | + |
| 1179/1180 | R268A/S621N | + |
| 731/732 | R170L/A194V/M476L/P557R/S659P | + |
| 1559/1560 | R252L | + |
| 1561/1562 | I674V | + |
| 1563/1564 | W275E | + |
| 789/790 | P557R/L658V/S659P | + |
| 967/968 | E783H | + |
| 1283/1284 | P14E | + |
| 795/796 | R170L/A423Q/M476L/L658V/H685A | + |
| 1173/1174 | K520V | + |
| 1511/1512 | E209S | + |
| 1565/1566 | L470M | + |
| 1567/1568 | E178G | + |
| 1215/1216 | R478L | + |
| 1347/1348 | E87A | + |
| 1159/1160 | Y695T | + |
| 741/742 | M476L/L658V/A660S/N743T | + |
| 1569/1570 | Y695S | + |
| 983/984 | K549V | + |
| 1239/1240 | R485G | + |
| 1083/1084 | G269R/G412A/A467N/R704K | + |
| 1571/1572 | L13G | + |
| 1289/1290 | E433K | + |
| 1007/1008 | N492R | + |
| 1573/1574 | R478M | + |
| 1031/1032 | E433V | + |
| 1319/1320 | R252L/A467N/E506N/I674V/R704K | + |
| 1243/1244 | E433R | + |
| 1575/1576 | M12V | + |
| 1477/1478 | A467N | + |
| 831/832 | M476L/P557R/L658V/S659P | + |
| 1291/1292 | R252L/G269R/I674V | + |
| 1577/1578 | L658V/S659P | + |
| 817/818 | E209W | + |
| 1027/1028 | R252L/R704K | + |
| 1227/1228 | R252L/G269R | + |
| 1579/1580 | E178L | + |
| 1581/1582 | E276A | + |
| 1095/1096 | R352K/I674V | + |
| 1087/1088 | L331S/R704K | + |
| 1221/1222 | K517R | + |
| 1583/1584 | Q430F | + |
| 955/956 | K517A | + |
| 1189/1190 | R252L/G412A/A467N/A660P/A699V/R704K | + |
| 1281/1282 | R252L/L331S/R352K/G412A/E506N/I674V | + |
| 1585/1586 | E87S | + |
| 1533/1534 | K517L | + |
| 1335/1336 | Q642E | + |
| 1379/1380 | R252L/R258H/L331S | + |
| 1587/1588 | R45S | + |
| 1589/1590 | W275M | + |
| 1305/1306 | P323Q | + |
| 1591/1592 | E276D | + |
| 1593/1594 | Q642R | + |
| 875/876 | P511L | + |
| 1043/1044 | P14V | + |
| 1063/1064 | R252L/L331S/A467N/A660P/I674V | + |
| 1595/1596 | N228D | + |
| 1407/1408 | R485I | + |
| 1597/1598 | E178N | + |
| 1353/1354 | P598R | + |
| 1599/1600 | E696G | + |
| 1601/1602 | E533L | + |
| 1603/1604 | E345P | + |
| 1187/1188 | Q707A | + |
| 1203/1204 | R478S | + |
| 1605/1606 | K249R | + |
| 1219/1220 | L331S/G412A/A660P/A699V | + |
| 1607/1608 | V534A | + |
| 1609/1610 | R478G | + |
| 1611/1612 | E17K | + |
| 1613/1614 | E264T | + |
| 1355/1356 | S198V | + |
| 1135/1136 | P598K | + |
| 873/874 | R170L/M476L/P557R/L658V/S659P | + |
| 1467/1468 | P323L | + |
| 999/1000 | G412A/I674V | + |
| 1615/1616 | P323S | + |
| 1093/1094 | L331S/R358N | + |
| 1617/1618 | W275G | + |
| 1169/1170 | R252L/E413K | + |
| 1471/1472 | P122Q | + |
| 1147/1148 | G269R/A699V/R704K | + |
| 1025/1026 | G412A/A467N/A660P/I674V/A699V | + |
| 1299/1300 | T725H | + |
| 1451/1452 | P95G | + |
| 1619/1620 | R477E | + |
| 1279/1280 | R252L/L331S/A699V | + |
| 1015/1016 | G412A/A660P | + |
| 1017/1018 | R252L/L331S | + |
| 1037/1038 | L331S/I674V | + |
| 1181/1182 | R252L/L331S/A660P | + |
| 1453/1454 | E533P | + |
| 1223/1224 | R252L/G269R/G412A/I674V | + |
| 1621/1622 | K19L/K214R | + |
| 1623/1624 | E345G | + |
| 1199/1200 | A699V/R704K | + |
| 1625/1626 | L470R | + |
| 1627/1628 | K565R | + |
| 1629/1630 | P598L | + |
| 747/748 | R170L/A423Q/P557R/L658V/N743T | + |
| 1213/1214 | E696K | + |
| 1631/1632 | E84N | + |
| 1633/1634 | W275V | + |
| 803/804 | M12E | + |
| 1635/1636 | G269C/Y695R | + |
| 745/746 | R170L/M476L/P557R/N743T | + |
| 771/772 | V534A/A670V | + |
| 859/860 | A194V/L658V/A660S | + |
| 1011/1012 | L222I | + |
| 1637/1638 | R485W | + |
| 1639/1640 | P511C | + |
| 1395/1396 | K214R | + |
| 1445/1446 | R268G | + |
| 1541/1542 | P159Q | + |
| 1057/1058 | P95D | + |
| 1271/1272 | G269R/I674V | + |
| 725/726 | R485A | + |
| 947/948 | K514V | + |
| 1641/1642 | E178S | + |
| 1643/1644 | E17G | + |
| 1483/1484 | R485T | + |
| 1645/1646 | E87R | + |
| 1185/1186 | R252L/R352K/G412A | + |

¹Levels of increased activity were determined relative to the reference polypeptide of SEQ ID NO: 178, and defined as follows: “+” >0.85; and “++” >1.15.

### Example 9

### Improvements Over SEQ ID NO: 178 in RT-qPCR Enzyme Activity

SEQ ID NO: 178 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR assay, as described below.

Each variant was tested in 15 μL reaction mixture comprising 5000 copies of non-infectious Influenza B (Inf-B) RNA fragment (Twist #>CAT_103003_Twist Synthetic Influenza B RNA Control, synthetic), and IDT 500 nM INFB primers (SEQ ID NOS: 3259 and 3260; CAT #10011562 and CAT #10011563), 125 nM INF-B Yikama Yellow (YAK) labeled probe (SEQ ID NO: 3261; IDT CAT #10011564), 0.2 mM dNTPs (10 mM mixture from ThermoFisher), RT buffer (50 mM Tris-HCl, 73.1 mM KCl, 3.5 mM MgCl₂), 1.25 ng/uL of CODEX® HiTemp reverse transcriptase (Codexis), and 20 vol % HTP lysate of each variant. Then, qPCR cycling (50C 15 min, 95° C. 2 min, 95° C. 5 sec, 60° C. 30 sec, for 50 cycles) was conducted in a CFX384 Touch Real-Time PCR Detection System (BioRad). For each variant tested, the Fold Improvement over Positive Control (FIOP) was calculated by the final relative fluorescence of the sample divided by the relative fluorescence of the control. The results are shown in Table 9.1.

TABLE 9.1
Activity Relative to SEQ ID NO: 178

| SEQ ID NO: | Amino Acid Differences | FIOP InfB RT-qPCR |
|---|---|---|
| (nt/aa) | (Relative to SEQ ID NO: 178) | (Relative to SEQ ID NO: 178)1 |
| 177/178 | | + |
| 1281/1282 | R252L/L331S/R352K/G412A/E506N/I674V | ++++ |
| 1545/1546 | Q430A/L837S | ++++ |
| 993/994 | N492Y | ++++ |
| 1169/1170 | R252L/E413K | ++++ |
| 1569/1570 | Y695S | ++++ |
| 1647/1648 | K517T | ++++ |
| 875/876 | P511L | ++++ |
| 1007/1008 | N492R | ++++ |
| 1173/1174 | K520V | ++++ |
| 1319/1320 | R252L/A467N/E506N/I674V/R704K | ++++ |
| 1083/1084 | G269R/G412A/A467N/R704K | ++++ |
| 1559/1560 | R252L | ++++ |
| 1425/1426 | K514G | +++ |
| 1611/1612 | E17K | +++ |
| 1271/1272 | G269R/I674V | +++ |
| 1623/1624 | E345G | +++ |
| 741/742 | M476L/L658V/A660S/N743T | +++ |
| 1017/1018 | R252L/L331S | +++ |
| 1087/1088 | L331S/R704K | +++ |
| 745/746 | R170L/M476L/P557R/N743T | +++ |
| 1159/1160 | Y695T | +++ |
| 1027/1028 | R252L/R704K | +++ |
| 1199/1200 | A699V/R704K | +++ |
| 1593/1594 | Q642R | +++ |
| 795/796 | R170L/A423Q/M476L/L658V/H685A | +++ |
| 947/948 | K514V | +++ |
| 1649/1650 | K249R | +++ |
| 1551/1552 | L301G | +++ |
| 1625/1626 | L470R | +++ |
| 1575/1576 | M12V | +++ |
| 1453/1454 | E533P | +++ |
| 1549/1550 | E84T | +++ |
| 1227/1228 | R252L/G269R | +++ |
| 1651/1652 | K355F | +++ |
| 1555/1556 | P598G | +++ |
| 1051/1052 | R485E | +++ |
| 1553/1554 | E696T | +++ |
| 1279/1280 | R252L/L331S/A699V | +++ |
| 1031/1032 | E433V | +++ |
| 845/846 | K517H | +++ |
| 995/996 | K565R | +++ |
| 1641/1642 | E178S | +++ |
| 1643/1644 | E17G | +++ |
| 1563/1564 | W275E | +++ |
| 861/862 | E264S | +++ |
| 1601/1602 | E533L | +++ |
| 955/956 | K517A | +++ |
| 1037/1038 | L331S/I674V | +++ |
| 1317/1318 | G269R/A467N/E506N | +++ |
| 999/1000 | G412A/I674V | +++ |
| 1613/1614 | E264T | +++ |
| 1595/1596 | N228D | +++ |
| 1019/1020 | L15V | +++ |
| 1183/1184 | N228S | +++ |
| 1639/1640 | P511C | +++ |
| 1653/1654 | I674V | +++ |
| 1573/1574 | R478M | +++ |
| 1511/1512 | E209S | +++ |
| 1095/1096 | R352K/I674V | +++ |
| 771/772 | V534A/A670V | +++ |
| 1655/1656 | P821R | +++ |
| 1547/1548 | E276G | +++ |
| 1607/1608 | V534A | ++ |
| 1041/1042 | E433S | ++ |
| 1093/1094 | L331S/R358N | ++ |
| 951/952 | R596S | ++ |
| 1633/1634 | W275V | ++ |
| 1239/1240 | R485G | ++ |
| 1657/1658 | E178G | ++ |
| 1631/1632 | E84N | ++ |
| 1557/1558 | E276M | ++ |
| 1571/1572 | L13G | ++ |
| 1249/1250 | G269R/L331S/A699V | ++ |
| 1579/1580 | E178L | ++ |
| 961/962 | L470E | ++ |
| 1347/1348 | E87A | ++ |
| 1431/1432 | R252L/G412A | ++ |
| 1029/1030 | Q689D | ++ |
| 1213/1214 | E696K | ++ |
| 1659/1660 | K249G | ++ |
| 1617/1618 | W275G | ++ |
| 1661/1662 | R322L | ++ |
| 1583/1584 | Q430F | ++ |
| 1355/1356 | S198V | ++ |
| 1467/1468 | P323L | ++ |
| 1663/1664 | E696W | ++ |
| 1591/1592 | E276D | ++ |
| 959/960 | K205R | ++ |
| 1597/1598 | E178N | ++ |
| 1011/1012 | L222I | ++ |
| 1243/1244 | E433R | ++ |
| 1665/1666 | P511E | ++ |
| 1477/1478 | A467N | ++ |
| 1063/1064 | R252L/L331S/A467N/A660P/I674V | ++ |
| 1043/1044 | P14V | ++ |
| 1219/1220 | L331S/G412A/A660P/A699V | ++ |
| 1609/1610 | R478G | ++ |
| 1581/1582 | E276A | ++ |
| 1541/1542 | P159Q | ++ |
| 1667/1668 | T725L | ++ |
| 1669/1670 | Q703K | ++ |
| 1291/1292 | R252L/G269R/I674V | ++ |
| 1585/1586 | E87S | ++ |
| 1637/1638 | R485W | ++ |
| 1671/1672 | E87Q | ++ |
| 1629/1630 | P598L | ++ |
| 721/722 | K565Y | ++ |
| 1645/1646 | E87R | ++ |
| 983/984 | K549V | ++ |
| 1565/1566 | L470M | ++ |
| 789/790 | P557R/L658V/S659P | ++ |
| 1177/1178 | R477E | ++ |
| 1229/1230 | E17S | ++ |
| 991/992 | Y695K | ++ |
| 1311/1312 | M12S | ++ |
| 1471/1472 | P122Q | ++ |
| 725/726 | R485A | ++ |
| 1599/1600 | E696G | + |
| 1223/1224 | R252L/G269R/G412A/I674V | + |
| 1189/1190 | R252L/G412A/A467N/A660P/A699V/R704K | + |
| 1407/1408 | R485I | + |

¹Levels of increased activity were determined relative to the reference polypeptide of SEQ ID NO: 178, and defined as follows: “+” >0.85; “++” >1.15; “+++” >3; and “++++” >8.

### Example 10

### Improvements Over SEQ ID NO: 178 in RT-qPCR Enzyme Activity and Inhibitor Tolerance

SEQ ID NO: 178 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR assay, as described below.

Each variant was tested in 15 μL reaction mixture comprising 250 copies of SARS-CoV2 RNA fragment (Catalog #>CAT_10006625_2019-nCoV_N_Positive Control; IDT) Twist CAT #102019, containing the nucleocapsid gene, 500 nM NI primers (SEQ ID NOS: 3262 and 3263), 125 nM FAM labeled probe (SEQ ID NO: 3264) (CDC EUA assay, Catalog #2019-nCoVEUA-01; IDT), 0.2 mM dNTPs (10 mM mixture from ThermoFisher), 2.5 CODEX® HiTemp reaction buffer, and 2.5 vol % HTP lysate of each variant tested. The reaction also comprised either a single inhibitor or a combination of two inhibitors selected from 3% ethanol, 25 mM GITC, 6.25 U/uL heparin and 10 μM hematin. RT-qPCR cycling (95° C. 2 min, 95° C. 3 sec, 55° C. 30 sec, for 45 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 10.1.

TABLE 10.1
Activity Relative to SEQ ID NO: 178

| SEQ ID NO: | Amino Acid Differences | FIOP Covid RT-qPCR with Inhibitor(s) |
|---|---|---|
| (nt/aa) | (Relative to SEQ ID NO: 178) | (Relative to SEQ ID NO: 178)¹ |
| 177/178 | | + |
| 875/876 | P511L | + |
| 993/994 | N492Y | + |
| 1569/1570 | Y695S | + |
| 1425/1426 | K514G | + |
| 1159/1160 | Y695T | + |
| 1545/1546 | Q430A/L837S | + |
| 1555/1556 | P598G | + |
| 1007/1008 | N492R | + |
| 1611/1612 | E17K | + |
| 1271/1272 | G269R/I674V | + |
| 1639/1640 | P511C | + |
| 1095/1096 | R352K/I674V | + |
| 1651/1652 | K355F | + |
| 1075/1076 | I674V | + |
| 947/948 | K514V | + |
| 1551/1552 | L301G | + |
| 1019/1020 | L15V | + |
| 1281/1282 | R252L/L331S/R352K/ | + |
| | G412A/E506N/I674V | |
| 1173/1174 | K520V | + |
| 1027/1028 | R252L/R704K | + |
| 1087/1088 | L331S/R704K | + |
| 1083/1084 | G269R/G412A/A467N/R704K | + |
| 1575/1576 | M12V | + |
| 1593/1594 | Q642R | + |
| 1669/1670 | Q703K | + |
| 1643/1644 | E17G | + |
| 1317/1318 | G269R/A467N/E506N | + |
| 1199/1200 | A699V/R704K | + |
| 1169/1170 | R252L/E413K | + |
| 1017/1018 | R252L/L331S | + |
| 1559/1560 | R252L | + |
| 1623/1624 | E345G | + |
| 1553/1554 | E696T | + |

¹Levels of increased activity were determined relative to the reference polypeptide of SEQ ID NO: 178, and defined as follows: “+” >0.85.

### Example 11

### Improvements Over SEQ ID NO: 994 in Enzyme Activity

SEQ ID NO: 994 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a qPCR assay as described below.

Each variant was tested in a 15 μL reaction mixture comprising 1500 copies of SARS-CoV2 DNA fragment (Catalog #>CAT_10006625_2019-nCoV_N_Positive Control; IDT), containing the nucleocapsid gene, 500 nM NI primers (SEQ ID NOS: 3262 and 3263), 125 nM FAM labeled probe (SEQ ID NO: 3264) (CDC EUA assay, Catalog #2019-nCoVEUA-01; IDT), 0.2 mM dNTPs (10 mM mixture from ThermoFisher), RT buffer (50 mM Tris-HCl, 73.1 mM KCl, 3.5 mM MgCl₂), and 2.5 vol % HTP lysate of each variant was prepared. Then, qPCR cycling (95° C. 2 min, 95° C. 3 sec, 55° C. 30 sec, for 45 cycles) was conducted in a CFX384 Touch Real-Time PCR Detection System (BioRad). For each variant tested, the Fold Improvement over Positive Control (FIOP) was calculated by the final relative fluorescence of the sample divided by the relative fluorescence of the control. The results are shown in Table 11.1.

TABLE 11.1
Activity Relative to SEQ ID NO: 994

| SEQ ID NO: | Amino Acid Differences | FIOP qPCR |
|---|---|---|
| (nt/aa) | (Relative to SEQ ID NO: 994) | (Relative to SEQ ID NO: 994)¹ |
| 993/994 | | + |
| 1673/1674 | A194V/R358N/R704K | ++++ |
| 1675/1676 | A194V/A467N/L658V | ++++ |
| 1677/1678 | A194V/R252L/R358N/G412A/A467N/L658V/ | ++++ |
| | E696T/N743T | |
| 1679/1680 | A194V/G412A | ++++ |
| 1681/1682 | A194V/A467N/N743T | ++++ |
| 1683/1684 | R252L | ++++ |
| 1685/1686 | A194V/R252L | ++++ |
| 1687/1688 | A194V/R252L/N743T | ++++ |
| 1689/1690 | A194V/A467N/P511C/L658V | ++++ |
| 1691/1692 | L666T | ++++ |
| 1693/1694 | L38R | +++ |
| 1695/1696 | L38G | +++ |
| 1697/1698 | T518R | +++ |
| 1699/1700 | P311C | +++ |
| 1701/1702 | P300R | +++ |
| 1703/1704 | R151K | +++ |
| 1705/1706 | H5G | +++ |
| 1707/1708 | E440N | +++ |
| 1709/1710 | A194V/R358N/L658V | +++ |
| 1711/1712 | P300N | +++ |
| 1713/1714 | A194V/R252L/R358N/G412A/A467N | +++ |
| 1715/1716 | L658A | +++ |
| 1717/1718 | A194V/N743T | +++ |
| 1719/1720 | L666G | +++ |
| 1721/1722 | A194V/R252L/R358N | +++ |
| 1723/1724 | A194V/A467N/R704K | +++ |
| 1725/1726 | A194V | +++ |
| 1727/1728 | A194V/R252L/G412A/E696T | +++ |
| 1729/1730 | D375I | +++ |
| 1731/1732 | A194V/R358N | +++ |
| 1733/1734 | A194V/G412A/A467N | +++ |
| 1735/1736 | L658V/R704K | +++ |
| 1737/1738 | A194V/R252L/A467N/E696T/N743T | +++ |
| 1739/1740 | E807H | +++ |
| 1741/1742 | R252L/A467N/E696T | +++ |
| 1743/1744 | R252L/G412A/L658V | ++ |
| 1745/1746 | A194V/R358N/G412A | ++ |
| 1747/1748 | R358N | ++ |
| 1749/1750 | K718R | ++ |
| 1751/1752 | E218T | ++ |
| 1753/1754 | A194V/G412A/A467N/N743T | ++ |
| 1755/1756 | Y32L | ++ |
| 1757/1758 | M12V/R252L/E413K/Y492R | ++ |
| 1759/1760 | R704Q | ++ |
| 1761/1762 | R804Q | ++ |
| 1763/1764 | E529T | ++ |
| 1765/1766 | G40N | ++ |
| 1767/1768 | E529R | ++ |
| 1769/1770 | P300W | ++ |
| 1771/1772 | E304T | ++ |
| 1773/1774 | K645L | ++ |
| 1775/1776 | A560K | ++ |
| 1777/1778 | L666D | ++ |
| 1779/1780 | P740G | ++ |
| 1781/1782 | E481D | ++ |
| 1783/1784 | L666V | ++ |
| 1785/1786 | R804D | ++ |
| 1787/1788 | F81A | ++ |
| 1789/1790 | P300A | ++ |
| 1791/1792 | E304C | ++ |
| 1793/1794 | V746R | ++ |
| 1795/1796 | L658C | ++ |
| 1797/1798 | V746G | ++ |
| 1799/1800 | E814R | ++ |
| 1801/1802 | L331D | ++ |
| 1803/1804 | P694S | ++ |
| 1805/1806 | L666M | ++ |
| 1807/1808 | R704V | ++ |
| 1809/1810 | L666K | ++ |
| 1811/1812 | L38V | ++ |
| 1813/1814 | L38E | ++ |
| 1815/1816 | P694T | ++ |
| 1817/1818 | F35L/E304G | ++ |
| 1819/1820 | L666S | ++ |
| 1821/1822 | L658R | ++ |
| 1823/1824 | S289G | ++ |
| 1825/1826 | L666R | ++ |
| 1827/1828 | E829R | ++ |
| 1829/1830 | R258L/P347H | ++ |
| 1831/1832 | H5V | ++ |
| 1833/1834 | E218Y | ++ |
| 1835/1836 | E829L | ++ |
| 1837/1838 | K279R | ++ |
| 1839/1840 | R340T | + |
| 1841/1842 | L242N | + |
| 1843/1844 | S490V | + |
| 1845/1846 | R284A | + |
| 1847/1848 | D375A/L376I | + |
| 1849/1850 | E814A | + |
| 1851/1852 | E329L | + |
| 1853/1854 | R639Q | + |
| 1855/1856 | E721W | + |
| 1857/1858 | M12V/L15V/G269R/K355F/G412A/E413K/ | + |
| | Y492R/R704K | |
| 1859/1860 | V746Q | + |
| 1861/1862 | E529V | + |
| 1863/1864 | S80G | + |
| 1865/1866 | R420T | + |
| 1867/1868 | H7Q | + |
| 1869/1870 | R420S | + |
| 1871/1872 | K645R | + |
| 1873/1874 | H2G | + |
| 1875/1876 | E635I | + |
| 1877/1878 | P300T | + |
| 1879/1880 | G40S | + |
| 1881/1882 | H5W | + |
| 1883/1884 | N592S | + |
| 1885/1886 | E814I | + |
| 1887/1888 | K718S | + |
| 1889/1890 | E717T | + |
| 1891/1892 | E304G | + |
| 1893/1894 | S289D | + |
| 1895/1896 | R782S | + |
| 1897/1898 | E440T | + |
| 1899/1900 | G8R | + |
| 1901/1902 | N592A | + |
| 1903/1904 | K713V | + |
| 1905/1906 | P300L | + |
| 1907/1908 | E406M | + |
| 1909/1910 | G40R | + |
| 1911/1912 | L242G | + |
| 1913/1914 | A560G | + |
| 1915/1916 | E298A | + |
| 1917/1918 | H489R | + |
| 1919/1920 | L331Y | + |
| 1921/1922 | L226E | + |
| 1923/1924 | R33K | + |
| 1925/1926 | H5A | + |
| 1927/1928 | H2R | + |
| 1929/1930 | R434K | + |
| 1931/1932 | L658E | + |
| 1933/1934 | E481G | + |
| 1935/1936 | T518L | + |
| 1937/1938 | H169S | + |
| 1939/1940 | L331Q | + |
| 1941/1942 | E65S | + |
| 1943/1944 | E298S | + |
| 1945/1946 | K645M | + |
| 1947/1948 | P300F | + |
| 1949/1950 | L666N | + |
| 1951/1952 | S490R | + |
| 1953/1954 | L38S | + |
| 1955/1956 | E140M | + |
| 1957/1958 | E329S | + |
| 1959/1960 | E721V | + |
| 1961/1962 | E304V | + |
| 1963/1964 | H685T | + |
| 1965/1966 | Q543A | + |

¹Levels of increased activity were determined relative to the reference polypeptide of SEQ ID NO: 994, and defined as follows: “+” >0.85; “++” >1.15; “+++” >3; and “++++” >10.

### Example 12

### Improvements Over SEQ ID NO: 994 in Enzyme Activity

SEQ ID NO: 994 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR assay, as described below.

For each variant tested, a 15 μL reaction mixture comprising 1500 copies of SARS-CoV2 RNA fragment (Catalog #>CAT_10006625_2019-nCoV_N_Positive Control; IDT), containing the nucleocapsid gene, 500 nM NI primers (SEQ ID NOS: 3262 and 3563), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture from ThermoFisher), RT buffer (50 mM Tris-HCl, 73.1 mM KCl, 3.5 mM MgCl₂), 1.25 ng/uL of CODEX® HiTemp reverse transcriptase (Codexis), and 5 vol % HTP lysate of each variant was prepared. Then, RT-qPCR cycling (50C 15 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 12.1.

TABLE 12.1
Activity Relative to SEQ ID NO: 994

| SEQ ID | Amino Acid Differences | FIOP Covid RT-qPCR |
|---|---|---|
| NO: | (Relative to | (Relative to |
| (nt/aa) | SEQ ID NO: 994) | SEQ ID NO: 994)¹ |
| 993/994 | | + |
| 1967/1968 | Q543A | +++ |
| 1969/1970 | H5L | +++ |
| 1857/1858 | M12V/L15V/G269R/K355F/ | +++ |
| | G412A/E413K/Y492R/R704K | |
| 1971/1972 | L331G | +++ |
| 1973/1974 | R782A | +++ |
| 1975/1976 | D230G | +++ |
| 1977/1978 | E721R | +++ |
| 1855/1856 | E721W | +++ |
| 1979/1980 | A560G | +++ |
| 1919/1920 | L331Y | ++ |
| 1879/1880 | G40S | ++ |
| 1917/1918 | H489R | ++ |
| 1981/1982 | H2V | ++ |
| 1983/1984 | E814M | ++ |
| 1911/1912 | L242G | ++ |
| 1889/1890 | E717T | ++ |
| 1799/1800 | E814R | ++ |
| 1985/1986 | E406G | ++ |
| 1953/1954 | L38S | ++ |
| 1961/1962 | E304V | ++ |
| 1757/1758 | M12V/R252L/E413K/Y492R | ++ |
| 1987/1988 | R284G | ++ |
| 1989/1990 | P311G | ++ |
| 1991/1992 | E807G | ++ |
| 1993/1994 | L242A | ++ |
| 1995/1996 | E65H | ++ |
| 1997/1998 | P309G | ++ |
| 1929/1930 | R434K | ++ |
| 1891/1892 | E304G | ++ |
| 1807/1808 | R704V | ++ |
| 1999/2000 | E65G | ++ |
| 2001/2002 | E304R | ++ |
| 1693/1694 | L38R | ++ |
| 1943/1944 | E298S | ++ |
| 2003/2004 | L242R | ++ |
| 1865/1866 | R420T | ++ |
| 1825/1826 | L666R | ++ |
| 2005/2006 | G40R | ++ |
| 2007/2008 | Q543R | ++ |
| 1821/1822 | L658R | ++ |
| 1893/1894 | S289D | ++ |
| 1829/1830 | R258L/P347H | ++ |
| 2009/2010 | P740L | ++ |
| 1951/1952 | S490R | ++ |
| 2011/2012 | L658Q | ++ |
| 1915/1916 | E298A | + |
| 2013/2014 | H685F | + |
| 1955/1956 | E140M | + |
| 2015/2016 | E441R | + |
| 1843/1844 | S490V | + |
| 1901/1902 | N592A | + |
| 2017/2018 | P300R | + |
| 2019/2020 | D375E | + |
| 1837/1838 | K279R | + |
| 2021/2022 | E721D | + |
| 1835/1836 | E829L | + |
| 1861/1862 | E529V | + |
| 2023/2024 | E721M | + |
| 1711/1712 | P300N | + |
| 1851/1852 | E329L | + |
| 1903/1904 | K713V | + |
| 2025/2026 | L38G | + |
| 2027/2028 | T518M | + |
| 2029/2030 | Q543L | + |
| 1859/1860 | V746Q | + |
| 1863/1864 | S80G | + |
| 1883/1884 | N592S | + |
| 1873/1874 | H2G | + |
| 2031/2032 | E829R | + |
| 1811/1812 | L38V | + |
| 1831/1832 | H5V | + |
| 1783/1784 | L666V | + |
| 1947/1948 | P300F | + |
| 1869/1870 | R420S | + |
| 2033/2034 | L666G | + |
| 1875/1876 | E635I | + |
| 2035/2036 | R284A | + |
| 2037/2038 | S80M | + |
| 1871/1872 | K645R | + |
| 2039/2040 | P740S | + |
| 1703/1704 | R151K | + |
| 1739/1740 | E807H | + |
| 1921/1922 | L226E | + |
| 1791/1792 | E304C | + |
| 1899/1900 | G8R | + |
| 1705/1706 | H5G | + |
| 2041/2042 | N743S | + |
| 1699/1700 | P311C | + |
| 1925/1926 | H5A | + |
| 1801/1802 | L331D | + |
| 2043/2044 | K718R | + |
| 1885/1886 | E814I | + |
| 1833/1834 | E218Y | + |
| 2045/2046 | L666Q | + |
| 2047/2048 | R804G | + |
| 1839/1840 | R340T | + |
| 2049/2050 | D375L | + |
| 1853/1854 | R639Q | + |
| 1697/1698 | T518R | + |
| 1941/1942 | E65S | + |
| 1847/1848 | D375A/L376I | + |
| 1881/1882 | H5W | + |

¹Levels of increased activity were determined relative to the reference polypeptide of SEQ ID NO: 994, and defined as follows: “+” >0.85; “++” >1.15; and “+++” >1.5.

### Example 13

### Improvements Over SEQ ID NO: 994 in Enzyme Activity

SEQ ID NO: 994 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR assay, as described below.

Each variant was tested in a 15 μL reaction mixture comprising 5000 copies of non-infectious Influenza B (Inf-B) RNA fragment (Twist #>CAT_103003_Twist Synthetic Influenza B RNA Control, synthetic), and 500 nM INFB primers (SEQ ID NOS: 3259 and 3260; CAT #10011562 and CAT #10011563; IDT), 125 nM INF-B YAK labeled probe (SEQ ID NO: 3261; CAT #10011564; IDT), 0.2 mM dNTPs (10 mM mixture from ThermoFisher), RT buffer (50 mM Tris-HCl, 73.1 mM KCl, 3.5 mM MgCl₂), 1.25 ng/uL of CODEX® HiTemp reverse transcriptase, and 1.167 vol % HTP lysate of each variant was prepared. Then, qPCR cycling (50° C. 15 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 13.1.

TABLE 13.1
Activity Relative to SEQ ID NO: 994

| SEQ ID | Amino Acid Differences | FIOP InfB RT-qPCR |
|---|---|---|
| NO: (nt/aa) | (Relative to SEQ ID NO: 994) | (Relative to SEQ ID NO: 994)¹ |
| 993/994 | | + |
| 1977/1978 | E721R | +++ |
| 1917/1918 | H489R | +++ |
| 1973/1974 | R782A | +++ |
| 1857/1858 | M12V/L15V/G269R/K355F/G412A/E413K/Y492R/R704K | +++ |
| 1951/1952 | S490R | +++ |
| 1961/1962 | E304V | +++ |
| 1971/1972 | L331G | +++ |
| 1901/1902 | N592A | +++ |
| 2051/2052 | E98G | +++ |
| 1757/1758 | M12V/R252L/E413K/Y492R | +++ |
| 1889/1890 | E717T | +++ |
| 2003/2004 | L242R | +++ |
| 2011/2012 | L658Q | ++ |
| 1993/1994 | L242A | ++ |
| 1865/1866 | R420T | ++ |
| 1855/1856 | E721W | ++ |
| 1949/1950 | L666N | ++ |
| 2023/2024 | E721M | ++ |
| 1991/1992 | E807G | ++ |
| 1891/1892 | E304G | ++ |
| 1959/1960 | E721V | ++ |
| 2053/2054 | E440S | ++ |
| 2049/2050 | D375L | ++ |
| 2027/2028 | T518M | ++ |
| 1995/1996 | E65H | ++ |
| 1983/1984 | E814M | ++ |
| 1989/1990 | P311G | ++ |
| 1969/1970 | H5L | ++ |
| 1793/1794 | V746R | ++ |
| 2007/2008 | Q543R | ++ |
| 2055/2056 | H2P | ++ |
| 1897/1898 | E440T | ++ |
| 1931/1932 | L658E | ++ |
| 1825/1826 | L666R | ++ |
| 1875/1876 | E635I | ++ |
| 1981/1982 | H2V | ++ |
| 1919/1920 | L331Y | ++ |
| 1947/1948 | P300F | ++ |
| 1911/1912 | L242G | ++ |
| 1821/1822 | L658R | ++ |
| 2057/2058 | K713S | ++ |
| 1929/1930 | R434K | ++ |
| 1895/1896 | R782S | ++ |
| 2013/2014 | H685F | ++ |
| 1879/1880 | G40S | ++ |
| 2021/2022 | E721D | ++ |
| 2059/2060 | E721L | ++ |
| 1809/1810 | L666K | ++ |
| 1859/1860 | V746Q | ++ |
| 1829/1830 | R258L/P347H | ++ |
| 2061/2062 | E441G | ++ |
| 1799/1800 | E814R | ++ |
| 1765/1766 | G40N | ++ |
| 1903/1904 | K713V | ++ |
| 2063/2064 | K227Y | ++ |
| 1791/1792 | E304C | ++ |
| 1955/1956 | E140M | ++ |
| 1937/1938 | H169S | ++ |
| 1883/1884 | N592S | ++ |
| 1873/1874 | H2G | ++ |
| 1843/1844 | S490V | ++ |
| 2039/2040 | P740S | ++ |
| 1871/1872 | K645R | ++ |
| 1943/1944 | E298S | ++ |
| 1913/1914 | A560G | ++ |
| 2065/2066 | E98N | ++ |
| 1899/1900 | G8R | ++ |
| 1835/1836 | E829L | ++ |
| 1703/1704 | R151K | ++ |
| 2067/2068 | D375E | ++ |
| 2069/2070 | R284G | ++ |
| 1869/1870 | R420S | ++ |
| 1939/1940 | L331Q | ++ |
| 2071/2072 | E635T | ++ |
| 1887/1888 | K718S | ++ |
| 1965/1966 | Q543A | ++ |
| 2029/2030 | Q543L | ++ |
| 1921/1922 | L226E | ++ |
| 2073/2074 | D375R | ++ |
| 2075/2076 | E218G | ++ |
| 2077/2078 | L658S | ++ |
| 2079/2080 | E65G | ++ |
| 2081/2082 | G692S | ++ |
| 2083/2084 | E717Y | ++ |
| 2085/2086 | E635G | ++ |
| 1933/1934 | E481G | ++ |
| 1907/1908 | E406M | ++ |
| 1781/1782 | E481D | ++ |
| 1839/1840 | R340T | ++ |
| 1837/1838 | K279R | ++ |
| 1853/1854 | R639Q | ++ |
| 1997/1998 | P309G | + |
| 1957/1958 | E329S | + |
| 1763/1764 | E529T | + |
| 1833/1834 | E218Y | + |
| 2087/2088 | K645N | + |
| 1941/1942 | E65S | + |
| 2089/2090 | D375M | + |
| 1783/1784 | L666V | + |
| 1775/1776 | A560K | + |
| 2091/2092 | R340S | + |
| 1811/1812 | L38V | + |
| 1719/1720 | L666G | + |
| 1759/1760 | R704Q | + |
| 2009/2010 | P740L | + |
| 2093/2094 | E406Q | + |
| 1831/1832 | H5V | + |
| 2095/2096 | L666E | + |
| 1847/1848 | D375A/L376I | + |
| 1851/1852 | E329L | + |
| 2045/2046 | L666Q | + |
| 1877/1878 | P300T | + |
| 1827/1828 | E829R | + |
| 2097/2098 | D375A | + |
| 1861/1862 | E529V | + |
| 1945/1946 | K645M | + |
| 1807/1808 | R704V | + |
| 1785/1786 | R804D | + |
| 1849/1850 | E814A | + |
| 1773/1774 | K645L | + |
| 1915/1916 | E298A | + |
| 1935/1936 | T518L | + |
| 2099/2100 | E529L | + |
| 1803/1804 | P694S | + |
| 2101/2102 | K645F | + |
| 1777/1778 | L666D | + |
| 1755/1756 | Y32L | + |
| 2103/2104 | D230G | + |
| 1789/1790 | P300A | + |
| 2105/2106 | L331V | + |
| 1797/1798 | V746G | + |
| 2107/2108 | R804A | + |
| 2109/2110 | P202Q | + |
| 1819/1820 | L666S | + |

¹Levels of increased activity were determined relative to the reference polypeptide of SEQ ID NO: 994, and defined as follows: “+” > 0.85; “++” > 1.15; and “+++” > 1.5.

### Example 14

### Improvements Over SEQ ID NO: 994 in Enzyme Activity and Inhibitor Tolerance

SEQ ID NO: 994 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR assay in the presence of inhibitors, as described below.

For each variant tested, a 15 μL reaction mixture comprising 1250 copies of non-infectious Influenza B (Inf-B) RNA fragment (Twist #>CAT_103003_Twist Synthetic Influenza B RNA Control, synthetic), and 500 nM INFB primers (SEQ ID NOS: 3262 and 3263; CAT #10011562 and CAT #10011563, IDT), 125 nM INFB (YAK) probe (SEQ ID NO: 3261; CAT #10011564, IDT), 0.2 mM dNTPs (10 mM mixture from ThermoFisher), 2.5× CODEX® HiTemp reaction buffer (Codexis) diluted to 1×, 1.25 ng/uL of CODEX® HiTemp reverse transcriptase, and 5 vol % HTP lysate of each variant was prepared. Then, qPCR cycling (50C 15 min, 95C 2 min, 95C 5 sec, 60C 30 sec, for 50 cycles) was conducted in a CFX384 Touch Real-Time PCR Detection System (BioRad). The reaction mixtures also comprised either a single inhibitor or a combination of two inhibitors selected from 1.5% ethanol, 25 mM GITC, 10 μM hematin, 6.25 U/mL heparin, 0.01% SDS, 150 mM urea, 37.5 mM NaCl and 250 ng gDNA (human genomic DNA; Promega). For each variant, the Fold Improvement over Positive Control (FIOP) was calculated by the final relative fluorescence of the sample divided by the relative fluorescence of the control. The results are shown in Table 14.1.

TABLE 14.1
Activity Relative to SEQ ID NO: 994

| SEQ ID NO: | Amino Acid Differences | FIOP InfB RT-qPCR with Inhibitor(s) |
|---|---|---|
| (nt/aa) | (Relative to SEQ ID NO: 994) | (Relative to SEQ ID NO: 994)¹ |
| 993/994 | | + |
| 1757/1758 | M12V/R252L/E413K/Y492R | ++++ |
| 1901/1902 | N592A | ++++ |
| 1857/1858 | M12V/L15V/G269R/K355F/G412A/E413K/Y492R/R704K | ++++ |
| 1961/1962 | E304V | ++++ |
| 1929/1930 | R434K | ++++ |
| 1979/1980 | A560G | ++++ |
| 1977/1978 | E721R | ++++ |
| 1971/1972 | L331G | ++++ |
| 1891/1892 | E304G | ++++ |
| 2013/2014 | H685F | ++++ |
| 2111/2112 | A194V/R358N/A467N/L658V/E696T/R704K | ++++ |
| 1737/1738 | A194V/R252L/A467N/E696T/N743T | ++++ |
| 1677/1678 | A194V/R252L/R358N/G412A/A467N | ++++ |
| | /L658V/E696T/N743T | |
| 1727/1728 | A194V/R252L/G412A/E696T | ++++ |
| 2113/2114 | A194V/R252L/E696T/R704K | ++++ |
| 1741/1742 | R252L/A467N/E696T | +++ |
| 1931/1932 | L658E | +++ |
| 2115/2116 | A194V/A467N/L658V/N743T | +++ |
| 1829/1830 | R258L/P347H | +++ |
| 1825/1826 | L666R | +++ |
| 1733/1734 | A194V/G412A/A467N | +++ |
| 1967/1968 | Q543A | +++ |
| 1713/1714 | A194V/R252L/R358N/G412A/A467N | +++ |
| 2117/2118 | R252L/R358N/E696T/R704K | +++ |
| 2119/2120 | A194V/L658V | +++ |
| 1675/1676 | A194V/A467N/L658V | +++ |
| 1889/1890 | E717T | +++ |
| 1717/1718 | A194V/N743T | +++ |
| 2121/2122 | A194V/R252L/R704K/N743T | ++ |
| 2123/2124 | A194V/R252L/A467N/L658V | ++ |
| 2125/2126 | A194V/R252L/A467N/R704K | ++ |
| 1917/1918 | H489R | ++ |
| 1723/1724 | A194V/A467N/R704K | ++ |
| 1725/1726 | A194V | ++ |
| 2023/2024 | E721M | ++ |
| 1681/1682 | A194V/A467N/N743T | ++ |
| 1709/1710 | A194V/R358N/L658V | ++ |
| 2127/2128 | A194V/G412A/L658V | ++ |
| 1685/1686 | A194V/R252L | ++ |
| 1911/1912 | L242G | ++ |
| 1679/1680 | A194V/G412A | ++ |
| 2035/2036 | R284A | ++ |
| 1687/1688 | A194V/R252L/N743T | ++ |
| 1745/1746 | A194V/R358N/G412A | + |
| 1983/1984 | E814M | + |
| 1743/1744 | R252L/G412A/L658V | + |
| 1731/1732 | A194V/R358N | + |
| 2129/2130 | A194V/A467N | + |

¹Levels of increased activity were determined relative to the reference polypeptide of SEQ ID NO: 994, and defined as follows: “+” > 0.85; “++” > 1.15; “+++” > 3; and “++++” > 10.

### Example 15

### Improvements Over SEQ ID NO: 1858 in Enzyme Activity

SEQ ID NO: 1858 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR assay as described below.

For each variant tested, a 15 μL reaction mixture comprising 375 copies of SARS-CoV2 RNA fragment (Catalog #>CAT_10006625_2019-nCoV_N_Positive Control; IDT), containing the nucleocapsid gene, 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture from ThermoFisher), RT buffer (50 mM Tris-HCl, 73.1 mM KCl, 3.5 mM MgCl₂), 1.25 ng/uL of CODEX® HiTemp reverse transcriptase (Codexis), and 5 vol % HTP lysate of each variant was prepared. Then, RT-qPCR cycling (50° C. 15 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 15.1.

TABLE 15.1
Activity Relative to SEQ ID NO: 1858

| SEQ ID NO: | Amino Acid Differences | FIOP Covid RT-qPCR |
|---|---|---|
| (nt/aa) | (Relative to SEQ ID NO: 1858) | (Relative to SEQ ID NO: 1858)¹ |
| 1857/1858 | | + |
| 2131/2132 | R269G/L658V | ++ |
| 2133/2134 | R358N/L658V | ++ |
| 2135/2136 | A194V/A467N/E721R | ++ |
| 2137/2138 | E304G/H489R/L658V/E721R | ++ |
| 2139/2140 | H489R | ++ |
| 2141/2142 | R252L/R269G/E304V/N743T | ++ |
| 2143/2144 | R252L/E304G/L658V | + |
| 2145/2146 | E304V | + |
| 2147/2148 | R269G/E304G/L658E | + |
| 2149/2150 | A194V/R252L/E304G/A467N | + |
| 2151/2152 | R269G | + |
| 2153/2154 | A194V/R252L/A467N | + |
| 2155/2156 | A194V/P536G/L658E | + |
| 2157/2158 | E717T | + |
| 2159/2160 | A194V/R269G/E304V/L658V/E717T | + |
| 2161/2162 | A194V/R269G | + |
| 2163/2164 | E304G | + |
| 2165/2166 | A194V/R252L/R358N/H489R | + |
| 2167/2168 | E721R | + |
| 2169/2170 | E304V/H489R/L658E | + |
| 2171/2172 | R252L/R358N/A467N/H489R/H685F | + |
| 2173/2174 | A194V/E304V/R358N/A467N/H489R | + |
| 2175/2176 | H685F | + |
| 2177/2178 | A194V/R252L/R269G/E304G/A467N | + |
| 2179/2180 | R269G/E721R | + |
| 2181/2182 | R269G/E304V/A467N | + |
| 2183/2184 | E304G/H685F | + |
| 2185/2186 | R252L/E304G/H489R/E696T/E717T | + |
| 2187/2188 | H489R/E696T/N743T | + |
| 2189/2190 | A194V/R269G/E717T | + |
| 2191/2192 | R269G/E304G | + |
| 2193/2194 | R252L | + |
| 2195/2196 | R252L/H685F | + |
| 2197/2198 | E304G/A467N/H489R/E717T | + |
| 2199/2200 | E304G/R358N/A467N/N592A | + |
| 2201/2202 | A194V/R269G/E304G | + |
| 2203/2204 | A194V/R252L/E304G/R358N/A467N/L658E | + |
| 2205/2206 | A194V/R252L/E304G | + |
| 2207/2208 | A194V/E721R | + |
| 2209/2210 | E696T | + |

¹Levels of increased activity were determined relative to the reference polypeptide of SEQ ID NO: 1858 and defined as follows: “+” > 0.85; and “++” > 1.15.

### Example 16

### Improvements Over SEQ ID NO: 1858 in Enzyme Activity

SEQ ID NO: 1858 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR reaction, as described below.

Each variant was tested in 15 μL reaction mixture comprising 625 copies of non-infectious Influenza B (Inf-B) RNA fragment (Twist #>CAT_103003_Twist Synthetic Influenza B RNA Control, synthetic), and 500 nM INFB primers (SEQ ID NOS: 3259 and 3260; CAT #10011562 and CAT #10011563, IDT), 125 nM INF-B YAK labeled probe (SEQ ID NO: 3261; CAT #10011564, IDT); 2.5× CODEX® HiTemp reaction buffer (Codexis) diluted to 1×, 1.25 ng/uL of CODEX® HiTemp reverse transcriptase (Codexis), and 1.33 vol % HTP lysate of each variant was prepared. Then, qPCR cycling (50° C. 15 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 16.1.

TABLE 16.1
Activity Relative to SEQ ID NO: 1858

| SEQ ID NO: | Amino Acid Differences | FIOP InfB RT-qPCR |
|---|---|---|
| (nt/aa) | (Relative to SEQ ID NO: 1858) | (Relative to SEQ ID NO: 1858)¹ |
| 1857/1858 | | + |
| 2167/2168 | E721R | ++ |
| 2197/2198 | E304G/A467N/H489R/E717T | ++ |
| 2139/2140 | H489R | ++ |
| 2209/2210 | E696T | ++ |
| 2133/2134 | R358N/L658V | + |
| 2175/2176 | H685F | + |
| 2185/2186 | R252L/E304G/H489R/E696T/E717T | + |
| 2211/2212 | R269G/E304G/H489R/H685F | + |
| 2137/2138 | E304G/H489R/L658V/E721R | + |
| 2131/2132 | R269G/L658V | + |
| 2157/2158 | E717T | + |
| 2213/2214 | E304V | + |
| 2171/2172 | R252L/R358N/A467N/H489R/H685F | + |
| 2179/2180 | R269G/E721R | + |
| 2169/2170 | E304V/H489R/L658E | + |
| 2215/2216 | R269G | + |
| 2181/2182 | R269G/E304V/A467N | + |
| 2195/2196 | R252L/H685F | + |
| 2183/2184 | E304G/H685F | + |
| 2193/2194 | R252L | + |
| 2217/2218 | R269G/A467N/H489R/L658V/H685F | + |
| 2143/2144 | R252L/E304G/L658V | + |
| 2219/2220 | R252L/E304V | + |
| 2187/2188 | H489R/E696T/N743T | + |

¹Levels of increased activity were determined relative to the reference polypeptide of SEQ ID NO: 1858, and defined as follows: “+” > 0.85; and “++” > 1.15.

### Example 17

### Improvements Over SEQ ID NO: 1858 in Enzyme Activity and Inhibitor Tolerance

SEQ ID NO: 1858 was selected as the parent enzyme for the round of evolution and enzyme characterization described in this Example. The polypeptides encoded by each gene were produced in HTP as described in Example 2, and the soluble lysate was generated as described in Example 3. Each variant was screened in a RT-qPCR assay, as described below.

Each variant was tested in a 15 μL reaction mixture comprising 625 copies of non-infectious Influenza B (Inf-B) RNA fragment (Twist #>CAT_103003_Twist Synthetic Influenza B RNA Control, synthetic), and 500 nM INFB primers (SEQ ID NOS: 3259 and 3260; CAT #10011562 and CAT #10011563; IDT), 125 nM INF-B (YAK) labeled probe (SEQ ID NO: 3261; IDT CAT #10011564), 2.5× CODEX® HiTemp reaction buffer (Codexis) diluted to 1×, 1.25 ng/uL of CODEX® HiTemp reverse transcriptase (Codexis), and 1.33 vol % HTP lysate of each variant were prepared. Then, qPCR cycling (50° C. 15 min, 95° C. 2 min, 95° C. 5 sec, 60° C. 30 sec, for 50 cycles) was conducted in a CFX384 Touch Real-Time PCR Detection System (BioRad). The reaction also comprised either a single inhibitor or a combination of two inhibitors selected from 3% ethanol, 25 mM GITC, 2.5 μM hematin, 1.6 U/mL heparin, 0.005% SDS, 300 mM urea, 37.5 mM NaCl, and 16.7 ng/uL gDNA. For each variant, the Fold Improvement over Positive Control (FIOP) was calculated by the final relative fluorescence of the sample divided by the relative fluorescence of the control.

The results are shown in Table 17.1.

TABLE 17.1
Activity Relative to SEQ ID NO: 1858

| SEQ ID | Amino Acid Differences | FIOP InfB RT-qPCR with Inhibitor(s) |
|---|---|---|
| NO: (nt/aa) | (Relative to SEQ ID NO: 1858) | (Relative to SEQ ID NO: 1858)¹ |
| 1857/1858 | | + |
| 2137/2138 | E304G/H489R/L658V/E721R | +++ |
| 2175/2176 | H685F | +++ |
| 2195/2196 | R252L/H685F | +++ |
| 2209/2210 | E696T | +++ |
| 2167/2168 | E721R | +++ |
| 2139/2140 | H489R | +++ |
| 2183/2184 | E304G/H685F | +++ |
| 2197/2198 | E304G/A467N/H489R/E717T | +++ |
| 2157/2158 | E717T | ++ |
| 2221/2222 | R269G | ++ |
| 2131/2132 | R269G/L658V | ++ |
| 2133/2134 | R358N/L658V | + |

¹Levels of increased activity were determined relative to the reference polypeptide of SEQ ID NO: 1858, and defined as follows: “+” > 0.85; “++” > 1.15; and “+++” > 1.5.

### Example 18

### Improvements Over SEQ ID NO: 2176 in Enzyme Activity

SEQ ID NO: 2176 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR reaction, as describe below.

Each variant was tested in 15 μL reaction mixture comprising 375 copies of SARS-CoV2 RNA fragment (Catalog #>CAT_10006625_2019-nCoV_N_Positive Control; IDT), containing the nucleocapsid gene, 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture from ThermoFisher), RT buffer (50 mM Tris-HCl, 73.1 mM KCl, 3.5 mM MgCl₂), 1.25 ng/uL of CODEX® HiTemp reverse transcriptase (Codexis), and 5 vol % HTP lysate of each variant was prepared. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 18.1.

TABLE 18.1
Activity Relative to SEQ ID NO: 2176

| SEQ ID | Amino Acid Differences | FIOP Covid RT-qPCR |
|---|---|---|
| NO: (nt/aa) | (Relative to SEQ ID NO: 2176) | (Relative to SEQ ID NO: 2176)¹ |
| 2175/2176 | | + |
| 2223/2224 | K227E/E443D/M756R | +++ |
| 2225/2226 | E65N/K233S/Q543V/S621R | +++ |
| 2227/2228 | Q543V/A699S/E814T | +++ |
| 2229/2230 | R45S/A699Q/S748G/V749K | +++ |
| 2231/2232 | R45L/S621R | +++ |
| 2233/2234 | R358N/A467N | +++ |
| 2235/2236 | R268G/G431T/R605K/V749K | +++ |
| 2237/2238 | T553V | +++ |
| 2239/2240 | L331S/R605K | +++ |
| 2241/2242 | T597N | +++ |
| 2243/2244 | R45S/A243N/E312Q/R340T/A423E/Q543A/E814T | +++ |
| 2245/2246 | R45S/E65N/A243N/R268Q | +++ |
| 2247/2248 | R252L | +++ |
| 2249/2250 | R45S/R340T/A423E/E814T | +++ |
| 2251/2252 | R45S/H489R/P557R/V749K | +++ |
| 2253/2254 | H489R/P510R | +++ |
| 2255/2256 | R340T/H489R | +++ |
| 2257/2258 | H489R/S621R/E814T | +++ |
| 2259/2260 | E304G/H489R | +++ |
| 2261/2262 | R268Q/A423E/G431T/Q543A/P557R/R605K/E814T | +++ |
| 2263/2264 | G519L | +++ |
| 2265/2266 | E533T | +++ |
| 2267/2268 | H489R | ++ |
| 2269/2270 | S621K | ++ |
| 2271/2272 | E696Q | ++ |
| 2273/2274 | R45S/R268G/E312Q/R605K/S748G/V749K/E814T | ++ |
| 2275/2276 | H292E/Q430H | ++ |
| 2277/2278 | P510R | ++ |
| 2279/2280 | R45S/R340T/P557R | ++ |
| 2281/2282 | R45S/R268Q/H489R/P557R/S748G/V749K | ++ |
| 2283/2284 | S621R | ++ |
| 2285/2286 | R45S/A243N/E312Q/L331A/P557R | ++ |
| 2287/2288 | P302V/N494V/A525R | ++ |
| 2289/2290 | R340T/H489R/A699Q | ++ |
| 2291/2292 | R45S/G431T | ++ |
| 2293/2294 | E312Q/R340T/N494V | ++ |
| 2295/2296 | V538M | ++ |
| 2297/2298 | R45S/S221D/A243N/R340T/A423E | ++ |
| 2299/2300 | R268Q/E312Q/L331A/A423E/A451G/P557R/R605K | ++ |
| 2301/2302 | K227E/E443D/N592S/A617V | ++ |
| 2303/2304 | E329T | ++ |
| 2305/2306 | E65S | ++ |
| 2307/2308 | R268Q/A423E/E814T | ++ |
| 2309/2310 | R45S/E475H/P557R/S748G/V749K | ++ |
| 2311/2312 | P510D | ++ |
| 2313/2314 | E406W | ++ |
| 2315/2316 | S748G/V749K | ++ |
| 2317/2318 | H292R | ++ |
| 2319/2320 | R268Q/E312Q/L331A/R340T/A699Q | ++ |
| 2321/2322 | A530V | ++ |
| 2323/2324 | R45S/A423E | ++ |
| 2325/2326 | R45S/A451G | ++ |
| 2327/2328 | T42V | ++ |
| 2329/2330 | P302V | ++ |
| 2331/2332 | R268Q/R340T/R352Q/A423E/A451G | ++ |
| 2333/2334 | K233R | ++ |
| 2335/2336 | F355T | ++ |
| 2337/2338 | E109H/R340T/H489R/P510R/S659G | ++ |
| 2339/2340 | A423R | ++ |
| 2341/2342 | A660V | ++ |
| 2343/2344 | R268Q | ++ |
| 2345/2346 | R45S/E312Q/R340T/R605K | ++ |
| 2347/2348 | P557R | ++ |
| 2349/2350 | K210R | ++ |
| 2351/2352 | N494Y | ++ |
| 2353/2354 | E814Q | ++ |
| 2355/2356 | E329S | ++ |
| 2357/2358 | A194V/R358N | ++ |
| 2359/2360 | G519F | ++ |
| 2361/2362 | E721R | ++ |
| 2363/2364 | A660G | ++ |
| 2365/2366 | P300T | ++ |
| 2367/2368 | K227E | ++ |
| 2369/2370 | P302V/N494V/Q543V/S659G/E814T | ++ |
| 2371/2372 | R352Q | ++ |
| 2373/2374 | S659C | ++ |
| 2375/2376 | M756L | ++ |
| 2377/2378 | E298G | ++ |
| 2379/2380 | P557S | ++ |
| 2381/2382 | L301G/V538M/E546A/S621R/E814T | ++ |
| 2383/2384 | K64E/E293H | ++ |
| 2385/2386 | E397V | ++ |
| 2387/2388 | R269L | ++ |
| 2389/2390 | P510R/S748G | ++ |
| 2391/2392 | R492A | ++ |
| 2393/2394 | R45S/A243N/E312Q/L331A/A451G | ++ |
| 2395/2396 | S221C | ++ |
| 2397/2398 | R268G/A423E/E814T | ++ |
| 2399/2400 | G519V | ++ |
| 2401/2402 | G431T | ++ |
| 2403/2404 | E312Q | ++ |
| 2405/2406 | H292G | ++ |
| 2407/2408 | R284E | ++ |
| 2409/2410 | F685V | ++ |
| 2411/2412 | R45L | ++ |
| 2413/2414 | G488S | ++ |
| 2415/2416 | F685G | ++ |
| 2417/2418 | A451G | ++ |
| 2419/2420 | R269I | ++ |
| 2421/2422 | A530G | ++ |
| 2423/2424 | W407V | ++ |
| 2425/2426 | V534G | ++ |
| 2427/2428 | T597G | ++ |
| 2429/2430 | K57R | ++ |
| 2431/2432 | H489R/N494V | ++ |
| 2433/2434 | F355V | ++ |
| 2435/2436 | V534A | ++ |
| 2437/2438 | P557M | ++ |
| 2439/2440 | P302V/R340T/N494V | ++ |
| 2441/2442 | R340K | ++ |
| 2443/2444 | A94K | ++ |
| 2445/2446 | K210S | ++ |
| 2447/2448 | R269S | ++ |
| 2449/2450 | E406L | ++ |
| 2451/2452 | G488A | ++ |
| 2453/2454 | R268G | ++ |
| 2455/2456 | N494V | ++ |
| 2457/2458 | E109C | ++ |
| 2459/2460 | P300L | + |
| 2461/2462 | S621Q | + |
| 2463/2464 | P557L | + |
| 2465/2466 | E834L | + |
| 2467/2468 | L301C | + |
| 2469/2470 | L331V | + |
| 2471/2472 | A463W | + |
| 2473/2474 | R45V | + |
| 2475/2476 | L331K | + |
| 2477/2478 | Q543A | + |
| 2479/2480 | A560S | + |
| 2481/2482 | P536D | + |
| 2483/2484 | E283S | + |
| 2485/2486 | A280K | + |
| 2487/2488 | P694T | + |
| 2489/2490 | E814M | + |
| 2491/2492 | V749S | + |
| 2493/2494 | E717D | + |
| 2495/2496 | R268Q/A423E | + |
| 2497/2498 | A560M | + |
| 2499/2500 | E65N | + |
| 2501/2502 | L666S | + |
| 2503/2504 | R269G/L658E | + |
| 2505/2506 | W407L | + |
| 2507/2508 | E814K | + |
| 2509/2510 | L331M | + |
| 2511/2512 | R268V | + |
| 2513/2514 | Y738P | + |
| 2515/2516 | M756I | + |
| 2517/2518 | P302L/R340T/H489R/N494S/P510R/S748G/E814T | + |
| 2519/2520 | L242Q | + |
| 2521/2522 | L301E | + |
| 2523/2524 | A350S | + |
| 2525/2526 | E329M | + |
| 2527/2528 | A699S | + |
| 2529/2530 | P557R/R605K/A699Q/E721R | + |
| 2531/2532 | E65C | + |
| 2533/2534 | L331A | + |
| 2535/2536 | A94M | + |
| 2537/2538 | R45S/E109H/R340T/R605K/E814T | + |
| 2539/2540 | R269V | + |
| 2541/2542 | E721A | + |
| 2543/2544 | R686G | + |
| 2545/2546 | R151S | + |
| 2547/2548 | E475C | + |
| 2549/2550 | E466M | + |
| 2551/2552 | L331S | + |
| 2553/2554 | A699Q | + |
| 2555/2556 | R352Q/A451G/H489R/Q543V/P557R/E721R | + |
| 2557/2558 | A530W | + |
| 2559/2560 | R118T | + |
| 2561/2562 | R340T | + |
| 2563/2564 | R340V | + |
| 2565/2566 | E533P | + |
| 2567/2568 | R268E | + |
| 2569/2570 | P536G | + |
| 2571/2572 | E722V | + |
| 2573/2574 | R284G | + |
| 2575/2576 | R45M | + |
| 2577/2578 | N494R | + |
| 2579/2580 | V749T | + |
| 2581/2582 | H489R/L658E | + |
| 2583/2584 | R340L | + |
| 2585/2586 | K61G | + |
| 2587/2588 | A243N | + |
| 2589/2590 | V749G | + |
| 2591/2592 | S659A | + |
| 2593/2594 | E109H | + |
| 2595/2596 | E481S | + |
| 2597/2598 | Q145F | + |
| 2599/2600 | G692R | + |
| 2601/2602 | A530D | + |
| 2603/2604 | H292M | + |
| 2605/2606 | E312D | + |
| 2607/2608 | E329V | + |
| 2609/2610 | S621G | + |
| 2611/2612 | R340A | + |
| 2613/2614 | P510L | + |
| 2615/2616 | S621D | + |
| 2617/2618 | S659T | + |
| 2619/2620 | G40C | + |
| 2621/2622 | A350G | + |
| 2623/2624 | A530T | + |
| 2625/2626 | T553Q | + |
| 2627/2628 | S221T | + |
| 2629/2630 | E65K/H489R/Q543A | + |
| 2631/2632 | R269G | + |
| 2633/2634 | F355C | + |
| 2635/2636 | V749K | + |
| 2637/2638 | A94R | + |
| 2639/2640 | S221D | + |
| 2641/2642 | K802R | + |
| 2643/2644 | F124A | + |
| 2645/2646 | R639K | + |
| 2647/2648 | E109T | + |
| 2649/2650 | T211L | + |
| 2651/2652 | R521W | + |
| 2653/2654 | T597V | + |
| 2655/2656 | E814R | + |
| 2657/2658 | R804A | + |
| 2659/2660 | G40S | + |
| 2661/2662 | S621T | + |
| 2663/2664 | K233S | + |
| 2665/2666 | R102D | + |
| 2667/2668 | A194G | + |
| 2669/2670 | S748G | + |
| 2671/2672 | R102E | + |
| 2673/2674 | K210G | + |
| 2675/2676 | H292E | + |
| 2677/2678 | L316T | + |
| 2679/2680 | L666W | + |
| 2681/2682 | R102T | + |
| 2683/2684 | A423E | + |
| 2685/2686 | R420H | + |
| 2687/2688 | E466K | + |
| 2689/2690 | E272V | + |
| 2691/2692 | P300G | + |
| 2693/2694 | A280G | + |
| 2695/2696 | R45S/A243N/E814T | + |
| 2697/2698 | E829R | + |
| 2699/2700 | A699G | + |
| 2701/2702 | G488R | + |
| 2703/2704 | K802C | + |
| 2705/2706 | E441L | + |
| 2707/2708 | R605K | + |
| 2709/2710 | F355L/Q430R | + |
| 2711/2712 | R118L | + |
| 2713/2714 | T597S | + |
| 2715/2716 | A530E | + |
| 2717/2718 | A280E | + |
| 2719/2720 | R371S | + |
| 2721/2722 | K233L | + |
| 2723/2724 | R118V | + |
| 2725/2726 | W177F | + |
| 2727/2728 | E312Q/L331A/R605K/E814T | + |
| 2729/2730 | G644R | + |
| 2731/2732 | L242V | + |
| 2733/2734 | N494H | + |
| 2735/2736 | K704R | + |
| 2737/2738 | R45S | + |
| 2739/2740 | Q543L | + |
| 2741/2742 | R501V | + |
| 2743/2744 | R268K | + |
| 2745/2746 | E814G | + |
| 2747/2748 | E827R | + |
| 2749/2750 | A243N/R268G | + |
| 2751/2752 | P300S | + |
| 2753/2754 | P300M | + |
| 2755/2756 | V534W | + |
| 2757/2758 | T211R | + |
| 2759/2760 | E441M | + |
| 2761/2762 | R102G | + |
| 2763/2764 | A463L | + |
| 2765/2766 | N743G | + |
| 2767/2768 | G431V | + |
| 2769/2770 | E475H | + |
| 2771/2772 | R45G | + |
| 2773/2774 | F81W | + |
| 2775/2776 | P536T | + |
| 2777/2778 | A243S | + |
| 2779/2780 | E722W | + |
| 2781/2782 | A350C | + |
| 2783/2784 | E272L | + |
| 2785/2786 | R269M | + |
| 2787/2788 | L732M | + |
| 2789/2790 | H292W | + |
| 2791/2792 | P302V/N494V | + |
| 2793/2794 | E406G | + |
| 2795/2796 | Q186M | + |
| 2797/2798 | E109Y | + |
| 2799/2800 | P510M | + |
| 2801/2802 | A670M | + |
| 2803/2804 | P300E | + |
| 2805/2806 | R804G | + |
| 2807/2808 | K233E | + |
| 2809/2810 | R340T/H489R/N494V/Q543A/S659G/E814T | + |
| 2811/2812 | E481A | + |
| 2813/2814 | S289E | + |
| 2815/2816 | V534R | + |
| 2817/2818 | A94L | + |
| 2819/2820 | K57I | + |
| 2821/2822 | S621E | + |
| 2823/2824 | E298I | + |
| 2825/2826 | E466H | + |
| 2827/2828 | L301G | + |
| 2829/2830 | R371C | + |
| 2831/2832 | P323C | + |
| 2833/2834 | R726M | + |
| 2835/2836 | A560V | + |
| 2837/2838 | A194V/R269G/H489R | + |
| 2839/2840 | E223D | + |

¹Levels of increased activity were determined relative to the reference polypeptide of SEQ ID NO: 2176, and defined as follows: “+” > 0.85; “++” > 1.15; and “+++” > 1.5.

### Example 19

Improvements over SEQ ID NO: 2176 in Enzyme Activity

SEQ ID NO: 2176 was selected as the parent enzyme the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR reaction as described below.

Each variant was tested in a 15 μL reaction mixture comprising 375 copies of non-infectious Influenza B (Inf-B) RNA fragment (Twist #>CAT_103003_Twist Synthetic Influenza B RNA Control, synthetic), and 500 nM INFB primers (SEQ ID NOS: 3259 and 3260; CAT #10011562 and CAT #10011563; IDT), 125 nM INF-B (YAK) labeled probe (SEQ ID NO: 3261; IDT CAT #10011564), 2.5× CODEX® HiTemp reaction buffer (Codexis) diluted to 1×, 1.25 ng/uL of CODEX® HiTemp reverse transcriptase (Codexis), and 2.5 vol % HTP lysate of each variant. Then, qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 19.1.

TABLE 19.1
Activity Relative to SEQ ID NO: 2176

| | | FIOP InfB |
|---|---|---|
| | | RT-qPCR |
| | | (Relative |
| SEQ ID | Amino Acid Differences | to SEQ ID |
| NO: (nt/aa) | (Relative to SEQ ID NO: 2176) | NO: 2176)¹ |
| 2175/2176 | | + |
| 2273/2274 | R45S/R268G/E312Q/R605K/S748G/V749K/E814T | +++ |
| 2841/2842 | R45S/E109H/A243N/A423E/Q543V/R605K/A699Q/S748G/V749K/E814T | +++ |
| 2253/2254 | H489R/P510R | +++ |
| 2309/2310 | R45S/E475H/P557R/S748G/V749K | +++ |
| 2281/2282 | R45S/R268Q/H489R/P557R/S748G/V749K | +++ |
| 2843/2844 | R45S/R268Q/H489R/Q543V/P557R/S659T/E721R | +++ |
| 2297/2298 | R45S/S221D/A243N/R340T/A423E | +++ |
| 2229/2230 | R45S/A699Q/S748G/V749K | +++ |
| 2251/2252 | R45S/H489R/P557R/V749K | +++ |
| 2517/2518 | P302L/R340T/H489R/N494S/P510R/S748G/E814T | +++ |
| 2845/2846 | E696Q | +++ |
| 2601/2602 | A530D | +++ |
| 2315/2316 | S748G/V749K | +++ |
| 2389/2390 | P510R/S748G | +++ |
| 2319/2320 | R268Q/E312Q/L331A/R340T/A699Q | +++ |
| 2749/2750 | A243N/R268G | +++ |
| 2393/2394 | R45S/A243N/E312Q/L331A/A451G | +++ |
| 2287/2288 | P302V/N494V/A525R | +++ |
| 2285/2286 | R45S/A243N/E312Q/L331A/P557R | +++ |
| 2847/2848 | P302L/R340T/H489R/N494V/A525R | +++ |
| 2849/2850 | E312Q/N494V/A525R/Q543V/S659G/S748G/E814T | +++ |
| 2261/2262 | R268Q/A423E/G431T/Q543A/P557R/R605K/E814T | +++ |
| 2427/2428 | T597G | +++ |
| 2495/2496 | R268Q/A423E | +++ |
| 2529/2530 | P557R/R605K/A699Q/E721R | +++ |
| 2299/2300 | R268Q/E312Q/L331A/A423E/A451G/P557R/R605K | +++ |
| 2263/2264 | G519L | ++ |
| 2851/2852 | E312Q/A423E/G431T/A451G/H489R | ++ |
| 2629/2630 | E65K/H489R/Q543A | ++ |
| 2853/2854 | A423E/A463W/R605K | ++ |
| 2855/2856 | R45S/A243N/E312Q/R605K | ++ |
| 2799/2800 | P510M | ++ |
| 2241/2242 | T597N | ++ |
| 2857/2858 | E109H/R340T/H489R/P510R/S659G | ++ |
| 2791/2792 | P302V/N494V | ++ |
| 2859/2860 | R45S/R340T/E475H/P557R/E814T | ++ |
| 2397/2398 | R268G/A423E/E814T | ++ |
| 2361/2362 | E721R | ++ |
| 2861/2862 | V538M | ++ |
| 2277/2278 | P510R | ++ |
| 2291/2292 | R45S/G431T | ++ |
| 2555/2556 | R352Q/A451G/H489R/Q543V/P557R/E721R | ++ |
| 2863/2864 | Q543V | ++ |
| 2499/2500 | E65N | ++ |
| 2243/2244 | R45S/A243N/E312Q/R340T/A423E/Q543A/E814T | ++ |
| 2245/2246 | R45S/E65N/A243N/R268Q | ++ |
| 2431/2432 | H489R/N494V | ++ |
| 2693/2694 | A280G | ++ |
| 2233/2234 | R358N/A467N | ++ |
| 2727/2728 | E312Q/L331A/R605K/E814T | ++ |
| 2641/2642 | K802R | ++ |
| 2865/2866 | H489R | ++ |
| 2553/2554 | A699Q | ++ |
| 2381/2382 | L301G/V538M/E546A/S621R/E814T | ++ |
| 2345/2346 | R45S/E312Q/R340T/R605K | ++ |
| 2867/2868 | S621R | ++ |
| 2869/2870 | H292R | ++ |
| 2527/2528 | A699S | ++ |
| 2359/2360 | G519F | ++ |
| 2871/2872 | R252L | ++ |
| 2873/2874 | R102P | ++ |
| 2557/2558 | A530W | ++ |
| 2875/2876 | P557R | ++ |
| 2411/2412 | R45L | ++ |
| 2593/2594 | E109H | ++ |
| 2877/2878 | V749G | ++ |
| 2567/2568 | R268E | ++ |
| 2331/2332 | R268Q/R340T/R352Q/A423E/A451G | ++ |
| 2587/2588 | A243N | ++ |
| 2325/2326 | R45S/A451G | ++ |
| 2479/2480 | A560S | ++ |
| 2351/2352 | N494Y | ++ |
| 2879/2880 | S221D | ++ |
| 2265/2266 | E533T | ++ |
| 2343/2344 | R268Q | ++ |
| 2695/2696 | R45S/A243N/E814T | ++ |
| 2581/2582 | H489R/L658E | ++ |
| 2419/2420 | R269I | ++ |
| 2501/2502 | L666S | ++ |
| 2289/2290 | R340T/H489R/A699Q | ++ |
| 2307/2308 | R268Q/A423E/E814T | ++ |
| 2881/2882 | P300L | ++ |
| 2453/2454 | R268G | ++ |
| 2793/2794 | E406G | ++ |
| 2883/2884 | R45S/E312Q | ++ |
| 2417/2418 | A451G | ++ |
| 2623/2624 | A530T | ++ |
| 2571/2572 | E722V | ++ |
| 2679/2680 | L666W | ++ |
| 2531/2532 | E65C | ++ |
| 2425/2426 | V534G | ++ |
| 2885/2886 | E475C | ++ |
| 2827/2828 | L301G | ++ |
| 2887/2888 | E304G/H489R | ++ |
| 2467/2468 | L301C | ++ |
| 2889/2890 | P323W | ++ |
| 2809/2810 | R340T/H489R/N494V/Q543A/S659G/E814T | ++ |
| 2533/2534 | L331A | ++ |
| 2691/2692 | P300G | ++ |
| 2715/2716 | A530E | ++ |
| 2371/2372 | R352Q | ++ |
| 2583/2584 | R340L | ++ |
| 2891/2892 | V749S | ++ |
| 2635/2636 | V749K | + |
| 2893/2894 | S162T | + |
| 2621/2622 | A350G | + |
| 2439/2440 | P302V/R340T/N494V | + |
| 2227/2228 | Q543V/A699S/E814T | + |
| 2895/2896 | H489R/N494V/S659G | + |
| 2897/2898 | K64H/E293D | + |
| 2541/2542 | E721A | + |
| 2899/2900 | R269G | + |
| 2269/2270 | S621K | + |
| 2435/2436 | V534A | + |
| 2561/2562 | R340T | + |
| 2901/2902 | R269G/L658E | + |
| 2507/2508 | E814K | + |
| 2449/2450 | E406L | + |
| 2739/2740 | Q543L | + |
| 2483/2484 | E283S | + |
| 2401/2402 | G431T | + |
| 2469/2470 | L331V | + |
| 2773/2774 | F81W | + |
| 2903/2904 | E283L | + |
| 2757/2758 | T211R | + |
| 2905/2906 | E283A | + |
| 2235/2236 | R268G/G431T/R605K/V749K | + |
| 2239/2240 | L331S/R605K | + |
| 2471/2472 | A463W | + |
| 2617/2618 | S659T | + |
| 2907/2908 | E109H/A423E | + |
| 2525/2526 | E329M | + |
| 2335/2336 | F355T | + |
| 2365/2366 | P300T | + |
| 2909/2910 | A467N | + |
| 2305/2306 | E65S | + |
| 2911/2912 | E329S | + |
| 2487/2488 | P694T | + |
| 2751/2752 | P300S | + |
| 2667/2668 | A194G | + |
| 2595/2596 | E481S | + |
| 2443/2444 | A94K | + |
| 2913/2914 | L666E | + |
| 2781/2782 | A350C | + |
| 2375/2376 | M756L | + |
| 2723/2724 | R118V | + |
| 2915/2916 | A423E | + |
| 2423/2424 | W407V | + |
| 2709/2710 | F355L/Q430R | + |
| 2323/2324 | R45S/A423E | + |
| 2769/2770 | E475H | + |
| 2771/2772 | R45G | + |
| 2521/2522 | L301E | + |
| 2403/2404 | E312Q | + |
| 2917/2918 | E120D | + |
| 2821/2822 | S621E | + |
| 2741/2742 | R501V | + |
| 2919/2920 | R258L | + |
| 2249/2250 | R45S/R340T/A423E/E814T | + |
| 2707/2708 | R605K | + |
| 2737/2738 | R45S | + |
| 2713/2714 | T597S | + |
| 2473/2474 | R45V | + |
| 2921/2922 | P300E | + |
| 2689/2690 | E272V | + |
| 2537/2538 | R45S/E109H/R340T/R605K/E814T | + |
| 2255/2256 | R340T/H489R | + |
| 2777/2778 | A243S | + |
| 2923/2924 | M476V | + |
| 2711/2712 | R118L | + |
| 2925/2926 | E475L | + |
| 2753/2754 | P300M | + |
| 2927/2928 | E298D | + |
| 2333/2334 | K233R | + |
| 2825/2826 | E466H | + |
| 2699/2700 | A699G | + |
| 2279/2280 | R45S/R340T/P557R | + |
| 2231/2232 | R45L/S621R | + |
| 2929/2930 | R151G | + |
| 2549/2550 | E466M | + |
| 2931/2932 | R284G | + |
| 2767/2768 | G431V | + |
| 2703/2704 | K802C | + |
| 2633/2634 | F355C | + |
| 2933/2934 | A350Y | + |
| 2935/2936 | L245T | + |
| 2823/2824 | E298I | + |
| 2669/2670 | S748G | + |
| 2937/2938 | E814T | + |

¹Levels of increased activity were determined relative to the reference polypeptide of SEQ ID NO: 2176, and defined as follows: “+” > 0.85; “++” > 1.15; and “+++” > 1.5.

### Example 20

### Improvements Over SEQ ID NO: 2176 in Enzyme Activity and Inhibitor Tolerance

SEQ ID NO: 2176 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR reaction, as described below.

Each variant was tested in a 15 μL reaction mixture comprising 375 copies of non-infectious Influenza B (Inf-B) RNA fragment (Twist #>CAT_103003_Twist Synthetic Influenza B RNA Control, synthetic), and 500 nM INFB primers (SEQ ID NOS: 3259 and 3260; CAT #10011562 and CAT #10011563; IDT), 125 nM INFB (YAK) labeled probe (SEQ ID NO: 3261; CAT #10011564, IDT), 2.5× CODEX® HiTemp reaction buffer (Codexis) diluted to 1×, 1.25 ng/uL of CODEX® HiTemp reverse transcriptase (Codexis), and 2.5 vol % HTP lysate of each variant was prepared. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). The reaction also comprised either a single inhibitor or a combination of two inhibitors selected from 3% ethanol, 25 mM GITC, 2 μM hematin, 1.25 U/mL heparin, 0.005% SDS, 240 mM urea, 37.5 mM NaCl, and 250 ng gDNA. For each variant, the Fold Improvement over Positive Control (FIOP) was calculated by the final relative fluorescence of the sample divided by the relative fluorescence of the control. The results are shown in Table 20.1.

TABLE 20.1 Activity Relative to SEQ ID NO: 2176

| | | FIOP InfB RT-qPCR with |
|---|---|---|
| SEQ ID | Amino Acid Differences | Inhibitor(s) |
| NO: (nt/aa) | (Relative to SEQ ID NO: 2176) | (Relative to SEQ ID NO: 2176)¹ |
| 2175/2176 | | + |
| 2863/2864 | Q543V | ++++ |
| 2669/2670 | S748G | +++ |
| 2629/2630 | E65K/H489R/Q543A | +++ |
| 2617/2618 | S659T | +++ |
| 2635/2636 | V749K | +++ |
| 2561/2562 | R340T | +++ |
| 2703/2704 | K802C | +++ |
| 2223/2224 | K227E/E443D/M756R | +++ |
| 2273/2274 | R45S/R268G/E312Q/R605K/S748G/V749K/E814T | +++ |
| 2849/2850 | E312Q/N494V/A525R/Q543V/S659G/S748G/E814T | +++ |
| 2281/2282 | R45S/R268Q/H489R/P557R/S748G/V749K | +++ |
| 2715/2716 | A530E | +++ |
| 2623/2624 | A530T | +++ |
| 2403/2404 | E312Q | +++ |
| 2309/2310 | R45S/E475H/P557R/S748G/V749K | +++ |
| 2443/2444 | A94K | +++ |
| 2937/2938 | E814T | +++ |
| 2601/2602 | A530D | +++ |
| 2517/2518 | P302L/R340T/H489R/N494S/P510R/S748G/E814T | +++ |
| 2229/2230 | R45S/A699Q/S748G/V749K | +++ |
| 2251/2252 | R45S/H489R/P557R/V749K | +++ |
| 2287/2288 | P302V/N494V/A525R | +++ |
| 2939/2940 | E696Q | +++ |
| 2679/2680 | L666W | ++ |
| 2593/2594 | E109H | ++ |
| 2361/2362 | E721R | ++ |
| 2499/2500 | E65N | ++ |
| 2773/2774 | F81W | ++ |
| 2529/2530 | P557R/R605K/A699Q/E721R | ++ |
| 2365/2366 | P300T | ++ |
| 2381/2382 | L301G/V538M/E546A/S621R/E814T | ++ |
| 2595/2596 | E481S | ++ |
| 2299/2300 | R268Q/E312Q/L331A/A423E/A451G/P557R/R605K | ++ |
| 2847/2848 | P302L/R340T/H489R/N494V/A525R | ++ |
| 2253/2254 | H489R/P510R | ++ |
| 2501/2502 | L666S | ++ |
| 2261/2262 | R268Q/A423E/G431T/Q543A/P557R/R605K/E814T | ++ |
| 2389/2390 | P510R/S748G | ++ |
| 2941/2942 | R268Q | ++ |
| 2913/2914 | L666E | ++ |
| 2297/2298 | R45S/S221D/A243N/R340T/A423E | ++ |
| 2769/2770 | E475H | ++ |
| 2315/2316 | S748G/V749K | ++ |
| 2227/2228 | Q543V/A699S/E814T | ++ |
| 2453/2454 | R268G | ++ |
| 2943/2944 | P510R | ++ |
| 2245/2246 | R45S/E65N/A243N/R268Q | ++ |
| 2945/2946 | R358N/A467N | ++ |
| 2289/2290 | R340T/H489R/A699Q | ++ |
| 2879/2880 | S221D | ++ |
| 2581/2582 | H489R/L658E | ++ |
| 2903/2904 | E283L | ++ |
| 2875/2876 | P557R | ++ |
| 2865/2866 | H489R | ++ |
| 2319/2320 | R268Q/E312Q/L331A/R340T/A699Q | ++ |
| 2707/2708 | R605K | ++ |
| 2947/2948 | P300M | ++ |
| 2371/2372 | R352Q | ++ |
| 2553/2554 | A699Q | ++ |
| 2401/2402 | G431T | ++ |
| 2857/2858 | E109H/R340T/H489R/P510R/S659G | ++ |
| 2871/2872 | R252L | + |
| 2887/2888 | E304G/H489R | + |
| 2899/2900 | R269G | + |
| 2827/2828 | L301G | + |
| 2325/2326 | R45S/A451G | + |
| 2549/2550 | E466M | + |
| 2471/2472 | A463W | + |
| 2533/2534 | L331A | + |
| 2547/2548 | E475C | + |
| 2417/2418 | A451G | + |
| 2911/2912 | E329S | + |
| 2231/2232 | R45L/S621R | + |
| 2571/2572 | E722V | + |
| 2737/2738 | R45S | + |
| 2873/2874 | R102P | + |
| 2751/2752 | P300S | + |
| 2449/2450 | E406L | + |
| 2479/2480 | A560S | + |

¹Levels of increased activity were determined relative to the reference polypeptide of SEQ ID NO: 2176, and defined as follows: “+” > 0.85; “++” > 1.15; “+++” > 3; and “++++” > 10.

### Example 21

### Improvements Over SEQ ID NO: 2176 in Enzyme Activity and Inhibitor Tolerance

SEQ ID NO: 2176 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened a RT-qPCR reaction, as described below.

Each variant was tested in a 15 μL reaction mixture comprising 75 copies of SARS-CoV2 RNA fragment (Catalog #>CAT_10006625_2019-nCoV_N_Positive Control; IDT) Twist CAT #102019, containing the nucleocapsid gene, 500 nM N1 primers (SEQ ID NOS: 3262 and 3263), 125 nM FAM labeled probe (SEQ ID NO: 3264) (CDC EUA assay, 2019-nCoVEUA-01; IDT), 2.5× CODEX® HiTemp reaction buffer (Codexis) diluted to 1×, and 2.5 vol % HTP lysate of each variant was prepared. The reaction also comprised either a single inhibitor or a combination of two inhibitors selected from 3% ethanol, 25 mM GITC, 6.25 μM hematin, 1.25 U/mL heparin, 0.01% SDS, 300 mM urea, 75 mM NaCl, and 250 ng gDNA. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 21.1.

TABLE 21.1
Activity Relative to SEQ ID NO: 2176

| SEQ ID | | FIOP Covid RT-qPCR with |
|---|---|---|
| NO: | Amino Acid Differences | Inhibitor(s) |
| (nt/aa) | (Relative to SEQ ID NO: 2176) | (Relative to SEQ ID NO: 2176)¹ |
| 2175/2176 | | + |
| 2863/2864 | Q543V | +++ |
| 2601/2602 | A530D | +++ |
| 2715/2716 | A530E | +++ |
| 2629/2630 | E65K/H489R/Q543A | +++ |
| 2669/2670 | S748G | +++ |
| 2499/2500 | E65N | +++ |
| 2561/2562 | R340T | +++ |
| 2623/2624 | A530T | +++ |
| 2617/2618 | S659T | +++ |
| 2941/2942 | R268Q | ++ |
| 2581/2582 | H489R/L658E | ++ |
| 2361/2362 | E721R | ++ |
| 2703/2704 | K802C | ++ |
| 2449/2450 | E406L | ++ |
| 2549/2550 | E466M | ++ |
| 2679/2680 | L666W | ++ |
| 2593/2594 | E109H | ++ |
| 2899/2900 | R269G | ++ |
| 2403/2404 | E312Q | ++ |
| 2229/2230 | R45S/A699Q/S748G/V749K | ++ |
| 2865/2866 | H489R | ++ |
| 2501/2502 | L666S | ++ |
| 2707/2708 | R605K | ++ |
| 2417/2418 | A451G | ++ |
| 2595/2596 | E481S | ++ |
| 2479/2480 | A560S | ++ |
| 2281/2282 | R45S/R268Q/H489R/P557R/S748G/V749K | ++ |
| 2453/2454 | R268G | ++ |
| 2913/2914 | L666E | ++ |
| 2937/2938 | E814T | ++ |
| 2949/2950 | N494S | ++ |
| 2389/2390 | P510R/S748G | ++ |
| 2769/2770 | E475H | ++ |
| 2239/2240 | L331S/R605K | ++ |
| 2553/2554 | A699Q | ++ |
| 2309/2310 | R45S/E475H/P557R/S748G/V749K | ++ |
| 2325/2326 | R45S/A451G | ++ |
| 2943/2944 | P510R | ++ |
| 2401/2402 | G431T | ++ |
| 2879/2880 | S221D | ++ |
| 2587/2588 | A243N | ++ |
| 2315/2316 | S748G/V749K | ++ |
| 2273/2274 | R45S/R268G/E312Q/R605K/S748G/V749K/E814T | ++ |
| 2541/2542 | E721A | ++ |
| 2227/2228 | Q543V/A699S/E814T | ++ |
| 2641/2642 | K802R | ++ |
| 2945/2946 | R358N/A467N | ++ |
| 2231/2232 | R45L/S621R | ++ |
| 2773/2774 | F81W | ++ |
| 2241/2242 | T597N | ++ |
| 2547/2548 | E475C | ++ |
| 2299/2300 | R268Q/E312Q/L331A/A423E/A451G/P557R/R605K | ++ |
| 2371/2372 | R352Q | ++ |
| 2365/2366 | P300T | ++ |
| 2443/2444 | A94K | ++ |
| 2635/2636 | V749K | + |
| 2529/2530 | P557R/R605K/A699Q/E721R | + |
| 2319/2320 | R268Q/E312Q/L331A/R340T/A699Q | + |
| 2827/2828 | L301G | + |
| 2427/2428 | T597G | + |
| 2939/2940 | E696Q | + |
| 2429/2430 | K57R | + |
| 2533/2534 | L331A | + |
| 2751/2752 | P300S | + |
| 2251/2252 | R45S/H489R/P557R/V749K | + |
| 2875/2876 | P557R | + |
| 2517/2518 | P302L/R340T/H489R/N494S/P510R/S748G/E814T | + |
| 2887/2888 | E304G/H489R | + |
| 2289/2290 | R340T/H489R/A699Q | + |
| 2583/2584 | R340L | + |
| 2287/2288 | P302V/N494V/A525R | + |
| 2915/2916 | A423E | + |
| 2471/2472 | A463W | + |
| 2261/2262 | R268Q/A423E/G431T/Q543A/P557R/R605K/E814T | + |
| 2253/2254 | H489R/P510R | + |
| 2245/2246 | R45S/E65N/A243N/R268Q | + |

¹Levels of increased activity were determined relative to the reference polypeptide of SEQ ID NO: 2176, and defined as follows: “+” > 0.85; “++” > 1.15; and “+++” > 2.

### Example 22

### Improvements Over SEQ ID NO: 2310 in Enzyme Activity

SEQ ID NO: 2310 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR reaction as described below.

For each variant tested, a 15 μL reaction mixture comprising 375 copies of SARS-CoV2 RNA fragment (Catalog #>102019, Twist) 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture; ThermoFisher), RT buffer (50 mM Tris-HCl, 73.1 mM KCl, 3.5 mM MgCl₂), 1.25 ng/uL of CODEX® HiTemp reverse transcriptase (Codexis), and 5 vol % HTP lysate of each variant. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 22.1.

TABLE 22.1
Activity Relative to SEQ ID NO: 2310

| SEQ ID NO: | Amino Acid Differences | Covid RT-qPCR |
|---|---|---|
| (nt/aa) | (Relative to SEQ ID NO: 2310) | (Relative to SEQ ID NO: 2310)¹ |
| 2309/2310 | | + |
| 2951/2952 | Q498T | ++++ |
| 2953/2954 | N494V/R605K/A699Q | ++++ |
| 2955/2956 | F484V | ++++ |
| 2957/2958 | E65K/F685R | ++++ |
| 2959/2960 | S524D | ++++ |
| 2961/2962 | F685K/A714T | ++++ |
| 2963/2964 | R268G/A423E/R478H/H489R/A525R/R605K/A699Q | ++++ |
| 2965/2966 | R358N/A467N/H489R/A699Q | ++++ |
| 2967/2968 | A467N/P510R/A699Q/E814T | ++++ |
| 2969/2970 | P510R/A699Q/E814T | ++++ |
| 2971/2972 | R268Q/H489R/A699Q | ++++ |
| 2973/2974 | A467N/P510R/A699S/E814T | ++++ |
| 2975/2976 | R268Q/P510R/A699Q/E814T | ++++ |
| 2977/2978 | A526Q | ++++ |
| 2979/2980 | S659G/A699Q | ++++ |
| 2981/2982 | A467N/H489R/A699Q | ++++ |
| 2983/2984 | A467N/H489R/E814T | ++++ |
| 2985/2986 | H489R/A699Q | ++++ |
| 2987/2988 | R268Q/Q543V/A699Q | ++++ |
| 2989/2990 | A467N/A699S | ++++ |
| 2991/2992 | A467N/P510R/A699Q | ++++ |
| 2993/2994 | R268Q/A467N/H489R/A699Q | ++++ |
| 2995/2996 | Q543V/A699Q/E814T | ++++ |
| 2997/2998 | R268Q/A467N | ++++ |
| 2999/3000 | E312Q/P510R/R605K | +++ |
| 3001/3002 | A699Q/E814T | +++ |
| 3003/3004 | R268Q/A699Q | +++ |
| 3005/3006 | Q498M | +++ |
| 3007/3008 | P510R | +++ |
| 3009/3010 | A467N/P510R/E814T | +++ |
| 3011/3012 | R268Q/P510R | +++ |
| 3013/3014 | H489R/P510R/A525R/R605K/A699Q | +++ |
| 3015/3016 | A699Q | +++ |
| 3017/3018 | A467N/A699Q | +++ |
| 3019/3020 | A423E/E443D/A451G/N494S/P510R | +++ |
| 3021/3022 | A467N/H489R/P510R/Q543V/A699S/E814T | +++ |
| 3023/3024 | R268G/S621R | +++ |
| 3025/3026 | R268Q/A467N/H489R | +++ |
| 3027/3028 | P510R/A699Q | +++ |
| 3029/3030 | K227E/L331A/A423E/R605K | +++ |
| 3031/3032 | P18H/R268Q/P302V/E312Q/R605K/S659G/A699Q | +++ |
| 3033/3034 | R358N/A467N/A699Q/E814T | +++ |
| 3035/3036 | S524Q | +++ |
| 3037/3038 | R358N/A467N/A699Q | +++ |
| 3039/3040 | K227E/A423E | +++ |
| 3041/3042 | S45L/K227E/P302L/A423E/P510R/R605K/S621R/A699Q | +++ |
| 3043/3044 | L331S/A423E | +++ |
| 3045/3046 | P510R/A699S/E814T | +++ |
| 3047/3048 | E312Q/A423E/S621R | +++ |
| 3049/3050 | S45L/R268Q/P302L/A423E/N494S/S621R/A699S | +++ |
| 3051/3052 | A423E/N494S | +++ |
| 3053/3054 | K227E/R268G/A423E/S621R | +++ |
| 3055/3056 | P302L/A423E/R605K/S621R | +++ |
| 3057/3058 | A451G/P510R | +++ |
| 3059/3060 | R268G/A423E/P510R/R605K | +++ |
| 3061/3062 | A423E/H489R/N494S/E529D/R605K/S621R/E696Q/A699Q | +++ |
| 3063/3064 | R268Q/R340T/A423E/E443D/A525R/S621R/S659G | +++ |
| 3065/3066 | R268Q/H489R/N494S/R605K/A699Q | +++ |
| 3067/3068 | L531V | +++ |
| 3069/3070 | R268Q/A699S | +++ |
| 3071/3072 | K227E/L331S | +++ |
| 3073/3074 | K227E/A699S | +++ |
| 3075/3076 | L503I | +++ |
| 3077/3078 | A684P | +++ |
| 3079/3080 | K227E/E312Q/H489R/N494S/S621R | +++ |
| 3081/3082 | K227E/L331S/R605K/S659G | +++ |
| 3083/3084 | S45L/N494V/A525R/S621R/E696Q/A699Q | +++ |
| 3085/3086 | A467N/H489R | +++ |
| 3087/3088 | K227E/R340T/S621R | +++ |
| 3089/3090 | V527R | +++ |
| 3091/3092 | H489R/A699S | +++ |
| 3093/3094 | K227E | +++ |
| 3095/3096 | K227E/P302L | +++ |
| 3097/3098 | R268Q/A467N/H489R/P510R/A699Q | +++ |
| 3099/3100 | R268Q/R605K/S621R/E721R | +++ |
| 3101/3102 | L331A/A525R/R605K/A699Q | +++ |
| 3103/3104 | A467N/H489R/A699S/E814T | +++ |
| 3105/3106 | S45L/R340T/A423E | +++ |
| 3107/3108 | K540S | +++ |
| 3109/3110 | P302L/Q543V/S621R/E696Q/A699Q | +++ |
| 3111/3112 | R605K/S621R/S659G/E696Q/A699S | +++ |
| 3113/3114 | S45L/K227E/E312Q/A423E/A525R/R605K/A699Q | +++ |
| 3115/3116 | A423E/A451G/R605K/S659G/A699Q | +++ |
| 3117/3118 | F685R | +++ |
| 3119/3120 | Q543V/A699Q | +++ |
| 3121/3122 | R268G/A423E/A699Q | +++ |
| 3123/3124 | T673K | +++ |
| 3125/3126 | F685K | +++ |
| 3127/3128 | K227E/R340T/A423E/R605K | +++ |
| 3129/3130 | S45L/L331A/S621R | +++ |
| 3131/3132 | R268Q/A423E/R605K/A699S | +++ |
| 3133/3134 | R605K | +++ |
| 3135/3136 | A423E/R605K/S621R | +++ |
| 3137/3138 | S621R | +++ |
| 3139/3140 | S45L/E696Q/A699Q | ++ |
| 3141/3142 | L331A/R605K/A699Q | ++ |
| 3143/3144 | K227E/R268Q/R340T/A451G | ++ |
| 3145/3146 | A467N | ++ |
| 3147/3148 | L331A/A423E/V527F | ++ |
| 3149/3150 | R340T/A451G/N494V/A525R | ++ |
| 3151/3152 | P510R/R605K/S659G | ++ |
| 3153/3154 | R268Q/A423E/H489R/N494S/R605K/S659G | ++ |
| 3155/3156 | R268Q/A467N/H489R/A699S/E814T | ++ |
| 3157/3158 | S45L/K227E/R268Q/A423E/H489R/P510R/R605K | ++ |
| 3159/3160 | A577S/R614C | ++ |
| 3161/3162 | E312Q/L331A/A451G/S621R/E696Q/A699Q | ++ |
| 3163/3164 | K227E/Q543V/S659G/A699S | ++ |
| 3165/3166 | K227E/A423E/A451G | ++ |
| 3167/3168 | R268G/P302L/A423E/R605K | ++ |
| 3169/3170 | S45L/R605K | ++ |
| 3171/3172 | R268Q/A525R/R605K | ++ |
| 3173/3174 | P302L/A423E | ++ |
| 3175/3176 | H489R | ++ |
| 3177/3178 | R268Q/A525R | ++ |
| 3179/3180 | Q543V/A699S/E814T | ++ |
| 3181/3182 | I702K | ++ |
| 3183/3184 | L542R | ++ |
| 3185/3186 | R268Q/R340T/A423E/S621R | ++ |
| 3187/3188 | R605K/S621R/E696Q/A699S | ++ |
| 3189/3190 | K227E/R605K/E721R | ++ |
| 3191/3192 | H489R/A699Q/E814T | ++ |
| 3193/3194 | R268Q/R340T/A525R/R605K | ++ |
| 3195/3196 | R268G/R340T | ++ |
| 3197/3198 | L569V | ++ |
| 3199/3200 | N494S/S659G | ++ |
| 3201/3202 | S45L/R268Q/E443D/A525R/E696Q | ++ |
| 3203/3204 | R268Q/A467N/P510R/A699S/E814T | ++ |
| 3205/3206 | L331S/A451G | ++ |
| 3207/3208 | H489R/N494S/R605K/E721R | ++ |
| 3209/3210 | P302L/R605K/S621R/E721R | ++ |
| 3211/3212 | L547M | ++ |
| 3213/3214 | A423E/A525R | ++ |
| 3215/3216 | R268Q/Q543V | + |
| 3217/3218 | R268Q/A423E | + |
| 3219/3220 | R268Q/P302L/R340T/R358N/R605K/A699Q | + |
| 3221/3222 | K227E/A525R | + |
| 3223/3224 | I716R | + |
| 3225/3226 | N494S/A699S | + |
| 3227/3228 | S45L/L331A/H489R | + |
| 3229/3230 | K227E/A451G/A525R/S621R/S659G/A699Q/E721R | + |

¹Levels of increased activity were determined relative to the reference polypeptide of SEQ ID NO: 2310, and defined as follows: “+” > 0.85; “++” > 1.15; “+++” > 3; and “++++” > 10.

### Example 23

### Improvements Over SEQ ID NO: 2310 in Enzyme Activity

SEQ ID NO: 2310 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened a RT-qPCR reaction, as described below.

For each variant tested, a 15 μL reaction mixture comprising 375 copies of non-infectious Influenza B (Inf-B) RNA fragment (Twist #>CAT_103003_Twist Synthetic Influenza B RNA Control, synthetic), and 500 nM INFB primers (SEQ ID NOS: 3259 and 3260; CAT #10011562 and CAT #10011563; IDT), 125 nM INFB (YAK) labeled probe (SEQ ID NO: 3261; CAT #10011564, IDT), 2.5× CODEX® HiTemp reaction buffer (Codexis) diluted to 1×, 1.25 ng/uL of CODEX® HiTemp reverse transcriptase (Codexis), and 2.5 vol % HTP lysate of each variant. Then, qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 23.1.

TABLE 23.1
Activity Relative to SEQ ID NO: 2310

| | | FIOP InfB RT-qPCR |
|---|---|---|
| SEQ ID NO: | Amino Acid Differences | no Inh |
| (nt/aa) | (Relative to SEQ ID NO: 2310) | (Relative to SEQ ID NO: 2310)¹ |
| 2309/2310 | | + |
| 3059/3060 | R268G/A423E/P510R/R605K | +++ |
| 3011/3012 | R268Q/P510R | +++ |
| 3231/3232 | R268Q/A467N/H489R/P510R/A699Q | +++ |
| 2953/2954 | N494V/R605K/A699Q | +++ |
| 2967/2968 | A467N/P510R/A699Q/E814T | +++ |
| 3053/3054 | K227E/R268G/A423E/S621R | +++ |
| 2971/2972 | R268Q/H489R/A699Q | +++ |
| 3157/3158 | S45L/K227E/R268Q/A423E/H489R/P510R/R605K | +++ |
| 2975/2976 | R268Q/P510R/A699Q/E814T | +++ |
| 3021/3022 | A467N/H489R/P510R/Q543V/A699S/E814T | +++ |
| 2993/2994 | R268Q/A467N/H489R/A699Q | +++ |
| 3023/3024 | R268G/S621R | +++ |
| 3045/3046 | P510R/A699S/E814T | +++ |
| 2969/2970 | P510R/A699Q/E814T | +++ |
| 3147/3148 | L331A/A423E/V527F | +++ |
| 2991/2992 | A467N/P510R/A699Q | ++ |
| 3233/3234 | P510R | ++ |
| 3099/3100 | R268Q/R605K/S621R/E721R | ++ |
| 2997/2998 | R268Q/A467N | ++ |
| 2999/3000 | E312Q/P510R/R605K | ++ |
| 3149/3150 | R340T/A451G/N494V/A525R | ++ |
| 3025/3026 | R268Q/A467N/H489R | ++ |
| 2995/2996 | Q543V/A699Q/E814T | ++ |
| 3057/3058 | A451G/P510R | ++ |
| 3167/3168 | R268G/P302L/A423E/R605K | ++ |
| 2973/2974 | A467N/P510R/A699S/E814T | ++ |
| 3031/3032 | P18H/R268Q/P302V/E312Q/R605K/S659G/A699Q | ++ |
| 3151/3152 | P510R/R605K/S659G | ++ |
| 3131/3132 | R268Q/A423E/R605K/A699S | ++ |
| 2951/2952 | Q498T | ++ |
| 3235/3236 | R268Q/A699Q | ++ |
| 3013/3014 | H489R/P510R/A525R/R605K/A699Q | ++ |
| 3237/3238 | H489R/A699Q | ++ |
| 2987/2988 | R268Q/Q543V/A699Q | ++ |
| 3081/3082 | K227E/L331S/R605K/S659G | ++ |
| 3075/3076 | L503I | ++ |
| 3135/3136 | A423E/R605K/S621R | ++ |
| 3109/3110 | P302L/Q543V/S621R/E696Q/A699Q | ++ |
| 3161/3162 | E312Q/L331A/A451G/S621R/E696Q/A699Q | ++ |
| 3027/3028 | P510R/A699Q | ++ |
| 2959/2960 | S524D | ++ |
| 3119/3120 | Q543V/A699Q | ++ |
| 3239/3240 | H489R/A699Q/E814T | ++ |
| 3083/3084 | S45L/N494V/A525R/S621R/E696Q/A699Q | ++ |
| 3209/3210 | P302L/R605K/S621R/E721R | ++ |
| 3241/3242 | R268Q/P302L | ++ |
| 3127/3128 | K227E/R340T/A423E/R605K | ++ |
| 2955/2956 | F484V | ++ |
| 3061/3062 | A423E/H489R/N494S/E529D/R605K/S621R/E696Q/A699Q | ++ |
| 3243/3244 | R268Q | ++ |
| 3189/3190 | K227E/R605K/E721R | ++ |
| 3087/3088 | K227E/R340T/S621R | ++ |
| 2983/2984 | A467N/H489R/E814T | ++ |
| 3041/3042 | S45L/K227E/P302L/A423E/P510R/R605K/S621R/A699Q | ++ |
| 2981/2982 | A467N/H489R/A699Q | ++ |
| 3187/3188 | R605K/S621R/E696Q/A699S | ++ |
| 3171/3172 | R268Q/A525R/R605K | ++ |
| 3143/3144 | K227E/R268Q/R340T/A451G | ++ |
| 3245/3246 | L331S/R605K | + |
| 3247/3248 | A467N/A699Q | + |
| 3029/3030 | K227E/L331A/A423E/R605K | + |
| 3141/3142 | L331A/R605K/A699Q | + |
| 3249/3250 | A467N/A699Q/E814T | + |
| 2965/2966 | R358N/A467N/H489R/A699Q | + |
| 3033/3034 | R358N/A467N/A699Q/E814T | + |
| 3251/3252 | A467N/H489R | + |
| 3001/3002 | A699Q/E814T | + |
| 3063/3064 | R268Q/R340T/A423E/E443D/A525R/S621R/S659G | + |
| 3071/3072 | K227E/L331S | + |
| 3253/3254 | A423E | + |
| 3039/3040 | K227E/A423E | + |
| 3047/3048 | E312Q/A423E/S621R | + |
| 3079/3080 | K227E/E312Q/H489R/N494S/S621R | + |
| 3111/3112 | R605K/S621R/S659G/E696Q/A699S | + |
| 3055/3056 | P302L/A423E/R605K/S621R | + |
| 3255/3256 | H489R | + |
| 3035/3036 | S524Q | + |
| 3005/3006 | Q498M | + |
| 2989/2990 | A467N/A699S | + |
| 3137/3138 | S621R | + |
| 3163/3164 | K227E/Q543V/S659G/A699S | + |
| 3049/3050 | S45L/R268Q/P302L/A423E/N494S/S621R/A699S | + |
| 3257/3258 | L331A/R605K | + |
| 3173/3174 | P302L/A423E | + |
| 3095/3096 | K227E/P302L | + |
| 3113/3114 | S45L/K227E/E312Q/A423E/A525R/R605K/A699Q | + |
| 2977/2978 | A526Q | + |
| 3043/3044 | L331S/A423E | + |
| 3093/3094 | K227E | + |
| 3019/3020 | A423E/E443D/A451G/N494S/P510R | + |

¹Levels of increased activity were determined relative to the reference polypeptide of SEQ ID NO: 2310, and defined as follows: “+” > 0.85; “++” > 1.15; and “+++” > 1.5.

### Example 24

### Improvements Over SEQ ID NO: 2310 in Enzyme Activity and Inhibitor Tolerance

SEQ ID NO: 2310 was selected as the parent enzyme for the round of evolution and enzyme characterization described in this Example. Libraries of engineered genes were produced techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR reaction, as described below.

Each variant was assayed in a 15 μL reaction mixture comprising 375 copies of non-infectious Influenza B (Inf-B) RNA fragment (Twist #>CAT_103003_Twist Synthetic Influenza B RNA Control, synthetic), and 500 nM INFB primers (SEQ ID NOS: 3262 and 3263; CAT #10011562 and CAT #10011563; IDT), 125 nM INFB (YAK) labeled probe (SEQ ID NO: 3264; CAT #10011564, IDT), 2.5× CODEX® HiTemp reaction buffer (Codexis) diluted to 1×, 1.25 ng/uL of CODEX® HiTemp reverse transcriptase (Codexis), and 2.5 vol % HTP lysate of each variant. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). The reaction also comprised either a single inhibitor or a combination of 3% ethanol, 25 mM GITC, 5 μM hematin, 1.256 U/mL heparin, 0.005% SDS, 300 mM urea, 80 mM NaCl, and 250 ng gDNA. For each variant, the Fold Improvement over Positive Control (FIOP) was calculated by the final relative fluorescence of the sample divided by the relative fluorescence of the control. The results are shown in Table 24.1.

TABLE 24.1
Activity Relative to SEQ ID NO: 2310

| SEQ ID | | FIOP InfB RT-qPCR |
|---|---|---|
| NO: | Amino Acid Differences | with Inh |
| (nt/aa) | (Relative to SEQ ID NO: 2310) | (Relative to SEQ ID NO: 2310)¹ |
| 2309/2310 | | + |
| 3083/3084 | S45L/N494V/A525R/S621R/E696Q/A699Q | +++ |
| 3109/3110 | P302L/Q543V/S621R/E696Q/A699Q | +++ |
| 3061/3062 | A423E/H489R/N494S/E529D/R605K/S621R/E696Q/A699Q | +++ |
| 3149/3150 | R340T/A451G/N494V/A525R | +++ |
| 2971/2972 | R268Q/H489R/A699Q | +++ |
| 3099/3100 | R268Q/R605K/S621R/E721R | +++ |
| 2993/2994 | R268Q/A467N/H489R/A699Q | +++ |
| 3021/3022 | A467N/H489R/P510R/Q543V/A699S/E814T | +++ |
| 3011/3012 | R268Q/P510R | +++ |
| 3013/3014 | H489R/P510R/A525R/R605K/A699Q | +++ |
| 3157/3158 | S45L/K227E/R268Q/A423E/H489R/P510R/R605K | +++ |
| 3131/3132 | R268Q/A423E/R605K/A699S | +++ |
| 2967/2968 | A467N/P510R/A699Q/E814T | +++ |
| 3189/3190 | K227E/R605K/E721R | +++ |
| 2953/2954 | N494V/R605K/A699Q | +++ |
| 3167/3168 | R268G/P302L/A423E/R605K | +++ |
| 2981/2982 | A467N/H489R/A699Q | +++ |
| 2997/2998 | R268Q/A467N | +++ |
| 3231/3232 | R268Q/A467N/H489R/P510R/A699Q | +++ |
| 2999/3000 | E312Q/P510R/R605K | +++ |
| 3025/3026 | R268Q/A467N/H489R | +++ |
| 3237/3238 | H489R/A699Q | +++ |
| 3119/3120 | Q543V/A699Q | +++ |
| 2969/2970 | P510R/A699Q/E814T | +++ |
| 3135/3136 | A423E/R605K/S621R | +++ |
| 3031/3032 | P18H/R268Q/P302V/E312Q/R605K/S659G/A699Q | ++ |
| 3233/3234 | P510R | ++ |
| 3081/3082 | K227E/L331S/R605K/S659G | ++ |
| 3147/3148 | L331A/A423E/V527F | ++ |
| 2995/2996 | Q543V/A699Q/E814T | ++ |
| 3161/3162 | E312Q/L331A/A451G/S621R/E696Q/A699Q | ++ |
| 2973/2974 | A467N/P510R/A699S/E814T | ++ |
| 3045/3046 | P510R/A699S/E814T | ++ |
| 3243/3244 | R268Q | ++ |
| 2983/2984 | A467N/H489R/E814T | ++ |
| 3239/3240 | H489R/A699Q/E814T | ++ |
| 3041/3042 | S45L/K227E/P302L/A423E/P510R/R605K/S621R/A699Q | + |

¹Levels of increased activity were determined relative to the reference polypeptide of SEQ ID NO: 2310, and defined as follows: “+” > 0.85; “++” > 1.15; and “+++” > 1.5.

### Example 25

### Improvements Over SEQ ID NO: 2310 in Enzyme Activity and Inhibitor Tolerance

SEQ ID NO: 2310 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR reaction, as described below.

Each variant was tested in a 15 μL reaction mixture comprising 75 copies of SARS-CoV2 RNA fragment (CAT_10006625_2019-nCoV_N_Positive Control; IDT), Twist CAT #102019, containing the nucleocapsid gene, 500 nM NI primers (SEQ ID NOS: 3262 and 3263), 125 nM FAM labeled probe (SEQ ID NO: 3264) (CDC EUA assay, 2019-nCoVEUA-01; IDT), 2.5× CODEX® HiTemp reaction buffer (Codexis) diluted to 1×, 1.25 ng/uL of CODEX® HiTemp reverse transcriptase (Codexis), 2.5 vol % HTP lysate of each variant. The reaction also comprised either a single inhibitor or a combination of two inhibitors selected from 3% ethanol, 25 mM GITC, 10 μM hematin, 2.5 U/mL heparin, 0.01% SDS, 300 mM urea, 135 mM NaCl, and 250 ng gDNA. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 25.1.

TABLE 25.1
Activity Relative to SEQ ID NO: 2310

| SEQ ID NO: | Amino Acid Differences | FIOP Covid RT-qPCR with Inh |
|---|---|---|
| (nt/aa) | (Relative to SEQ ID NO: 2310) | (Relative to SEQ ID NO: 2310)¹ |
| 2309/2310 | | + |
| 3237/3238 | H489R/A699Q | ++ |
| 2971/2972 | R268Q/H489R/A699Q | + |
| 3031/3032 | P18H/R268Q/P302V/E312Q/R605K/S659G/A699Q | + |
| 3117/3118 | F685R | + |
| 3099/3100 | R268Q/R605K/S621R/E721R | + |
| 3025/3026 | R268Q/A467N/H489R | + |
| 2961/2962 | F685K/A714T | + |
| 3125/3126 | F685K | + |
| 2983/2984 | A467N/H489R/E814T | + |
| 2997/2998 | R268Q/A467N | + |
| 3131/3132 | R268Q/A423E/R605K/A699S | + |
| 2957/2958 | E65K/F685R | + |
| 3243/3244 | R268Q | + |
| 2999/3000 | E312Q/P510R/R605K | + |
| 3239/3240 | H489R/A699Q/E814T | + |
| 2995/2996 | Q543V/A699Q/E814T | + |
| 2993/2994 | R268Q/A467N/H489R/A699Q | + |
| 2981/2982 | A467N/H489R/A699Q | + |
| 3135/3136 | A423E/R605K/S621R | + |
| 3081/3082 | K227E/L331S/R605K/S659G | + |
| 3189/3190 | K227E/R605K/E721R | + |
| 3109/3110 | P302L/Q543V/S621R/E696Q/A699Q | + |
| 3167/3168 | R268G/P302L/A423E/R605K | + |
| 3157/3158 | S45L/K227E/R268Q/A423E/H489R/P510R/R605K | + |
| 3161/3162 | E312Q/L331A/A451G/S621R/E696Q/A699Q | + |
| 3045/3046 | P510R/A699S/E814T | + |
| 3119/3120 | Q543V/A699Q | + |
| 3233/3234 | P510R | + |
| 3183/3184 | L542R | + |
| 3011/3012 | R268Q/P510R | + |
| 3021/3022 | A467N/H489R/P510R/Q543V/A699S/E814T | + |
| 3041/3042 | S45L/K227E/P302L/A423E/P510R/R605K/S621R/A699Q | + |
| 2973/2974 | A467N/P510R/A699S/E814T | + |
| 2967/2968 | A467N/P510R/A699Q/E814T | + |

¹Levels of increased activity were determined relative to the reference polypeptide of SEQ ID NO: 2310, and defined as follows: “+” > 0.85; and “++” > 1.15.

### Example 26

### Improvements Over SEQ ID NO: 2994 in Enzyme Activity

SEQ ID NO: 2994 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR reaction as described below.

For each variant tested, a 15 μL reaction mixture comprising 375 copies of SARS-CoV2 RNA fragment (CAT_10006625_2019-nCoV_N_Positive Control; IDT), Twist CAT #102019, containing the nucleocapsid gene, 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture; ThermoFisher), RT buffer (50 mM Tris-HCl, 73.1 mM KCl, 3.5 mM MgCl₂), 1.25 ng/uL of CODEX® HiTemp reverse transcriptase (Codexis), and 5 vol % HTP lysate of each variant. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 26.1.

TABLE 26.1
Activity Relative to SEQ ID NO: 2994

| SEQ ID NO: | Amino Acid Differences | FIOP Covid-19 RT-qPCR |
|---|---|---|
| (nt/aa) | (Relative to SEQ ID NO: 2994) | (Relative to SEQ ID NO: 2994)¹ |
| 3267/3268 | S10I | ++ |
| 3269/3270 | G427L | ++ |
| 3271/3272 | D346P | ++ |
| 3273/3274 | S10I | ++ |
| 3275/3276 | H4G | ++ |
| 3277/3278 | P122R | ++ |
| 3279/3280 | L436E | ++ |
| 3281/3282 | L436K | ++ |
| 3283/3284 | G349P | ++ |
| 3285/3286 | S688G | ++ |
| 3287/3288 | D99T | ++ |
| 3289/3290 | S659R | ++ |
| 3291/3292 | S659R | ++ |
| 3293/3294 | L426W | ++ |
| 3295/3296 | K517I | ++ |
| 3297/3298 | G427C | ++ |
| 3299/3300 | A105T | ++ |
| 3301/3302 | E690G | ++ |
| 3303/3304 | A662W | ++ |
| 3305/3306 | L426R/L798F | ++ |
| 3307/3308 | K565R | ++ |
| 3309/3310 | G11L | ++ |
| 3311/3312 | A105F | ++ |
| 3313/3314 | A76S | ++ |
| 3315/3316 | R485N | ++ |
| 3317/3318 | R485D | ++ |
| 3319/3320 | A810N | ++ |
| 3321/3322 | W177C | ++ |
| 3323/3324 | H6F | ++ |
| 3325/3326 | Q601S | ++ |
| 3327/3328 | R270P | ++ |
| 3329/3330 | G313S | ++ |
| 3331/3332 | I832Y | ++ |
| 3333/3334 | L370Y | ++ |
| 3335/3336 | H4G | ++ |
| 3337/3338 | K77R | ++ |
| 3339/3340 | A662T | ++ |
| 3341/3342 | L370D | ++ |
| 3343/3344 | G427E | ++ |
| 3345/3346 | V12W | ++ |
| 3347/3348 | E356V | ++ |
| 3349/3350 | E239I | ++ |
| 3351/3352 | E209V | ++ |
| 3353/3354 | H4A | ++ |
| 3355/3356 | W177L | ++ |
| 3357/3358 | A744G/H793P | ++ |
| 3359/3360 | Q642E/G692D | ++ |
| 3361/3362 | A423S | ++ |
| 3363/3364 | P48V | ++ |
| 3365/3366 | P445H | ++ |
| 3367/3368 | L426W | ++ |
| 3369/3370 | E167D | + |
| 3371/3372 | Q268S | + |
| 3373/3374 | A662F | + |
| 3375/3376 | D346R | + |
| 3377/3378 | G349V | + |
| 3379/3380 | N574Q | + |
| 3381/3382 | H6G | + |
| 3383/3384 | L426A | + |
| 3385/3386 | E533C | + |
| 3387/3388 | S688C | + |
| 3389/3390 | K514S | + |
| 3391/3392 | G427A | + |
| 3393/3394 | H6R | + |
| 3395/3396 | Q50H | + |
| 3397/3398 | A423G | + |
| 3399/3400 | A662Q | + |
| 3401/3402 | K77S | + |
| 3403/3404 | N574L | + |
| 3405/3406 | H4A | + |
| 3407/3408 | A662H | + |
| 3409/3410 | R485C | + |
| 3411/3412 | G11E | + |
| 3413/3414 | P14E/E167D | + |
| 3415/3416 | E167D | + |
| 3417/3418 | G427R | + |
| 3419/3420 | D346H | + |
| 3421/3422 | R448V/H793P | + |
| 3423/3424 | I832L | + |
| 3425/3426 | P14E/Q50H | + |
| 3427/3428 | R448S | + |
| 3429/3430 | P122S | + |
| 3431/3432 | G349R | + |
| 3433/3434 | A744L | + |
| 3435/3436 | D346M | + |
| 3437/3438 | K217G | + |
| 3439/3440 | P14E/E167D/K249E/Q642E | + |
| 3441/3442 | A105E | + |
| 3443/3444 | Q642E | + |
| 3445/3446 | L426H | + |
| 3447/3448 | K459R | + |
| 3449/3450 | S10R | + |
| 3451/3452 | P14E/E167D/T725H | + |
| 3453/3454 | S688A | + |
| 3455/3456 | Y439F/L509V/L789I | + |
| 3457/3458 | S659G | + |
| 3459/3460 | R485Q | + |
| 3461/3462 | E482A | + |
| 3463/3464 | Y695K | + |
| 3465/3466 | H4T | + |
| 3467/3468 | P377G | + |
| 3469/3470 | G11R | + |
| 3471/3472 | K565R | + |
| 3473/3474 | K514R | + |
| 3475/3476 | L426G | + |
| 3477/3478 | K77M | + |
| 3479/3480 | K517T | + |
| 3481/3482 | A810D | + |
| 3483/3484 | S659G | + |
| 3485/3486 | P801T | + |
| 3487/3488 | D99S | + |
| 3489/3490 | P48M | + |
| 3491/3492 | P122G | + |
| 3493/3494 | G427W | + |
| 3495/3496 | G427M | + |
| 3497/3498 | A810F | + |
| 3499/3500 | P14E/E167D/P197K/E433K | + |
| 3501/3502 | S659T | + |
| 3503/3504 | L436S | + |
| 3505/3506 | G818F | + |
| 3507/3508 | P14E | + |
| 3509/3510 | P14E | + |
| 3511/3512 | T725H | + |
| 3513/3514 | L361G | + |
| 3515/3516 | P14E/P598K | + |
| 3517/3518 | E167D | + |
| 3519/3520 | S299F | + |
| 3521/3522 | A744C | + |
| 3523/3524 | R489S | + |
| 3525/3526 | E433K/R501K/T725H | + |
| 3527/3528 | V830M | + |
| 3529/3530 | W177C | + |
| 3531/3532 | G427R | + |
| 3533/3534 | F484V/A684P | + |
| 3535/3536 | E440G | + |
| 3537/3538 | S299E | + |
| 3539/3540 | P197K/Y695K | + |
| 3541/3542 | R485F | + |
| 3543/3544 | A810G | + |
| 3545/3546 | P14E | + |
| 3547/3548 | G818A | + |
| 3549/3550 | E209N | + |
| 3551/3552 | D247G | + |
| 3553/3554 | G11L | + |
| 3555/3556 | G427K | + |
| 3557/3558 | P14E/P95D/R501K/Q642E | + |
| 3559/3560 | P14E/E433K/P598K | + |
| 3561/3562 | R139D/E167D | + |
| 3563/3564 | T394R | + |
| 3565/3566 | D346M | + |
| 3567/3568 | R489S | + |
| 3569/3570 | A662V | + |
| 3571/3572 | K459R | + |
| 3573/3574 | L426Q | + |
| 3575/3576 | G818R | + |
| 3577/3578 | A662D | + |
| 3579/3580 | G818A | + |
| 3581/3582 | P14E/R139D/K249E/E433K | + |
| 3583/3584 | D346R | + |
| 3585/3586 | S299V | + |
| 3587/3588 | E533R | + |
| 3589/3590 | A423E | + |
| 3591/3592 | G427S | + |
| 3593/3594 | K444V | + |
| 3595/3596 | K249E/T725H | + |
| 3597/3598 | G313F | + |
| 3599/3600 | P18H/A684P | + |
| 3601/3602 | P18H/A684P | + |
| 3603/3604 | R448K | + |
| 3605/3606 | Q50H/P598K | + |
| 3607/3608 | R596V | + |
| 3609/3610 | E751G | + |
| 3611/3612 | R485S | + |
| 3613/3614 | H4R | + |
| 3615/3616 | P598K/T725H | + |
| 3617/3618 | K517R | + |
| 3619/3620 | S299R/A474V | + |
| 3621/3622 | H6V | + |
| 3623/3624 | Q268H | + |
| 3625/3626 | K517N | + |
| 3627/3628 | K249E/Y695K | + |
| 3629/3630 | H6G | + |
| 3631/3632 | T394G | + |
| 3633/3634 | P14E/E167D/Q642E | + |
| 3635/3636 | G818S | + |
| 3637/3638 | G404S | + |
| 3639/3640 | W177R | + |
| 3641/3642 | H29W | + |
| 3643/3644 | L446M/L811I | + |
| 3645/3646 | S621R/S659G | + |
| 3647/3648 | S299F | + |
| 3649/3650 | L436H | + |
| 3651/3652 | D99G | + |
| 3653/3654 | P14E/R139D/T725H | + |
| 3655/3656 | K227M | + |
| 3657/3658 | P122N | + |
| 3659/3660 | S45L/Q268G/S299N/P302L/S321T/S621R/S659G | + |
| | | + |
| 3661/3662 | P14E/E167D/Q642E | + |
| 3663/3664 | P14E/E167D/Q642E | + |
| 3665/3666 | S299V | + |
| 3667/3668 | G313S | + |
| 3669/3670 | G427S | + |
| 3671/3672 | E239L | + |
| 3673/3674 | H4G | + |
| 3675/3676 | P14E/E167D/Y695K | + |
| 3677/3678 | P14E/Q50H/P598K/Y695K | + |
| 3679/3680 | G818L | + |
| 3681/3682 | P95D/E167D/P197K/P598K/Y695K/T725H | + |
| 3683/3684 | G349N | + |
| 3685/3686 | S299G | + |
| 3687/3688 | P14E/K249E/Q642E | + |
| 3689/3690 | T394D | + |
| 3691/3692 | P598K/Y695K | + |
| 3693/3694 | P122I | + |
| 3695/3696 | T394C | + |
| 3697/3698 | G404S | + |
| 3699/3700 | G404V | + |
| 3701/3702 | A810C | + |
| 3703/3704 | L436G | + |
| 3705/3706 | K459F | + |
| 3707/3708 | A76W | + |
| 3709/3710 | R489W | + |
| 3711/3712 | N636G | + |
| 3713/3714 | A105L | + |
| 3715/3716 | P14E/E167D/Q642E | + |
| 3717/3718 | P302V | + |
| 3719/3720 | T725H | + |
| 3721/3722 | G818L | + |
| 3723/3724 | G818E | + |
| 3725/3726 | H4F | + |
| 3727/3728 | K565T | + |
| 3729/3730 | L436Y | + |
| 3731/3732 | G818A | + |
| 3733/3734 | S10Q | + |
| 3735/3736 | F484V | + |
| 3737/3738 | E356Q | + |
| 3739/3740 | P14E/P598K/Y695K | + |
| 3741/3742 | S659N | + |
| 3743/3744 | H6R | + |
| 3745/3746 | S659G | + |
| 3747/3748 | H29N | + |
| 3749/3750 | R448G | + |
| 3751/3752 | A76L | + |
| 3753/3754 | E167D/P197K | + |
| 3755/3756 | A662V | + |
| 3757/3758 | H29V | + |
| 3759/3760 | R139D/E433K/P598K | + |
| 3761/3762 | E167D/T725H | + |
| 3763/3764 | R139D/E167D/P197K/E433K/R501K/Q642E | + |
| 3765/3766 | H4G | + |
| 3767/3768 | H29M | + |
| 3769/3770 | P14E | + |
| 3771/3772 | G349W | + |
| 3773/3774 | Q642E | + |
| 3775/3776 | A684P | + |
| 3777/3778 | G141C | + |
| 3779/3780 | P18H/S524D/L547M | + |
| 3781/3782 | A423V | + |
| 3783/3784 | A105G | + |
| 3785/3786 | D99T | + |
| 3787/3788 | S299K | + |
| 3789/3790 | A423R | + |
| 3791/3792 | L370T | + |
| 3793/3794 | E167D/P197K | + |
| 3795/3796 | S45L/Q268G/P302L/S621R/E721R | + |
| 3797/3798 | G349T | + |
| 3799/3800 | I832F | + |
| 3801/3802 | S683K | + |
| 3803/3804 | G404V | + |
| 3805/3806 | P14E/P598K/Y695K/T725H | + |
| 3807/3808 | E167D/Q642E | + |
| 3809/3810 | T725H | + |
| 3811/3812 | G427T | + |
| 3813/3814 | A810G | + |
| 3815/3816 | P14E/E167D/P197K/Q642E/Y695K | + |
| 3817/3818 | G818S | + |
| 3819/3820 | T725H | + |
| 3821/3822 | P18H | + |
| 3823/3824 | D741S | + |
| 3825/3826 | P801S | + |
| 3827/3828 | P95D/K249E/P598K | + |
| 3829/3830 | D346T | + |
| 3831/3832 | R448Q | + |
| 3833/3834 | A91S | + |
| 3835/3836 | S299G | + |
| 3837/3838 | G427R | + |
| 3839/3840 | E167D/T725H | + |
| 3841/3842 | P14E/E167D/K249E/P598K/Y695K/T725H | + |
| 3843/3844 | V12D | + |
| 3845/3846 | E690G | + |
| 3847/3848 | P14E/E167D | + |
| 3849/3850 | P14E/P598K | + |
| 3851/3852 | L426G | + |
| 3853/3854 | L426V | + |
| 3855/3856 | A423F | + |
| 3857/3858 | K227S | + |
| 3859/3860 | Q543V/S621R/E721R | + |
| 3861/3862 | S621R | + |
| 3863/3864 | P302V/A423E/A451G/N494S/S621R | + |
| 3865/3866 | A525R/E529D/S621R | + |
| 3867/3868 | L374G | + |
| 3869/3870 | Q642E | + |
| 3871/3872 | P14E/P197K/K249E/Q642E | + |
| 3873/3874 | K565C | + |
| 3875/3876 | P14E/E167D/P197K/R501K/T725H | + |
| 3877/3878 | P14E/E167D/K249E/R501K/P598K/Q642E | + |
| 3879/3880 | P202G | + |
| 3881/3882 | R448T | + |
| 3883/3884 | P14E/R501K | + |
| 3885/3886 | D99L | + |
| 3887/3888 | K444V | + |
| 3889/3890 | S299E | + |
| 3891/3892 | A423E/A451G/S621R/E696Q | + |
| 3893/3894 | E356R | + |
| 3895/3896 | Y53Q | + |
| 3897/3898 | E440W | + |
| 3899/3900 | H4G | + |
| 3901/3902 | P18H/P302V | + |
| 3903/3904 | E239Y | + |
| 3905/3906 | P377L | + |
| 3907/3908 | Q50H/Q642E | + |
| 3909/3910 | H4T | + |
| 3911/3912 | P598K | + |
| 3913/3914 | R485M | + |
| 3915/3916 | K565S | + |
| 3917/3918 | E690R | + |
| 3919/3920 | A744L | + |
| 3921/3922 | E356A | + |
| 3923/3924 | P122L | + |
| 3925/3926 | P18H | + |
| 3927/3928 | K444T | + |
| 3929/3930 | H4V | + |
| 3931/3932 | K77H | + |
| 3933/3934 | T394M | + |
| 3935/3936 | D741G | + |
| 3937/3938 | Q268G | + |
| 3939/3940 | F484V | + |
| 3941/3942 | P14E/E167D/Y695K | + |
| 3943/3944 | P14E/Q642E/T725H | + |
| 3945/3946 | G404R | + |
| 3947/3948 | P18H | + |
| 3949/3950 | P18H/A684P | + |
| 3951/3952 | G818S | + |
| 3953/3954 | A744W | + |
| 3955/3956 | E356G | + |
| 3957/3958 | S659A | + |
| 3959/3960 | W177Q | + |
| 3961/3962 | E356G | + |
| 3963/3964 | H6N | + |
| 3965/3966 | P14E/K249E/P598K | + |
| 3967/3968 | V204L | + |
| 3969/3970 | Q50H/E433K/P598K | + |
| 3971/3972 | A105L | + |
| 3973/3974 | Q50H/Q642E | + |
| 3975/3976 | P14E/K249E/T725H | + |
| 3977/3978 | D461A | + |
| 3979/3980 | K565R | + |
| 3981/3982 | Q642E | + |
| 3983/3984 | A684P | + |
| 3985/3986 | S45L/L376F/L385F/T408A/N494V/A525R/S621R/E696Q | + |
| 3987/3988 | E167D/P197K/Q642E/Y695K/T725H | + |
| 3989/3990 | G404K | + |
| 3991/3992 | D247A | + |
| 3993/3994 | P14E/E167D/T725H | + |
| 3995/3996 | L436W | + |
| 3997/3998 | G818A | + |
| 3999/4000 | E167D | + |
| 4001/4002 | L547M | + |
| 4003/4004 | P18H/P302V/L547M | + |
| 4005/4006 | S299P | + |
| 4007/4008 | P598K/Q642E/Y695K | + |
| 4009/4010 | T673K | + |
| 4011/4012 | P598K | + |
| 4013/4014 | T394G | + |
| 4015/4016 | E690S | + |
| 4017/4018 | A91R | + |
| 4019/4020 | K217T | + |
| 4021/4022 | P14E/K249E/Q642E | + |
| 4023/4024 | Q268G/P302V/A451G/N494S/S621R | + |
| 4025/4026 | P14E/P95D | + |
| 4027/4028 | E209M | + |
| 4029/4030 | A744Q | + |
| 4031/4032 | K217R | + |
| 4033/4034 | P14E/K249E/E433K/T725H | + |
| 4035/4036 | Y695K | + |
| 4037/4038 | A76Y | + |
| 4039/4040 | P18H/L547M | + |
| 4041/4042 | L426A | + |
| 4043/4044 | E440C | + |
| 4045/4046 | E239W | + |
| 4047/4048 | P445G/L446V | + |
| 4049/4050 | R596A | + |
| 4051/4052 | Q50H/P598K/Y695K | + |
| 4053/4054 | G404C | + |
| 4055/4056 | A91W | + |
| 4057/4058 | E356I | + |
| 4059/4060 | P14E | + |
| 4061/4062 | P14E/P197K/Q642E | + |
| 4063/4064 | P95D/P598K | + |
| 4065/4066 | A91C | + |
| 4067/4068 | P14E/Q50H/A56V/P598K/T725H | + |
| 4069/4070 | E610A | + |
| 4071/4072 | G141C | + |
| 4073/4074 | G404C | + |
| 4075/4076 | N494V | + |
| 4077/4078 | E209G | + |
| 4079/4080 | S621R | + |
| 4081/4082 | D266E | + |
| 4083/4084 | L509A | + |
| 4085/4086 | A105S | + |
| 4087/4088 | Y695K | + |
| 4089/4090 | S621R | + |
| 4091/4092 | F484V | + |
| 4093/4094 | P18H | + |
| 4095/4096 | Q689G | + |
| 4097/4098 | Q50H/R501K | + |
| 4099/4100 | P18H/L547M | + |
| 4101/4102 | G313V | + |
| 4103/4104 | P18H/P302V/L531V/T673K/A684P | + |
| 4105/4106 | S299M | + |
| 4107/4108 | L426G | + |
| 4109/4110 | K517S | + |
| 4111/4112 | P18H/F484V/T673K/A684P | + |
| 4113/4114 | P95D/E167D/P197K/P598K/Q642E/Y695K | + |
| 4115/4116 | T673K | + |
| 4117/4118 | A423E/N494V/A525R | + |
| 4119/4120 | P14E/E167D/K249E/E433K/P598K/Q642E | + |
| 4121/4122 | R485T | + |
| 4123/4124 | A76S | + |
| 4125/4126 | P197K/Q642E/T725H | + |
| 4127/4128 | P598K/Q642E | + |
| 4129/4130 | Y53W | + |
| 4131/4132 | K459L | + |
| 4133/4134 | G313T | + |
| 4135/4136 | Q498M/A684P | + |
| 4137/4138 | S45L/A423E | + |
| 4139/4140 | E167D/Q642E | + |
| 4141/4142 | D99P | + |
| 4143/4144 | R485T | + |
| 4145/4146 | P302V/A451G/N494S | + |
| 4147/4148 | Y53A | + |
| 4149/4150 | S45L/A423E | + |
| 4151/4152 | P18H/F484V/T673K | + |
| 4153/4154 | P95D/K249E/E433K/Q642E | + |
| 4155/4156 | P18H | + |
| 4157/4158 | L291Q | + |
| 4159/4160 | P302V/A423E/S621R | + |
| 4161/4162 | L291Q/A684P | + |
| 4163/4164 | A744R | + |

¹Levels of increased activity were determined relative to the reference polypeptide of SEQ ID NO: 2994, and defined as follows: “+” > 0.85, and “++” > 1.2.

### Example 27

### Improvements Over SEQ ID NO: 2994 in Enzyme Activity

SEQ ID NO: 2994 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened a RT-qPCR reaction, as described below.

For each variant tested, a 15 μL reaction mixture comprising 375 copies of non-infectious Influenza B (Inf-B) RNA fragment (Twist #>CAT_103003_Twist Synthetic Influenza B RNA Control, synthetic), and 500 nM INFB primers (SEQ ID NOS: 3259 and 3260; CAT #10011562 and CAT #10011563; IDT), 125 nM INFB (YAK) labeled probe (SEQ ID NO: 3261; CAT #10011564, IDT), 1× CODEX® HiTemp reaction buffer (Codexis), 1.25 ng/uL of CODEX® HiTemp reverse transcriptase (Codexis), and 2.5 vol % HTP lysate of each variant. Then, RT-qPCR cycling (50° C. 5 min, 95° C. 2 min, 95° C. 5 sec, 66.7° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 27.

TABLE 27.1
Activity Relative to SEQ ID NO: 2994

| SEQ ID NO: | Amino Acid Differences | FIOP Inf-B RNA RT-qPCR |
|---|---|---|
| (nt/aa) | (Relative to SEQ ID NO: 2994) | (Relative to SEQ ID NO: 2994)¹ |
| 3389/3390 | K514S | +++ |
| 3455/3456 | Y439F/L509V/L789I | +++ |
| 3795/3796 | S45L/Q268G/P302L/S621R/E721R | +++ |
| 3311/3312 | A105F | +++ |
| 3375/3376 | D346R | +++ |
| 3411/3412 | G11E | +++ |
| 3301/3302 | E690G | +++ |
| 3421/3422 | R448V/H793P | +++ |
| 3385/3386 | E533C | +++ |
| 3779/3780 | P18H/S524D/L547M | +++ |
| 3659/3660 | S45L/Q268G/S299N/P302L/S321T/S621R/S659G | +++ |
| 3939/3940 | F484V | +++ |
| 3305/3306 | L426R/L798F | +++ |
| 3735/3736 | F484V | +++ |
| 3357/3358 | A744G/H793P | +++ |
| 3859/3860 | Q543V/S621R/E721R | +++ |
| 3491/3492 | P122G | +++ |
| 3349/3350 | E239I | +++ |
| 3599/3600 | P18H/A684P | +++ |
| 3339/3340 | A662T | +++ |
| 3287/3288 | D99T | +++ |
| 3533/3534 | F484V/A684P | +++ |
| 3335/3336 | H4G | +++ |
| 3403/3404 | N574L | +++ |
| 3297/3298 | G427C | +++ |
| 3303/3304 | A662W | +++ |
| 3775/3776 | A684P | +++ |
| 3461/3462 | E482A | +++ |
| 3457/3458 | S659G | +++ |
| 3277/3278 | P122R | +++ |
| 3477/3478 | K77M | +++ |
| 3423/3424 | I832L | +++ |
| 3291/3292 | S659R | +++ |
| 3377/3378 | G349V | +++ |
| 3601/3602 | P18H/A684P | +++ |
| 4103/4104 | P18H/P302V/L531V/T673K/A684P | +++ |
| 3295/3296 | K517I | +++ |
| 3479/3480 | K517T | +++ |
| 3285/3286 | S688G | +++ |
| 3269/3270 | G427L | +++ |
| 3485/3486 | P801T | +++ |
| 3351/3352 | E209V | +++ |
| 3445/3446 | L426H | +++ |
| 3497/3498 | A810F | +++ |
| 3503/3504 | L436S | ++ |
| 3387/3388 | S688C | ++ |
| 3427/3428 | R448S | ++ |
| 3347/3348 | E356V | ++ |
| 3393/3394 | H6R | ++ |
| 3453/3454 | S688A | ++ |
| 3473/3474 | K514R | ++ |
| 3891/3892 | A423E/A451G/S621R/E696Q | ++ |
| 3861/3862 | S621R | ++ |
| 3371/3372 | Q268S | ++ |
| 3289/3290 | S659R | ++ |
| 3363/3364 | P48V | ++ |
| 3469/3470 | G11R | ++ |
| 3381/3382 | H6G | ++ |
| 3487/3488 | D99S | ++ |
| 3275/3276 | H4G | ++ |
| 3391/3392 | G427A | ++ |
| 3355/3356 | W177L | ++ |
| 3271/3272 | D346P | ++ |
| 3691/3692 | P598K/Y695K | ++ |
| 3431/3432 | G349R | ++ |
| 3475/3476 | L426G | ++ |
| 3281/3282 | L436K | ++ |
| 3279/3280 | L436E | ++ |
| 3361/3362 | A423S | ++ |
| 3283/3284 | G349P | ++ |
| 3645/3646 | S621R/S659G | ++ |
| 3437/3438 | K217G | ++ |
| 3417/3418 | G427R | ++ |
| 3627/3628 | K249E/Y695K | ++ |
| 3293/3294 | L426W | ++ |
| 3481/3482 | A810D | ++ |
| 3507/3508 | P14E | ++ |
| 3515/3516 | P14E/P598K | ++ |
| 3425/3426 | P14E/Q50H | ++ |
| 3365/3366 | P445H | ++ |
| 3407/3408 | A662H | ++ |
| 3605/3606 | Q50H/P598K | ++ |
| 3819/3820 | T725H | ++ |
| 3369/3370 | E167D | ++ |
| 3663/3664 | P14E/E167D/Q642E | ++ |
| 3367/3368 | L426W | + |
| 3511/3512 | T725H | + |
| 3273/3274 | S10I | + |
| 3319/3320 | A810N | + |
| 3653/3654 | P14E/R139D/T725H | + |
| 3397/3398 | A423G | + |
| 3525/3526 | E433K/R501K/T725H | + |
| 3675/3676 | P14E/E167D/Y695K | + |
| 3413/3414 | P14E/E167D | + |
| 3471/3472 | K565R | + |
| 3681/3682 | P95D/E167D/P197K/P598K/Y695K/T725H | + |
| 3715/3716 | P14E/E167D/Q642E | + |
| 3299/3300 | A105T | + |
| 3793/3794 | E167D/P197K | + |

¹Levels of increased activity were determined relative to the reference polypeptide of SEQ ID NO: 2994, and defined as follows: “+” > 0.85; “++” > 1.2; and “+++” > 2.

### Example 28

### Improvements Over SEQ ID NO: 2994 in Enzyme Activity and Inhibitor Tolerance

SEQ ID NO: 2994 was selected as the parent enzyme for the round of evolution and enzyme characterization described in this Example. Libraries of engineered genes were produced using techniques such as saturation mutagenesis and recombination of previously identified beneficial mutations). The polypeptides encoded by each gene were produced in HTP as described in Example 2, and the soluble lysate was generated as described in Example 3. Each variant was screened in a RT-qPCR reaction, as described below.

Each variant was assayed in a 15 μL reaction mixture comprising 375 copies of non-infectious Influenza B (Inf-B) RNA fragment (Twist #>CAT_103003_Twist Synthetic Influenza B RNA Control, synthetic), and 500 nM INFB primers (SEQ ID NOS: 3262 and 3263; CAT #10011562 and CAT #10011563; IDT), 125 nM INFB (YAK) labeled probe (SEQ ID NO: 3264; CAT #10011564, IDT), 1× CODEX® HiTemp reaction buffer (Codexis), 1.25 ng/uL of CODEX® HiTemp reverse transcriptase (Codexis), and 2.5 vol % HTP lysate of each variant. Then, RT-qPCR cycling (50° C. 5 min, 95° C. 2 min, 95° C. 5 sec, 66.7° C. 30 sec, for 50 cycles) was conducted in a CFX384 Touch Real-Time PCR Detection System (BioRad). The reaction also comprised either a single inhibitor or a combination of 3% ethanol, 25 mM GITC, 5 μM hematin, 4.25 U/mL heparin, 0.007% SDS, 300 mM urea, 130 mM NaCl, and 250 ng gDNA. For each variant, the Fold Improvement over Positive Control (FIOP) was calculated by the final relative fluorescence of the sample divided by the relative fluorescence of the control. The results are shown in Table 28.1.

TABLE 28.1
Activity Relative to SEQ ID NO: 2994

| SEQ ID NO: | Amino Acid Differences | FIOP - Inf-B RT-qPCR With Inhibitors |
|---|---|---|
| (nt/aa) | (Relative to SEQ ID NO: 2994) | (Relative to SEQ ID NO: 2994)¹ |
| 3939/3940 | F484V | +++ |
| 3345/3346 | V12W | +++ |
| 3305/3306 | L426R/L798F | +++ |
| 3389/3390 | K514S | +++ |
| 3455/3456 | Y439F/L509V/L789I | +++ |
| 3357/3358 | A744G/H793P | +++ |
| 3385/3386 | E533C | +++ |
| 3301/3302 | E690G | +++ |
| 4039/4040 | P18H/L547M | +++ |
| 3421/3422 | R448V/H793P | ++ |
| 3375/3376 | D346R | ++ |
| 3489/3490 | P48M | ++ |
| 3311/3312 | A105F | ++ |
| 3491/3492 | P122G | ++ |
| 3411/3412 | G11E | ++ |
| 3379/3380 | N574Q | ++ |
| 3985/3986 | S45L/L376F/L385F/T408A/N494V/A525R/S621R/E696Q | ++ |
| 3865/3866 | A525R/E529D/S621R | ++ |
| 3795/3796 | S45L/Q268G/P302L/S621R/E721R | + |
| 3859/3860 | Q543V/S621R/E721R | + |
| 3659/3660 | S45L/Q268G/S299N/P302L/S321T/S621R/S659G | + |
| 3717/3718 | P302V | + |

¹Levels of increased activity were determined relative to the reference polypeptide of SEQ ID NO: 2994, and defined as follows: “+” > 0.85; “++” > 1.2; and “+++” > 2.

### Example 29

### Improvements Over SEQ ID NO: 2994 in Enzyme Activity and Inhibitor Tolerance

SEQ ID NO: 2994 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR reaction, as described below.

Each variant was tested in a 15 μL reaction mixture comprising 50 copies of SARS-CoV2 RNA fragment (CAT_10006625_2019-nCoV_N_Positive Control; IDT), Twist CAT #102019, containing the nucleocapsid gene, 500 nM N1 primers (SEQ ID NOS: 3262 and 3263), 125 nM FAM labeled probe (SEQ ID NO: 3264) (CDC EUA assay, 2019-nCoVEUA-01; IDT), 1× CODEX® HiTemp reaction buffer (Codexis), 1.25 ng/uL of CODEX® HiTemp reverse transcriptase (Codexis), and 5 vol % HTP lysate of each variant. The reaction also comprised either a single inhibitor or a combination of two inhibitors selected from 3% ethanol, 25 mM GITC, 10 μM hematin, 6.25 U/mL heparin, 0.01% SDS, 300 mM urea, 175 mM NaCl, and 250 ng gDNA. Then, RT-qPCR cycling (50° C. 5 min, 95° C. 2 min, 95° C. 5 sec, 60° C. 30 sec, for 50 cycles) was conducted in a CFX384 Touch Real-Time PCR Detection System (BioRad). For each variant, the Fold Improvement over Positive Control (FIOP) was calculated by the final relative fluorescence of the sample divided by the relative fluorescence of the control. The results are shown in Table 29.1. For each variant, the Fold Improvement over Positive Control (FIOP) was calculated by the final relative fluorescence of the sample divided by the relative fluorescence of the control. The results are shown in Table 29.1.

TABLE 29.1 Activity Relative to SEQ ID NO: 2994

| | | FIOP - Covid-19 RT-qPCR |
|---|---|---|
| SEQ ID NO: | Amino Acid Differences | With Inhibitors |
| (nt/aa) | (Relative to SEQ ID NO: 2994) | (Relative to SEQ ID NO: 2994)¹ |
| 3421/3422 | R448V/H793P | +++ |
| 4039/4040 | P18H/L547M | +++ |
| 3311/3312 | A105F | +++ |
| 3489/3490 | P48M | +++ |
| 3301/3302 | E690G | +++ |
| 3455/3456 | Y439F/L509V/L789I | +++ |
| 3385/3386 | E533C | +++ |
| 3357/3358 | A744G/H793P | +++ |
| 3389/3390 | K514S | +++ |
| 4001/4002 | L547M | +++ |
| 4161/4162 | L291Q/A684P | +++ |
| 3901/3902 | P18H/P302V | +++ |
| 4003/4004 | P18H/P302V/L547M | +++ |
| 3411/3412 | G11E | +++ |
| 3939/3940 | F484V | +++ |
| 3983/3984 | A684P | +++ |
| 3659/3660 | S45L/Q268G/S299N/P302L/S321T/S621R/S659G | ++ |
| 3947/3948 | P18H | ++ |
| 3717/3718 | P302V | ++ |
| 3375/3376 | D346R | ++ |
| 3985/3986 | S45L/L376F/L385F/T408A/N494V/A525R/S621R/E696Q | ++ |
| 3795/3796 | S45L/Q268G/P302L/S621R/E721R | ++ |
| 3305/3306 | L426R/L798F | ++ |
| 3345/3346 | V12W | ++ |
| 3735/3736 | F484V | ++ |
| 3491/3492 | P122G | + |
| 3925/3926 | P18H | + |
| 3379/3380 | N574Q | + |
| 3865/3866 | A525R/E529D/S621R | + |

¹Levels of increased activity were determined relative to the reference polypeptide of SEQ ID NO: 2994, and defined as follows: “+” > 0.85; “++” > 1.2; and “+++” > 2.

### Example 30

### Improvements Over SEQ ID NO: 3796 in Enzyme Sensitivity and Processivity (Activity)

SEQ ID NO: 3796 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR reaction, as described below.

Each variant was tested in a 15 μL reaction mixture comprising 10 copies of HeLa RNA fragment (Takara CAT #636543), primers Applied Biosystems Human GAPD (GAPDH) Endogenous Control (FAM™/MGB probe, non-primer limited) CAT #4352934E, 1× CODEX® HiTemp reaction buffer (Codexis), 1.25 ng/uL of CODEX® HiTemp reverse transcriptase (Codexis), and 2 vol % HTP lysate of each variant. RT-qPCR cycling (50° C. 5 min, 95° C. 2 min, 95° C. 3 sec, 60° C. 15 sec, for 50 cycles) was conducted in a CFX384 Touch Real-Time PCR Detection System (BioRad). For each variant, the quantification cycle at which fluorescence level is higher than the threshold, designated as “Cq,” was determined. The Cq Fold Improvement over Positive Control (FIOP) was calculated by the shift in Cq relative to the Cq value of the positive control. The results are shown in Table 30.1.

TABLE 30.1 Activity Relative to SEQ ID NO: 3796

| | | FIOP HeLa |
|---|---|---|
| | | RT-qPCR |
| | | (Relative |
| SEQ ID NO: | Amino Acid Differences | to SEQ |
| (nt/aa) | (Relative to SEQ ID NO: 3796) | ID NO: 3796)¹ |
| 4165/4166 | L625T | + |
| 4167/4168 | M770R | + |
| 4169/4170 | A805G | + |
| 4171/4172 | L615S | + |
| 4173/4174 | N675L | + |
| 4175/4176 | F484L | + |
| 4177/4178 | R489L | + |
| 4179/4180 | F491W | + |
| 4181/4182 | A423E/N424K/L547M | + |
| 4183/4184 | E533W | + |
| 4185/4186 | E546C | + |
| 4187/4188 | K249E/R448V/A451G/L547M/S659G/T725H | + |
| 4189/4190 | M1H/H2M/A149G | + |
| 4191/4192 | H535N | + |
| 4193/4194 | E690V | + |
| 4195/4196 | Q703R | + |
| 4197/4198 | H535S | + |
| 4199/4200 | N743A | + |
| 4201/4202 | T394D | + |
| 4203/4204 | D664M | + |
| 4205/4206 | E372S | + |
| 4207/4208 | L291Q/S321T/A423E/T725H | + |
| 4209/4210 | P18H/L291Q/A423E/A525R/E529D | + |
| 4211/4212 | G600A | + |
| 4213/4214 | L625I | + |
| 4215/4216 | Q498C | + |
| 4217/4218 | P18H/F484V | + |
| 4219/4220 | R139D/K249E/L291Q/S321T/F484V/Y695K/T725H | + |
| 4221/4222 | A423E/E529D/A684P | + |
| 4223/4224 | A423E/F484V/T673K | + |
| 4225/4226 | V472T | + |
| 4227/4228 | P300G | + |
| 4229/4230 | E697S | + |
| 4231/4232 | A105F/R139D/L291Q/L302V/A423E/F484V/L547M/T725H | + |
| 4233/4234 | A105F/S321T/R501K | + |
| 4235/4236 | N200S/A577G | + |
| 4237/4238 | R501L | + |
| 4239/4240 | M476S | + |
| 4241/4242 | V746- | + |
| 4243/4244 | P18H/K249E | + |
| 4245/4246 | P18H/A423E | + |
| 4247/4248 | L302G | + |
| 4249/4250 | V819W | + |
| 4251/4252 | R256S | + |
| 4253/4254 | V819R | + |
| 4255/4256 | D247Y/A423E/R448V | + |
| 4257/4258 | P18H/K249E/R448V/F484V/T673K | + |
| 4259/4260 | A105F/S321T/E433K/A744G | + |
| 4261/4262 | V819S | + |
| 4263/4264 | A753R | + |
| 4265/4266 | L302V/D364N/L376F/M383K/A386G/T408A/A451G/F484V/P598K | + |
| 4267/4268 | E643V | + |
| 4269/4270 | E643Y | + |
| 4271/4272 | S708R | + |
| 4273/4274 | Y464H | + |
| 4275/4276 | K711V | + |
| 4277/4278 | L245I/A423L | + |
| 4279/4280 | R572N | + |
| 4281/4282 | A800G | + |
| 4283/4284 | R352S/K363L | + |
| 4285/4286 | F281V | + |
| 4287/4288 | E643L | + |
| 4289/4290 | Q622G | + |
| 4291/4292 | P510S | + |
| 4293/4294 | A455G | + |
| 4295/4296 | S469R | + |
| 4297/4298 | E372K | + |
| 4299/4300 | T172V | + |
| 4301/4302 | A451G/P598K/T725H | + |
| 4303/4304 | E690G | + |
| 4305/4306 | E529S | + |
| 4307/4308 | E690T | + |
| 4309/4310 | L691F/G692H | + |
| 4311/4312 | K813G | + |
| 4313/4314 | P825Y | + |
| 4315/4316 | L389S | + |
| 4317/4318 | V368L | + |
| 4319/4320 | P311K | + |
| 4321/4322 | R489Q | + |
| 4323/4324 | K711S | + |
| 4325/4326 | P18H/S321T/F484V/L547M/T725H | + |
| 4327/4328 | K249E/T725H/A744G/L798F | + |
| 4329/4330 | N675M | + |
| 4331/4332 | H422R | + |
| 4333/4334 | L361C | + |
| 4335/4336 | E827G | + |
| 4337/4338 | L547M | + |
| 4339/4340 | A455L | + |
| 4341/4342 | P825G | + |
| 4343/4344 | L376W/Q699L | + |
| 4345/4346 | W715Q | + |
| 4347/4348 | G612S | + |
| 4349/4350 | L628M | + |
| 4351/4352 | Q703V | + |
| 4353/4354 | P18H/A423E/E529D/Y695K | + |
| 4355/4356 | A525G | + |
| 4357/4358 | R501A | + |
| 4359/4360 | A800M | + |
| 4361/4362 | P18H | + |
| 4363/4364 | L302S | + |
| 4365/4366 | V819L | + |
| 4367/4368 | L732E | + |
| 4369/4370 | E803S | + |
| 4371/4372 | L330M | + |
| 4373/4374 | Q622L | + |
| 4375/4376 | T172H | + |
| 4377/4378 | A314L | + |
| 4379/4380 | G353T | + |
| 4381/4382 | H348A | + |
| 4383/4384 | I623V | + |
| 4385/4386 | P559L | + |
| 4387/4388 | I555T/R572H | + |
| 4389/4390 | Y32L | + |
| 4391/4392 | E253I | + |
| 4393/4394 | G692S/A766T | + |
| 4395/4396 | Y728W | + |
| 4397/4398 | H422A | + |
| 4399/4400 | L361W | + |
| 4401/4402 | P18H/L291Q/Y695K | + |
| 4403/4404 | V462Q | + |
| 4405/4406 | L468A | + |
| 4407/4408 | L330V | + |
| 4409/4410 | K802R | + |
| 4411/4412 | I555V | + |
| 4413/4414 | A334T/G431C | + |
| 4415/4416 | A314M | + |
| 4417/4418 | V472G | + |
| 4419/4420 | P18H/L291Q/L302V/A684P | + |
| 4421/4422 | G353I | + |
| 4423/4424 | V819A | + |
| 4425/4426 | E372L | + |
| 4427/4428 | L590I/A652T | + |
| 4429/4430 | R521W | + |
| 4431/4432 | I555L | + |
| 4433/4434 | L468W | + |
| 4435/4436 | P559R | + |
| 4437/4438 | G431Q | + |
| 4439/4440 | Q703K | + |
| 4441/4442 | P347L | + |
| 4443/4444 | F484E | + |
| 4445/4446 | V462A | + |
| 4447/4448 | G431V | + |
| 4449/4450 | V472L | + |
| 4451/4452 | V472E | + |
| 4453/4454 | G692V | + |
| 4455/4456 | G431T | + |
| 4457/4458 | V368A | + |
| 4459/4460 | P18H/R139D/K249E/A423E/E433K/R501K/L547M/T725H | + |
| 4461/4462 | I555A | + |
| 4463/4464 | M655F | + |
| 4465/4466 | E643M | + |
| 4467/4468 | K540T | + |
| 4469/4470 | G600S | + |
| 4471/4472 | S392G | + |
| 4473/4474 | K363V | + |
| 4475/4476 | Y705F | + |
| 4477/4478 | A137L | + |
| 4479/4480 | G612V | + |
| 4481/4482 | V462G | + |
| 4483/4484 | G519D/L720S | + |
| 4485/4486 | P18H/P598K/T725H | + |
| 4487/4488 | A423E/L547M/P598K/Q642E | + |
| 4489/4490 | K64G | + |
| 4491/4492 | V368R | + |
| 4493/4494 | Q622S | + |
| 4495/4496 | N675G | + |
| 4497/4498 | R489H | + |
| 4499/4500 | A805M | + |
| 4501/4502 | Q703F | + |
| 4503/4504 | A455E | + |
| 4505/4506 | V462R | + |
| 4507/4508 | A577S | + |
| 4509/4510 | R492Y | + |
| 4511/4512 | A149P | + |
| 4513/4514 | P323W | + |
| 4515/4516 | Y728F | + |
| 4517/4518 | A455S | + |
| 4519/4520 | V472S | + |
| 4521/4522 | Q430R | + |
| 4523/4524 | A805Q | + |
| 4525/4526 | E533R | + |
| 4527/4528 | R358E | + |
| 4529/4530 | A753G | + |
| 4531/4532 | L389G | + |
| 4533/4534 | E533L | + |
| 4535/4536 | M756T | + |
| 4537/4538 | A753K | + |
| 4539/4540 | A766G | + |
| 4541/4542 | L354M | + |
| 4543/4544 | K802G | + |
| 4545/4546 | L550Y | + |
| 4547/4548 | L479W | + |
| 4549/4550 | V462L | + |
| 4551/4552 | G431S | + |
| 4553/4554 | K249E/A423E/L426R/T673K | + |
| 4555/4556 | M756R | + |
| 4557/4558 | A805F | + |
| 4559/4560 | A525R/E533C | + |
| 4561/4562 | R139D/K249E/L291Q/A451G/A744G | + |
| 4563/4564 | R414S | + |
| 4565/4566 | P588G | + |
| 4567/4568 | A752L | + |
| 4569/4570 | N574M | + |
| 4571/4572 | A149V | + |
| 4573/4574 | R160G | + |
| 4575/4576 | S321T/R448V/E529D/T673K | + |
| 4577/4578 | R532L | + |
| 4579/4580 | E533G | + |
| 4581/4582 | A525Q | + |
| 4583/4584 | L769M | + |
| 4585/4586 | R139D/K249E/L426R/A451G/R501K/T673K/A744G | + |
| 4587/4588 | P18H/S321T/A423E | + |
| 4589/4590 | A455V | + |
| 4591/4592 | P510T | + |
| 4593/4594 | A577L | + |
| 4595/4596 | W654K | + |
| 4597/4598 | Y464V | + |
| 4599/4600 | P311G | + |
| 4601/4602 | Y738G | + |
| 4603/4604 | E697Q | + |
| 4605/4606 | P825S | + |
| 4607/4608 | E827V | + |
| 4609/4610 | A474R | + |
| 4611/4612 | A577G | + |
| 4613/4614 | K540C | + |
| 4615/4616 | K711W | + |
| 4617/4618 | L769A | + |
| 4619/4620 | R557L | + |
| 4621/4622 | V462W | + |
| 4623/4624 | A474E | + |
| 4625/4626 | H422G | + |
| 4627/4628 | A423E/E433K/F484V/E529D/E533C/Q642E | + |
| 4629/4630 | F484G | + |
| 4631/4632 | R572T | + |
| 4633/4634 | P588V | + |
| 4635/4636 | A660R | + |
| 4637/4638 | P311H | + |
| 4639/4640 | L302V/A423E/E433K/E529D/Y695K | + |
| 4641/4642 | P588F | + |
| 4643/4644 | S392T | + |
| 4645/4646 | R256S/A455F | + |
| 4647/4648 | A455Q | + |
| 4649/4650 | A149G | + |
| 4651/4652 | P18H/L291Q/L302V/A423E/A684P | + |
| 4653/4654 | E480G | + |
| 4655/4656 | Y738M | + |
| 4657/4658 | A800P | + |
| 4659/4660 | W715V | + |
| 4661/4662 | A474S | + |
| 4663/4664 | Q498D | + |
| 4665/4666 | P18H/K249E/L302V/A423E/F484V/L547M/Y695K | + |
| 4667/4668 | P18H/A105F/S321T/L547M | + |
| 4669/4670 | P18H/L291Q/L302V/A525R/L547M/S659G/A684P/Y695K | + |
| 4671/4672 | L550G | + |
| 4673/4674 | M655A | + |
| 4675/4676 | W654F | + |
| 4677/4678 | I623W | + |
| 4679/4680 | Q601F | + |
| 4681/4682 | N675T | + |
| 4683/4684 | G600K | + |
| 4685/4686 | N743W | + |
| 4687/4688 | R271L | + |
| 4689/4690 | L720G | + |
| 4691/4692 | Q703G | + |
| 4693/4694 | S392V | + |
| 4695/4696 | L376I | + |
| 4697/4698 | E827W | + |
| 4699/4700 | V368W | + |
| 4701/4702 | A660G | + |
| 4703/4704 | P347R | + |
| 4705/4706 | L436F/N743K | + |
| 4707/4708 | L769V | + |
| 4709/4710 | E690W | + |
| 4711/4712 | R448V/A451G/E529D/T725H | + |
| 4713/4714 | N592S | + |
| 4715/4716 | F685R | + |
| 4717/4718 | Q575R | + |
| 4719/4720 | L38M/A463G | + |
| 4721/4722 | F484T | + |
| 4723/4724 | L618C | + |
| 4725/4726 | R726T | + |
| 4727/4728 | K802C | + |
| 4729/4730 | Q575C | + |
| 4731/4732 | A766V | + |
| 4733/4734 | H422L | + |
| 4735/4736 | A474L/P821S | + |
| 4737/4738 | P779R | + |
| 4739/4740 | E829L | + |
| 4741/4742 | T553S | + |
| 4743/4744 | P18H/L291Q/L302V/E433K | + |
| 4745/4746 | A455W | + |
| 4747/4748 | M655V | + |
| 4749/4750 | Q601L | + |
| 4751/4752 | E697G | + |
| 4753/4754 | E643G | + |
| 4755/4756 | H570S | + |
| 4757/4758 | L615Y | + |
| 4759/4760 | A577W | + |
| 4761/4762 | P18H/K249E/S299N/L302V/A423E/E529D/ | + |
| | E533C/Q642E/S659G/T673K | |
| 4763/4764 | L291Q/L302V/E529D | + |
| 4765/4766 | P18H/K249E/T673K | + |
| 4767/4768 | A411E | + |
| 4769/4770 | L302V/A423E/L547M/A684P/Y695K | + |
| 4771/4772 | N743Q | + |
| 4773/4774 | G692L | + |
| 4775/4776 | F484W | + |
| 4777/4778 | A577H | + |
| 4779/4780 | A800I | + |
| 4781/4782 | A805I | + |
| 4783/4784 | Y32G | + |
| 4785/4786 | E372W | + |
| 4787/4788 | P18H/L302V/A423E/E529D | + |
| 4789/4790 | E690R | + |
| 4791/4792 | S524K | + |
| 4793/4794 | E546R | + |
| 4795/4796 | K249E/S321T/T673K/L798F | + |
| 4797/4798 | L330T | + |
| 4799/4800 | P18H/L302V/A525R/E529D/L547M | + |
| 4801/4802 | T597S | + |
| 4803/4804 | M383R | + |
| 4805/4806 | R492K | + |
| 4807/4808 | A805T | + |
| 4809/4810 | P18H/S299N/A451G/A525R/E529D/Y695K | + |
| 4811/4812 | E730R | + |
| 4813/4814 | Y464K | + |
| 4815/4816 | L628T | + |
| 4817/4818 | A753L | + |
| 4819/4820 | G431D | + |
| 4821/4822 | G677A | + |
| 4823/4824 | R358L | + |
| 4825/4826 | K540S | + |
| 4827/4828 | L376W | + |
| 4829/4830 | P18H/E433K | + |
| 4831/4832 | P18H/E433K/T673K/T725H | + |
| 4833/4834 | T566G | + |
| 4835/4836 | E730V | + |
| 4837/4838 | Y620T | + |
| 4839/4840 | W654G | + |
| 4841/4842 | N743R | + |
| 4843/4844 | L291Q/A423E/E529D/E533C/P598K/Q642E/T725H/L798F | + |
| 4845/4846 | G612E | + |
| 4847/4848 | L550I | + |
| 4849/4850 | V462S | + |
| 4851/4852 | L468T | + |
| 4853/4854 | A455P/T518I | + |
| 4855/4856 | L361R | + |
| 4857/4858 | G600R | + |
| 4859/4860 | A525R | + |
| 4861/4862 | E433K/A525R/E529D/S659G/A684P/Y695K | + |
| 4863/4864 | R256V | + |
| 4865/4866 | P18H/R139D/P197K/K249E/S299N/L302V/ | + |
| | E433K/L547M/Q642E/S659G/Y695K/A744G | |
| 4867/4868 | A525V | + |
| 4869/4870 | A660E | + |
| 4871/4872 | T172W | + |
| 4873/4874 | P18H/L291Q/S659G | + |
| 4875/4876 | E533N | + |
| 4877/4878 | Y728A | + |
| 4879/4880 | Q703P | + |
| 4881/4882 | P18H/A105F/R139D/P197K/K249E/L426R/P598K | + |
| 4883/4884 | Y32S | + |
| 4885/4886 | T553L | + |
| 4887/4888 | F573T | + |
| 4889/4890 | P311C | + |
| 4891/4892 | R568K | + |
| 4893/4894 | E253C | + |
| 4895/4896 | A660W | + |
| 4897/4898 | F484Q | + |
| 4899/4900 | A423E/E433K/A487V/A525R/L547M | + |
| 4901/4902 | L583M | + |
| 4903/4904 | T571Y | + |
| 4905/4906 | L531A | + |
| 4907/4908 | L302M | + |
| 4909/4910 | R256F | + |
| 4911/4912 | P18H/A105F/R139D/P197K/L291Q/S299N/ | + |
| | A423E/L426R/R448V/E529D/L547M/T725H | |
| 4913/4914 | A752V | + |
| 4915/4916 | T597V | + |
| 4917/4918 | P18H/P197K/K249E/L291Q/F484V | + |
| 4919/4920 | P18H/L302V/E433K/L547M/A684P | + |
| 4921/4922 | L291Q/A525R | + |
| 4923/4924 | P510G | + |
| 4925/4926 | L376K | + |
| 4927/4928 | D505V | + |
| 4929/4930 | T597G | + |
| 4931/4932 | R160L | + |
| 4933/4934 | R568C | + |
| 4935/4936 | P18H/S299N/L302V/A423E/L426R/A525R/E529D | + |
| 4937/4938 | A463T | + |
| 4939/4940 | N743G | + |
| 4941/4942 | R501S | + |
| 4943/4944 | H422Q | + |
| 4945/4946 | Y728V | + |
| 4947/4948 | L583Q | + |
| 4949/4950 | Y738W | + |
| 4951/4952 | M383I | + |
| 4953/4954 | P18H/A105F/K249E/R284C/L302V/A423E/ | + |
| | L426R/R501K/T673K/A684P/L798F | |
| 4955/4956 | D664L | + |
| 4957/4958 | P18H/P197K/S321T/L426R/R448V/A451G/ | + |
| | S659G/T673K/A744G | |
| 4959/4960 | P18H/L302V/E529D/S659G/Y695K | + |
| 4961/4962 | L291Q/L302V/A423E | + |
| 4963/4964 | T576K | + |
| 4965/4966 | E372R | + |
| 4967/4968 | G612T | + |
| 4969/4970 | I623G | + |
| 4971/4972 | E253M | + |
| 4973/4974 | Y705G | + |
| 4975/4976 | M682K | + |
| 4977/4978 | L389R | + |
| 4979/4980 | Y728Q | + |
| 4981/4982 | F504S/A548F | + |
| 4983/4984 | H793T | + |
| 4985/4986 | A525S | + |
| 4987/4988 | L769C | + |
| 4989/4990 | Y705M | + |
| 4991/4992 | E829W | + |
| 4993/4994 | L354G | + |
| 4995/4996 | A105F/R139D/R448V/R501K/A525R/E529D/ | + |
| | Q642E/S659G | |
| 4997/4998 | F685S | + |
| 4999/5000 | T576W | + |
| 5001/5002 | Y728S | + |
| 5003/5004 | M383S | + |
| 5005/5006 | V15M/E167V | + |
| 5007/5008 | P18H/P197K/S321T/E433K/F484V | + |
| 5009/5010 | M756H | + |
| 5011/5012 | S392R | + |
| 5013/5014 | Y620L | + |
| 5015/5016 | Y705V | + |
| 5017/5018 | M655L | + |
| 5019/5020 | P197K/A525R/E529D/S659G/A744G | + |
| 5021/5022 | L302V/A684P/Y695K | + |
| 5023/5024 | P197K/L547M | + |
| 5025/5026 | L590F | + |
| 5027/5028 | K363T | + |
| 5029/5030 | L558Q | + |
| 5031/5032 | L468G | + |
| 5033/5034 | L302V/A423E/A525R/E529D | + |
| 5035/5036 | L291Q/A423E/A525R | + |
| 5037/5038 | L291Q/F484V/P598K/Q642E/T673K/A744G/L798F | + |
| 5039/5040 | P197K/A684P/T725H | + |
| 5041/5042 | L316N | + |
| 5043/5044 | P18H/R139D/P197K/L426R/P598K | + |
| 5045/5046 | P197K/L302V/P391S/L547M/P598K | + |
| 5047/5048 | E167G | + |
| 5049/5050 | A105F/P197K/K249E/A423E/L426R/L547M/Q642E/T673K | + |
| 5051/5052 | P197K/K249E/W327L/A423E/T673K/E690G | + |
| 5053/5054 | K363L | + |
| 5055/5056 | E829P | + |
| 5057/5058 | E167A | + |
| 5059/5060 | I623L | + |
| 5061/5062 | L769G | + |
| 5063/5064 | Y728R | + |
| 5065/5066 | L164V/T172F | + |
| 5067/5068 | Y620M | + |
| 5069/5070 | P18H/R139D/P197K/Q642E | + |
| 5071/5072 | L550P | + |

¹Levels of increased activity were determined relative to the reference polypeptide of SEQ ID NO: 3796, and defined as follows: “+” > 0.85.

### Example 31

### Improvements Over SEQ ID NO: 4182 in Enzyme Sensitivity

SEQ ID NO: 4182 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a RT-qPCR reaction, as described below.

Each variant was tested in a 15 μL reaction mixture comprising 5 copies of HeLa RNA fragment (Takara CAT #636543), primers Applied Biosystems Human GAPD (GAPDH) Endogenous Control (FAM™/MGB probe, non-primer limited) CAT #4352934E, 1× CODEX® HiTemp reaction buffer (Codexis), 1.25 ng/uL of CODEX® HiTemp reverse transcriptase (Codexis), and 2 vol % HTP lysate of each variant. RT-qPCR cycling (50° C. 5 min, 95° C. 2 min, 95° C. 3 sec, 60° C. 15 sec, for 50 cycles) was conducted in a CFX384 Touch Real-Time PCR Detection System (BioRad). For each variant, the Fold Improvement over Positive Control (FIOP) was calculated by the shift in Cq, the quantification cycle at which fluorescence level is higher than then threshold, relative to the Cq value of the positive control. The results are shown in Table 31.1.

TABLE 31.1
Activity Relative to SEQ ID NO: 4182

| SEQ ID | | FIOP HeLa RT-qPCR |
|---|---|---|
| NO: | Amino Acid Differences | (Relative to SEQ ID |
| (nt/aa) | (Relative to SEQ ID NO: 4182) | NO: 4182)¹ |
| 5073/5074 | P18H/A149G/K424N/F484V/R489L/N743A | + |
| 5075/5076 | P18H/A149G/P300G/E423A/K424N/N743A/L769C | + |
| 5077/5078 | P18H/E372S/K424N/E533W/E546C/G600A/T673K/L769C | + |
| 5079/5080 | R256S/P300G/K363L/E423A/K424N/E546C/T673K/E690V | + |
| 5081/5082 | K424N/G723D | + |
| 5083/5084 | P18H/R256S/E423A/K424N/A455S/T673K | + |
| 5085/5086 | A149G/R256S/P300G/E372S/K424N/E546C | + |
| 5087/5088 | A149G/K424N | + |
| 5089/5090 | K424N/E533W | + |
| 5091/5092 | P18H/K424N | + |
| 5093/5094 | K424N | + |
| 5095/5096 | A149G/R256S/P300G/L302G/K424N/F484V/L769C | + |

¹Levels of increased activity were determined relative to the reference polypeptide of SEQ ID NO: 4182, and defined as follows: “+” > 0.95.

### Example 32

### Improvements Over SEQ ID NO: 5094 in Enzyme Sensitivity and Activity

SEQ ID NO: 5094 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a qPCR reaction as described below.

For each variant tested, a 15 μL reaction mixture comprising 25 copies of SARS-CoV2 DNA fragment (CAT_10006625_2019-nCoV_N_Positive Control; IDT), 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture; ThermoFisher), 1× CODEX® HiTemp reaction buffer (Codexis), 2 mg/mL rHSA (Bovine serum albumin), 1 vol % HTP lysate of each variant. Then, qPCR cycling (95° C. 2 min, 95° C. 5 sec, 60° C. 5 sec, for 50 cycles) was conducted in a CFX384 Touch Real-Time PCR Detection System (BioRad). For each variant, the Fold Improvement over Positive Control (FIOP) was calculated by the shift in Cq where Cq is the quantification cycle at which fluorescence level is higher than then threshold, relative to the Cq value of the positive control. The results are shown in Table 32.1.

TABLE 32.1
Activity Relative to SEQ ID NO: 5094

| | | FIOP |
|---|---|---|
| | | Covid-19 |
| | | qPCR |
| SEQ | | (Relative |
| ID NO: | Amino Acid Differences | to SEQ ID |
| (nt/aa) | (Relative to SEQ ID NO: 5094) | NO: 5094)¹ |
| 5097/5098 | H630M | + |
| 5099/5100 | P511R | + |
| 5101/5102 | K517R | + |
| 5103/5104 | G359T | + |
| 5105/5106 | L45R/G268R/H475E/R489H | + |
| 5107/5108 | A744G | + |
| 5109/5110 | E817K | + |
| 5111/5112 | P598F/E611P/S708G | + |
| 5113/5114 | L417G | + |
| 5115/5116 | L436R | + |
| 5117/5118 | E611P | + |
| 5119/5120 | R621S/R639H/G748S/K749V | + |
| 5121/5122 | Y695P | + |
| 5123/5124 | K549L | + |
| 5125/5126 | A412G | + |
| 5127/5128 | L436R/S708G/A744G | + |
| 5129/5130 | M547L | + |
| 5131/5132 | A700G | + |
| 5133/5134 | E795S | + |
| 5135/5136 | R614A | + |
| 5137/5138 | E433A | + |
| 5139/5140 | S469H/H475E/R621S/R721E/G748S | + |
| 5141/5142 | S469L | + |
| 5143/5144 | D741H | + |
| 5145/5146 | T725P | + |
| 5147/5148 | G268R/H475E/M547L | + |
| 5149/5150 | L426R | + |
| 5151/5152 | T408W | + |
| 5153/5154 | G723A | + |
| 5155/5156 | L789D | + |
| 5157/5158 | Q791R | + |
| 5159/5160 | P598F | + |
| 5161/5162 | E611K | + |
| 5163/5164 | L385V | + |
| 5165/5166 | A714S | + |
| 5167/5168 | S469H/G748S | + |
| 5169/5170 | A306P | + |
| 5171/5172 | D741L | + |
| 5173/5174 | Y695L | + |
| 5175/5176 | A412P | + |
| 5177/5178 | E817A | + |
| 5179/5180 | S708G | + |
| 5181/5182 | A700R | + |
| 5183/5184 | R787W | + |
| 5185/5186 | A744S | + |
| 5187/5188 | S469H/H475E/K749V | + |
| 5189/5190 | L45R/G268R/H475E | + |
| 5191/5192 | D364G | + |
| 5193/5194 | V538S | + |
| 5195/5196 | E611S | + |
| 5197/5198 | V538A | + |
| 5199/5200 | G748S | + |
| 5201/5202 | N393E | + |
| 5203/5204 | L789T | + |
| 5205/5206 | L425T | + |
| 5207/5208 | E611L | + |
| 5209/5210 | L302P/K749V | + |
| 5211/5212 | E506H | + |
| 5213/5214 | A386S | + |
| 5215/5216 | S469H/R621S/G748S | + |
| 5217/5218 | G359T/A412G/E433A/E611P/R614A/A744S/L789D | + |
| 5219/5220 | R532W | + |
| 5221/5222 | H630A | + |
| 5223/5224 | L45R/G268R/L302P/S469H | + |
| 5225/5226 | G268R | + |
| 5227/5228 | Q699A | + |
| 5229/5230 | A617C | + |
| 5231/5232 | A386L | + |
| 5233/5234 | N467A/S469H | + |
| 5235/5236 | A487R | + |
| 5237/5238 | G339V | + |
| 5239/5240 | A838V | + |
| 5241/5242 | R787G | + |
| 5243/5244 | E817Q | + |
| 5245/5246 | L789Y | + |
| 5247/5248 | A487Y | + |
| 5249/5250 | V341M/R532L | + |
| 5251/5252 | E423A/N467A/R721E | + |
| 5253/5254 | L385P | + |
| 5255/5256 | V483A | + |
| 5257/5258 | G404P | + |
| 5259/5260 | N467R/S469H | + |
| 5261/5262 | L302P/R489H/M547L/R621S/Q699A/K749V | + |
| 5263/5264 | E397D | + |
| 5265/5266 | L45R/E423A/N467A/S469H/K749V | + |
| 5267/5268 | L45R/E423A/H475E/R557P/R621S/K749V | + |
| 5269/5270 | R489H | + |
| 5271/5272 | T408N | + |
| 5273/5274 | E423A/S469H | + |
| 5275/5276 | R557P | + |
| 5277/5278 | A412Q | + |
| 5279/5280 | K413W | + |
| 5281/5282 | N467M/S469H | + |
| 5283/5284 | K549H | + |
| 5285/5286 | D587M | + |
| 5287/5288 | A332D | + |
| 5289/5290 | D587V | + |
| 5291/5292 | A767T | + |
| 5293/5294 | E423A/N467A/M547L/R557P/R621S/Q699A/R721E | + |
| 5295/5296 | E423A/S469H/M547L/R557P/R621S/K749V | + |
| 5297/5298 | S584V | + |
| 5299/5300 | L507K | + |
| 5301/5302 | L45R/S469H/K749V | + |
| 5303/5304 | G748D | + |
| 5305/5306 | E397L | + |
| 5307/5308 | S469H/E471Q | + |
| 5309/5310 | L351M | + |
| 5311/5312 | E433W | + |
| 5313/5314 | Y439L | + |
| 5315/5316 | G268R/E423A | + |
| 5317/5318 | E397A | + |
| 5319/5320 | P821Q | + |
| 5321/5322 | T408P | + |
| 5323/5324 | N467A/R721E | + |
| 5325/5326 | L302P/R721E | + |
| 5327/5328 | R621S/K749V | + |
| 5329/5330 | R721E | + |
| 5331/5332 | S708T | + |
| 5333/5334 | R787P | + |
| 5335/5336 | K549Y | + |
| 5337/5338 | E533K/L542S | + |
| 5339/5340 | Y695A | + |
| 5341/5342 | P378C | + |
| 5343/5344 | S469H/E471L | + |
| 5345/5346 | K413H | + |
| 5347/5348 | L45R/G268R/R489H/R621S/K749V | + |
| 5349/5350 | V449G | + |
| 5351/5352 | D741E | + |
| 5353/5354 | E817D | + |
| 5355/5356 | R448W | + |
| 5357/5358 | L425S | + |
| 5359/5360 | L45R | + |
| 5361/5362 | A838R | + |
| 5363/5364 | L426V | + |
| 5365/5366 | A412T/Q689V | + |
| 5367/5368 | E817V | + |
| 5369/5370 | G268R/N467A/G748S | + |
| 5371/5372 | S469H/E471H | + |
| 5373/5374 | Q780A | + |
| 5375/5376 | A714Y | + |
| 5377/5378 | L45R/G268R/R621S/R721E | + |
| 5379/5380 | S469H/A473F | + |
| 5381/5382 | E817N | + |
| 5383/5384 | A306N | + |
| 5385/5386 | L45R/R721E | + |
| 5387/5388 | L45R/G268R/L302P/M547L/R557P/R621S/G748S/K749V | + |
| 5389/5390 | L691Q | + |
| 5391/5392 | R322D | + |
| 5393/5394 | L302P/E423A/R557P | + |
| 5395/5396 | P511V | + |
| 5397/5398 | A744P | + |
| 5399/5400 | E423A/S469H/H475E/R489H/R721E/G748S/K749V | + |
| 5401/5402 | L302P | + |
| 5403/5404 | A487L | + |
| 5405/5406 | E433S | + |
| 5407/5408 | R621H/R745D | + |
| 5409/5410 | K517V | + |
| 5411/5412 | E423A/R489H/R621S | + |
| 5413/5414 | L436T | + |
| 5415/5416 | Q780R | + |
| 5417/5418 | A744T | + |
| 5419/5420 | N467A/K749V | + |
| 5421/5422 | A412G/P598F/E611P/A744S | + |
| 5423/5424 | T408L | + |
| 5425/5426 | V483L | + |
| 5427/5428 | P598L | + |
| 5429/5430 | G404W | + |
| 5431/5432 | E611R | + |
| 5433/5434 | D741P | + |
| 5435/5436 | G359T/L436R/E611P/S708G/A744G | + |
| 5437/5438 | G785R/A809V | + |
| 5439/5440 | D539G | + |
| 5441/5442 | Q689W | + |
| 5443/5444 | K549V | + |
| 5445/5446 | Y695R | + |
| 5447/5448 | L45R/S469H/R721E | + |
| 5449/5450 | G359T/P598F/E611P/R614A/A744S | + |
| 5451/5452 | R322M | + |
| 5453/5454 | A700L | + |
| 5455/5456 | L385G | + |
| 5457/5458 | P598F/S708G | + |
| 5459/5460 | A306M | + |
| 5461/5462 | S469H/E471M | + |
| 5463/5464 | R322P | + |
| 5465/5466 | P378L | + |
| 5467/5468 | N467A/R621S | + |
| 5469/5470 | R448L | + |
| 5471/5472 | M547L/R621S/R721E/G748S/K749V | + |
| 5473/5474 | A335G | + |
| 5475/5476 | D741A | + |
| 5477/5478 | S469H/A473W | + |
| 5479/5480 | L45R/E423A/R489H/R557P | + |
| 5481/5482 | L45R/E423A/N467A/R621S | + |
| 5483/5484 | L45R/H475E/R489H/M547L/K749V | + |
| 5485/5486 | G359T/E611P/R614A/Y695L/A700G/A744G | + |
| 5487/5488 | E433Q | + |
| 5489/5490 | P378R | + |
| 5491/5492 | N424E | + |
| 5493/5494 | G359T/T408W/A412G/E611P/S708G/A744S | + |
| 5495/5496 | E329K/G359T/E611P/A700G/A744S | + |
| 5497/5498 | L45R/L302P | + |
| 5499/5500 | T408W/A744S | + |
| 5501/5502 | N393R | + |
| 5503/5504 | S321I | + |
| 5505/5506 | G9V/Q689G | + |
| 5507/5508 | A332V | + |
| 5509/5510 | D539A | + |
| 5511/5512 | P95S/G268R/L302P/E423A/R489H/M547L/R557P/ | + |
| | R721E/G748S/K749V | |
| 5513/5514 | E433A/S469L/E611P | + |
| 5515/5516 | G359T/A412G/P598F/E611K/A744S | + |
| 5517/5518 | A412G/S708G/A744G | + |
| 5519/5520 | T408A | + |
| 5521/5522 | A335C | + |
| 5523/5524 | E799Q | + |
| 5525/5526 | G268R/R489H/M547L | + |
| 5527/5528 | G359T/E433A/E611P/R614A/A700G | + |
| 5529/5530 | S708A | + |
| 5531/5532 | A386W | + |
| 5533/5534 | L45R/E423A/S469H/R489H/R621S | + |
| 5535/5536 | L45R/E423A/S469H/H475E/R621S/R721E/K749V | + |
| 5537/5538 | P598S | + |
| 5539/5540 | A344R | + |
| 5541/5542 | A386G | + |
| 5543/5544 | R745G | + |
| 5545/5546 | G359T/A412G/E433A/L436R/P598F/E611P/R614A | + |
| 5547/5548 | P598F/E611P | + |
| 5549/5550 | E611P/A744S | + |
| 5551/5552 | E433A/E611P/Y695L/A700G/A744G | + |
| 5553/5554 | R745D | + |
| 5555/5556 | L509V | + |
| 5557/5558 | A839V | + |
| 5559/5560 | L426G | + |
| 5561/5562 | P511L | + |
| 5563/5564 | A412G/E433A/P598F/E611P/A744G | + |
| 5565/5566 | L436R/E611K/R614A | + |
| 5567/5568 | A344P | + |
| 5569/5570 | A839P | + |
| 5571/5572 | P598R | + |
| 5573/5574 | A714V | + |
| 5575/5576 | G268R/N467A | + |
| 5577/5578 | L302P/N467A/R489H/G748S/K749V | + |
| 5579/5580 | N467C/S469H | + |
| 5581/5582 | T725V | + |
| 5583/5584 | V399L | + |
| 5585/5586 | L507Q | + |
| 5587/5588 | P511N | + |
| 5589/5590 | S469H/R621S | + |
| 5591/5592 | L45R/M547L/K749V | + |
| 5593/5594 | E817S | + |
| 5595/5596 | E611W | + |
| 5597/5598 | K413T | + |
| 5599/5600 | P511A | + |
| 5601/5602 | D364T | + |
| 5603/5604 | S469H/E471F | + |
| 5605/5606 | N467T/S469H | + |
| 5607/5608 | A332T | + |
| 5609/5610 | K602A | + |
| 5611/5612 | A306T/K459E | + |
| 5613/5614 | L789Q | + |
| 5615/5616 | K413L | + |
| 5617/5618 | D539G/T571A | + |
| 5619/5620 | K413E | + |
| 5621/5622 | S321A | + |
| 5623/5624 | R557P/R621S | + |
| 5625/5626 | G359T/E611K/R614A/A744G | + |
| 5627/5628 | A412G/Y695L/L789D | + |
| 5629/5630 | N424G | + |
| 5631/5632 | A652S | + |
| 5633/5634 | N424L | + |
| 5635/5636 | A344V | + |
| 5637/5638 | A617D | + |
| 5639/5640 | T408W/A412G/E433A/L436R/E611P/R614A/A744G | + |
| 5641/5642 | G359T/A412G/Y695L/S708G/A744G | + |
| 5643/5644 | V399Q | + |
| 5645/5646 | R532Y | + |
| 5647/5648 | L45R/E423A/N467A/H475E/M547L/R621S/R721E/K749V | + |
| 5649/5650 | L45R/G268R/E423A/S469H/R721E | + |
| 5651/5652 | L45R/G268R/H475E/R621S | + |
| 5653/5654 | Q791V | + |
| 5655/5656 | Y439S | + |
| 5657/5658 | N467A/M547L/R721E | + |
| 5659/5660 | L45R/G268R/L302P/S469H/M547L/R557P/R721E/K749V | + |
| 5661/5662 | L45R/G268R | + |
| 5663/5664 | R614A/A700G/S708G/A744S/L789D | + |
| 5665/5666 | A714R | + |
| 5667/5668 | T725S | + |
| 5669/5670 | R322A | + |
| 5671/5672 | E506V | + |
| 5673/5674 | P821K | + |
| 5675/5676 | A744R | + |
| 5677/5678 | L426T | + |
| 5679/5680 | G268R/E423A/S469H | + |
| 5681/5682 | G359T/S469L/E611K | + |
| 5683/5684 | G359T/P598F/A700G/A744G | + |
| 5685/5686 | S469H/E471A | + |
| 5687/5688 | D364R | + |
| 5689/5690 | L45R/G268R/L302P/N467A/R621S/G748S/K749V | + |
| 5691/5692 | L302P/N467A/S469H/M547L/R557P | + |
| 5693/5694 | G359T/P598F/R614A/Y695L/S708G/A744S | + |
| 5695/5696 | L789S | + |
| 5697/5698 | L777Q | + |
| 5699/5700 | L45R/G268R/S469H | + |
| 5701/5702 | A839E | + |
| 5703/5704 | P511D | + |
| 5705/5706 | L691W | + |
| 5707/5708 | R614T | + |
| 5709/5710 | E423A/S469H/M547L/R557P/K749V | + |
| 5711/5712 | R614A/L789D | + |
| 5713/5714 | A412G/L436R/A744G | + |
| 5715/5716 | L436R/E611P/R614A/Y695L | + |
| 5717/5718 | R614A/A744G | + |
| 5719/5720 | T408R | + |
| 5721/5722 | A838T | + |
| 5723/5724 | L385A | + |
| 5725/5726 | L296H/E433L | + |
| 5727/5728 | T408W/P598F/Y695L/A700G | + |
| 5729/5730 | E433A/L436R/P598F/A700G/A744G | + |
| 5731/5732 | A412G/R614A/S708G | + |
| 5733/5734 | G359T/A700G | + |
| 5735/5736 | A412G/S469L/E611P/Y695L/A744G | + |
| 5737/5738 | A412G/E433A/E611P/A744S | + |
| 5739/5740 | G359T/A412G/L436R/P598F/E611P/Y695L/A744G | + |
| 5741/5742 | H630G | + |
| 5743/5744 | L811S | + |
| 5745/5746 | S584Q | + |
| 5747/5748 | Y439A | + |
| 5749/5750 | G359T/A412G/S469L/E611P/Y695L/S708G/A744S | + |
| 5751/5752 | T408W/A412G/E611P/Y695L/S708G | + |
| 5753/5754 | G359T/A412G/P598F | + |
| 5755/5756 | L542S | + |
| 5757/5758 | A332L | + |
| 5759/5760 | A306S | + |
| 5761/5762 | V483T | + |
| 5763/5764 | E423A/R721E | + |
| 5765/5766 | G359T/T408W/A412G/A744S | + |
| 5767/5768 | G359T/T408W/E433A/R614A/S708G | + |
| 5769/5770 | Y695L/A744G | + |
| 5771/5772 | A306T | + |
| 5773/5774 | L425Y | + |
| 5775/5776 | S469Y | + |
| 5777/5778 | G373V | + |
| 5779/5780 | A839L | + |
| 5781/5782 | L302P/R621S | + |
| 5783/5784 | G748V | + |
| 5785/5786 | T725L | + |
| 5787/5788 | E817F | + |
| 5789/5790 | K549P | + |
| 5791/5792 | L777A | + |
| 5793/5794 | L45R/E423A/N467A | + |
| 5795/5796 | G359T/T408W/E611K/R614A/A744G | + |
| 5797/5798 | T408W/E611K/R614A/A744G | + |
| 5799/5800 | E611P/R614A/S708G/A744S/L789D | + |
| 5801/5802 | P821G | + |
| 5803/5804 | P598V | + |
| 5805/5806 | A332M | + |
| 5807/5808 | Y695S | + |
| 5809/5810 | L789G | + |
| 5811/5812 | A344G | + |
| 5813/5814 | K602V | + |
| 5815/5816 | E799M | + |
| 5817/5818 | Y439W | + |
| 5819/5820 | L385E | + |
| 5821/5822 | K413S | + |
| 5823/5824 | L45R/E423A/N467A/S469H/M547L/R721E/K749V | + |
| 5825/5826 | G359T/A412G/E433A/E611P/R614A/S708G | + |
| 5827/5828 | A412R | + |
| 5829/5830 | G373S | + |
| 5831/5832 | D741K | + |
| 5833/5834 | A617H | + |
| 5835/5836 | L45R/G268R/L302P/E423A | + |
| 5837/5838 | G359T/A412G/Y695L/A700G/A744G | + |
| 5839/5840 | T408W/A412G/L436R/E611P/R614A | + |
| 5841/5842 | A412G/S469L/E611P/A700G/A744S | + |
| 5843/5844 | R787K | + |
| 5845/5846 | L542P | + |
| 5847/5848 | K514M | + |
| 5849/5850 | L302P/E423A/H475E/R489H/M547L/R621S | + |
| 5851/5852 | T408W/A412G/P598F/A700G/A744S | + |
| 5853/5854 | Q689M | + |
| 5855/5856 | V538G | + |
| 5857/5858 | E611T | + |
| 5859/5860 | A700F | + |
| 5861/5862 | A386C | + |
| 5863/5864 | E397N | + |
| 5865/5866 | A335S | + |
| 5867/5868 | E397G | + |
| 5869/5870 | L45R/L302P/E423A/S469H/R621S | + |
| 5871/5872 | T408W/L436R/Y695L/S708G/A744G | + |
| 5873/5874 | T408W/E611P/A744S | + |
| 5875/5876 | E423G/P598F/A700G/S708G/A744S | + |
| 5877/5878 | L436G | + |
| 5879/5880 | L302P/E423A/S469H/R489H/R721E/G748S/K749V | + |
| 5881/5882 | L302P/E423A/N467A/R621S/G748S | + |
| 5883/5884 | G359T/T408W/A412G/E611K/R614A/A744S | + |
| 5885/5886 | T408W/A412G/S708G/A744G | + |
| 5887/5888 | T408W/A744G | + |
| 5889/5890 | P821S | + |
| 5891/5892 | A652G | + |
| 5893/5894 | A487S | + |
| 5895/5896 | E611C | + |
| 5897/5898 | G268R/H475E/R557P/R721E/K749V | + |
| 5899/5900 | A412G/L436R/E611K/R614A/A744S/L789D | + |
| 5901/5902 | T408W/P598F/E611P/R614A/A700G | + |
| 5903/5904 | G359T/T408W/A412G/A700G/A744G | + |
| 5905/5906 | L446S | + |
| 5907/5908 | K514G | + |
| 5909/5910 | G723T | + |
| 5911/5912 | K517A | + |
| 5913/5914 | Y695L/S708G/A744G | + |
| 5915/5916 | N424T | + |
| 5917/5918 | A332R | + |
| 5919/5920 | T725E | + |
| 5921/5922 | L425C | + |
| 5923/5924 | G404I | + |
| 5925/5926 | K602S/R724H | + |
| 5927/5928 | N393A | + |
| 5929/5930 | L302P/N467A/R621S | + |
| 5931/5932 | E423A/N467A/S469H | + |
| 5933/5934 | G359T/A744S | + |
| 5935/5936 | T408W/A700G | + |
| 5937/5938 | G359T/E611P/A700G/A744S | + |
| 5939/5940 | T725W | + |
| 5941/5942 | S469W | + |
| 5943/5944 | G268R/L302P/E423A/R557P/R721E | + |
| 5945/5946 | T408W/E611P/R614A/S708G/A744G | + |
| 5947/5948 | S469L/R614A/Y695L | + |
| 5949/5950 | P598F/E611P/R614A | + |
| 5951/5952 | K413R | + |
| 5953/5954 | S469R | + |
| 5955/5956 | R322L | + |
| 5957/5958 | E433R | + |
| 5959/5960 | A744W | + |
| 5961/5962 | L351V | + |
| 5963/5964 | E345G | + |
| 5965/5966 | L509M | + |
| 5967/5968 | E423A/N467A/R621S/R721E/K749V | + |
| 5969/5970 | G359T/A412G | + |
| 5971/5972 | T408W/L436R/P598F/E611P/S708G/A744S | + |
| 5973/5974 | A344S | + |
| 5975/5976 | L789A | + |
| 5977/5978 | G748R | + |
| 5979/5980 | R614W | + |
| 5981/5982 | Q689K | + |
| 5983/5984 | K549M | + |
| 5985/5986 | A700G/A744G | + |
| 5987/5988 | G359T/A412G/P598F/E611P/Y695L/A700G/S708G/A744S | + |
| 5989/5990 | E433A/L436R/P598F/A744G | + |
| 5991/5992 | A487V | + |
| 5993/5994 | L542K | + |
| 5995/5996 | R322T | + |
| 5997/5998 | L436Q | + |
| 5999/6000 | G339H | + |
| 6001/6002 | L436R/A700G/A744G | + |
| 6003/6004 | G359T/R614A | + |
| 6005/6006 | S469L/R614A/Y695L/A744S | + |
| 6007/6008 | A617L | + |
| 6009/6010 | A714G | + |
| 6011/6012 | K517M | + |
| 6013/6014 | S469L/A744S | + |
| 6015/6016 | S469H/E471R | + |
| 6017/6018 | R448E | + |
| 6019/6020 | T408W/E611P/R614A/Y695L/S708G | + |
| 6021/6022 | E611P/R614A | + |
| 6023/6024 | T408W/E433A/A700G/S708G/A744S | + |
| 6025/6026 | A714P | + |
| 6027/6028 | V399W | + |
| 6029/6030 | R485H/Q791S | + |
| 6031/6032 | D587S | + |
| 6033/6034 | E817H | + |
| 6035/6036 | L777C | + |
| 6037/6038 | L45R/E423A/S469H/H475E | + |
| 6039/6040 | A700V | + |
| 6041/6042 | E611V | + |
| 6043/6044 | Q689G | + |
| 6045/6046 | L417S | + |
| 6047/6048 | Q689R | + |
| 6049/6050 | P598F/R614A/A700G/A744G | + |
| 6051/6052 | L436A | + |
| 6053/6054 | K413G | + |
| 6055/6056 | L436S | + |
| 6057/6058 | A839R | + |
| 6059/6060 | E795D | + |
| 6061/6062 | R614G | + |
| 6063/6064 | R745V | + |
| 6065/6066 | A412G/E611K/R614A | + |
| 6067/6068 | E433A/L436R/E611P/R614A | + |
| 6069/6070 | A652V | + |
| 6071/6072 | K514T | + |
| 6073/6074 | A332I | + |
| 6075/6076 | S321W | + |
| 6077/6078 | K602W | + |
| 6079/6080 | L45R/L302P/H475E/R621S/G748S/K749V | + |
| 6081/6082 | E433A/A744S/L789D | + |
| 6083/6084 | A412G/E611P/R614A/S708G/A744S/L789D | + |
| 6085/6086 | G404F | + |
| 6087/6088 | R532T | + |
| 6089/6090 | L811R | + |
| 6091/6092 | Q791A | + |
| 6093/6094 | G359T/T408W/A412G | + |
| 6095/6096 | H83N/A700G/A744G | + |
| 6097/6098 | T408Q | + |
| 6099/6100 | Q689L | + |
| 6101/6102 | N393G | + |
| 6103/6104 | A617R | + |
| 6105/6106 | E611I | + |
| 6107/6108 | A487G | + |
| 6109/6110 | L45R/G268R/E423A/H475E/M547L/R721E/K749V | + |
| 6111/6112 | E423A/S469H/R721E | + |
| 6113/6114 | L302P/R557P/R721E | + |
| 6115/6116 | H630S | + |
| 6117/6118 | L542R | + |
| 6119/6120 | L798T | + |
| 6121/6122 | L436V | + |
| 6123/6124 | L436W | + |
| 6125/6126 | G404K | + |
| 6127/6128 | P378F | + |
| 6129/6130 | V399E | + |
| 6131/6132 | L789E | + |
| 6133/6134 | A700E | + |
| 6135/6136 | G268R/N467A/S469H/H475E | + |
| 6137/6138 | L45R/R557P | + |
| 6139/6140 | A306R | + |
| 6141/6142 | H630T | + |
| 6143/6144 | A386V | + |
| 6145/6146 | Y695E | + |
| 6147/6148 | A839T | + |
| 6149/6150 | G339A | + |
| 6151/6152 | T408W/P598F/E611P | + |
| 6153/6154 | T408W/A412G | + |
| 6155/6156 | A332Q | + |
| 6157/6158 | R322C | + |
| 6159/6160 | P598A | + |
| 6161/6162 | L542G | + |
| 6163/6164 | K549A | + |
| 6165/6166 | L542A | + |
| 6167/6168 | K549T | + |
| 6169/6170 | G359T/T408W/Y695L/A744G | + |
| 6171/6172 | G9D/L691S | + |
| 6173/6174 | K520V | + |
| 6175/6176 | G359T/T408W/Y695L/A700G/A744G | + |
| 6177/6178 | L302P/S469H/R489H/R621S/R721E/G748S | + |
| 6179/6180 | A306V | + |
| 6181/6182 | E409G | + |
| 6183/6184 | A617G | + |
| 6185/6186 | T408W/A412G/S469L/H475Q/E611P/Y695L/S708G/A744S | + |
| 6187/6188 | L691S | + |
| 6189/6190 | K549N | + |
| 6191/6192 | S584A | + |
| 6193/6194 | V538P | + |
| 6195/6196 | P598G | + |
| 6197/6198 | G723Q | + |
| 6199/6200 | Q791L | + |
| 6201/6202 | L417A | + |
| 6203/6204 | N467V/S469H | + |
| 6205/6206 | A617V | + |
| 6207/6208 | L45R/E423A | + |
| 6209/6210 | A306E | + |
| 6211/6212 | V399R | + |
| 6213/6214 | E345K | + |
| 6215/6216 | L509F | + |
| 6217/6218 | G373F | + |
| 6219/6220 | L811W | + |
| 6221/6222 | V399P | + |
| 6223/6224 | V483G | + |
| 6225/6226 | G373P | + |
| 6227/6228 | N467H/S469H | + |
| 6229/6230 | P821D | + |
| 6231/6232 | L45R/G268R/E423A/N467A/G748S | + |
| 6233/6234 | G748S/K749V | + |
| 6235/6236 | E506R | + |
| 6237/6238 | L302P/S469H | + |
| 6239/6240 | N467A/G748S | + |
| 6241/6242 | P378G | + |
| 6243/6244 | G785R | + |
| 6245/6246 | A700W | + |
| 6247/6248 | V399S | + |
| 6249/6250 | D364V | + |
| 6251/6252 | L302P/N467A/R721E/G748S | + |
| 6253/6254 | S708R | + |
| 6255/6256 | Y695T | + |
| 6257/6258 | A714L | + |
| 6259/6260 | G404T | + |
| 6261/6262 | A332C | + |
| 6263/6264 | P511Y | + |
| 6265/6266 | Q791H | + |
| 6267/6268 | K602S | + |
| 6269/6270 | A412L | + |
| 6271/6272 | N228D/S708E | + |
| 6273/6274 | R745P | + |
| 6275/6276 | L302P/E423A/M547L/R721E | + |
| 6277/6278 | E423A/S469H/R621S | + |
| 6279/6280 | S469H | + |
| 6281/6282 | R21S/Y695L/A700G | + |
| 6283/6284 | L417Q | + |
| 6285/6286 | H630W | + |
| 6287/6288 | N393Y | + |
| 6289/6290 | T408W/A412G/L436R/P598F/S708G | + |
| 6291/6292 | A412F | + |
| 6293/6294 | K602G | + |
| 6295/6296 | A744H | + |
| 6297/6298 | A767G | + |
| 6299/6300 | K514W | + |
| 6301/6302 | E423A/S469H/R489H/R557P/R621S/R721E/K749V | + |
| 6303/6304 | L45R/L302P/R621S | + |
| 6305/6306 | T408W/A412G/E433A/P598F/E611P/A700G/S708G/A744S | + |
| 6307/6308 | G339S | + |
| 6309/6310 | E817W | + |
| 6311/6312 | K517W | + |
| 6313/6314 | P821L | + |
| 6315/6316 | D364P | + |
| 6317/6318 | A386Q | + |
| 6319/6320 | L302P/R489H | + |
| 6321/6322 | T725C | + |
| 6323/6324 | R322G | + |
| 6325/6326 | D539L | + |
| 6327/6328 | L45R/N467A/R621S | + |
| 6329/6330 | E423A/N467A/R489H/R621S/R721E | + |
| 6331/6332 | V399G | + |
| 6333/6334 | E345L | + |
| 6335/6336 | G268R/E423A/N467A/M547L | + |
| 6337/6338 | G359T/L789D | + |
| 6339/6340 | G404L | + |
| 6341/6342 | G373K | + |
| 6343/6344 | E506G | + |
| 6345/6346 | G359T/T408W/P598F/A700G | + |
| 6347/6348 | R745W | + |
| 6349/6350 | G339L | + |
| 6351/6352 | E423A/G748S | + |
| 6353/6354 | G268R/L302P/R721E | + |
| 6355/6356 | Q791T | + |
| 6357/6358 | L542W | + |
| 6359/6360 | L45R/L302P/E423A/S469H/R621S/R721E/G748S/K749V | + |
| 6361/6362 | A412G/E611P/L789D | + |
| 6363/6364 | R322S | + |
| 6365/6366 | K517L | + |
| 6367/6368 | T408W/A412G/P598F/E611P/R614A/S708G | + |
| 6369/6370 | R721E/K749V | + |
| 6371/6372 | V483C | + |
| 6373/6374 | L691G | + |
| 6375/6376 | V483S | + |
| 6377/6378 | E506M | + |
| 6379/6380 | G785Q | + |
| 6381/6382 | A617S | + |
| 6383/6384 | V538L | + |
| 6385/6386 | G339P | + |
| 6387/6388 | R621S | + |
| 6389/6390 | L426S | + |
| 6391/6392 | R745Q | + |
| 6393/6394 | G268R/E423A/S469H/H475E | + |
| 6395/6396 | R787E | + |
| 6397/6398 | Y554S | + |
| 6399/6400 | K549S | + |
| 6401/6402 | L45R/L302P/E423A/S469H/H475E | + |
| 6403/6404 | E433G | + |
| 6405/6406 | A314S/V538L | + |
| 6407/6408 | L777V | + |
| 6409/6410 | A700Y | + |
| 6411/6412 | L385R | + |
| 6413/6414 | N393P | + |
| 6415/6416 | E423A | + |
| 6417/6418 | L45R/N467A/G748S | + |
| 6419/6420 | G723V | + |
| 6421/6422 | N467A/R489H/M547L/R621S/G748S | + |
| 6423/6424 | S469E | + |
| 6425/6426 | V483Q | + |
| 6427/6428 | L354I/G359T/T408W/P598F | + |
| 6429/6430 | L789P | + |
| 6431/6432 | L45R/G268R/L302P/H475E/R489H | + |
| 6433/6434 | L691A | + |
| 6435/6436 | G404S | + |
| 6437/6438 | Q791G | + |
| 6439/6440 | E423A/R489H | + |
| 6441/6442 | N467A/R721E/G748S | + |
| 6443/6444 | A412K | + |
| 6445/6446 | Y439E | + |
| 6447/6448 | D587G | + |
| 6449/6450 | L777T | + |
| 6451/6452 | Q689S | + |
| 6453/6454 | R621S/G748S | + |
| 6455/6456 | T580K | + |
| 6457/6458 | L691R | + |
| 6459/6460 | E443C | + |
| 6461/6462 | L45R/E423A/H475E/M547L/R721E | + |
| 6463/6464 | H475E | + |
| 6465/6466 | E546G/Q699A | + |
| 6467/6468 | A306L | + |
| 6469/6470 | L45R/N467A/S469H/R557P | + |
| 6471/6472 | L45R/E423A/R489H | + |
| 6473/6474 | D587T | + |
| 6475/6476 | E397P | + |
| 6477/6478 | H630F | + |
| 6479/6480 | A700G/A767T | + |
| 6481/6482 | N467W/S469H | + |
| 6483/6484 | A744F | + |
| 6485/6486 | K517S | + |
| 6487/6488 | L789V | + |
| 6489/6490 | R614P | + |
| 6491/6492 | L691T | + |
| 6493/6494 | K520S | + |
| 6495/6496 | E443T | + |
| 6497/6498 | L45R/L302P/S469H/M547L/R721E | + |
| 6499/6500 | E397V | + |
| 6501/6502 | D587W | + |
| 6503/6504 | L811G | + |
| 6505/6506 | G359T/Y695L/A700G/L789D | + |
| 6507/6508 | H630C | + |
| 6509/6510 | P598C | + |
| 6511/6512 | L507G | + |
| 6513/6514 | E397R | + |
| 6515/6516 | L116P | + |
| 6517/6518 | R596W | + |
| 6519/6520 | Q689P | + |
| 6521/6522 | L691K | + |
| 6523/6524 | L45R/L302P/E423A/M547L/R621S/R721E | + |
| 6525/6526 | P378Y | + |
| 6527/6528 | Y387C/H630E | + |
| 6529/6530 | G359T/S469L/E611P/A700G | + |
| 6531/6532 | V449W | + |
| 6533/6534 | R745C | + |
| 6535/6536 | W836L | + |
| 6537/6538 | G359T/T408W/A412G/E611K/A744S/L789D | + |
| 6539/6540 | G359T/S469L/E611P/R614A | + |
| 6541/6542 | G359T/P598F/A744S/L789D | + |
| 6543/6544 | G359T/E433A/E611P/Y695L/A700G/L789D | + |

¹Levels of increased activity were determined relative to the reference polypeptide of SEQ ID NO: 5094, and defined as follows: “+” > 0.85.

### Example 33

### Improvements Over SEQ ID NO: 5094 in Enzyme Sensitivity and Processivity (Activity) and Inhibitor Tolerance

SEQ ID NO: 5094 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a qPCR reaction as described below.

For each variant tested, a 15 μL reaction mixture comprising 20 copies of SARS-CoV2 DNA fragment (CAT_10006625_2019-nCoV_N_Positive Control; IDT), 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture; ThermoFisher), 1× CODEX® HiTemp reaction buffer (Codexis) 2 mg/mL rHSA (Bovine serum albumin), 1 vol % HTP lysate of each variant. The reaction also comprised either a single inhibitor or a combination of two inhibitors selected from 3% ethanol, 25 mM GITC, 10 μM hematin, 3.125 U/mL heparin, 0.01% SDS, 300 mM urea, 175 mM NaCl, and 250 ng gDNA. Then, qPCR cycling (95° C. 2 min, 95° C. 5 sec, 60° C. 5 sec, for 50 cycles) was conducted in a CFX384 Touch Real-Time PCR Detection System (BioRad). For each variant, the Fold Improvement over Positive Control (FIOP) was calculated by calculating the shift in Cq where Cq is the quantification cycle at which fluorescence level is higher than then threshold, relative to the Cq value of the positive control. The results are shown in Table 33.

TABLE 33.1
Activity Relative to SEQ ID NO: 5094

| SEQ ID | | FIOP Covid-19 qPCR |
|---|---|---|
| NO: | Amino Acid Differences | With Inhibitors (Relative |
| (nt/aa) | (Relative to SEQ ID NO: 5094) | to SEQ ID NO: 5094)¹ |
| 5189/5190 | L45R/G268R/H475E | + |
| 5105/5106 | L45R/G268R/H475E/R489H | + |
| 5377/5378 | L45R/G268R/R621S/R721E | + |
| 5385/5386 | L45R/R721E | + |
| 5261/5262 | L302P/R489H/M547L/R621S/Q699A/K749V | + |
| 5173/5174 | Y695L | + |
| 5223/5224 | L45R/G268R/L302P/S469H | + |
| 5225/5226 | G268R | + |
| 5325/5326 | L302P/R721E | + |
| 5143/5144 | D741H | + |
| 5167/5168 | S469H/G748S | + |
| 5103/5104 | G359T | + |
| 5147/5148 | G268R/H475E/M547L | + |
| 5155/5156 | L789D | + |
| 5125/5126 | A412G | + |
| 5129/5130 | M547L | + |
| 5159/5160 | P598F | + |
| 5177/5178 | E817A | + |
| 5141/5142 | S469L | + |
| 5137/5138 | E433A | + |
| 5209/5210 | L302P/K749V | + |
| 5187/5188 | S469H/H475E/K749V | + |
| 5165/5166 | A714S | + |
| 5115/5116 | L436R | + |
| 5265/5266 | L45R/E423A/N467A/S469H/K749V | + |
| 5117/5118 | E611P | + |
| 5175/5176 | A412P | + |
| 5135/5136 | R614A | + |
| 5163/5164 | L385V | + |
| 5131/5132 | A700G | + |
| 5149/5150 | L426R | + |
| 5233/5234 | N467A/S469H | + |
| 5251/5252 | E423A/N467A/R721E | + |
| 5181/5182 | A700R | + |
| 5139/5140 | S469H/H475E/R621S/R721E/G748S | + |
| 5107/5108 | A744G | + |
| 5179/5180 | S708G | + |
| 5269/5270 | R489H | + |
| 5151/5152 | T408W | + |
| 5185/5186 | A744S | + |
| 5421/5422 | A412G/P598F/E611P/A744S | + |
| 5217/5218 | G359T/A412G/E433A/E611P/R614A/A744S/L789D | + |
| 5401/5402 | L302P | + |
| 5161/5162 | E611K | + |
| 5199/5200 | G748S | + |
| 5329/5330 | R721E | + |
| 5227/5228 | Q699A | + |
| 5113/5114 | L417G | + |
| 5097/5098 | H630M | + |
| 5169/5170 | A306P | + |
| 5171/5172 | D741L | + |
| 5183/5184 | R787W | + |
| 5101/5102 | K517R | + |
| 5099/5100 | P511R | + |
| 5157/5158 | Q791R | + |
| 5435/5436 | G359T/L436R/E611P/S708G/A744G | + |
| 5111/5112 | P598F/E611P/S708G | + |
| 5215/5216 | S469H/R621S/G748S | + |
| 5119/5120 | R621S/R639H/G748S/K749V | + |
| 5109/5110 | E817K | + |
| 5275/5276 | R557P | + |
| 5127/5128 | L436R/S708G/A744G | + |
| 5133/5134 | E795S | + |
| 5123/5124 | K549L | + |
| 5121/5122 | Y695P | + |
| 5145/5146 | T725P | + |

¹Levels of increased activity were determined relative to the reference polypeptide of SEQ ID NO: 5094, and defined as follows: “+” > 0.9.

### Example 34

### Improvements Over SEQ ID NO: 5136 in Enzyme Sensitivity

SEQ ID NO: 5136 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a qPCR reaction as described below.

For each variant tested, a 15 μL reaction mixture comprising 20 copies of SARS-CoV2 DNA fragment (CAT_10006625_2019-nCoV_N_Positive Control; IDT), 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture; ThermoFisher), 1× CODEX® HiTemp reaction buffer (Codexis), 2 mg/mL rHSA (Bovine serum albumin) eEnzyme's cat no HSA-1rcl, 1 vol % HTP lysate of each variant. Then, qPCR cycling (95° C. 2 min, 95° C. 5 sec, 60° C. 5 sec, for 50 cycles) was conducted in a CFX384 Touch Real-Time PCR Detection System (BioRad). For each variant, the Fold Improvement over Positive Control (FIOP) was calculated as the average shift in Cq product yield formed by the variant relative to the control. Cq is the quantification cycle at which fluorescence level is higher than then threshold. The results are shown in Table 34.1.

TABLE 34.1
Activity Relative to SEQ ID NO: 5136

| | | FIOP Covid-19 |
|---|---|---|
| | | qPCR (Relative |
| SEQ ID | Amino Acid Differences | to SEQ ID |
| NO: (nt/aa) | (Relative to SEQ ID NO: 5136) | NO: 5136)¹ |
| 6609/6610 | L436R/E611P | + |
| 6547/6548 | T408W/A412G/L436R/A614R/A700G/A744S | + |
| 6643/6644 | E433A/A614R/A700G/A744S | + |
| 6625/6626 | T408W/E611P | + |
| 6553/6554 | A412G | + |
| 6563/6564 | A700G | + |
| 6545/6546 | L436R/P598F | + |
| 6619/6620 | G359T/P598F/Y695L | + |
| 6645/6646 | L436R/E611P/A614R/Y695L/A700G/A744S | + |
| 6565/6566 | G359T/A744S | + |
| 6581/6582 | L436R/P598F/A614R/Y695L/A744G | + |
| 6561/6562 | G359T/T408W/A614R | + |
| 6593/6594 | A700G/A744S | + |
| 6585/6586 | T408W/A412G/E611P | + |
| 6573/6574 | E433A/L436R/E611P/A614R/A744S | + |
| 6621/6622 | L436R/A744S | + |
| 6587/6588 | G359T/A412G/E611K/S708G/A744G | + |
| 6549/6550 | G359T/P598F/E611K | + |
| 6571/6572 | T408W/E433A/L436R/E611P/A614R/A744G | + |
| 6591/6592 | T408W/A412G/A744G | + |
| 6557/6558 | L436R/E611P/A614R | + |
| 6579/6580 | G359T/P598F/E611K/Y695L/S708G | + |
| 6603/6604 | G359T/E433A/A744G | + |
| 6551/6552 | G359T/E611K/A700G/S708G | + |
| 6583/6584 | E433A/L436R/P598F/E611P/A614R/S708G/A744S | + |
| 6629/6630 | E433A/L436R/A744G | + |
| 6567/6568 | A412G/E611K/A614R/S708G/A744S/L789D | + |
| 6627/6628 | E611P | + |
| 6595/6596 | G359T/A412G/P598F/E611P/A614R/A700G/S708G/A744S | + |
| 6623/6624 | A412G/A700G/A744S | + |
| 6605/6606 | E611P/S708G/A744S | + |
| 6613/6614 | E611P/A614R | + |
| 6569/6570 | A412G/E433A | + |
| 6597/6598 | A744S/L789D | + |
| 6637/6638 | L436R/E611P/S708G | + |
| 6611/6612 | A614R/A744G | + |
| 6555/6556 | T408W | + |
| 6615/6616 | T408W/E433A/L436R/Y695L/S708G/A744S | + |
| 6577/6578 | A614R/L789D | + |
| 6589/6590 | E433A/L436R/E611P/A744G | + |
| 6575/6576 | Y695L/A700G/A744G | + |
| 6617/6618 | G359T/L436R/P598F | + |
| 6631/6632 | G359T/A412G/L436R/P598F/E611K/A614R/Y695L/A700G/S708G/A744S | + |
| 6639/6640 | P598F/A614R/Y695L/A700G/S708G/A744S | + |
| 6641/6642 | Y695L | + |
| 6599/6600 | E611P/A614R/Y695L/A700G/S708G/A744S | + |
| 6635/6636 | P598F/E611P/A614R/S708G/A744G | + |
| 6559/6560 | E433A/S708G | + |
| 6607/6608 | G359T/S469L/A700G/A744G | + |
| 6633/6634 | G359T/L789D | + |
| 6601/6602 | T408W/A412G/S469L | + |

¹Levels of increased activity were determined relative to the reference polypeptide of SEQ ID NO: 5136, and defined as follows: “+” > 0.85.

### Example 35

### Improvements Over SEQ ID NO: 5136 in Enzyme Sensitivity, Activity and Inhibitor Tolerance

SEQ ID NO: 5136 was selected as the parent enzyme for the round of evolution and enzyme characterization described in this Example. Libraries of engineered genes were produced using techniques such as saturation mutagenesis and recombination of previously identified beneficial mutations. The polypeptides encoded by each gene were produced in HTP as described in Example 2, and the soluble lysate was generated as described in Example 3. Each variant was screened in a qPCR reaction as described below.

For each variant tested, a 15 μL reaction mixture comprising 20 copies of SARS-CoV2 DNA fragment (CAT_10006625_2019-nCoV_N_Positive Control; IDT), 500 nM N1 primers (SEQ ID NOS: 3262 and 3263), 125 nM FAM labeled probe (SEQ ID NO: 3264) (CDC EUA assay, Catalog #2019-nCoVEUA-01; IDT) Twist CAT #102019, 0.2 mM dNTPs (10 mM mixture; ThermoFisher), 1× CODEX® HiTemp reaction buffer (Codexis), 2 mg/mL rHSA (Bovine serum albumin) eEnzyme's cat no HSA-1rcl, 1 vol % HTP lysate of each variant. The reaction also comprised either a single inhibitor or a combination of two inhibitors selected from 3% ethanol, 25 mM GITC, 10 μM hematin, 6 U/mL heparin, 0.01% SDS, 300 mM urea, 75 mM KCl, and 250 ng gDNA. Then, qPCR cycling (95° C. 2 min, 95° C. 5 sec, 60° C. 10 sec, for 50 cycles) was conducted in a CFX384 Touch Real-Time PCR Detection System (BioRad). For each variant, the Fold Improvement over Positive Control (FIOP) was calculated as the average shift in Cq product yield formed by the variant relative to the control. Cq is the quantification cycle at which fluorescence level is higher than then threshold. The results are shown in Table 35.

TABLE 35.1
Activity Relative to SEQ ID NO: 5136

| | | FIOP Covid-19 qPCR With |
|---|---|---|
| SEQ ID | Amino Acid Differences | Inhibitors |
| NO: (nt/aa) | (Relative to SEQ ID NO: 5136) | (Relative to SEQ ID NO: 5136)¹ |
| 6609/6610 | L436R/E611P | + |
| 6563/6564 | A700G | + |
| 6619/6620 | G359T/P598F/Y695L | + |
| 6545/6546 | L436R/P598F | + |
| 6645/6646 | L436R/E611P/A614R/Y695L/A700G/A744S | + |
| 6553/6554 | A412G | + |
| 6643/6644 | E433A/A614R/A700G/A744S | + |
| 6547/6548 | T408W/A412G/L436R/A614R/A700G/A744S | + |
| 6625/6626 | T408W/E611P | + |

¹Levels of increased activity were determined relative to the reference polypeptide of SEQ ID NO: 5136, and defined as follows: “+” > 0.95.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes in the United States of America, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

What is claimed is:

1. An engineered DNA polymerase or a functional fragment thereof, comprising a polypeptide sequence having at least 75% or more sequence identity to a reference sequence comprising residues 12 to 841 of SEQ ID NO: 2, or to a reference sequence of SEQ ID NO: 2, wherein the polypeptide sequence comprises one or more substitutions relative to the reference sequence comprising residues 12 to 841 of SEQ ID NO: 2, or to the reference sequence of SEQ ID NO: 2.

2. The engineered DNA polymerase of claim 1, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2 or to the reference sequence of SEQ ID NO: 2.

3. The engineered DNA polymerase of claim 1, comprising one or more substitutions or substitution sets selected from 11/287, 15/633, 18/145/255, 20/602, 29, 50/255/557/685, 110, 113/170/194/235/249/319/645/659/660/674/699, 130, 145, 145/255/287/391/472/476/557, 145/255/287/391/476/513, 145/255/287/391/557/685/743/758, 145/255/391/398/472/476/513/557/602/660, 145/255/391/398/472/557/685, 145/255/391/398/476/557/758, 145/255/391/398/513/658/685/743, 145/255/391/472/476/685/743, 145/255/391/472/557/685, 145/255/391/476/557/602/660/743, 145/255/391/476/557/658/660/743, 145/255/391/476/758, 145/255/391/513/557/743/758, 145/255/391/557/758, 145/255/398/472/476/513/557/658/660, 145/255/472/476/557/685/743/758, 145/255/472/513/557/602/660/743, 145/255/513, 145/255/513/602, 145/255/557, 145/255/557/658/660/685, 145/255/743/758, 145/267/391/398/513/557/658/685/743, 145/287/391/472/557/602/658/660/685, 145/287/391/472/602/658, 145/287/391/476/513/557/602, 145/287/391/476/658/660/685, 145/287/658/758, 145/391, 145/391/398/476/758, 145/391/472/476/513/557/658/685/758, 145/391/472/476/557/602/660/685/743, 145/391/472/476/557/743/758, 145/391/472/476/658/660/758, 145/391/472/476/658/685/758, 145/391/472/476/758, 145/391/513/557/602, 145/391/513/557/658/685, 145/391/513/658/743/758, 145/391/513/758, 145/391/557, 145/391/557/658/758, 145/391/557/660/685, 145/391/557/743, 145/398/472/557/660, 145/476/557/660/685/743/758, 145/513/685, 145/557/658, 145/557/685/758, 145/685, 161/162, 161/162/170/194/235/423/699, 161/162/170/235/249/252, 161/162/194/269/605/660, 161/162/269/476/660, 161/162/352/434/605, 161/162/659/660/674, 161/170/235/249/252/434, 161/170/235/269/319, 161/170/235/319/412, 161/170/252/352/412/434/699, 161/170/476, 161/194/198/235/252/423/434/605/645/699, 161/194/235/249/352/412/434/674/699, 161/194/252, 161/194/252/323/352/412/605/659/660/674/699, 161/194/252/412/605, 161/194/434, 161/235/249/319/412/605/659, 161/235/252/319/352/645/674/699, 161/235/352/423/645/674, 161/235/645/699, 161/249/412/476/605/645/659/660/674, 161/252/319/352/412/423/476/605/645, 161/252/319/674, 161/269/434/699, 161/319/660, 161/352, 161/423/645, 161/605, 162, 162/170/194/235/249/269/674/699, 162/170/412/434/476/605, 162/194/249/645/659, 162/235/269/423/476/605/645/659, 162/252/352/412/434/645/660/699, 162/269/352/412/476/605, 162/352, 162/423/645, 162/605/645, 164, 168, 170, 170/194/198/235/674/699, 170/194/235/252/269/323/412/423/476/605/645/660/674, 170/194/235/319/412/476/699, 170/194/269/319/412/423/476/605/645, 170/194/319/352/476/699, 170/235/249/352/645, 170/235/269/352, 170/235/269/434/674, 170/235/319/352/412/434/476/645/660/674, 170/235/352/674/699, 170/235/352/699, 170/235/412, 170/235/645, 170/235/660, 170/249/252/269/352/412/423/434/476/659, 170/249/252/269/423/659, 170/249/412, 170/252/352/423/674/699, 170/269/352/423/476/605/659/660/699, 170/269/476/645, 170/412/423/476/605/645/660/674, 170/476/674, 175, 177, 194, 194/198/235/252/323/412/605/645, 194/198/252/319/423/645/674, 194/235/252, 194/235/252/319/423, 194/235/605, 194/235/645, 194/235/699, 194/249/252/645/699, 194/249/412/476/605/645/699, 194/252/269/319/434/605/645/659/674/699, 194/252/319/412/605, 194/269/319/412/605/674/699, 194/319/352/605/660, 194/323/699, 194/352/476/659, 194/352/605/645/699, 194/434, 194/645/659, 197, 198/674/699, 202/268, 217, 227, 235, 235/249/252/323/352/476, 235/249/252/412/605/645/699, 235/249/252/423/699, 235/249/252/434/605/645/699, 235/252, 235/252/269/352/434/605/674/699, 235/252/269/352/476/645/674, 235/252/269/423/605/645/660/674, 235/269/323/412/434/659/674/699, 235/269/674/699, 235/319/605/645/674, 235/352, 235/352/412/659/660/674/699, 235/352/605, 235/412/645, 235/476/674, 235/605/645, 249/252/319/352/645, 249/252/319/605/660, 249/252/412/423/645/674/699, 249/412/476/605, 252/352/645/660, 252/645, 253, 255/260/398/557, 255/287, 255/287/391/472/557/658/660, 255/287/391/472/658/660/685, 255/287/391/476/557, 255/287/391/658, 255/391, 255/391/398/476/557, 255/391/398/476/602/658/660/685, 255/391/398/513/557/658/743/758, 255/391/472/513/758, 255/391/472/557/658/660/685/743/758, 255/391/472/557/658/660/758, 255/391/472/658/660/758, 255/391/472/685, 255/391/476/513/557/602/685/758, 255/391/476/557/602/660/758, 255/391/476/557/658, 255/391/476/758, 255/391/513/602/658/660, 255/391/557, 255/391/602, 255/391/685/758, 255/391/743, 255/398/472/476/557/658, 255/398/476, 255/398/476/557/658/660/685, 255/398/513/758, 255/472/758, 255/513/557, 255/513/557/685/758, 255/557/658, 255/602, 255/660, 267, 268, 269/352, 269/352/412, 269/605, 270, 271/472, 279, 281, 287/391/472, 287/391/472/476/557/602/658/660/685/743, 287/391/476/513, 287/391/513/557/660/685/743/758, 287/391/557, 287/391/557/602/658/743, 287/391/557/685, 287/391/602, 287/685, 297, 319, 319/352/412/434/699, 319/434/476, 322, 329, 331, 339, 347, 350, 352, 352/412/605/645/699, 352/605/645/699, 352/605/659, 352/645, 354, 356, 358, 360, 361, 365, 372, 377, 379, 391, 391/398/476/685, 391/398/513/602/658/660/758, 391/451/472/513/557, 391/472/476/557/602, 391/472/513/557/602/658, 391/472/513/602/658, 391/476/513/743, 391/476/557, 391/513/658/660, 391/557, 391/557/758, 391/602, 391/602/758, 391/658, 391/660/743/758, 391/685, 396, 398, 398/472, 398/557/602/658, 412, 412/645, 412/674, 423, 423/476/605, 423/476/645/674, 423/645, 423/699, 425, 426, 435, 440, 455, 456, 467, 472, 472/476/513/743, 472/476/557/658/758, 472/513/557/658/743/758, 472/513/557/660/685, 472/513/658/660/743, 472/557/660/685/743/758, 472/758, 476, 476/557/758, 476/685, 476/758, 478/557, 479, 485, 506, 508, 513, 513/557/743, 513/557/758, 532, 533, 540, 557, 557/658/660, 557/658/685, 557/743, 564, 602, 602/685, 602/703, 605/699, 635, 643, 645, 645/659, 645/674/699, 658, 658/660, 658/685/758, 659, 660, 674, 704, 713, 718, 758, and 810, or combinations thereof, wherein the amino acid positions are relative to the reference sequence of SEQ ID NO: 2.

4. The engineered DNA polymerase of claim 1, comprising at least one or more substitutions or substitution sets at amino acid positions selected from 11/287, 29, 50/255/557/685, 145/255/287/391/472/476/557, 145/255/391/472/557/685, 145/255/391/685, 145/255/472/476/557/685/743/758, 145/255/557/658/660/685, 145/391/472/476/513/557/658/685/758, 145/391/513/557/602, 145/391/557/743, 145/398/472/557/660, 145/557/658, 145/743, 161/162/269/476/660, 161/162/659/660/674, 161/170/235/269/319, 161/170/235/319/412, 161/194/235/249/352/412/434/674/699, 161/194/252/412/605, 161/235/252/319/352/645/674/699, 161/235/645/699, 161/249/412/476/605/645/659/660/674, 161/252/319/352/412/423/476/605/645, 161/319/660, 161/352, 162, 162/352, 162/423/645, 170, 170/194/235/252/269/323/412/423/476/605/645/660/674, 170/235/249/352/645, 170/235/319/352/412/434/476/645/660/674, 170/235/352/674/699, 170/235/352/699, 170/235/412, 170/235/645, 170/235/660, 170/252/352/423/674/699, 170/269/352/423/476/605/659/660/699, 194, 194/198/252/319/423/645/674, 194/235/252, 194/252/269/319/434/605/645/659/674/699, 194/269/319/412/605/674/699, 194/319/352/605/660, 194/352/605/645/699, 194/645/659, 197, 217, 227, 235/249/252/323/352/476, 235/249/252/423/699, 235/252, 235/252/269/423/605/645/660/674, 235/269/323/412/434/659/674/699, 235/352, 235/352/605, 235/605/645, 249/252/319/605/660, 252/645, 255, 255/391/472/557/660/743, 255/391/476/513/557/602/685/758, 255/391/476/557/658, 255/391/557, 255/398/472/476/557/658, 255/398/476/557/658/660/685, 255/476, 255/660, 268, 269/352, 269/352/412, 269/605, 319, 319/352/412/434/699, 319/434/476, 329, 331, 339, 352, 352/412/605/645/699, 354, 358, 360, 391, 391/398/476/685, 391/451/472/513/557, 391/472/476/557/602, 391/472/476/557/658/660/743/758, 391/557, 391/581, 412, 412/645, 412/674, 423/476/645/674, 423/645, 467, 478/557, 506, 540, 557, 557/658/660, 557/658/685, 602/701, 602/703, 605/699, 658, 658/660, 660, 699, 713, and 743, wherein the amino acid positions are relative to the reference sequence of SEQ ID NO: 2.

5. An engineered DNA polymerase or a functional fragment thereof, comprising a polypeptide sequence having at least 75% or more sequence identity to a reference sequence comprising residues 12 to 841 of SEQ ID NO: 178, or to a reference sequence of SEQ ID NO: 178, wherein the polypeptide sequence comprises one or more substitutions relative to the reference sequence comprising residues 12 to 841 of SEQ ID NO: 178, or to the reference sequence of SEQ ID NO: 178.

6. The engineered DNA polymerase of claim 5, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 178 or to the reference sequence of SEQ ID NO: 178.

7. The engineered DNA polymerase of claim 5, comprising one or more substitutions or substitution sets selected from 12, 14, 15, 17, 19, 36, 39, 44, 45, 82, 86, 87, 95, 122, 139, 139/395, 159, 167, 170/194, 170/194/423/451/557/658/660/743, 170/194/423/476/743, 170/194/423/557/658/659/743, 170/194/423/557/658/660/685, 170/194/423/557/658/743, 170/194/451/476/557/658, 170/194/451/476/557/658/659/660/743, 170/194/451/476/557/658/685, 170/194/451/557/659/660, 170/194/476/557/659, 170/194/557, 170/194/658, 170/194/658/659/660/685/743, 170/194/658/743, 170/423, 170/423/451/476/557/658/659/743, 170/423/451/557, 170/423/451/557/658/659/685/743, 170/423/451/557/659, 170/423/476, 170/423/476/658/659, 170/423/476/658/659/660/685/743, 170/423/476/658/660/685/743, 170/423/476/658/685, 170/423/476/658/685/743, 170/423/476/685, 170/423/476/685/743, 170/423/557/658/659, 170/423/557/658/660/743, 170/423/557/658/743, 170/423/557/659/660/685/743, 170/423/557/660/743, 170/423/557/685, 170/423/557/685/743, 170/423/658, 170/423/658/659/743, 170/423/658/660, 170/423/743, 170/451/557, 170/451/557/658/660, 170/476/557/658/659, 170/476/557/658/685, 170/476/557/743, 170/476/659, 170/557, 170/557/658/660/685/687, 170/658, 170/658/659, 170/658/659/743, 170/659, 170/660, 179, 194/423/451/557/658/659/743, 194/423/476/658/660/685, 194/423/557/658/659/743, 194/423/685, 194/451/476/557/658/659, 194/451/476/557/658/659/660/743, 194/557, 194/557/658/659/685, 194/557/659/743, 194/658/659/685, 194/658/660, 197, 198, 198/423/476/557/743, 205, 209, 214, 222, 228, 249, 250/269/331, 252, 252/258/331, 252/258/331/352/467/506/674, 252/269, 252/269/331/339/412/413, 252/269/331/352/412/674/704, 252/269/331/506/674, 252/269/339/352/356/660/704, 252/269/339/352/412/699/704, 252/269/352/660, 252/269/412/674, 252/269/413/467/660/699/704, 252/269/467/506, 252/269/674, 252/331, 252/331/339/352/467/506, 252/331/339/412/413/467/699, 252/331/352/412/413/467/660/704, 252/331/352/412/506/674, 252/331/358/506, 252/331/412/413, 252/331/412/413/674, 252/331/412/467/506/699/704, 252/331/412/506/674, 252/331/412/506/704, 252/331/413, 252/331/467/660/674, 252/331/660, 252/331/699, 252/339/352/467/674/699, 252/339/352/674/704, 252/339/412/413/467/674, 252/339/412/413/674/699/704, 252/339/412/467/674, 252/339/412/674/704, 252/339/467/506/699/704, 252/341/412/413, 252/341/412/674, 252/352/412, 252/352/506, 252/352/660, 252/358/674, 252/412, 252/412/413, 252/412/467, 252/412/467/660/699/704, 252/413, 252/413/506/660/704, 252/413/699, 252/467, 252/467/506/674/704, 252/506/704, 252/674, 252/699, 252/704, 264, 268, 268/510, 268/621, 269, 269/331/352/412/467/506/704, 269/331/352/467/506/699, 269/331/352/467/699, 269/331/412/413/467/506/660/674, 269/331/413/506, 269/331/467/506/660, 269/331/660/699, 269/331/674/699, 269/331/699, 269/331/699/704, 269/339/506/704, 269/352/467/506/674, 269/358, 269/358/412/660/704, 269/358/467/506/660/699, 269/412/413/506/704, 269/412/413/674, 269/412/467/704, 269/467/506, 269/506, 269/599/660/674/699, 269/674, 269/699/704, 269/704, 272/352/506, 275, 301, 323, 331, 331/339/412/413/467/506/674/704, 331/339/412/467/506/704, 331/352/358/699/704, 331/352/412/413/506, 331/358, 331/358/467/674/699/704, 331/412, 331/412/413/467/699/704, 331/412/467/674/704, 331/412/467/704, 331/412/660/699, 331/413/467/660/674, 331/467, 331/467/506, 331/467/674/699, 331/506, 331/506/674/699, 331/660/674/699, 331/674, 331/674/699, 331/699, 331/704, 339, 339/352, 339/352/412/419/467/506/660/674/699, 339/352/412/467/660, 339/352/412/660/674, 339/352/674/699, 339/412/413, 339/412/413/467/674/699, 339/412/467/674, 339/467/674, 339/506, 339/506/699, 339/506/704, 339/660, 339/660/674, 341/352/704, 341/467, 341/467/506/660/674, 341/467/674, 345, 352, 352/358, 352/412/413/467/506/699, 352/413/467/506, 352/413/674, 352/660, 352/674, 355, 358/412, 358/413, 358/674, 412, 412/413/467/660, 412/413/467/674, 412/467/506/674/704, 412/467/660/674/699, 412/660, 412/674, 412/674/699, 413/467, 413/506, 413/506/704, 413/674/699/704/740, 423, 423/451/476/557/658/685, 423/451/476/658/685, 423/451/557, 423/451/557/658, 423/451/557/658/685, 423/476, 423/476/557/658/659/660, 423/476/557/658/659/685/743, 423/476/557/659/660/685/743, 423/476/557/659/660/743, 423/476/658/660/743, 423/476/658/685/743, 423/476/658/743, 423/557/658, 423/557/658/659/660, 423/557/658/659/660/685, 423/557/658/660/743, 423/557/685, 423/557/685/743, 423/658, 423/658/659/660, 423/658/660, 423/660, 423/685, 423/685/743, 423/743, 428, 433, 433/497, 467, 467/506/699/704, 467/658/660/674/699, 470, 476, 476/480/557/659, 476/557/658/659, 476/557/658/660/685, 476/557/659, 476/557/659/660, 476/658/659/660/685/743, 476/658/660/743, 477, 478, 485, 489/557/743, 492, 501, 506/660, 506/674/699/704, 506/674/704, 511, 514, 517, 520, 525/557/659/660/743, 533, 534, 534/670, 549, 557, 557/658, 557/658/659, 557/658/659/660, 557/659/660, 557/660, 557/743, 565, 596, 598, 642, 658, 658/659, 658/659/660/685/743, 658/659/685, 658/743, 659/660, 659/660/685/743, 674, 674/699, 685, 689, 695, 696, 699, 699/704, 704, 707, 725, 743, 745, 747, 783, and 821, or combinations thereof, wherein the amino acid positions are relative to the reference sequence of SEQ ID NO: 178.

8. The engineered DNA polymerase of claim 5, comprising one or more substitutions or substitution sets selected from 12, 13, 14, 15, 17, 19/214, 45, 84, 87, 95, 122, 159, 170/194/476/557/659, 170/423/476/658/685, 170/423/557/658/743, 170/476/557/658/659, 170/476/557/743, 178, 194/658/660, 198, 209, 214, 222, 228, 249, 252, 252/258/331, 252/269, 252/269/412/674, 252/269/674, 252/331, 252/331/352/412/506/674, 252/331/467/660/674, 252/331/660, 252/331/699, 252/352/412, 252/412, 252/412/467/660/699/704, 252/413, 252/467/506/674/704, 252/704, 264, 268, 268/621, 269/331/699, 269/412/467/704, 269/467/506, 269/674, 269/695, 269/699/704, 275, 276, 301, 323, 331/358, 331/412/660/699, 331/674, 331/704, 345, 352/674, 412/467/660/674/699, 412/660, 412/674, 430, 430/837, 433, 467, 470, 476/557/658/659, 476/658/660/743, 477, 478, 485, 492, 511, 514, 517, 520, 533, 534, 534/670, 549, 557/658/659, 565, 596, 598, 642, 658/659, 674, 689, 695, 696, 699/704, 707, 725, and 783, or combinations thereof, wherein the amino acid positions are relative to the reference sequence of SEQ ID NO: 178.

9. The engineered DNA polymerase of claim 5, comprising one or more substitutions or substitution sets selected from 12, 13, 14, 15, 17, 84, 87, 122, 159, 170/423/476/658/685, 170/476/557/743, 178, 198, 205, 209, 222, 228, 249, 252, 252/269, 252/269/412/674, 252/269/674, 252/331, 252/331/352/412/506/674, 252/331/467/660/674, 252/331/699, 252/412, 252/412/467/660/699/704, 252/413, 252/467/506/674/704, 252/704, 264, 269/331/699, 269/412/467/704, 269/467/506, 269/674, 275, 276, 301, 322, 323, 331/358, 331/412/660/699, 331/674, 331/704, 345, 352/674, 355, 412/674, 430, 430/837, 433, 467, 470, 476/658/660/743, 477, 478, 485, 492, 511, 514, 517, 520, 533, 534, 534/670, 549, 557/658/659, 565, 596, 598, 642, 674, 689, 695, 696, 699/704, 703, 725, and 821, or combinations thereof, wherein the amino acid positions are relative to the reference sequence of SEQ ID NO: 178.

10. The engineered DNA polymerase of claim 5, comprising one or more substitutions or substitution sets selected from 12, 15, 17, 252, 252/331, 252/331/352/412/506/674, 252/413, 252/704, 269/412/467/704, 269/467/506, 269/674, 301, 331/704, 345, 352/674, 355, 430/837, 492, 511, 514, 520, 598, 642, 674, 695, 696, 699/704, and 703, or combinations thereof, wherein the amino acid positions are relative to the reference sequence of SEQ ID NO: 178.

11. An engineered DNA polymerase or a functional fragment thereof, comprising a polypeptide sequence having at least 75% or more sequence identity to a reference sequence comprising residues 12 to 841 of SEQ ID NO: 994, or to a reference sequence of SEQ ID NO: 994, wherein the polypeptide sequence comprises one or more substitutions relative to the reference sequence comprising residues 12 to 841 of SEQ ID NO: 994, or to the reference sequence of SEQ ID NO: 994.

12. The engineered DNA polymerase of claim 11, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 994 or to the reference sequence of SEQ ID NO: 994.

13. The engineered DNA polymerase of claim 11, comprising a substitution or substitution set selected from 2, 5, 7, 8, 12/15/269/355/412/413/492/704, 12/252/413/492, 32, 33, 35/304, 38, 40, 65, 80, 81, 140, 151, 169, 194, 194/252, 194/252/358, 194/252/358/412/467, 194/252/358/412/467/658/696/743, 194/252/412/696, 194/252/467/696/743, 194/252/743, 194/358, 194/358/412, 194/358/658, 194/358/704, 194/412, 194/412/467, 194/412/467/743, 194/467/511/658, 194/467/658, 194/467/704, 194/467/743, 194/743, 218, 226, 242, 252, 252/412/658, 252/467/696, 258/347, 279, 284, 289, 298, 300, 304, 311, 329, 331, 340, 358, 375, 375/376, 406, 420, 434, 440, 481, 489, 490, 518, 529, 543, 560, 592, 635, 639, 645, 658, 658/704, 666, 685, 694, 704, 713, 717, 718, 721, 740, 746, 782, 804, 807, 814, and 829, or combinations thereof, wherein the amino acid positions are relative to the reference sequence of SEQ ID NO: 994.

14. The engineered DNA polymerase of claim 11, comprising one or more substitutions or substitution sets selected from 2, 5, 8, 12/15/269/355/412/413/492/704, 12/252/413/492, 38, 40, 65, 80, 140, 151, 218, 226, 230, 242, 258/347, 279, 284, 289, 298, 300, 304, 309, 311, 329, 331, 340, 375, 375/376, 406, 420, 434, 441, 489, 490, 518, 529, 543, 560, 592, 635, 639, 645, 658, 666, 685, 704, 713, 717, 718, 721, 740, 743, 746, 782, 804, 807, 814, and 829, or combinations thereof, wherein the amino acid positions are relative to the reference sequence of SEQ ID NO: 994.

15. The engineered DNA polymerase of claim 11, comprising one or more substitutions or substitution sets selected from 2, 5, 8, 12/15/269/355/412/413/492/704, 12/252/413/492, 32, 38, 40, 65, 98, 140, 151, 169, 202, 218, 226, 227, 230, 242, 258/347, 279, 284, 298, 300, 304, 309, 311, 329, 331, 340, 375, 375/376, 406, 420, 434, 440, 441, 481, 489, 490, 518, 529, 543, 560, 592, 635, 639, 645, 658, 666, 685, 692, 694, 704, 713, 717, 718, 721, 740, 746, 782, 804, 807, 814, and 829, or combinations thereof, wherein the amino acid positions are relative to the reference sequence of SEQ ID NO: 994.

16. The engineered DNA polymerase of claim 11, comprising one or more substitutions or substitution sets selected from 12/15/269/355/412/413/492/704, 12/252/413/492, 194, 194/252, 194/252/358/412/467, 194/252/358/412/467/658/696/743, 194/252/412/696, 194/252/467/658, 194/252/467/696/743, 194/252/467/704, 194/252/696/704, 194/252/704/743, 194/252/743, 194/358, 194/358/412, 194/358/467/658/696/704, 194/358/658, 194/412, 194/412/467, 194/412/658, 194/467, 194/467/658, 194/467/658/743, 194/467/704, 194/467/743, 194/658, 194/743, 242, 252/358/696/704, 252/412/658, 252/467/696, 258/347, 284, 304, 331, 434, 489, 543, 560, 592, 658, 666, 685, 717, 721, and 814, or combinations thereof, wherein the amino acid positions are relative to the reference sequence of SEQ ID NO: 994.

17. An engineered DNA polymerase or a functional fragment thereof, comprising a polypeptide sequence having at least 75% or more sequence identity to a reference sequence comprising residues 12 to 841 of SEQ ID NO: 1858, or to a reference sequence of SEQ ID NO: 1858, wherein the polypeptide sequence comprises one or more substitutions relative to the reference sequence comprising residues 12 to 841 of SEQ ID NO: 1858, or to the reference sequence of SEQ ID NO: 1858.

18. The engineered DNA polymerase of claim 17, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 1858 or to the reference sequence of SEQ ID NO: 1858.

19. The engineered DNA polymerase of claim 17, comprising one or more substitutions or substitution sets selected from 194/252/269/304/467, 194/252/304, 194/252/304/358/467/658, 194/252/304/467, 194/252/358/489, 194/252/467, 194/269, 194/269/304, 194/269/304/658/717, 194/269/717, 194/304/358/467/489, 194/467/721, 194/536/658, 194/721, 252, 252/269/304/743, 252/304/489/696/717, 252/304/658, 252/358/467/489/685, 252/685, 269, 269/304, 269/304/467, 269/304/658, 269/658, 269/721, 304, 304/358/467/592, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

20. The engineered DNA polymerase of claim 17, comprising one or more substitutions or substitution sets selected from 252, 252/304, 252/304/489/696/717, 252/304/658, 252/358/467/489/685, 252/685, 269, 269/304/467, 269/304/489/685, 269/467/489/658/685, 269/658, 269/721, 304, 304/467/489/717, 304/489/658, 304/489/658/721, 304/685, 358/658, 489, 489/696/743, 685, 696, 717, and 721, or combinations thereof, wherein the amino acid positions are relative to the reference sequence of SEQ ID NO: 1858.

21. The engineered DNA polymerase of claim 17, comprising one or more substitutions or substitution sets selected from 252/685, 269, 269/658, 304/467/489/717, 304/489/658/721, 304/685, 358/658, 489, 685, 696, 717, and 721, or combinations thereof, wherein the amino acid positions are relative to the reference sequence of SEQ ID NO: 1858.

22. An engineered DNA polymerase or a functional fragment thereof, comprising a polypeptide sequence having at least 75% or more sequence identity to a reference sequence comprising residues 12 to 841 of SEQ ID NO: 2176, or to a reference sequence of SEQ ID NO: 2176, wherein the polypeptide sequence comprises one or more substitutions relative to the reference sequence comprising residues 12 to 841 of SEQ ID NO: 2176, or to the reference sequence of SEQ ID NO: 2176.

23. The engineered DNA polymerase of claim 22, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2176 or to the reference sequence of SEQ ID NO: 2176.

24. The engineered DNA polymerase of claim 22, comprising one or more substitutions or substitution sets selected from 40, 42, 45, 45/65/243/268, 45/109/340/605/814, 45/221/243/340/423, 45/243/312/331/451, 45/243/312/331/557, 45/243/312/340/423/543/814, 45/243/814, 45/268/312/605/748/749/814, 45/268/489/557/748/749, 45/312/340/605, 45/340/423/814, 45/340/557, 45/423, 45/431, 45/451, 45/475/557/748/749, 45/489/557/749, 45/621, 45/699/748/749, 57, 61, 64/293, 65, 65/233/543/621, 65/489/543, 81, 94, 102, 109, 109/340/489/510/659, 118, 124, 145, 151, 177, 186, 194, 194/269/489, 194/358, 210, 211, 221, 223, 227, 227/443/592/617, 227/443/756, 233, 242, 243, 243/268, 252, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 289, 292, 292/430, 298, 300, 301, 301/538/546/621/814, 302, 302/340/489/494/510/748/814, 302/340/494, 302/494, 302/494/525, 302/494/543/659/814, 304/489, 312, 312/331/605/814, 312/340/494, 316, 323, 329, 331, 331/605, 340, 340/489, 340/489/494/543/659/814, 340/489/699, 350, 352, 352/451/489/543/557/721, 355, 355/430, 358/467, 371, 397, 406, 407, 420, 423, 431, 441, 451, 463, 466, 475, 481, 488, 489, 489/494, 489/510, 489/621/814, 489/658, 492, 494, 501, 510, 510/748, 519, 521, 530, 533, 534, 536, 538, 543, 543/699/814, 553, 557, 557/605/699/721, 560, 597, 605, 621, 639, 644, 659, 660, 666, 670, 685, 686, 692, 694, 696, 699, 704, 717, 721, 722, 726, 732, 738, 743, 748, 748/749, 749, 756, 802, 804, 814, 827, 829, and 834, or combinations thereof, wherein the amino acid positions are relative to the reference sequence of SEQ ID NO: 2176.

25. The engineered DNA polymerase of claim 22, comprising one or more substitutions or substitution sets selected from 45, 45/65/243/268, 45/109/243/423/543/605/699/748/749/814, 45/109/340/605/814, 45/221/243/340/423, 45/243/312/331/451, 45/243/312/331/557, 45/243/312/340/423/543/814, 45/243/312/605, 45/243/814, 45/268/312/605/748/749/814, 45/268/489/543/557/659/721, 45/268/489/557/748/749, 45/312, 45/312/340/605, 45/340/423/814, 45/340/475/557/814, 45/340/557, 45/423, 45/431, 45/451, 45/475/557/748/749, 45/489/557/749, 45/621, 45/699/748/749, 64/293, 65, 65/489/543, 81, 94, 102, 109, 109/340/489/510/659, 109/423, 118, 120, 151, 162, 194, 211, 221, 233, 243, 243/268, 245, 252, 258, 268, 268/312/331/340/699, 268/312/331/423/451/557/605, 268/340/352/423/451, 268/423, 268/423/431/543/557/605/814, 268/423/814, 268/431/605/749, 269, 269/658, 272, 280, 283, 284, 292, 298, 300, 301, 301/538/546/621/814, 302/340/489/494/510/748/814, 302/340/489/494/525, 302/340/494, 302/494, 302/494/525, 304/489, 312, 312/331/605/814, 312/423/431/451/489, 312/494/525/543/659/748/814, 323, 329, 331, 331/605, 340, 340/489, 340/489/494/543/659/814, 340/489/699, 350, 352, 352/451/489/543/557/721, 355, 355/430, 358/467, 406, 407, 423, 423/463/605, 431, 451, 463, 466, 467, 475, 476, 481, 489, 489/494, 489/494/659, 489/510, 489/658, 494, 501, 510, 510/748, 519, 530, 533, 534, 538, 543, 543/699/814, 557, 557/605/699/721, 560, 597, 605, 621, 659, 666, 694, 696, 699, 721, 722, 748, 748/749, 749, 756, 802, and 814, or combinations thereof, wherein the amino acid positions are relative to the reference sequence of SEQ ID NO: 2176.

26. The engineered DNA polymerase of claim 22, comprising one or more substitutions or substitution sets selected from 45, 45/65/243/268, 45/221/243/340/423, 45/268/312/605/748/749/814, 45/268/489/557/748/749, 45/451, 45/475/557/748/749, 45/489/557/749, 45/621, 45/699/748/749, 65, 65/489/543, 81, 94, 102, 109, 109/340/489/510/659, 221, 227/443/756, 252, 268, 268/312/331/340/699, 268/312/331/423/451/557/605, 268/423/431/543/557/605/814, 269, 283, 300, 301, 301/538/546/621/814, 302/340/489/494/510/748/814, 302/340/489/494/525, 302/494/525, 304/489, 312, 312/494/525/543/659/748/814, 329, 331, 340, 340/489/699, 352, 358/467, 406, 431, 451, 463, 466, 475, 481, 489, 489/510, 489/658, 510, 510/748, 530, 543, 543/699/814, 557, 557/605/699/721, 560, 605, 659, 666, 696, 699, 721, 722, 748, 748/749, 749, 802, and 814, or combinations thereof, wherein the amino acid positions are relative to the reference sequence of SEQ ID NO: 2176.

27. The engineered DNA polymerase of claim 22, comprising one or more substitutions or substitution sets selected from 45/65/243/268, 45/268/312/605/748/749/814, 45/268/489/557/748/749, 45/451, 45/475/557/748/749, 45/489/557/749, 45/621, 45/699/748/749, 57, 65, 65/489/543, 81, 94, 109, 221, 243, 268, 268/312/331/340/699, 268/312/331/423/451/557/605, 268/423/431/543/557/605/814, 269, 300, 301, 302/340/489/494/510/748/814, 302/494/525, 304/489, 312, 331, 331/605, 340, 340/489/699, 352, 358/467, 406, 423, 431, 451, 463, 466, 475, 481, 489, 489/510, 489/658, 494, 510, 510/748, 530, 543, 543/699/814, 557, 557/605/699/721, 560, 597, 605, 659, 666, 696, 699, 721, 748, 748/749, 749, 802, and 814, or combinations thereof, wherein the amino acid positions are relative to the reference sequence of SEQ ID NO: 2176.

28. An engineered DNA polymerase or a functional fragment thereof, comprising a polypeptide sequence having at least 75% or more sequence identity to a reference sequence comprising residues 12 to 841 of SEQ ID NO: 2310, or to a reference sequence of SEQ ID NO: 2310, wherein the polypeptide sequence comprises one or more substitutions relative to the reference sequence comprising residues 12 to 841 of SEQ ID NO: 2310, or to the reference sequence of SEQ ID NO: 2310.

29. The engineered DNA polymerase of claim 28, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2310 or to the reference sequence of SEQ ID NO: 2310.

30. The engineered DNA polymerase of claim 28, comprising one or more substitutions or substitution sets selected from 18/268/302/312/605/659/699, 45/227/268/423/489/510/605, 45/227/302/423/510/605/621/699, 45/227/312/423/525/605/699, 45/268/302/423/494/621/699, 45/268/443/525/696, 45/331/489, 45/331/621, 45/340/423, 45/494/525/621/696/699, 45/605, 45/696/699, 65/685, 227, 227/268/340/451, 227/268/423/621, 227/302, 227/312/489/494/621, 227/331, 227/331/423/605, 227/331/605/659, 227/340/423/605, 227/340/621, 227/423, 227/423/451, 227/451/525/621/659/699/721, 227/525, 227/543/659/699, 227/605/721, 227/699, 268/302/340/358/605/699, 268/302/423/605, 268/340, 268/340/423/443/525/621/659, 268/340/423/621, 268/340/525/605, 268/423, 268/423/478/489/525/605/699, 268/423/489/494/605/659, 268/423/510/605, 268/423/605/699, 268/423/699, 268/467, 268/467/489, 268/467/489/510/699, 268/467/489/699, 268/467/489/699/814, 268/467/510/699/814, 268/489/494/605/699, 268/489/699, 268/510, 268/510/699/814, 268/525, 268/525/605, 268/543, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/451, 331/525/605/699, 331/605/699, 340/451/494/525, 358/467/489/699, 358/467/699, 358/467/699/814, 423/443/451/494/510, 423/451/605/659/699, 423/489/494/529/605/621/696/699, 423/494, 423/525, 423/605/621, 451/510, 467, 467/489, 467/489/510/543/699/814, 467/489/699, 467/489/699/814, 467/489/814, 467/510/699, 467/510/699/814, 467/510/814, 467/699, 484, 489, 489/494/605/721, 489/510/525/605/699, 489/699, 489/699/814, 494/605/699, 494/659, 494/699, 498, 503, 510, 510/605/659, 510/699, 510/699/814, 524, 526, 527, 531, 540, 542, 543/699, 543/699/814, 547, 569, 577/614, 605, 605/621/659/696/699, 605/621/696/699, 621, 659/699, 673, 684, 685, 685/714, 699, 699/814, 702, and 716, or combinations thereof, wherein the amino acid positions are relative to the reference sequence of SEQ ID NO: 2310.

31. The engineered DNA polymerase of claim 28, comprising one or more substitutions or substitution sets selected from 18/268/302/312/605/659/699, 45/227/268/423/489/510/605, 45/227/302/423/510/605/621/699, 45/227/312/423/525/605/699, 45/268/302/423/494/621/699, 45/494/525/621/696/699, 227, 227/268/340/451, 227/268/423/621, 227/302, 227/312/489/494/621, 227/331, 227/331/423/605, 227/331/605/659, 227/340/423/605, 227/340/621, 227/423, 227/543/659/699, 227/605/721, 268, 268/302, 268/302/423/605, 268/340/423/443/525/621/659, 268/423/510/605, 268/423/605/699, 268/467, 268/467/489, 268/467/489/510/699, 268/467/489/699, 268/489/699, 268/510, 268/510/699/814, 268/525/605, 268/543/699, 268/605/621/721, 268/621, 268/699, 302/423, 302/423/605/621, 302/543/621/696/699, 302/605/621/721, 312/331/451/621/696/699, 312/423/621, 312/510/605, 331/423, 331/423/527, 331/605, 331/605/699, 340/451/494/525, 358/467/489/699, 358/467/699/814, 423, 423/443/451/494/510, 423/489/494/529/605/621/696/699, 423/605/621, 451/510, 467/489, 467/489/510/543/699/814, 467/489/699, 467/489/814, 467/510/699, 467/510/699/814, 467/699, 467/699/814, 484, 489, 489/510/525/605/699, 489/699, 489/699/814, 494/605/699, 498, 503, 510, 510/605/659, 510/699, 510/699/814, 524, 526, 543/699, 543/699/814, 605/621/659/696/699, 605/621/696/699, 621, and 699/814, or combinations thereof, wherein the amino acid positions are relative to the reference sequence of SEQ ID NO: 2310.

32. The engineered DNA polymerase of claim 28, comprising one or more substitutions or substitution sets selected from 18/268/302/312/605/659/699, 45/227/268/423/489/510/605, 45/227/302/423/510/605/621/699, 45/494/525/621/696/699, 227/331/605/659, 227/605/721, 268, 268/302/423/605, 268/423/605/699, 268/467, 268/467/489, 268/467/489/510/699, 268/467/489/699, 268/489/699, 268/510, 268/605/621/721, 302/543/621/696/699, 312/331/451/621/696/699, 312/510/605, 331/423/527, 340/451/494/525, 423/489/494/529/605/621/696/699, 423/605/621, 467/489/510/543/699/814, 467/489/699, 467/489/814, 467/510/699/814, 489/510/525/605/699, 489/699, 489/699/814, 494/605/699, 510, 510/699/814, 543/699, and 543/699/814, or combinations thereof, wherein the amino acid positions are relative to the reference sequence of SEQ ID NO: 2310.

33. The engineered DNA polymerase of claim 28, comprising one or more substitutions or substitution sets selected from 18/268/302/312/605/659/699, 45/227/268/423/489/510/605, 45/227/302/423/510/605/621/699, 65/685, 227/331/605/659, 227/605/721, 268, 268/302/423/605, 268/423/605/699, 268/467, 268/467/489, 268/467/489/699, 268/489/699, 268/510, 268/605/621/721, 302/543/621/696/699, 312/331/451/621/696/699, 312/510/605, 423/605/621, 467/489/510/543/699/814, 467/489/699, 467/489/814, 467/510/699/814, 489/699, 489/699/814, 510, 510/699/814, 542, 543/699, 543/699/814, 685, and 685/714, or combinations thereof, wherein the amino acid positions are relative to the reference sequence of SEQ ID NO: 2310.

34. An engineered DNA polymerase or a functional fragment thereof, comprising a polypeptide sequence having at least 75% or more sequence identity to a reference sequence comprising residues 12 to 841 of SEQ ID NO: 2994, or to a reference sequence of SEQ ID NO: 2994, wherein the polypeptide sequence comprises one or more substitutions relative to the reference sequence comprising residues 12 to 841 of SEQ ID NO: 2994, or to the reference sequence of SEQ ID NO: 2994.

35. The engineered DNA polymerase of claim 34, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 2994 or to the reference sequence of SEQ ID NO: 2994.

36. The engineered DNA polymerase of claim 34, comprising one or more substitutions or substitution sets selected from 4, 6, 10, 11, 12, 14, 14/50, 14/50/56/598/725, 14/50/598/695, 14/95, 14/95/501/642, 14/139/249/433, 14/139/725, 14/167, 14/167/197/433, 14/167/197/501/725, 14/167/197/642/695, 14/167/249/433/598/642, 14/167/249/501/598/642, 14/167/249/598/695/725, 14/167/249/642, 14/167/642, 14/167/695, 14/167/725, 14/197/249/642, 14/197/642, 14/249/433/725, 14/249/598, 14/249/642, 14/249/725, 14/433/598, 14/501, 14/598, 14/598/695, 14/598/695/725, 14/642/725, 18, 18/302, 18/302/531/673/684, 18/302/547, 18/484/673, 18/484/673/684, 18/524/547, 18/547, 18/684, 29, 45/268/299/302/321/621/659, 45/268/302/621/721, 45/376/385/408/494/525/621/696, 45/423, 48, 50, 50/433/598, 50/501, 50/598, 50/598/695, 50/642, 53, 76, 77, 91, 95/167/197/598/642/695, 95/167/197/598/695/725, 95/249/433/642, 95/249/598, 95/598, 99, 105, 122, 139/167, 139/167/197/433/501/642, 139/433/598, 141, 167, 167/197, 167/197/642/695/725, 167/642, 167/725, 177, 197/642/725, 197/695, 202, 204, 209, 217, 227, 239, 247, 249/695, 249/725, 266, 268, 268/302/451/494/621, 270, 291, 291/684, 299, 299/474, 302, 302/423/451/494/621, 302/423/621, 302/451/494, 313, 346, 349, 356, 361, 370, 374, 377, 394, 404, 423, 423/451/621/696, 423/494/525, 426, 426/798, 427, 433/501/725, 436, 439/509/789, 440, 444, 445, 445/446, 446/811, 448, 448/793, 459, 461, 482, 484, 484/684, 485, 489, 494, 498/684, 509, 514, 517, 525/529/621, 533, 543/621/721, 547, 565, 574, 596, 598, 598/642, 598/642/695, 598/695, 598/725, 601, 610, 621, 621/659, 636, 642, 642/692, 659, 662, 673, 683, 684, 688, 689, 690, 695, 725, 741, 744, 744/793, 751, 801, 810, 818, 830, and 832, or combinations thereof, wherein the amino acid positions are relative to the reference sequence of SEQ ID NO: 2994.

37. The engineered DNA polymerase of claim 34, comprising one or more substitutions or substitution sets selected from 4, 6, 10, 11, 14, 14/50, 14/139/725, 14/167, 14/167/642, 14/167/695, 14/598, 18/302/531/673/684, 18/524/547, 18/684, 45/268/299/302/321/621/659, 45/268/302/621/721, 48, 50/598, 77, 95/167/197/598/695/725, 99, 105, 122, 167, 167/197, 177, 209, 217, 239, 249/695, 268, 346, 349, 356, 423, 423/451/621/696, 426, 426/798, 427, 433/501/725, 436, 439/509/789, 445, 448, 448/793, 482, 484, 484/684, 514, 517, 533, 543/621/721, 565, 574, 598/695, 621, 621/659, 659, 662, 684, 688, 690, 725, 744/793, 801, 810, and 832, or combinations thereof, wherein the amino acid positions are relative to the reference sequence of SEQ ID NO: 2994.

38. The engineered DNA polymerase of claim 34, comprising one or more substitutions or substitution sets selected from 11, 12, 18/547, 45/268/299/302/321/621/659, 45/268/302/621/721, 45/376/385/408/494/525/621/696, 48, 105, 122, 302, 346, 426/798, 439/509/789, 448/793, 484, 514, 525/529/621, 533, 543/621/721, 574, 690, and 744/793, or combinations thereof, wherein the amino acid positions are relative to the reference sequence of SEQ ID NO: 2994.

39. The engineered DNA polymerase of claim 34, comprising one or more substitutions or substitution sets selected from 11, 12, 18, 18/302, 18/302/547, 18/547, 45/268/299/302/321/621/659, 45/268/302/621/721, 45/376/385/408/494/525/621/696, 48, 105, 122, 291/684, 302, 346, 426/798, 439/509/789, 448/793, 484, 514, 525/529/621, 533, 547, 574, 684, 690, and 744/793, or combinations thereof, wherein the amino acid positions are relative to the reference sequence of SEQ ID NO: 2994.

40. An engineered DNA polymerase or a functional fragment thereof, comprising a polypeptide sequence having at least 75% or more sequence identity to a reference sequence comprising residues 12 to 841 of SEQ ID NO: 3796, or to a reference sequence of SEQ ID NO: 3796, wherein the polypeptide sequence comprises one or more substitutions relative to the reference sequence comprising residues 12 to 841 of SEQ ID NO: 3796, or to the reference sequence of SEQ ID NO: 3796.

41. The engineered DNA polymerase of claim 40, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 3796 or to the reference sequence of SEQ ID NO: 3796.

42. The engineered DNA polymerase of claim 40, comprising one or more substitutions or substitution sets selected from 1/2/149, 15/167, 18, 18/105/139/197/249/426/598, 18/105/139/197/291/299/423/426/448/529/547/725, 18/105/249/284/302/423/426/501/673/684/798, 18/105/321/547, 18/139/197/249/299/302/433/547/642/659/695/744, 18/139/197/426/598, 18/139/197/642, 18/139/249/423/433/501/547/725, 18/197/249/291/484, 18/197/321/426/448/451/659/673/744, 18/197/321/433/484, 18/249, 18/249/299/302/423/529/533/642/659/673, 18/249/302/423/484/547/695, 18/249/448/484/673, 18/249/673, 18/291/302/423/684, 18/291/302/433, 18/291/302/525/547/659/684/695, 18/291/302/684, 18/291/423/525/529, 18/291/659, 18/291/695, 18/299/302/423/426/525/529, 18/299/451/525/529/695, 18/302/423/529, 18/302/433/547/684, 18/302/525/529/547, 18/302/529/659/695, 18/321/423, 18/321/484/547/725, 18/423, 18/423/529/695, 18/433, 18/433/673/725, 18/484, 18/598/725, 32, 38/463, 64, 105/139/291/302/423/484/547/725, 105/139/448/501/525/529/642/659, 105/197/249/423/426/547/642/673, 105/321/433/744, 105/321/501, 137, 139/249/291/321/484/695/725, 139/249/291/451/744, 139/249/426/451/501/673/744, 149, 160, 164/172, 167, 172, 197/249/327/423/673/690, 197/302/391/547/598, 197/525/529/659/744, 197/547, 197/684/725, 200/577, 245/423, 247/423/448, 249/321/673/798, 249/423/426/673, 249/448/451/547/659/725, 249/725/744/798, 253, 256, 256/455, 271, 281, 291/302/423, 291/302/529, 291/321/423/725, 291/423/525, 291/423/529/533/598/642/725/798, 291/484/598/642/673/744/798, 291/525, 300, 302, 302/364/376/383/386/408/451/484/598, 302/423/433/529/695, 302/423/525/529, 302/423/547/684/695, 302/684/695, 311, 314, 316, 321/448/529/673, 323, 330, 334/431, 347, 348, 352/363, 353, 354, 358, 361, 363, 368, 372, 376, 376/699, 383, 389, 392, 394, 411, 414, 422, 423/424/547, 423/433/484/529/533/642, 423/433/487/525/547, 423/484/673, 423/529/684, 423/547/598/642, 430, 431, 433/525/529/659/684/695, 436/743, 448/451/529/725, 451/598/725, 455, 455/518, 462, 463, 464, 468, 469, 472, 474, 474/821, 476, 479, 480, 484, 489, 491, 492, 498, 501, 504/548, 505, 510, 519/720, 521, 524, 525, 525/533, 529, 531, 532, 533, 535, 540, 546, 547, 550, 553, 555, 555/572, 557, 558, 559, 566, 568, 570, 571, 572, 573, 574, 575, 576, 577, 583, 588, 590, 590/652, 592, 597, 600, 601, 612, 615, 618, 620, 622, 623, 625, 628, 643, 654, 655, 660, 664, 675, 677, 682, 685, 690, 691/692, 692, 692/766, 697, 703, 705, 708, 711, 715, 720, 726, 728, 730, 732, 738, 743, 746, 752, 753, 756, 766, 769, 770, 779, 793, 800, 802, 803, 805, 813, 819, 825, 827, and 829, or combinations thereof, wherein the amino acid positions are relative to the reference sequence of SEQ ID NO: 3796.

43. An engineered DNA polymerase or a functional fragment thereof, comprising a polypeptide sequence having at least 75% or more sequence identity to a reference sequence comprising residues 12 to 841 of SEQ ID NO: 4182, or to a reference sequence of SEQ ID NO: 4182, wherein the polypeptide sequence comprises one or more substitutions relative to the reference sequence comprising residues 12 to 841 of SEQ ID NO: 4182, or to the reference sequence of SEQ ID NO: 4182.

44. The engineered DNA polymerase of claim 43, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 4182 or to the reference sequence of SEQ ID NO: 4182.

45. The engineered DNA polymerase of claim 43, comprising one or more substitutions or substitution sets selected from 18/149/300/423/424/743/769, 18/256/423/424/455/673, 18/372/424/533/546/600/673/769, 18/424, 149/256/300/302/424/484/769, 149/256/300/372/424/546, 149/424, 256/300/363/423/424/546/673/690, 424, 424/533, and 424/723, or combinations thereof, wherein the amino acid positions are relative to the reference sequence of SEQ ID NO: 4182.

46. An engineered DNA polymerase or a functional fragment thereof, comprising a polypeptide sequence having at least 75% or more sequence identity to a reference sequence comprising residues 12 to 841 of SEQ ID NO: 5094, or to a reference sequence of SEQ ID NO: 5094, wherein the polypeptide sequence comprises one or more substitutions relative to the reference sequence comprising residues 12 to 841 of SEQ ID NO: 5094, or to the reference sequence of SEQ ID NO: 5094.

47. The engineered DNA polymerase of claim 46, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5094 or to the reference sequence of SEQ ID NO: 5094.

48. The engineered DNA polymerase of claim 46, comprising one or more substitutions or substitution sets selected from 9/689, 9/691, 21/695/700, 45, 45/268, 45/268/302/423, 45/268/302/467/621/748/749, 45/268/302/469, 45/268/302/469/547/557/721/749, 45/268/302/475/489, 45/268/302/547/557/621/748/749, 45/268/423/467/748, 45/268/423/469/721, 45/268/423/475/547/721/749, 45/268/469, 45/268/475, 45/268/475/489, 45/268/475/621, 45/268/489/621/749, 45/268/621/721, 45/302, 45/302/423/469/475, 45/302/423/469/621, 45/302/423/469/621/721/748/749, 45/302/423/547/621/721, 45/302/469/547/721, 45/302/475/621/748/749, 45/302/621, 45/423, 45/423/467, 45/423/467/469/547/721/749, 45/423/467/469/749, 45/423/467/475/547/621/721/749, 45/423/467/621, 45/423/469/475, 45/423/469/475/621/721/749, 45/423/469/489/621, 45/423/475/547/721, 45/423/475/557/621/749, 45/423/489, 45/423/489/557, 45/467/469/557, 45/467/621, 45/467/748, 45/469/721, 45/469/749, 45/475/489/547/749, 45/547/749, 45/557, 45/721, 83/700/744, 95/268/302/423/489/547/557/721/748/749, 116, 228/708, 268, 268/302/423/557/721, 268/302/721, 268/423, 268/423/467/547, 268/423/469, 268/423/469/475, 268/467, 268/467/469/475, 268/467/748, 268/475/547, 268/475/557/721/749, 268/489/547, 296/433, 302, 302/423/467/621/748, 302/423/469/489/721/748/749, 302/423/475/489/547/621, 302/423/547/721, 302/423/557, 302/467/469/547/557, 302/467/489/748/749, 302/467/621, 302/467/721/748, 302/469, 302/469/489/621/721/748, 302/489, 302/489/547/621/699/749, 302/557/721, 302/621, 302/721, 302/749, 306, 306/459, 314/538, 321, 322, 329/359/611/700/744, 332, 335, 339, 341/532, 344, 345, 351, 354/359/408/598, 359, 359/408/412, 359/408/412/611/614/744, 359/408/412/611/708/744, 359/408/412/611/744/789, 359/408/412/700/744, 359/408/412/744, 359/408/433/614/708, 359/408/598/700, 359/408/611/614/744, 359/408/695/700/744, 359/408/695/744, 359/412, 359/412/433/436/598/611/614, 359/412/433/611/614/708, 359/412/433/611/614/744/789, 359/412/436/598/611/695/744, 359/412/469/611/695/708/744, 359/412/598, 359/412/598/611/695/700/708/744, 359/412/598/611/744, 359/412/695/700/744, 359/412/695/708/744, 359/433/611/614/700, 359/433/611/695/700/789, 359/436/611/708/744, 359/469/611, 359/469/611/614, 359/469/611/700, 359/598/611/614/744, 359/598/614/695/708/744, 359/598/700/744, 359/598/744/789, 359/611/614/695/700/744, 359/611/614/744, 359/611/700/744, 359/614, 359/695/700/789, 359/700, 359/744, 359/789, 364, 373, 378, 385, 386, 387/630, 393, 397, 399, 404, 408, 408/412, 408/412/433/436/611/614/744, 408/412/433/598/611/700/708/744, 408/412/436/598/708, 408/412/436/611/614, 408/412/469/475/611/695/708/744, 408/412/598/611/614/708, 408/412/598/700/744, 408/412/611/695/708, 408/412/708/744, 408/433/700/708/744, 408/436/598/611/708/744, 408/436/695/708/744, 408/598/611, 408/598/611/614/700, 408/598/695/700, 408/611/614/695/708, 408/611/614/708/744, 408/611/614/744, 408/611/744, 408/700, 408/744, 409, 412, 412/433/598/611/744, 412/433/611/744, 412/436/611/614/744/789, 412/436/744, 412/469/611/695/744, 412/469/611/700/744, 412/598/611/744, 412/611/614, 412/611/614/708/744/789, 412/611/789, 412/614/708, 412/689, 412/695/789, 412/708/744, 413, 417, 423, 423/467/469, 423/467/489/621/721, 423/467/547/557/621/699/721, 423/467/621/721/749, 423/467/721, 423/469, 423/469/475/489/721/748/749, 423/469/489/557/621/721/749, 423/469/547/557/621/749, 423/469/547/557/749, 423/469/621, 423/469/721, 423/489, 423/489/621, 423/598/700/708/744, 423/721, 423/748, 424, 425, 426, 433, 433/436/598/700/744, 433/436/598/744, 433/436/611/614, 433/469/611, 433/611/695/700/744, 433/744/789, 436, 436/611/614, 436/611/614/695, 436/700/744, 436/708/744, 439, 443, 446, 448, 449, 467/469, 467/489/547/621/748, 467/547/721, 467/621, 467/721, 467/721/748, 467/748, 467/749, 469, 469/471, 469/473, 469/475/621/721/748, 469/475/749, 469/614/695, 469/614/695/744, 469/621, 469/621/748, 469/744, 469/748, 475, 483, 485/791, 487, 489, 506, 507, 509, 511, 514, 517, 520, 532, 533/542, 538, 539, 539/571, 542, 546/699, 547, 547/621/721/748/749, 549, 554, 557, 557/621, 580, 584, 587, 596, 598, 598/611, 598/611/614, 598/611/708, 598/614/700/744, 598/708, 602, 602/724, 611, 611/614, 611/614/708/744/789, 611/744, 614, 614/700/708/744/789, 614/744, 614/789, 617, 621, 621/639/748/749, 621/745, 621/748, 621/749, 630, 652, 689, 691, 695, 695/708/744, 695/744, 699, 700, 700/744, 700/767, 708, 714, 721, 721/749, 723, 725, 741, 744, 745, 748, 748/749, 767, 777, 780, 785, 785/809, 787, 789, 791, 795, 798, 799, 811, 817, 821, 836, 838, and 839, or combinations thereof, wherein the amino acid positions are relative to the reference sequence of SEQ ID NO: 5094.

49. The engineered DNA polymerase of claim 46, comprising one or more substitutions or substitution sets selected from 45/268/302/469, 45/268/475, 45/268/475/489, 45/268/621/721, 45/423/467/469/749, 45/721, 268, 268/475/547, 302, 302/489/547/621/699/749, 302/721, 302/749, 306, 359, 359/412/433/611/614/744/789, 359/436/611/708/744, 385, 408, 412, 412/598/611/744, 417, 423/467/721, 426, 433, 436, 436/708/744, 467/469, 469, 469/475/621/721/748, 469/475/749, 469/621/748, 469/748, 489, 511, 517, 547, 549, 557, 598, 598/611/708, 611, 614, 621/639/748/749, 630, 695, 699, 700, 708, 714, 721, 725, 741, 744, 748, 787, 789, 791, 795, and 817, or combinations thereof, wherein the amino acid positions are relative to the reference sequence of SEQ ID NO: 5094.

50. An engineered DNA polymerase or a functional fragment thereof, comprising a polypeptide sequence having at least 75% or more sequence identity to a reference sequence comprising residues 12 to 841 of SEQ ID NO: 5136, or to a reference sequence of SEQ ID NO: 5136, wherein the polypeptide sequence comprises one or more substitutions relative to the reference sequence comprising residues 12 to 841 of SEQ ID NO: 5136, or to the reference sequence of SEQ ID NO: 5136.

51. The engineered DNA polymerase of claim 50, comprising a polypeptide sequence having at least 80%, 85%, 86%, 87%, 88%, 89%, 90%, 91%, 92%, 93%, 94%, 95%, 96%, 97%, 98%, 99%, or more sequence identity to the reference sequence comprising residues 12 to 841 of SEQ ID NO: 5136 or to the reference sequence of SEQ ID NO: 5136.

52. The engineered DNA polymerase of claim 50, comprising one or more substitutions or substitution sets selected from 359/408/614, 359/412/436/598/611/614/695/700/708/744, 359/412/598/611/614/700/708/744, 359/412/611/708/744, 359/433/744, 359/436/598, 359/469/700/744, 359/598/611, 359/598/611/695/708, 359/598/695, 359/611/700/708, 359/744, 359/789, 408, 408/412/436/614/700/744, 408/412/469, 408/412/611, 408/412/744, 408/433/436/611/614/744, 408/433/436/695/708/744, 408/611, 412, 412/433, 412/611/614/708/744/789, 412/700/744, 433/436/598/611/614/708/744, 433/436/611/614/744, 433/436/611/744, 433/436/744, 433/614/700/744, 433/708, 436/598, 436/598/614/695/744, 436/611, 436/611/614, 436/611/614/695/700/744, 436/611/708, 436/744, 598/611/614/708/744, 598/614/695/700/708/744, 611, 611/614, 611/614/695/700/708/744, 611/708/744, 614/744, 614/789, 695, 695/700/744, 700, 700/744, and 744/789, or combinations thereof, wherein the amino acid positions are relative to the reference sequence of SEQ ID NO: 5136.

53. The engineered DNA polymerase of claim 50, comprising one or more substitutions or substitution sets selected from 359T/408W/614R, 359T/412G/436R/598F/611K/614R/695L/700G/708G/744S, 359T/412G/598F/611P/614R/700G/708G/744S, 359T/412G/611K/708G/744G, 359T/433A/744G, 359T/436R/598F, 359T/469L/700G/744G, 359T/598F/611K, 359T/598F/611K/695L/708G, 359T/598F/695L, 359T/611K/700G/708G, 359T/744S, 359T/789D, 408W, 408W/412G/436R/614R/700G/744S, 408W/412G/469L, 408W/412G/611P, 408W/412G/744G, 408W/433A/436R/611P/614R/744G, 408W/433A/436R/695L/708G/744S, 408W/611P, 412G, 412G/433A, 412G/611K/614R/708G/744S/789D, 412G/700G/744S, 433A/436R/598F/611P/614R/708G/744S, 433A/436R/611P/614R/744S, 433A/436R/611P/744G, 433A/436R/744G, 433A/614R/700G/744S, 433A/708G, 436R/598F, 436R/598F/614R/695L/744G, 436R/611P, 436R/611P/614R, 436R/611P/614R/695L/700G/744S, 436R/611P/708G, 436R/744S, 598F/611P/614R/708G/744G, 598F/614R/695L/700G/708G/744S, 611P, 611P/614R, 611P/614R/695L/700G/708G/744S, 611P/708G/744S, 614R/744G, 614R/789D, 695L, 695L/700G/744G, 700G, 700G/744S, and 744S/789D, or combinations thereof, wherein the amino acid positions are relative to the reference sequence of SEQ ID NO: 5136.

54. The engineered DNA polymerase of claim 50, comprising one or more substitutions or substitution sets selected from 359/598/695, 408/412/436/614/700/744, 408/611, 412, 433/614/700/744, 436/598, 436/611, 436/611/614/695/700/744, and 700, or combinations thereof, wherein the amino acid positions are relative to the reference sequence of SEQ ID NO: 5136.

55. The engineered DNA polymerase of claim 50, comprising one or more substitutions or substitution sets selected from 359T/598F/695L, 408W/412G/436R/614R/700G/744S, 408W/611P, 412G, 433A/614R/700G/744S, 436R/598F, 436R/611P, 436R/611P/614R/695L/700G/744S, and 700G, or combinations thereof, wherein the amino acid positions are relative to the reference sequence of SEQ ID NO: 5136.

56. The engineered DNA polymerase of claim 1, comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to a reference sequence comprising residues 12 to 841 of SEQ ID NO: 178, 994, 1858, 2176, 2310, 2994, 3796, 4182, 5094, and/or 5136, or to a reference sequence of SEQ ID NO: 178, 994, 1858, 2176, 2310, 2994, 3796, 4182, 5094, and/or 5136.

57. The engineered DNA polymerase of claim 1, comprising a polypeptide sequence having at least 75%, 80%, 85%, 86%, 87%, 88%, 89%, 90%, 91%, 92%, 93%, 94%, 95%, 96%, 97%, 98%, 99%, or more sequence identity to a reference sequence comprising residues 12 to 841 of SEQ ID NO: 2, 178, 994, 1858, 2176, 2310, 2994, 3796, 4182, 5094, and/or 5136, or to a reference sequence of SEQ ID NO: 2, 178, 994, 1858, 2176, 2310, 2994, 3796, 4182, 5094, and/or 5136, with the proviso that the polypeptide sequence does not include the sequence comprising residues 12 to 841 of SEQ ID NO:2.

58. The engineered DNA polymerase of claim 57, wherein the polypeptide sequence comprises one or more amino acid substitutions relative to the reference sequence comprising residues 12 to 841 of SEQ ID NO: 178, or to the reference sequence of SEQ ID NO: 178, with the proviso that the polypeptide sequence does not include the sequence comprising residues 12 to 841 of SEQ ID NO: 2.

59. The engineered DNA polymerase of claim 57, wherein the polypeptide sequence comprises one or more amino acid substitutions relative to the reference sequence comprising residues 12 to 841 of SEQ ID NO: 994, or to the reference sequence of SEQ ID NO: 994, with the proviso that the polypeptide sequence does not include the sequence comprising residues 12 to 841 of SEQ ID NO: 2.

60. The engineered DNA polymerase of claim 57, wherein the polypeptide sequence comprises one or more amino acid substitutions relative to the reference sequence comprising residues 12 to 841 of SEQ ID NO: 1858, or to the reference sequence of SEQ ID NO: 1858, with the proviso that the polypeptide sequence does not include the sequence comprising residues 12 to 841 of SEQ ID NO: 2.

61. The engineered DNA polymerase of claim 57, wherein the polypeptide sequence comprises one or more amino acid substitutions relative to the reference sequence comprising residues 12 to 841 of SEQ ID NO: 2176, or to the reference sequence of SEQ ID NO: 2176, with the proviso that the polypeptide sequence does not include the sequence comprising residues 12 to 841 of SEQ ID NO: 2.

62. The engineered DNA polymerase of claim 57, wherein the polypeptide sequence comprises one or more amino acid substitutions relative to the reference sequence comprising residues 12 to 841 of SEQ ID NO: 2310, or to the reference sequence of SEQ ID NO: 2310, with the proviso that the polypeptide sequence does not include the sequence comprising residues 12 to 841 of SEQ ID NO: 2.

63. The engineered DNA polymerase of claim 57, wherein the polypeptide sequence comprises one or more amino acid substitutions relative to the reference sequence comprising residues 12 to 841 of SEQ ID NO: 2994, or to the reference sequence of SEQ ID NO: 2994, with the proviso that the polypeptide sequence does not include the sequence comprising residues 12 to 841 of SEQ ID NO: 2.

64. The engineered DNA polymerase of claim 57, wherein the polypeptide sequence comprises one or more amino acid substitutions relative to the reference sequence comprising residues 12 to 841 of SEQ ID NO: 3796, or to the reference sequence of SEQ ID NO: 3796, with the proviso that the polypeptide sequence does not include the sequence comprising residues 12 to 841 of SEQ ID NO: 2.

65. The engineered DNA polymerase of claim 57, wherein the polypeptide sequence comprises one or more amino acid substitutions relative to the reference sequence comprising residues 12 to 841 of SEQ ID NO: 4182, or to the reference sequence of SEQ ID NO: 4182, with the proviso that the polypeptide sequence does not include the sequence comprising residues 12 to 841 of SEQ ID NO: 2.

66. The engineered DNA polymerase of claim 57, wherein the polypeptide sequence comprises one or more amino acid substitutions relative to the reference sequence comprising residues 12 to 841 of SEQ ID NO: 5094, or to the reference sequence of SEQ ID NO: 5094, with the proviso that the polypeptide sequence does not include the sequence comprising residues 12 to 841 of SEQ ID NO: 2.

67. The engineered DNA polymerase of claim 57, wherein the polypeptide sequence comprises one or more amino acid substitutions relative to the reference sequence comprising residues 12 to 841 of SEQ ID NO: 5136, or to the reference sequence of SEQ ID NO: 5136, with the proviso that the polypeptide sequence does not include the sequence comprising residues 12 to 841 of SEQ ID NO: 2.

68. The engineered DNA polymerase of any of claims 1-67, wherein the DNA polymerase comprises a polypeptide sequence comprising a substitution or substitution set provided in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1, wherein the substitution or substitution set is relative to the reference sequence of SEQ ID NO: 2, 178, 994, 1858, 2176, 2310, 2994, 3796, 4182, 5094, and/or 5136.

69. The engineered DNA polymerase of any of claims 1-68, wherein the engineered DNA polymerase comprises a polypeptide sequence having at least 75%, 80%, 85%, 86%, 87%, 88%, 89%, 90%, 91%, 92%, 93%, 94%, 95%, 96%, 97%, 98%, 99% or more sequence identity to a sequence comprising residues 12 to 841 of an engineered DNA polymerase variant set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1.

70. The engineered DNA polymerase of any of claims 1-69, wherein the DNA polymerase comprises a polypeptide sequence having at least 75%, 80%, 85%, 86%, 87%, 88%, 89%, 90%, 91%, 92%, 93%, 94%, 95%, 96%, 97%, 98%, 99% or more sequence identity to a sequence of an engineered DNA polymerase variant set forth in Table 5.1, 6.1, 7.1, 8.1, 9.1, 10.1, 11.1, 12.1, 13.1, 14.1, 15.1, 16.1, 17.1, 18.1, 19.1, 20.1, 21.1, 22.1, 23.1, 24.1, 25.1, 26.1, 27.1, 28.1, 29.1, 30.1, 31.1, 32.1, 33.1, 34.1, and/or 35.1.

71. The engineered DNA polymerase of any of claims 1-70, wherein the DNA polymerase comprises a polypeptide sequence comprising residues 12 to 841 of a sequence selected from the even numbered sequences of SEQ ID NOS: 4-3258 and 3268-6646.

72. The engineered DNA polymerase of any of claims 1-71, wherein the DNA polymerase comprises a polypeptide sequence comprising residues 12 to 841 of a sequence selected from the even numbered sequences of SEQ ID NOS: 4-3258 and 3268-6646, wherein the polypeptide optionally has 1, 2, 3, 4, 5, 6, 7, 8, 9, or up to 10 substitutions in the polypeptide sequence.

73. The engineered DNA polymerase of any of claims 1-72, wherein the polypeptide has 1, 2, 3, 4, up to 5 substitutions in the polypeptide sequence.

74. The engineered DNA polymerase of claim 73, wherein the substitutions comprise conservative substitutions.

75. The engineered DNA polymerase of claim 1, wherein the polypeptide sequence comprises a sequence comprising residues 12 to 841 of SEQ ID NO: 178, 994, 1858, 2176, 2310, 2994, 3796, 4182, 5094, and/or 5136.

76. The engineered DNA polymerase of claim 1, wherein the polypeptide sequence comprises a sequence of SEQ ID NO: 178, 994, 1858, 2176, 2310, 2994, 3796, 4182, 5094, and/or 5136.

77. The engineered DNA polymerase of any of claims 1-76, wherein the engineered DNA polymerase is a fusion protein.

78. The engineered DNA polymerase of any of claims 1-77, wherein the engineered DNA polymerase has DNA polymerase activity.

79. The engineered DNA polymerase of any of claims 1-78, having at least one improved property as compared to the DNA polymerase of SEQ ID NO: 2.

80. The engineered DNA polymerase of claim 79, having at least one improved property, as compared to the DNA polymerase of SEQ ID NO: 2, wherein the improved property is selected from increased activity, increased stability, increased thermostability, increased processivity, increased fidelity, increased thermotolerance, increase resistance to inhibitors, and increased product yield in a PCR reaction.

81. The engineered DNA polymerase of any of claims 1-80, wherein the DNA polymerase is purified.

82. A polynucleotide encoding an engineered DNA polymerase of any of claims 1-81.

83. A polynucleotide comprising at least 75%, 80%, 85%, 90%, 91%, 92%, 93%, 94%, 95%, 96%, 97%, 98%, 99%, or more sequence identity to a reference polynucleotide sequence of nucleotide residues 34 to 2529 of SEQ ID NO: 1, or to a reference polynucleotide sequence of SEQ ID NO: 1 encoding an engineered DNA polymerase or a functional fragment thereof, wherein the encoded DNA polymerase polypeptide comprises one or more substitutions at one or more amino acid positions relative to the reference sequence comprising residues 12 to 841 of SEQ ID NO: 2, or to a reference sequence of SEQ ID NO: 2.

84. The polynucleotide of claim 82 or 83, wherein the polynucleotide sequence encodes an engineered DNA polymerase comprising a polypeptide sequence having at least 75%, 80%, 85%, 90%, 91%, 92%, 93%, 94%, 95%, 96%, 97%, 98%, 99%, or more sequence identity to a reference sequence comprising residues 12 to 841 of SEQ ID NO: 178, 994, 1858, 2176, 2310, 2994, 3796, 4182, 5094, and/or 5136, or to a reference sequence of SEQ ID NO: 178, 994, 1858, 2176, 2310, 2994, 3796, 4182, 5094, and/or 5136.

85. The polynucleotide of claim 84, wherein the polynucleotide sequence comprises nucleotide residues 12 to 841 of SEQ ID NO: 177, 993, 1857, or 2175, 2309, 2993, 3795, 4181, 5093, or 5135, or comprises the polynucleotide sequence of SEQ ID NO: 177, 993, 1857, or 2175, 2309, 2993, 3795, 4181, 5093, or 5135.

86. The polynucleotide of any of claims 82-85, wherein the polynucleotide sequence comprises nucleotide residues 34 to 2529 of the odd-numbered sequences of SEQ ID NOS: 3-3257 or 3267-6645.

87. The polynucleotide of any of claims 82-86, wherein the polynucleotide sequence comprises the odd-numbered sequences of SEQ ID NOS: 3-3257 or 3267-6645.

88. The polynucleotide sequence of any of claims 82-87, wherein the polynucleotide sequence is operably linked to a control sequence.

89. The polynucleotide sequence of any of claims 82-88, wherein the polynucleotide sequence is codon-optimized.

90. An expression vector comprising at least one polynucleotide sequence of any of claims 82-89.

91. A host cell transformed with a polynucleotide of any of claims 82-89.

92. A host cell transformed with the expression vector of claim 90.

93. A method of producing an engineered DNA polymerase polypeptide in a host cell comprising culturing a host cell of claim 91, under suitable culture conditions, such that at least one engineered DNA polymerase is produced.

94. A method of producing an engineered DNA polymerase polypeptide in a host cell comprising culturing a host cell of claim 92, under suitable culture conditions, such that at least one engineered DNA polymerase is produced.

95. The method of claim 93 or 94, further comprising recovering at least one engineered DNA polymerase from the culture and/or host cells.

96. The method of claim 94 or 95, further comprising a step of purifying the at least one engineered DNA polymerase.

97. A composition comprising at least one engineered DNA polymerase of any of claims 1-81.

98. The composition of claim 97, comprising at least one buffer.

99. The composition of claim 97 or 98, further comprising one or more nucleotide substrates.

100. The composition of any of claims 97-99, further comprising one or more oligonucleotide primer substrates.

101. A method of preparing a complementary DNA of a target DNA, comprising contacting a target DNA with an engineered DNA polymerase of any one of claims 1-81, in presence of substrates under conditions suitable for production of a complementary DNA to all or a portion of the target DNA.

102. A method for detecting presence of a target DNA, comprising contacting a sample suspected of containing a target DNA with an engineered DNA polymerase of any one of claims 1-81, in presence of substrates under conditions suitable for DNA polymerase mediated production of a DNA complementary to all or a portion of the target DNA, and detecting presence of the complementary DNA.

103. The method of claim 101 or 102, wherein the complementary DNA is detected by amplifying the complementary DNA.

104. The method of claim 103, wherein the amplifying is by polymerase chain reaction (PCR) or isothermal amplification.

105. The method of claim 104, wherein the isothermal amplification is by loop-mediated isothermal amplification (LAMP).

106. A method of amplifying a target DNA, comprising contacting a target DNA with a DNA polymerase of any one of claims 1-81, in presence of substrates under conditions suitable for amplifying the target DNA.

107. The method of claim 106, wherein the conditions are for a polymerase chain reaction.

108. The method of claim 106, wherein the conditions is for LAMP.

109. A method of sequencing a target DNA, comprising contacting a target DNA with a DNA polymerase of any one of claims 1-81, in presence of substrates appropriate for sequencing under conditions suitable for DNA polymerase mediated extension of a complementary DNA of the target DNA, and determining the sequence of the target DNA.

110. A kit comprising at least one engineered DNA polymerase of any one of claims 1-81.

111. The kit of claim 110, further comprising at least one buffer, nucleotide substrate, and/or an oligonucleotide primer substrate.

112. The kit of claim 110 or 111, further comprising a second DNA polymerase.
